SEQUENCE LISTING (560 sequences)

SEQ ID NO: 1 - DNA, 178 bp, synthetic construct
  misc_feature 1..178: note = Description of Artificial Sequence: Synthetic polynucleotide
  source 1..178: mol_type = other DNA
  CDS 2..178
gtgctggcatgccgcgccctcccagaggcccaccttcaagcagctggtggaggacctggaccgtgtccttaccgtgacgtccaccgactttaaggagtcggccttgaggaagcagtccttatacctcaagttcgaccccctcctgagggacagtcctggtagaccagtgcccgtggcc

SEQ ID NO: 2 - DNA, 91 bp, synthetic construct
  misc_feature 1..91: note = Description of Artificial Sequence: Synthetic oligonucleotide
  source 1..91: mol_type = other DNA
ctttaaggagtcggccttgaggaagcagtccttatacctcaagttcgaccccctcctgagggacagtcctggtagaccagtgcccgtggcc

SEQ ID NO: 3 - DNA, 91 bp, synthetic construct
  misc_feature 1..91: note = Description of Artificial Sequence: Synthetic oligonucleotide
  source 1..91: mol_type = other DNA
actttaaggagtcggccttgaggaagcagtccttatacctcaagttcgaccccctcctgagggacagtcctggtagaccagtgcccgtggg

SEQ ID NO: 4 - DNA, 91 bp, synthetic construct
  misc_feature 1..91: note = Description of Artificial Sequence: Synthetic oligonucleotide
  source 1..91: mol_type = other DNA
gactttaaggagtcggccttgaggaagcagtccttatacctcaagttcgaccccctcctgagggacagtcctggtagaccagtgcccgtgg

SEQ ID NO: 5 - DNA, 91 bp, synthetic construct
  misc_feature 1..91: note = Description of Artificial Sequence: Synthetic oligonucleotide
  source 1..91: mol_type = other DNA
cgactttaaggagtcggccttgaggaagcagtccttatacctcaagttcgaccccctcctgagggacagtcctggtagaccagtgcccgtt

SEQ ID NO: 6 - DNA, 91 bp, synthetic construct
  misc_feature 1..91: note = Description of Artificial Sequence: Synthetic oligonucleotide
  source 1..91: mol_type = other DNA
ccgactttaaggagtcggccttgaggaagcagtccttatacctcaagttcgaccccctcctgagggacagtcctggtagaccagtgcccgg

SEQ ID NO: 7 - DNA, 91 bp, synthetic construct
  misc_feature 1..91: note = Description of Artificial Sequence: Synthetic oligonucleotide
  source 1..91: mol_type = other DNA
accgactttaaggagtcggccttgaggaagcagtccttatacctcaagttcgaccccctcctgagggacagtcctggtagaccagtgcccc

SEQ ID NO: 8 - DNA, 91 bp, synthetic construct
  misc_feature 1..91: note = Description of Artificial Sequence: Synthetic oligonucleotide
  source 1..91: mol_type = other DNA
caccgactttaaggagtcggccttgaggaagcagtccttatacctcaagttcgaccccctcctgagggacagtcctggtagaccagtgccc

SEQ ID NO: 9 - DNA, 91 bp, synthetic construct
  misc_feature 1..91: note = Description of Artificial Sequence: Synthetic oligonucleotide
  source 1..91: mol_type = other DNA
ccaccgactttaaggagtcggccttgaggaagcagtccttatacctcaagttcgaccccctcctgagggacagtcctggtagaccagtgcc

SEQ ID NO: 10 - DNA, 91 bp, synthetic construct
  misc_feature 1..91: note = Description of Artificial Sequence: Synthetic oligonucleotide
  source 1..91: mol_type = other DNA
tccaccgactttaaggagtcggccttgaggaagcagtccttatacctcaagttcgaccccctcctgagggacagtcctggtagaccagtgg

SEQ ID NO: 11 - DNA, 91 bp, synthetic construct
  misc_feature 1..91: note = Description of Artificial Sequence: Synthetic oligonucleotide
  source 1..91: mol_type = other DNA
gtccaccgactttaaggagtcggccttgaggaagcagtccttatacctcaagttcgaccccctcctgagggacagtcctggtagaccagtt

SEQ ID NO: 12 - DNA, 91 bp, synthetic construct
  misc_feature 1..91: note = Description of Artificial Sequence: Synthetic oligonucleotide
  source 1..91: mol_type = other DNA
cgtccaccgactttaaggagtcggccttgaggaagcagtccttatgcctcaagttcggccccctcctgagggacagtcctggtagaccagg

SEQ ID NO: 13 - DNA, 90 bp, synthetic construct
  misc_feature 1..90: note = Description of Artificial Sequence: Synthetic oligonucleotide
  source 1..90: mol_type = other DNA
gacgtccaccgactttaaggagtcggccttgaggaagcagtccttatacctcaagttcgaccccctcctgagggacagtcctggtagacg

SEQ ID NO: 14 - DNA, 91 bp, synthetic construct
  misc_feature 1..91: note = Description of Artificial Sequence: Synthetic oligonucleotide
  source 1..91: mol_type = other DNA
tgacgtccaccgactttaaggagtcggccttgaggaagcagtccttatacctcaagttcgaccccctcctgagggacagtcctggtagacc

SEQ ID NO: 15 - DNA, 91 bp, synthetic construct
  misc_feature 1..91: note = Description of Artificial Sequence: Synthetic oligonucleotide
  source 1..91: mol_type = other DNA
gtgacgtccaccgactttaaggagtcggccttgaggaagcagtccttatacctcaagttcgaccccctcctgagggacagtcctggtagaa

SEQ ID NO: 16 - DNA, 91 bp, synthetic construct
  misc_feature 1..91: note = Description of Artificial Sequence: Synthetic oligonucleotide
  source 1..91: mol_type = other DNA
cgtgacgtccaccgactttaaggagtcggccttgaggaagcagtccttatacctcaagttcgaccccctcctgagggacagtcctggtagg

SEQ ID NO: 17 - DNA, 91 bp, synthetic construct
  misc_feature 1..91: note = Description of Artificial Sequence: Synthetic oligonucleotide
  source 1..91: mol_type = other DNA
ccgtgacgtccaccgactttaaggagtcggccttgaggaagcagtccttatacctcaagttcgaccccctcctgagggacagtcctggtaa

SEQ ID NO: 18 - DNA, 91 bp, synthetic construct
  misc_feature 1..91: note = Description of Artificial Sequence: Synthetic oligonucleotide
  source 1..91: mol_type = other DNA
cttaccgtgacgtccaccgactttaaggagtcggccttgaggaagcagtccttatacctcaagttcgaccccctcctgagggacagtcctt

SEQ ID NO: 19 - DNA, 91 bp, synthetic construct
  misc_feature 1..91: note = Description of Artificial Sequence: Synthetic oligonucleotide
  source 1..91: mol_type = other DNA
gtccttaccgtgacgtccaccgactttaaggagtcggccttgaggaagcagtccttatacctcaagttcgaccccctcctgagggacagtt

SEQ ID NO: 20 - DNA, 91 bp, synthetic construct
  misc_feature 1..91: note = Description of Artificial Sequence: Synthetic oligonucleotide
  source 1..91: mol_type = other DNA
tgtccttaccgtgacgtccaccgactttaaggagtcggccttgaggaagcagtccttatacctcaagttcgaccccctcctgagggacagg

SEQ ID NO: 21 - DNA, 91 bp, synthetic construct
  misc_feature 1..91: note = Description of Artificial Sequence: Synthetic oligonucleotide
  source 1..91: mol_type = other DNA
gtgtccttaccgtgacgtccaccgactttaaggagtcggccttgaggaagcagtccttatacctcaagttcgaccccctcctgagggacaa

SEQ ID NO: 22 - DNA, 91 bp, synthetic construct
  misc_feature 1..91: note = Description of Artificial Sequence: Synthetic oligonucleotide
  source 1..91: mol_type = other DNA
cgtgtccttaccgtgacgtccaccgactttaaggagtcggccttgaggaagcagtccttatacctcaagttcgaccccctcctgagggacc

SEQ ID NO: 23 - DNA, 91 bp, synthetic construct
  misc_feature 1..91: note = Description of Artificial Sequence: Synthetic oligonucleotide
  source 1..91: mol_type = other DNA
accgtgtccttaccgtgacgtccaccgactttaaggagtcggccttgaggaagcagtccttatacctcaagttcgaccccctcctgagggg

SEQ ID NO: 24 - DNA, 91 bp, synthetic construct
  misc_feature 1..91: note = Description of Artificial Sequence: Synthetic oligonucleotide
  source 1..91: mol_type = other DNA
gaccgtgtccttaccgtgacgtccaccgactttaaggagtcggccttgaggaagcagtccttatacctcaagttcgaccccctcctgaggg

SEQ ID NO: 25 - DNA, 91 bp, synthetic construct
  misc_feature 1..91: note = Description of Artificial Sequence: Synthetic oligonucleotide
  source 1..91: mol_type = other DNA
ggaccgtgtccttaccgtgacgtccaccgactttaaggagtcggccttgaggaagcagtccttatacctcaagttcgaccccctcctgagg

SEQ ID NO: 26 - DNA, 91 bp, synthetic construct
  misc_feature 1..91: note = Description of Artificial Sequence: Synthetic oligonucleotide
  source 1..91: mol_type = other DNA
ctggaccgtgtccttaccgtgacgtccaccgactttaaggagtcggccttgaggaagcagtccttatacctcaagttcgaccccctcctgg

SEQ ID NO: 27 - DNA, 91 bp, synthetic construct
  misc_feature 1..91: note = Description of Artificial Sequence: Synthetic oligonucleotide
  source 1..91: mol_type = other DNA
cctggaccgtgtccttaccgtgacgtccaccgactttaaggagtcggccttgaggaagcagtccttatacctcaagttcgaccccctcctt

SEQ ID NO: 28 - DNA, 91 bp, synthetic construct
  misc_feature 1..91: note = Description of Artificial Sequence: Synthetic oligonucleotide
  source 1..91: mol_type = other DNA
gacctggaccgtgtccttaccgtgacgtccaccgactttaaggagtcggccttgaggaagcagtccttatacctcaagttcgaccccctcc

SEQ ID NO: 29 - DNA, 91 bp, synthetic construct
  misc_feature 1..91: note = Description of Artificial Sequence: Synthetic oligonucleotide
  source 1..91: mol_type = other DNA
ggacctggaccgtgtccttaccgtgacgtccaccgactttaaggagtcggccttgaggaagcagtccttatacctcaagttcgaccccctt

SEQ ID NO: 30 - DNA, 91 bp, synthetic construct
  misc_feature 1..91: note = Description of Artificial Sequence: Synthetic oligonucleotide
  source 1..91: mol_type = other DNA
aggacctggaccgtgtccttaccgtgacgtccaccgactttaaggagtcggccttgaggaagcagtccttatacctcaagttcgacccccc

SEQ ID NO: 31 - DNA, 90 bp, synthetic construct
  misc_feature 1..90: note = Description of Artificial Sequence: Synthetic oligonucleotide
  source 1..90: mol_type = other DNA
gaggacctggaccgtgtccttaccgtgacgtccaccgactttaaggagtcggccttgaggaagcagtccttatacctcaagttcgacccc

SEQ ID NO: 32 - DNA, 90 bp, synthetic construct
  misc_feature 1..90: note = Description of Artificial Sequence: Synthetic oligonucleotide
  source 1..90: mol_type = other DNA
ggaggacctggaccgtgtccttaccgtgacgtccaccgactttaaggagtcggccttgaggaagcagtccttatacctcaagttcgaccc

SEQ ID NO: 33 - DNA, 90 bp, synthetic construct
  misc_feature 1..90: note = Description of Artificial Sequence: Synthetic oligonucleotide
  source 1..90: mol_type = other DNA
tggaggacctggaccgtgtccttaccgtgacgtccaccgactttaaggagtcggccttgaggaagcagtccttatacctcaagttcgacc

SEQ ID NO: 34 - DNA, 90 bp, synthetic construct
  misc_feature 1..90: note = Description of Artificial Sequence: Synthetic oligonucleotide
  source 1..90: mol_type = other DNA
gtggaggacctggaccgtgtccttaccgtgacgtccaccgactttaaggagtcggccttgaggaagcagtccttatacctcaagttcgac

SEQ ID NO: 35 - DNA, 90 bp, synthetic construct
  misc_feature 1..90: note = Description of Artificial Sequence: Synthetic oligonucleotide
  source 1..90: mol_type = other DNA
ggtggaggacctggaccgtgtccttaccgtgacgtccaccgactttaaggagtcggccttgaggaagcagtccttatacctcaagttcga

SEQ ID NO: 36 - DNA, 90 bp, synthetic construct
  misc_feature 1..90: note = Description of Artificial Sequence: Synthetic oligonucleotide
  source 1..90: mol_type = other DNA
tggtggaggacctggaccgtgtccttaccgtgacgtccaccgactttaaggagtcggccttgaggaagcagtccttatacctcaagttcg

SEQ ID NO: 37 - DNA, 90 bp, synthetic construct
  misc_feature 1..90: note = Description of Artificial Sequence: Synthetic oligonucleotide
  source 1..90: mol_type = other DNA
ctggtggaggacctggaccgtgtccttaccgtgacgtccaccgactttaaggagtcggccttgaggaagcagtccttatacctctaatca

SEQ ID NO: 38 - DNA, 90 bp, synthetic construct
  misc_feature 1..90: note = Description of Artificial Sequence: Synthetic oligonucleotide
  source 1..90: mol_type = other DNA
gctggtggaggacctggaccgtgtccttaccgtgacgtccaccggctttaaggagtcggcctcgaggaagcagcccttttacctcaagtt

SEQ ID NO: 39 - DNA, 90 bp, synthetic construct
  misc_feature 1..90: note = Description of Artificial Sequence: Synthetic oligonucleotide
  source 1..90: mol_type = other DNA
agctggtggaggacctggaccgtgtccttaccgtgacgtccaccgactttaaggagtcggccttgaggaagcagtccttatacctcaagt

SEQ ID NO: 40 - DNA, 90 bp, synthetic construct
  misc_feature 1..90: note = Description of Artificial Sequence: Synthetic oligonucleotide
  source 1..90: mol_type = other DNA
cagctggtggaggacctggaccgtgtccttaccgtgacgtccaccgactttaaggagtcggccttgaggaagcagtccttatacctcaag

SEQ ID NO: 41 - DNA, 90 bp, synthetic construct
  misc_feature 1..90: note = Description of Artificial Sequence: Synthetic oligonucleotide
  source 1..90: mol_type = other DNA
gcagctggtggaggacctggaccgtgtccttaccgtgacgtccaccgactttaaggagtcggccttgaggaagcagtccttatacctcaa

SEQ ID NO: 42 - DNA, 90 bp, synthetic construct
  misc_feature 1..90: note = Description of Artificial Sequence: Synthetic oligonucleotide
  source 1..90: mol_type = other DNA
agcagctggtggaggacctggaccgtgtccttaccgtgacgtccaccgactttaaggagtcggccttgaggaagcagtccttatacctca

SEQ ID NO: 43 - DNA, 90 bp, synthetic construct
  misc_feature 1..90: note = Description of Artificial Sequence: Synthetic oligonucleotide
  source 1..90: mol_type = other DNA
aagcagctggtggaggacctggaccgtgtccttaccgtgacgtccaccgactttaaggagtcggccttgaggaagcagtccctatacccc

SEQ ID NO: 44 - DNA, 90 bp, synthetic construct
  misc_feature 1..90: note = Description of Artificial Sequence: Synthetic oligonucleotide
  source 1..90: mol_type = other DNA
caagcagctggtggaggacctggaccgtgtccttaccgtgacgtccaccgactttaaggagtcggccttgaggaagcagtccttatacct

SEQ ID NO: 45 - DNA, 90 bp, synthetic construct
  misc_feature 1..90: note = Description of Artificial Sequence: Synthetic oligonucleotide
  source 1..90: mol_type = other DNA
tcaagcagctggtggaggacctggaccgtgtccttaccgtgacgtccaccgactttaaggagtcggccttgaggaagcagtccttatacc

SEQ ID NO: 46 - DNA, 90 bp, synthetic construct
  misc_feature 1..90: note = Description of Artificial Sequence: Synthetic oligonucleotide
  source 1..90: mol_type = other DNA
ttcaagcagctggtggaggacctggaccgtgtccttaccgtgacgtccaccgactttaaggagtcggccttgaggaagcagtccttatac

SEQ ID NO: 47 - DNA, 90 bp, synthetic construct
  misc_feature 1..90: note = Description of Artificial Sequence: Synthetic oligonucleotide
  source 1..90: mol_type = other DNA
cttcaagcagctggtggaggacctggaccgtgtccttaccgtgacgtccaccgactttaaggagtcggccttgaggaagcagtccttata

SEQ ID NO: 48 - DNA, 90 bp, synthetic construct
  misc_feature 1..90: note = Description of Artificial Sequence: Synthetic oligonucleotide
  source 1..90: mol_type = other DNA
ccttcaagcagctggtggaggacctggaccgtgtccttaccgtgacgtccaccgactttaaggagtcggccttgaggaagcagtccttat

SEQ ID NO: 49 - DNA, 90 bp, synthetic construct
  misc_feature 1..90: note = Description of Artificial Sequence: Synthetic oligonucleotide
  source 1..90: mol_type = other DNA
accttcaagcagctggtggaggacctggaccgtgtccttaccgtgacgtccaccgactttaaggagtcggccttgaggaagcagtcctta

SEQ ID NO: 50 - DNA, 90 bp, synthetic construct
  misc_feature 1..90: note = Description of Artificial Sequence: Synthetic oligonucleotide
  source 1..90: mol_type = other DNA
caccttcaagcagctggtggaggacctggaccgtgtccttaccgtgacgtccaccgactttaaggagtcggccttgaggaagcagtcctt

SEQ ID NO: 51 - DNA, 90 bp, synthetic construct
  misc_feature 1..90: note = Description of Artificial Sequence: Synthetic oligonucleotide
  source 1..90: mol_type = other DNA
ccaccttcaagcagctggtggaggacctggaccgtgtccttaccgtgacgtccaccgactttaaggagtcggccttgaggaagcagtcct

SEQ ID NO: 52 - DNA, 90 bp, synthetic construct
  misc_feature 1..90: note = Description of Artificial Sequence: Synthetic oligonucleotide
  source 1..90: mol_type = other DNA
cccaccttcaagcagctggtggaggacctggaccgtgtccttaccgtgacgtccaccgactttaaggagtcggccttgaggaagcagtcc

SEQ ID NO: 53 - DNA, 90 bp, synthetic construct
  misc_feature 1..90: note = Description of Artificial Sequence: Synthetic oligonucleotide
  source 1..90: mol_type = other DNA
ggcccaccttcaagcagctggtggaggacctggaccgtgtccttaccgtgacgtccaccgactttaaggagtcggccttgaggaagcagt

SEQ ID NO: 54 - DNA, 90 bp, synthetic construct
  misc_feature 1..90: note = Description of Artificial Sequence: Synthetic oligonucleotide
  source 1..90: mol_type = other DNA
aggcccaccttcaagcagctggtggaggacctggaccgtgtccttaccgtgacgtccaccgactttaaggagtcggccttgaggaagcag

SEQ ID NO: 55 - DNA, 90 bp, synthetic construct
  misc_feature 1..90: note = Description of Artificial Sequence: Synthetic oligonucleotide
  source 1..90: mol_type = other DNA
gaggcccaccttcaagcagctggtggaggacctggaccgtgtccttaccgtgacgtccaccgactttaaggagtcggccttgaggaagca

SEQ ID NO: 56 - DNA, 90 bp, synthetic construct
  misc_feature 1..90: note = Description of Artificial Sequence: Synthetic oligonucleotide
  source 1..90: mol_type = other DNA
agaggcccaccttcaagcagctggtggaggtcctggaccgtgtccttaccgtgacgtccaccgactttaaggagtcggccttgaggaagc

SEQ ID NO: 57 - DNA, 90 bp, synthetic construct
  misc_feature 1..90: note = Description of Artificial Sequence: Synthetic oligonucleotide
  source 1..90: mol_type = other DNA
cagaggcccaccttcaagcagctggtggaggacctggaccgtgtccttaccgtgacgtccaccgactttaaggagtcggccttgaggaag

SEQ ID NO: 58 - DNA, 90 bp, synthetic construct
  misc_feature 1..90: note = Description of Artificial Sequence: Synthetic oligonucleotide
  source 1..90: mol_type = other DNA
ccagaggcccaccttcaagcagctggtggaggacctggaccgtgtccttaccgtgacgtccaccgactttaaggagtcggccttgaggaa

SEQ ID NO: 59 - DNA, 90 bp, synthetic construct
  misc_feature 1..90: note = Description of Artificial Sequence: Synthetic oligonucleotide
  source 1..90: mol_type = other DNA
cccagaggcccaccttcaagcagctggtggaggacctggaccgtgtccttaccgtgacgtccaccgactttaaggagtcggccttgagga

SEQ ID NO: 60 - DNA, 90 bp, synthetic construct
  misc_feature 1..90: note = Description of Artificial Sequence: Synthetic oligonucleotide
  source 1..90: mol_type = other DNA
tcccagaggcccaccttcaagcagctggtggaggacctggaccgtgtccttaccgtgacgtccaccgactttaaggagtcggccttgagg

SEQ ID NO: 61 - DNA, 90 bp, synthetic construct
  misc_feature 1..90: note = Description of Artificial Sequence: Synthetic oligonucleotide
  source 1..90: mol_type = other DNA
ctcccagaggcccaccttcaagcagctggtggaggacctggaccgtgtccttaccgtgacgtccaccgactttaaggagtcggccttgag

SEQ ID NO: 62 - DNA, 90 bp, synthetic construct
  misc_feature 1..90: note = Description of Artificial Sequence: Synthetic oligonucleotide
  source 1..90: mol_type = other DNA
cctcccagaggcccaccttcaagcagctggtggaggacctggaccgtgtccttaccgtgacgtccaccgactttaaggagtcggccttga

SEQ ID NO: 63 - DNA, 90 bp, synthetic construct
  misc_feature 1..90: note = Description of Artificial Sequence: Synthetic oligonucleotide
  source 1..90: mol_type = other DNA
ccctcccagaggcccaccttcaagcagctggtggaggacctggaccgtgtccttaccgtgacgtccaccgactttaaggagtcggccttg

SEQ ID NO: 64 - DNA, 90 bp, synthetic construct
  misc_feature 1..90: note = Description of Artificial Sequence: Synthetic oligonucleotide
  source 1..90: mol_type = other DNA
gcgccctcccagaggcccaccttcaagcagctggtggaggacctggaccgtgtccttaccgtgacgtccaccgactttaaggagtcggcc

SEQ ID NO: 65 - DNA, 90 bp, synthetic construct
  misc_feature 1..90: note = Description of Artificial Sequence: Synthetic oligonucleotide
  source 1..90: mol_type = other DNA
cgcgccctcccagaggcccaccttcaagcagctggtggaggacctggaccgtgtccttaccgtgacgtccaccgactttaaggagtcggc

SEQ ID NO: 66 - DNA, 90 bp, synthetic construct
  misc_feature 1..90: note = Description of Artificial Sequence: Synthetic oligonucleotide
  source 1..90: mol_type = other DNA
ccgcgccctcccagaggcccaccttcaagcagctggtggaggacctggaccgtgtccttaccgtgacgtccaccgactttaaggagtcgg

SEQ ID NO: 67 - DNA, 90 bp, synthetic construct
  misc_feature 1..90: note = Description of Artificial Sequence: Synthetic oligonucleotide
  source 1..90: mol_type = other DNA
gccgcgccctcccagaggcccaccttcaagcagctggtggaggacctggaccgtgtccttaccgtgacgtccaccgactttaaggagtcg

SEQ ID NO: 68 - DNA, 90 bp, synthetic construct
  misc_feature 1..90: note = Description of Artificial Sequence: Synthetic oligonucleotide
  source 1..90: mol_type = other DNA
tgccgcgccctcccagaggcccaccttcaagcagctggtggaggacctggaccgtgtccttaccgtgacgtccaccgactttaaggagtc

SEQ ID NO: 69 - DNA, 90 bp, synthetic construct
  misc_feature 1..90: note = Description of Artificial Sequence: Synthetic oligonucleotide
  source 1..90: mol_type = other DNA
atgccgcgccctcccagaggcccaccttcaagcagctggtggaggacctggaccgtgtccttaccgtgacgtccaccgactttaaggagt

SEQ ID NO: 70 - DNA, 90 bp, synthetic construct
  misc_feature 1..90: note = Description of Artificial Sequence: Synthetic oligonucleotide
  source 1..90: mol_type = other DNA
catgccgcgccctcccagaggcccaccttcaagcagctggtggaggacctggaccgtgtccttaccgtgacgtccaccgactttaaggag

SEQ ID NO: 71 - DNA, 90 bp, synthetic construct
  misc_feature 1..90: note = Description of Artificial Sequence: Synthetic oligonucleotide
  source 1..90: mol_type = other DNA
gcatgccgcgccctcccagaggcccaccttcaagcagctggtggaggacctggaccgtgtccttaccgtgacgtccaccgactttaagga

SEQ ID NO: 72 - DNA, 90 bp, synthetic construct
  misc_feature 1..90: note = Description of Artificial Sequence: Synthetic oligonucleotide
  source 1..90: mol_type = other DNA
ggcatgccgcgccctcccagaggcccaccttcaagcagctggtggaggacctggaccgtgtccttaccgtgacgtccaccgactttaagg

SEQ ID NO: 73 - DNA, 90 bp, synthetic construct
  misc_feature 1..90: note = Description of Artificial Sequence: Synthetic oligonucleotide
  source 1..90: mol_type = other DNA
tggcatgccgcgccctcccagaggcccaccttcaagcagctggtggaggacctggaccgtgtccttaccgtgacgtccaccgactttaag

SEQ ID NO: 74 - DNA, 90 bp, synthetic construct
  misc_feature 1..90: note = Description of Artificial Sequence: Synthetic oligonucleotide
  source 1..90: mol_type = other DNA
ctggcatgccgcgccctcccagaggcccaccttcaagcagctggtggaggacctggaccgtgtccttaccgtgacgtccaccgactttaa

SEQ ID NO: 75 - DNA, 90 bp, synthetic construct
  misc_feature 1..90: note = Description of Artificial Sequence: Synthetic oligonucleotide
  source 1..90: mol_type = other DNA
gctggcatgccgcgccctcccagaggcccaccttcaagcagctggtggaggacctggaccgtgtccttaccgtgacgtccaccgacttta

SEQ ID NO: 76 - DNA, 90 bp, synthetic construct
  misc_feature 1..90: note = Description of Artificial Sequence: Synthetic oligonucleotide
  source 1..90: mol_type = other DNA
tgctggcatgccgcgccctcccagaggcccaccttcaagcagctggtggaggacctggaccgtgtccttaccgtgacgtccaccgacttt

SEQ ID NO: 77 - DNA, 90 bp, synthetic construct
  misc_feature 1..90: note = Description of Artificial Sequence: Synthetic oligonucleotide
  source 1..90: mol_type = other DNA
gtgctggcatgccgcgccctcccagaggcccaccttcaagcagctggtggaggacctggaccgtgtccttaccgtgacgtccaccgactt

SEQ ID NO: 78 - protein, 59 aa, synthetic construct
  REGION 1..59: note = Description of Artificial Sequence: Synthetic polypeptide
CWHAAPSQRPTFKQLVEDLDRVLTVTSTDFKESALRKQSLYLKFDPLLRDSPGRPVPVA

SEQ ID NO: 79 - protein, 1048 aa, synthetic construct
  REGION 1..1048: note = Description of Artificial Sequence: Synthetic polypeptide
MGAPACALALCVAVAIVAGASSESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAGILSYGVGFFLFILVVAAVTLCRLRSPPKKGLGSPTVHKISRFPLKRQVSLESNASMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREFLRARRPPGLDYSFDTCKPPEEQLTFKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAAPSQRPTFKQLVEDLDRVLTVTSTDFKESALRKQSLYLKFDPLLRDSPGRPVPVATETSSMHGANETPSGRPREAKLVEFDFLGALDIPVPGPPPGVPAPGGPPLSTGPIVDLLQYSQKDLDAVVKATQEENRELRSRCEELHGKNLELGKIMDRFEEVVYQAMEEVQKQKELSKAEIQKVLKEKDQLTTDLNSMEKSFSDLFKRFEKQKEVIEGYRKNEESLKKCVEDYLARITQEGQRYQALKAHAEEKLQLANEEIAQVRSKAQAEALALQASLRKEQMRIQSLEKTVEQKTKENEELTRICDDLISKMEKI

SEQ ID NO: 80 - DNA, 26 bp, synthetic construct
  misc_feature 1..26: note = Description of Artificial Sequence: Synthetic oligonucleotide
  source 1..26: mol_type = other DNA
  CDS 2..25
gacgtccaccgactttaaggagtcgg

SEQ ID NO: 81 - DNA, 27 bp, synthetic construct
  misc_feature 1..27: note = Description of Artificial Sequence: Synthetic oligonucleotide
  source 1..27: mol_type = other DNA
  CDS 2..25
gacgtccaccgacgtaaaggcgacaca

SEQ ID NO: 82 - DNA, 27 bp, synthetic construct
  misc_feature 1..27: note = Description of Artificial Sequence: Synthetic oligonucleotide
  source 1..27: mol_type = other DNA
  CDS 1..27
gccgtccccggccatccctcaggacgt

SEQ ID NO: 83 - DNA, 26 bp, synthetic construct
  misc_feature 1..26: note = Description of Artificial Sequence: Synthetic oligonucleotide
  source 1..26: mol_type = other DNA
  CDS 2..25
gacctccaaccaggggctgctggagt

SEQ ID NO: 84 - DNA, 27 bp, synthetic construct
  misc_feature 1..27: note = Description of Artificial Sequence: Synthetic oligonucleotide
  source 1..27: mol_type = other DNA
  CDS 2..25
gacgtccaccgacgtgccaggcccacc

SEQ ID NO: 85 - protein, 8 aa, synthetic construct
  REGION 1..8: note = Description of Artificial Sequence: Synthetic peptide
TSTDFKES

SEQ ID NO: 86 - protein, 8 aa, synthetic construct
  REGION 1..8: note = Description of Artificial Sequence: Synthetic peptide
TSTDVKAT

SEQ ID NO: 87 - protein, 9 aa, synthetic construct
  REGION 1..9: note = Description of Artificial Sequence: Synthetic peptide
AVPGHPSGR

SEQ ID NO: 88 - protein, 8 aa, synthetic construct
  REGION 1..8: note = Description of Artificial Sequence: Synthetic peptide
TSNQGLLE

SEQ ID NO: 89 - protein, 8 aa, synthetic construct
  REGION 1..8: note = Description of Artificial Sequence: Synthetic peptide
TSTDVPGP

SEQ ID NO: 90 - protein, 806 aa, Homo sapiens
MGAPACALALCVAVAIVAGASSESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAGILSYGVGFFLFILVVAAVTLCRLRSPPKKGLGSPTVHKISRFPLKRQVSLESNASMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREFLRARRPPGLDYSFDTCKPPEEQLTFKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAAPSQRPTFKQLVEDLDRVLTVTSTDEYLDLSAPFEQYSPGGQDTPSSSSSGDDSVFAHDLLPPAPPSSGGSRT

SEQ ID NO: 91 - DNA, 4304 bp, Homo sapiens
  source 1..4304: mol_type = unassigned DNA
gtcgcgggcagctggcgccgcgcggtcctgctctgccggtcgcacggacgcaccggcgggccgccggccggagggacggggcgggagctgggcccgcggacagcgagccggagcgggagccgcgcgtagcgagccgggctccggcgctcgccagtctcccgagcggcgcccgcctcccgccggtgcccgcgccgggccgtggggggcagcatgcccgcgcgcgctgcctgaggacgccgcggcccccgcccccgccatgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacaaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacgagtacctggacctgtcggcgcctttcgagcagtactccccgggtggccaggacacccccagctccagctcctcaggggacgactccgtgtttgcccacgacctgctgcccccggccccacccagcagtgggggctcgcggacgtgaagggccactggtccccaacaatgtgaggggtccctagcagcccaccctgctgctggtgcacagccactccccggcatgagactcagtgcagatggagagacagctacacagagctttggtctgtgtgtgtgtgtgtgcgtgtgtgtgtgtgtgtgtgcacatccgcgtgtgcctgtgtgcgtgcgcatcttgcctccaggtgcagaggtaccctgggtgtccccgctgctgtgcaacggtctcctgactggtgctgcagcaccgaggggcctttgttctggggggacccagtgcagaatgtaagtgggcccacccggtgggacccccgtggggcagggagctgggcccgacatggctccggcctctgcctttgcaccacgggacatcacagggtgggcctcggcccctcccacacccaaagctgagcctgcagggaagccccacatgtccagcaccttgtgcctggggtgttagtggcaccgcctccccacctccaggctttcccacttcccaccctgcccctcagagactgaaattacgggtacctgaagatgggagcctttaccttttatgcaaaaggtttattccggaaactagtgtacatttctataaatagatgctgtgtatatggtatatatacatatatatatataacatatatggaagaggaaaaggctggtacaacggaggcctgcgaccctgggggcacaggaggcaggcatggccctgggcggggcgtgggggggcgtggagggaggccccagggggtctcacccatgcaagcagaggaccagggccttttctggcaccgcagttttgttttaaaactggacctgtatatttgtaaagctatttatgggcccctggcactcttgttcccacaccccaacacttccagcatttagctggccacatggcggagagttttaatttttaacttattgacaaccgagaaggtttatcccgccgatagagggacggccaagaatgtacgtccagcctgccccggagctggaggatcccctccaagcctaaaaggttgttaatagttggaggtgattccagtgaagatattttatttcctttgtcctttttcaggagaattagatttctataggatttttctttaggagatttattttttggacttcaaagcaagctggtattttcatacaaattcttctaattgctgtgtgtcccaggcagggagacggtttccagggaggggccggccctgtgtgcaggttccgatgttattagatgttacaagtttatatatatctatatatataatttattgagtttttacaagatgtatttgttgtagacttaacacttcttacgcaatgcttctagagttttatagcctggactgctacctttcaaagcttggagggaagccgtgaattcagttggttcgttctgtactgttactgggccctgagtctgggcagctgtcccttgcttgcctgcagggccatggctcagggtggtctcttcttggggcccagtgcatggtggccagaggtgtcacccaaaccggcaggtgcgattttgttaacccagcgacgaactttccgaaaaataaagacacctggttgctaacctggaaaaaaaaaaaaaaaaa

SEQ ID NO: 92 - protein, 838 aa, Homo sapiens
MSLQVLNDKNVSNEKNTENCDFLFSPPEVTGRSSVLRVSQKENVPPKNLAKAMKVTFQTPLRDPQTHRILSPSMASKLEAPFTQDDTLGLENSHPVWTQKENQQLIKEVDAKTTHGILQKPVEADTDLLGDASPAFGSGSSSESGPGALADLDCSSSSQSPGSSENQMVSPGKVSGSPEQAVEENLSSYSLDRRVTPASETLEDPCRTESQHKAETPHGAEEECKAETPHGAEEECRHGGVCAPAAVATSPPGAIPKEACGGAPLQGLPGEALGCPAGVGTPVPADGTQTLTCAHTSAPESTAPTNHLVAGRAMTLSPQEEVAAGQMASSSRSGPVKLEFDVSDGATSKRAPPPRRLGERSGLKPPLRKAAVRQQKAPQEVEEDDGRSGAGEDPPMPASRGSYHLDWDKMDDPNFIPFGGDTKSGCSEAQPPESPETRLGQPAAEQLHAGPATEEPGPCLSQQLHSASAEDTPVVQLAAETPTAESKERALNSASTSLPTSCPGSEPVPTHQQGQPALELKEESFRDPAEVLGTGAEVDYLEQFGTSSFKESALRKQSLYLKFDPLLRDSPGRPVPVATETSSMHGANETPSGRPREAKLVEFDFLGALDIPVPGPPPGVPAPGGPPLSTGPIVDLLQYSQKDLDAVVKATQEENRELRSRCEELHGKNLELGKIMDRFEEVVYQAMEEVQKQKELSKAEIQKVLKEKDQLTTDLNSMEKSFSDLFKRFEKQKEVIEGYRKNEESLKKCVEDYLARITQEGQRYQALKAHAEEKLQLANEEIAQVRSKAQAEALALQASLRKEQMRIQSLEKTVEQKTKENEELTRICDDLISKMEKI

SEQ ID NO: 93 - DNA, 2847 bp, Homo sapiens
  source 1..2847: mol_type = unassigned DNA
gcgtttgaaactccggcgcgccggcggccatcaagggctagaagcgcgacggcggtagcagctaggcttggcccccggcgtggagcagacgcggacccctccttcctggcggcggcggcgcgggctcagagcccggcaacgggcgggcgggcagaatgagtctgcaggtcttaaacgacaaaaatgtcagcaatgaaaaaaatacagaaaattgcgacttcctgttttcgccaccagaagttaccggaagatcgtctgttcttcgtgtgtcacagaaagaaaatgtgccacccaagaacctggccaaagctatgaaggtgacttttcagacacctctgcgggatccacagacgcacaggattctaagtcctagcatggccagcaaacttgaggctcctttcactcaggatgacacccttggactggaaaactcacacccggtctggacacagaaagagaaccaacagctcatcaaggaagtggatgccaaaactactcatggaattctacagaaaccagtggaggctgacaccgacctcctgggggatgcaagcccagcctttgggagtggcagctccagcgagtctggcccaggtgccctggctgacctggactgctcaagctcttcccagagcccaggaagttctgagaaccaaatggtgtctccaggaaaagtgtctggcagccctgagcaagccgtggaggaaaaccttagttcctattccttagacagaagagtgacacccgcctctgagaccctagaagacccttgcaggacagagtcccagcacaaagcggagactccgcacggagccgaggaagaatgcaaagcggagactccgcacggagccgaggaggaatgccggcacggtggggtctgtgctcccgcagcagtggccacttcgcctcctggtgcaatccctaaggaagcctgcggaggagcacccctgcagggtctgcctggcgaagccctgggctgccctgcgggtgtgggcacccccgtgccagcagatggcactcagacccttacctgtgcacacacctctgctcctgagagcacagccccaaccaaccacctggtggctggcagggccatgaccctgagtcctcaggaagaagtggctgcaggccaaatggccagctcctcgaggagcggacctgtaaaactagaatttgatgtatctgatggcgccaccagcaaaagggcacccccaccaaggagactgggagagaggtccggcctcaagcctcccttgaggaaagcagcagtgaggcagcaaaaggccccgcaggaggtggaggaggacgacggtaggagcggagcaggagaggacccccccatgccagcttctcggggctcttaccacctcgactgggacaaaatggatgacccaaacttcatcccgttcggaggtgacaccaagtctggttgcagtgaggcccagcccccagaaagccctgagaccaggctgggccagccagcggctgaacagttgcatgctgggcctgccacggaggagccaggtccctgtctgagccagcagctgcattcagcctcagcggaggacacgcctgtggtgcagttggcagccgagaccccaacagcagagagcaaggagagagccttgaactctgccagcacctcgcttcccacaagctgtccaggcagtgagccagtgcccacccatcagcaggggcagcctgccttggagctgaaagaggagagcttcagagaccccgctgaggttctaggcacgggcgcggaggtggattacctggagcagtttggaacttcctcgtttaaggagtcggccttgaggaagcagtccttatacctcaagttcgaccccctcctgagggacagtcctggtagaccagtgcccgtggccaccgagaccagcagcatgcacggtgcaaatgagactccctcaggacgtccgcgggaagccaagcttgtggagttcgatttcttgggagcactggacattcctgtgccaggcccacccccaggtgttcccgcgcctgggggcccacccctgtccaccggacctatagtggacctgctccagtacagccagaaggacctggatgcagtggtaaaggcgacacaggaggagaaccgggagctgaggagcaggtgtgaggagctccacgggaagaacctggaactggggaagatcatggacaggttcgaagaggttgtgtaccaggccatggaggaagttcagaagcagaaggaactttccaaagctgaaatccagaaagttctaaaagaaaaagaccaacttaccacagatctgaactccatggagaagtccttctccgacctcttcaagcgttttgagaaacagaaagaggtgatcgagggctaccgcaagaacgaagagtcactgaagaagtgcgtggaggattacctggcaaggatcacccaggagggccagaggtaccaagccctgaaggcccacgcggaggagaagctgcagctggcaaacgaggagatcgcccaggtccggagcaaggcccaggcggaagcgttggccctccaggccagcctgaggaaggagcagatgcgcatccagtcgctggagaagacagtggagcagaagactaaagagaacgaggagctgaccaggatctgcgacgacctcatctccaagatggagaagatctgacctccacggagccgctgtccccgcccccctgctcccgtctgtctgtcctgtctgattctcttaggtgtcatgttcttttttctgtcttgtcttcaacttttttaaaaactagattgctttgaaaacatgactcaataaaagtttcctttcaatttaaacactgaaaaaaaaaaaa

SEQ ID NO: 94 - DNA, 3578 bp, Homo sapiens
  source 1..3578: mol_type = unassigned DNA
gtcgcgggcagctggcgccgcgcggtcctgctctgccggtcgcacggacgcaccggcgggccgccggccggagggacggggcgggagctgggcccgcggacagcgagccggagcgggagccgcgcgtagcgagccgggctccggcgctcgccagtctcccgagcggcgcccgcctcccgccggtgcccgcgccgggccgtggggggcagcatgcccgcgcgcgctgcctgaggacgccgcggcccccgcccccgccatgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacaaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgactttaaggagtcggccttgaggaagcagtccttatacctcaagttcgaccccctcctgagggacagtcctggtagaccagtgcccgtggccaccgagaccagcagcatgcacggtgcaaatgagactccctcaggacgtccgcgggaagccaagcttgtggagttcgatttcttgggagcactggacattcctgtgccaggcccacccccaggtgttcccgcgcctgggggcccacccctgtccaccggacctatagtggacctgctccagtacagccagaaggacctggatgcagtggtaaaggcgacacaggaggagaaccgggagctgaggagcaggtgtgaggagctccacgggaagaacctggaactggggaagatcatggacaggttcgaagaggttgtgtaccaggccatggaggaagttcagaagcagaaggaactttccaaagctgaaatccagaaagttctaaaagaaaaagaccaacttaccacagatctgaactccatggagaagtccttctccgacctcttcaagcgttttgagaaacagaaagaggtgatcgagggctaccgcaagaacgaagagtcactgaagaagtgcgtggaggattacctggcaaggatcacccaggagggccagaggtaccaagccctgaaggcccacgcggaggagaagctgcagctggcaaacgaggagatcgcccaggtccggagcaaggcccaggcggaagcgttggccctccaggccagcctgaggaaggagcagatgcgcatccagtcgctggagaagacagtggagcagaagactaaagagaacgaggagctgaccaggatctgcgacgacctcatctccaagatggagaagatctgacctccacggagccgctgtccccgcccccctgctcccgtctgtctgtcctgtctgattctcttaggtgtcatgttcttttttctgtcttgtcttcaacttttttaaaaactagattgctttgaaaacatgactcaataaaagtttcctttcaatttaaacactgaaaaaaaaaaaa

SEQ ID NO: 95 - DNA, 152 bp, synthetic construct
  misc_feature 1..152: note = Description of Artificial Sequence: Synthetic polynucleotide
  source 1..152: mol_type = other DNA
catcctgccccccagagtgctgaggtgtggggcgggccttctggggcacagcctgggcacagaggtggctgtgcgaaggtcgctgagggtccaggcttccacccagtgtccccgcagtcagctgcccaccagcagcctccccgggactctcc

SEQ ID NO: 96 - DNA, 77 bp, synthetic construct
  misc_feature 1..77: note = Description of Artificial Sequence: Synthetic oligonucleotide
  source 1..77: mol_type = other DNA
ggctgggcgaagttcgctgagggtccaggcttcctcccagtgtccccgcagtcagctgcccaccagcagcctccccc

SEQ ID NO: 97 - DNA, 77 bp, synthetic construct
  misc_feature 1..77: note = Description of Artificial Sequence: Synthetic oligonucleotide
  source 1..77: mol_type = other DNA
acagcctgggcacagaggtggctgtgcgaaggtcgctgagggtccaggcttccacccagtgtccccgcagtcagctt

SEQ ID NO: 98 - DNA, 77 bp, synthetic construct
  misc_feature 1..77: note = Description of Artificial Sequence: Synthetic oligonucleotide
  source 1..77: mol_type = other DNA
ggcacagcctgggcacagaggtggctgtgcgaaggtcgctgagggtccaggcttccacccagtgtccccgcagtcaa

SEQ ID NO: 99 - DNA, 77 bp, synthetic construct
  misc_feature 1..77: note = Description of Artificial Sequence: Synthetic oligonucleotide
  source 1..77: mol_type = other DNA
ttctggggcacagcctgggcacagaggtggctgtgcgaaggtcgctgagggtccaggcttccacccagtgtccccgg

SEQ ID NO: 100 - DNA, 77 bp, synthetic construct
  misc_feature 1..77: note = Description of Artificial Sequence: Synthetic oligonucleotide
  source 1..77: mol_type = other DNA
cttctgtggcacagcctggccacagaggtggctgtgcgaaggtcgctgagggtccaggcttccacccagtgtccccc

SEQ ID NO: 101 - DNA, 77 bp, synthetic construct
  misc_feature 1..77: note = Description of Artificial Sequence: Synthetic oligonucleotide
  source 1..77: mol_type = other DNA
gccttctggggcacagcctgggcacagaggtggctgtgcgaaggtcgctgagggtccaggcttccacccagtgtccc

SEQ ID NO: 102 - DNA, 77 bp, synthetic construct
  misc_feature 1..77: note = Description of Artificial Sequence: Synthetic oligonucleotide
  source 1..77: mol_type = other DNA
ggtgtggggcgggccggctggggcacagccggggcacagaggtggctgtgcgaaggtcgctgagggtccaggcttcc

SEQ ID NO: 103 - DNA, 77 bp, synthetic construct
  misc_feature 1..77: note = Description of Artificial Sequence: Synthetic oligonucleotide
  source 1..77: mol_type = other DNA
ggtgtggggcgggccttctggggcacagcctgggcacagaggtggctgtgcgaaggtcgctgagggtccaggcttcc

SEQ ID NO: 104 - DNA, 77 bp, synthetic construct
  misc_feature 1..77: note = Description of Artificial Sequence: Synthetic oligonucleotide
  source 1..77: mol_type = other DNA
tgctgaggtgtggggcgggccttctggggcacagcctgggcacagaggtggctgtgcgaaggtcgctgagggtccaa

SEQ ID NO: 105 - DNA, 77 bp, synthetic construct
  misc_feature 1..77: note = Description of Artificial Sequence: Synthetic oligonucleotide
  source 1..77: mol_type = other DNA
agtgctgaggtgtggggcgggccttctggggcacagcctgggcacagaggtggctgtgcgaaggtcgctgagggtcc

SEQ ID NO: 106 - DNA, 77 bp, synthetic construct
  misc_feature 1..77: note = Description of Artificial Sequence: Synthetic oligonucleotide
  source 1..77: mol_type = other DNA
cccagagtgctgaggtgtggggcgggccttctggggcacagcctgggcacagaggtggctgtgcgaaggtcgctgaa

SEQ ID NO: 107 - DNA, 76 bp, synthetic construct
  misc_feature 1..76: note = Description of Artificial Sequence: Synthetic oligonucleotide
  source 1..76: mol_type = other DNA
cctgccccccagagtgctgaggtgtggggcgggccttctggggcacagcctgggcacagaggtggctgtgcgaagg

SEQ ID NO: 108 - DNA, 153 bp, synthetic construct
  misc_feature 1..153: note = Description of Artificial Sequence: Synthetic polynucleotide
  source 1..153: mol_type = other DNA
gtgctggctctggcctggtgccacccgcctatgcccctccccctgccgtccccggccatcctgccccccagagtgccgggggctaagggccagggaggtcacctgcacactcccacccccggtcacccgcacactcccacccccggtcaccca

SEQ ID NO: 109 - DNA, 76 bp, synthetic construct
  misc_feature 1..76: note = Description of Artificial Sequence: Synthetic oligonucleotide
  source 1..76: mol_type = other DNA
agagtgcagggggctaagggccagggaggtcacctgcacactcccacccccggtcacccgcacactcccacccccg

SEQ ID NO: 110 - DNA, 76 bp, synthetic construct
  misc_feature 1..76: note = Description of Artificial Sequence: Synthetic oligonucleotide
  source 1..76: mol_type = other DNA
ggccatcctgccccccagagtgcagggggctaagggccagggaggtcccctgcacactccctcccccggtcacccg

SEQ ID NO: 111 - DNA, 76 bp, synthetic construct
  misc_feature 1..76: note = Description of Artificial Sequence: Synthetic oligonucleotide
  source 1..76: mol_type = other DNA
tccccctgccgtccccggccatcctgccccccagagtgcagggggctaagggccagggaggtcacctgcacactcc

SEQ ID NO: 112 - DNA, 152 bp, synthetic construct
  misc_feature 1..152: note = Description of Artificial Sequence: Synthetic polynucleotide
  source 1..152: mol_type = other DNA
tgacgtccaccgacgtgagtgctggctctggcctggtgccacccgcctatgcccctccccctgccgtccccggccatccctcaggacgtccgcgggaagccaagcttgtggagttcgatttcttgggagcactggacattcctgtaagtcct

SEQ ID NO: 113 - DNA, 76 bp, synthetic construct
  misc_feature 1..76: note = Description of Artificial Sequence: Synthetic oligonucleotide
  source 1..76: mol_type = other DNA
atccctcaggacgtccgcgggaagccaagcttgtggagttcgatttcttgggagcactggacattcctgtaagtcc

SEQ ID NO: 114 - DNA, 76 bp, synthetic construct
  misc_feature 1..76: note = Description of Artificial Sequence: Synthetic oligonucleotide
  source 1..76: mol_type = other DNA
catccctcaggacgtccgcgggaagccaagcttgtggagttcgatttcttgggagcactggacattcctgtaagtc

SEQ ID NO: 115 - DNA, 76 bp, synthetic construct; identical to SEQ ID NO: 114
  misc_feature 1..76: note = Description of Artificial Sequence: Synthetic oligonucleotide
  source 1..76: mol_type = other DNA

SEQ ID NO: 116 - DNA, 76 bp, synthetic construct
  misc_feature 1..76: note = Description of Artificial Sequence: Synthetic oligonucleotide
  source 1..76: mol_type = other DNA
gccatccctcaggacgtccgcgggaagccaagcttgtggagttcgatttcttgggagcactggacattcctgtaag

SEQ ID NO: 117 - DNA, 76 bp, synthetic construct
  misc_feature 1..76: note = Description of Artificial Sequence: Synthetic oligonucleotide
  source 1..76: mol_type = other DNA
ccggccatccctcaggacgtccgcgggaagccaagcttgtggagttcgatttcttgggagcactggacattcctgt

SEQ ID NO: 118 - DNA, 76 bp, synthetic construct
  misc_feature 1..76: note = Description of Artificial Sequence: Synthetic oligonucleotide
  source 1..76: mol_type = other DNA
cacggccatcccggaggacgtccgcgggaacccaagcttgtggagttcgatttcttggtagcactggacattcctg

SEQ ID NO: 119 - DNA, 76 bp, synthetic construct
  misc_feature 1..76: note = Description of Artificial Sequence: Synthetic oligonucleotide
  source 1..76: mol_type = other DNA
cccggccatccctcaggacgtccgcgggaagccaagcttgtggagttcgatttcttgggagcactggacattcctg

SEQ ID NO: 120 - DNA, 76 bp, synthetic construct
  misc_feature 1..76: note = Description of Artificial Sequence: Synthetic oligonucleotide
  source 1..76: mol_type = other DNA
tccccggccatccctcaggacgtccgcgggaagccaagcttgtggagttcgatttcttgggagcactggacattcc

SEQ ID NO: 121 - DNA, 76 bp, synthetic construct
  misc_feature 1..76: note = Description of Artificial Sequence: Synthetic oligonucleotide
  misc_difference 21: note = modified_base - a, c, t, g, unknown or other
  source 1..76: mol_type = other DNA
tccccggccatccctcaggangtccgcgggaagccaagcttgtggagttcgatttcttgggagcactggacattcc

SEQ ID NO: 122 - DNA, 76 bp, synthetic construct
  misc_feature 1..76: note = Description of Artificial Sequence: Synthetic oligonucleotide
  source 1..76: mol_type = other DNA
gtccccggccatccctcaggacgtccgcgggaagccaagcttgtggagttcgatttcttgggagcactggacattc

SEQ ID NO: 123 - DNA, 76 bp, synthetic construct
  misc_feature 1..76: note = Description of Artificial Sequence: Synthetic oligonucleotide
  source 1..76: mol_type = other DNA
ccgtccccggccatccctcaggacgtccgcgggaagccaagcttgtggagttcgatttcttgggagcactggacat

SEQ ID NO: 124 - DNA, 76 bp, synthetic construct
  misc_feature 1..76: note = Description of Artificial Sequence: Synthetic oligonucleotide
  source 1..76: mol_type = other DNA
tccccctgccgtccccggccatccctcaggacgtccgcgggaagccaagcttgtggagttcgatttcttgggagca

SEQ ID NO: 125 - DNA, 76 bp, synthetic construct
  misc_feature 1..76: note = Description of Artificial Sequence: Synthetic oligonucleotide
  source 1..76: mol_type = other DNA
atgcccctccccctgccgtccccggccatccctcaggacgtccgcgggaagccaagcttgtggagttcgatttctt

SEQ ID NO: 126 - DNA, 76 bp, synthetic construct
  misc_feature 1..76: note = Description of Artificial Sequence: Synthetic oligonucleotide
  source 1..76: mol_type = other DNA
cctatgcccctccccctgccgcccccggccatccctcaggacgtccgcgggaagccaagcttgtggagttcgattt

SEQ ID NO: 127 - DNA, 76 bp, synthetic construct
  misc_feature 1..76: note = Description of Artificial Sequence: Synthetic oligonucleotide
  source 1..76: mol_type = other DNA
ctggcctggtgccacccgcctatgcccctccccctgccgtccccggccatccctcaggacgtccgcgggaagccaa

SEQ ID NO: 128 - DNA, 76 bp, synthetic construct
  misc_feature 1..76: note = Description of Artificial Sequence: Synthetic oligonucleotide
  source 1..76: mol_type = other DNA
gagctggcctggtgccacacgcctatgcccctccccctgccgtccccggcgatccatcaggaagtccgcgggacga

SEQ ID NO: 129 - DNA, 76 bp, synthetic construct
  misc_feature 1..76: note = Description of Artificial Sequence: Synthetic oligonucleotide
  misc_difference 40: note = modified_base - a, c, t, g, unknown or other
  source 1..76: mol_type = other DNA
ggctctggcctggtgccacccgcctatgcccctccccctnccgtccccggccatccctcaggacgtccgcgggaag

SEQ ID NO: 130 - DNA, 76 bp, synthetic construct
  misc_feature 1..76: note = Description of Artificial Sequence: Synthetic oligonucleotide
  source 1..76: mol_type = other DNA
gtgctggctctggcctggtgccacccgcctatgcccctccccctgccgtccccggccatccctcaggacgtccgcg

SEQ ID NO: 131 - DNA, 76 bp, synthetic construct
  misc_feature 1..76: note = Description of Artificial Sequence: Synthetic oligonucleotide
  source 1..76: mol_type = other DNA
ccaccgacgtgagtgctggctctggcctggtgccacccgcctatgcccctccccctgccgtccccggccatccctc

SEQ ID NO: 132 - DNA, 151 bp, synthetic construct
  misc_feature 1..151: note = Description of Artificial Sequence: Synthetic polynucleotide
  source 1..151: mol_type = other DNA
tggaccgtgtccttaccgtgacgtccaccgacgtgagtgctggctctggcctggtgccacccgcctatgcccctcccctggcccttagcccccgtgtgtgttaggggatggcagtcagacctgatcacttgccctcttgtccccagtttaa

SEQ ID NO: 133 - DNA, 80 bp, synthetic construct
  misc_feature 1..80: note = Description of Artificial Sequence: Synthetic oligonucleotide
  source 1..80: mol_type = other DNA
tgcccctcccctgcccttagcccccctgtgtgtgttaggggatggcagtcagacctgatcacttgccctcttgtccgtcc

SEQ ID NO: 134 - DNA, 80 bp, synthetic construct
  misc_feature 1..80: note = Description of Artificial Sequence: Synthetic oligonucleotide
  source 1..80: mol_type = other DNA
atgcccctcccctgcccttagcccccctgtgtgtgttaggggatggcagtcagacctgatcacttgccctcttgtctgtc

SEQ ID NO: 135 - DNA, 80 bp, synthetic construct
  misc_feature 1..80: note = Description of Artificial Sequence: Synthetic oligonucleotide
  source 1..80: mol_type = other DNA
gcctatgcccctcccctgcccttagcccccctgtgtgtgttaggggatggcagtcagacctgatcacttgccctctctct

SEQ ID NO: 136 - DNA, 80 bp, synthetic construct
  misc_feature 1..80: note = Description of Artificial Sequence: Synthetic oligonucleotide
  source 1..80: mol_type = other DNA
gtgccacccgcctatgcccctcccctgcccttagcccccctgtgtgtgttaggggatggcagtcagacctgatcactcac

SEQ ID NO: 137 - DNA, 80 bp, synthetic construct
  misc_feature 1..80: note = Description of Artificial Sequence: Synthetic oligonucleotide
  source 1..80: mol_type = other DNA
gctctggcctggtgccccccgcctatgcccctcccctgcccttagcccccctgtgtgtgttaggggatggcagtcagtca

SEQ ID NO: 138 - DNA, 80 bp, synthetic construct; identical to SEQ ID NO: 137
  misc_feature 1..80: note = Description of Artificial Sequence: Synthetic oligonucleotide
  source 1..80: mol_type = other DNA

SEQ ID NO: 139 - DNA, 81 bp, synthetic construct
  misc_feature 1..81: note = Description of Artificial Sequence: Synthetic oligonucleotide
  source 1..81: mol_type = other DNA
gctgcctctgtcctggtgccccccgcctatgcccctcccctgcccttagcccccctgtgtgtgttaggggatggcatggca

SEQ ID NO: 140 - DNA, 81 bp, synthetic construct
  misc_feature 1..81: note = Description of Artificial Sequence: Synthetic oligonucleotide
  source 1..81: mol_type = other DNA
cacccgccgggggtgcgggctctggcctggtgccccccgcctatgcccctcccctgcccttagcccccctgtgtgttgtgt

SEQ ID NO: 141 - DNA, 80 bp, synthetic construct
  misc_feature 1..80: note = Description of Artificial Sequence: Synthetic oligonucleotide
  source 1..80: mol_type = other DNA
ccccccgacgtgagtgctggctcgggcctggtcccccccgcctatgcccctcccctgcccttagcccccctgtgtgtgtg

SEQ ID NO: 142 - DNA, 82 bp, synthetic construct
  misc_feature 1..82: note = Description of Artificial Sequence: Synthetic oligonucleotide
  source 1..82: mol_type = other DNA
gacgtccaccgacgtgagtgctggctctggcctggtgccacccgcctatgcccctcccctgcccttagaccccctgcccctg

SEQ ID NO: 143 - DNA, 84 bp, synthetic construct
  misc_feature 1..84: note = Description of Artificial Sequence: Synthetic oligonucleotide
  source 1..84: mol_type = other DNA
cttaccgtgacgtccaccgacgtgagtgctggctctggcctggtgccacccgcctatgcccctcccctgcccttaggcccttag

SEQ ID NO: 144 - DNA, 82 bp, synthetic construct
  misc_feature 1..82: note = Description of Artificial Sequence: Synthetic oligonucleotide
  source 1..82: mol_type = other DNA
cccttcaggtccccccccccgacgtgagtgctggctctggcctggtgccacccgcctatgcccctcccctgcccttgccctt

SEQ ID NO: 145 - DNA, 82 bp, synthetic construct
  misc_feature 1..82: note = Description of Artificial Sequence: Synthetic oligonucleotide
  source 1..82: mol_type = other DNA
tgtccttaccgtgacgtccaccgacgtgagtgctggctctggcctggtgccacccgcctatgcccctcccctgcccctgccc

SEQ ID NO: 146 - protein, 820 aa, Homo sapiens
MWSWKCLLFWAVLVTATLCTARPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNRMPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPLYLEIIIYCTGAFLISCMVGSVIVYKMKSGTKKSDFHSQMAVHKLAKSIPLRRQVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSMPLDQYSPSFPDTRSSTCSSGEDSVFSHEPLPEEPCLPRHPAQLANGGLKRR

SEQ ID NO: 147 - DNA, 5895 bp, Homo sapiens
  source 1..5895: mol_type = unassigned DNA
agatgcaggggcgcaaacgccaaaggagaccaggctgtaggaagagaagggcagagcgccggacagctcggcccgctccccgtcctttggggccgcggctggggaactacaaggcccagcaggcagctgcagggggcggaggcggaggagggaccagcgcgggtgggagtgagagagcgagccctcgcgccccgccggcgcatagcgctcggagcgctcttgcggccacaggcgcggcgtcctcggcggcgggcggcagctagcgggagccgggacgccggtgcagccgcagcgcgcggaggaacccgggtgtgccgggagctgggcggccacgtccggacgggaccgagacccctcgtagcgcattgcggcgacctcgccttccccggccgcgagcgcgccgctgcttgaaaagccgcggaacccaaggacttttctccggtccgagctcggggcgccccgcagggcgcacggtacccgtgctgcagtcgggcacgccgcggcgccggggcctccgcagggcgatggagcccggtctgcaaggaaagtgaggcgccgccgctgcgttctggaggaggggggcacaaggtctggagaccccgggtggcggacgggagccctccccccgccccgcctccggggcaccagctccggctccattgttcccgcccgggctggaggcgccgagcaccgagcgccgccgggagtcgagcgccggccgcggagctcttgcgaccccgccaggacccgaacagagcccgggggcggcgggccggagccggggacgcgggcacacgcccgctcgcacaagccacggcggactctcccgaggcggaacctccacgccgagcgagggtcagtttgaaaaggaggatcgagctcactgtggagtatccatggagatgtggagccttgtcaccaacctctaactgcagaactgggatgtggagctggaagtgcctcctcttctgggctgtgctggtcacagccacactctgcaccgctaggccgtccccgaccttgcctgaacaagcccagccctggggagcccctgtggaagtggagtccttcctggtccaccccggtgacctgctgcagcttcgctgtcggctgcgggacgatgtgcagagcatcaactggctgcgggacggggtgcagctggcggaaagcaaccgcacccgcatcacaggggaggaggtggaggtgcaggactccgtgcccgcagactccggcctctatgcttgcgtaaccagcagcccctcgggcagtgacaccacctacttctccgtcaatgtttcagatgctctcccctcctcggaggatgatgatgatgatgatgactcctcttcagaggagaaagaaacagataacaccaaaccaaaccgtatgcccgtagctccatattggacatccccagaaaagatggaaaagaaattgcatgcagtgccggctgccaagacagtgaagttcaaatgcccttccagtgggaccccaaaccccacactgcgctggttgaaaaatggcaaagaattcaaacctgaccacagaattggaggctacaaggtccgttatgccacctggagcatcataatggactctgtggtgccctctgacaagggcaactacacctgcattgtggagaatgagtacggcagcatcaaccacacataccagctggatgtcgtggagcggtcccctcaccggcccatcctgcaagcagggttgcccgccaacaaaacagtggccctgggtagcaacgtggagttcatgtgtaaggtgtacagtgacccgcagccgcacatccagtggctaaagcacatcgaggtgaatgggagcaagattggcccagacaacctgccttatgtccagatcttgaagactgctggagttaataccaccgacaaagagatggaggtgcttcacttaagaaatgtctcctttgaggacgcaggggagtatacgtgcttggcgggtaactctatcggactctcccatcactctgcatggttgaccgttctggaagccctggaagagaggccggcagtgatgacctcgcccctgtacctggagatcatcatctattgcacaggggccttcctcatctcctgcatggtggggtcggtcatcgtctacaagatgaagagtggtaccaagaagagtgacttccacagccagatggctgtgcacaagctggccaagagcatccctctgcgcagacaggtgtctgctgactccagtgcatccatgaactctggggttcttctggttcggccatcacggctctcctccagtgggactcccatgctagcaggggtctctgagtatgagcttcccgaagaccctcgctgggagctgcctcgggacagactggtcttaggcaaacccctgggagagggctgctttgggcaggtggtgttggcagaggctatcgggctggacaaggacaaacccaaccgtgtgaccaaagtggctgtgaagatgttgaagtcggacgcaacagagaaagacttgtcagacctgatctcagaaatggagatgatgaagatgatcgggaagcataagaatatcatcaacctgctgggggcctgcacgcaggatggtcccttgtatgtcatcgtggagtatgcctccaagggcaacctgcgggagtacctgcaggcccggaggcccccagggctggaatactgctacaaccccagccacaacccagaggagcagctctcctccaaggacctggtgtcctgcgcctaccaggtggcccgaggcatggagtatctggcctccaagaagtgcatacaccgagacctggcagccaggaatgtcctggtgacagaggacaatgtgatgaagatagcagactttggcctcgcacgggacattcaccacatcgactactataaaaagacaaccaacggccgactgcctgtgaagtggatggcacccgaggcattatttgaccggatctacacccaccagagtgatgtgtggtctttcggggtgctcctgtgggagatcttcactctgggcggctccccataccccggtgtgcctgtggaggaacttttcaagctgctgaaggagggtcaccgcatggacaagcccagtaactgcaccaacgagctgtacatgatgatgcgggactgctggcatgcagtgccctcacagagacccaccttcaagcagctggtggaagacctggaccgcatcgtggccttgacctccaaccaggagtacctggacctgtccatgcccctggaccagtactcccccagctttcccgacacccggagctctacgtgctcctcaggggaggattccgtcttctctcatgagccgctgcccgaggagccctgcctgccccgacacccagcccagcttgccaatggcggactcaaacgccgctgactgccacccacacgccctccccagactccaccgtcagctgtaaccctcacccacagcccctgctgggcccaccacctgtccgtccctgtcccctttcctgctggcaggagccggctgcctaccaggggccttcctgtgtggcctgccttcaccccactcagctcacctctccctccacctcctctccacctgctggtgagaggtgcaaagaggcagatctttgctgccagccacttcatcccctcccagatgttggaccaacacccctccctgccaccaggcactgcctggagggcagggagtgggagccaatgaacaggcatgcaagtgagagcttcctgagctttctcctgtcggtttggtctgttttgccttcacccataagcccctcgcactctggtggcaggtgccttgtcctcagggctacagcagtagggaggtcagtgcttcgtgcctcgattgaaggtgacctctgccccagataggtggtgccagtggcttattaattccgatactagtttgctttgctgaccaaatgcctggtaccagaggatggtgaggcgaaggccaggttgggggcagtgttgtggccctggggcccagccccaaactgggggctctgtatatagctatgaagaaaacacaaagtgtataaatctgagtatatatttacatgtctttttaaaagggtcgttaccagagatttacccatcgggtaagatgctcctggtggctgggaggcatcagttgctatatattaaaaacaaaaaagaaaaaaaaggaaaatgtttttaaaaaggtcatatattttttgctacttttgctgttttatttttttaaattatgttctaaacctattttcagtttaggtccctcaataaaaattgctgctgcttcatttatctatgggctgtatgaaaagggtgggaatgtccactggaaagaagggacacccacgggccctggggctaggtctgtcccgagggcaccgcatgctcccggcgcaggttccttgtaacctcttcttcctaggtcctgcacccagacctcacgacgcacctcctgcctctccgctgcttttggaaagtcagaaaaagaagatgtctgcttcgagggcaggaaccccatccatgcagtagaggcgctgggcagagagtcaaggcccagcagccatcgaccatggatggtttcctccaaggaaaccggtggggttgggctggggagggggcacctacctaggaatagccacggggtagagctacagtgattaagaggaaagcaagggcgcggttgctcacgcctgtaatcccagcactttgggacaccgaggtgggcagatcacttcaggtcaggagtttgagaccagcctggccaacttagtgaaaccccatctctactaaaaatgcaaaaattatccaggcatggtggcacacgcctgtaatcccagctccacaggaggctgaggcagaatcccttgaagctgggaggcggaggttgcagtgagccgagattgcgccattgcactccagcctgggcaacagagaaaacaaaaaggaaaacaaatgatgaaggtctgcagaaactgaaacccagacatgtgtctgccccctctatgtgggcatggttttgccagtgcttctaagtgcaggagaacatgtcacctgaggctagttttgcattcaggtccctggcttcgtttcttgttggtatgcctccccagatcgtccttcctgtatccatgtgaccagactgtatttgttgggactgtcgcagatcttggcttcttacagttcttcctgtccaaactccatcctgtccctcaggaacggggggaaaattctccgaatgtttttggttttttggctgcttggaatttacttctgccacctgctggtcatcactgtcctcactaagtggattctggctcccccgtacctcatggctcaaactaccactcctcagtcgctatattaaagcttatattttgctggattactgctaaatacaaaagaaagttcaatatgttttcatttctgtagggaaaatgggattgctgctttaaatttctgagctagggattttttggcagctgcagtgttggcgactattgtaaaattctctttgtttctctctgtaaatagcacctgctaacattacaatttgtatttatgtttaaagaaggcatcatttggtgaacagaactaggaaatgaatttttagctcttaaaagcatttgctttgagaccgcacaggagtgtctttccttgtaaaacagtgatgataatttctgccttggccctaccttgaagcaatgttgtgtgaagggatgaagaatctaaaagtcttcataagtccttgggagaggtgctagaaaaatataaggcactatcataattacagtgatgtccttgctgttactactcaaatcacccacaaatttccccaaagactgcgctagctgtcaaataaaagacagtgaaattgacctga

SEQ ID NO: 148 - protein, 805 aa, Homo sapiens
MAFSPWQILSPVQWAKWTWSAVRGGAAGEDEAGGPEGDPEEEDSQAETKSLSFSSDSEGNFETPEAETPIRSPFKESCDPSLGLAGPGAKSQESQEADEQLVAEVVEKCSSKTCSKPSENEVPQQAIDSHSVKNFREEPEHDFSKISIVRPFSIETKDSTDISAVLGTKAAHGCVTAVSGKALPSSPPDALQDEAMTEGSMGVTLEASAEADLKAGNSCPELVPSRRSKLRKPKPVPLRKKAIGGEFSDTNAAVEGTPLPKASYHFSPEELDENTSPLLGDARFQKSPPDLKETPGTLSSDTNDSGVELGEESRSSPLKLEFDFTEDTGNIEARKALPRKLGRKLGSTLTPKIQKDGISKSAGLEQPTDPVARDGPLSQTSSKPDPSQWESPSFNPFGSHSVLQNSPPLSSEGSYHFDPDNFDESMDPFKPTTTLTSSDFCSPTGNHVNEILESPKKAKSRLITSGCKVKKHETQSLALDACSRDEGAVISQISDISNRDGHATDEEKLASTSCGQKSAGAEVKGEPEEDLEYFECSNVPVSTINHAFSSSEAGIEKETCQKMEEDGSTVLGLLESSAEKAPVSVSCGGESPLDGICLSESDKTAVLTLIREEIITKEIEANEWKKKYEETRQEVLEMRKIVAEYEKTIAQMIEDEQRTSMTSQKSFQQLTMEKEQALADLNSVERSLSDLFRRYENLKGVLEGFKKNEEALKKCAQDYLARVKQEEQRYQALKIHAEEKLDKANEEIAQVRTKAKAESAALHAGLRKEQMKVESLERALQQKNQEIEELTKICDELIAKLGKTD

SEQ ID NO: 149 - DNA, 7802 bp, Homo sapiens
  source 1..7802: mol_type = unassigned DNA
agctgatgcgcgccccgccggccgggaggcgggagtccgcgagccgggagcgggagcagcagaggtctagcagccgggcgccgcgggccgggggcctgaggaggccacaggacgggcgtcttcccggctagtggagcccggcgcggggcccgctgcggccgcaccgtgaggggaggaggccgaggaggacgcagcgccggctgccggcgggaggaagcgctccaccagggcccccgacggcactcgtttaaccacatccgcgcctctgctggaaacgcttgctggcgcctgtcaccggttccctccattttgaaagggaaaaaggctctccccacccattcccctgcccctaggagctggagccggaggagccgcgctcatggcgttcagcccgtggcagatcctgtcccccgtgcagtgggcgaaatggacgtggtctgcggtacgcggcggggccgccggcgaggacgaggctggcgggcccgagggcgaccccgaggaggaggattcgcaagccgagaccaaatccttgagtttcagctcggattctgaaggtaattttgagactcctgaagctgaaaccccgatccgatcacctttcaaggagtcctgtgatccatcactcggattggcaggacctggggccaaaagccaagaatcacaagaagctgatgaacagcttgtagcagaagtggttgaaaaatgttcatctaagacttgttctaaaccttcagaaaatgaagtgccacagcaggccattgactctcactcagtcaagaatttcagagaagaacctgaacatgattttagcaaaatttccatcgtgaggccattttcaatagaaacgaaggattccacggatatctcggcagtcctcggaacaaaagcagctcatggctgtgtaactgcagtctcaggcaaggctctgccttccagcccgccagacgccctccaggacgaggcgatgacagaaggcagcatgggggtcaccctcgaggcctccgcagaagctgatctaaaagctggcaactcctgtccagagcttgtgcccagcagaagaagcaagctgagaaagcccaagcctgtccccctgaggaagaaagcaattggaggagagttctcagacaccaacgctgctgtggagggcacacctctccccaaggcatcctatcacttcagtcctgaagagttggatgagaacacaagtcctttgctaggagatgccaggttccagaagtctccccctgaccttaaagaaactcccggcactctcagtagtgacaccaacgactcaggggttgagctgggggaggagtcgaggagctcacctctcaagcttgagtttgatttcacagaagatacaggaaacatagaggccaggaaagcccttccaaggaagcttggcaggaaactgggtagcacactgactcccaagatacaaaaagatggcatcagtaagtcagcaggtttagaacagcctacagacccagtggcacgagacgggcctctctcccaaacatcttccaagccagatcctagtcagtgggaaagccccagcttcaacccctttgggagccactctgttctgcagaactccccacccctctcttctgagggctcctaccactttgacccagataactttgacgaatccatggatccctttaaaccaactacgaccttaacaagcagtgacttttgttctcccactggtaatcacgttaatgaaatcttagaatcacccaagaaggcaaagtcgcgtttaataacgagtggctgtaaggtgaagaagcatgaaactcagtctctcgccctggatgcatgttctcgggatgaaggggcagtgatctcccagatttcagacatttctaatagggatggccatgctactgatgaggagaaactggcatccacgtcatgtggtcagaaatcagctggtgccgaggtgaaaggtgagccagaggaagacctggagtactttgaatgttccaatgttcctgtgtctaccataaatcatgcgttttcatcctcagaagcaggcatagagaaggagacgtgccagaagatggaagaagacgggtccactgtgcttgggctgctggagtcctctgcagagaaggcccctgtgtcggtgtcctgtggaggtgagagccccctggatgggatctgcctcagcgaatcagacaagacagccgtgctcaccttaataagagaagagataattactaaagagattgaagcaaatgaatggaagaagaaatacgaagagacccggcaagaagttttggagatgaggaaaattgtagctgaatatgaaaagactattgctcaaatgattgaagatgaacaaaggacaagtatgacctctcagaagagcttccagcaactgaccatggagaaggaacaggccctggctgaccttaactctgtggaaaggtccctttctgatctcttcaggagatatgagaacctgaaaggtgttctggaagggttcaagaagaatgaagaagccttgaagaaatgtgctcaggattacttagccagagttaaacaagaggagcagcgataccaggccctgaaaatccacgcagaagagaaactggacaaagccaatgaagagattgctcaggttcgaacaaaagcaaaggctgagagtgcagctctccatgctggactccgcaaagagcagatgaaggtggagtccctggaaagggccctgcagcagaagaaccaagaaattgaagaactgacaaaaatctgtgatgagctgattgcaaagctgggaaagactgactgagacactccccctgttagctcaacagatctgcatttggctgcttctcttgtgaccacaattatcttgccttatccaggaataattgcccctttgcagagaaaaaaaaaaacttaaaaaaagcacatgcctactgctgcctgtcccgctttgctgccaatgcaacagccctggaagaaaccctagagggttgcatagtctagaaaggagtgtgacctgacagtgctggagcctcctagtttccccctatgaaggttcccttaggctgctgagtttgggtttgtgatttatctttagtttgttttaaagtcatctttactttcccaaatgtgttaaatttgtaactcctctttggggtcttctccaccacctgtctgatttttttgtgatctgtttaatcttttaattttttagtatcagtggttttatttaaggagacagtttggcctattgttacttccaatttataatcaagaaggggctctggatccccttttaaattacacacactctcacacacatacatgtatgtttatagatgctgctgctcttttccctgaagcatagtcaagtaagaactgctctacagaaggacatatttccttggatgtgagaccctattttgaaatagagtcctgactcagaacaccaacttaagaatttgggggattaaagatgtgaagaccacagtcttgggttttcatatctggagaagactatttgccatgacgttttgttgccctggtatttggacactcctcagctttaatgggtgtggcccctttagggttagtcctcagactaatgatagtgtctgctttctgcatgaacggcaatatgggactccctccaagctagggtttggcaagtctgccctagagtcatttactctcctctgcctccatttgttaatacagaatcaacatttagtcttcattatctttttttttttttttgagacagagtttcgatctattttaagtatgtgaagaaaatctacttgtaaaaggctcagatcttaattaaaaggtaattgtagcacattaccaattataaggtgaagaaatgtttttttcccaagtgtgatgcattgttcttcagatgttgaaaagaaagcaaaaaataccttctaacttaagacagaatttttaacaaaatgagcagtaaaagtcacatgaaccactccaaaaatcagtgcattttgcatatttttaaacaaagacagcttgttgaatactgagaagaggagtgcaaggagaaggtctgtactaacaaagccaaattcctcaagctcttactggactcagttcagagtggtgggccattaaccccaacatggaatttttccatataaatctcaatgaattccctttcatttgaataggcaaacccaaatccatgcaagtgttttaaagcactgtcctgtcttaatcttacatgctgaaagtcttcatggtgatatgcactatattcagtatacgtatgttttcctacttctcttgtaaaactgttgcatgatccaacttcagcaatgaattgtgcctagtggagaacctctatagatcttaaaaaatgaattattctttagcagtgtattactcacatgggtgcaatctttagccccagggaggtcaataatgtcttttaaagccagaagtcacattttaccaatatgcatttatcataattggtgcttaggctgtatattcaagcctgttgtcttaacattttgtataaaaaagaacaacagaaattatctgtcatttgagaagtggcttgacaatcatttgagctttgaaagcagtcactgtggtgtaatatgaatgctgtcctagtggtcatagtaccaagggcacgtgtctccccttggtataactgatttcctttttagtcctctactgctaaataagttaattttgcattttgcagaaagaaacattgattgctaaatctttttgctgctgtgttttggtgttttcatgtttacttgttttatattgatctgttttaagtatgagaggcttatagtgccctccattgtaaatccatagtcatctttttaagcttattgtgtttaagaaagtagctatgtgttaaacagaggtgatggcagcccttccctagcacactggtggaagagaccccttaagaacctgaccccagtgaatgaagctgatgcacagggagcaccaaaggaccttcgttaagtgataattgtcctggcctctcagccatgaccgttatgaggaaatatcccccattcgaacttaacagatgcctcctctccaaagagaattaaaatcgtagcttgtacagatcaagagaatatactgggcagaatgaagtatgtttgtttatttttctttaaaaataaaggattttggaactctggagagtaagaatatagtatagagtttgcctcaacacatgtgagggccaaataacctgctagctaggcagtaataaactctgttacagaagagaaaaagggccgggcacagtggcttattcctgtaatcccaacactgtggaaggccgaggcaggaggatcacttgagtccaggagtttgaaacctacctaggcaacatggtgaaaccttgtctctaccaaaataaaaattagctgggcatggtggcacgtgcctgtggtcccagctacttgggaggctgaggtgggagcctgggaggtcaaggctgcagtgagccatgatcatgccactgcactccatcctgggtgacagcaagatcttgtctcaaaaaaaaaaaaaaaaaaaaaaaaaccaggagtgaaaaaggaaagtagaaggcagctgctggcctagatgttggtttgggaatattaggtgatcctgttgagattctggatccagagcaatttctttagcttttgactttgccaaagtgtagatagcctttatccagcagtattttaagtggggaatgcaacgtgaggccaactgaacaattccccccgtggctgcccagatagtcacagtcaaggttggagagtctccttccagccagtgacctacccaaaccttttgttctgtaaaactgctctggaaataccgggaagcccagttttctcacgtggtttctagcttcttcagactcagcccaaattaggaagtgcagaagcacatgatggtgaaaaacctaggatttggcagccttccagaatggtatggaatctgagggaagatttatgtttcgttttggaggatagctcaagttgaattttctttccagccagttaccctttcaacctacccatactttgtacaactcttacacaaatacttagatatttattagatagccctgaattcactctaattataaacagggagtgtaaactgcccccagatgttcctgggctgggtaaaagcagctggagtgaagcactcattttccataaaggtaacaaagggcagctcagtggttactcaagctcaaaagggtttttttaagagcaagcattggttaagtctgtgtatactgagttggaagtgatttcagcacattcttttttagtggagtgaaagttctgaagcccccttttaacttcctcttggtttttcattataattggtagccatctcatgaactgtctctgactgttgtctctttgtggtcatgtgattgtgagcttgctttctgacttgcatttctgactttatcctgttgttaggaagatagaaactaggttttgaaagattacatgattcaagcgagggattttaaagtaaagatgtatttattctgaagaatctaaaagataacagattatttgcttatgaaagaacaatatagtctgggaatcccagaatgtcaagccaaaggtctaagaagtcatctccttcaaatactttaataaagaagtatttcgaggagatatctgtccaaaaaggtttgactggcctccagattccagttatttttaaaaagcaacttaccactaaatccttgagtctccatagagtaacagtaaagaaactgatgtaacagactctcctctcaaaggatctcctctggaagagactatcagcggcagcattctccagggaagacccatcccctagtgccagagcttgcatcctggagactaaagattgcacttttttgtagttttttgtccaaatgcaatcccatttctgtgcctcttagcatgcagttagatttggacaaacaagattcctaaggaatgactttattaactataatatggttacagctattatataaatatatattctggttatagttctaatatggagatgttgtgtgcaatgctggcctgtggtggtctgtgtaatgctttaacttgtatggaggaggccaggctcagagctgagatgtggcctgaaccttccctgtatcgatcctttaatttagaactgtcaagatgtcactttctccccctctgccttttagtggtatctgacatatactcaaaacagtaatttcctggtcacatcattaactgctaattctgtatttataaagaattttcagatggacatgtacaaatttgaactcaaaccatccccagtccagatacagggcagcgtgtaggtgaccacaccagagcctcagcctcggtccttctcagccgtcgggataggatccaggcatttcttttaaatctcagaggtagcagtaaacttttcagtattgctgttagcaagtgtgtgtttgccaatagatacccattatactaatgtgccaagtaaatgttcattgcacatctgcttccactgtgttcccacgggtgccatgaagtgtgtgaggagcccctcatctggagggatgagtgctgcgttgactactgctatcaggattgtgttgtgtggaatattcatctacataaattttatatgcacagtaatttccctttttatatgtcaagtaactatttgtaaaagttatactcacaaattattataatgattactaatatattttttccatgtttcattgcctgaataaaaactgtttaccactgtta

SEQ ID NO: 150 - protein, 907 aa, synthetic construct
  REGION 1..907: note = Description of Artificial Sequence: Synthetic polypeptide
MWSWKCLLFWAVLVTATLCTARPSPTLPEQDALPSSEDDDDDDDSSSEEKETDNTKPNPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPLYLEIIIYCTGAFLISCMVGSVIVYKMKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQGLLESSAEKAPVSVSCGGESPLDGICLSESDKTAVLTLIREEIITKEIEANEWKKKYEETRQEVLEMRKIVAEYEKTIAQMIEDEQRTSMTSQKSFQQLTMEKEQALADLNSVERSLSDLFRRYENLKGVLEGFKKNEEALKKCAQDYLARVKQEEQRYQALKIHAEEKLDKANEEIAQVRTKAKAESAALHAGLRKEQMKVESLERALQQKNQEIEELTKICDELIAKLGKTD

SEQ ID NO: 151 - DNA, 2989 bp, synthetic construct
  misc_feature 1..2989: note = Description of Artificial Sequence: Synthetic polynucleotide
  source 1..2989: mol_type = other DNA
atgtggagctggaagtgcctcctcttctgggctgtgctggtcacagccacactctgcaccgctaggccgtccccgaccttgcctgaacaagcccagccctggggagcccctgtggaagtggagtccttcctggtccaccccggtgacctgctgcagcttcgctgtcggctgcgggacgatgtgcagagcatcaactggctgcgggacggggtgcagctggcggaaagcaaccgcacccgcatcacaggggaggaggtggaggtgcaggactccgtgcccgcagactccggcctctatgcttgcgtaaccagcagcccctcgggcagtgacaccacctacttctccgtcaatgtttcagatgctctcccctcctcggaggatgatgatgatgatgatgactcctcttcagaggagaaagaaacagataacaccaaaccaaaccgtatgcccgtagctccatattggacatccccagaaaagatggaaaagaaattgcatgcagtgccggctgccaagacagtgaagttcaaatgcccttccagtgggaccccaaaccccacactgcgctggttgaaaaatggcaaagaattcaaacctgaccacagaattggaggctacaaggtccgttatgccacctggagcatcataatggactctgtggtgccctctgacaagggcaactacacctgcattgtggagaatgagtacggcagcatcaaccacacataccagctggatgtcgtggagcggtcccctcaccggcccatcctgcaagcagggttgcccgccaacaaaacagtggccctgggtagcaacgtggagttcatgtgtaaggtgtacagtgacccgcagccgcacatccagtggctaaagcacatcgaggtgaatgggagcaagattggcccagacaacctgccttatgtccagatcttgaagactgctggagttaataccaccgacaaagagatggaggtgcttcacttaagaaatgtctcctttgaggacgcaggggagtatacgtgcttggcgggtaactctatcggactctcccatcactctgcatggttgaccgttctggaagccctggaagagaggccggcagtgatgacctcgcccctgtacctggagatcatcatctattgcacaggggccttcctcatctcctgcatggtggggtcggtcatcgtctacaagatgaagagtggtaccaagaagagtgacttccacagccagatggctgtgcacaagctggccaagagcatccctctgcgcagacaggtgtctgctgactccagtgcatccatgaactctggggttcttctggttcggccatcacggctctcctccagtgggactcccatgctagcaggggtctctgagtatgagcttcccgaagaccctcgctgggagctgcctcgggacagactggtcttaggcaaacccctgggagagggctgctttgggcaggtggtgttggcagaggctatcgggctggacaaggacaaacccaaccgtgtgaccaaagtggctgtgaagatgttgaagtcggacgcaacagagaaagacttgtcagacctgatctcagaaatggagatgatgaagatgatcgggaagcataagaatatcatcaacctgctgggggcctgcacgcaggatggtcccttgtatgtcatcgtggagtatgcctccaagggcaacctgcgggagtacctgcaggcccggaggcccccagggctggaatactgctacaaccccagccacaacccagaggagcagctctcctccaaggacctggtgtcctgcgcctaccaggtggcccgaggcatggagtatctggcctccaagaagtgcatacaccgagacctggcagccaggaatgtcctggtgacagaggacaatgtgatgaagatagcagactttggcctcgcacgggacattcaccacatcgactactataaaaagacaaccaacggccgactgcctgtgaagtggatggcacccgaggcattatttgaccggatctacacccaccagagtgatgtgtggtctttcggggtgctcctgtgggagatcttcactctgggcggctccccataccccggtgtgcctgtggaggaacttttcaagctgctgaaggagggtcaccgcatggacaagcccagtaactgcaccaacgagctgtacatgatgatgcgggactgctggcatgcagtgccctcacagagacccaccttcaagcagctggtggaagacctggaccgcatcgtggccttgacctccaaccagtgggctgctggagtcctctgcagagaaggcccctgtgtcggtgtcctgtggaggtgagagccccctggatgggatctgcctcagcgaatcagacaagacagccgtgctcaccttaataagagaagagataattactaaagagattgaagcaaatgaatggaagaagaaatacgaagagacccggcaagaagttttggagatgaggaaaattgtagctgaatatgaaaagactattgctcaaatgattgaagatgaacaaaggacaagtatgacctctcagaagagcttccagcaactgaccatggagaaggaacaggccctggctgaccttaactctgtggaaaggtccctttctgatctcttcaggagatatgagaacctgaaaggtgttctggaagggttcaagaagaatgaagaagccttgaagaaatgtgctcaggattacttagccagagttaaacaagaggagcagcgataccaggccctgaaaatccacgcagaagagaaactggacaaagccaatgaagagattgctcaggttcgaacaaaagcaaaggctgagagtgcagctctccatgctggactccgcaaagagcagatgaaggtggagtccctggaaagggccctgcagcagaagaaccaagaaattgaagaactgacaaaaatctgtgatgagctgattgcaaagctgggaaagactgac

SEQ ID NO: 152 - protein, 821 aa, Homo sapiens
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEEYLDLSQPLEQYSPSYPDTRSSCSSGDDSVFSPDPMPYEPCLPQYPHINGSVKT

SEQ ID NO: 153 - DNA, 4654 bp, Homo sapiens
  source 1..4654: mol_type = unassigned DNA
ggcggcggctggaggagagcgcggtggagagccgagcgggcgggcggcgggtgcggagcgggcgagggagcgcgcgcggccgccacaaagctcgggcgccgcggggctgcatgcggcgtacctggcccggcgcggcgactgctctccgggctggcgggggccggccgcgagccccgggggccccgaggccgcagcttgcctgcgcgctctgagccttcgcaactcgcgagcaaagtttggtggaggcaacgccaagcctgagtcctttcttcctctcgttccccaaatccgagggcagcccgcgggcgtcatgcccgcgctcctccgcagcctggggtacgcgtgaagcccgggaggcttggcgccggcgaagacccaaggaccactcttctgcgtttggagttgctccccgcaaccccgggctcgtcgctttctccatcccgacccacgcggggcgcggggacaacacaggtcgcggaggagcgttgccattcaagtgactgcagcagcagcggcagcgcctcggttcctgagcccaccgcaggctgaaggcattgcgcgtagtccatgcccgtagaggaagtgtgcagatgggattaacgtccacatggagatatggaagaggaccggggattggtaccgtaaccatggtcagctggggtcgtttcatctgcctggtcgtggtcaccatggcaaccttgtccctggcccggccctccttcagtttagttgaggataccacattagagccagaagagccaccaaccaaataccaaatctctcaaccagaagtgtacgtggctgcgccaggggagtcgctagaggtgcgctgcctgttgaaagatgccgccgtgatcagttggactaaggatggggtgcacttggggcccaacaataggacagtgcttattggggagtacttgcagataaagggcgccacgcctagagactccggcctctatgcttgtactgccagtaggactgtagacagtgaaacttggtacttcatggtgaatgtcacagatgccatctcatccggagatgatgaggatgacaccgatggtgcggaagattttgtcagtgagaacagtaacaacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtagtggagaatgaatacgggtccatcaatcacacgtaccacctggatgttgtggagcgatcgcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctggagatagccatttactgcataggggtcttcttaatcgcctgtatggtggtaacagtcatcctgtgccgaatgaagaacacgaccaagaagccagacttcagcagccagccggctgtgcacaagctgaccaaacgtatccccctgcggagacaggtaacagtttcggctgagtccagctcctccatgaactccaacaccccgctggtgaggataacaacacgcctctcttcaacggcagacacccccatgctggcaggggtctccgagtatgaacttccagaggacccaaaatgggagtttccaagagataagctgacactgggcaagcccctgggagaaggttgctttgggcaagtggtcatggcggaagcagtgggaattgacaaagacaagcccaaggaggcggtcaccgtggccgtgaagatgttgaaagatgatgccacagagaaagacctttctgatctggtgtcagagatggagatgatgaagatgattgggaaacacaagaatatcataaatcttcttggagcctgcacacaggatgggcctctctatgtcatagttgagtatgcctctaaaggcaacctccgagaatacctccgagcccggaggccacccgggatggagtactcctatgacattaaccgtgttcctgaggagcagatgaccttcaaggacttggtgtcatgcacctaccagctggccagaggcatggagtacttggcttcccaaaaatgtattcatcgagatttagcagccagaaatgttttggtaacagaaaacaatgtgatgaaaatagcagactttggactcgccagagatatcaacaatatagactattacaaaaagaccaccaatgggcggcttccagtcaagtggatggctccagaagccctgtttgatagagtatacactcatcagagtgatgtctggtccttcggggtgttaatgtgggagatcttcactttagggggctcgccctacccagggattcccgtggaggaactttttaagctgctgaaggaaggacacagaatggataagccagccaactgcaccaacgaactgtacatgatgatgagggactgttggcatgcagtgccctcccagagaccaacgttcaagcagttggtagaagacttggatcgaattctcactctcacaaccaatgaggaatacttggacctcagccaacctctcgaacagtattcacctagttaccctgacacaagaagttcttgttcttcaggagatgattctgttttttctccagaccccatgccttacgaaccatgccttcctcagtatccacacataaacggcagtgttaaaacatgaatgactgtgtctgcctgtccccaaacaggacagcactgggaacctagctacactgagcagggagaccatgcctcccagagcttgttgtctccacttgtatatatggatcagaggagtaaataattggaaaagtaatcagcatatgtgtaaagatttatacagttgaaaacttgtaatcttccccaggaggagaagaaggtttctggagcagtggactgccacaagccaccatgtaacccctctcacctgccgtgcgtactggctgtggaccagtaggactcaaggtggacgtgcgttctgccttccttgttaattttgtaataattggagaagatttatgtcagcacacacttacagagcacaaatgcagtatataggtgctggatgtatgtaaatatattcaaattatgtataaatatatattatatatttacaaggagttattttttgtattgattttaaatggatgtcccaatgcacctagaaaattggtctctctttttttaatagctatttgctaaatgctgttcttacacataatttcttaattttcaccgagcagaggtggaaaaatacttttgctttcagggaaaatggtataacgttaatttattaataaattggtaatatacaaaacaattaatcatttatagttttttttgtaatttaagtggcatttctatgcaggcagcacagcagactagttaatctattgcttggacttaactagttatcagatcctttgaaaagagaatatttacaatatatgactaatttggggaaaatgaagttttgatttatttgtgtttaaatgctgctgtcagacgattgttcttagacctcctaaatgccccatattaaaagaactcattcataggaaggtgtttcattttggtgtgcaaccctgtcattacgtcaacgcaacgtctaactggacttcccaagataaatggtaccagcgtcctcttaaaagatgccttaatccattccttgaggacagaccttagttgaaatgatagcagaatgtgcttctctctggcagctggccttctgcttctgagttgcacattaatcagattagcctgtattctcttcagtgaattttgataatggcttccagactctttggcgttggagacgcctgttaggatcttcaagtcccatcatagaaaattgaaacacagagttgttctgctgatagttttggggatacgtccatctttttaagggattgctttcatctaattctggcaggacctcaccaaaagatccagcctcatacctacatcagacaaaatatcgccgttgttccttctgtactaaagtattgtgttttgctttggaaacacccactcactttgcaatagccgtgcaagatgaatgcagattacactgatcttatgtgttacaaaattggagaaagtatttaataaaacctgttaatttttatactgacaataaaaatgtttctacagatattaatgttaacaagacaaaataaatgtcacgcaacttatttttttaataaaaaaaaaaaaaaa

SEQ ID NO: 154 - protein, 2948 aa, Homo sapiens
MGNENSTSDNQRTLSAQTPRSAQPPGNSQNIKRKQQDTPGSPDHRDASSIGSVGLGGFCTASESSASLDPCLVSPEVTEPRKDPQGARGPEGSLLPSPPPSQEREHPSSSMPFAECPPEGCLASPAAAPEDGPQTQSPRREPAPNAPGDIAAAFPAERDSSTPYQEIAAVPSAGRERQPKEEGQKSSFSFSSGIDQSPGMSPVPLREPMKAPLCGEGDQPGGFESQEKEAAGGFPPAESRQGVASVQVTPEAPAAAQQGTESSAVLEKSPLKPMAPIPQDPAPRASDRERGQGEAPPQYLTDDLEFLRACHLPRSNSGAAPEAEVNAASQESCQQPVGAYLPHAELPWGLPSPALVPEAGGSGKEALDTIDVQGHPQTGMRGTKPNQVVCVAAGGQPEGGLPVSPEPSLLTPTEEAHPASSLASFPAAQIPIAVEEPGSSSRESVSKAGMPVSADAAKEVVDAGLVGLERQVSDLGSKGEHPEGDPGEVPAPSPQERGEHLNTEQSHEVQPGVPPPPLPKEQSHEVQPGAPPPPLPKAPSESARGPPGPTDGAKVHEDSTSPAVAKEGSRSPGDSPGGKEEAPEPPDGGDPGNLQGEDSQAFSSKRDPEVGKDELSKPSSDAESRDHPSSHSAQPPRKGGAGHTDGPHSQTAEADASGLPHKLGEEDPVLPPVPDGAGEPTVPEGAIWEGSGLQPKCPDTLQSREGLGRMESFLTLESEKSDFPPTPVAEVAPKAQEGESTLEIRKMGSCDGEGLLTSPDQPRGPACDASRQEFHAGVPHPPQGENLAADLGLTALILDQDQQGIPSCPGEGWIRGAASEWPLLSSEKHLQPSQAQPETSIFDVLKEQAQPPENGKETSPSHPGFKDQGADSSQIHVPVEPQEDNNLPTHGGQEQALGSELQSQLPKGTLSDTPTSSPTDMVWESSLTEESELSAPTRQKLPALGEKRPEGACGDGQSSRVSPPAADVLKDFSLAGNFSRKETCCTGQGPNKSQQALADALEEGSQHEEACQRHPGASEAADGCSPLWGLSKREMASGNTGEAPPCQPDSVALLDAVPCLPALAPASPGVTPTQDAPETEACDETQEGRQQPVPAPQQKMECWATSDAESPKLLASFPSAGEQGGEAGAAETGGSAGAGDPGKQQAPEKPGEATLSCGLLQTEHCLTSGEEASTSALRESCQAEHPMASCQDALLPARELGGIPRSTMDFSTHQAVPDPKELLLSGPPEVAAPDTPYLHVDSAAQRGAEDSGVKAVSSADPRAPGESPCPVGEPPLALENAASLKLFAGSLAPLLQPGAAGGEIPAVQASSGSPKARTTEGPVDSMPCLDRMPLLAKGKQATGEEKAATAPGAGAKASGEGMAGDAAGETEGSMERMGEPSQDPKQGTSGGVDTSSEQIATLTGFPDFREHIAKIFEKPVLGALATPGEKAGAGRSAVGKDLTRPLGPEKLLDGPPGVDVTLLPAPPARLQVEKKQQLAGEAEISHLALQDPASDKLLGPAGLTWERNLPGAGVGKEMAGVPPTLREDERPEGPGAAWPGLEGQAYSQLERSRQELASGLPSPAATQELPVERAAAFQVAPHSHGEEAVAQDRIPSGKQHQETSACDSPHGEDGPGDFAHTGVPGHVPRSTCAPSPQREVLTVPEANSEPWTLDTLGGERRPGVTAGILEMRNALGNQSTPAPPTGEVADTPLEPGKVAGAAGEAEGDITLSTAETQACASGDLPEAGTTRTFSVVAGDLVLPGSCQDPACSDKAPGMEGTAALHGDSPARPQQAKEQPGPERPIPAGDGKVCVSSPPEPDETHDPKLQHLAPEELHTDRESPRPGPSMLPSVPKKDAPRVMDKVTSDETRGAEGTESSPVADDIIQPAAPADLESPTLAASSYHGDVVGQVSTDLIAQSISPAAAHAGLPPSAAEHIVSPSAPAGDRVEASTPSCPDPAKDLSRSSDSEEAFETPESTTPVKAPPAPPPPPPEVIPEPEVSTQPPPEEPGCGSETVPVPDGPRSDSVEGSPFRPPSHSFSAVFDEDKPIASSGTYNLDFDNIELVDTFQTLEPRASDAKNQEGKVNTRRKSTDSVPISKSTLSRSLSLQASDFDGASSSGNPEAVALAPDAYSTGSSSASSTLKRTKKPRPPSLKKKQTTKKPTETPPVKETQQEPDEESLVPSGENLASETKTESAKTEGPSPALLEETPLEPAVGPKAACPLDSESAEGVVPPASGGGRVQNSPPVGRKTLPLTTAPEAGEVTPSDSGGQEDSPAKGLSVRLEFDYSEDKSSWDNQQENPPPTKKIGKKPVAKMPLRRPKMKKTPEKLDNTPASPPRSPAEPNDIPIAKGTYTFDIDKWDDPNFNPFSSTSKMQESPKLPQQSYNFDPDTCDESVDPFKTSSKTPSSPSKSPASFEIPASAMEANGVDGDGLNKPAKKKKTPLKTDTFRVKKSPKRSPLSDPPSQDPTPAATPETPPVISAVVHATDEEKLAVTNQKWTCMTVDLEADKQDYPQPSDLSTFVNETKFSSPTEELDYRNSYEIEYMEKIGSSLPQDDDAPKKQALYLMFDTSQESPVKSSPVRMSESPTPCSGSSFEETEALVNTAAKNQHPVPRGLAPNQESHLQVPEKSSQKELEAMGLGTPSEAIEITAPEGSFASADALLSRLAHPVSLCGALDYLEPDLAEKNPPLFAQKLQEELEFAIMRIEALKLARQIALASRSHQDAKREAAHPTDVSISKTALYSRIGTAEVEKPAGLLFQQPDLDSALQIARAEIITKEREVSEWKDKYEESRREVMEMRKIVAEYEKTIAQMIEDEQREKSVSHQTVQQLVLEKEQALADLNSVEKSLADLFRRYEKMKEVLEGFRKNEEVLKRCAQEYLSRVKKEEQRYQALKVHAEEKLDRANAEIAQVRGKAQQEQAAHQASLRKEQLRVDALERTLEQKNKEIEELTKICDELIAKMGKS

SEQ ID NO: 155 - DNA, 9706 bp, Homo sapiens
  source 1..9706: mol_type = unassigned DNA
gcctgctccaagggaaggatcaggagagaagaaacgcaaatcccagaaccgtgccaacatataaaaccccacattaagggttgtacagtgcactgggatttctcaagtcacccgcttggtcctcttccaagtatactttacttcctttcattcctctctaaaacttttttaaaaactttcactcctgctctaaaagttatcttggtttcttactctaccttatgccccttgggcgaattttttcctctgaggagggaagaatagagttgctgctgcagacacatcagattccctactggtaacagctggagtgcgtcacctctgacaaaattctggggacgctgggaacactgaatcaacatgggcaatgagaacagcacctcggacaaccagaggactttatcagctcagactccaaggtccgcgcagccacccgggaacagtcagaatataaaaaggaagcagcaggacacgcccggaagccctgaccacagagacgcgtccagcattggcagcgttgggcttggaggcttctgcaccgcttctgagagttctgccagcctggatccatgccttgtgtccccagaggtgactgagccaaggaaggacccacagggagccagggggccagaaggttctttgctgcccagcccaccaccgtcccaggagcgagagcacccctcgtcctccatgccctttgccgagtgtcccccggaaggttgcttggcaagtccagcagcggcacctgaagatggtcctcagactcagtctcccaggagggaacctgccccaaatgccccaggagacatcgcggcggcatttcccgctgagagggacagctctactccataccaagagattgctgccgtccccagtgctggaagagagagacagccgaaggaagaaggacagaagtcctccttctccttctccagtggcatcgaccagtcacctggaatgtcgccagtacccctcagagagccaatgaaggcaccgctgtgtggagagggggaccagcctggtggttttgagtcccaagagaaagaggctgcaggtggctttccccctgcagagtccaggcagggggtggcttctgtgcaagtgacccctgaggcccctgctgcagcccagcagggcacagaaagctcagcggtcttggagaagtcccccctaaaacccatggccccgatcccacaagatccagccccaagagcctcagacagagaaagaggccaaggggaggcgccgcctcagtatttaacagatgacttggaattcctcagggcctgccatctccctaggagcaattcaggggctgccccagaagcagaagtgaatgccgcttcccaggagagctgccagcagccagtgggagcatatctgccgcacgcagagctgccctggggcttgccaagtcctgccctggtgccagaggctgggggctctgggaaggaggctctggacaccattgatgttcagggtcacccacagacagggatgcgaggaaccaagcccaatcaagttgtctgtgtggcagcaggcggccagcccgaagggggtttgcctgtgagccctgaaccttccctgctcactccgactgaggaagcacatccagcttcaagcctcgcttcattcccagctgctcagattcctattgctgtagaagaacctggatcatcatccagggaatcagtttccaaggctgggatgccagtttctgcagatgcagccaaagaggtggtggatgcagggttggtgggactggagaggcaggtgtcagatcttggaagcaagggagagcatccagaaggggaccctggagaggttcctgccccatcaccccaggagaggggagagcacttgaacacggagcaaagccatgaggtccaaccaggagtaccaccccctcctcttcccaaggagcaaagccatgaggtccaaccaggagcaccaccccctcctcttcccaaggcaccaagtgaaagtgccagagggccaccggggccaacggatggagccaaggtccatgaagattccacaagcccagccgtggctaaagaaggaagcagatcacctggtgacagccctggaggaaaggaggaagccccagagccacctgatggtggagacccagggaacctgcaaggagaggactctcaggctttcagcagcaagcgtgatccagaagtaggcaaagatgagctttcaaagccaagcagtgatgcagagagcagagaccatcccagctcacactcagcacagccacccagaaaggggggtgctgggcacacggacgggccccactctcagacagcagaggctgatgcatctggcctaccacacaagctgggtgaggaggaccccgtcctgccccctgtgccagatggagctggtgagcccactgttcccgaaggagccatctgggaggggtcaggattgcagcccaaatgtcctgacacccttcagagcagggaaggattgggaagaatggagtctttcctgactttagaatcagagaaatcagattttccaccaactcctgttgcagaggttgcacccaaagcccaggaaggtgagagcacattggaaataaggaagatgggcagctgtgatggggagggcttgctgacgtccccagatcaaccccgcgggccggcgtgtgatgcgtcgagacaggaatttcatgctggggtgccacatcccccccagggggagaacttggcagcagacctggggctcacggcactcatcctggaccaagatcagcagggaatcccatcctgcccaggggaaggctggataagaggagctgcatccgagtggcccctactatcttctgagaagcatctccagccatcccaggcacaaccagagacatccatctttgacgtgctcaaggagcaggcccagccacctgaaaatgggaaagagacttctccaagccatccaggttttaaggaccagggagcagattcttcccaaatccatgtacctgtggaacctcaggaagataacaacttgcccactcatggaggacaggagcaggctttgggatcagaacttcaaagtcagctccccaaaggcaccctgtctgatactccaacttcatctcccactgacatggtttgggagagttctctgacagaagagtcagaattgtcagcaccaacgagacagaagttgcctgcactaggggagaagcggccagagggagcatgcggtgatggtcagtcctcgagggtctcgcctccagcagcagatgtcttaaaagacttttctcttgcagggaacttcagcagaaaggaaacttgctgcactgggcaggggccaaacaagtctcaacaggcattggctgatgccttggaagaaggcagccagcatgaagaagcatgtcaaaggcatccaggagcttctgaagcagctgatggttgttccccactctggggcttgagtaagagggagatggcaagtggaaacacaggggaggccccaccttgtcagcctgactcagtagctctcctggatgcagttccctgcctgccagccctggcgcccgccagccccggagtcacacccacccaggatgccccagagacagaggcatgtgatgaaacccaggaaggcaggcagcaaccagtgccggccccgcagcagaaaatggagtgctgggccacttcggatgcagagtccccaaagcttcttgcaagtttcccatcagctggggagcaaggtggtgaagccggggctgctgagactggtggcagcgctggtgcaggagacccaggaaagcagcaggctccggagaaacctggagaagctactttgagttgtggcctccttcagactgagcactgccttacctccggggaggaagcttctacctctgccctacgtgagtcctgccaagctgagcaccccatggccagctgccaggatgccttgctgccagccagagagctgggtgggattcccaggagcaccatggatttttctacacaccaggctgtcccagacccaaaggagctcctgctgtctgggccaccagaagtggctgctcctgacaccccttacctgcatgtcgacagtgctgcccagagaggagcagaagacagtggagtgaaagctgtttcctctgcagaccccagagctcctggcgaaagcccctgtcctgtaggggagcccccacttgccttggaaaatgctgcctccttgaagctgtttgctggctccctcgcccccctgttgcaaccaggagctgcaggtggggaaatccctgcagtgcaagccagcagtggtagtcccaaagccagaaccactgagggaccagtggactccatgccatgcctggaccggatgccacttctggccaagggcaagcaggcaacaggggaagagaaagcagcaacagctccaggtgcaggtgccaaggccagtggggagggcatggcaggtgatgcagcaggagagacagagggcagcatggagaggatgggagagccttcccaggacccaaagcagggcacatcaggtggtgtggacacaagctctgagcaaatcgccaccctcactggcttcccagacttcagggagcacatcgccaagatcttcgagaagcctgtgctcggagccctggccacacctggagaaaaggcaggagctgggaggagtgcagtgggtaaagacctcaccaggccattgggcccagagaagcttctagatgggcctccaggagtggatgtcacccttctccctgcacctcctgctcgactccaggtggagaagaagcaacagttggctggagaggctgagatttcccatctggctctgcaagatccagcttcagacaagcttctgggtccagcagggctgacctgggagcggaacttgccaggtgccggtgtggggaaggagatggcaggtgtcccacccacactgagggaagacgagaggccagaggggcctggggcagcctggccaggcctggaaggccaggcttactcacagctggagaggagcaggcaggaattagcttcaggtcttccttcaccagcagctactcaggagctccctgtggagagagctgctgccttccaggtggctccccatagccatggagaagaggccgtggcccaagacagaattccttctggaaagcagcaccaggaaacatctgcctgcgacagtccacatggagaagatggtcccggggactttgctcacacaggggttccaggacatgtgccaaggtccacgtgtgccccttctcctcagagggaggttttgactgtgcctgaggccaacagtgagccctggacccttgacacgcttgggggtgaaaggagacccggagtcactgctggcatcttggaaatgcgaaatgccctgggcaaccagagcacccctgcaccaccaactggagaagtggcagacactcccctggagcctggcaaggtggcaggcgctgctggggaagcagagggtgacatcaccctgagcacagctgagacacaggcatgtgcgtccggtgatctgcctgaagcaggtactacgaggacattctccgttgtggcaggtgacttggtgctgccaggaagctgtcaggacccagcctgctctgacaaggctccggggatggagggtacagctgcccttcatggggacagcccagccaggccccagcaggctaaggagcagccagggcctgagcgccccattccagctggggatgggaaggtgtgcgtctcctcacctccagagcctgacgaaactcacgacccgaagctgcaacatttggctccagaagagctccacactgacagagagagccccaggcctggcccatccatgttaccttcggttcctaagaaggatgctccaagagtcatggataaagtcacttcagatgagaccagaggtgcggaaggaacagaaagttcacctgtggcagatgatatcatccagcccgctgcccccgcagacctggaaagcccaaccttagctgcctcttcctaccacggtgatgttgttggccaggtctctacggatctgatagcccagagcatctccccagctgctgcccatgcgggtcttcctccctcggctgcagaacacatagtttcgccatctgccccagctggtgacagagtagaagcttccactccctcctgcccagatccggccaaggacctcagcaggagttccgattctgaagaggcatttgagaccccggagtcaacgacccctgtcaaagctccgccagctccacccccaccaccccccgaagtcatcccagaacccgaggtcagcacacagccacccccggaagaaccaggatgtggttctgagacagtccctgtccctgatggcccacggagcgactcggtggaaggaagtcccttccgtcccccgtcacactccttctctgccgtcttcgatgaagacaagccgatagccagcagtgggacttacaacttggactttgacaacattgagcttgtggatacctttcagaccttggagcctcgtgcctcagacgctaagaatcaggagggcaaagtgaacacacggaggaagtccacggattccgtccccatctctaagtctacactgtcccggtcgctcagcctgcaagccagtgactttgatggtgcttcttcctcaggcaatcccgaggccgtggcccttgccccagatgcatatagcacgggttccagcagtgcttctagtacccttaagcgaactaaaaaaccgaggccgccttccttaaaaaagaaacagaccaccaagaaacccacagagacccccccagtgaaggagacgcaacaggagccagatgaagagagccttgtccccagtggggagaatctagcatctgagacgaaaacggaatctgccaagacggaaggtcctagcccagccttattggaggagacgccccttgagcccgctgtggggcccaaagctgcctgccctctggactcagagagtgcagaaggggttgtccccccggcttctggaggtggcagagtgcagaactcaccccctgtcgggaggaaaacgctgcctcttaccacggccccggaggcaggggaggtaaccccatcggatagcggggggcaagaggactctccagccaaagggctctccgtaaggctggagtttgactattctgaggacaagagtagttgggacaaccagcaggaaaacccccctcctaccaaaaagataggcaaaaagccagttgccaaaatgcccctgaggaggccaaagatgaaaaagacacccgagaaacttgacaacactcctgcctcacctcccagatcccctgctgaacccaatgacatccccattgctaaaggtacttacacctttgatattgacaagtgggatgaccccaattttaaccctttttcttccacctcaaaaatgcaggagtctcccaaactgccccaacaatcatacaactttgacccagacacctgtgatgagtccgttgacccctttaagacatcctctaagacccccagctcaccttctaaatccccagcctcctttgagatcccagccagtgctatggaagccaatggagtggacggggatgggctaaacaagcccgccaagaagaagaagacgcccctaaagactgacacatttagggtgaaaaagtcgccaaaacggtctcctctctctgatccaccttcccaggaccccaccccagctgctacaccagaaacaccaccagtgatctctgcggtggtccacgccacagatgaggaaaagctggcggtcaccaaccagaagtggacgtgcatgacagtggacctagaggctgacaaacaggactacccgcagccctcggacctgtccacctttgtaaacgagaccaaattcagttcacccactgaggagttggattacagaaactcctatgaaattgaatatatggagaaaattggctcctccttacctcaggacgacgatgccccgaagaagcaggccttgtaccttatgtttgacacttctcaggagagccctgtcaagtcatctcccgtccgcatgtcagagtccccgacgccgtgttcagggtcaagttttgaagagactgaagcccttgtgaacactgctgcgaaaaaccagcatcctgtcccacgaggactggcccctaaccaagagtcacacttgcaggtgccagagaaatcctcccagaaggagctggaggccatgggcttgggcaccccttcagaagcgattgaaattacagctcccgagggctcctttgcctctgctgacgccctcctcagcaggctagctcaccccgtctctctctgtggtgcacttgactatctggagcccgacttagcagaaaagaaccccccactattcgctcagaaactccaggaggagttagagtttgccatcatgcggatagaagccctgaagctggccaggcagattgctttggcttcccgcagccaccaggatgccaagagagaggctgctcacccaacagacgtctccatctccaaaacagccttgtactcccgcatcgggaccgctgaggtggagaaacctgcaggccttctgttccagcagcccgacctggactctgccctccagatcgccagagcagagatcataaccaaggagagagaggtctcagaatggaaagataaatatgaagaaagcaggcgggaagtgatggaaatgaggaaaatagtggccgagtatgagaagaccatcgctcagatgatagaggacgaacagagagagaagtcagtctcccaccagacggtgcagcagctggttctggagaaggagcaagccctggccgacctgaactccgtggagaagtctctggccgacctcttcagaagatatgagaagatgaaggaggtcctagaaggcttccgcaagaatgaagaggtgttgaagagatgtgcgcaggagtacctgtcccgggtgaagaaggaggagcagaggtaccaggccctgaaggtgcacgcggaggagaaactggacagggccaatgctgagattgctcaggttcgaggcaaggcccagcaggagcaagccgcccaccaggccagcctgcggaaggagcagctgcgagtggacgccctggaaaggacgctggagcagaagaataaagaaatagaagaactcaccaagatttgtgacgaactgattgccaaaatggggaaaagctaactctgaaccgaatgttttggacttaactgttgcgtgcaatatgaccgtcggcacactgctgttcctccagttccatggacaggttctgttttcactttttcgtatgcactactgtatttcctttctaaataaaattgatttgattgtatgcagtactaaggagactatcagaatttcttgctattggtttgcattttcctagtataattcatagcaagttgacctcagagttcctgtatcagggagattgtctgattctctaataaaagacacattgctgaccttggccttgccctttgtacacaagttcccagggtgagcagcttttggatttaatatgaacatgtacagcgtgcatagggactcttgccttaaggagtgtaaacttgatctgcatttgctgatttgtttttaaaaaaacaagaaatgcatgtttcaaataaaattctctattgtaaataaaattttttctttggatcttggcaaaaaaaaaaaaaaa

SEQ ID NO: 156 - protein, 13 aa, synthetic construct
  REGION 1..13: note = Description of Artificial Sequence: Synthetic peptide
AQAEALALQASLR

SEQ ID NO: 157 - protein, 6 aa, synthetic construct
  REGION 1..6: note = Description of Artificial Sequence: Synthetic peptide
FDPLLR

SEQ ID NO: 158 - protein, 1048 aa, synthetic construct; identical to SEQ ID NO: 79
  REGION 1..1048: note = Description of Artificial Sequence: Synthetic polypeptide

SEQ ID NO: 159 - protein, 1027 aa, synthetic construct
  REGION 1..1027: note = Description of Artificial Sequence: Synthetic polypeptide
MGAPACALALCVAVAIVAGASSESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAGILSYGVGFFLFILVVAAVTLCRLRSPPKKGLGSPTVHKISRFPLKRQVSLESNASMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREFLRARRPPGLDYSFDTCKPPEEQLTFKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAAPSQRPTFKQLVEDLDRVLTVTSTDVSAGSGLVPPAYAPPPAVPGHPSGRPREAKLVEFDFLGALDIPVPGPPPGVPAPGGPPLSTGPIVDLLQYSQKDLDAVVKATQEENRELRSRCEELHGKNLELGKIMDRFEEVVYQAMEEVQKQKELSKAEIQKVLKEKDQLTTDLNSMEKSFSDLFKRFEKQKEVIEGYRKNEESLKKCVEDYLARITQEGQRYQALKAHAEEKLQLANEEIAQVRSKAQAEALALQASLRKEQMRIQSLEKTVEQKTKENEELTRICDDLISKMEKI

SEQ ID NO: 160 - protein, 984 aa, synthetic construct
  REGION 1..984: note = Description of Artificial Sequence: Synthetic polypeptide
MGAPACALALCVAVAIVAGASSESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAGILSYGVGFFLFILVVAAVTLCRLRSPPKKGLGSPTVHKISRFPLKRQVSLESNASMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREFLRARRPPGLDYSFDTCKPPEEQLTFKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAAPSQRPTFKQLVEDLDRVLTVTSTDVPGPPPGVPAPGGPPLSTGPIVDLLQYSQKDLDAVVKATQEENRELRSRCEELHGKNLELGKIMDRFEEVVYQAMEEVQKQKELSKAEIQKVLKEKDQLTTDLNSMEKSFSDLFKRFEKQKEVIEGYRKNEESLKKCVEDYLARITQEGQRYQALKAHAEEKLQLANEEIAQVRSKAQAEALALQASLRKEQMRIQSLEKTVEQKTKENEELTRICDDLISKMEKI

SEQ ID NO: 161 - protein, 949 aa, synthetic construct
  REGION 1..949: note = Description of Artificial Sequence: Synthetic polypeptide
MGAPACALALCVAVAIVAGASSESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAGILSYGVGFFLFILVVAAVTLCRLRSPPKKGLGSPTVHKISRFPLKRQVSLESNASMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREFLRARRPPGLDYSFDTCKPPEEQLTFKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAAPSQRPTFKQLVEDLDRVLTVTSTDVKATQEENRELRSRCEELHGKNLELGKIMDRFEEVVYQAMEEVQKQKELSKAEIQKVLKEKDQLTTDLNSMEKSFSDLFKRFEKQKEVIEGYRKNEESLKKCVEDYLARITQEGQRYQALKAHAEEKLQLANEEIAQVRSKAQAEALALQASLRKEQMRIQSLEKTVEQKTKENEELTRICDDLISKMEKI

SEQ ID NO: 162 - DNA, 20 bp, synthetic construct
  misc_feature 1..20: note = Description of Artificial Sequence: Synthetic primer
  source 1..20: mol_type = other DNA
gtaacctgcgggagtttctg

SEQ ID NO: 163 - DNA, 20 bp, synthetic construct
  misc_feature 1..20: note = Description of Artificial Sequence: Synthetic primer
  source 1..20: mol_type = other DNA
acaccaggtccttgaaggtg

SEQ ID NO: 164 - DNA, 20 bp, synthetic construct
  misc_feature 1..20: note = Description of Artificial Sequence: Synthetic primer
  source 1..20: mol_type = other DNA
cctgagggacagtcctggta

SEQ ID NO: 165 - DNA, 20 bp, synthetic construct
  misc_feature 1..20: note = Description of Artificial Sequence: Synthetic primer
  source 1..20: mol_type = other DNA
agtgctcccaagaaatcgaa

SEQ ID NO: 166 - DNA, 23 bp, synthetic construct
  misc_feature 1..23: note = Description of Artificial Sequence: Synthetic primer
  source 1..23: mol_type = other DNA
cgtgaagatgctgaaagacgatg

SEQ ID NO: 167 - DNA, 21 bp, synthetic construct
  misc_feature 1..21: note = Description of Artificial Sequence: Synthetic primer
  source 1..21: mol_type = other DNA
aaacgcttgaagaggtcggag

SEQ ID NO: 168 - DNA, 20 bp, synthetic construct
  misc_feature 1..20: note = Description of Artificial Sequence: Synthetic primer
  source 1..20: mol_type = other DNA
atgctagcaggggtctctga

SEQ ID NO: 169 - DNA, 20 bp, synthetic construct
  misc_feature 1..20: note = Description of Artificial Sequence: Synthetic primer
  source 1..20: mol_type = other DNA
cccttccagaacacctttca

SEQ ID NO: 170 - DNA, 18 bp, synthetic construct
  misc_feature 1..18: note = Description of Artificial Sequence: Synthetic primer
  source 1..18: mol_type = other DNA
atgatcatgcgggagtgc

SEQ ID NO: 171 - DNA, 20 bp, synthetic construct
  misc_feature 1..20: note = Description of Artificial Sequence: Synthetic primer
  source 1..20: mol_type = other DNA
gggggtcgaacttgaggtat

SEQ ID NO: 172 - DNA, 20 bp, synthetic construct
  misc_feature 1..20: note = Description of Artificial Sequence: Synthetic primer
  source 1..20: mol_type = other DNA
cgcaggctttttgtagtgag

SEQ ID NO: 173 - DNA, 18 bp, synthetic construct
  misc_feature 1..18: note = Description of Artificial Sequence: Synthetic primer
  source 1..18: mol_type = other DNA
tgtaggcgcgaaaggaag

SEQ ID NO: 174 - DNA, 20 bp, synthetic construct
  misc_feature 1..20: note = Description of Artificial Sequence: Synthetic primer
  source 1..20: mol_type = other DNA
gaactcatccggacccctat

SEQ ID NO: 175 - DNA, 20 bp, synthetic construct
  misc_feature 1..20: note = Description of Artificial Sequence: Synthetic primer
  source 1..20: mol_type = other DNA
gctttccccattgcacttta

SEQ ID NO: 176 - DNA, 20 bp, synthetic construct
  misc_feature 1..20: note = Description of Artificial Sequence: Synthetic primer
  source 1..20: mol_type = other DNA
gaggagagaagcacgtggag

SEQ ID NO: 177 - DNA, 20 bp, synthetic construct
  misc_feature 1..20: note = Description of Artificial Sequence: Synthetic primer
  source 1..20: mol_type = other DNA
ggcagacgtgtgaggtgtaa

SEQ ID NO: 178 - DNA, 20 bp, synthetic construct
  misc_feature 1..20: note = Description of Artificial Sequence: Synthetic primer
  source 1..20: mol_type = other DNA
gtgatcagcagctggactgt

SEQ ID NO: 179 - DNA, 18 bp, synthetic construct
  misc_feature 1..18: note = Description of Artificial Sequence: Synthetic primer
  source 1..18: mol_type = other DNA
gagcctgggcatggatct

SEQ ID NO: 180 - DNA, 20 bp, synthetic construct
  misc_feature 1..20: note = Description of Artificial Sequence: Synthetic primer
  source 1..20: mol_type = other DNA
agaggtgaccaccaatcagc

SEQ ID NO: 181 - DNA, 20 bp, synthetic construct
  misc_feature 1..20: note = Description of Artificial Sequence: Synthetic primer
  source 1..20: mol_type = other DNA
cgtgtcccacacagagacag

SEQ ID NO: 182 - DNA, 21 bp, synthetic construct
  misc_feature 1..21: note = Description of Artificial Sequence: Synthetic oligonucleotide
  source 1..21: mol_type = other DNA
tgcgtcgtggagaacaagttt

SEQ ID NO: 183 - DNA, 21 bp, synthetic construct
  misc_feature 1..21: note = Description of Artificial Sequence: Synthetic oligonucleotide
  source 1..21: mol_type = other DNA
gttccactgcaaggtgtacag

SEQ ID NO: 184 - DNA, 21 bp, synthetic construct
  misc_feature 1..21: note = Description of Artificial Sequence: Synthetic oligonucleotide
  source 1..21: mol_type = other DNA
gcacaacctcgactactacaa

SEQ ID NO: 185 - protein, 822 aa, Homo sapiens [sequence omitted]

SEQ ID NO: 186 - DNA, 5917 bp, Homo sapiens
  source 1..5917: mol_type = unassigned DNA
agatgcaggggcgcaaacgccaaaggagaccaggctgtaggaagagaagggcagagcgccggacagctcggcccgctccccgtcctttggggccgcggctggggaactacaaggcccagcaggcagctgcagggggcggaggcggaggagggaccagcgcgggtgggagtgagagagcgagccctcgcgccccgccggcgcatagcgctcggagcgctcttgcggccacaggcgcggcgtcctcggcggcgggcggcagctagcgggagccgggacgccggtgcagccgcagcgcgcggaggaacccgggtgtgccgggagctgggcggccacgtccggacgggaccgagacccctcgtagcgcattgcggcgacctcgccttccccggccgcgagcgcgccgctgcttgaaaagccgcggaacccaaggacttttctccggtccgagctcggggcgccccgcagggcgcacggtacccgtgctgcagtcgggcacgccgcggcgccggggcctccgcagggcgatggagcccggtctgcaaggaaagtgaggcgccgccgctgcgttctggaggaggggggcacaaggtctggagaccccgggtggcggacgggagccctccccccgccccgcctccggggcaccagctccggctccattgttcccgcccgggctggaggcgccgagcaccgagcgccgccgggagtcgagcgccggccgcggagctcttgcgaccccgccaggacccgaacagagcccgggggcggcgggccggagccggggacgcgggcacacgcccgctcgcacaagccacggcggactctcccgaggcggaacctccacgccgagcgagggtcagtttgaaaaggaggatcgagctcactgtggagtatccatggagatgtggagccttgtcaccaacctctaactgcagaactgggatgtggagctggaagtgcctcctcttctgggctgtgctggtcacagccacactctgcaccgctaggccgtccccgaccttgcctgaacaagcccagccctggggagcccctgtggaagtggagtccttcctggtccaccccggtgacctgctgcagcttcgctgtcggctgcgggacgatgtgcagagcatcaactggctgcgggacggggtgcagctggcggaaagcaaccgcacccgcatcacaggggaggaggtggaggtgcaggactccgtgcccgcagactccggcctctatgcttgcgtaaccagcagcccctcgggcagtgacaccacctacttctccgtcaatgtttcagatgctctcccctcctcggaggatgatgatgatgatgatgactcctcttcagaggagaaagaaacagataacaccaaaccaaaccgtatgcccgtagctccatattggacatccccagaaaagatggaaaagaaattgcatgcagtgccggctgccaagacagtgaagttcaaatgcccttccagtgggaccccaaaccccacactgcgctggttgaaaaatggcaaagaattcaaacctgaccacagaattggaggctacaaggtccgttatgccacctggagcatcataatggactctgtggtgccctctgacaagggcaactacacctgcattgtggagaatgagtacggcagcatcaaccacacataccagctggatgtcgtggagcggtcccctcaccggcccatcctgcaagcagggttgcccgccaacaaaacagtggccctgggtagcaacgtggagttcatgtgtaaggtgtacagtgacccgcagccgcacatccagtggctaaagcacatcgaggtgaatgggagcaagattggcccagacaacctgccttatgtccagatcttgaagactgctggagttaataccaccgacaaagagatggaggtgcttcacttaagaaatgtctcctttgaggacgcaggggagtatacgtgcttggcgggtaactctatcggactctcccatcactctgcatggttgaccgttctggaagccctggaagagaggccggcagtgatgacctcgcccctgtacctggagatcatcatctattgcacaggggccttcctcatctcctgcatggtggggtcggtcatcgtctacaagatgaagagtggtaccaagaagagtgacttccacagccagatggctgtgcacaagctggccaagagcatccctctgcgcagacaggtaacagtgtctgctgactccagtgcatccatgaactctggggttcttctggttcggccatcacggctctcctccagtgggactcccatgctagcaggggtctctgagtatgagcttcccgaagaccctcgctgggagctgcctcgggacagactggtcttaggcaaacccctgggagagggctgctttgggcaggtggtgttggcagaggctatcgggctggacaaggacaaacccaaccgtgtgaccaaagtggctgtgaagatgttgaagtcggacgcaacagagaaagacttgtcagacctgatctcagaaatggagatgatgaagatgatcgggaagcataagaatatcatcaacctgctgggggcctgcacgcaggatggtcccttgtatgtcatcgtggagtatgcctccaagggcaacctgcgggagtacctgcaggcccggaggcccccagggctggaatactgctacaaccccagccacaacccagaggagcagctctcctccaaggacctggtgtcctgcgcctaccaggtggcccgaggcatggagtatctggcctccaagaagtgcatacaccgagacctggcagccaggaatgtcctggtgacagaggacaatgtgatgaagatagcagactttggcctcgcacgggacattcaccacatcgactactataaaaagacaaccaacggccgactgcctgtgaagtggatggcacccgaggcattatttgaccggatctacacccaccagagtgatgtgtggtctttcggggtgctcctgtgggagatcttcactctgggcggctccccataccccggtgtgcctgtggaggaacttttcaagctgctgaaggagggtcaccgcatggacaagcccagtaactgcaccaacgagctgtacatgatgatgcgggactgctggcatgcagtgccctcacagagacccaccttcaagcagctggtggaagacctggaccgcatcgtggccttgacctccaaccaggagtacctggacctgtccatgcccctggaccagtactcccccagctttcccgacacccggagctctacgtgctcctcaggggaggattccgtcttctctcatgagccgctgcccgaggagccctgcctgccccgacacccagcccagcttgccaatggcggactcaaacgccgctgactgccacccacacgccctccccagactccaccgtcagctgtaaccctcacccacagcccctgctgggcccaccacctgtccgtccctgtcccctttcctgctggcaggagccggctgcctaccaggggccttcctgtgtggcctgccttcaccccactcagctcacctctccctccacctcctctccacctgctggtgagaggtgcaaagaggcagatctttgctgccagccacttcatcccctcccagatgttggaccaacacccctccctgccaccaggcactgcctggagggcagggagtgggagccaatgaacaggcatgcaagtgagagcttcctgagctttctcctgtcggtttggtctgttttgccttcacccataagcccctcgcactctggtggcaggtgccttgtcctcagggctacagcagtagggaggtcagtgcttcgtgcctcgattgaaggtgacctctgccccagataggtggtgccagtggcttattaattccgatactagtttgctttgctgaccaaatgcctggtaccagaggatggtgaggcgaaggccaggttgggggcagtgttgtggccctggggcccagccccaaactgggggctctgtatatagctatgaagaaaacacaaagtgtataaatctgagtatatatttacatgtctttttaaaagggtcgttaccagagatttacccatcgggtaagatgctcctggtggctgggaggcatcagttgctatatattaaaaacaaaaaagaaaaaaaaggaaaatgtttttaaaaaggtcatatattttttgctacttttgctgttttatttttttaaattatgttctaaacctattttcagtttaggtccctcaataaaaattgctgctgcttcatttatctatgggctgtatgaaaagggtgggaatgtccactggaaagaagggacacccacgggccctggggctaggtctgtcccgagggcaccgcatgctcccggcgcaggttccttgtaacctcttcttcctaggtcctgcacccagacctcacgacgcacctcctgcctctccgctgcttttggaaagtcagaaaaagaagatgtctgcttcgagggcaggaaccccatccatgcagtagaggcgctgggcagagagtcaaggcccagcagccatcgaccatggatggtttcctccaaggaaaccggtggggttgggctggggagggggcacctacctaggaatagccacggggtagagctacagtgattaagaggaaagcaagggcgcggttgctcacgcctgtaatcccagcactttgggacaccgaggtgggcagatcacttcaggtcaggagtttgagaccagcctggccaacttagtgaaaccccatctctactaaaaatgcaaaaattatccaggcatggtggcacacgcctgtaatcccagctccacaggaggctgaggcagaatcccttgaagctgggaggcggaggttgcagtgagccgagattgcgccattgcactccagcctgggcaacagagaaaacaaaaaggaaaacaaatgatgaaggtctgcagaaactgaaacccagacatgtgtctgccccctctatgtgggcatggttttgccagtgcttctaagtgcaggagaacatgtcacctgaggctagttttgcattcaggtccctggcttcgtttcttgttggtatgcctccccagatcgtccttcctgtatccatgtgaccagactgtatttgttgggactgtcgcagatcttggcttcttacagttcttcctgtccaaactccatcctgtccctcaggaacggggggaaaattctccgaatgtttttggttttttggctgcttggaatttacttctgccacctgctggtcatcactgtcctcactaagtggattctggctcccccgtacctcatggctcaaactaccactcctcagtcgctatattaaagcttatattttgctggattactgctaaatacaaaagaaagttcaatatgttttcatttctgtagggaaaatgggattgctgctttaaatttctgagctagggattttttggcagctgcagtgttggcgactattgtaaaattctctttgtttctctctgtaaatagcacctgctaacattacaatttgtatttatgtttaaagaaggcatcatttggtgaacagaactaggaaatgaatttttagctcttaaaagcatttgctttgagaccgcacaggagtgtctttccttgtaaaacagtgatgataatttctgccttggccctaccttgaagcaatgttgtgtgaagggatgaagaatctaaaagtcttcataagtccttgggagaggtgctagaaaaatataaggcactatcataattacagtgatgtccttgctgttactactcaaatcacccacaaatttccccaaagactgcgctagctgtcaaataaaagacagtgaaattgacctgaaaaaaaaaaaaaaaaa

SEQ ID NO: 187 - DNA, 76 bp, Homo sapiens
  source 1..76: mol_type = unassigned DNA
gtgctggcatgccgcgccctcccagaggcccaccttcaagcagctggtggaggacctggaccgtgtccttaccgtg

SEQ ID NO: 188 - DNA, 76 bp, Homo sapiens
  source 1..76: mol_type = unassigned DNA
atgcgggagtgctggcatgacgcgccctcccagaggcccaccttcaagcagctggtggaggacctggaccgtgtcc

SEQ ID NO: 189 - DNA, 76 bp, Homo sapiens
  source 1..76: mol_type = unassigned DNA
agctggtggaggacctggaccgtgtccttaccgtgacgtccaccgacgtgagtgctggctctggcctggtgccacc

SEQ ID NO: 190 - DNA, 76 bp, Homo sapiens
  source 1..76: mol_type = unassigned DNA
cccttaaaacaactcgttccctcagaccacacacaagacagttcaagagggactcaaggacttacaggaatgtcca

SEQ ID NO: 191 - DNA, 76 bp, Homo sapiens
  source 1..76: mol_type = unassigned DNA
aaccaaaggctcagacccccaggaatagaaaatataggcccttaaaacaactcgttccctcagaccacacacaaga

SEQ ID NO: 192 - DNA, 76 bp, Homo sapiens
  source 1..76: mol_type = unassigned DNA
tcaaggacttacaggaatgtccagtgctcccaagaaatcgaactccacaagcttggcttcccgcgcacgtcctgag

SEQ ID NO: 193 - DNA, 76 bp, Homo sapiens
  source 1..76: mol_type = unassigned DNA
ggacttacaggaatgtccagtgctcccaagaaatcgaactccacaagcttggcttcccgcggacgtcctgagggat

SEQ ID NO: 194 - DNA, 76 bp, Homo sapiens
  source 1..76: mol_type = unassigned DNA
cagaccacacacaagacagttcaagagggactcaaggacttacaggaatgtccagtgctcccaagaaatcgaactc

SEQ ID NO: 195 - DNA, 76 bp, Homo sapiens
  source 1..76: mol_type = unassigned DNA
cttaccgtgacgtccaccgacgtgagtgctggctctggcctggtgccacccgcctatgcccctcccctgcccttag

SEQ ID NO: 196 - DNA, 76 bp, Homo sapiens
  source 1..76: mol_type = unassigned DNA
aaacttgaggtataaggactgcttcctcaaggccgactccttaaactggggacaagagggcaagtgatcaggtctg

SEQ ID NO: 197 - DNA, 76 bp, Homo sapiens
  source 1..76: mol_type = unassigned DNA
aacttgaggtataaggactgcttcctcaaggccgactccttaaactggggacaagagggcaagtgatcaggtctga

SEQ ID NO: 198 - DNA, 76 bp, Homo sapiens
  source 1..76: mol_type = unassigned DNA
gcccgcaggtacatgatcatgcgggagtgctggcatgccgcgccctcccagaggcccaccttcaagcagctggtgg

SEQ ID NO: 199 - DNA, 76 bp, Homo sapiens
  source 1..76: mol_type = unassigned DNA
accgtgacgtccaccgacgtgagtgctggctctggcctggtgcgacccgccgatctctctcccctgtccttttcct

SEQ ID NO: 200 - DNA, 76 bp, Homo sapiens
  source 1..76: mol_type = unassigned DNA
tgggagggtgcggggggccgggggggggagtgtgcaggtgagctccctggcccttggccccctgccctctgggggg

SEQ ID NO: 201 - DNA, 76 bp, Homo sapiens
  source 1..76: mol_type = unassigned DNA
ctgggaatggtggtgtctcgggcagggttgtgggtgaccgggggtgggagggtgcgggggaccgggggggggaggg

SEQ ID NO: 202 - DNA, 76 bp, Homo sapiens
  source 1..76: mol_type = unassigned DNA
agcgccctgcccgcaggtacatgatcatgcgggagtgctggcatgccgcgccctcccagaggcccaccttcaagca

SEQ ID NO: 203 - DNA, 76 bp, Homo sapiens
  source 1..76: mol_type = unassigned DNA
gccaacgccatgcccaggccggagagtcccggggaggctgctggtgggcagctgactgcggggacactgggtggaa

SEQ ID NO: 204 - DNA, 76 bp, Homo sapiens
  source 1..76: mol_type = unassigned DNA
aggccaccagaggccaacgccatgcccaggccggagagtcccggggaggctgctggtggggaggcgaacgcgggga

SEQ ID NO: 205 - DNA, 76 bp, Homo sapiens
  source 1..76: mol_type = unassigned DNA
tgcccaggccggagagtcccggggcggctgctgggggggagctgactgggggggcactgggggggagacccgggcc

SEQ ID NO: 206 - DNA, 76 bp, Homo sapiens
  source 1..76: mol_type = unassigned DNA
taggcccttaaaacaactcgttccctcagaccacacacaagacagttcaagagggactcaaggacttacaggaatg

SEQ ID NO: 207 - DNA, 76 bp, Homo sapiens
  source 1..76: mol_type = unassigned DNA
actcaaggacttacaggaatgtccagtgctcccaagaaatcgaactccacaagcttggcttcccgcggacgtcctg

SEQ ID NO: 208 - DNA, 76 bp, Homo sapiens
  source 1..76: mol_type = unassigned DNA
aggcccttaaaacaactcgttccctcagaccacacacaagacagttcaagagggactcaaggacttacaggaatgt

SEQ ID NO: 209 - DNA, 76 bp, Homo sapiens
  source 1..76: mol_type = unassigned DNA
gccctcccagaggcccaccttcaagcagctggtggaggacctggaccgtgtccttaccgtgacgtccaccgacgtg

SEQ ID NO: 210 - DNA, 76 bp, Homo sapiens
  source 1..76: mol_type = unassigned DNA
gccgcgccctcccagaggcccaccttcaagcagctggtggaggacctggaccgtgtccttaccgtgacgtccaccg

SEQ ID NO: 211 - DNA, 76 bp, Homo sapiens
  source 1..76: mol_type = unassigned DNA
ggtggaggacctggaccgtgaccttaccgggacgtccaccgacgggagtgctggctctggcctggtgccacccgcc

SEQ ID NO: 212 - DNA, 76 bp, Homo sapiens
  source 1..76: mol_type = unassigned DNA
gacgtccaccgacgtgagtgctggctctggcctggtgccacccgcctatgcccctccccctgccgtccccggccat

SEQ ID NO: 213 - DNA, 76 bp, Homo sapiens
  source 1..76: mol_type = unassigned DNA
tgtccttaccgtgacgtccaccgacgtgagtgctggctctggcctggtgccacccgcctatgcccctccccctgcc

SEQ ID NO: 214 - DNA, 76 bp, Homo sapiens
  source 1..76: mol_type = unassigned DNA
tacctgctggtctcggtggccacgggcactggtctaccagggctgtccctccggagggggtcaaacttgagggata

SEQ ID NO: 215 - DNA, 76 bp, Homo sapiens
  source 1..76: mol_type = unassigned DNA
ctggaccgtgtccttaccgtgacgtccaccgacgtgagtgctggctctggcctggtgccacccgcccatgcccctc

SEQ ID NO: 216 - DNA, 76 bp, Homo sapiens
  source 1..76: mol_type = unassigned DNA
tgtccttaccgtgacgtccaccgacgtgagtgctggctctggcctggtgccacccgcctatgcccctcccctgccc

SEQ ID NO: 217 - DNA, 76 bp, Homo sapiens
  source 1..76: mol_type = unassigned DNA
aaaagatttaagtttagatctttaatatacctagaacggtggctgtaaccagcaaggcaggagccctttgtgttgg

SEQ ID NO: 218 - DNA, 76 bp, Homo sapiens
  source 1..76: mol_type = unassigned DNA
tgggtcaaacttgaggtataaggactgcttcctcaaggccgactccttatactggggacaagagggcaagtgatca

SEQ ID NO: 219 - DNA, 76 bp, Homo sapiens
  source 1..76: mol_type = unassigned DNA
gagtgctggctctggcctggtgccacccgcctatgcccctccccctggcgtccccggccatcctgccccccagagt

SEQ ID NO: 220 - DNA, 76 bp, Homo sapiens
  source 1..76: mol_type = unassigned DNA
gagtgctggctctggcctggtgccacccgcctatgcccctccccctgccgtccccggccatcctgccccccagagt

SEQ ID NO: 221 - DNA, 76 bp, Homo sapiens
  source 1..76: mol_type = unassigned DNA
gccaacgccatgcccaggccggagagtcccggggaggctgctggtggggagctgacttcggggacactggggggaa

SEQ ID NO: 222 - DNA, 76 bp, Homo sapiens
  source 1..76: mol_type = unassigned DNA
catgcgggagtgctggcatggcgcgccctcccagcggcccaccttcaagcagctggtgggggacctggaccgtgtc

SEQ ID NO: 223 - DNA, 76 bp, Homo sapiens
  source 1..76: mol_type = unassigned DNA
acgtgagtgctggctctggcctggtgccacccgcctatgcccctccccctgccgtccccggccatcctgcccccca

SEQ ID NO: 224 - DNA, 76 bp, Homo sapiens
  source 1..76: mol_type = unassigned DNA
ccctcccagaggcccaccttcaagcagctggtggaggacctggaccgtgtccttaccgtgacgtccaccgacgtga

SEQ ID NO: 225 - DNA, 76 bp, synthetic construct; identical to SEQ ID NO: 193
  misc_feature 1..76: note = Description of Artificial Sequence: Synthetic oligonucleotide
  source 1..76: mol_type = other DNA

SEQ ID NO: 226 - DNA, 76 bp, synthetic construct; identical to SEQ ID NO: 114
  misc_feature 1..76: note = Description of Artificial Sequence: Synthetic oligonucleotide
  source 1..76: mol_type = other DNA

SEQ ID NO: 227 - DNA, 76 bp, synthetic construct; identical to SEQ ID NO: 114
  misc_feature 1..76: note = Description of Artificial Sequence: Synthetic oligonucleotide
  source 1..76: mol_type = other DNA

SEQ ID NO: 228 - DNA, 76 bp, synthetic construct; identical to SEQ ID NO: 114
  misc_feature 1..76: note = Description of Artificial Sequence: Synthetic oligonucleotide
  source 1..76: mol_type = other DNA

SEQ ID NO: 229 - DNA, 76 bp, synthetic construct; identical to SEQ ID NO: 116
  misc_feature 1..76: note = Description of Artificial Sequence: Synthetic oligonucleotide
  source 1..76: mol_type = other DNA

SEQ ID NO: 230 - DNA, 76 bp, synthetic construct; identical to SEQ ID NO: 117
  misc_feature 1..76: note = Description of Artificial Sequence: Synthetic oligonucleotide
  source 1..76: mol_type = other DNA

SEQ ID NO: 231 - DNA, 76 bp, synthetic construct
  misc_feature 1..76: note = Description of Artificial Sequence: Synthetic oligonucleotide
  source 1..76: mol_type = other DNA
caggaatgtccagtgctaccaagaaatcgaactccacaagcttgggttcccgcggacgtcctccgggatggccgtg

SEQ ID NO: 232 - DNA, 76 bp, synthetic construct
  misc_feature 1..76: note = Description of Artificial Sequence: Synthetic oligonucleotide
  source 1..76: mol_type = other DNA
caggaatgtccagtgctcccaagaaatcgaactccacaagcttggcttcccgcggacgtcctgagggatggccggg

SEQ ID NO: 233 - DNA, 76 bp, synthetic construct; identical to SEQ ID NO: 120
  misc_feature 1..76: note = Description of Artificial Sequence: Synthetic oligonucleotide
  source 1..76: mol_type = other DNA

SEQ ID NO: 234 - DNA, 76 bp, synthetic construct; identical to SEQ ID NO: 121
  misc_feature 1..76: note = Description of Artificial Sequence: Synthetic oligonucleotide
  misc_difference 21: note = modified_base - a, c, t, g, unknown or other
  source 1..76: mol_type = other DNA

SEQ ID NO: 235 - DNA, 76 bp, synthetic construct; identical to SEQ ID NO: 122
  misc_feature 1..76: note = Description of Artificial Sequence: Synthetic oligonucleotide
  source 1..76: mol_type = other DNA

SEQ ID NO: 236 - DNA, 76 bp, synthetic construct; identical to SEQ ID NO: 123
  misc_feature 1..76: note = Description of Artificial Sequence: Synthetic oligonucleotide
  source 1..76: mol_type = other DNA

SEQ ID NO: 237 - DNA, 76 bp, synthetic construct
  misc_feature 1..76: note = Description of Artificial Sequence: Synthetic oligonucleotide
  source 1..76: mol_type = other DNA
tgctcccaagaaatcgaactccacaagcttggcttcccgcggacgtcctgagggatggccggggacggcaggggga

SEQ ID NO: 238 - DNA, 76 bp, synthetic construct
  misc_feature 1..76: note = Description of Artificial Sequence: Synthetic oligonucleotide
  source 1..76: mol_type = other DNA
aagaaatcgaactccacaagcttggcttcccgcggacgtcctgagggatggccggggacggcagggggaggggcat

SEQ ID NO: 239 - DNA, 76 bp, synthetic construct
  misc_feature 1..76: note = Description of Artificial Sequence: Synthetic oligonucleotide
  source 1..76: mol_type = other DNA
aaatcgaactccacaagcttggcttcccgcggacgtcctgagggatggccgggggcggcagggggaggggcatagg

SEQ ID NO: 240 - DNA, 76 bp, synthetic construct; identical to SEQ ID NO: 127
  misc_feature 1..76: note = Description of Artificial Sequence: Synthetic oligonucleotide
  source 1..76: mol_type = other DNA

SEQ ID NO: 241 - DNA, 76 bp, synthetic construct
  misc_feature 1..76: note = Description of Artificial Sequence: Synthetic oligonucleotide
  source 1..76: mol_type = other DNA
tcgtcccgcggacttcctgatggatcgccggggacggcagggggaggggcataggcgtgtggcaccaggccagctc

SEQ ID NO: 242 - DNA, 76 bp, synthetic construct
  misc_feature 1..76: note = Description of Artificial Sequence: Synthetic oligonucleotide
  misc_difference 37: note = modified_base - a, c, t, g, unknown or other
  source 1..76: mol_type = other DNA
cttcccgcggacgtcctgagggatggccggggacggnagggggaggggcataggcgggtggcaccaggccagagcc

SEQ ID NO: 243 - DNA, 76 bp, synthetic construct; identical to SEQ ID NO: 130
  misc_feature 1..76: note = Description of Artificial Sequence: Synthetic oligonucleotide
  source 1..76: mol_type = other DNA

SEQ ID NO: 244 - DNA, 76 bp, synthetic construct
  misc_feature 1..76: note = Description of Artificial Sequence: Synthetic oligonucleotide
  source 1..76: mol_type = other DNA
gagggatggccggggacggcagggggaggggcataggcgggtggcaccaggccagagccagcactcacgtcggtgg

SEQ ID NO: 245 - DNA, 76 bp, synthetic construct
  misc_feature 1..76: note = Description of Artificial Sequence: Synthetic oligonucleotide
  source 1..76: mol_type = other DNA
ggacaagagggcaagtgatcaggtctgactgccatcccctaacacacacaggggggctaagggcaggggaggggca

SEQ ID NO: 246 - DNA, 76 bp, synthetic construct; identical to SEQ ID NO: 245
  misc_feature 1..76: note = Description of Artificial Sequence: Synthetic oligonucleotide
  source 1..76: mol_type = other DNA

SEQ ID NO: 247 - DNA, 76 bp, synthetic construct
  misc_feature 1..76: note = Description of Artificial Sequence: Synthetic oligonucleotide
  source 1..76: mol_type = other DNA
atgcccctcccctgcccttagcccccctgtgtgtgttaggggatggcagtcagacctgatcacttgccctcttgtc

SEQ ID NO: 248 - DNA, 76 bp, synthetic construct
  misc_feature 1..76: note = Description of Artificial Sequence: Synthetic oligonucleotide
  source 1..76: mol_type = other DNA
gcctatgcccctcccctgcccttagcccccctgtgtgtgttaggggatggcagtcagacctgatcacttgccctct

SEQ ID NO: 249 - DNA, 76 bp, synthetic construct
  misc_feature 1..76: note = Description of Artificial Sequence: Synthetic oligonucleotide
  source 1..76: mol_type = other DNA
gtgccacccgcctatgcccctcccctgcccttagcccccctgtgtgtgttaggggatggcagtcagacctgatcac

SEQ ID NO: 250 - DNA, 76 bp, synthetic construct
  misc_feature 1..76: note = Description of Artificial Sequence: Synthetic oligonucleotide
  source 1..76: mol_type = other DNA
tgactgccatcccctaacacacacaggggggctaagggcaggggaggggcataggcggggggcaccaggccagagc

SEQ ID NO: 251 - DNA, 76 bp, synthetic construct; identical to SEQ ID NO: 250
  misc_feature 1..76: note = Description of Artificial Sequence: Synthetic oligonucleotide
  source 1..76: mol_type = other DNA

SEQ ID NO: 252 - DNA, 76 bp, synthetic construct
  misc_feature 1..76: note = Description of Artificial Sequence: Synthetic oligonucleotide
  source 1..76: mol_type = other DNA
tgccatcccctaacacacacaggggggctaagggcaggggaggggcataggcggggggcaccaggacagaggcagc

SEQ ID NO: 253 - DNA, 76 bp, synthetic construct
  misc_feature 1..76: note = Description of Artificial Sequence: Synthetic oligonucleotide
  source 1..76: mol_type = other DNA
cacacaggggggctaagggcaggggaggggcataggcgggggggaccaggcccgagccagcactcacgtcgggggg

SEQ ID NO: 254 - DNA, 76 bp, synthetic construct
  misc_feature 1..76: note = Description of Artificial Sequence: Synthetic oligonucleotide
  source 1..76: mol_type = other DNA
gacgtccaccgacgtgagtgctggctctggcctggtgccacccgcctatgcccctcccctgcccttagaccccctg

SEQ ID NO: 255 - DNA, 76 bp, synthetic construct; identical to SEQ ID NO: 195
  misc_feature 1..76: note = Description of Artificial Sequence: Synthetic oligonucleotide
  source 1..76: mol_type = other DNA

SEQ ID NO: 256 - DNA, 76 bp, synthetic construct; identical to SEQ ID NO: 216
  misc_feature 1..76: note = Description of Artificial Sequence: Synthetic oligonucleotide
  source 1..76: mol_type = other DNA

SEQ ID NO: 257 - DNA, 76 bp, synthetic construct
  misc_feature 1..76: note = Description of Artificial Sequence: Synthetic oligonucleotide
  source 1..76: mol_type = other DNA
cgggggtgggagtgtgcgggtgaccgggggtgggagtgtgcaggtgacctccctggcccttagccccctgcactct

SEQ ID NO: 258 - DNA, 76 bp, synthetic construct
  misc_feature 1..76: note = Description of Artificial Sequence: Synthetic oligonucleotide
  source 1..76: mol_type = other DNA
cgggtgaccgggggagggagtgtgcaggggacctccctggcccttagccccctgcactctggggggcaggatggcc

SEQ ID NO: 259 - DNA, 76 bp, synthetic construct
  misc_feature 1..76: note = Description of Artificial Sequence: Synthetic oligonucleotide
  source 1..76: mol_type = other DNA
ggagtgtgcaggtgacctccctggcccttagccccctgcactctggggggcaggatggccggggacggcaggggga

SEQ ID NO: 260 - DNA, 76 bp, synthetic construct
  misc_feature 1..76: note = Description of Artificial Sequence: Synthetic oligonucleotide
  source 1..76: mol_type = other DNA
ggggaggctgctggtgggcagctgactgcggggacactgggaggaagcctggaccctcagcgaacttcgcccagcc

SEQ ID NO: 261 - DNA, 76 bp, synthetic construct
  misc_feature 1..76: note = Description of Artificial Sequence: Synthetic oligonucleotide
  source 1..76: mol_type = other DNA
acagcctgggcacagaggtggctgtgcgaaggtcgctgagggtccaggcttccacccagtgtccccgcagtcagct

SEQ ID NO: 262 - DNA, 76 bp, synthetic construct
  misc_feature 1..76: note = Description of Artificial Sequence: Synthetic oligonucleotide
  source 1..76: mol_type = other DNA
tgactgcggggacactgggtggaagcctggaccctcagcgaccttcgcacagccacctctgtgcccaggctgtgcc

SEQ ID NO: 263 - DNA, 76 bp, synthetic construct
  misc_feature 1..76: note = Description of Artificial Sequence: Synthetic oligonucleotide
  source 1..76: mol_type = other DNA
cggggacactgggtggaagcctggaccctcagcgaccttcgcacagccacctctgtgcccaggctgtgccccagaa

SEQ ID NO: 264 - DNA, 76 bp, synthetic construct
  misc_feature 1..76: note = Description of Artificial Sequence: Synthetic oligonucleotide
  source 1..76: mol_type = other DNA
ggggacactgggtggaagcctggaccctcagcgaccttcgcacagccacctctgtggccaggctgtgccacagaag

SEQ ID NO: 265 - DNA, 76 bp, synthetic construct
  misc_feature 1..76: note = Description of Artificial Sequence: Synthetic oligonucleotide
  source 1..76: mol_type = other DNA
ggacactgggtggaagcctggaccctcagcgaccttcgcacagccacctctgtgcccaggctgtgccccagaaggc

SEQ ID NO: 266 - DNA, 76 bp, synthetic construct
  misc_feature 1..76: note = Description of Artificial Sequence: Synthetic oligonucleotide
  source 1..76: mol_type = other DNA
gaagcctggaccctcagcgaccttcgcacagccacctctgtgccccggctgtgccccagccggcccgccccacacc

SEQ ID NO: 267 - DNA, 76 bp, synthetic construct
  misc_feature 1..76: note = Description of Artificial Sequence: Synthetic oligonucleotide
  source 1..76: mol_type = other DNA
gaagcctggaccctcagcgaccttcgcacagccacctctgtgcccaggctgtgccccagaaggcccgccccacacc

SEQ ID NO: 268 - DNA, 76 bp, synthetic construct
  misc_feature 1..76: note = Description of Artificial Sequence: Synthetic oligonucleotide
  source 1..76: mol_type = other DNA
tggaccctcagcgaccttcgcacagccacctctgtgcccaggctgtgccccagaaggcccgccccacacctcagca

SEQ ID NO: 269 - DNA, 76 bp, synthetic construct
  misc_feature 1..76: note = Description of Artificial Sequence: Synthetic oligonucleotide
  source 1..76: mol_type = other DNA
gaccctcagcgaccttcgcacagccacctctgtgcccaggctgtgccccagaaggcccgccccacacctcagcact

SEQ ID NO: 270 - DNA, 76 bp, synthetic construct
  misc_feature 1..76: note = Description of Artificial Sequence: Synthetic oligonucleotide
  source 1..76: mol_type = other DNA
tcagcgaccttcgcacagccacctctgtgcccaggctgtgccccagaaggcccgccccacacctcagcactctggg

SEQ ID NO: 271 - DNA, 76 bp, synthetic construct
  misc_feature 1..76: note = Description of Artificial Sequence: Synthetic oligonucleotide
  source 1..76: mol_type = other DNA
ccttcgcacagccacctctgtgcccaggctgtgccccagaaggcccgccccacacctcagcactctggggggcagg

SEQ ID NO: 272 - DNA, 76 bp, synthetic construct; identical to SEQ ID NO: 212
  misc_feature 1..76: note = Description of Artificial Sequence: Synthetic oligonucleotide
  source 1..76: mol_type = other DNA

SEQ ID NO: 273 - DNA, 76 bp, synthetic construct
  misc_feature 1..76: note = Description of Artificial Sequence: Synthetic oligonucleotide
  source 1..76: mol_type = other DNA
caagagggactcaaggacttacaggaatgtccagtgctcccaagaaatcgaactccacaagcttggcttcccgcgg

SEQ ID NO: 274 - DNA, 76 bp, synthetic construct; identical to SEQ ID NO: 273
  misc_feature 1..76: note = Description of Artificial Sequence: Synthetic oligonucleotide
  source 1..76: mol_type = other DNA

SEQ ID NO: 275 - DNA, 76 bp, synthetic construct
  misc_feature 1..76: note = Description of Artificial Sequence: Synthetic oligonucleotide
  source 1..76: mol_type = other DNA
ataggcccttaaaacaactcgttccctcagaccacacacaagacagttcaagagggactcaaggacttacaggaat

SEQ ID NO: 276 - DNA, 76 bp, synthetic construct
  misc_feature 1..76: note = Description of Artificial Sequence: Synthetic oligonucleotide
  source 1..76: mol_type = other DNA
tcaagagggactcaaggacttacaggaatgtccagtgctcccaagaaatcgaactccacaagcttggcttcccgcg

SEQ ID NO: 277 - DNA, 76 bp, synthetic construct
  misc_feature 1..76: note = Description of Artificial Sequence: Synthetic oligonucleotide
  source 1..76: mol_type = other DNA
accacacacaagacagttcaagagggactcaaggacttacaggaatgtccagtgctcccaagaaatcgaactccac

SEQ ID NO: 278 - DNA, 76 bp, synthetic construct; identical to SEQ ID NO: 128
  misc_feature 1..76: note = Description of Artificial Sequence: Synthetic oligonucleotide
  source 1..76: mol_type = other DNA

SEQ ID NO: 279 - DNA, 76 bp, synthetic construct; identical to SEQ ID NO: 131
  misc_feature 1..76: note = Description of Artificial Sequence: Synthetic oligonucleotide
  source 1..76: mol_type = other DNA

SEQ ID NO: 280 - DNA, 76 bp, synthetic construct
  misc_feature 1..76: note = Description of Artificial Sequence: Synthetic oligonucleotide
  source 1..76: mol_type = other DNA
caagagcctcagacagtgcatgagggacccgagacagtgcggcgagggaacagcacagcggccccatgcccccaac

SEQ ID NO: 281 - DNA, 76 bp, synthetic construct
  misc_feature 1..76: note = Description of Artificial Sequence: Synthetic oligonucleotide
  source 1..76: mol_type = other DNA
caagagcctcagacagtgcatgagggacccgagacagtgcggcgagggaacagcacaggggccccatgcccccaac

SEQ ID NO: 282 - DNA, 76 bp, synthetic construct
  misc_feature 1..76: note = Description of Artificial Sequence: Synthetic oligonucleotide
  source 1..76: mol_type = other DNA
cgttccctcagaccacacacaagacagttcaagagggactcaaggacttacaggaatgtccagtgctcccaagaga

SEQ ID NO: 283 - DNA, 76 bp, synthetic construct
  misc_feature 1..76: note = Description of Artificial Sequence: Synthetic oligonucleotide
  source 1..76: mol_type = other DNA
ccaggaatagaaaatataggcccttaaaacaactcgttccctcagaccacacacaagacagttcaagagggactca

SEQ ID NO: 284 - DNA, 76 bp, synthetic construct; identical to SEQ ID NO: 129
  misc_feature 1..76: note = Description of Artificial Sequence: Synthetic oligonucleotide
  misc_difference 40: note = modified_base - a, c, t, g, unknown or other
  source 1..76: mol_type = other DNA

SEQ ID NO: 285 - DNA, 76 bp, synthetic construct
  misc_feature 1..76: note = Description of Artificial Sequence: Synthetic oligonucleotide
  source 1..76: mol_type = other DNA
gccctgcccgcaggtacatgatcatgcgggagtgctggcatgccgcgccctcccagaggcccaccttcaagcagct

SEQ ID NO: 286 - DNA, 76 bp, synthetic construct
  misc_feature 1..76: note = Description of Artificial Sequence: Synthetic oligonucleotide
  source 1..76: mol_type = other DNA
ctggcatgccgcgccctcccagaggcccacctttaagcagctggtagagggcctggaccgtgtccttaccgtgacg

SEQ ID NO: 287 - DNA, 76 bp, synthetic construct
  misc_feature 1..76: note = Description of Artificial Sequence: Synthetic oligonucleotide
  source 1..76: mol_type = other DNA
taaaacaactcgttccctcagaccacacacaagacagttcaagagggactcaaggacttacaggaatgtccagtgc

SEQ ID NO: 288 - DNA, 76 bp, synthetic construct; identical to SEQ ID NO: 118
  misc_feature 1..76: note = Description of Artificial Sequence: Synthetic oligonucleotide
  source 1..76: mol_type = other DNA

SEQ ID NO: 289 - DNA, 76 bp, synthetic construct; identical to SEQ ID NO: 124
  misc_feature 1..76: note = Description of Artificial Sequence: Synthetic oligonucleotide
  source 1..76: mol_type = other DNA

SEQ ID NO: 290 - DNA, 76 bp, synthetic construct
  misc_feature 1..76: note = Description of Artificial Sequence: Synthetic oligonucleotide
  source 1..76: mol_type = other DNA
agaccacacacaagacagttcaagagggactcaaggacttacaggaatgtccagtgctcccaagaaatcgaactcc

SEQ ID NO: 291 - DNA, 76 bp, synthetic construct; identical to SEQ ID NO: 119
  misc_feature 1..76: note = Description of Artificial Sequence: Synthetic oligonucleotide
  source 1..76: mol_type = other DNA

SEQ ID NO: 292 - DNA, 76 bp, synthetic construct
  misc_feature 1..76: note = Description of Artificial Sequence: Synthetic oligonucleotide
  source 1..76: mol_type = other DNA
ggcatgccgcgccctcccagaggcccaccttcaagcagctggtggaggacctggaccgtgtccttaccgtgacgtc

SEQ ID NO: 293 - DNA, 76 bp, synthetic construct; identical to SEQ ID NO: 292
  misc_feature 1..76: note = Description of Artificial Sequence: Synthetic oligonucleotide
  source 1..76: mol_type = other DNA

SEQ ID NO: 294 - DNA, 76 bp, synthetic construct
  misc_feature 1..76: note = Description of Artificial Sequence: Synthetic oligonucleotide
  source 1..76: mol_type = other DNA
cggcgcacatacctgctggtctcggtggccacgggcactggtctaccaggactgtccctcaggagggggtcaaact

SEQ ID NO: 295 - DNA, 76 bp, synthetic construct
  misc_feature 1..76: note = Description of Artificial Sequence: Synthetic oligonucleotide
  source 1..76: mol_type = other DNA
atacctgctggtctcggtggccacgggcactggtctaccaggactgtccctcaggagggggtcaaacttgaggtat

SEQ ID NO: 296 - DNA, 76 bp, synthetic construct
  misc_feature 1..76: note = Description of Artificial Sequence: Synthetic oligonucleotide
  source 1..76: mol_type = other DNA
aggtataaggactgcttcctcaaggccgactccttaaactggggacaagagggcaagtgatcaggtctgactgcca

SEQ ID NO: 297 - DNA, 76 bp, synthetic construct
  misc_feature 1..76: note = Description of Artificial Sequence: Synthetic oligonucleotide
  source 1..76: mol_type = other DNA
ggaggacctggactgtgtccttaccgtgacgtccaccgacgtgagtgctggctctggcctggtgccacccgcctat

SEQ ID NO: 298 - DNA, 76 bp, synthetic construct
  misc_feature 1..76: note = Description of Artificial Sequence: Synthetic oligonucleotide
  source 1..76: mol_type = other DNA
ggaggacctggaccgtgtccttaccgtgacgtccaccgacgtgagtgctggctctggcctggtgccacccgcctat

SEQ ID NO: 299 - DNA, 76 bp, synthetic construct
  misc_feature 1..76: note = Description of Artificial Sequence: Synthetic oligonucleotide
  source 1..76: mol_type = other DNA
caagcagctggtggaggacctggaccgtgtccttaccgtgacgtccaccgacgtgagtgctggctctggcctggtg

SEQ ID NO: 300 - DNA, 76 bp, synthetic construct
  misc_feature 1..76: note = Description of Artificial Sequence: Synthetic oligonucleotide
  source 1..76: mol_type = other DNA
accttcaagcagctggtggaggacctggaccgtgtccttaccgtgacgtccaccgacgtgagtgctggctctggcc

SEQ ID NO: 301 - DNA, 76 bp, synthetic construct
  misc_feature 1..76: note = Description of Artificial Sequence: Synthetic oligonucleotide
  source 1..76: mol_type = other DNA
caaacttgaggtataaggactgcttcctcaaggccgactccttaaactggggacaagagggcaagtgatcaggtct

SEQ ID NO: 302 - DNA, 76 bp, synthetic construct; identical to SEQ ID NO: 214
  misc_feature 1..76: note = Description of Artificial Sequence: Synthetic oligonucleotide
  source 1..76: mol_type = other DNA

SEQ ID NO: 303 - DNA, 76 bp, synthetic construct; identical to SEQ ID NO: 197
  misc_feature 1..76: note = Description of Artificial Sequence: Synthetic oligonucleotide
  source 1..76: mol_type = other DNA

SEQ ID NO: 304 - DNA, 76 bp, synthetic construct; identical to SEQ ID NO: 189
  misc_feature 1..76: note = Description of Artificial Sequence: Synthetic oligonucleotide
  source 1..76: mol_type = other DNA

SEQ ID NO: 305 - DNA, 76 bp, synthetic construct
  misc_feature 1..76: note = Description of Artificial Sequence: Synthetic oligonucleotide
  source 1..76: mol_type = other DNA
gcgccctcccagaggcccaccttcaagcagctggtggaggacctggaccgtgtccttaccgtgacgtccaccgacg

SEQ ID NO: 306 - DNA, 76 bp, synthetic construct
  misc_feature 1..76: note = Description of Artificial Sequence: Synthetic oligonucleotide
  source 1..76: mol_type = other DNA
gcgggagtgctggcatgccgcgccctcccagaggcccaccttcaagcagctggtggaggacctggaccgtgtcctt

SEQ ID NO: 307 - DNA, 76 bp, synthetic construct
  misc_feature 1..76: note = Description of Artificial Sequence: Synthetic oligonucleotide
  source 1..76: mol_type = other DNA
cctccactgggtcctcaggggtgggggtccctccggggctgggcgggggagggactggcaggcctgcaggggggtt

SEQ ID NO: 308 - DNA, 76 bp, synthetic construct
  misc_feature 1..76: note = Description of Artificial Sequence: Synthetic oligonucleotide
  source 1..76: mol_type = other DNA
tcacggcagcaagaaccacactcactgctgcaaggccaccagaggccaacgccatgcccaggccggagagtcccgg

SEQ ID NO: 309 - DNA, 76 bp, synthetic construct
  misc_feature 1..76: note = Description of Artificial Sequence: Synthetic oligonucleotide
  source 1..76: mol_type = other DNA
tacatgatcatgcgggagggctggcatgccgcgccctcccagaggcccaccttcaagcagctggtggagggccggg

SEQ ID NO: 310 - DNA, 76 bp, synthetic construct
  misc_feature 1..76: note = Description of Artificial Sequence: Synthetic oligonucleotide
  source 1..76: mol_type = other DNA
ggtgggaagcggcggggctcactcctgagcgccctgcccgcagggacatgatcatgcgggggtgctggccttgcgg

SEQ ID NO: 311 - DNA, 76 bp, synthetic construct; identical to SEQ ID NO: 305
  misc_feature 1..76: note = Description of Artificial Sequence: Synthetic oligonucleotide
  source 1..76: mol_type = other DNA

SEQ ID NO: 312 - DNA, 76 bp, synthetic construct; identical to SEQ ID NO: 107
  misc_feature 1..76: note = Description of Artificial Sequence: Synthetic oligonucleotide
  source 1..76: mol_type = other DNA

SEQ ID NO: 313 - DNA, 76 bp, synthetic construct
  misc_feature 1..76: note = Description of Artificial Sequence: Synthetic oligonucleotide
  source 1..76: mol_type = other DNA
gcaggtacatgatcatgcgggagtgccggcatttcgggaccttccctcgggccaccctcttccggttgttgtgggc

SEQ ID NO: 314 - DNA, 76 bp, synthetic construct
  misc_feature 1..76: note = Description of Artificial Sequence: Synthetic oligonucleotide
  source 1..76: mol_type = other DNA
gcaggtacatgatcatgcgggagtgctggcatgccgcgccctccccgaggaccaccttccagcagccgggggaggg

SEQ ID NO: 315 - DNA, 76 bp, synthetic construct
  misc_feature 1..76: note = Description of Artificial Sequence: Synthetic oligonucleotide
  source 1..76: mol_type = other DNA
cccgaataaggtgggaagcggcggggctcactcctgagcgccctgaccgcaggtacatgagcatgcgggagtggcg

SEQ ID NO: 316 - DNA, 76 bp, synthetic construct
  misc_feature 1..76: note = Description of Artificial Sequence: Synthetic oligonucleotide
  source 1..76: mol_type = other DNA
cgtgtccttaccgtgacgtccaccgacgtgagtgctggctctggcctggtgccacccgcctatgcccctccccctg

SEQ ID NO: 317 - DNA, 76 bp, synthetic construct
  misc_feature 1..76: note = Description of Artificial Sequence: Synthetic oligonucleotide
  source 1..76: mol_type = other DNA
acatgatcatgcgggagtgctggcatgccgcgcccccccagaggcccaccttcaagcagctggtggaggacctgga

SEQ ID NO: 318 - DNA, 76 bp, synthetic construct
  misc_feature 1..76: note = Description of Artificial Sequence: Synthetic oligonucleotide
  source 1..76: mol_type = other DNA
gccttctggggcacagcctgggcacagaggtggctgtgcgaaggtcgctgagggtccaggcttccacccagtgtcc

SEQ ID NO: 319 - DNA, 27 bp, synthetic construct
  misc_feature 1..27: note = Description of Artificial Sequence: Synthetic oligonucleotide
  source 1..27: mol_type = other DNA
  CDS 2..25
tccggatgaactggtccctgtccttcg

SEQ ID NO: 320 - protein, 8 aa, synthetic construct
  REGION 1..8: note = Description of Artificial Sequence: Synthetic peptide
PDELVPVL

SEQ ID NO: 321 - DNA, 26 bp, synthetic construct
  misc_feature 1..26: note = Description of Artificial Sequence: Synthetic oligonucleotide
  source 1..26: mol_type = other DNA
  CDS 1..24
ctgatcgacctggatgtccccgtcga

SEQ ID NO: 322 - protein, 8 aa, synthetic construct
  REGION 1..8: note = Description of Artificial Sequence: Synthetic peptide
LIDLDVPV

SEQ ID NO: 323 - DNA, 26 bp, synthetic construct
  misc_feature 1..26: note = Description of Artificial Sequence: Synthetic oligonucleotide
  source 1..26: mol_type = other DNA
  CDS 3..26
atgctgtcagagggttttgtgatgaa

SEQ ID NO: 324 - protein, 8 aa, synthetic construct
  REGION 1..8: note = Description of Artificial Sequence: Synthetic peptide
AVRGFCDE

SEQ ID NO: 325 - DNA, 27 bp, synthetic construct
  misc_feature 1..27: note = Description of Artificial Sequence: Synthetic oligonucleotide
  source 1..27: mol_type = other DNA
  CDS 3..26
actacgataaaagacataaccagtgcg

SEQ ID NO: 326 - protein, 8 aa, synthetic construct
  REGION 1..8: note = Description of Artificial Sequence: Synthetic peptide
YDKRHNQC

SEQ ID NO: 327 - DNA, 176 bp, synthetic construct
  misc_feature 1..176: note = Description of Artificial Sequence: Synthetic polynucleotide
  source 1..176: mol_type = other DNA
cgggggtggccccccctcgggggacagcgcatgcccgctgcgcaccatcaagagagtccagttcggagtcctgagtccggatgaactggtccctgtccttcgaatggtggaaggtgataccatctatgattactgctggtattctctgatgtcctcagcccagccagacacctcct

SEQ ID NO: 328 - protein, 59 aa, synthetic construct
  REGION 1..59: note = Description of Artificial Sequence: Synthetic polypeptide
  VARIANT 59: note = MOD_RES - Any amino acid
GGGPPSGDSACPLRTIKRVQFGVLSPDELVPVLRMVEGDTIYDYCWYSLMSSAQPDTSX

SEQ ID NO: 329 - DNA, 90 bp, synthetic construct
  misc_feature 1..90: note = Description of Artificial Sequence: Synthetic oligonucleotide
  source 1..90: mol_type = other DNA
tggtccctgtccttcgaatggtggaaggtgataccatctatgattactgctggtattctctgatgtcctcagcccagccagacacctcct

SEQ ID NO: 330 - DNA, 90 bp, synthetic construct
  misc_feature 1..90: note = Description of Artificial Sequence: Synthetic oligonucleotide
  source 1..90: mol_type = other DNA
ctggtccctgtccttcgaatggtggaaggtgataccatctatgattactgctggtattctctgatgtcctcagcccggccagacacctcc

SEQ ID NO: 331 - DNA, 90 bp, synthetic construct
  misc_feature 1..90: note = Description of Artificial Sequence: Synthetic oligonucleotide
  source 1..90: mol_type = other DNA
actggtccctgtccttcgaatggtggaaggtgataccatctatgattactgctggtattctctgatgtcctcagcccagccagacaccac

SEQ ID NO: 332 - DNA, 90 bp, synthetic construct
  misc_feature 1..90: note = Description of Artificial Sequence: Synthetic oligonucleotide
  source 1..90: mol_type = other DNA
aactggtccctgtccttcgaatggtggaaggtgataccatctatgattactgctggtattctctgatgtcctcagcccagccagacacct

SEQ ID NO: 333 - DNA, 90 bp, synthetic construct
  misc_feature 1..90: note = Description of Artificial Sequence: Synthetic oligonucleotide
  source 1..90: mol_type = other DNA
gaactggtccctgtccttcgaatggtggaaggtgataccatctatgattactgctggtattctctgatgtcctcagcccagccagacacc

SEQ ID NO: 334 - DNA, 90 bp, synthetic construct
  misc_feature 1..90: note = Description of Artificial Sequence: Synthetic oligonucleotide
  source 1..90: mol_type = other DNA
tgaactggtccctgtccttcgaatggtggaaggtgataccatctatgattactgctggtattctctgatgtcctcagcccagccagacac

SEQ ID NO: 335 - DNA, 91 bp, synthetic construct
  misc_feature 1..91: note = Description of Artificial Sequence: Synthetic oligonucleotide
  source 1..91: mol_type = other DNA
atgaactggtccctgtccttcgaatggtggaaggtgataccatctatgattactgctgtattctctgatgtcctcagcccagccagacacc

SEQ ID NO: 336 - DNA, 90 bp, synthetic construct
  misc_feature 1..90: note = Description of Artificial Sequence: Synthetic oligonucleotide
  source 1..90: mol_type = other DNA
gatgaactggtccctgtccttcgaatggtggaaggtgataccatctatgattactgctggtattctctgatgtcctcagcccagccagac

SEQ ID NO: 337 - DNA, 90 bp, synthetic construct
  misc_feature 1..90: note = Description of Artificial Sequence: Synthetic oligonucleotide
  source 1..90: mol_type = other DNA
ggatgaactggtccctgtccttcgaatggtggaaggtgataccatctatgattactgctggtattctctgatgtcctcagcccagccaga

SEQ ID NO: 338 - DNA, 90 bp, synthetic construct
  misc_feature 1..90: note = Description of Artificial Sequence: Synthetic oligonucleotide
  source 1..90: mol_type = other DNA
cggatgaactggtccctgtccttcgaatggtggaaggtgataccatctatgattactgctggtattctctgatgtcctcagcccagccag

SEQ ID NO: 339 - DNA, 90 bp, synthetic construct
  misc_feature 1..90: note = Description of Artificial Sequence: Synthetic oligonucleotide
  source 1..90: mol_type = other DNA
ccggatgaactggtccctgtccttcgaatggtggaaggtgataccatctatgattactgctggtattctctgatgtcctcagcccagcca

SEQ ID NO: 340 - DNA, 90 bp, synthetic construct
  misc_feature 1..90: note = Description of Artificial Sequence: Synthetic oligonucleotide
  source 1..90: mol_type = other DNA
tccggatgaactggtccctgtccttcgaatggtggaaggtgataccatctatgattactgctggtattctctgatgtcctcagcccagcc

SEQ ID NO: 341 - DNA, 90 bp, synthetic construct
  misc_feature 1..90: note = Description of Artificial Sequence: Synthetic oligonucleotide
  source 1..90: mol_type = other DNA
gtccggaggaactggtccctgtccttcgaatggtggaaggtgataccatctatgattgctgctggtattctctgatgtcctcagcccagc

SEQ ID NO: 342 - DNA, 90 bp, synthetic construct
  misc_feature 1..90: note = Description of Artificial Sequence: Synthetic oligonucleotide
  source 1..90: mol_type = other DNA
agtccggatgaactggtccctgtccttcgaatggtggaaggtgataccatctatgattactgctggtattctctgatgtcctcagcccag

SEQ ID NO: 343 - DNA, 90 bp, synthetic construct
  misc_feature 1..90: note = Description of Artificial Sequence: Synthetic oligonucleotide
  source 1..90: mol_type = other DNA
gagtccggatgaactggtccctgtccttcgaatggtggaaggtgataccatctatgattactgctggtattctctgatgtcctcagcccg

SEQ ID NO: 344 - DNA, 90 bp, synthetic construct
  misc_feature 1..90: note = Description of Artificial Sequence: Synthetic oligonucleotide
  source 1..90: mol_type = other DNA
tgagtccggatgaactggtccctgtccttcgaatggtggaaggtgataccatctatgattactgctggtattctctgatgtcctcagccc

SEQ ID NO: 345 - DNA, 90 bp, synthetic construct
  misc_feature 1..90: note = Description of Artificial Sequence: Synthetic oligonucleotide
  source 1..90: mol_type = other DNA
ctgagtccggatgaactggtccctgtccttcgaatggtggaaggtgataccatctatgattactgctggtattctctgatgtcctcagcc

SEQ ID NO: 346 - DNA, 90 bp, synthetic construct
  misc_feature 1..90: note = Description of Artificial Sequence: Synthetic oligonucleotide
  source 1..90: mol_type = other DNA
cctgagtccggatgaactggtccctgtccttcgaatggtggaaggtgataccatctatgattactgctggtattctctgatgtcctcagc

SEQ ID NO: 347 - DNA, 90 bp, synthetic construct
  misc_feature 1..90: note = Description of Artificial Sequence: Synthetic oligonucleotide
  source 1..90: mol_type = other DNA
tcctgagtccggatgaactggtccctgtccttcgaatggtggaaggtgataccatctatgattactgctggtattctctgatgtcctcag

SEQ ID NO: 348 - DNA, 90 bp, synthetic construct
  misc_feature 1..90: note = Description of Artificial Sequence: Synthetic oligonucleotide
  source 1..90: mol_type = other DNA
gtcctgagtccggatgaactggtccctgtccttcgaatggtggaaggtgataccatctatgattactgctggtattctctgatgtcctca

SEQ ID NO: 349 - DNA, 90 bp, synthetic construct
  misc_feature 1..90: note = Description of Artificial Sequence: Synthetic oligonucleotide
  source 1..90: mol_type = other DNA
agtcctgagtccggatgaactggtccctgtccttcgaatggtggaaggtgataccatctatgattactgctggtattctctgatgtcctc

SEQ ID NO: 350 - DNA, 90 bp, synthetic construct
  misc_feature 1..90: note = Description of Artificial Sequence: Synthetic oligonucleotide
  source 1..90: mol_type = other DNA
gagtcctgagtccggatgaactggtccctgtccttcgaatggtggaaggtgataccatctatgattactgctggtattctctgatgtcct

SEQ ID NO: 351 - DNA, 90 bp, synthetic construct
  misc_feature 1..90: note = Description of Artificial Sequence: Synthetic oligonucleotide
  source 1..90: mol_type = other DNA
ggagtcctgagtccggatgaactggtccctgtccttcgaatggtggaaggtgataccatctatgattactgctggtattctctgatgtcc

SEQ ID NO: 352 - DNA, 90 bp, synthetic construct
  misc_feature 1..90: note = Description of Artificial Sequence: Synthetic oligonucleotide
  source 1..90: mol_type = other DNA
cggagtcctgagtccggatgaactggtccctgtccttcgaatggtggaaggtgataccatctatgattactgctggtattctctgatgtc

SEQ ID NO: 353 - DNA, 90 bp, synthetic construct
  misc_feature 1..90: note = Description of Artificial Sequence: Synthetic oligonucleotide
  source 1..90: mol_type = other DNA
tcggagtcctgagtccggatgaactggtccctgtccttcgaatggtggaaggtgataccatctatgattactgctggtattctctgatgt

SEQ ID NO: 354 - DNA, 90 bp, synthetic construct
  misc_feature 1..90: note = Description of Artificial Sequence: Synthetic oligonucleotide
  source 1..90: mol_type = other DNA
ttcggagtcctgagtccggatgaactggtccctgtccttcgaatggtggaaggtgataccatctatgattactgctggtattctctgatg

SEQ ID NO: 355 - DNA, 90 bp, synthetic construct
  misc_feature 1..90: note = Description of Artificial Sequence: Synthetic oligonucleotide
  source 1..90: mol_type = other DNA
gttcggagtcctgagtccggatgaactggtccctgtccttcgaatggtggaaggtgataccatctatgattactgctggtattctctgat

SEQ ID NO: 356 - DNA, 90 bp, synthetic construct
  misc_feature 1..90: note = Description of Artificial Sequence: Synthetic oligonucleotide
  source 1..90: mol_type = other DNA
cagttcggagtcctgagtccggatgaactggtccctgtccttcgaatggtggaaggtgataccatctatgattactgctggtattctctg

SEQ ID NO: 357 - DNA, 90 bp, synthetic construct
  misc_feature 1..90: note = Description of Artificial Sequence: Synthetic oligonucleotide
  source 1..90: mol_type = other DNA
ccagttcggagtcctgagtccggatgaactggtccctgtccttcgaatggtggaaggtgataccatctatgattactgctggtattctct

SEQ ID NO: 358 - DNA, 90 bp, synthetic construct
  misc_feature 1..90: note = Description of Artificial Sequence: Synthetic oligonucleotide
  source 1..90: mol_type = other DNA
tccagttcggagtcctgagtccggatgaactggtccctgtccttcgaatggtggaaggtgataccatctatgattactgctggtattctc

SEQ ID NO: 359 - DNA, 90 bp, synthetic construct
  misc_feature 1..90: note = Description of Artificial Sequence: Synthetic oligonucleotide
  source 1..90: mol_type = other DNA
gtccagttcggagtcctgagtccggatgaactggtccctgtccttcgaatggtggaaggtgataccatctatgattactgctggtattct

SEQ ID NO: 360 - DNA, 90 bp, synthetic construct
  misc_feature 1..90: note = Description of Artificial Sequence: Synthetic oligonucleotide
  source 1..90: mol_type = other DNA
gagtccagttcggagtcctgagtccggatgaactggtccctgtccttcgaatggtggaaggtgataccatctatgattactgctggtatt

SEQ ID NO: 361 - DNA, 90 bp, synthetic construct
  misc_feature 1..90: note = Description of Artificial Sequence: Synthetic oligonucleotide
  source 1..90: mol_type = other DNA
agagtccagttcggagtcctgagtccggatgaactggtccctgtccttcgaatggtggaaggtgataccatctatgattactgctggtat

SEQ ID NO: 362 - DNA, 90 bp, synthetic construct
  misc_feature 1..90: note = Description of Artificial Sequence: Synthetic oligonucleotide
  source 1..90: mol_type = other DNA
gagagtccagttcggagtcctgagtccggatgaactggtccctgtccttcgaatggtggaaggtgataccatctatgattactgctggta

SEQ ID NO: 363 - DNA, 90 bp, synthetic construct
  misc_feature 1..90: note = Description of Artificial Sequence: Synthetic oligonucleotide
  source 1..90: mol_type = other DNA
agagagtccagttcggagtcctgagtccggatgaactggtccctgtccttcgaatggtggaaggtgataccatctatgattactgctggt

SEQ ID NO: 364 - DNA, 90 bp, synthetic construct
  misc_feature 1..90: note = Description of Artificial Sequence: Synthetic oligonucleotide
  source 1..90: mol_type = other DNA
aagagagtccagttcggagtcctgagtccggatgaactggtccctgtccttcgaatggtggaaggggataccatctatgattactgccgg

SEQ ID NO: 365 - DNA, 90 bp, synthetic construct
  misc_feature 1..90: note = Description of Artificial Sequence: Synthetic oligonucleotide
  source 1..90: mol_type = other DNA
caagagagtccagttcggagtcctgagtccggatgaactggtccctgtccttcgaatggtggaaggtgataccatctatgattactgctg

SEQ ID NO: 366 - DNA, 90 bp, synthetic construct
  misc_feature 1..90: note = Description of Artificial Sequence: Synthetic oligonucleotide
  source 1..90: mol_type = other DNA
tcaagagagtccagttcggagtcctgagtccggatgaactggtccctgtccttcgaatggtggaaggtgataccatctatgattactgct

SEQ ID NO: 367 - DNA, 90 bp, synthetic construct
  misc_feature 1..90: note = Description of Artificial Sequence: Synthetic oligonucleotide
  source 1..90: mol_type = other DNA
atcaagagagtccagttcggagtcctgagtccggatgaactggtccctgtccttcgaatggtggaaggtgataccatctatgattactgc

SEQ ID NO: 368 - DNA, 90 bp, synthetic construct
  misc_feature 1..90: note = Description of Artificial Sequence: Synthetic oligonucleotide
  source 1..90: mol_type = other DNA
caccatcaagagagtccagttcggagtcctgagtccggatgaactggtccctgtccttcgaatggtggaaggtgataccatctatgatta

SEQ ID NO: 369 - DNA, 90 bp, synthetic construct
  misc_feature 1..90: note = Description of Artificial Sequence: Synthetic oligonucleotide
  source 1..90: mol_type = other DNA
cgcaccatcaagagagtccagttcggagtcctgagtccggatgaactggtccctgtccttcgaatggtggaaggtgataccatctatgat

SEQ ID NO: 370 - DNA, 90 bp, synthetic construct
  misc_feature 1..90: note = Description of Artificial Sequence: Synthetic oligonucleotide
  source 1..90: mol_type = other DNA
gcgcaccatcaagagagtccagttcggagtcctgagtccggatgaactggtccctgtccttcgaatggtggaaggtgataccatctatga

SEQ ID NO: 371 - DNA, 90 bp, synthetic construct
  misc_feature 1..90: note = Description of Artificial Sequence: Synthetic oligonucleotide
  source 1..90: mol_type = other DNA
gcccgctgcgcaccatcaagagagtccagttcggagtcctgagtccggatgaactggtccctgtccttcgaatggtggaaggtgatacca

SEQ ID NO: 372 - DNA, 90 bp, synthetic construct
  misc_feature 1..90: note = Description of Artificial Sequence: Synthetic oligonucleotide
  source 1..90: mol_type = other DNA
cgcatgcccgctgcgcaccatcaagagagtccagttcggagtcctgagtccggatgaactggtccctgtccttcgaatggtggaaggtga

SEQ ID NO: 373 - DNA, 90 bp, synthetic construct
  misc_feature 1..90: note = Description of Artificial Sequence: Synthetic oligonucleotide
  source 1..90: mol_type = other DNA
gcgcatgcccgctgcgcaccatcaagagagtccagttcggagtcctgagtccggatgaactggtccctgtccttcgaatggtggaaggtg

SEQ ID NO: 374 - DNA, 90 bp, synthetic construct
  misc_feature 1..90: note = Description of Artificial Sequence: Synthetic oligonucleotide
  source 1..90: mol_type = other DNA
agcgcatgcccgctgcgcaccatcaagagagtccagttcggagtcctgagtccggatgaactggtccctgtccttcgaatggtggaaggt

SEQ ID NO: 375 - DNA, 90 bp, synthetic construct
  misc_feature 1..90: note = Description of Artificial Sequence: Synthetic oligonucleotide
  source 1..90: mol_type = other DNA
cagcgcatgcccgctgcgcaccatcaagagagtccagttcggagtcctgagtccggatgaactggtccctgtccttcgaatggtggaagg

SEQ ID NO: 376 - DNA, 90 bp, synthetic construct
  misc_feature 1..90: note = Description of Artificial Sequence: Synthetic oligonucleotide
  source 1..90: mol_type = other DNA
gggacagcgcatgcccgctgcgcaccatcaagagagtccagttcggagtcctgagtccggatgaactggtccctgtccttcgaatggtgg

SEQ ID NO: 377 - DNA, 90 bp, synthetic construct
  misc_feature 1..90: note = Description of Artificial Sequence: Synthetic oligonucleotide
  source 1..90: mol_type = other DNA
cgggggacagcgcatgcccgctgcgcaccatcaagagagtccagttcggagtcctgagtccggatgaactggtccctgtccttcgaatgg

SEQ ID NO: 378 - DNA, 90 bp, synthetic construct
  misc_feature 1..90: note = Description of Artificial Sequence: Synthetic oligonucleotide
  source 1..90: mol_type = other DNA
cccccctcgggggacagcgcatgcccgctgcgcaccatcaagagagtccagttcggagtcctgagtccggatgaactggtccctgtcctt

SEQ ID NO: 379 - DNA, 90 bp, synthetic construct
  misc_feature 1..90: note = Description of Artificial Sequence: Synthetic oligonucleotide
  source 1..90: mol_type = other DNA
ccccccctcgggggacagcgcatgcccgctgcgcaccatcaagagagtccagttcggagtcctgagtccggatgaactggtccctgtcct

SEQ ID NO: 380 - DNA, 90 bp, synthetic construct
  misc_feature 1..90: note = Description of Artificial Sequence: Synthetic oligonucleotide
  source 1..90: mol_type = other DNA
ggccccccctcgggggacagcgcatgcccgctgcgcaccatcaagagagtccagttcggagtcctgagtccggatgaactggtccctgtc

SEQ ID NO: 381 - DNA, 90 bp, synthetic construct
  misc_feature 1..90: note = Description of Artificial Sequence: Synthetic oligonucleotide
  source 1..90: mol_type = other DNA
ggggtggccccccctcgggggacagcgcatgcccgctgcgcaccatcaagagagtccagttcggagtcctgagtccggatgaactggtcc

SEQ ID NO: 382 - DNA, 90 bp, synthetic construct
  misc_feature 1..90: note = Description of Artificial Sequence: Synthetic oligonucleotide
  source 1..90: mol_type = other DNA
cgggggtggccccccctcgggggacagcgcatgcccgctgcgcaccatcaagagagtccagttcggagtcctgagtccggatgaactggt

SEQ ID NO: 383 - DNA, 170 bp, synthetic construct
  misc_feature 1..170: note = Description of Artificial Sequence: Synthetic polynucleotide
  source 1..170: mol_type = other DNA
  CDS 2..169
gcagctggactgtgccttggacctaatgaggcgcctgcctccccagcaaatcgagaaaaacctcagcgacctgatcgacctggatgtccccgttgaggccctcaccacggtgaagccatactgcaatgagatccatgcccaggctcaactgtggctcaagagagacccca

SEQ ID NO: 384 - protein, 56 aa, synthetic construct
  REGION 1..56: note = Description of Artificial Sequence: Synthetic polypeptide
QLDCALDLMRRLPPQQIEKNLSDLIDLDVPVEALTTVKPYCNEIHAQAQLWLKRDP

SEQ ID NO: 385 - DNA, 90 bp, synthetic construct
  misc_feature 1..90: note = Description of Artificial Sequence: Synthetic oligonucleotide
  source 1..90: mol_type = other DNA
tggatgtccccgtgggggccctcaccacggtgaagccatactgcaatgagatccatgcccaggctcaactgtggctcaagagagacccca

SEQ ID NO: 386 - DNA, 90 bp, synthetic construct
  misc_feature 1..90: note = Description of Artificial Sequence: Synthetic oligonucleotide
  source 1..90: mol_type = other DNA
ctggatctccccgtcgagcccctccccacggtgaagccatactgcaatgagatccatgcccaggctcaactgtggctcaagagagacccc

SEQ ID NO: 387 - DNA, 90 bp, synthetic construct
  misc_feature 1..90: note = Description of Artificial Sequence: Synthetic oligonucleotide
  source 1..90: mol_type = other DNA
caaggatgtccccgttgaggccctcaccacggtgaagccatactgcaatgagatccatgcccaggctcaactgtggctcaagagagacct

SEQ ID NO: 388 - DNA, 90 bp, synthetic construct
  misc_feature 1..90: note = Description of Artificial Sequence: Synthetic oligonucleotide
  source 1..90: mol_type = other DNA
acctggatgtccccgtcgaggccctccccacggtgaagccatactgcaatgagatccatgcccaggctcaactgtggctcaagagagacc

SEQ ID NO: 389 - DNA, 90 bp, synthetic construct
  misc_feature 1..90: note = Description of Artificial Sequence: Synthetic oligonucleotide
  source 1..90: mol_type = other DNA
gccctggatgtccccgtcgaggccctcaccacggtgaagccatactgcaatgagatccatgcccaggctcaactgtggctcaagagagac

SEQ ID NO: 390 - DNA, 90 bp, synthetic construct
  misc_feature 1..90: note = Description of Artificial Sequence: Synthetic oligonucleotide
  source 1..90: mol_type = other DNA
ccaccttgatgtccccgtcgaggccctcaccacggtgaagccatactgcaatgagatccatgcccaggctcaactgtggctcaagagaga

SEQ ID NO: 391 - DNA, 90 bp, synthetic construct
  misc_feature 1..90: note = Description of Artificial Sequence: Synthetic oligonucleotide
  source 1..90: mol_type = other DNA
gcgacctggatgtccccgtcgaggccctccccacggtgaagccatactgcaatgagatccatgcccaggctcaactgtggctcaagagag

SEQ ID NO: 392 - DNA, 90 bp, synthetic construct
  misc_feature 1..90: note = Description of Artificial Sequence: Synthetic oligonucleotide
  source 1..90: mol_type = other DNA
gatcgacctggatgtccccgtcgaggccctcaccacggtgaagccatactgcaatgagatccatgcccaggctcaactgtggctcaagag

SEQ ID NO: 393 - DNA, 90 bp, synthetic construct
  misc_feature 1..90: note = Description of Artificial Sequence: Synthetic oligonucleotide
  source 1..90: mol_type = other DNA
acctgatcgacctggatgtccccgtcgaggccctcaccacggtgaagccatactgcaatgagatccatgcccaggctcaactgtggctca

SEQ ID NO: 394 - DNA, 90 bp, synthetic construct
  misc_feature 1..90: note = Description of Artificial Sequence: Synthetic oligonucleotide
  source 1..90: mol_type = other DNA
cgacctgatcgacctggatgtccccgtcgaggccctcaccacggtgaagccatactgcaatgagatccatgcccaggctcaactgtggct

SEQ ID NO: 395 - DNA, 90 bp, synthetic construct
  misc_feature 1..90: note = Description of Artificial Sequence: Synthetic oligonucleotide
  source 1..90: mol_type = other DNA
ctcagcgacctgatcgacctggatgtccccgtcgaggccctcaccacggtgaagccatactgcaatgagatccatgcccaggctcaactg

SEQ ID NO: 396 - DNA, 90 bp, synthetic construct
  misc_feature 1..90: note = Description of Artificial Sequence: Synthetic oligonucleotide
  source 1..90: mol_type = other DNA
aaaacctcagcgacctgatcgacctggatgtccccgtcgaggccctcaccacggtgaagccatactgcaatgagatccatgcccaggctc

SEQ ID NO: 397 - DNA, 90 bp, synthetic construct
  misc_feature 1..90: note = Description of Artificial Sequence: Synthetic oligonucleotide
  source 1..90: mol_type = other DNA
gaaaaacctcagcgacctgatcgacctggatgtccccgtcgaggccctcaccacggtgaagccatactgcaatgagatccatgcccaggc

SEQ ID NO: 398 - DNA, 90 bp, synthetic construct
  misc_feature 1..90: note = Description of Artificial Sequence: Synthetic oligonucleotide
  source 1..90: mol_type = other DNA
agaaaaacctcagcgacctgatcgacctggatgtccccgtcgaggccctcaccacggtgaagccatactgcaatgagatccatgcccagg

SEQ ID NO: 399 - DNA, 90 bp, synthetic construct
  misc_feature 1..90: note = Description of Artificial Sequence: Synthetic oligonucleotide
  source 1..90: mol_type = other DNA
cgagaaaaacctcagcgacctgatcgacctggatgtccccgtcgaggccctcaccacggtgaagccatactgcaatgagatccatgccca

SEQ ID NO: 400 - DNA, 90 bp, synthetic construct
  misc_feature 1..90: note = Description of Artificial Sequence: Synthetic oligonucleotide
  source 1..90: mol_type = other DNA
tcgagaaaaacctcagcgacctgatcgacctggatgtccccgtcgaggccctcaccacggtgaagccatactgcaatgagatccatgccc

SEQ ID NO: 401 - DNA, 90 bp, synthetic construct
  misc_feature 1..90: note = Description of Artificial Sequence: Synthetic oligonucleotide
  source 1..90: mol_type = other DNA
atcgagaaaaacctcagcgacctgatcgacctggatgtccccgtcgaggccctcaccacggtgaagccatactgcaatgagatccatgcc

SEQ ID NO: 402 - DNA, 90 bp, synthetic construct
  misc_feature 1..90: note = Description of Artificial Sequence: Synthetic oligonucleotide
  source 1..90: mol_type = other DNA
aatcgagaaaaacctcagcgacctgatcgacctggatgtccccgtcgaggccctcaccacggtgaagccatactgcaatgagatccatgc

SEQ ID NO: 403 - DNA, 90 bp, synthetic construct
  misc_feature 1..90: note = Description of Artificial Sequence: Synthetic oligonucleotide
  source 1..90: mol_type = other DNA
aaatcgaaaaaaacctcagcgacctgatcgacctggatggccccggcgaggccctcaccacggtgaagccatactgcaatgagatccatg

SEQ ID NO: 404 - DNA, 90 bp, synthetic construct
  misc_feature 1..90: note = Description of Artificial Sequence: Synthetic oligonucleotide
  source 1..90: mol_type = other DNA
caaatcgagaaaaacctcagcgacctgatcgacctggatgtccccgtcgaggccctcaccacggtgaagccatactgcaatgagatccat

SEQ ID NO: 405 - DNA, 90 bp, synthetic construct
  misc_feature 1..90: note = Description of Artificial Sequence: Synthetic oligonucleotide
  source 1..90: mol_type = other DNA
gcaaatcgagaaaaacctcagcgacctgatcgacctggatgtccccgtcgaggccctcaccacggtgaagccatactgcaatgagatcca

SEQ ID NO: 406 - DNA, 90 bp, synthetic construct
  misc_feature 1..90: note = Description of Artificial Sequence: Synthetic oligonucleotide
  source 1..90: mol_type = other DNA
agcaaatcgagaaaaacctcagcgacctgatcgacctggatgtccccgtcgaggccctcaccacggtgaagccatactgcaatgagatcc

SEQ ID NO: 407 - DNA, 90 bp, synthetic construct
  misc_feature 1..90: note = Description of Artificial Sequence: Synthetic oligonucleotide
  source 1..90: mol_type = other DNA
cagcaaatcgagaaaaacctcagcgacctgatcgacctggatgtccccgtcgaggccctcaccacggtgaagccatactgcaatgagatc

SEQ ID NO: 408 - DNA, 90 bp, synthetic construct
  misc_feature 1..90: note = Description of Artificial Sequence: Synthetic oligonucleotide
  source 1..90: mol_type = other DNA
ccccagcaaatcgagaaaaacctcagcgacctgatcgacctggatgtccccgtcgaggccctcaccacggtgaagccatactgcaatgag

SEQ ID NO: 409 - DNA, 90 bp, synthetic construct
  misc_feature 1..90: note = Description of Artificial Sequence: Synthetic oligonucleotide
  source 1..90: mol_type = other DNA
ctccccagcaaatcgagaaaaacctcagcgacctgatcgacctggatgtccccgtcgaggccctcaccacggtgaagccatactgcaatg

SEQ ID NO: 410 - DNA, 90 bp, synthetic construct
  misc_feature 1..90: note = Description of Artificial Sequence: Synthetic oligonucleotide
  source 1..90: mol_type = other DNA
cctccccagcaaatcgagaaaaacctcagcgacctgatcgacctggatgtccccgtcgaggccctcaccacggtgaagccatactgcaat

SEQ ID NO: 411 - DNA, 90 bp, synthetic construct
  misc_feature 1..90: note = Description of Artificial Sequence: Synthetic oligonucleotide
  source 1..90: mol_type = other DNA
gcctccccagcaaatcgagaaaaacctcagcgacctgatcgacctggatgtccccgtcgaggccctcaccacggtgaagccatactgcaa

SEQ ID NO: 412 - DNA, 90 bp, synthetic construct
  misc_feature 1..90: note = Description of Artificial Sequence: Synthetic oligonucleotide
  source 1..90: mol_type = other DNA
tgcctccccagcaaatcgagaaaaacctcagcgacccgatcgacctggatgtccccgtcgaggccctcaccacggtgaagccatactgca

SEQ ID NO: 413 - DNA, 90 bp, synthetic construct
  misc_feature 1..90: note = Description of Artificial Sequence: Synthetic oligonucleotide
  source 1..90: mol_type = other DNA
ctgcctccccagcaaatcgagaaaaacctcagcgacctgatcgacctggatgtccccgtcgaggccctcaccacggtgaagccatactgc

SEQ ID NO: 414 - DNA, 90 bp, synthetic construct
  misc_feature 1..90: note = Description of Artificial Sequence: Synthetic oligonucleotide
  source 1..90: mol_type = other DNA
cctgcctccccagcaaatcgagaaaaacctcagcgacctgatcgacctggatgtccccgtcgaggccctcaccacggtgaagccatactg

SEQ ID NO: 415 - DNA, 90 bp, synthetic construct
  misc_feature 1..90: note = Description of Artificial Sequence: Synthetic oligonucleotide
  source 1..90: mol_type = other DNA
cgcctgcctccccagcaaatcgagaaaaacctcagcgacctgatcgacctggatgtccccgtcgaggccctcaccacggtgaagccatac

SEQ ID NO: 416 - DNA, 90 bp, synthetic construct
  misc_feature 1..90: note = Description of Artificial Sequence: Synthetic oligonucleotide
  source 1..90: mol_type = other DNA
ggcgcctgcctccccagcaaatcgagaaaaacctcagcgacctgatcgacctggatgtccccgtcgaggccctcaccacggtgaagccat

SEQ ID NO: 417 - DNA, 90 bp, synthetic construct
  misc_feature 1..90: note = Description of Artificial Sequence: Synthetic oligonucleotide
  source 1..90: mol_type = other DNA
gcggtgcctgcctccccagcaaatcgagaaaaacctcagcgacctgatcgacctggatgtccccgtcgaggccctcaccacggtgaagcc

SEQ ID NO: 418 - DNA, 90 bp, synthetic construct
  misc_feature 1..90: note = Description of Artificial Sequence: Synthetic oligonucleotide
  source 1..90: mol_type = other DNA
tgaggcgcctgcctccccagcaaatcgagaaaaacctcagcgacctgatcgacctggatgtccccgtcgaggccctcaccacggtgaagc

SEQ ID NO: 419 - DNA, 90 bp, synthetic construct
  misc_feature 1..90: note = Description of Artificial Sequence: Synthetic oligonucleotide
  source 1..90: mol_type = other DNA
atgaggcgcctgcctccccagcaaatcgagaaaaacctcagcgacctgatcgacctggatgtccccgtcgaggccctcaccacggtgaag

SEQ ID NO: 420 - DNA, 90 bp, synthetic construct
  misc_feature 1..90: note = Description of Artificial Sequence: Synthetic oligonucleotide
  source 1..90: mol_type = other DNA
aatgaggcgcctgcctccccagcaaatcgagaaaaacctcagcgacctgatcgacctggatgtccccgtcgaggccctcaccacggtgaa

SEQ ID NO: 421 - DNA, 90 bp, synthetic construct
  misc_feature 1..90: note = Description of Artificial Sequence: Synthetic oligonucleotide
  source 1..90: mol_type = other DNA
taatgaggcgcctgcctccccagcaaatcgagaaaaacctcagcgacctgatcgacctggatgtccccgtcgaggccctcaccacggtga

SEQ ID NO: 422 - DNA, 90 bp, synthetic construct
  misc_feature 1..90: note = Description of Artificial Sequence: Synthetic oligonucleotide
  source 1..90: mol_type = other DNA
gacctaatgaggcgcctgcctccccagcaaatcgagaaaaacctcagcgacctgatcgacctggatgtccccgtcgaggccctcaccacg

SEQ ID NO: 423 - DNA, 90 bp, synthetic construct
  misc_feature 1..90: note = Description of Artificial Sequence: Synthetic oligonucleotide
  source 1..90: mol_type = other DNA
ttggacctaatgaggcgcctgcctccccagcaaatcgagaaaaacctcagcgacctgatcgacctggatgtccccgtcgaggccctcacc

SEQ ID NO: 424 - DNA, 90 bp, synthetic construct
  misc_feature 1..90: note = Description of Artificial Sequence: Synthetic oligonucleotide
  source 1..90: mol_type = other DNA
ctgggacctaaggaggcgcctgcctccccagcaaatcgagaaaaacctcagcgacctgatcgacctggatgtccccgtcgaggccctcac

SEQ ID NO: 425 - DNA, 90 bp, synthetic construct
  misc_feature 1..90: note = Description of Artificial Sequence: Synthetic oligonucleotide
  source 1..90: mol_type = other DNA
gccttggacctaatgaggcgcctgcctccccagcaaatcgagaaaaacctcagcgacctgatcgacctggatgtccccgtcgaggccctc

SEQ ID NO: 426 - DNA, 90 bp, synthetic construct
  misc_feature 1..90: note = Description of Artificial Sequence: Synthetic oligonucleotide
  source 1..90: mol_type = other DNA
gtgccttggacctaatgaggcgcctgcctccccagcaaatcgagaaaaacctcagcgacctgatcgacctggatgtccccgtcgaggccc

SEQ ID NO: 427 - DNA, 90 bp, synthetic construct
  misc_feature 1..90: note = Description of Artificial Sequence: Synthetic oligonucleotide
  source 1..90: mol_type = other DNA
actgggcctggggcctaatgaggcgcctgcctccccagcaaatcgagaaaaacctcagcgacctgatcgacctggatgtccccgtcgagg

SEQ ID NO: 428 - DNA, 90 bp, synthetic construct
  misc_feature 1..90: note = Description of Artificial Sequence: Synthetic oligonucleotide
  source 1..90: mol_type = other DNA
gactgtgcctgggccctaaggaggcgcctgcctccccagcaaatcgagaaaaacctcagcgacctgatcgacctggatgtccccgtcgag

SEQ ID NO: 429 - DNA, 90 bp, synthetic construct
  misc_feature 1..90: note = Description of Artificial Sequence: Synthetic oligonucleotide
  source 1..90: mol_type = other DNA
catggactgtgccttggacctaatgaggcgcctgcctccccagcaaatcgagaaaaacctcagcgacctgatcgacctggatgtccccga

SEQ ID NO: 430 - DNA, 90 bp, synthetic construct
  misc_feature 1..90: note = Description of Artificial Sequence: Synthetic oligonucleotide
  source 1..90: mol_type = other DNA
gctggactgtgccttggacctaatgaggccccttcctccccagcaaatcgagaaaaacctcagcgacctgatcgacctggatgtcccccg

SEQ ID NO: 431 - DNA, 91 bp, synthetic construct
  misc_feature 1..91: note = Description of Artificial Sequence: Synthetic oligonucleotide
  source 1..91: mol_type = other DNA
agctggactgtgccttggacctaatgaggcgcctgcctccccagcaaatcgagaaaaaccccagcgacctgatcgacctggatgtccccgg

SEQ ID NO: 432 - DNA, 91 bp, synthetic construct
  misc_feature 1..91: note = Description of Artificial Sequence: Synthetic oligonucleotide
  source 1..91: mol_type = other DNA
gcagctggactgtgccttggacctaatgaggcgcctgcctccccagcaaatcgagaaaaacctcagcgacctgatcgacctggatgtcccc

SEQ ID NO: 433 - DNA, 176 bp, synthetic construct
  misc_feature 1..176: note = Description of Artificial Sequence: Synthetic polynucleotide
  source 1..176: mol_type = other DNA
ggagtgggttaatgcattaatccttaagaataaactgaaagtgcgaactgcctatccgtcattgagacttattcatgctgtcagagggttttgtgatgaaggaacctgtacagataaagccaatattctgtatgcctgggcgagaaatgctccccctacccggctccccaaaggtg

SEQ ID NO: 434 - protein, 59 aa, synthetic construct
  REGION 1..59: note = Description of Artificial Sequence: Synthetic polypeptide
  VARIANT 59: note = MOD_RES - Any amino acid
EWVNALILKNKLKVRTAYPSLRLIHAVRGFCDEGTCTDKANILYAWARNAPPTRLPKGX

SEQ ID NO: 435 - DNA, 90 bp, synthetic construct
  misc_feature 1..90: note = Description of Artificial Sequence: Synthetic oligonucleotide
  source 1..90: mol_type = other DNA
ggttttgtgatgaaggaacctgtacagataaagccaatattctgtatgcctgggcgagaaatgctccccctacccggctccccaaaggtg

SEQ ID NO: 436 - DNA, 90 bp, synthetic construct
  misc_feature 1..90: note = Description of Artificial Sequence: Synthetic oligonucleotide
  source 1..90: mol_type = other DNA
tggttttgtgatgaaggaacctgtacagataaagccaatattctgtatgcctgggcgagaaatgctccccctacccggctccccaaaggt

SEQ ID NO: 437 - DNA, 90 bp, synthetic construct
  misc_feature 1..90: note = Description of Artificial Sequence: Synthetic oligonucleotide
  source 1..90: mol_type = other DNA
ctggttttgtgatgaaggaacctgtacagataaagccaatattctgtatgcctgggcgagaaatgctccccctacgcggctccccaaagg

SEQ ID NO: 438 - DNA, 90 bp, synthetic construct
  misc_feature 1..90: note = Description of Artificial Sequence: Synthetic oligonucleotide
  source 1..90: mol_type = other DNA
gagggttttgtgatgaaggaacctgtacagataaagccaatattctgtatgcctgggcgagaaatgctccccctacccggctccccaaag

SEQ ID NO: 439 - DNA, 91 bp, synthetic construct
  misc_feature 1..91: note = Description of Artificial Sequence: Synthetic oligonucleotide
  source 1..91: mol_type = other DNA
gttactggttttgtgatgaaggaacctgtacagataaagccaatattctgtatgcctgggcgagaaatgctccccctacccggctccccaa

SEQ ID NO: 440 - DNA, 90 bp, synthetic construct
  misc_feature 1..90: note = Description of Artificial Sequence: Synthetic oligonucleotide
  source 1..90: mol_type = other DNA
gctgtcagagggttttgtgatgaaggaacctgtacagataaagccaatattctgtatgcctgggcgagaaatgctccccctacccggctc

SEQ ID NO: 441 - DNA, 91 bp, synthetic construct
  misc_feature 1..91: note = Description of Artificial Sequence: Synthetic oligonucleotide
  source 1..91: mol_type = other DNA
tgctgtcagagggttttgtgatgaaggaacctgtacagataaagccaatattctgtatgcctgggcgagaaatgctccccctacccggccc

SEQ ID NO: 442 - DNA, 90 bp, synthetic construct
  misc_feature 1..90: note = Description of Artificial Sequence: Synthetic oligonucleotide
  source 1..90: mol_type = other DNA
atgctgtcagagggttttgtgatgaaggaacctgtacagataaagccaatattctgtatgcctgggcgagaaatgctccccctacccggc

SEQ ID NO: 443 - DNA, 90 bp, synthetic construct
  misc_feature 1..90: note = Description of Artificial Sequence: Synthetic oligonucleotide
  source 1..90: mol_type = other DNA
catgctgtcagagggttttgtgatgaaggagcctgtacagataaagccaatattctgtatgcctgggcgagaaatgctccccctacccgg

SEQ ID NO: 444 - DNA, 90 bp, synthetic construct
  misc_feature 1..90: note = Description of Artificial Sequence: Synthetic oligonucleotide
  source 1..90: mol_type = other DNA
tcatgctgtcagagggttttgtgatgaaggaacctgtacagataaagccaatattctgtatgcctgggcgagaaatgctccccctacccg

SEQ ID NO: 445 - DNA, 90 bp, synthetic construct
  misc_feature 1..90: note = Description of Artificial Sequence: Synthetic oligonucleotide
  source 1..90: mol_type = other DNA
tattcatgctgtcagagggttttgtgatgaaggaacctgtacagataaagccaatattctgtatgcctgggcgagaaatgctccccctac

SEQ ID NO: 446 - DNA, 90 bp, synthetic construct
  misc_feature 1..90: note = Description of Artificial Sequence: Synthetic oligonucleotide
  source 1..90: mol_type = other DNA
cttattcatgctgtcagagggttttgtgatgaaggaacctgtacagataaagccaatattctgtatgcctgggcgagaaatactccccct

SEQ ID NO: 447 - DNA, 90 bp, synthetic construct
  misc_feature 1..90: note = Description of Artificial Sequence: Synthetic oligonucleotide
  source 1..90: mol_type = other DNA
acttattcatgctgtcagagggttttgtgatgaaggaacctgtacagataaagccaatattctgtatgcctgggcgagaaatgctccccc

SEQ ID NO: 448 - DNA, 90 bp, synthetic construct
  misc_feature 1..90: note = Description of Artificial Sequence: Synthetic oligonucleotide
  source 1..90: mol_type = other DNA
gagacttattcatgctgtcagagggttttgtgatgaaggaacctgtacagataaagccaatattctgtatgcctgggcgagaaatgctcc

SEQ ID NO: 449 - DNA, 90 bp, synthetic construct
  misc_feature 1..90: note = Description of Artificial Sequence: Synthetic oligonucleotide
  source 1..90: mol_type = other DNA
tgagacttattcatgctgtcagagggttttgtgatgaaggaacctgtacagataaagccaatattctgtatgcctgggcgagaaatgctc

SEQ ID NO: 450 - DNA, 90 bp, synthetic construct
  misc_feature 1..90: note = Description of Artificial Sequence: Synthetic oligonucleotide
  source 1..90: mol_type = other DNA
ttgagacttattcatgctgtcagagggttttgtgatgaaggaacctgtacagataaagccaatattctgtatgcctgggcgagaaatgct

SEQ ID NO: 451 - DNA, 90 bp, synthetic construct
  misc_feature 1..90: note = Description of Artificial Sequence: Synthetic oligonucleotide
  source 1..90: mol_type = other DNA
gtcattgagacttattcatgctgtcagagggttttgtgatgaaggaacctgtacagataaagccaatattctgtatgcctgggcgagaaa

SEQ ID NO: 452 - DNA, 90 bp, synthetic construct
  misc_feature 1..90: note = Description of Artificial Sequence: Synthetic oligonucleotide
  source 1..90: mol_type = other DNA
ccgtcattgagacttattcatgctgtcagagggttttgtgatgaaggaacctgtacagataaagccaatattctgtatgcctgggcgaga

SEQ ID NO: 453 - DNA, 90 bp, synthetic construct
  misc_feature 1..90: note = Description of Artificial Sequence: Synthetic oligonucleotide
  source 1..90: mol_type = other DNA
tatccgtcattgagacttattcatgctgtcagagggttttgtgatgaaggaacctgtacagataaagccaatattctgtatgcctgggcg

SEQ ID NO: 454 - DNA, 90 bp, synthetic construct
  misc_feature 1..90: note = Description of Artificial Sequence: Synthetic oligonucleotide
  source 1..90: mol_type = other DNA
ctatccgtcattgagacttattcatgctgtcagagggttttgtgatgaaggaacctgtacagataaagccaatattctgtatgcctgggc

SEQ ID NO: 455 - DNA, 90 bp, synthetic construct
  misc_feature 1..90: note = Description of Artificial Sequence: Synthetic oligonucleotide
  source 1..90: mol_type = other DNA
cctatccgtcattgagacttattcatgctgtcagagggttttgtgatgaaggaacctgtacagataaagccaatattctgtatgcctggg

SEQ ID NO: 456 - DNA, 90 bp, synthetic construct
  misc_feature 1..90: note = Description of Artificial Sequence: Synthetic oligonucleotide
  source 1..90: mol_type = other DNA
gcctatccgtcattgagacttattcatgctgtcagagggttttgtgatgaaggaacctgtacagataaagccaatattctgtatgcctgg

SEQ ID NO: 457 - DNA, 90 bp, synthetic construct
  misc_feature 1..90: note = Description of Artificial Sequence: Synthetic oligonucleotide
  source 1..90: mol_type = other DNA
tgaaagtgcgaactgcctatccgtcattgagacttattcatgctgtcagagggttttgtgatgaaggaacctgtacagataaagccaata

SEQ ID NO: 458 - DNA, 90 bp, synthetic construct
  misc_feature 1..90: note = Description of Artificial Sequence: Synthetic oligonucleotide
  source 1..90: mol_type = other DNA
ctgaaagtgcgaactgcctatccgtcattgagacttattcatgctgtcagagggttttgtgatgaaggaacctgtacagataaagccaat

SEQ ID NO: 459 - DNA, 90 bp, synthetic construct
  misc_feature 1..90: note = Description of Artificial Sequence: Synthetic oligonucleotide
  source 1..90: mol_type = other DNA
aaactgaaagtgcgaactgcctatccgtcattgagacttattcatgctgtcagagggttttgtgatgaaggaacctgtacagataaagcc

SEQ ID NO: 460 - DNA, 90 bp, synthetic construct
  misc_feature 1..90: note = Description of Artificial Sequence: Synthetic oligonucleotide
  source 1..90: mol_type = other DNA
taaactgaaagtgcgaactgcctatccgtcattgagacttattcatgctgtcagagggttttgtgatgaaggaacctgtacagataaagc

SEQ ID NO: 461 - DNA, 90 bp, synthetic construct
  misc_feature 1..90: note = Description of Artificial Sequence: Synthetic oligonucleotide
  source 1..90: mol_type = other DNA
attaatccttaagaataaactgaaagtgcgaactgcctatccgtcattgagacttattcatgctgtcagagggttttgtgatgaaggaac

SEQ ID NO: 462 - DNA, 90 bp, synthetic construct
  misc_feature 1..90: note = Description of Artificial Sequence: Synthetic oligonucleotide
  source 1..90: mol_type = other DNA
gcattaatccttaagaataaactgaaagtgcgaactgcctatccgtcattgagacttattcatgctgtcagagggttttgtgatgaagga

SEQ ID NO: 463 - DNA, 90 bp, synthetic construct
  misc_feature 1..90: note = Description of Artificial Sequence: Synthetic oligonucleotide
  source 1..90: mol_type = other DNA
ggagtgggttaatgcattaatccttaagaataaactgaaagtgcgaactgcctatccgtcattgagacttattcatgctgtcagagggtt

SEQ ID NO: 464 - DNA, 167 bp, synthetic construct
  misc_feature 1..167: note = Description of Artificial Sequence: Synthetic polynucleotide
  source 1..167: mol_type = other DNA
acacgattttgtcttgtgttgccaagtctacctgtgccatcaacaacaccctcattgctttcttcattttgactacgataaaagacataaccagtgcggtgcaatccaagcgaagaaaatccaagtaaacaagcaggactgcgacttgatacttggaaatgtgtgtg

SEQ ID NO: 465 - protein, 56 aa, synthetic construct
  REGION 1..56: note = Description of Artificial Sequence: Synthetic polypeptide
  VARIANT 56: note = MOD_RES - Any amino acid
HDFVLCCQVYLCHQQHPHCFLHFDYDKRHNQCGAIQAKKIQVNKQDCDLILGNVCX

SEQ ID NO: 466 - DNA, 90 bp, synthetic construct
  misc_feature 1..90: note = Description of Artificial Sequence: Synthetic oligonucleotide
  source 1..90: mol_type = other DNA
ataaaagacataaccagtgcggtgcaatccaagcgaagaaaatccaagtaaacaagcaggactgcgacttgatacttggaaatgtgtgtg

SEQ ID NO: 467 - DNA, 90 bp, synthetic construct
  misc_feature 1..90: note = Description of Artificial Sequence: Synthetic oligonucleotide
  source 1..90: mol_type = other DNA
ttgctttcttcattttgactacgataaaagacataaccagtgcggtgcaatccaagcgaagaaaatccaagtaaacaagcaggactgcga

SEQ ID NO: 468 - DNA, 90 bp, synthetic construct
  misc_feature 1..90: note = Description of Artificial Sequence: Synthetic oligonucleotide
  source 1..90: mol_type = other DNA
cctcattgctttcttcattttgactacgataaaagacataaccagtgcggtgcaatccaagcgaagaaaatccaagtaaacaagcaggac

SEQ ID NO: 469 - DNA, 90 bp, synthetic construct
  misc_feature 1..90: note = Description of Artificial Sequence: Synthetic oligonucleotide
  source 1..90: mol_type = other DNA
ccctcattgctttcttcattttgactacgataaaagacataaccagtgcggtgcaatccaagcgaagaaaatccaagtaaacaagcagga

SEQ ID NO: 470 - DNA, 90 bp, synthetic construct
  misc_feature 1..90: note = Description of Artificial Sequence: Synthetic oligonucleotide
  source 1..90: mol_type = other DNA
caccctcattgctttcttcattttgactacgataaaagacataaccagtgcggtgcaatccaagcgaagaaaatccaagtaaacaagcag

SEQ ID NO: 471 - DNA, 90 bp, synthetic construct
  misc_feature 1..90: note = Description of Artificial Sequence: Synthetic oligonucleotide
  source 1..90: mol_type = other DNA
caacaccctcattgctttcttcattttgactacgataaaagacataaccagtgcggtgcaatccaagcgaagaaaatccaagtaaacaag

SEQ ID NO: 472 - DNA, 90 bp, synthetic construct
  misc_feature 1..90: note = Description of Artificial Sequence: Synthetic oligonucleotide
  source 1..90: mol_type = other DNA
caacaacaccctcattgctttcttcattttgactacgataaaagacataaccagtgcggtgcaatccaagcgaagaaaatccaagtaaac

SEQ ID NO: 473 - DNA, 90 bp, synthetic construct
  misc_feature 1..90: note = Description of Artificial Sequence: Synthetic oligonucleotide
  source 1..90: mol_type = other DNA
atcaacaacaccctcattgctttcttcattttgactacgataaaagacataaccagtgcggtgcaatccaagcgaagaaaatccaagtaa

SEQ ID NO: 474 - DNA, 90 bp, synthetic construct
  misc_feature 1..90: note = Description of Artificial Sequence: Synthetic oligonucleotide
  source 1..90: mol_type = other DNA
catcaacaacaccctcattgctttcttcattttgactacgataaaagacataaccagtgcggtgcaatccaagcgaagaaaatccaagta

SEQ ID NO: 475 - DNA, 90 bp, synthetic construct
  misc_feature 1..90: note = Description of Artificial Sequence: Synthetic oligonucleotide
  source 1..90: mol_type = other DNA
gtgccatcaacaacaccctcattgctttcttcattttgactacgataaaagacataaccagtgcggtgcaatccaagcgaagaaaatcca

SEQ ID NO: 476 - DNA, 90 bp, synthetic construct
  misc_feature 1..90: note = Description of Artificial Sequence: Synthetic oligonucleotide
  source 1..90: mol_type = other DNA
gtctacctgtgccatcaacaacaccctcattgctttcttcattttgactacgataaaagacataaccagtgcggtgcaatccaagcgaag

SEQ ID NO: 477 - DNA, 90 bp, synthetic construct
  misc_feature 1..90: note = Description of Artificial Sequence: Synthetic oligonucleotide
  source 1..90: mol_type = other DNA
caagtctacctgtgccatcaacaacaccctcattgctttcttcattttgactacgataaaagacataaccagtgcggtgcaatccaagcg

SEQ ID NO: 478 - DNA, 90 bp, synthetic construct
  misc_feature 1..90: note = Description of Artificial Sequence: Synthetic oligonucleotide
  source 1..90: mol_type = other DNA
ccaagtctacctgtgccatcaacaacaccctcattgctttcttcattttgactacgataaaagacataaccagtgcggtgcaatccaagc

SEQ ID NO: 479 - DNA, 90 bp, synthetic construct
  misc_feature 1..90: note = Description of Artificial Sequence: Synthetic oligonucleotide
  source 1..90: mol_type = other DNA
gtcttgtgttgccaagtctacctgtgccatcaacaacaccctcattgctttcttcattttgactacgataaaagacataaccagtgcggt

SEQ ID NO: 480 - DNA, 90 bp, synthetic construct
  misc_feature 1..90: note = Description of Artificial Sequence: Synthetic oligonucleotide
  source 1..90: mol_type = other DNA
ttttcttgtgttgccaagtctacctgtgccatcaacaacaccctcattgctttcttcattttgactacgataaaagacataaccagtgcg

SEQ ID NO: 481 - DNA, 90 bp, synthetic construct
  misc_feature 1..90: note = Description of Artificial Sequence: Synthetic oligonucleotide
  source 1..90: mol_type = other DNA
attttgtcttgtgttgccaagtctacctgtgccatcaacaacaccctcattgctttcttcattttgactacgataaaagacataaccagt

SEQ ID NO: 482 - DNA, 90 bp, synthetic construct
  misc_feature 1..90: note = Description of Artificial Sequence: Synthetic oligonucleotide
  source 1..90: mol_type = other DNA
acacgattttgtcttgtgttgccaagtctacctgtgccatcaacaacaccctcattgctttcttcattttgactacgataaaagacataa

SEQ ID NO: 483 - DNA, 166 bp, synthetic construct
  misc_feature 1..166: note = Description of Artificial Sequence: Synthetic polynucleotide
  source 1..166: mol_type = other DNA
atttagagcacagtgatgagcaagcagtaataaagtctcccttaaaatgcaccctccttccacctggccaggaatcagcattgggaatggtaccacctcccgaaaatgtcagaatgaattctgttaatttcaagaacattctacagtgggagtcacctgcttttgc

SEQ ID NO: 484 - protein, 55 aa, synthetic construct
  REGION 1..55: note = Description of Artificial Sequence: Synthetic polypeptide
  VARIANT 55: note = MOD_RES - Any amino acid
LEHSDEQAVIKSPLKCTLLPPGQESALGMVPPPENVRMNSVNFKNILQWESPAFX

SEQ ID NO: 485 - DNA, 90 bp, synthetic construct
  misc_feature 1..90: note = Description of Artificial Sequence: Synthetic oligonucleotide
  source 1..90: mol_type = other DNA
agcattgggaatggtaccacctcccgaaaatgtcagaatgaattctgttaatttcaagaacattctacagtgggagtcacctgcttttgc

SEQ ID NO: 486 - DNA, 90 bp, synthetic construct
  misc_feature 1..90: note = Description of Artificial Sequence: Synthetic oligonucleotide
  source 1..90: mol_type = other DNA
gtcagcattgggaatggtaccacctcccgaaaatgtcagaatgaattctgttaatttcaagaacattctacagtgggagtcacctgcttt

SEQ ID NO: 487 - DNA, 90 bp, synthetic construct
  misc_feature 1..90: note = Description of Artificial Sequence: Synthetic oligonucleotide
  source 1..90: mol_type = other DNA
cagtaataaagtctcccttaaaatgcaccctccttccacctggccaggaatcagcattgggaatggtaccacctcccgaaaatgtcagaa

SEQ ID NO: 488 - DNA, 90 bp, synthetic construct
  misc_feature 1..90: note = Description of Artificial Sequence: Synthetic oligonucleotide
  source 1..90: mol_type = other DNA
gtgatgagcaagcagtaataaagtctcccttaaaatgcaccctccttccacctggccaggaatcagcattgggaatggtaccacctcccg

SEQ ID NO: 489 - DNA, 90 bp, synthetic construct
  misc_feature 1..90: note = Description of Artificial Sequence: Synthetic oligonucleotide
  source 1..90: mol_type = other DNA
acagtgatgagcaagcagtaataaagtctcccttaaaatgcaccctccttccacctggccaggaatcagcattgggaatggtaccaccaa

SEQ ID NO: 490 - DNA, 90 bp, synthetic construct
  misc_feature 1..90: note = Description of Artificial Sequence: Synthetic oligonucleotide
  source 1..90: mol_type = other DNA
atttagagcacagtgatgagcaagcagtaataaagtctcccttaaaatgcaccctccttccacctggccaggaatcagcattgggaatgg

SEQ ID NO: 491 - protein, 12 aa, synthetic construct
  REGION 1..12: note = Description of Artificial Sequence: Synthetic peptide
LQLANEEIAQVR

SEQ ID NO: 492 - protein, 10 aa, synthetic construct
  REGION 1..10: note = Description of Artificial Sequence: Synthetic peptide
DGTGLVPSER

SEQ ID NO: 493 - protein, 13 aa, synthetic construct
  REGION 1..13: note = Description of Artificial Sequence: Synthetic peptide
VGPDGTPYVTVLK

SEQ ID NO: 494 - protein, 7 aa, synthetic construct
  REGION 1..7: note = Description of Artificial Sequence: Synthetic peptide
VLVGPQR

SEQ ID NO: 495 - DNA, 23 bp, synthetic construct
  misc_feature 1..23: note = Description of Artificial Sequence: Synthetic primer
  source 1..23: mol_type = other DNA
cgtgaagatgctgaaagacgatg

SEQ ID NO: 496 - DNA, 21 bp, synthetic construct
  misc_feature 1..21: note = Description of Artificial Sequence: Synthetic primer
  source 1..21: mol_type = other DNA
aaacgcttgaagaggtcggag

SEQ ID NO: 497 - DNA, 21 bp, synthetic construct
  misc_feature 1..21: note = Description of Artificial Sequence: Synthetic primer
  source 1..21: mol_type = other DNA
tgcctgtggaggaacttttca

SEQ ID NO: 498 - DNA, 20 bp, synthetic construct
  misc_feature 1..20: note = Description of Artificial Sequence: Synthetic primer
  source 1..20: mol_type = other DNA
cccaaactcagcagcctaag

SEQ ID NO: 499 - DNA, 19 bp, synthetic construct
  misc_feature 1..19: note = Description of Artificial Sequence: Synthetic primer
  source 1..19: mol_type = other DNA
tggaccgtgtccttaccgt

SEQ ID NO: 500 - DNA, 19 bp, synthetic construct
  misc_feature 1..19: note = Description of Artificial Sequence: Synthetic primer
  source 1..19: mol_type = other DNA
ggtcctttggggtcctgct

SEQ ID NO: 501 - DNA, 20 bp, synthetic construct
  misc_feature 1..20: note = Description of Artificial Sequence: Synthetic primer
  source 1..20: mol_type = other DNA
cctctttcagctccaaggca

SEQ ID NO: 502 - DNA, 21 bp, synthetic construct
  misc_feature 1..21: note = Description of Artificial Sequence: Synthetic primer
  source 1..21: mol_type = other DNA
tctaccaggactgtccctcag

SEQ ID NO: 503 - DNA, 20 bp, synthetic construct
  misc_feature 1..20: note = Description of Artificial Sequence: Synthetic primer
  source 1..20: mol_type = other DNA
gggagtctcatttgcaccgt

SEQ ID NO: 504 - DNA, 20 bp, synthetic construct
  misc_feature 1..20: note = Description of Artificial Sequence: Synthetic primer
  source 1..20: mol_type = other DNA
ctgcatccaggtccttctgg

SEQ ID NO: 505 - DNA, 20 bp, synthetic construct
  misc_feature 1..20: note = Description of Artificial Sequence: Synthetic primer
  source 1..20: mol_type = other DNA
ccagttccaggttcttcccg

SEQ ID NO: 506 - DNA, 21 bp, synthetic construct
  misc_feature 1..21: note = Description of Artificial Sequence: Synthetic primer
  source 1..21: mol_type = other DNA
caacctcttcgaacctgtcca

SEQ ID NO: 507 - DNA, 20 bp, synthetic construct
  misc_feature 1..20: note = Description of Artificial Sequence: Synthetic primer
  source 1..20: mol_type = other DNA
aagacgatgccactgacaag

SEQ ID NO: 508 - DNA, 24 bp, synthetic construct
  misc_feature 1..24: note = Description of Artificial Sequence: Synthetic primer
  source 1..24: mol_type = other DNA
cccagcaggttgatgatgtttttg

SEQ ID NO: 509 - DNA, 22 bp, synthetic construct
  misc_feature 1..22: note = Description of Artificial Sequence: Synthetic primer
  source 1..22: mol_type = other DNA
tccttctccgacctcttcaagc

SEQ ID NO: 510 - DNA, 23 bp, synthetic construct
  misc_feature 1..23: note = Description of Artificial Sequence: Synthetic primer
  source 1..23: mol_type = other DNA
taatcctccacgcacttcttcag

SEQ ID NO: 511 - DNA, 18 bp, synthetic construct
  misc_feature 1..18: note = Description of Artificial Sequence: Synthetic primer
  source 1..18: mol_type = other DNA
tacctggacctgtcggcg

SEQ ID NO: 512 - DNA, 18 bp, synthetic construct
  misc_feature 1..18: note = Description of Artificial Sequence: Synthetic primer
  source 1..18: mol_type = other DNA
tgggcaaacacggagtcg

SEQ ID NO: 513 - DNA, 25 bp, synthetic construct
  misc_feature 1..25: note = Description of Artificial Sequence: Synthetic primer
  source 1..25: mol_type = other DNA
ccacagacgcacaggattctaagtc

SEQ ID NO: 514 - DNA, 22 bp, synthetic construct
  misc_feature 1..22: note = Description of Artificial Sequence: Synthetic primer
  source 1..22: mol_type = other DNA
tgagttttccagtccaagggtg

SEQ ID NO: 515 - DNA, 30 bp, synthetic construct
  misc_feature 1..30: note = Description of Artificial Sequence: Synthetic oligonucleotide
  source 1..30: mol_type = other DNA
gtgacgtccaccgactttaaggagtcggcc

SEQ ID NO: 516 - protein, 10 aa, synthetic construct
  REGION 1..10: note = Description of Artificial Sequence: Synthetic peptide
VTSTDFKESA

SEQ ID NO: 517 - DNA, 30 bp, synthetic construct
  misc_feature 1..30: note = Description of Artificial Sequence: Synthetic oligonucleotide
  source 1..30: mol_type = other DNA
gtgacgtccaccgacgtaaaggcgacacag

SEQ ID NO: 518 - protein, 10 aa, synthetic construct
  REGION 1..10: note = Description of Artificial Sequence: Synthetic peptide
VTSTDVKATQ

SEQ ID NO: 519 - DNA, 30 bp, synthetic construct
  misc_feature 1..30: note = Description of Artificial Sequence: Synthetic oligonucleotide
  source 1..30: mol_type = other DNA
gcgaagaggggctcggtggtgcagttggca

SEQ ID NO: 520 - DNA, 30 bp, synthetic construct
  misc_feature 1..30: note = Description of Artificial Sequence: Synthetic oligonucleotide
  source 1..30: mol_type = other DNA
cctcccgccagcaggagcccccagcagcct

SEQ ID NO: 521 - DNA, 30 bp, synthetic construct
  misc_feature 1..30: note = Description of Artificial Sequence: Synthetic oligonucleotide
  source 1..30: mol_type = other DNA
gactcagtgcagatgttccctcgccgcact

SEQ ID NO: 522 - DNA, 30 bp, synthetic construct
  misc_feature 1..30: note = Description of Artificial Sequence: Synthetic oligonucleotide
  source 1..30: mol_type = other DNA
gcctgggcacagagggtgtccgagagcagc

SEQ ID NO: 523 - DNA, 30 bp, synthetic construct
  misc_feature 1..30: note = Description of Artificial Sequence: Synthetic oligonucleotide
  source 1..30: mol_type = other DNA
ccacccgcctatgccaggcaattccgcgaa

SEQ ID NO: 524 - DNA, 30 bp, synthetic construct
  misc_feature 1..30: note = Description of Artificial Sequence: Synthetic oligonucleotide
  source 1..30: mol_type = other DNA
tggacctgtcggcgcaggagcaacggcagc

SEQ ID NO: 525 - DNA, 30 bp, synthetic construct
  misc_feature 1..30: note = Description of Artificial Sequence: Synthetic oligonucleotide
  source 1..30: mol_type = other DNA
gagtgctggctctggcccgggactctccgg

SEQ ID NO: 526 - DNA, 30 bp, synthetic construct
  misc_feature 1..30: note = Description of Artificial Sequence: Synthetic oligonucleotide
  source 1..30: mol_type = other DNA
aggtggctgtgcgaaggtcgctgagggtcc

SEQ ID NO: 527 - DNA, 31 bp, synthetic construct
  misc_feature 1..31: note = Description of Artificial Sequence: Synthetic oligonucleotide
  source 1..31: mol_type = other DNA
tgccgtccccggccatccctcaggacgtccg

SEQ ID NO: 528 - DNA, 2421 bp, Homo sapiens
  source 1..2421: mol_type = unassigned DNA
atgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacgaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacgagtacctggacctgtcggcgcctttcgagcagtactccccgggtggccaggacacccccagctccagctcctcaggggacgactccgtgtttgcccacgacctgctgcccccggccccacccagcagtgggggctcgcggacgtga

SEQ ID NO: 529 - DNA, 2517 bp, Homo sapiens
  source 1..2517: mol_type = unassigned DNA
atgagtctgcaggtcttaaacgacaaaaatgtcagcaatgaaaaaaatacagaaaattgcgacttcctgttttcgccaccagaagttaccggaagatcgtctgttcttcgtgtgtcacagaaagaaaatgtgccacccaagaacctggccaaagctatgaaggtgacttttcagacacctctgcgggatccacagacgcacaggattctaagtcctagcatggccagcaaacttgaggctcctttcactcaggatgacacccttggactggaaaactcacacccggtctggacacagaaagagaaccaacagctcatcaaggaagtggatgccaaaactactcatggaattctacagaaaccagtggaggctgacaccgacctcctgggggatgcaagcccagcctttgggagtggcagctccagcgagtctggcccaggtgccctggctgacctggactgctcaagctcttcccagagcccaggaagttctgagaaccaaatggtgtctccaggaaaagtgtctggcagccctgagcaagccgtggaggaaaaccttagttcctattccttagacagaagagtgacacccgcctctgagaccctagaagacccttgcaggacagagtcccagcacaaagcggagactccgcacggagccgaggaagaatgcaaagcggagactccgcacggagccgaggaggaatgccggcacggtggggtctgtgctcccgcagcagtggccacttcgcctcctggtgcaatccctaaggaagcctgcggaggagcacccctgcagggtctgcctggcgaagccctgggctgccctgcgggtgtgggcacccccgtgccagcagatggcactcagacccttacctgtgcacacacctctgctcctgagagcacagccccaaccaaccacctggtggctggcagggccatgaccctgagtcctcaggaagaagtggctgcaggccaaatggccagctcctcgaggagcggacctgtaaaactagaatttgatgtatctgatggcgccaccagcaaaagggcacccccaccaaggagactgggagagaggtccggcctcaagcctcccttgaggaaagcagcagtgaggcagcaaaaggccccgcaggaggtggaggaggacgacggtaggagcggagcaggagaggacccccccatgccagcttctcggggctcttaccacctcgactgggacaaaatggatgacccaaacttcatcccgttcggaggtgacaccaagtctggttgcagtgaggcccagcccccagaaagccctgagaccaggctgggccagccagcggctgaacagttgcatgctgggcctgccacggaggagccaggtccctgtctgagccagcagctgcattcagcctcagcggaggacacgcctgtggtgcagttggcagccgagaccccaacagcagagagcaaggagagagccttgaactctgccagcacctcgcttcccacaagctgtccaggcagtgagccagtgcccacccatcagcaggggcagcctgccttggagctgaaagaggagagcttcagagaccccgctgaggttctaggcacgggcgcggaggtggattacctggagcagtttggaacttcctcgtttaaggagtcggccttgaggaagcagtccttatacctcaagttcgaccccctcctgagggacagtcctggtagaccagtgcccgtggccaccgagaccagcagcatgcacggtgcaaatgagactccctcaggacgtccgcgggaagccaagcttgtggagttcgatttcttgggagcactggacattcctgtgccaggcccacccccaggtgttcccgcgcctgggggcccacccctgtccaccggacctatagtggacctgctccagtacagccagaaggacctggatgcagtggtaaaggcgacacaggaggagaaccgggagctgaggagcaggtgtgaggagctccacgggaagaacctggaactggggaagatcatggacaggttcgaagaggttgtgtaccaggccatggaggaagttcagaagcagaaggaactttccaaagctgaaatccagaaagttctaaaagaaaaagaccaacttaccacagatctgaactccatggagaagtccttctccgacctcttcaagcgttttgagaaacagaaagaggtgatcgagggctaccgcaagaacgaagagtcactgaagaagtgcgtggaggattacctggcaaggatcacccaggagggccagaggtaccaagccctgaaggcccacgcggaggagaagctgcagctggcaaacgaggagatcgcccaggtccggagcaaggcccaggcggaagcgttggccctccaggccagcctgaggaaggagcagatgcgcatccagtcgctggagaagacagtggagcagaagactaaagagaacgaggagctgaccaggatctgcgacgacctcatctccaagatggagaagatctga

SEQ ID NO: 530 - DNA, 2850 bp, synthetic construct
  misc_feature 1..2850: note = Description of Artificial Sequence: Synthetic polynucleotide
  source 1..2850: mol_type = other DNA
atgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacgaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacgtaaaggcgacacaggaggagaaccgggagctgaggagcaggtgtgaggagctccacgggaagaacctggaactggggaagatcatggacaggttcgaagaggttgtgtaccaggccatggaggaagttcagaagcagaaggaactttccaaagctgaaatccagaaagttctaaaagaaaaagaccaacttaccacagatctgaactccatggagaagtccttctccgacctcttcaagcgttttgagaaacagaaagaggtgatcgagggctaccgcaagaacgaagagtcactgaagaagtgcgtggaggattacctggcaaggatcacccaggagggccagaggtaccaagccctgaaggcccacgcggaggagaagctgcagctggcaaacgaggagatcgcccaggtccggagcaaggcccaggcggaagcgttggccctccaggccagcctgaggaaggagcagatgcgcatccagtcgctggagaagacagtggagcagaagactaaagagaacgaggagctgaccaggatctgcgacgacctcatctccaagatggagaagatctga

SEQ ID NO: 531 - DNA, 3147 bp, synthetic construct
  misc_feature 1..3147: note = Description of Artificial Sequence: Synthetic polynucleotide
  source 1..3147: mol_type = other DNA
atgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacgaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgactttaaggagtcggccttgaggaagcagtccttatacctcaagttcgaccccctcctgagggacagtcctggtagaccagtgcccgtggccaccgagaccagcagcatgcacggtgcaaatgagactccctcaggacgtccgcgggaagccaagcttgtggagttcgatttcttgggagcactggacattcctgtgccaggcccacccccaggtgttcccgcgcctgggggcccacccctgtccaccggacctatagtggacctgctccagtacagccagaaggacctggatgcagtggtaaaggcgacacaggaggagaaccgggagctgaggagcaggtgtgaggagctccacgggaagaacctggaactggggaagatcatggacaggttcgaagaggttgtgtaccaggccatggaggaagttcagaagcagaaggaactttccaaagctgaaatccagaaagttctaaaagaaaaagaccaacttaccacagatctgaactccatggagaagtccttctccgacctcttcaagcgttttgagaaacagaaagaggtgatcgagggctaccgcaagaacgaagagtcactgaagaagtgcgtggaggattacctggcaaggatcacccaggagggccagaggtaccaagccctgaaggcccacgcggaggagaagctgcagctggcaaacgaggagatcgcccaggtccggagcaaggcccaggcggaagcgttggccctccaggccagcctgaggaaggagcagatgcgcatccagtcgctggagaagacagtggagcagaagactaaagagaacgaggagctgaccaggatctgcgacgacctcatctccaagatggagaagatctga

SEQ ID NO: 532 - DNA, 2955 bp, synthetic construct
  misc_feature 1..2955: note = Description of Artificial Sequence: Synthetic polynucleotide
  source 1..2955: mol_type = other DNA
atgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacgaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacgtgccaggcccacccccaggtgttcccgcgcctgggggcccacccctgtccaccggacctatagtggacctgctccagtacagccagaaggacctggatgcagtggtaaaggcgacacaggaggagaaccgggagctgaggagcaggtgtgaggagctccacgggaagaacctggaactggggaagatcatggacaggttcgaagaggttgtgtaccaggccatggaggaagttcagaagcagaaggaactttccaaagctgaaatccagaaagttctaaaagaaaaagaccaacttaccacagatctgaactccatggagaagtccttctccgacctcttcaagcgttttgagaaacagaaagaggtgatcgagggctaccgcaagaacgaagagtcactgaagaagtgcgtggaggattacctggcaaggatcacccaggagggccagaggtaccaagccctgaaggcccacgcggaggagaagctgcagctggcaaacgaggagatcgcccaggtccggagcaaggcccaggcggaagcgttggccctccaggccagcctgaggaaggagcagatgcgcatccagtcgctggagaagacagtggagcagaagactaaagagaacgaggagctgaccaggatctgcgacgacctcatctccaagatggagaagatctga

SEQ ID NO: 533 - DNA, 3330 bp, synthetic construct
  misc_feature 1..3330: note = Description of Artificial Sequence: Synthetic polynucleotide
  source 1..3330: mol_type = other DNA
atgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacgaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacgagagagccttgaactctgccagcacctcgcttcccacaagctgtccaggcagtgagccagtgcccacccatcagcaggggcagcctgccttggagctgaaagaggagagcttcagagaccccgctgaggttctaggcacgggcgcggaggtggattacctggagcagtttggaacttcctcgtttaaggagtcggccttgaggaagcagtccttatacctcaagttcgaccccctcctgagggacagtcctggtagaccagtgcccgtggccaccgagaccagcagcatgcacggtgcaaatgagactccctcaggacgtccgcgggaagccaagcttgtggagttcgatttcttgggagcactggacattcctgtgccaggcccacccccaggtgttcccgcgcctgggggcccacccctgtccaccggacctatagtggacctgctccagtacagccagaaggacctggatgcagtggtaaaggcgacacaggaggagaaccgggagctgaggagcaggtgtgaggagctccacgggaagaacctggaactggggaagatcatggacaggttcgaagaggttgtgtaccaggccatggaggaagttcagaagcagaaggaactttccaaagctgaaatccagaaagttctaaaagaaaaagaccaacttaccacagatctgaactccatggagaagtccttctccgacctcttcaagcgttttgagaaacagaaagaggtgatcgagggctaccgcaagaacgaagagtcactgaagaagtgcgtggaggattacctggcaaggatcacccaggagggccagaggtaccaagccctgaaggcccacgcggaggagaagctgcagctggcaaacgaggagatcgcccaggtccggagcaaggcccaggcggaagcgttggccctccaggccagcctgaggaaggagcagatgcgcatccagtcgctggagaagacagtggagcagaagactaaagagaacgaggagctgaccaggatctgcgacgacctcatctccaagatggagaagatctga

SEQ ID NO: 534 - DNA, 2780 bp, synthetic construct
  misc_feature 1..2780: note = Description of Artificial Sequence: Synthetic polynucleotide
  source 1..2780: mol_type = other DNA
atgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacgaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacgagtacctggacctgtcggcgcctttcgagcagtactccccgggtggccaggacacccccagaagcagaaggaactttccaaagctgaaatccagaaagttctaaaagaaaaagaccaacttaccacagatctgaactccatggagaagtccttctccgacctcttcaagcgttttgagaaacagaaagaggtgatcgagggctaccgcaagaacgaagagtcactgaagaagtgcgtggaggattacctggcaaggatcacccaggagggccagaggtaccaagccctgaaggcccacgcggaggagaagctgcagctggcaaacgaggagatcgcccaggtccggagcaaggcccaggcggaagcgttggccctccaggccagcctgaggaaggagcagatgcgcatccagtcgctggagaagacagtggagcagaagactaaagagaacgaggagctgaccaggatctgcgacgacctcatctccaagatggagaagatctga

SEQ ID NO: 535 - DNA, 3135 bp, synthetic construct
  misc_feature 1..3135: note = Description of Artificial Sequence: Synthetic polynucleotide
  source 1..3135: mol_type = other DNA
atgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacgaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacgagtacctggacctgtcggcgcaggagcaacggcagcctaccctccagccacagggctgctgccttgctggttacagccaccgtttctctagcatgcacggtgcaaatgagactccctcaggacgtccgcgggaagccaagcttgtggagttcgatttcttgggagcactggacattcctgtgccaggcccacccccaggtgttcccgcgcctgggggcccacccctgtccaccggacctatagtggacctgctccagtacagccagaaggacctggatgcagtggtaaaggcgacacaggaggagaaccgggagctgaggagcaggtgtgaggagctccacgggaagaacctggaactggggaagatcatggacaggttcgaagaggttgtgtaccaggccatggaggaagttcagaagcagaaggaactttccaaagctgaaatccagaaagttctaaaagaaaaagaccaacttaccacagatctgaactccatggagaagtccttctccgacctcttcaagcgttttgagaaacagaaagaggtgatcgagggctaccgcaagaacgaagagtcactgaagaagtgcgtggaggattacctggcaaggatcacccaggagggccagaggtaccaagccctgaaggcccacgcggaggagaagctgcagctggcaaacgaggagatcgcccaggtccggagcaaggcccaggcggaagcgttggccctccaggccagcctgaggaaggagcagatgcgcatccagtcgctggagaagacagtggagcagaagactaaagagaacgaggagctgaccaggatctgcgacgacctcatctccaagatggagaagatctga

SEQ ID NO: 536 - DNA, 3396 bp, synthetic construct
  misc_feature 1..3396: note = Description of Artificial Sequence: Synthetic polynucleotide
  source 1..3396: mol_type = other DNA
atgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacgaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacgagtacctggacctgtcggcgcctgtggtgcagttggcagccgagaccccaacagcagagagcaaggagagagccttgaactctgccagcacctcgcttcccacaagctgtccaggcagtgagccagtgcccacccatcagcaggggcagcctgccttggagctgaaagaggagagcttcagagaccccgctgaggttctaggcacgggcgcggaggtggattacctggagcagtttggaacttcctcgtttaaggagtcggccttgaggaagcagtccttatacctcaagttcgaccccctcctgagggacagtcctggtagaccagtgcccgtggccaccgagaccagcagcatgcacggtgcaaatgagactccctcaggacgtccgcgggaagccaagcttgtggagttcgatttcttgggagcactggacattcctgtgccaggcccacccccaggtgttcccgcgcctgggggcccacccctgtccaccggacctatagtggacctgctccagtacagccagaaggacctggatgcagtggtaaaggcgacacaggaggagaaccgggagctgaggagcaggtgtgaggagctccacgggaagaacctggaactggggaagatcatggacaggttcgaagaggttgtgtaccaggccatggaggaagttcagaagcagaaggaactttccaaagctgaaatccagaaagttctaaaagaaaaagaccaacttaccacagatctgaactccatggagaagtccttctccgacctcttcaagcgttttgagaaacagaaagaggtgatcgagggctaccgcaagaacgaagagtcactgaagaagtgcgtggaggattacctggcaaggatcacccaggagggccagaggtaccaagccctgaaggcccacgcggaggagaagctgcagctggcaaacgaggagatcgcccaggtccggagcaaggcccaggcggaagcgttggccctccaggccagcctgaggaaggagcagatgcgcatccagtcgctggagaagacagtggagcagaagactaaagagaacgaggagctgaccaggatctgcgacgacctcatctccaagatggagaagatctga

SEQ ID NO: 537 - DNA, 3504 bp, synthetic construct
  misc_feature 1..3504: note = Description of Artificial Sequence: Synthetic polynucleotide
  source 1..3504: mol_type = other DNA
atgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacgaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacgagtacctggacctgtcggcgcctttcgagcagtactccccgggtggccaggacacccccagctccagctcctcaggggacgtgcgtgagccaccgcacccggcgtttcctaggtcagcggaggacacgcctgtggtgcagttggcagccgagaccccaacagcagagagcaaggagagagccttgaactctgccagcacctcgcttcccacaagctgtccaggcagtgagccagtgcccacccatcagcaggggcagcctgccttggagctgaaagaggagagcttcagagaccccgctgaggttctaggcacgggcgcggaggtggattacctggagcagtttggaacttcctcgtttaaggagtcggccttgaggaagcagtccttatacctcaagttcgaccccctcctgagggacagtcctggtagaccagtgcccgtggccaccgagaccagcagcatgcacggtgcaaatgagactccctcaggacgtccgcgggaagccaagcttgtggagttcgatttcttgggagcactggacattcctgtgccaggcccacccccaggtgttcccgcgcctgggggcccacccctgtccaccggacctatagtggacctgctccagtacagccagaaggacctggatgcagtggtaaaggcgacacaggaggagaaccgggagctgaggagcaggtgtgaggagctccacgggaagaacctggaactggggaagatcatggacaggttcgaagaggttgtgtaccaggccatggaggaagttcagaagcagaaggaactttccaaagctgaaatccagaaagttctaaaagaaaaagaccaacttaccacagatctgaactccatggagaagtccttctccgacctcttcaagcgttttgagaaacagaaagaggtgatcgagggctaccgcaagaacgaagagtcactgaagaagtgcgtggaggattacctggcaaggatcacccaggagggccagaggtaccaagccctgaaggcccacgcggaggagaagctgcagctggcaaacgaggagatcgcccaggtccggagcaaggcccaggcggaagcgttggccctccaggccagcctgaggaaggagcagatgcgcatccagtcgctggagaagacagtggagcagaagactaaagagaacgaggagctgaccaggatctgcgacgacctcatctccaagatggagaagatctga

SEQ ID NO: 538 - DNA, 3555 bp, synthetic construct
  misc_feature 1..3555: note = Description of Artificial Sequence: Synthetic polynucleotide
  source 1..3555: mol_type = other DNA
atgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacgaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacgagtacctggacctgtcggcgcctttcgagcagtactccccgggtggccaggacaccccagaaagccctgagaccaggctgggccagccagcggctgaacagttgcatgctgggcctgccacggaggagccaggtccctgtctgagccagcagctgcattcagcctcagcggaggacacgcctgtggtgcagttggcagccgagaccccaacagcagagagcaaggagagagccttgaactctgccagcacctcgcttcccacaagctgtccaggcagtgagccagtgcccacccatcagcaggggcagcctgccttggagctgaaagaggagagcttcagagaccccgctgaggttctaggcacgggcgcggaggtggattacctggagcagtttggaacttcctcgtttaaggagtcggccttgaggaagcagtccttatacctcaagttcgaccccctcctgagggacagtcctggtagaccagtgcccgtggccaccgagaccagcagcatgcacggtgcaaatgagactccctcaggacgtccgcgggaagccaagcttgtggagttcgatttcttgggagcactggacattcctgtgccaggcccacccccaggtgttcccgcgcctgggggcccacccctgtccaccggacctatagtggacctgctccagtacagccagaaggacctggatgcagtggtaaaggcgacacaggaggagaaccgggagctgaggagcaggtgtgaggagctccacgggaagaacctggaactggggaagatcatggacaggttcgaagaggttgtgtaccaggccatggaggaagttcagaagcagaaggaactttccaaagctgaaatccagaaagttctaaaagaaaaagaccaacttaccacagatctgaactccatggagaagtccttctccgacctcttcaagcgttttgagaaacagaaagaggtgatcgagggctaccgcaagaacgaagagtcactgaagaagtgcgtggaggattacctggcaaggatcacccaggagggccagaggtaccaagccctgaaggcccacgcggaggagaagctgcagctggcaaacgaggagatcgcccaggtccggagcaaggcccaggcggaagcgttggccctccaggccagcctgaggaaggagcagatgcgcatccagtcgctggagaagacagtggagcagaagactaaagagaacgaggagctgaccaggatctgcgacgacctcatctccaagatggagaagatctga

SEQ ID NO: 539 - protein, 949 aa, synthetic construct; identical to SEQ ID NO: 161
  REGION 1..949: note = Description of Artificial Sequence: Synthetic polypeptide

SEQ ID NO: 540 - protein, 1048 aa, synthetic construct; identical to SEQ ID NO: 79
  REGION 1..1048: note = Description of Artificial Sequence: Synthetic polypeptide

SEQ ID NO: 541 - protein, 984 aa, synthetic construct; identical to SEQ ID NO: 160
  REGION 1..984: note = Description of Artificial Sequence: Synthetic polypeptide

SEQ ID NO: 542 - protein, 1109 aa, synthetic construct
  REGION 1..1109: note = Description of Artificial Sequence: Synthetic polypeptide
MGAPACALALCVAVAIVAGASSESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAGILSYGVGFFLFILVVAAVTLCRLRSPPKKGLGSPTVHKISRFPLKRQVSLESNASMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREFLRARRPPGLDYSFDTCKPPEEQLTFKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAAPSQRPTFKQLVEDLDRVLTVTSTDERALNSASTSLPTSCPGSEPVPTHQQGQPALELKEESFRDPAEVLGTGAEVDYLEQFGTSSFKESALRKQSLYLKFDPLLRDSPGRPVPVATETSSMHGANETPSGRPREAKLVEFDFLGALDIPVPGPPPGVPAPGGPPLSTGPIVDLLQYSQKDLDAVVKATQEENRELRSRCEELHGKNLELGKIMDRFEEVVYQAMEEVQKQKELSKAEIQKVLKEKDQLTTDLNSMEKSFSDLFKRFEKQKEVIEGYRKNEESLKKCVEDYLARITQEGQRYQALKAHAEEKLQLANEEIAQVRSKAQAEALALQASLRKEQMRIQSLEKTVEQKTKENEELTRICDDLISKMEKI

SEQ ID NO: 543 - protein, 926 aa, synthetic construct
  REGION 1..926: note = Description of Artificial Sequence: Synthetic polypeptide
MGAPACALALCVAVAIVAGASSESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAGILSYGVGFFLFILVVAAVTLCRLRSPPKKGLGSPTVHKISRFPLKRQVSLESNASMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREFLRARRPPGLDYSFDTCKPPEEQLTFKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAAPSQRPTFKQLVEDLDRVLTVTSTDEYLDLSAPFEQYSPGGQDTPSKQKELSKAEIQKVLKEKDQLTTDLNSMEKSFSDLFKRFEKQKEVIEGYRKNEESLKKCVEDYLARITQEGQRYQALKAHAEEKLQLANEEIAQVRSKAQAEALALQASLRKEQMRIQSLEKTVEQKTKENEELTRICDDLISKMEKI

SEQ ID NO: 544 - protein, 1043 aa, synthetic construct
  REGION 1..1043: note = Description of Artificial Sequence: Synthetic polypeptide
MGAPACALALCVAVAIVAGASSESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAGILSYGVGFFLFILVVAAVTLCRLRSPPKKGLGSPTVHKISRFPLKRQVSLESNASMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREFLRARRPPGLDYSFDTCKPPEEQLTFKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAAPSQRPTFKQLVEDLDRVLTVTSTDEYLDLSAQEQRQPTLQPQGCCLAGYSHRSSMHGANETPSGRPREAKLVEFDFLGALDIPVPGPPPGVPAPGGPPLSTGPIVDLLQYSQKDLDAVVKATQEENRELRSRCEELHGKNLELGKIMDRFEEVVYQAMEEVQKQKELSKAEIQKVLKEKDQLTTDLNSMEKSFSDLFKRFEKQKEVIEGYRKNEESLKKCVEDYLARITQEGQRYQALKAHAEEKLQLANEEIAQVRSKAQAEALALQASLRKEQMRIQSLEKTVEQKTKENEELTRICDDLISKMEKI

SEQ ID NO: 545 - protein, 1131 aa, synthetic construct
  REGION 1..1131: note = Description of Artificial Sequence: Synthetic polypeptide
MGAPACALALCVAVAIVAGASSESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAGILSYGVGFFLFILVVAAVTLCRLRSPPKKGLGSPTVHKISRFPLKRQVSLESNASMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREFLRARRPPGLDYSFDTCKPPEEQLTFKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAAPSQRPTFKQLVEDLDRVLTVTSTDEYLDLSAPVVQLAAETPTAESKERALNSASTSLPTSCPGSEPVPTHQQGQPALELKEESFRDPAEVLGTGAEVDYLEQFGTSSFKESALRKQSLYLKFDPLLRDSPGRPVPVATETSSMHGANETPSGRPREAKLVEFDFLGALDIPVPGPPPGVPAPGGPPLSTGPIVDLLQYSQKDLDAVVKATQEENRELRSRCEELHGKNLELGKIMDRFEEVVYQAMEEVQKQKELSKAEIQKVLKEKDQLTTDLNSMEKSFSDLFKRFEKQKEVIEGYRKNEESLKKCVEDYLARITQEGQRYQALKAHAEEKLQLANEEIAQVRSKAQAEALALQASLRKEQMRIQSLEKTVEQKTKENEELTRICDDLISKMEKI

SEQ ID NO: 546 - protein, 1166 aa, synthetic construct
  REGION 1..1166: note = Description of Artificial Sequence: Synthetic polypeptide
  VARIANT 795: note = MOD_RES - Any amino acid
MGAPACALALCVAVAIVAGASSESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAGILSYGVGFFLFILVVAAVTLCRLRSPPKKGLGSPTVHKISRFPLKRQVSLESNASMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREFLRARRPPGLDYSFDTCKPPEEQLTFKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAAPSQRPTFKQLVEDLDRVLTVTSTDEYLDLSAPFEQYSPGGQDTPSSSSSGVREPPHPAFPXSAEDTPVVQLAAETPTAESKERALNSASTSLPTSCPGSEPVPTHQQGQPALELKEESFRDPAEVLGTGAEVDYLEQFGTSSFKESALRKQSLYLKFDPLLRDSPGRPVPVATETSSMHGANETPSGRPREAKLVEFDFLGALDIPVPGPPPGVPAPGGPPLSTGPIVDLLQYSQKDLDAVVKATQEENRELRSRCEELHGKNLELGKIMDRFEEVVYQAMEEVQKQKELSKAEIQKVLKEKDQLTTDLNSMEKSFSDLFKRFEKQKEVIEGYRKNEESLKKCVEDYLARITQEGQRYQALKAHAEEKLQLANEEIAQVRSKAQAEALALQASLRKEQMRIQSLEKTVEQKTKENEELTRICDDLISKMEKI

SEQ ID NO: 547 - protein, 1184 aa, synthetic construct
  REGION 1..1184: note = Description of Artificial Sequence: Synthetic polypeptide
MGAPACALALCVAVAIVAGASSESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAGILSYGVGFFLFILVVAAVTLCRLRSPPKKGLGSPTVHKISRFPLKRQVSLESNASMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREFLRARRPPGLDYSFDTCKPPEEQLTFKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAAPSQRPTFKQLVEDLDRVLTVTSTDEYLDLSAPFEQYSPGGQDTPESPETRLGQPAAEQLHAGPATEEPGPCLSQQLHSASAEDTPVVQLAAETPTAESKERALNSASTSLPTSCPGSEPVPTHQQGQPALELKEESFRDPAEVLGTGAEVDYLEQFGTSSFKESALRKQSLYLKFDPLLRDSPGRPVPVATETSSMHGANETPSGRPREAKLVEFDFLGALDIPVPGPPPGVPAPGGPPLSTGPIVDLLQYSQKDLDAVVKATQEENRELRSRCEELHGKNLELGKIMDRFEEVVYQAMEEVQKQKELSKAEIQKVLKEKDQLTTDLNSMEKSFSDLFKRFEKQKEVIEGYRKNEESLKKCVEDYLARITQEGQRYQALKAHAEEKLQLANEEIAQVRSKAQAEALALQASLRKEQMRIQSLEKTVEQKTKENEELTRICDDLISKMEKI

SEQ ID NO: 548 - DNA, 22 bp, synthetic construct
  misc_feature 1..22: note = Description of Artificial Sequence: Synthetic primer
  source 1..22: mol_type = other DNA
cttcggggagcagcgatgcgac

SEQ ID NO: 549 - DNA, 22 bp, synthetic construct
  misc_feature 1..22: note = Description of Artificial Sequence: Synthetic primer
  source 1..22: mol_type = other DNA
ctgtccatccagaggaggagta

SEQ ID NO: 550 - DNA, 32 bp, synthetic construct
  misc_feature 1..32: note = Description of Artificial Sequence: Synthetic oligonucleotide
  source 1..32: mol_type = other DNA
cgtgacgtccaccgactttaaggagtcggcct

SEQ ID NO: 551 - DNA, 32 bp, synthetic construct
  misc_feature 1..32: note = Description of Artificial Sequence: Synthetic oligonucleotide
  source 1..32: mol_type = other DNA
cgtgacgtccaccgacgtaaaggcgacacagg

SEQ ID NO: 552 - DNA, 32 bp, synthetic construct
  misc_feature 1..32: note = Description of Artificial Sequence: Synthetic oligonucleotide
  source 1..32: mol_type = other DNA
tgcgaagaggggctcggtggtgcagttggcag

SEQ ID NO: 553 - DNA, 32 bp, synthetic construct
  misc_feature 1..32: note = Description of Artificial Sequence: Synthetic oligonucleotide
  source 1..32: mol_type = other DNA
gcctcccgccagcaggagcccccagcagcctt

SEQ ID NO: 554 - DNA, 32 bp, synthetic construct
  misc_feature 1..32: note = Description of Artificial Sequence: Synthetic oligonucleotide
  source 1..32: mol_type = other DNA
agactcagtgcagatgttccctcgccgcactg

SEQ ID NO: 555 - DNA, 32 bp, synthetic construct
  misc_feature 1..32: note = Description of Artificial Sequence: Synthetic oligonucleotide
  source 1..32: mol_type = other DNA
agcctgggcacagagggtgtccgagagcagcc

SEQ ID NO: 556 - DNA, 32 bp, synthetic construct
  misc_feature 1..32: note = Description of Artificial Sequence: Synthetic oligonucleotide
  source 1..32: mol_type = other DNA
gccacccgcctatgccaggcaattccgcgaag

SEQ ID NO: 557 - DNA, 32 bp, synthetic construct
  misc_feature 1..32: note = Description of Artificial Sequence: Synthetic oligonucleotide
  source 1..32: mol_type = other DNA
ctggacctgtcggcgcaggagcaacggcagcc

SEQ ID NO: 558 - DNA, 32 bp, synthetic construct
  misc_feature 1..32: note = Description of Artificial Sequence: Synthetic oligonucleotide
  source 1..32: mol_type = other DNA
gagtgctggctctggcccgggactctccggcc

SEQ ID NO: 559 - DNA, 32 bp, synthetic construct
  misc_feature 1..32: note = Description of Artificial Sequence: Synthetic oligonucleotide
  source 1..32: mol_type = other DNA
gaggtggctgtgcgaaggtcgctgagggtcca

SEQ ID NO: 560 - DNA, 33 bp, synthetic construct
  misc_feature 1..33: note = Description of Artificial Sequence: Synthetic oligonucleotide
  source 1..33: mol_type = other DNA
ctgccgtccccggccatccctcaggacgtccgc

(19) United States
(12) Patent Application Publication
(10) Pub. No.: US 2024/0247275 A1
(43) Pub. Date: Jul. 25, 2024

(54) FUSION PROTEINS AND METHODS THEREOF

(71) Applicant: The Trustees of Columbia University in the City of New York, New York, NY (US)

(72) Inventors: Antonio IAVARONE, Sunny Isles Beach, FL (US); Anna LASORELLA, New York, NY (US); Raul RABADAN, New York, NY (US)

(21) Appl. No.: 18/416,643

(22) Filed: Jan. 18, 2024

Related U.S. Application Data
(62) Division of application No. 16/507,977, filed on Jul. 10, 2019, now Pat. No. 11,920,138.
Application No. 16/507,977 is a continuation of application No. 14/604,530, filed on Jan. 23, 2015, now abandoned.
Application No. 14/604,530 is a continuation-in-part of application No. PCT/US2013/051888, filed on Jul. 24, 2013.
(60) Provisional application No. 62/096,311, filed on Dec. 23, 2014.
(60) Provisional application No. 61/675,006, filed on Jul. 24, 2012.

(51) Int. Cl. C12N 15/62; C07K 14/47; C07K 14/71; C07K 14/82; C07K 16/18; C07K 16/28; C12N 9/12; C12Q 1/6886; G01N 33/574

(52) U.S. Cl. CPC C12N 15/62; C07K 14/47; C07K 14/71; C07K 14/82; C07K 16/18; C07K 16/2863; C12N 9/12; C12Q 1/6886; G01N 33/57492; C07K 2319/00; C07K 2319/73; C12Q 2600/158; G01N 2333/91205

(57) ABSTRACT

The invention discloses oncogenic fusion proteins. The invention provides methods for treating gene-fusion based cancers.

This application is a divisional of U.S. patent application Ser. No. 16/507,977, filed Jul. 10, 2019, which is a continuation of U.S. patent application Ser. No. 14/604,530, filed Jan. 23, 2015, which is a continuation-in-part of International Application No. PCT/US2013/051888, filed on Jul. 24, 2013, which claims priority to U.S. Provisional Patent Application No. 61/675,006, filed on Jul. 24, 2012, the content of each of which is hereby incorporated by reference in their entireties. U.S. patent application Ser. No. 14/604,530, filed Jan. 23, 2015, also claims priority to U.S. Provisional Patent Application No. 62/096,311, filed on Dec. 23, 2014, the content of which is hereby incorporated by reference in its entirety.

### GOVERNMENT SUPPORT

This invention was made with government support under grant numbers CA101644, CA131126, CA178546, and NS061776 awarded by the National Institutes of Health. The Government has certain rights in the invention.

All patents, patent applications and publications cited herein are hereby incorporated by reference in their entirety. The disclosures of these publications in their entireties are hereby incorporated by reference into this application.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

The instant application contains a Sequence Listing which has been submitted electronically in XML file format and is hereby incorporated by reference in its entirety. Said XML copy, created on Jan. 5, 2024, is named 0019240_00984US8_SL.xml and is 846,151 bytes in size.

### BACKGROUND OF THE INVENTION

Glioblastoma multiforme (GBM) is the most common form of brain cancer and among the most incurable and lethal of all human cancers. The current standard of care includes surgery, chemotherapy, and radiation therapy. However, the prognosis of GBM remains uniformly poor. There are few available targeted therapies and none that specifically target GBM.

The target population of GBM patients who may carry FGFR-TACC gene fusions and would benefit from targeted inhibition of FGFR kinase activity is estimated to correspond to 6,000 patients per year world-wide.

### SUMMARY OF THE INVENTION

The invention is based, at least in part, on the discovery of a highly expressed class of gene fusions in GBM, which join the tyrosine kinase domain of FGFR genes to the TACC domain of TACC1 or TACC3. The invention is based, at least in part, on the finding that FGFR-TACC fusions identify a subset of GBM patients who will benefit from targeted inhibition of the tyrosine kinase activity of FGFR. Identification of fusions of FGFR and TACC genes in glioblastoma patients and other subjects afflicted with a gene-fusion associated cancer (such as an epithelial cancer) are useful therapeutic targets.

The invention is also based, at least in part, on the discovery of gene fusions joining the tyrosine kinase domain of FGFR genes to the TACC domain of TACC1 or TACC3 in grade II and III glioma, The invention is based, at least in part, on the finding that FGFR-TACC fusions identify a subset of grade II and III glioma patients who will benefit from targeted inhibition of the tyrosine kinase activity of FGFR. Identification of fusions of FGFR and TACC genes in glioma patients are useful therapeutic targets.

An aspect of the invention provides for a purified fusion protein comprising a tyrosine kinase domain of an FGFR protein fused to a polypeptide that constitutively activates the tyrosine kinase domain of the FGFR protein. In one embodiment, the FGFR protein is FGFR1, FGFR2, FGFR3, or FGR4. In another embodiment, the purified fusion protein is essentially free of other human proteins.

An aspect of the invention provides for a purified fusion protein comprising a transforming acidic coiled-coil (TACC) domain fused to a polypeptide with a tyrosine kinase domain, wherein the TACC domain constitutively activates the tyrosine kinase domain. In one embodiment, the TACC protein is TACC1, TACC2, or TACC3. In another embodiment, the purified fusion protein is essentially free of other human proteins.

An aspect of the invention provides for a purified fusion protein comprising the tyrosine kinase domain of an FGFR protein fused 5′ to the TACC domain of a transforming acidic coiled-coil-containing (TACC) protein. In one embodiment, the FGFR protein is FGFR1, FGFR2, FGFR3, or FGR4. In another embodiment, the TACC protein is TACC1, TACC2, or TACC3. In another embodiment, the purified fusion protein is essentially free of other human proteins.

An aspect of the invention provides for a purified fusion protein encoded by an FGFR1-TACC1 nucleic acid, wherein FGFR1-TACC1 comprises a combination of exons 1-17 of FGFR1 located on human chromosome 8p11 spliced 5′ to a combination of exons 7-13 of TACC1 located on human chromosome 8p11, wherein a genomic breakpoint occurs in any one of exons 1-17 of FGFR1 and any one of exons 7-13 of TACC1. In another embodiment, the purified fusion protein is essentially free of other human proteins.

An aspect of the invention provides for a purified fusion protein encoded by an FGFR2-TACC2 nucleic acid, wherein FGFR2-TACC2 comprises a combination of any exons 1-18 of FGFR2 located on human chromosome 10q26 spliced 5′ to a combination of any exons 1-23 of TACC2 located on human chromosome 10q26. In another embodiment, the purified fusion protein is essentially free of other human proteins.

An aspect of the invention provides for a purified fusion protein encoded by an FGFR3-TACC3 nucleic acid, wherein FGFR3-TACC3 comprises a combination of exons 1-16 of FGFR3 located on human chromosome 4p16 spliced 5′ to a combination of exons 8-16 of TACC3 located on human chromosome 4p16, wherein a genomic breakpoint occurs in any one of exons 1-16 of FGFR3 and any one of exons 8-16 of TACC3. In another embodiment, the purified fusion protein is essentially free of other human proteins.

An aspect of the invention provides for a purified fusion protein encoded by an FGFR3-TACC3 nucleic acid, wherein FGFR3-TACC3 comprises a combination of exons 1-18 of FGFR3 located on human chromosome 4p16 spliced 5′ to a combination of exons 4-16 of TACC3 located on human chromosome 4p16, wherein a genomic breakpoint occurs in any one of exons 1-18 of FGFR3 and any one of exons 4-16 of TACC3. In another embodiment, the purified fusion protein is essentially free of other human proteins.

An aspect of the invention provides for a purified fusion protein encoded by an FGFR3-TACC3 nucleic acid, wherein FGFR3-TACC3 comprises a combination of exons 1-16 of FGFR3 located on human chromosome 4p16 spliced 5′ to a combination of exons 8-16 of TACC3 located on human chromosome 4p16, wherein a genomic breakpoint occurs in any one of introns 1-16 of FGFR3 and any one of exons 8-16 of TACC3. In another embodiment, the purified fusion protein is essentially free of other human proteins.

An aspect of the invention provides for a purified fusion protein encoded by an FGFR3-TACC3 nucleic acid, wherein FGFR3-TACC3 comprises a combination of exons 1-18 of FGFR3 located on human chromosome 4p16 spliced 5′ to a combination of exons 4-16 of TACC3 located on human chromosome 4p16, wherein a genomic breakpoint occurs in any one of introns 1-18 of FGFR3 and any one of exons 4-16 of TACC3. In another embodiment, the purified fusion protein is essentially free of other human proteins.

An aspect of the invention provides for a purified fusion protein encoded by an FGFR3-TACC3 nucleic acid, wherein FGFR3-TACC3 comprises a combination of exons 1-16 of FGFR3 located on human chromosome 4p16 spliced 5′ to a combination of exons 8-16 of TACC3 located on human chromosome 4p16, wherein a genomic breakpoint occurs in any one of exons 1-16 of FGFR3 and any one of introns 7-16 of TACC3. In another embodiment, the purified fusion protein is essentially free of other human proteins.

An aspect of the invention provides for a purified fusion protein encoded by an FGFR3-TACC3 nucleic acid, wherein FGFR3-TACC3 comprises a combination of exons 1-18 of FGFR3 located on human chromosome 4p16 spliced 5′ to a combination of exons 4-16 of TACC3 located on human chromosome 4p16, wherein a genomic breakpoint occurs in any one of exons 1-18 of FGFR3 and any one of introns 3-16 of TACC3. In another embodiment, the purified fusion protein is essentially free of other human proteins.

An aspect of the invention provides for a purified fusion protein encoded by an FGFR3-TACC3 nucleic acid, wherein FGFR3-TACC3 comprises a combination of exons 1-16 of FGFR3 located on human chromosome 4p16 spliced 5′ to a combination of exons 8-16 of TACC3 located on human chromosome 4p16, wherein a genomic breakpoint occurs in any one of introns 1-16 of FGFR3 and any one of introns 7-16 of TACC3. In another embodiment, the purified fusion protein is essentially free of other human proteins.

An aspect of the invention provides for a purified fusion protein encoded by an FGFR3-TACC3 nucleic acid, wherein FGFR3-TACC3 comprises a combination of exons 1-18 of FGFR3 located on human chromosome 4p16 spliced 5′ to a combination of exons 4-16 of TACC3 located on human chromosome 4p16, wherein a genomic breakpoint occurs in any one of introns 1-18 of FGFR3 and any one of introns 3-16 of TACC3. In another embodiment, the purified fusion protein is essentially free of other human proteins.

An aspect of the invention provides for a synthetic nucleic acid encoding the fusion proteins described above.

An aspect of the invention provides for a purified FGFR3-TACC3 fusion protein comprising SEQ ID NO: 79, 158, 159, 160, 161, 539, 540, 541, 542, 543, 544, 545, 546, 547. In another embodiment, the purified fusion protein is essentially free of other human proteins.

An aspect of the invention provides for a purified FGFR3-TACC3 fusion protein having a genomic breakpoint comprising at least 3 consecutive amino acids from amino acids 730-758 of SEQ ID NO: 90 and comprising at least 3 consecutive amino acids from amino acids 549-838 of SEQ ID NO: 92. In another embodiment, the purified fusion protein is essentially free of other human proteins.

An aspect of the invention provides for a purified FGFR3-TACC3 fusion protein having a genomic breakpoint comprising at least 3 consecutive amino acids from amino acids 730-781 of SEQ ID NO: 90 and comprising at least 3 consecutive amino acids from amino acids 432-838 of SEQ ID NO: 92. In another embodiment, the purified fusion protein is essentially free of other human proteins.

An aspect of the invention provides for a purified FGFR3-TACC3 fusion protein having a genomic breakpoint comprising SEQ ID NO: 78. In another embodiment, the purified fusion protein is essentially free of other human proteins.

An aspect of the invention provides for a purified FGFR3-TACC3 fusion protein having a genomic breakpoint comprising any one of SEQ ID NOS: 85, 86, 87, 89, 516 or 518. In another embodiment, the purified fusion protein is essentially free of other human proteins.

An aspect of the invention provides for a purified FGFR1-TACC1 fusion protein comprising SEQ ID NO: 150. In another embodiment, the purified fusion protein is essentially free of other human proteins.

An aspect of the invention provides for a purified FGFR1-TACC1 fusion protein having a genomic breakpoint comprising at least 3 consecutive amino acids from amino acids 746-762 of SEQ ID NO: 146 and comprising at least 3 consecutive amino acids from amino acids 572-590 of SEQ ID NO: 148. In another embodiment, the purified fusion protein is essentially free of other human proteins.

An aspect of the invention provides for a purified FGFR1-TACC1 fusion protein having a genomic breakpoint comprising at least 3 consecutive amino acids from amino acids 746-762 of SEQ ID NO: 146 and comprising at least 3 consecutive amino acids from amino acids 571-590 of SEQ ID NO: 148. In another embodiment, the purified fusion protein is essentially free of other human proteins.

An aspect of the invention provides for a purified FGFR1-TACC1 fusion protein having a genomic breakpoint comprising SEQ ID NO: 88. In another embodiment, the purified fusion protein is essentially free of other human proteins.

An aspect of the invention provides for a purified DNA encoding an FGFR3-TACC3 fusion protein comprising SEQ ID NO: 94, 530, 531, 532, 533, 534, 535, 536, 537, or 538. In another embodiment, the purified fusion protein is essentially free of other human proteins. An aspect of the invention provides for a purified cDNA encoding an FGFR3-TACC3 fusion protein comprising SEQ ID NO: 94, 530, 531, 532, 533, 534, 535, 536, 537, or 538.

An aspect of the invention provides for a synthetic nucleic acid encoding an FGFR3-TACC3 fusion protein having a genomic breakpoint comprising at least 9 consecutive in-frame nucleotides from nucleotides 2443-2530 of SEQ ID NO: 91 and comprising at least 9 consecutive in-frame nucleotides from nucleotides 1800-2847 of SEQ ID NO: 93.

An aspect of the invention provides for a synthetic nucleic acid encoding an FGFR3-TACC3 fusion protein having a genomic breakpoint comprising any one of SEQ ID NOS: 1-77, or 519-527.

An aspect of the invention provides for a synthetic nucleic acid encoding an FGFR1-TACC1 fusion protein comprising SEQ ID NO: 151.

An aspect of the invention provides for a synthetic nucleic acid encoding an FGFR1-TACC1 fusion protein having a genomic breakpoint comprising at least 9 consecutive in-frame nucleotides from nucleotides 3178-3228 of SEQ ID NO: 147 and comprising at least 9 consecutive in-frame nucleotides from nucleotides 2092-2794 of SEQ ID NO: 149.

An aspect of the invention provides for a synthetic nucleic acid encoding an FGFR1-TACC1 fusion protein having a genomic breakpoint comprising SEQ ID NO: 83.

An aspect of the invention provides for an antibody or antigen-binding fragment thereof, that specifically binds to a purified fusion protein comprising a tyrosine kinase domain of an FGFR protein fused to a polypeptide that constitutively activates the tyrosine kinase domain of the FGFR protein. In one embodiment, the FGFR protein is FGFR1, FGFR2, FGFR3, or FGFR4. In another embodiment, the fusion protein is an FGFR-TACC fusion protein. In a further embodiment, the FGFR-TACC fusion protein is FGFR1-TACC1, FGFR2-TACC2, or FGFR3-TACC3. In some embodiments, the FGFR1-TACC1 fusion protein comprises the amino acid sequence of SEQ ID NO: 150. In other embodiments, the FGFR3-TACC3 fusion protein comprises the amino acid sequence of SEQ ID NO: 79, 158, 159, 160, 161, 539, 540, 541, 542, 543, 544, 545, 546, or 547.

An aspect of the invention provides for a composition for decreasing in a subject the expression level or activity of a fusion protein comprising the tyrosine kinase domain of an FGFR protein fused to a polypeptide that constitutively activates the tyrosine kinase domain of the FGFR protein, the composition in an admixture of a pharmaceutically acceptable carrier comprising an inhibitor of the fusion protein. In one embodiment, the fusion protein is an FGFR-TACC fusion protein. In another embodiment, the inhibitor comprises an antibody that specifically binds to a FGFR-TACC fusion protein or a fragment thereof; a small molecule that specifically binds to a FGFR protein; a small molecule that specifically binds to a TACC protein; an antisense RNA or antisense DNA that decreases expression of a FGFR-TACC fusion polypeptide; a siRNA that specifically targets a FGFR-TACC fusion gene; or a combination of the listed inhibitors. In a further embodiment, the FGFR protein is FGFR1, FGFR2, FGFR3, or FGFR4. In some embodiments, the FGFR-TACC fusion protein is FGFR1-TACC1, FGFR2-TACC2, or FGFR3-TACC3. In other embodiments, the small molecule that specifically binds to a FGFR protein comprises AZD4547, NVP-BGJ398, PD173074, NF449, TK1258, BIBF-1120, BMS-582664, AZD-2171, TSU68, AB1010, AP24534, E-7080, LY2874455, or a combination of the listed small molecules. In other embodiments, the small molecule that specifically binds to a FGFR protein comprises an oral pan-FGFR tyrosine kinase inhibitor. In other embodiments, the small molecule that specifically binds to a FGFR protein comprises JNJ-42756493.

An aspect of the invention provides for a method for decreasing in a subject in need thereof the expression level or activity of a fusion protein comprising the tyrosine kinase domain of an FGFR protein fused to a polypeptide that constitutively activates the tyrosine kinase domain of the FGFR protein. In one embodiment, the method comprises administering to the subject a therapeutic amount of a composition for decreasing the expression level or activity in a subject of a fusion protein comprising the tyrosine kinase domain of an FGFR protein fused to a polypeptide that constitutively activates the tyrosine kinase domain of the FGFR protein. In one embodiment, the method comprises obtaining a sample from the subject to determine the level of expression of an FGFR fusion molecule in the subject. In some embodiments, the sample is incubated with an agent that binds to an FGFR fusion molecule, such as an antibody, a probe, a nucleic acid primer, and the like. In one embodiment, the detection or determining comprises nucleic acid sequencing, selective hybridization, selective amplification, gene expression analysis, or a combination thereof. In another embodiment, the detection or determination comprises protein expression analysis, for example by western blot analysis, ELISA, immunostaining, or other antibody detection methods. In a further embodiment, the method comprises determining whether the fusion protein expression level or activity is decreased compared to fusion protein expression level or activity prior to administration of the composition, thereby decreasing the expression level or activity of the fusion protein. In one embodiment, the fusion protein is an FGFR-TACC fusion protein. In a further embodiment, the FGFR protein is FGFR1, FGFR2, FGFR3, or FGFR4. In some embodiments, the FGFR-TACC fusion protein is FGFR1-TACC1, FGFR2-TACC2, or FGFR3-TACC3. In one embodiment, the composition for decreasing the expression level or activity of a fusion protein comprises an antibody that specifically binds to a FGFR-TACC fusion protein or a fragment thereof; a small molecule that specifically binds to a FGFR protein; a small molecule that specifically binds to a TACC protein; an antisense RNA or antisense DNA that decreases expression of a FGFR-TACC fusion polypeptide; a siRNA that specifically targets a FGFR-TACC fusion gene; or a combination of the listed inhibitors. In a further embodiment, the FGFR protein is FGFR1, FGFR2, FGFR3, or FGFR4. In some embodiments, the FGFR-TACC fusion protein is FGFR1-TACC1, FGFR2-TACC2, or FGFR3-TACC3. In other embodiments, the small molecule that specifically binds to a FGFR protein comprises AZD4547, NVP-BGJ398, PD173074, NF449, TK1258, BIBF-1120, BMS-582664, AZD-2171, TSU68, AB1010, AP24534, E-7080, LY2874455, or a combination of the small molecules listed. In other embodiments, the small molecule that specifically binds to a FGFR protein comprises an oral pan-FGFR tyrosine kinase inhibitor. In other embodiments, the small molecule that specifically binds to a FGFR protein comprises JNJ-42756493.

An aspect of the invention provides for a method for treating a gene-fusion associated cancer in a subject in need thereof, the method comprising administering to the subject an effective amount of a FGFR fusion molecule inhibitor. In one embodiment, the gene-fusion associated cancer comprises an epithelial cancer. In one embodiment, the gene-fusion associated cancer comprises glioblastoma multiforme, breast cancer, lung cancer, prostate cancer, or colorectal carcinoma. In one embodiment, the gene-fusion associated cancer comprises bladder carcinoma, squamous lung carcinoma and head and neck carcinoma. In one embodiment, the gene-fusion associated cancer comprises glioma. In one embodiment, the gene-fusion associated cancer comprises grade II or III glioma. In one embodiment, the gene-fusion associated cancer comprises IDH wild-type grade II or III glioma. In one embodiment, the method comprises obtaining a sample from the subject to determine the level of expression of an FGFR fusion molecule in the subject. In some embodiments the sample from the subject is a tissue sample. In some embodiments, the sample is a paraffin embedded tissue section. In some embodiments, the tissue sample from the subject is a tumor sample. In some embodiments, the sample is incubated with an agent that binds to an FGFR fusion molecule, such as an antibody, a probe, a nucleic acid primer, and the like. In one embodiment, the detection or determining comprises nucleic acid sequencing, selective hybridization, selective amplification, gene expression analysis, or a combination thereof. In another embodiment, the detection or determination comprises protein expression analysis, for example by western blot analysis, ELISA, immunostaining, or other antibody detection methods. In another embodiment, the FGFR fusion protein comprises an FGFR protein fused to a polypeptide that constitutively activates the tyrosine kinase domain of the FGFR protein. In one embodiment, the fusion protein is an FGFR-TACC fusion protein. In another embodiment, the inhibitor comprises an antibody that specifically binds to a FGFR-TACC fusion protein or a fragment thereof; a small molecule that specifically binds to a FGFR protein; a small molecule that specifically binds to a TACC protein; an antisense RNA or antisense DNA that decreases expression of a FGFR-TACC fusion polypeptide; a siRNA that specifically targets a FGFR-TACC fusion gene; or a combination of the listed inhibitors. In a further embodiment, the FGFR protein is FGFR1, FGFR2, FGFR3, or FGFR4. In some embodiments, the FGFR-TACC fusion protein is FGFR1-TACC1, FGFR2-TACC2, or FGFR3-TACC3. In other embodiments, the small molecule that specifically binds to a FGFR protein comprises AZD4547, NVP-BGJ398, PD173074, NF449, TK1258, BIBF-1120, BMS-582664, AZD-2171, TSU68, AB1010, AP24534, E-7080, LY2874455, or a combination of the small molecules listed. In other embodiments, the small molecule that specifically binds to a FGFR protein comprises an oral pan-FGFR tyrosine kinase inhibitor. In other embodiments, the small molecule that specifically binds to a FGFR protein comprises JNJ-42756493.

An aspect of the invention provides for a method of decreasing growth of a solid tumor in a subject in need thereof, the method comprising administering to the subject an effective amount of a FGFR fusion molecule inhibitor, wherein the inhibitor decreases the size of the solid tumor. In one embodiment, the solid tumor comprises glioblastoma multiforme, breast cancer, lung cancer, prostate cancer, or colorectal carcinoma. In one embodiment, the solid tumor comprises bladder carcinoma, squamous lung carcinoma and head and neck carcinoma. In one embodiment, the solid tumor comprises glioma. In one embodiment, the solid tumor comprises grade II or III glioma. In one embodiment, the solid tumor comprises IDH wild-type grade II or III glioma. In one embodiment, the method comprises obtaining a sample from the subject to determine the level of expression of an FGFR fusion molecule in the subject. In some embodiments the sample from the subject is a tissue sample. In some embodiments, the sample is a paraffin embedded tissue section. In some embodiments, the tissue sample from the subject is a tumor sample. In some embodiments, the sample is incubated with an agent that binds to an FGFR fusion molecule, such as an antibody, a probe, a nucleic acid primer, and the like. In one embodiment, the detection or determining comprises nucleic acid sequencing, selective hybridization, selective amplification, gene expression analysis, or a combination thereof. In another embodiment, the detection or determination comprises protein expression analysis, for example by western blot analysis, ELISA, immunostaining, or other antibody detection methods. In another embodiment, the FGFR fusion protein comprises an FGFR protein fused to a polypeptide that constitutively activates the tyrosine kinase domain of the FGFR protein. In one embodiment, the fusion protein is an FGFR-TACC fusion protein. In another embodiment, the inhibitor comprises an antibody that specifically binds to a FGFR-TACC fusion protein or a fragment thereof; a small molecule that specifically binds to a FGFR protein; a small molecule that specifically binds to a TACC protein; an antisense RNA or antisense DNA that decreases expression of a FGFR-TACC fusion polypeptide; a siRNA that specifically targets a FGFR-TACC fusion gene; or a combination of the listed inhibitors. In a further embodiment, the FGFR protein is FGFR1, FGFR2, FGFR3, or FGFR4. In some embodiments, the FGFR-TACC fusion protein is FGFR1-TACC1, FGFR2-TACC2, or FGFR3-TACC3. In other embodiments, the small molecule that specifically binds to a FGFR protein comprises AZD4547, NVP-BGJ398, PD173074, NF449, TK1258, BIBF-1120, BMS-582664, AZD-2171, TSU68, AB1010, AP24534, E-7080, LY2874455, or a combination of the small molecules listed. In other embodiments, the small molecule that specifically binds to a FGFR protein comprises an oral pan-FGFR tyrosine kinase inhibitor. In other embodiments, the small molecule that specifically binds to a FGFR protein comprises JNJ-42756493.

An aspect of the invention provides for a diagnostic kit for determining whether a sample from a subject exhibits a presence of a FGFR fusion, the kit comprising at least one oligonucleotide that specifically hybridizes to a FGFR fusion, or a portion thereof. In one embodiment, the oligonucleotides comprise a set of nucleic acid primers or in situ hybridization probes. In another embodiment, the oligonucleotide comprises SEQ ID NO: 162, 163, 164, 165, 166, 167, 168, 169, 495, 496, 497, 498, 499, 500, 501, 502, 503, 504, 505, 506, 507, 508, 509, 510 or a combination of the listed oligonucleotides. In one embodiment, the primers prime a polymerase reaction only when a FGFR fusion is present. In another embodiment, the determining comprises gene sequencing, selective hybridization, selective amplification, gene expression analysis, or a combination thereof. In a further embodiment, the FGFR-fusion is an FGFR-TACC fusion. In some embodiments, the FGFR is FGFR1, FGFR2, FGFR3, or FGFR4. In other embodiments, the FGFR-TACC fusion is FGFR1-TACC1, FGFR2-TACC2, or FGFR3-TACC3.

An aspect of the invention provides for a diagnostic kit for determining whether a sample from a subject exhibits a presence of a FGFR fusion protein, the kit comprising an antibody that specifically binds to a FGFR fusion protein comprising SEQ ID NO: 79, 85, 86, 87, 88, 89, 150, 158, 159, 160, 161, 516, 518, 539, 540, 541, 542, 543, 544, 545, 546, or 547 wherein the antibody will recognize the protein only when a FGFR fusion protein is present. In one embodiment, the antibody directed to and FGFR fusion comprising SEQ ID NO: 79, 85, 86, 87, 88, 89, 150, 158, 159, 160, 161, 516, 518 539, 540, 541, 542, 543, 544, 545, 546, or 547. In a further embodiment, the FGFR-fusion is an FGFR-TACC fusion. In some embodiments, the FGFR is FGFR1, FGFR2, FGFR3, or FGFR4. In other embodiments, the FGFR-TACC fusion is FGFR1-TACC1, FGFR2-TACC2, or FGFR3-TACC3. In some embodiments the sample from the subject is a tissue sample. In some embodiments, the sample is a paraffin embedded tissue section. In some embodiments, the tissue sample from the subject is a tumor sample.

An aspect of the invention provides for a method for detecting the presence of a FGFR fusion in a human subject. In one embodiment, the method comprises obtaining a biological sample from the human subject. In some embodiments the sample from the subject is a tissue sample. In some embodiments, the sample is a paraffin embedded tissue section. In some embodiments, the tissue sample from the subject is a tumor sample. In some embodiments, the sample is incubated with an agent that binds to an FGFR fusion molecule, such as an antibody. In another embodiment, the detection or determination comprises protein expression analysis, for example by western blot analysis, ELISA, immunostaining or other antibody detection methods. In some embodiments, the method further comprises assessing whether to administer a FGFR fusion molecule inhibitor based on the expression pattern of the subject. In further embodiments, the method comprises administering a FGFR fusion molecule inhibitor to the subject. In other embodiments, the FGFR fusion molecule inhibitor comprises an oral pan-FGFR tyrosine kinase inhibitor. In other embodiments, the FGFR fusion molecule inhibitor comprises JNJ-42756493. In another embodiment, the method comprises detecting whether or not there is a FGFR fusion present in the subject. In one embodiment, the detecting comprises measuring FGFR fusion protein levels by ELISA using an antibody directed to SEQ ID NO: 79, 85, 86, 87, 88, 89, 150, 158, 159, 160, 161, 516, 518 539, 540, 541, 542, 543, 544, 545, 546, or 547; western blot using an antibody directed to SEQ ID NO: 79, 85, 86, 87, 88, 89, 150, 158, 159, 160, 161, 516, 518 539, 540, 541, 542, 543, 544, 545, 546, or 547; immunostaining using an antibody directed to SEQ ID NO: 79, 85, 86, 87, 88, 89, 150, 158, 159, 160, 161, 516, 518, 539, 540, 541, 542, 543, 544, 545, 546, or 547; mass spectroscopy, isoelectric focusing, or a combination of the listed methods. In some embodiments, the FGFR-fusion is an FGFR-TACC fusion. In other embodiments, the FGFR is FGFR1, FGFR2, FGFR3, or FGFR4. In other embodiments, the FGFR-TACC fusion is FGFR1-TACC1, FGFR2-TACC2, or FGFR3-TACC3.

An aspect of the invention provides for a method for detecting the presence of a FGFR fusion in a human subject. In one embodiment, the method comprises obtaining a biological sample from a human subject. In some embodiments, the sample is incubated with an agent that binds to an FGFR fusion molecule, such as a probe, a nucleic acid primer, and the like. In other embodiments, the detection or determination comprises nucleic acid sequencing, selective hybridization, selective amplification, gene expression analysis, or a combination thereof. In some embodiments, the method further comprises assessing whether to administer a FGFR fusion molecule inhibitor based on the expression pattern of the subject. In further embodiments, the method comprises administering a FGFR fusion molecule inhibitor to the subject. In another embodiment, the method comprises detecting whether or not there is a nucleic acid sequence encoding a FGFR fusion protein in the subject. In one embodiment, the nucleic acid sequence comprises any one of SEQ ID NOS: 1-77, 80-84, 95-145, 515, 517, 519-527, or 530-538. In another embodiment, the detecting comprises using hybridization, amplification, or sequencing techniques to detect a FGFR fusion. In a further embodiment, the amplification uses primers comprising SEQ ID NO: 162, 163, 164,165, 166, 167, 168, 169, 495, 496, 497, 498, 499, 500, 501, 502, 503, 504, 505, 506, 507, 508, 509 or 510. In some embodiments, the FGFR-fusion is an FGFR-TACC fusion. In other embodiments, the FGFR is FGFR1, FGFR2, FGFR3, or FGFR4. In other embodiments, the FGFR-TACC fusion is FGFR1-TACC1, FGFR2-TACC2, or FGFR3-TACC3.

An aspect of the invention provides for a method of initiating oncogenic transformation in vitro. The method comprises (a) transducing cells cultured in vitro with FGFR-TACC fusion DNA; and (b) determining whether the cells acquire the ability to grow in anchorage-independent conditions, form multi-layered foci, or a combination thereof.

An aspect of the invention provides for a method of initiating oncogenic transformation in vivo. The method comprises (a) transducing cells cultured in vitro with FGFR-TACC fusion DNA; (b) injecting a mouse with the transduced cells; and (c) determining whether a tumor grows in the mouse. In one embodiment, the injecting is a subcutaneous or intracranial injection.

An aspect of the invention provides a method of identifying a compound that decreases the oncogenic activity of a FGFR-TACC fusion. The method comprises (a) transducing a cell cultured in vitro with FGFR-TACC DNA; (b) contacting a cell with a ligand source for an effective period of time; and (c) determining whether the cells acquire the ability to grow in anchorage-independent conditions, form multi-layered foci, or a combination thereof, compared to cells cultured in the absence of the test compound.

In one embodiment, the method can comprise contacting a sample from the subject with an antibody specific for a FGFR fusion molecule, and determining the presence of an immune complex. In another embodiment, the method can comprise contacting a sample from the subject with an antibody specific for a FGFR molecule, or a TACC molecule, and determining the presence of an immune complex. In another embodiment, the antibody can recognize the FGFR3 C-terminal region, or the TACC3 N-terminal region, or a combination thereof. In another embodiment, the antibody can recognize the FGFR3 C-terminal region, or the TACC3 N-terminal region, or a combination thereof. In another embodiment, the method can comprise contacting a sample from the subject with an antibody specific for a FGFR molecule, or a TACC molecule, or a FGFR fusion molecule, and determining the amount of an immune complex formed compared to the amount of immune complex formed in non-tumor cells or tissue, wherein an increased amount of an immune complex indicates the presence of an FGFR fusion.

In one embodiment, the method can comprise contacting a sample from the subject with primers specific for a FGFR fusion molecule, and determining the presence of an PCR product. In another embodiment, the method can comprise contacting a sample from the subject with primer specific for a FGFR molecule, or a TACC molecule, and determining the presence of a PCR product. In another embodiment, the primers can recognize the nucleic acids encoding a FGFR3 C-terminal region, or nucleic acids encoding a TACC3 N-terminal region, or a combination thereof. In another embodiment, the method can comprise contacting a sample from the subject with primers specific for a FGFR molecule, or a TACC molecule, or a FGFR fusion molecule, and determining the amount of PCR product formed compared to the amount of PCR product formed in non-tumor cells or tissue, wherein an increased amount of PCR product indicates the presence of an FGFR fusion.

An aspect of the invention provides for a purified fusion protein comprising the tyrosine kinase domain of an FGFR protein fused to the TACC domain of a transforming acidic coiled-coil-containing (TACC) protein. In one embodiment, the FGFR protein is FGFR1, FGFR2, FGFR3, or FGFR4. In one embodiment, the TACC protein is TACC1, TACC2, or TACC3. In one embodiment, the fusion protein is FGFR1-TACC1, FGFR2-TACC2, or FGFR3-TACC3. In one embodiment, the fusion protein comprises SEQ ID NO: 79, SEQ ID NO: 158, SEQ ID NO: 159, SEQ ID NO: 160, SEQ ID NO: 161, SEQ ID NO: 539, SEQ ID NO: 540, SEQ ID NO: 541, SEQ ID NO: 542, SEQ ID NO: 543, SEQ ID NO: 545, SEQ ID NO: 546, or SEQ ID NO: 547. In one embodiment, the fusion protein has a breakpoint comprising at least 3 consecutive amino acids from amino acids 730-758 of SEQ ID NO: 90 and comprising at least 3 consecutive amino acids from amino acids 549-838 of SEQ ID NO: 92. In one embodiment, the fusion protein has a breakpoint comprising SEQ ID NO: 78, SEQ ID NO: 85, SEQ ID NO: 86, SEQ ID NO: 87, SEQ ID NO:89, SEQ ID NO: 516, or SEQ ID NO:518. In one embodiment, the fusion protein comprises SEQ ID NO: 150. In one embodiment, the fusion protein has a breakpoint comprising at least 3 consecutive amino acids from amino acids 746-762 of SEQ ID NO: 146 and comprising at least 3 consecutive amino acids from amino acids 572-590 of SEQ ID NO: 148. In one embodiment, thw fusion protein has a breakpoint comprising SEQ ID NO: 88.

An aspect of the invention provides for a cDNA encoding a fusion protein comprising the tyrosine kinase domain of FGFR fused to the TACC domain of TACC. In one embodiment the FGFR is FGFR1, FGFR2, FGFR3, or FGFR4. In one embodiment, the TACC is TACC1, TACC2, or TACC3. In one embodiment, the fusion protein is FGFR1-TACC1, FGFR2-TACC2, or FGFR3-TACC3. In one embodiment, the cDNA comprises SEQ ID NO: 94, SEQ ID NO: 530, SEQ ID NO: 531, SEQ ID NO: 532, SEQ ID NO: 533, SEQ ID NO: 534, SEQ ID NO: 535, SEQ ID NO: 536, SEQ ID NO: 537 or SEQ ID NO: 538. In one embodiment, the cDNA has a breakpoint comprising at least 9 consecutive in-frame nucleotides from nucleotides 2443-2530 of SEQ ID NO: 91 and comprising at least 9 consecutive in-frame nucleotides from nucleotides 1800-2847 of SEQ ID NO: 93. In one embodiment, the cDNA has a breakpoint comprising any one of SEQ ID NOs: 1-77, or SEQ ID NOs: 519-527. In one embodiment, the cDNA comprises SEQ ID NO: 151. In one embodiment, the cDNA has a breakpoint comprising at least 9 consecutive in-frame nucleotides from nucleotides 3178-3228 of SEQ ID NO: 147 and comprising at least 9 consecutive in-frame nucleotides from nucleotides 2092-2794 of SEQ ID NO: 149. In one embodiment, the cDNA has a breakpoint comprising SEQ ID NO: 83. In one embodiment, the cDNA comprises a combination of exons 1-16 of FGFR3 spliced 5′ to a combination of exons 8-16 of TACC3, wherein a breakpoint occurs in: a) any one of exons 1-16 of FGFR3 and any one of exons 8-16 of TACC3; b) any one of introns 1-16 of FGFR3 and any one of exons 8-16 of TACC3; c) any one of exons 1-16 of FGFR3 and any one of introns 7-16 of TACC3; or d) any one of introns 1-16 of FGFR3 and any one of introns 7-16 of TACC3. In one embodiment, the cDNA comprises a combination of exons 1-17 of FGFR1 spliced 5′ to a combination of exons 7-13 of TACC1, wherein a breakpoint occurs in any one of exons 1-17 of FGFR3 and any one of exons 7-13 of TACC3. In one embodiment, the cDNA comprises a combination of exons 1-18 of FGFR2 spliced 5′ to a combination of exons 1-23 of TACC2.

### BRIEF DESCRIPTION OF THE FIGURES

This patent or application file contains at least one drawing executed in color. Copies of this patent or patent application publication with color drawing(s) will be provided by the Office upon request and payment of the necessary fee.

FIG. 1A is a graph that shows genes recurrently involved in gene fusions in TCGA. Only genes involved in at least three gene fusions across different samples are displayed.

FIGS. 1B-1, 1B-2, 1B-3 and 1B-4 show an FGFR3-TACC3 gene fusion identified by whole transcriptome sequencing of GSCs. 76 split-reads (SEQ ID NOS: 2-77, respectively) are shown aligning on the breakpoint. The predicted reading frame at the breakpoint is shown at the top (FGFR3 nucleotide sequence (left) and TACC3 nucleotide sequence (right); SEQ ID NO: 1) with FGFR3 sequences below the predicted reading frame (left) and TACC3 (right). The putative amino acid sequence (SEQ ID NO: 78) corresponding to SEQ ID NO: 1 is shown above the predicted reading frame.

FIG. 1C shows an FGFR3-TACC3 gene fusion identified by whole transcriptome sequencing of GSCs. On the left, FGFR3-TA CC3-specific PCR from cDNA derived from GSCs and GBM is shown. On the right, Sanger sequencing chromatogram shows the reading frame at the breakpoint (SEQ ID NO: 80) and putative translation of the fusion protein (SEQ ID NO: 85) in the positive samples.

FIG. 1D shows an FGFR3-TACC3 gene fusion identified by whole transcriptome sequencing of GSCs. Amino acid sequence of the FGFR3-TACC3 protein is shown (SEQ ID NO: 79). Residues corresponding to FGFR3 or to TACC3 (underlined) are shown. The fusion protein joins the tyrosine kinase domain of FGFR3 to the TACC domain of TACC3.

FIG. 1E shows an FGFR3-TACC3 gene fusion identified by whole transcriptome sequencing of GSCs. Genomic fusion of FGFR3 exon 17 with intron 7 of TACC3 is shown. In the fused mRNA, exon 16 of FGFR3 is spliced 5′ to exon 8 of TACC3. Filled arrows indicate the position of the fusion-genome primers, which generate fusion-specific PCR products in GSC-1123 and GBM-1123.

FIG. 2A shows recurrent gene fusions between FGFR and TACC genes in GBM. Specifically, FGFR3-TACC3 gene fusions are shown that were identified by exome sequencing analysis. Split-reads are shown aligning the genomic breakpoints of FGFR3 and TACC3 genes in the four TCGA GBM samples. For TCGA-27-1835, SEQ ID NO: 95 shows the reading frame at the breakpoint (bold), while SEQ ID NOS: 96-107, respectively, show alignments of the genomic breakpoints. For TCGA-19-5958, SEQ ID NO: 108 shows the reading frame at the breakpoint (bold), while SEQ ID NOS: 109-111, respectively, show alignments of the genomic breakpoints. For TCGA-06-6390, SEQ ID NO: 112 shows the reading frame at the breakpoint (bold), while SEQ ID NOS: 113-131, respectively, show alignments of the genomic breakpoints. For TCGA-12-0826, SEQ ID NO: 132 shows the reading frame at the breakpoint (bold), while SEQ ID NOS: 133-145, respectively, show alignments of the genomic breakpoints.

FIG. 2B shows recurrent gene fusions between FGFR and TACC genes in GBM. On the left, a gel of FGFR-TACC-specific PCR is shown for FGFR3-TACC3 from a GBM cDNA sample. On the right, Sanger sequencing chromatograms show the reading frame at the breakpoint (SEQ ID NO: 81) and putative translation of the fusion protein (SEQ ID NO: 86) in the positive samples.

FIG. 2C shows recurrent gene fusions between FGFR and TACC genes in GBM. On the left, a gel of FGFR-TACC-specific PCR is shown for FGFR3-TACC3 from a GBM cDNA sample. On the right, Sanger sequencing chromatograms show the reading frame at the breakpoint (SEQ ID NO: 82) and putative translation of the fusion protein (SEQ ID NO: 87) in the positive samples.

FIG. 2D shows recurrent gene fusions between FGFR and TACC genes in GBM. Co-outlier expression of FGFR3 and TACC3 in four GBM tumors from Atlas-TCGA is shown in the plot.

FIG. 2E shows recurrent gene fusions between FGFR and TACC genes in GBM. CNV analysis shows micro-amplifications of the rearranged portions of the FGFR3 and TACC3 genes in the same four Atlas-TCGA GBM samples.

FIG. 2F shows recurrent gene fusions between FGFR and TACC genes in GBM. On the left, a gel of FGFR-TACC-specific PCR is shown for FGFR1-TACC1 from a GBM cDNA sample. On the right, Sanger sequencing chromatograms show the reading frame at the breakpoint (SEQ ID NO: 83) and putative translation of the fusion protein (SEQ ID NO: 88) in the positive samples.

FIG. 2G shows recurrent gene fusions between FGFR and TACC genes in GBM. On the left, a gel of FGFR-TACC-specific PCR is shown for FGFR3-TACC3 from a GBM cDNA sample. On the right, Sanger sequencing chromatograms show the reading frame at the breakpoint (SEQ ID NO: 84) and putative translation of the fusion protein (SEQ ID NO: 89) in the positive samples.

FIG. 3A shows transforming activity of FGFR-TACC fusion proteins. FGFR1-TACC1 and FGFR3-TACC3 induce anchorage-independent growth in Rat1A fibroblasts. The number of soft agar colonies was scored from triplicate samples 14 days after plating. Representative microphotographs are shown.

FIG. 3B are photomicrographs showing of immunofluoresence staining of tumors from mice injected with Ink4A;Arf−/− astrocytes expressing FGFR3-TACC3 showing positivity for glioma-specific (Nestin, Oig2 and GFAP) and proliferation markers (Ki67 and pHH3). Sub-cutaneous tumors were generated by Ink4A;Arf−/− astrocytes expressing FGFR-TACC fusions.

FIG. 3C shows Kaplan-Meier survival curves of mice injected intracranially with pTomo-shp53 (n=8) or pTomo-EGFRvII-shp53 (n=7) (green line; “light grey” in black and white image) and pTomo-FGFR3-TACC3-shp53 (n=8, red line; “dark grey” in black and white image). Points on the curves indicate deaths (log-rank test, p=0.00001, pTomo-shp53 vs. pTomo-FGFR3-TACC3-shp53).

FIG. 3D shows representative photomicrographs of Hematoxylin and Eosin staining of advanced FGFR3-TACC3-shp53 generated tumors showing histological features of high-grade glioma. Of note is the high degree of infiltration of the normal brain by the tumor cells. Immunofluorescence staining shows that glioma and stem cell markers (Nestin, Olig2 and GFAP), the proliferation markers (Ki67 and pHH3) and the FGFR3-TACC3 protein are widely expressed in the FGFR3-TACC3-shp53 brain tumors. F1-T1: FGFR1-TACC1; F3-T3: FGFR3-TACC3; F3-T3-K508M: FGFR3-TACC3-K508M.

FIG. 4A shows that FGFR3-TACC3 localizes to spindle poles, delays mitotic progression and induces chromosome segregation defects and aneuploidy Constitutive auto-phosphorylation of FGFR3-TACC3 fusion. Ink4A;Arf−/− astrocytes transduced with empty lentivirus or a lentivirus expressing FGFR3-TACC3 or FGFR3-TACC3-K508M were left untreated (0) or treated with 100 nM of the FGFR inhibitor PD173074 for the indicated times. Phospho-proteins and total proteins were analyzed by Western blot using the indicated antibodies.

FIG. 4B shows that FGFR3-TACC3 localizes to spindle poles, delays mitotic progression and induces chromosome segregation defects. Photomicrographs are shown of confocal microscopy analysis of FGFR3-TACC3 in Ink4A;Arf−/− astrocytes. Maximum intensity projection of z-stacked images shows FGFR3-TACC3 (red; “dark grey” in black and white image) coating the spindle poles of a representative mitotic cell (upper panels). In telophase (lower panels) FGFR3-TACC3 localizes to the mid-body. α-tubulin (green; “grey” in black and white image), DNA (DAPI, blue; “light grey” in black and white image).

FIG. 4C shows representative fluorescence video-microscopy for cells transduced with vector or FGFR3-TACC3.

FIG. 4D shows a Box and Whisker plot representing the analysis of the time from nuclear envelope breakdown (NEB) to anaphase onset and from NEB to nuclear envelope reconstitution (NER). The duration of mitosis was measured by following 50 mitoses for each condition by time-lapse microscopy.

FIG. 4E shows that FGFR3-TACC3 localizes to spindle poles, delays mitotic progression and induces chromosome segregation defects. Representative images are shown of cells with chromosome missegregation. Arrows point to chromosome misalignments, lagging chromosomes, and chromosome bridges.

FIG. 4F shows quantitative analysis of segregation defects in Rat1A expressing FGFR1-TACC1 and FGFR3-TACC3. F3-T3: FGFR3-TACC3; F3-T3-K508M: FGFR3-TACC3-K508M.

FIG. 5A shows karyotype analysis of Rat1A cells transduced with control, FGFR3, TACC3 or FGFR3-TACC3 expressing lentivirus. Distribution of chromosome counts of cells arrested in mitosis and analyzed for karyotypes using DAPI. Chromosomes were counted in 100 metaphase cells for each condition to determine the ploidy and the diversity of chromosome counts within the cell population. FGFR3-TACC3 fusion induces aneuploidy.

FIG. 5B shows representative karyotypes and FIG. 5C shows distribution of chromosome counts of human astrocytes transduced with control or FGFR3-TACC3 expressing lentivirus. Chromosomes were counted in 100 metaphase cells for each condition to determine the ploidy and the diversity of chromosome counts within the cell population.

FIG. 5D shows quantitative analysis of chromosome number in 100 metaphase cells for each condition to determine the ploidy and the diversity of chromosome counts within the cell population. (n=3 independent experiments).

FIG. 6A shows inhibition of FGFR-TK activity corrects the aneuploidy initiated by FGFR3-TACC3. The upper panel is a karyotype analysis of Rat1A cells transduced with control or FGFR3-TACC3 lentivirus and treated with vehicle (DMSO) or PD173470 (100 nM) for five days. The lower panel shows the ploidy and the diversity of chromosome counts within the cell population were determined by quantitative analysis of chromosome number in 100 metaphase cells for each condition.

FIG. 6B shows inhibition of FGFR-TK activity corrects the aneuploidy initiated by FGFR3-TACC3. Correction of premature sister chromatid separation (PMSCS) by PD173470 in cells expressing FGFR3-TACC3. Panels show representative metaphase spreads. DNA was stained by DAPI. FIG. 6C shows quantitative analysis of metaphases with loss of sister chromatid cohesion (n=3; p=0.001, FGFR3-TACC3 treated with DMSO vs. FGFR3-TACC3 treated with PD173470).

FIG. 7A shows inhibition of FGFR-TK activity suppresses tumor growth initiated by FGFR3-TACC3. Growth rate of Rat1A transduced with the indicated lentiviruses and treated for three days with increasing concentrations of PD173074. Cell growth was determined by the MTT assay. Data are presented as the means±standard error (n=4).

FIG. 7B shows the growth rate of GSC-1123 treated with PD173470 at the indicated concentrations for the indicated times. Cell growth was determined by the MTT assay. Data are presented as the means±standard error (n=4).

FIG. 7C shows the growth inhibitory effect of silencing FGFR3-TACC3 fusion. At the left, parallel cultures of GSC-1123 cells were transduced in triplicate. Rat1A cells expressing FGFR3-TACC3 fusion were transduced with lentivirus expressing a non-targeting shRNA (Ctr) or shRNA sequences targeting FGFR3 (sh2, sh3, sh4). Five days after infection cells were plated at density of 2×10⁴ cells/well in triplicate and the number of trypan blue excluding cells was scored at the indicated times. Infection with lentivirus expressing sh-3 and sh-4, the most efficient FGFR3 silencing sequences reverted the growth rate of FGFR3-TACC3 expressing cultures to levels comparable to those of Rat1A transduced with empty vector. Values are the means±standard deviation (n=3). At th right sided figure, GSC-1123 cells were transduced with lentivirus expressing a non-targeting shRNA (sh-Ctr) or lentivirus expressing sh-3 and sh-4 sequences targeting FGFR3. Western Blot analysis was performed on parallel cultures using the FGFR3 antibody to the detect FGFT3-TACC3 fusion protein. β-actin is shown as a control for loading.

FIG. 7D shows that the FGFR inhibitor PD173074 suppresses tumor growth of glioma sub-cutaneous xenografts generated by Ink4A;Arf−/− astrocytes expressing FGFR3-TACC3. After tumor establishment (200-300 mm³, arrow) mice were treated with vehicle or PD173074 (50 mg/kg) for 14 days. Values are mean tumor volumes ±standard error (n=7 mice per group).

FIG. 7E is a Kaplan-Meier analysis of glioma-bearing mice following orthotopic implantation of Ink4A;Arf−/− astrocytes transduced with FGFR3-TACC3. After tumor engraftment mice were treated with vehicle (n=9) or AZD4547 (50 mg/kg) (n=7) for 20 days (p=0.001).

FIG. 8 shows a schematic of the TX-Fuse pipeline for the identification of fusion transcripts from RNA-Seq data generated from nine GSC cultures. The continued figure shows a schematic of the Exome-Fuse pipeline for the identification of gene fusion rearrangements from DNA exome sequences of 84 GBM TCGA tumor samples.

FIGS. 9A-D shows the validation of fusion transcripts identified by RNA-seq of nine GSCs. Sanger sequencing chromatograms show the reading frames at the breakpoint and putative translation of the fusion proteins in the positive samples (right side). The left side shows gels of RT-PCR conducted. (A) POLR2A-WRAP53. DNA sequence disclosed as SEQ ID NO: 319 and protein sequence disclosed as SEQ ID NO: 320. (B) CAPZB-UBR4. DNA sequence disclosed as SEQ ID NO: 321 and protein sequence disclosed as SEQ ID NO: 322. (C) ST8SIA4-PAM. DNA sequence disclosed as SEQ ID NO: 323 and protein sequence disclosed as SEQ ID NO: 324. (D) PIGU-NCOA6. DNA sequence disclosed as SEQ ID NO: 325 and protein sequence disclosed as SEQ ID NO: 326.

FIGS. 9E-1, 9E-2, 9E-3, 9E-4, 9E-5, 9E-6, 9E-7, and 9E-8 show the fusion transcripts identified by whole transcriptome sequencing of nine GSCs. 54 split-reads (SEQ ID NOS 329-382, respectively, in order of appearance) are shown aligning on the breakpoint of the POLR2A-WRAP53 fusion (SEQ ID NO: 327). The predicted reading frame at the breakpoint is shown at the top with POLR2A sequences in red (left) and WRAP53 in blue (right). Protein sequence disclosed as SEQ ID NO: 328. On the continued page, 48 split-reads (SEQ ID NOS 385-432, respectively, in order of appearance) are shown aligning on the breakpoint of the CAPZB-UBR4 fusion (SEQ ID NO: 383). The predicted reading frame at the breakpoint is shown at the top with CAPZB sequences in red (left) and UBR4 in blue (right). Protein sequence disclosed as SEQ ID NO: 384. On the continued page after, 29 split-reads (SEQ ID NOS 435-463, respectively, in order of appearance) are shown aligning on the breakpoint of the ST8SIA4-PAM fusion (SEQ ID NO: 433). The predicted reading frame at the breakpoint is shown at the top with ST8SIA4 sequences in red (left) and PAM in blue (right). Protein sequence disclosed as SEQ ID NO: 434. On the subsequent continued page, 17 split-reads (SEQ ID NOS 466-482, respectively, in order of appearance) are shown (top) aligning on the breakpoint of the PIGU-NCOA6 fusion (SEQ ID NO: 464). The predicted reading frame at the breakpoint is shown at the top with PIGU sequences in red (left) and NCOA6 in blue (right). Protein sequence disclosed as SEQ ID NO: 465. Also (below), 6 split-reads (SEQ ID NOS 485-490, respectively, in order of appearance) are shown aligning on the breakpoint of the IFNAR2-IL10RB fusion (SEQ ID NO: 483). The predicted reading frame at the breakpoint is shown at the top with IFNAR2 sequences in red (left) and IL10RB in blue (right). Protein sequence disclosed as SEQ ID NO: 484.

FIG. 10A shows the analysis and validation of the expression of fused transcripts in GSCs and GBM sample. Expression measured by read depth from RNA-seq data. Light grey arcs indicate predicted components of transcripts fused together. Overall read depth (blue; “grey” in black and white image) and split insert depth (red; “dark grey” in black and white image) are depicted in the graph, with a 50-read increment and a maximum range of 1800 reads. Note the very high level of expression in the regions of the genes implicated in the fusion events, particularly for FGFR3-TACC3.

FIG. 10B shows the analysis and validation of the expression of fused transcripts in GSCs and GBM sample. Top panel, qRT-PCR showing the very high expression of FGFR3 and TACC3 mRNA sequences included in the FGFR3-TACC3 fusion transcript in GSC-1123. Bottom panel, for comparison the expression of sequences of WRAP53 mRNA included in the POLR2A-WRAP53 fusion in GSC-0114 is also shown.

FIG. 10C shows the expression of the FGFR3-TACC3 protein in GSC-1123 and GBM-1123. Western blot analysis with a monoclonal antibody, which recognizes the N-terminal region of human FGFR3 shows expression of a ˜150 kD protein in GSC-1123 but not in the GSC cultures GSC-0331 and GSC-0114, which lack the FGFR3-TACC3 rearrangement.

FIG. 10D shows the analysis and validation of the expression of fused transcripts in GSCs and GBM sample. Immunostaining analysis with the FGFR3 antibody of the tumor GBM-1123 (top panel) and a GBM tumor lacking the FGFR3-TACC3 rearrangement. FGFR3 (red; “light grey” in black and white image), DNA (DAPI, blue; “grey” in black and white image). The pictures were taken at low (left) and high (right) magnification.

FIGS. 10E-1, 10E-2, 10E-3, 10E-4, 10E-5, and 10E-6 shows MS/MS analysis of the ˜150 kD fusion protein immunoprecipitated by the monoclonal anti-FGFR3 antibody from GSC-1123, identifying three unique peptides mapping to the FGFR3 (FGFR3 Peptide 1 (SEQ ID NO: 492), 2 (SEQ ID NO: 493), and 3 (SEQ ID NO: 494)) and three peptides mapping to the C-terminal region of TACC3 (TACC Peptide 1 (SEQ ID NO: 156), 2 (SEQ ID NO: 157), and 3 (SEQ ID NO: 491)).

FIGS. 11A-C shows Rat1A cells transduced with control lentivirus or lentivurus expressing FGFR3, TACC3, FGFR3-TACC3 (FIG. 11A) that were analyzed by Western blot with an antibody recognizing the N-terminus of FGFR3 (included in the FGFR3-TACC3 fusion protein) or the N-terminus of TACC3 (not included in the FGFR3-TACC3 fusion protein). FIG. 11B shows quantitative Western blot analysis of endogenous FGFR3-TACC3 in GSC-1123 compared with lentivirally expressed FGFR3-TACC3 in Rat1A. FIG. 11C shows Western blot analysis of FGFR3-TACC3 and FGFR3-TACC3-K508M in Rat1A. α-tubulin is shown as a control for loading.

FIGS. 11D-F shows expression analyses of FGFR3-TACC3 fusion construct (FIG. 11D) FGFR3 immunostaining of GBM-1123 (left, upper panel), BTSC1123 (right, upper panel), mouse GBM induced by FGFR3-TACC3 expressing lentivirus (left, lower panel), and sub-cutaneous xenograft of mouse astrocytes transformed by FGFR3-TACC3 fusion (right, lower panel); FGFR3-TACC3, red (“light grey” in black and white image); DNA (DAPI), blue (“grey” in black and white image). FIG. 11E shows quantification of FGFR3-TACC3 positive cells in the tumors and cultures of cells shown in FIG. 11D. FIG. 11F shows a quantitative Western blot analysis of ectopic FGFR3-TACC3 fusion protein in mouse astrocytes and FGFR3-TACC3 induced mouse GBM (mGBM-15 and mGBM-17) compared with the endogenous expression in GBM1123. β-actin is shown as a control for loading. F3-T3: FGFR3-TACC3. α-tubulin or β-actin is shown as a control for loading.

FIG. 12A shows a western blot. Ink4A;Arf−/− astrocytes transduced with empty lentivirus or a lentivirus expressing FGFR3-TACC3 were starved of mitogens and left untreated (time 0) or treated with FGF-2 at concentration of 50 ng/ml for the indicated times. Phospho-proteins and total proteins were analyzed by Western blot using the indicated antibodies. α-tubulin is shown as a control for loading.

FIG. 12B show western blots. Ink4A;Arf−/− astrocytes transduced with empty lentivirus or a lentivirus expressing FGFR3-TACC3 or FGFR3-TACC3-K508M were starved of mitogens and left untreated (time 0) or treated for 10 min with FGF-1 at the indicated concentrations. Phospho-proteins and total proteins were analyzed by Western blot using the indicated antibodies. β-actin is shown as a control for loading.

FIG. 12C show western blots. Ink4A;Arf−/− astrocytes transduced with empty lentivirus or a lentivirus expressing FGFR3-TACC3 or FGFR3-TACC3-K508M were starved of mitogens and left untreated (time 0) or treated for 10 min with FGF-8 at the indicated concentrations. Phospho-proteins and total proteins were analyzed by Western blot using the indicated antibodies. β-actin is shown as a control for loading.

FIGS. 12D-F shows mitotic localization of FGFR3-TACC3 fusion protein. FIG. 12D shows maximum intensity projection confocal image of a representative FGFR3-TACC3 expressing Ink4A;Arf−/− mouse astrocyte at metaphase immunostained using the FGFR3 antibody (red; “dark grey” in black and white image). FGFR3-TACC3 displays asymmetric localization on top of one spindle pole. FIG. 12E shows maximum intensity projection confocal image of a representative TACC3 expressing Ink4A;Arf−/− mouse astrocyte at metaphase immunostained with the TACC3 antibody (red; (“dark grey” in black and white image). TACC3 staining coincides with the spindle microtubules. FIG. 12F shows maximum intensity projection confocal image of a representative FGFR3 expressing Ink4A;Arf−/− mouse astrocyte at metaphase immunostained with the FGFR3 antibody (red; (“dark grey” in black and white image). FGFR3 does not show a specific staining pattern in mitosis. Cells were co-immunostained using α-tubulin (green; (“light grey” in black and white image) to visualize the mitotic spindle. DNA was counterstained with DAPI (blue; (“grey” in black and white image). Images were acquired at 0.250 μm intervals. Endogenous levels of FGFR3 or TACC3 were undetectable under the applied experimental conditions. F3-T3: FGFR3-TACC3.

FIG. 13A shows sthat the FGFR3-TACC3 protein induces chromosomal mis-segregation, chromatid cohesion defects and defective spindle checkpoint. Quantitative analysis of metaphase spreads for chromosome segregation defects in Ink4A;ARF−/− astrocytes expressing vector control or FGFR3-TACC3 (upper panel). Microscope imaging analysis of chromosome segregation defects in Ink4A;Arf−/− astrocytes expressing FGFR3-TACC3 or vector control. Representative images of cells with chromosome missegregation. Arrows point to chromosome misalignments, lagging chromosomes and chromosome bridges.

FIGS. 13B-D shows representative images of premature sister chromatid separation (PMSCS) in Ink4A;Arf−/− astrocytes (FIG. 13B) and Rat1A cells (FIG. 13C) expressing FGFR3-TACC3. Left, panels show representative metaphase spreads. Right, quantitative analysis of metaphases with loss of sister chromatid cohesion. The number of mitosis with PMSCS in Ink4A;Arf−/− astrocytes was scored in at least 100 methaphases for each condition in three independent experiments. The number of mitosis with PMSCS was scored in triplicate samples of Rat1A cells. FIG. 13D is a graph showing nocodazole was added for the indicated durations to Rat1A-H2B-GFP cells transduced with the specified lentiviruses. The mitotic index at each time point was determined by quantitating the H2B-GFP-positive cells in mitosis at each time point. Data are presented as average and standard deviation (n=3). F3-T3: FGFR3-TACC3.

FIGS. 14A-B shows growth curves of human primary astrocytes transduced with lentivirus expressing FGFR3-TACC3 fusion or the empty vector. An analysis was conducted of FGFR3-TACC3 fusion mediated growth alteration and specific effect of RTK inhibitors on cells carrying FGFR-TACC fusions. FIG. 14A is a graph that shows cell proliferation of human primary astrocytes transduced with lentivirus expressing FGFR3-TACC3 fusion or the empty vector was determined by the MTT assay 7 days after infection (passage 1). Values are the means±standard deviation (n=4). p-value: 0.0033. FIG. 14B is a graph that shows cell proliferation of human primary astrocytes transduced with lentivirus expressing FGFR3-TACC3 fusion or the empty vector was determined by the MTT assay six weeks after the infection (passage 10). Values are the means±standard deviation (n=4). p-value: 0.0018.

FIGS. 14C-D shows specific growth inhibitory effect by FGFR inhibitors on FGFR-TACC fusion expressing cells. Cell growth was determined by MTT assay. Rat1A cells transduced with the indicated lentivirus were treated for three days with BGJ398 (FIG. 14C) or AZD4547 (FIG. 14D) at the indicated concentration. Values are the means±standard error (n=4).

FIG. 14E shows the growth inhibitory effect of silencing FGFR3-TACC3 fusion. (left) GSC-1123 cells were transduced in triplicate with lentivirus expressing a non-targeting shRNA (Ctr) or lentivirus expressing sh-3 and sh-4 sequences targeting FGFR3. Five days after infection cells were plated at density of 2×10⁴ cells/well in triplicate and the number of trypan blue excluding cells was scored at the indicated times. Values are the means±standard deviation (n=3). (right) Western Blot analysis was performed on parallel cultures collected five days after infection using the FGFR3 antibody to the detect FGFT3-TACC3 fusion protein. β-actin is shown as a control for loading. (**: p-value=<0.005; ***: p-value=<0.0001).

FIG. 15 shows a survival plot of cells treated with PD173074, NVP-BGJ398, or AZD4547.

FIG. 16 shows an FGFR3-TACC3 gene fusion identified by whole transcriptome sequencing of GSCs. The histogram describes the absolute frequency of each forward and reverse sequence read spanning the breakpoint.

FIG. 17 shows transforming activity of FGFR3-TACC3. FGFR3-TACC3 induces anchorage-independent growth in Rat1A fibroblasts (top panels) and a transformed phenotype in Ink4A;Arf−/− primary astrocytes (bottom panels).

FIG. 18 shows transforming activity of FGFR3-TACC3. Kaplan-Meier survival curves of mice injected intracranially with pTomo-shp53 (n=8), pTomo-FGFR3-TACC3-shp53 (n=8) and pTomo-EGFRvII-shp53 (n=7) are shown. Points on the curves indicate deaths (log-rank test, p=0.025, pTomo-shp53 vs. pTomo-FGFR3-TACC3-shp53).

FIG. 19 shows that inhibition of FGFR-TK activity corrects the aneuploidy and suppresses tumor growth initiated by FGFR3-TACC3. Short-term growth inhibition assays are shown of Rat1A transduced with the indicated lentivirus and treated with PD173470 at the indicated concentrations. Cells were treated for three days. Cell viability was determined by the MTT assay. Error bars show means±standard error (n=4).

FIG. 20 is a growth inhibition assay of human astrocytes transduced with the indicated lentivirus and treated for four days with PD173470 at the indicated concentration. Cell viability was determined by the MTT assay. Error bars show means±standard error (n=4).

FIG. 21 is a graph showing a growth inhibition assay of human astrocytes transduced with the indicated lentivirus and treated for four days with PD173470 at the indicated concentration. Cell viability was determined by the MTT assay. Error bars show means±standard error (n=4).

FIG. 22 shows graphs of the survival of Rat1A cells in short-term growth inhibition assays. (Top graph) Rat1A cells were transduced with the indicated ptomo constructs and treated with PD173074 at the indicated concentrations. Cells were treated for three days. Cell viability was determined by the MTT assay. Error bars show means±standard error (n=4). In the bottom panel, a western blot photograph is shown.

FIG. 23 shows that inhibition of FGFR-TK activity corrects the aneuploidy and suppresses tumor growth initiated by FGFR3-TACC3. A plot is shown of karyotype analysis of Rat1A cells transduced with control or FGFR3-TACC3 lentivirus and treated with vehicle (DMSO) or PD173470 (100 nM) for five days.

FIG. 24 shows Survival of glioma-bearing mice was tracked following intracranial implantation of Ink4A;Arf−/− astrocytes transduced with FGFR3-TACC3. After tumor engraftment mice were treated with vehicle or AZD4547 (50 mg/kg) for 20 days (vehicle, n=7; AZD4547, n=6; p=0.001).

FIG. 25 shows the position of the peptides from FIGS. 10E1-10E6 in the amino acid sequence of the FGFR3-TACC3 fusion protein (SEQ ID NO: 79), which are highlighted in pink (FGFR3; underlined) and blue (TACC3; dotted lines).

FIG. 26 shows Kaplan-Meier analysis of IDH mutant and FGFR3-TACC3 positive human GBM. Log rank test p-value: 0.0169.

FIGS. 27A-B are pictures that shows tumor xenografts that were induced following sub-cutaneous injection of Ink4A;Arf−/− mouse astrocytes transduced with lentivirus expressing FGFR3-TACC3 (upper panel A, right flank) or FGFR1-TACC1 (lower panel B, right flank) fusion, but not with the empty vector (upper panel, left flank) or FGFR3-TACC3 carrying a K508M mutation in the kinase domain (FGFR3-TACC3-K508M; lower panel, left flank).

FIG. 28 shows constitutive auto-phosphorylation of FGFR3-TACC3 fusion. BTSC derived from FGFR3-TACC3 or RasV12 induced mouse GBM were left untreated or treated with 500 nM PD173074 for the indicated times. Phospho-proteins and total proteins were analyzed by Western blot using the indicated antibodies. β-actin is shown as a control for loading.

FIG. 29 shows Z-stacked confocal images of the representative FGFR3-TACC3 expressing Ink4A;Arf−/− mouse astrocyte shown as a maximum intensity projection. Cells were immunostained using FGFR3 (red; “dark grey” in black and white image) and α-tubulin (green; (“light grey” in black and white image). DNA was counterstained with DAPI (blue; (“grey” in black and white image). Images were acquired at 0.250 μm intervals. Coordinates of the image series are indicated. F3-T3: FGFR3-TACC3.

FIG. 30 shows examples of SKY karyotype analysis painting two different cells from the same culture of GSC-1123, illustrating the ongoing CIN and aneuploidy. Details of the karyotype analysis of 20 cells are reported in Table 6.

FIGS. 31-1, 31-2, and 31-3 are a graphical representation of segmented CNVs data visualized using the Integrated Genomic Viewers software. Three bladder Urothelial Carcinoma harbor FGFR3-TACC3 gene fusions (black box). Red indicates amplification (A), blue indicates deletion (D).

FIGS. 32-1, 32-2, and 32-3 are a graphical representation of segmented CNVs data visualized using the Integrated Genomic Viewers software. One Breast Carcinoma harbors FGFR3-TACC3 gene fusions (black box). Red indicates amplification (A), blue indicates deletion (D).

FIGS. 33-1, 33-2, and 33-3 are a graphical representation of segmented CNVs data visualized using the Integrated Genomic Viewers software. One Colorectal Carcinoma harbors FGFR3-TACC3 gene fusions (black box). Red indicates amplification (A), blue indicates deletion (D).

FIGS. 34-1, 34-2, and 34-3 are a graphical representation of segmented CNVs data visualized using the Integrated Genomic Viewers software. One Lung Squamous Cell Carcinoma harbors FGFR3-TACC3 gene fusions (black box). Red indicates amplification (A), blue indicates deletion (D).

FIGS. 35-1, 35-2, and 35-3 are a graphical representation of segmented CNVs data visualized using the Integrated Genomic Viewers software. One Head abd Neck Squamous Cell Carcinoma harbors FGFR3-TACC3 gene fusions (black box). Red indicates amplification (A), blue indicates deletion (D).

FIG. 36 shows the structure of FGFR-TACC gene fusions identified by RT-PCR-Sanger sequencing (see also SEQ ID NOs: 530-547). Predicted FGFR-TACC fusion proteins encoded by the transcripts identified by RT-PCR. Regions corresponding to FGFR3 or TACC3 are shown in red or blue, respectively. FGFR1 and TACC1 corresponding regions are shown in yellow and green. On the left are indicated the FGFR and TACC exons joined in the fused mRNA; the presence of TACC3 introns is also reported when they are spliced in the fusion cDNA. On the right, the number of patients harboring the corresponding fusion variant is indicated. The novel transcripts discovered in this study are highlighted in red. Black arrows indicate the position of the primers used for the FGFR-TACC fusions screening.

FIGS. 37A-H show the identification and immunostaining of FGFR3-TACC3-positive tumors. Results from RT-PCR screening in representative samples from the Pitié-Salpêtrière Hospital (A, C) and the Besta (B, D) datasets. M, DNA ladder. Schematic representation of the FGFR3-TACC3 fusion transcripts identified in samples GBM-4620 (C) and GBM-021 (D). The junction sequences on the mRNA (GBM-4620 (C) SEQ ID NO: 515; GBM-021 (D) SEQ ID NO: 517) and the reading frame and translation (GBM-4620 (C) SEQ ID NO: 516; GBM-021 (D) SEQ ID NO: 518) at the breakpoint are reported. Representative microphotographs of H&E and FGFR3 immunostaining in the FGFR3-TACC3 positive samples GBM-4620 (E) and GBM-021 (F) and two FGFR3-TACC3 negative samples (panels G and H); a, H&E, 10× magnification; b, H&E, 40× magnification; c, FGFR3, 10× magnification; d, FGFR3, 40× magnification. FIGS. 37C and 37D disclose chromatogram readings as SEQ ID NOs: 550 and 551, respectively.

FIGS. 38A-D show pre-clinical evaluation of FGFR3-TACC3 inhibition by JNJ-42756493. (A) Mouse astrocytes expressing FGFR3-TACC3 (F3T3), FGFR3-TACC3-KD (F3T3-KD) or the empty vector (Vector) were treated with the indicated concentration of JNJ-42756493. Cell viability was determined by the MTT assay. Error bars show mean±SEM (n=6). (B) Survival analysis of GIC28 1123 treated with JNJ-42756493. (C) The FGFR-TK inhibitor JNJ-42756493 suppresses tumor growth of subcutaneous tumors generated by GIC-1123. After tumor establishment (arrow) mice were treated with vehicle or JNJ-42756493 (12 mg/kg) for 14 days. Values are mean tumor volumes±SD, (n=9 mice per group). P-value of the slope calculated from the treatment starting point (arrow) is 0.04. (D) Photograph showing the tumors dissected from vehicle or JNJ-42756493 treated mice after two weeks of treatment.

FIGS. 39A-G show baseline and post-treatment Magnetic Resonance Imaging (MRI) of patients treated with JNJ-42756493. Patient 1 (Panels A-D). (A) Post-gadolinium T1 weighted images show the target lesion on the right parietal lobe. The interval (days) from the beginning of follow-up is indicated above each MRI. (B) Analysis of sum of product diameters (SPD) before and during the anti-FGFR treatment (RANO criteria). (C) Analysis of tumor volume (cm3) before and during the anti-FGFR treatment. During anti-FGFR treatment a stabilization of the tumor was observed according to RANO criteria and volumetry. (D) Perfusion images at baseline and after 20 days of anti-FGFR treatment. rCBV (relative cerebral blood volume). Post-gadolinium T1 weighted images with color overlay of rCBV are shown. Patient 2 (Panels E-G). (E) Two different MRI slice levels of superior and middle part of the lesion are presented. (F) Analysis of sum of product diameters (SPD) before and during the anti-FGFR treatment. During the anti-FGFR treatment a reduction of 22% of tumor size was observed. (G) Volumetric evaluation showed a 28% tumor reduction. Vertical red arrow indicates the start of anti-FGFR treatment (baseline).

FIG. 40 shows the genomic PCR images and Sanger sequences of FGFR3-TACC3 genomic breakpoints. Fusion specific PCR products and Sanger sequencing chromatograms showing the FGFR3-TACC3 genomic breakpoints (Sample #4451 SEQ ID NO:519; Sample #OPK-14 SEQ ID NO: 520; Sample #MB-22 SEQ ID NO: 521; Sample #3048 SEQ ID NO: 522; Sample #4373 SEQ ID NO: 523; Sample #4867 SEQ ID NO: 524; Sample #3808 SEQ ID NO: 525; Sample #27-1835 SEQ ID NO: 526; Sample #06-6390 SEQ ID NO: 527). The genomic sequences corresponding to FGFR3 and TACC3 are indicated in red or blue, respectively. M, DNA ladder; C—, Negative Control. FIG. 40 discloses chromatogram readings as SEQ ID NOS: 552-560, respectively, in order of appearance.

FIG. 41 shows schematics of FGFR3-TACC3 genomic breakpoints. Schematic representation of the genomic fusions between FGFR3 and TACC3 compared to the corresponding mRNA. In red and blue are reported the regions belonging to FGFR3 and TACC3, respectively. The genomic breakpoint coordinates, according to the genome build GRCh37/hg19, are indicated above each fusion gene.

FIGS. 42A-B show evaluation of the expression of FGFR3-TACC3 fusion elements. (A) Microphotographs of immunofluorescence staining of a representative GBM harboring FGFR3-TACC3 fusion using antibodies that recognize the N- and C-termini of FGFR3 (FGFR3-N, FGFR3-C) and TACC3 (TACC3-N, TACC3-C), red. Nuclei are counterstained with DAPI, blue. (B) Quantitative RT-PCR of four representative GBM carrying FGFR3-TACC3 fusion and three negative controls using primer pairs that amplify FGFR3 and TACC3 regions included in or excluded from the fusion transcripts, as indicated in the diagram. OAW28: ovarian cystoadenocarcinoma cell line harboring wild type FGFR3 and TACC3 genes; GBM55 and GBM0822: GBM harboring wild type FGFR3 and TACC3 genes; GBM3808; GBM1133; GBM0826; GBM3048: GBM harboring FGFR3-TACC3 (F3-T3) fusion. Error bars are SD of triplicate samples.

FIGS. 43A-C show the FGFR3-TACC3 fusion gene and protein are retained in recurrent GBM. (A) FGFR3-TACC3 fusion specific RT-PCR product from untreated and recurrent GBM from patient #3124. (B) Sanger sequencing chromatogram showing the identical reading frame at the breakpoint (SEQ ID NO: 517) and the putative translation of the fusion protein (SEQ ID NO: 86) in the untreated and recurrent tumor from the same patient. The fused exons at mRNA level are shown. Regions corresponding to FGFR3 and TACC3 are indicated in red and blue, respectively. T, threonine; S, serine; D, aspartic acid; V, valine; K, lysine; A, alanine. (C) Representative microphotographs of FGFR3 immunofluorescence (IF) staining in both untreated and recurrent GBM. Blue staining, DAPI; Red staining, FGFR3. 10× Magnification. FIG. 43B discloses chromatogram readings as SEQ ID NOs: 551 and 551, respectively, in order of appearance.

FIGS. 44A-B show PFS and OS of FGFR3-TACC3-positive glioma patients. (A) Kaplan-Meier curves in IDH wild-type glioma patients don't show significant differences in Progression Free Survival (PFS) between FGFR3-TACC3 positive (N=12, median PFS=11.20 months) and FGFR3-TACC3 negative (N=274, Median PFS=12.27 months) (P=0.85). (B) Kaplan-Meier curves in IDH wild-type glioma patients don't show significant differences in Overall Survival (OS) between FGFR3-TACC3 positive (N=12, Median OS=32.80 months) and FGFR3-TACC3 negative (N=326, Median OS=18.60 months) (P=0.6). In red FGFR3-TACC3 positive patients, in green FGFR3-TACC3 negative patients. Open circles represent censored patients.

FIG. 45 shows analysis of SNP6.0 arrays of GBM harboring CNVs of FGFR3 and TACC3 genomic loci. CNVs of the FGFR3/TACC3 genomic loci in “gain labeled” (LRR>0.2) TCGA samples. The CNA magnitudes (expressed as log 2 ratio) were classified using simple thresholds: deletion (x<−1), loss (−1<x≤−0.2), gain (0.2≤x<1) or amplification (x>1). Gains are in gradient of red, loss in gradient of blue. Samples with uniform gains/amplification of FGFR3 and TACC3 lack FGFR3-TACC3 fusions. Samples harboring FGFR3-TACC3 fusions (F3-T3) show microamplifications involving the first FGFR3 exons, which are spliced in the fusion gene.

### DETAILED DESCRIPTION OF THE INVENTION

Glioblastoma multiformes (GBMs) are the most common form of brain tumors in adults accounting for 12-15% of intracranial tumors and 50-60% of primary brain tumors. GBM is among the most lethal forms of human cancer. The history of successful targeted therapy of cancer largely coincides with the inactivation of recurrent and oncogenic gene fusions in hematological malignancies and recently in some types of epithelial cancer. GBM is among the most lethal and incurable forms of human cancer. Targeted therapies against common genetic alterations in GBM have not changed the dismal clinical outcome of the disease, most likely because they have systematically failed to eradicate the truly addicting oncoprotein activities of GBM. Recurrent chromosomal rearrangements resulting in the creation of oncogenic gene fusions have not been found in GBM.

GBM is among the most difficult forms of cancer to treat in humans (1). So far, the therapeutic approaches that have been tested against potentially important oncogenic targets in GBM have met limited success (2-4). Recurrent chromosomal translocations leading to production of oncogenic fusion proteins are viewed as initiating and addicting events in the pathogenesis of human cancer, thus providing the most desirable molecular targets for cancer therapy (5, 6). Recurrent and oncogenic gene fusions have not been found in GBM. Chromosomal rearrangements are hallmarks of hematological malignancies but recently they have also been uncovered in subsets of solid tumors (breast, prostate, lung and colorectal carcinoma) (7, 8). Important and successful targeted therapeutic interventions for patients whose tumors carry these rearrangements have stemmed from the discovery of functional gene fusions, especially when the translocations involve kinase-coding genes (BCR-ABL, EML4-ALK) (9, 10).

A hallmark of GBM is rampant chromosomal instability (CIN), which leads to aneuploidy (11). CIN and aneuploidy are early events in the pathogenesis of cancer (12). It has been suggested that genetic alterations targeting mitotic fidelity might be responsible for missegregation of chromosomes during mitosis, resulting in aneuploidy (13, 14).

Fibroblast growth factor receptors (FGFR) are transmembrane receptors that bind to members of the fibroblast growth factor family of proteins. The structure of the FGFRs consist of an extracellular ligand binding domain comprised of three Ig-like domains, a single transmembrane helix domain, and an intracellular domain with tyrosine kinase activity (Johnson, D. E., Williams, E. T. Structural and functional diversity in the FGF receptor multigene family. (1993) Adv. Cancer Res, 60:1-41).

Transforming acidic coiled-coiled protein (TACC) stabilize microtubules during mitosis by recruiting minispindles (Msps)/XMAP215 proteins to centrosomes. TACCs have been implicated in cancer.

From a medical perspective, the FGFR-TACC fusions provide the first “bona-fide” oncogenically addictive gene fusions in GBM whose identification has long been overdue in this disease.

Beside GBM, which features the highest grade of malignancy among glioma (grade IV), lower grade glioma which include grade II and grade III are a heterogeneous group of tumors in which specific molecular features are associated with divergent clinical outcome. The majority of grade II-III glioma (but only a small subgroup of GBM) harbor mutations in IDH genes (IDH1 or IDH2), which confer a more favorable clinical outcome. Conversely, the absence of IDH mutations is associated with the worst prognosis (5).

Described herein is the identification of FGFR-TACC gene fusions (mostly FGFR3-TACC3, and rarely FGFR1-TACC1) as the first example of highly oncogenic and recurrent gene fusions in GBM. The FGFR-TACC fusions that have been identified so far include the Tyrosine Kinase (TK) domain of FGFR and the coiled-coil domain of TACC proteins, both necessary for the oncogenic function of FGFR-TACC fusions. FGFR3-TACC3 fusions have been identified in pediatric and adult glioma, bladder carcinoma, squamous lung carcinoma and head and neck carcinoma, thus establishing FGFR-TACC fusions as one of the chromosomal translocation most frequently found across multiple types of human cancers (6-15).

Here a screening method for FGFR-TACC fusions is reported that includes a RT-PCR assay designed to identify the known and novel FGFR3-TACC3 fusion transcripts, followed by confirmation of the inframe breakpoint by Sanger sequencing. Using this assay, a dataset of 584 GBM and 211 grade II and grade III gliomas has been analyzed. It was determined that brain tumors harboring FGFR-TACC fusions manifest strong and homogeneous intra-tumor expression of the FGFR3 and TACC3 component invariably included in the fusion protein, when analyzed by immunostaining. A significant clinical benefit following treatment with a specific inhibitor of FGFR-TK is reported in two GBM patients who harbored FGFR3-TACC3 rearrangement.

### DNA and AminoAcid Manipulation Methods and Purification Thereof

The practice of aspects of the present invention can employ, unless otherwise indicated, conventional techniques of cell biology, cell culture, molecular biology, transgenic biology, microbiology, recombinant DNA, and immunology, which are within the skill of the art. Such techniques are explained fully in the literature. See, e.g., Molecular Cloning A Laboratory Manual, 3^{rd} Ed., ed. by Sambrook (2001), Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription and Translation (B. D. Hames & S. J. Higgins eds. 1984); Culture Of Animal Cells (R. I. Freshney, Alan R. Liss, Inc., 1987); Immobilized Cells and Enzymes (IRL Press, 1986); B. Perbal, A Practical Guide To Molecular Cloning (1984); the series, Methods In Enzymology (Academic Press, Inc., N.Y.), specifically, Methods In Enzymology, Vols. 154 and 155 (Wu et al. eds.); Gene Transfer Vectors For Mammalian Cells (J. H. Miller and M. P. Calos eds., 1987, Cold Spring Harbor Laboratory); Immunochemical Methods In Cell And Molecular Biology (Caner and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986). All patents, patent applications and references cited herein are incorporated by reference in their entireties.

One skilled in the art can obtain a protein in several ways, which include, but are not limited to, isolating the protein via biochemical means or expressing a nucleotide sequence encoding the protein of interest by genetic engineering methods.

A protein is encoded by a nucleic acid (including, for example, genomic DNA, complementary DNA (cDNA), synthetic DNA, as well as any form of corresponding RNA). For example, it can be encoded by a recombinant nucleic acid of a gene. The proteins of the invention can be obtained from various sources and can be produced according to various techniques known in the art. For example, a nucleic acid that encodes a protein can be obtained by screening DNA libraries, or by amplification from a natural source. A protein can be a fragment or portion thereof. The nucleic acids encoding a protein can be produced via recombinant DNA technology and such recombinant nucleic acids can be prepared by conventional techniques, including chemical synthesis, genetic engineering, enzymatic techniques, or a combination thereof. For example, a fusion protein of the invention comprises a tyrosine kinase domain of an FGFR protein fused to a polypeptide that constitutively activates the tyrosine kinase domain of the FGFR protein. For example, a fusion protein of the invention comprises a transforming acidic coiled-coil (TACC) domain fused to a polypeptide with a tyrosine kinase domain, wherein the TACC domain constitutively activates the tyrosine kinase domain. An example of a FGFR1-TACC1 polypeptide has the amino acid sequence shown in SEQ ID NO: 150. An example of a FGFR3-TACC3 protein is the polypeptide encoded by the nucleic acid having the nucleotide sequence shown in SEQ ID NOs: 94, 530, 531, 532, 533, 534, 535, 536, 537, or 538. Examples of a FGFR3-TACC3 polypeptide has the amino acid sequence shown in SEQ ID NO: 79, 158, 159, 160, 161, 539, 540, 541, 542, 543, 544, 545, 546, or 547.

The Genbank ID for the FGFR3 gene is 2261. Three isoforms are listed for FGFGR3, e.g., having Genebank Accession Nos. NP_000133 (corresponding nucleotide sequence NM_000142); NP_001156685 (corresponding nucleotide sequence NM_001163213); NP_075254 (corresponding nucleotide sequence NM_022965).

SEQ ID NO: 90 is the FGFR3 Amino Acid Sequence, Transcript Variant 1 (NP_000133; 806 aa). The location of exons are marked by alternating underlining. Amino acids encoded by nucleotides spanning exons are bold italicized.

| | |
|---|---|
| 1 | MGAPACALAL CVAVAIVAGA SSESLGTEQR VVGRAAEVPG PEPGQQEQLV FGSGDAVELS |
| 61 | CPPPGGGPMG PTVWVKDGTG LVPSERVLVG PQRLQVLNAS HEDSGAYSCR QRLTQRVLCH |
| 121 | FSVRVTDAPS SGDDEDGEDE AEDTGVDTGA PYWTRPERMD KKLLAVPAAN TVRFRCPAAG |
| 181 | NPTPSISWLK NGREFRGEHR IGGIKLRHQQ WSLVMESVVP SDRGNYTCVV ENKFGSIRQT |
| 241 | YTLDVLERSP HRPILQAGLP ANQTAVLGSD VEFHCKVYSD AQPHIQWLKH VEVNGSKVGP |
| 301 | DGTPYVTVLK TAGANTTDKE LEVLSLHNVT FEDAGEYTCL AGNSIGFSHH SAWLVVLPAE |
| 361 | EELVEADEAG SVYAGILSYG VGFFLFILVV AAVTLCRLRS PPKKGLGSPT VHKISRFPLK |
| 421 | RQVSLESNAS MSSNTPLVRI ARLSSGEGPT LANVSELELP ADPKWELSRA RLTLGKPLGE |
| 481 | GCFGQVVMAE AIGIDKDRAA KPVTVAVKML KDDATDKDLS DLVSEMEMMK MIGKHKNIIN |
| 541 | LLGACTQGGP LYVLVEYAAK GNLREFLRAR RPPGLDYSFD TCKPPEEQLT FKDLVSCAYQ |
| 601 | VARGMEYLAS QKCIHRDLAA RNVLVTEDNV MKIADFGLAR DVHNLDYYKK TTNGRLPVKW |
| 661 | MAPEALFDRV YTHQSDVWSF GVLLWEIFTL GGSPYPGIPV EELFKLLKEG HRMDKPANCT |
| 721 | HDLYMIMREC WHAAPSQRPT FKQLVEDLDR VLTVTSTDEY LDLSAPFEQY SPGGQDTPSS |
| 781 | SSSGDDSVFA HDLLPPAPPS SGGSRT |

SEQ ID NO: 91 is the FGFR3 Nucleotide Sequence, Transcript Variant 1 (NM_000142; 4304 bp).

| | |
|---|---|
| 1 | gtcgcgggca gctggcgccg cgcggtcctg ctctgccggt cgcacggacg caccggcggg |
| 61 | ccgccggccg gagggacggg gcgggagctg ggcccgcgga cagcgagccg gagcgggagc |
| 121 | cgcgcgtagc gagccgggct ccggcgctcg ccagtctccc gagcggcgcc cgcctcccgc |
| 181 | cggtgcccgc gccgggccgt ggggggcagc atgcccgcgc gcgctgcctg aggacgccgc |
| 241 | ggcccccgcc cccgccatgg gcgcccctgc ctgcgccctc gcgctctgcg tggccgtggc |
| 301 | catcgtggcc ggcgcctcct cggagtcctt ggggacggag cagcgcgtcg tggggcgagc |
| 361 | ggcagaagtc ccgggcccag agcccggcca gcaggagcag ttggtcttcg gcagcgggga |
| 421 | tgctgtggag ctgagctgtc ccccgcccgg gggtggtccc atggggccca ctgtctgggt |
| 481 | caaggatggc acagggctgg tgccctcgga gcgtgtcctg gtggggcccc agcggctgca |
| 541 | ggtgctgaat gcctcccacg aggactccgg ggcctacagc tgccggcagc ggctcacgca |
| 601 | gcgcgtactg tgccacttca gtgtgcgggt gacagacgct ccatcctcgg gagatgacga |
| 661 | agacggggag gacgaggctg aggacacagg tgtggacaca ggggcccctt actggacacg |
| 721 | gcccgagcgg atggacaaga agctgctggc cgtgccggcc gccaacaccg tccgcttccg |
| 781 | ctgcccagcc gctggcaacc ccactccctc catctcctgg ctgaagaacg gcagggagtt |
| 841 | ccgcggcgag caccgcattg gaggcatcaa gctgcggcat cagcagtgga gcctggtcat |
| 901 | ggaaagcgtg gtgccctcgg accgcggcaa ctacacctgc gtcgtggaga acaagtttgg |
| 961 | cagcatccgg cagacgtaca cgctggacgt gctggagcgc tccccgcacc ggcccatcct |
| 1021 | gcaggcgggg ctgccggcca accagacggc ggtgctgggc agcgacgtgg agttccactg |
| 1081 | caaggtgtac agtgacgcac agccccacat ccagtggctc aagcacgtgg aggtgaatgg |
| 1141 | cagcaaggtg ggcccggacg gcacacccta cgttaccgtg ctcaagacgg cgggcgctaa |
| 1201 | caccaccgac aaggagctag aggttctctc cttgcacaac gtcacctttg aggacgccgg |
| 1261 | ggagtacacc tgcctggcgg gcaattctat tgggttttct catcactctg cgtggctggt |
| 1321 | ggtgctgcca gccgaggagg agctggtgga ggctgacgag gcgggcagtg tgtatgcagg |
| 1381 | catcctcagc tacggggtgg gcttcttcct gttcatcctg gtggtggcgg ctgtgacgct |
| 1441 | ctgccgcctg cgcagccccc ccaagaaagg cctgggctcc cccaccgtgc acaagatctc |
| 1501 | ccgcttcccg ctcaagcgac aggtgtccct ggagtccaac gcgtccatga gctccaacac |
| 1561 | accactggtg cgcatcgcaa ggctgtcctc aggggagggc cccacgctgg ccaatgtctc |
| 1621 | cgagctcgag ctgcctgccg accccaaatg ggagctgtct cgggcccggc tgaccctggg |
| 1681 | caagcccctt ggggagggct gcttcggcca ggtggtcatg gcggaggcca tcggcattga |
| 1741 | caaggaccgg gccgccaagc ctgtcaccgt agccgtgaag atgctgaaag acgatgccac |
| 1801 | tgacaaggac ctgtcggacc tggtgtctga gatggagatg atgaagatga tcgggaaaca |
| 1861 | caaaaacatc atcaacctgc tgggcgcctg cacgcagggc gggcccctgt acgtgctggt |
| 1921 | ggagtacgcg gccaagggta acctgcggga gtttctgcgg gcgcggcggc ccccgggcct |
| 1981 | ggactactcc ttcgacacct gcaagccgcc cgaggagcag ctcaccttca aggacctggt |
| 2041 | gtcctgtgcc taccaggtgg cccggggcat ggagtacttg gcctcccaga agtgcatcca |
| 2101 | cagggacctg gctgcccgca atgtgctggt gaccgaggac aacgtgatga agatcgcaga |
| 2161 | cttcgggctg gcccgggacg tgcacaacct cgactactac aagaagacaa ccaacggccg |
| 2221 | gctgcccgtg aagtggatgg cgcctgaggc cttgtttgac cgagtctaca ctcaccagag |
| 2281 | tgacgtctgg tcctttgggg tcctgctctg ggagatcttc acgctggggg gctccccgta |
| 2341 | ccccggcatc cctgtggagg agctcttcaa gctgctgaag gagggccacc gcatggacaa |
| 2401 | gcccgccaac tgcacacacg acctgtacat gatcatgcgg gagtgctggc atgccgcgcc |
| 2461 | ctcccagagg cccaccttca agcagctggt ggaggacctg gaccgtgtcc ttaccgtgac |
| 2521 | gtccaccgac gagtacctgg acctgtcggc gcctttcgag cagtactccc cgggtggcca |
| 2581 | ggacaccccc agctccagct cctcagggga cgactccgtg tttgcccacg acctgctgcc |
| 2641 | cccggcccca cccagcagtg ggggctcgcg gacgtgaagg gccactggtc cccaacaatg |
| 2701 | tgaggggtcc ctagcagccc accctgctgc tggtgcacag ccactccccg gcatgagact |
| 2761 | cagtgcagat ggagagacag ctacacagag ctttggtctg tgtgtgtgtg tgtgcgtgtg |
| 2821 | tgtgtgtgtg tgtgcacatc cgcgtgtgcc tgtgtgcgtg cgcatcttgc ctccaggtgc |
| 2881 | agaggtaccc tgggtgtccc cgctgctgtg caacggtctc ctgactggtg ctgcagcacc |
| 2941 | gaggggcctt tgttctgggg ggacccagtg cagaatgtaa gtgggcccac ccggtgggac |
| 3001 | ccccgtgggg cagggagctg ggcccgacat ggctccggcc tctgcctttg caccacggga |
| 3061 | catcacaggg tgggcctcgg cccctcccac acccaaagct gagcctgcag ggaagcccca |
| 3121 | catgtccagc accttgtgcc tggggtgtta gtggcaccgc ctccccacct ccaggctttc |
| 3181 | ccacttccca ccctgcccct cagagactga aattacgggt acctgaagat gggagccttt |
| 3241 | accttttatg caaaaggttt attccggaaa ctagtgtaca tttctataaa tagatgctgt |
| 3301 | gtatatggta tatatacata tatatatata acatatatgg aagaggaaaa ggctggtaca |
| 3361 | acggaggcct gcgaccctgg gggcacagga ggcaggcatg gccctgggcg gggcgtgggg |
| 3421 | gggcgtggag ggaggcccca gggggtctca cccatgcaag cagaggacca gggccttttc |
| 3481 | tggcaccgca gttttgtttt aaaactggac ctgtatattt gtaaagctat ttatgggccc |
| 3541 | ctggcactct tgttcccaca ccccaacact tccagcattt agctggccac atggcggaga |
| 3601 | gttttaattt ttaacttatt gacaaccgag aaggtttatc ccgccgatag agggacggcc |
| 3661 | aagaatgtac gtccagcctg ccccggagct ggaggatccc ctccaagcct aaaaggttgt |
| 3721 | taatagttgg aggtgattcc agtgaagata ttttatttcc tttgtccttt ttcaggagaa |
| 3781 | ttagatttct ataggatttt tctttaggag atttattttt tggacttcaa agcaagctgg |
| 3841 | tattttcata caaattcttc taattgctgt gtgtcccagg cagggagacg gtttccaggg |
| 3901 | aggggccggc cctgtgtgca ggttccgatg ttattagatg ttacaagttt atatatatct |
| 3961 | atatatataa tttattgagt ttttacaaga tgtatttgtt gtagacttaa cacttcttac |
| 4021 | gcaatgcttc tagagtttta tagcctggac tgctaccttt caaagcttgg agggaagccg |
| 4081 | tgaattcagt tggttcgttc tgtactgtta ctgggccctg agtctgggca gctgtccctt |
| 4141 | gcttgcctgc agggccatgg ctcagggtgg tctcttcttg gggcccagtg catggtggcc |
| 4201 | agaggtgtca cccaaaccgg caggtgcgat tttgttaacc cagcgacgaa ctttccgaaa |
| 4261 | aataaagaca cctggttgct aacctggaaa aaaaaaaaaa aaaa |

SEQ ID NO: 528 is the FGFR3 wt cDNA Nucleotide Sequence corresponding to the coding sequence of FGFR3 (2421 bp) (NM_000142.4 NP_000133.1). The location of exons are marked by alternating underlining.

ATGGGCGCCCCTGCCTGCGCCCTCGCGCTCTGCGTGGCCGTGGCCATCGTGGCCGGCGCCTCCTCGGAGT
CCTTGGGGACGGAGCAGCGCGTCGTGGGGCGAGCGGCAGAAGTCCCGGGCCCAGAGCCCGGCCAGCAGGA
GCAGTTGGTCTTCGGCAGCGGGGATGCTGTGGAGCTGAGCTGTCCCCCGCCCGGGGGTGGTCCCATGGGG
CCCACTGTCTGGGTCAAGGATGGCACAGGGCTGGTGCCCTCGGAGCGTGTCCTGGTGGGGCCCCAGCGGC
TGCAGGTGCTGAATGCCTCCCACGAGGACTCCGGGGCCTACAGCTGCCGGCAGCGGCTCACGCAGCGCGT
ACTGTGCCACTTCAGTGTGCGGGTGACAGACGCTCCATCCTCGGGAGATGACGAAGACGGGGAGGACGAG
GCTGAGGACACAGGTGTGGACACAGGGGCCCCTTACTGGACACGGCCCGAGCGGATGGACAAGAAGCTGC
TGGCCGTGCCGGCCGCCAACACCGTCCGCTTCCGCTGCCCAGCCGCTGGCAACCCCACTCCCTCCATCTC
CTGGCTGAAGAACGGCAGGGAGTTCCGCGGCGAGCACCGCATTGGAGGCATCAAGCTGCGGCATCAGCAG
TGGAGCCTGGTCATGGAAAGCGTGGTGCCCTCGGACCGCGGCAACTACACCTGCGTCGTGGAGAACAAGT
TTGGCAGCATCCGGCAGACGTACACGCTGGACGTGCTGGAGCGCTCCCCGCACCGGCCCATCCTGCAGGC
GGGGCTGCCGGCCAACCAGACGGCGGTGCTGGGCAGCGACGTGGAGTTCCACTGCAAGGTGTACAGTGAC
GCACAGCCCCACATCCAGTGGCTCAAGCACGTGGAGGTGAATGGCAGCAAGGTGGGCCCGGACGGCACAC
CCTACGTTACCGTGCTCAAGACGGCGGGCGCTAACACCACCGACAAGGAGCTAGAGGTTCTCTCCTTGCA
CAACGTCACCTTTGAGGACGCCGGGGAGTACACCTGCCTGGCGGGCAATTCTATTGGGTTTTCTCATCAC
TCTGCGTGGCTGGTGGTGCTGCCAGCCGAGGAGGAGCTGGTGGAGGCTGACGAGGCGGGCAGTGTGTATG
CAGGCATCCTCAGCTACGGGGTGGGCTTCTTCCTGTTCATCCTGGTGGTGGCGGCTGTGACGCTCTGCCG
CCTGCGCAGCCCCCCCAAGAAAGGCCTGGGCTCCCCCACCGTGCACAAGATCTCCCGCTTCCCGCTCAAG
CGACAGGTGTCCCTGGAGTCCAACGCGTCCATGAGCTCCAACACACCACTGGTGCGCATCGCAAGGCTGT
CCTCAGGGGAGGGCCCCACGCTGGCCAATGTCTCCGAGCTCGAGCTGCCTGCCGACCCCAAATGGGAGCT
GTCTCGGGCCCGGCTGACCCTGGGCAAGCCCCTTGGGGAGGGCTGCTTCGGCCAGGTGGTCATGGCGGAG
GCCATCGGCATTGACAAGGACCGGGCCGCCAAGCCTGTCACCGTAGCCGTGAAGATGCTGAAAGACGATG
CCACTGACAAGGACCTGTCGGACCTGGTGTCTGAGATGGAGATGATGAAGATGATCGGGAAACACAAAAA
CATCATCAACCTGCTGGGCGCCTGCACGCAGGGCGGGCCCCTGTACGTGCTGGTGGAGTACGCGGCCAAG
GGTAACCTGCGGGAGTTTCTGCGGGCGCGGCGGCCCCCGGGCCTGGACTACTCCTTCGACACCTGCAAGC
CGCCCGAGGAGCAGCTCACCTTCAAGGACCTGGTGTCCTGTGCCTACCAGGTGGCCCGGGGCATGGAGTA
CTTGGCCTCCCAGAAGTGCATCCACAGGGACCTGGCTGCCCGCAATGTGCTGGTGACCGAGGACAACGTG
ATGAAGATCGCAGACTTCGGGCTGGCCCGGGACGTGCACAACCTCGACTACTACAAGAAGACGACCAACG
GCCGGCTGCCCGTGAAGTGGATGGCGCCTGAGGCCTTGTTTGACCGAGTCTACACTCACCAGAGTGACGT
CTGGTCCTTTGGGGTCCTGCTCTGGGAGATCTTCACGCTGGGGGGCTCCCCGTACCCCGGCATCCCTGTG
GAGGAGCTCTTCAAGCTGCTGAAGGAGGGCCACCGCATGGACAAGCCCGCCAACTGCACACACGACCTGT
ACATGATCATGCGGGAGTGCTGGCATGCCGCGCCCTCCCAGAGGCCCACCTTCAAGCAGCTGGTGGAGGA
CCTGGACCGTGTCCTTACCGTGACGTCCACCGACGAGTACCTGGACCTGTCGGCGCCTTTCGAGCAGTAC
TCCCCGGGTGGCCAGGACACCCCCAGCTCCAGCTCCTCAGGGGACGACTCCGTGTTTGCCCACGACCTGC
TGCCCCCGGCCCCACCCAGCAGTGGGGGCTCGCGGACGTGA

The Genbank ID for the TACC3 gene is 10460. SEQ ID NO: 92 is the TACC3 Amino Acid Sequence (NP_006333) (838 aa). The location of exons are marked by alternating underlining. Amino acids encoded by nucleotides spanning exons are shaded in gray. Double underlining indicates the amino acid encoded by bold italicized the nucleotides in SEQ ID NO: 529.

| | |
|---|---|
| 1 | MSLQVLNDKN VSNEKNTENC DFLFSPPEVT GRSSVLRVSQ KENVPPKNLA KAMKVTFQTP |
| 61 | LRDPQTHRIL SPSMASKLEA PFTQDDTLGL ENSHPVWTQK ENQQLIKEVD AKTTHGILQK |
| 121 | PVEADTDLLG DASPAFGSGS SSESGPGALA DLDCSSSSQS PGSSENQMVS PGKVSGSPEQ |
| 181 | AVEENLSSYS LDRRVTPASE TLEDPCRTES QHKAETPHGA EEECKAETPH GAEEECRHGG |
| 241 | VCAPAAVATS PPGAIPKEAC GGAPLQGLPG EALGCPAGVG TPVPADGTQT LTCAHTSAPE |
| 301 | STAPTNHLVA GRAMTLSPQE EVAAGQMASS SRSGPVKLEF DVSDGATSKR APPPRRLGER |
| 361 | SGLKPPLRKA AVRQQKAPQE VEEDDGRSGA GEDPPMPASR GSYHLDWDKM DDPNFIPFGG |
| 421 | DTKSGCSEAQ PPESPETRLG QPAAEQLHAG PATEEPGPCL SQQLHSASAE DTPVVQLAAE |
| 481 | TPTAESKERA LNSASTSLPT SCPGSEPVPT HQQGQPALEL KEESFRDPAE VLGTGAEVDY |
| 541 | LEQFGTSSFK ESALRKQSLY LKFDPLLRDS PGRPVPVATE TSSMHGANET PSGRPREAKL |
| 601 | VEFDFLGALD IPVPGPPPGV PAPGGPPLST GPIVDLLQYS QKDLDAVVKA TQEENRELRS |
| 661 | RCEELHGKNL ELGKIMDRFE EVVYQAMEEV QKQKELSKAE IQKVLKEKDQ LTTDLNSMEK |
| 721 | SFSDLFKRFE KQKEVIEGYR KNEESLKKCV EDYLARITQE GQRYQALKAH AEEKLQLANE |
| 781 | EIAQVRSKAQ AEALALQASL RKEQMRIQSL EKTVEQKTKE NEELTRICDD LISKMEKI |

SEQ ID NO: 93 is the TACC3 Nucleotide Sequence (NM_006342) (2847 bp):

| | |
|---|---|
| 1 | gcgtttgaaa ctccggcgcg ccggcggcca tcaagggcta gaagcgcgac ggcggtagca |
| 61 | gctaggcttg gcccccggcg tggagcagac gcggacccct ccttcctggc ggcggcggcg |
| 121 | cgggctcaga gcccggcaac gggcgggcgg gcagaatgag tctgcaggtc ttaaacgaca |
| 181 | aaaatgtcag caatgaaaaa aatacagaaa attgcgactt cctgttttcg ccaccagaag |
| 241 | ttaccggaag atcgtctgtt cttcgtgtgt cacagaaaga aaatgtgcca cccaagaacc |
| 301 | tggccaaagc tatgaaggtg acttttcaga cacctctgcg ggatccacag acgcacagga |
| 361 | ttctaagtcc tagcatggcc agcaaacttg aggctccttt cactcaggat gacacccttg |
| 421 | gactggaaaa ctcacacccg gtctggacac agaaagagaa ccaacagctc atcaaggaag |
| 481 | tggatgccaa aactactcat ggaattctac agaaaccagt ggaggctgac accgacctcc |
| 541 | tgggggatgc aagcccagcc tttgggagtg gcagctccag cgagtctggc ccaggtgccc |
| 601 | tggctgacct ggactgctca agctcttccc agagcccagg aagttctgag aaccaaatgg |
| 661 | tgtctccagg aaaagtgtct ggcagccctg agcaagccgt ggaggaaaac cttagttcct |
| 721 | attccttaga cagaagagtg acacccgcct ctgagaccct agaagaccct tgcaggacag |
| 781 | agtcccagca caaagcggag actccgcacg gagccgagga agaatgcaaa gcggagactc |
| 841 | cgcacggagc cgaggaggaa tgccggcacg gtggggtctg tgctcccgca gcagtggcca |
| 901 | cttcgcctcc tggtgcaatc cctaaggaag cctgcggagg agcacccctg cagggtctgc |
| 961 | ctggcgaagc cctgggctgc cctgcgggtg tgggcacccc cgtgccagca gatggcactc |
| 1021 | agacccttac ctgtgcacac acctctgctc ctgagagcac agccccaacc aaccacctgg |
| 1081 | tggctggcag ggccatgacc ctgagtcctc aggaagaagt ggctgcaggc caaatggcca |
| 1141 | gctcctcgag gagcggacct gtaaaactag aatttgatgt atctgatggc gccaccagca |
| 1201 | aaagggcacc cccaccaagg agactgggag agaggtccgg cctcaagcct cccttgagga |
| 1261 | aagcagcagt gaggcagcaa aaggccccgc aggaggtgga ggaggacgac ggtaggagcg |
| 1321 | gagcaggaga ggaccccccc atgccagctt ctcggggctc ttaccacctc gactgggaca |
| 1381 | aaatggatga cccaaacttc atcccgttcg gaggtgacac caagtctggt tgcagtgagg |
| 1441 | cccagccccc agaaagccct gagaccaggc tgggccagcc agcggctgaa cagttgcatg |
| 1501 | ctgggcctgc cacggaggag ccaggtccct gtctgagcca gcagctgcat tcagcctcag |
| 1561 | cggaggacac gcctgtggtg cagttggcag ccgagacccc aacagcagag agcaaggaga |
| 1621 | gagccttgaa ctctgccagc acctcgcttc ccacaagctg tccaggcagt gagccagtgc |
| 1681 | ccacccatca gcaggggcag cctgccttgg agctgaaaga ggagagcttc agagaccccg |
| 1741 | ctgaggttct aggcacgggc gcggaggtgg attacctgga gcagtttgga acttcctcgt |
| 1801 | ttaaggagtc ggccttgagg aagcagtcct tatacctcaa gttcgacccc ctcctgaggg |
| 1861 | acagtcctgg tagaccagtg cccgtggcca ccgagaccag cagcatgcac ggtgcaaatg |
| 1921 | agactccctc aggacgtccg cgggaagcca agcttgtgga gttcgatttc ttgggagcac |
| 1981 | tggacattcc tgtgccaggc ccacccccag gtgttcccgc gcctgggggc ccacccctgt |
| 2041 | ccaccggacc tatagtggac ctgctccagt acagccagaa ggacctggat gcagtggtaa |
| 2101 | aggcgacaca ggaggagaac cgggagctga ggagcaggtg tgaggagctc cacgggaaga |
| 2161 | acctggaact ggggaagatc atggacaggt tcgaagaggt tgtgtaccag gccatggagg |
| 2221 | aagttcagaa gcagaaggaa ctttccaaag ctgaaatcca gaaagttcta aaagaaaaag |
| 2281 | accaacttac cacagatctg aactccatgg agaagtcctt ctccgacctc ttcaagcgtt |
| 2341 | ttgagaaaca gaaagaggtg atcgagggct accgcaagaa cgaagagtca ctgaagaagt |
| 2401 | gcgtggagga ttacctggca aggatcaccc aggagggcca gaggtaccaa gccctgaagg |
| 2461 | cccacgcgga ggagaagctg cagctggcaa acgaggagat cgcccaggtc cggagcaagg |
| 2521 | cccaggcgga agcgttggcc ctccaggcca gcctgaggaa ggagcagatg cgcatccagt |
| 2581 | cgctggagaa gacagtggag cagaagacta aagagaacga ggagctgacc aggatctgcg |
| 2641 | acgacctcat ctccaagatg gagaagatct gacctccacg gagccgctgt ccccgccccc |
| 2701 | ctgctcccgt ctgtctgtcc tgtctgattc tcttaggtgt catgttcttt tttctgtctt |
| 2761 | gtcttcaact tttttaaaaa ctagattgct ttgaaaacat gactcaataa aagtttcctt |
| 2821 | tcaatttaaa cactgaaaaa aaaaaaa |

SEQ ID NO: 529 is the TACC3 wt cDNA Nucleotide Sequence corresponding to the coding sequence of TACC3 (2517 bp) (NM_006342.2, NP_006333.1). The location of exons are marked by alternating underlining.

ATGAGTCTGCAGGTCTTAAACGACAAAAATGTCAGCAATGAAAAAAATACAGAAAATTGCGACTTCCTGT
TTTCGCCACCAGAAGTTACCGGAAGATCGTCTGTTCTTCGTGTGTCACAGAAAGAAAATGTGCCACCCAA
GAACCTGGCCAAAGCTATGAAGGTGACTTTTCAGACACCTCTGCGGGATCCACAGACGCACAGGATTCTA
AGTCCTAGCATGGCCAGCAAACTTGAGGCTCCTTTCACTCAGGATGACACCCTTGGACTGGAAAACTCAC
ACCCGGTCTGGACACAGAAAGAGAACCAACAGCTCATCAAGGAAGTGGATGCCAAAACTACTCATGGAAT
TCTACAGAAACCAGTGGAGGCTGACACCGACCTCCTGGGGGATGCAAGCCCAGCCTTTGGGAGTGGCAGC
TCCAGCGAGTCTGGCCCAGGTGCCCTGGCTGACCTGGACTGCTCAAGCTCTTCCCAGAGCCCAGGAAGTT
CTGAGAACCAAATGGTGTCTCCAGGAAAAGTGTCTGGCAGCCCTGAGCAAGCCGTGGAGGAAAACCTTAG
TTCCTATTCCTTAGACAGAAGAGTGACACCCGCCTCTGAGACCCTAGAAGACCCTTGCAGGACAGAGTCC
CAGCACAAAGCGGAGACTCCGCACGGAGCCGAGGAAGAATGCAAAGCGGAGACTCCGCACGGAGCCGAGG
AGGAATGCCGGCACGGTGGGGTCTGTGCTCCCGCAGCAGTGGCCACTTCGCCTCCTGGTGCAATCCCTAA
GGAAGCCTGCGGAGGAGCACCCCTGCAGGGTCTGCCTGGCGAAGCCCTGGGCTGCCCTGCGGGTGTGGGC
ACCCCCGTGCCAGCAGATGGCACTCAGACCCTTACCTGTGCACACACCTCTGCTCCTGAGAGCACAGCCC
CAACCAACCACCTGGTGGCTGGCAGGGCCATGACCCTGAGTCCTCAGGAAGAAGTGGCTGCAGGCCAAAT
GGCCAGCTCCTCGAGGAGCGGACCTGTAAAACTAGAATTTGATGTATCTGATGGCGCCACCAGCAAAAGG
GCACCCCCACCAAGGAGACTGGGAGAGAGGTCCGGCCTCAAGCCTCCCTTGAGGAAAGCAGCAGTGAGGC
AGCAAAAGGCCCCGCAGGAGGTGGAGGAGGACGACGGTAGGAGCGGAGCAGGAGAGGACCCCCCCATGCC
AGCTTCTCGGGGCTCTTACCACCTCGACTGGGACAAAATGGATGACCCAAACTTCATCCCGTTCGGAGGT
GACACCAAGTCTGGTTGCAGTGAGGCCCAGCCCCCAGAAAGCCCTGAGACCAGGCTGGGCCAGCCAGCGG
CTGAACAGTTGCATGCTGGGCCTGCCACGGAGGAGCCAGGTCCCTGTCTGAGCCAGCAGCTGCATTCAGC
CTCAGCGGAGGACACGCCTGTGGTGCAGTTGGCAGCCGAGACCCCAACAGCAGAGAGCAAGGAGAGAGCC
TTGAACTCTGCCAGCACCTCGCTTCCCACAAGCTGTCCAGGCAGTGAGCCAGTGCCCACCCATCAGCAGG
GGCAGCCTGCCTTGGAGCTGAAAGAGGAGAGCTTCAGAGACCCCGCTGAGGTTCTAGGCACGGGCGCGGA
GGTGGATTACCTGGAGCAGTTTGGAACTTCCTCGTTTAAGGAGTCGGCCTTGAGGAAGCAGTCCTTATAC
CTCAAGTTCGACCCCCTCCTGAGGGACAGTCCTGGTAGACCAGTGCCCGTGGCCACCGAGACCAGCAGCA
TGCACGGTGCAAATGAGACTCCCTCAGGACGTCCGCGGGAAGCCAAGCTTGTGGAGTTCGATTTCTTGGG
AGCACTGGACATTCCTGTGCCAGGCCCACCCCCAGGTGTTCCCGCGCCTGGGGGCCCACCCCTGTCCACC
GGACCTATAGTGGACCTGCTCCAGTACAGCCAGAAGGACCTGGATGCAGTGGTAAAGGCGACACAGGAGG
AGAACCGGGAGCTGAGGAGCAGGTGTGAGGAGCTCCACGGGAAGAACCTGGAACTGGGGAAGATCATGGA
CAGGTTCGAAGAGGTTGTGTACCAGGCCATGGAGGAAGTTCAGAAGCAGAAGGAACTTTCCAAAGCTGAA
ATCCAGAAAGTTCTAAAAGAAAAAGACCAACTTACCACAGATCTGAACTCCATGGAGAAGTCCTTCTCCG
ACCTCTTCAAGCGTTTTGAGAAACAGAAAGAGGTGATCGAGGGCTACCGCAAGAACGAAGAGTCACTGAA
GAAGTGCGTGGAGGATTACCTGGCAAGGATCACCCAGGAGGGCCAGAGGTACCAAGCCCTGAAGGCCCAC
GCGGAGGAGAAGCTGCAGCTGGCAAACGAGGAGATCGCCCAGGTCCGGAGCAAGGCCCAGGCGGAAGCGT
TGGCCCTCCAGGCCAGCCTGAGGAAGGAGCAGATGCGCATCCAGTCGCTGGAGAAGACAGTGGAGCAGAA
GACTAAAGAGAACGAGGAGCTGACCAGGATCTGCGACGACCTCATCTCCAAGATGGAGAAGATCTGA

SEQ ID NO: 94 is the nucleotide sequence of FGFR3-TACC3.

| | |
|---|---|
| 1 | gtcgcgggca gctggcgccg cgcggtcctg ctctgccggt cgcacggacg caccggcggg |
| 61 | ccgccggccg gagggacggg gcgggagctg ggcccgcgga cagcgagccg gagcgggagc |
| 121 | cgcgcgtagc gagccgggct ccggcgctcg ccagtctccc gagcggcgcc cgcctcccgc |
| 181 | cggtgcccgc gccgggccgt ggggggcagc atgcccgcgc gcgctgcctg aggacgccgc |
| 241 | ggcccccgcc cccgccatgg gcgcccctgc ctgcgccctc gcgctctgcg tggccgtggc |
| 301 | catcgtggcc ggcgcctcct cggagtcctt ggggacggag cagcgcgtcg tggggcgagc |
| 361 | ggcagaagtc ccgggcccag agcccggcca gcaggagcag ttggtcttcg gcagcgggga |
| 421 | tgctgtggag ctgagctgtc ccccgcccgg gggtggtccc atggggccca ctgtctgggt |
| 481 | caaggatggc acagggctgg tgccctcgga gcgtgtcctg gtggggcccc agcggctgca |
| 541 | ggtgctgaat gcctcccacg aggactccgg ggcctacagc tgccggcagc ggctcacgca |
| 601 | gcgcgtactg tgccacttca gtgtgcgggt gacagacgct ccatcctcgg gagatgacga |
| 661 | agacggggag gacgaggctg aggacacagg tgtggacaca ggggcccctt actggacacg |
| 721 | gcccgagcgg atggacaaga agctgctggc cgtgccggcc gccaacaccg tccgcttccg |
| 781 | ctgcccagcc gctggcaacc ccactccctc catctcctgg ctgaagaacg gcagggagtt |
| 841 | ccgcggcgag caccgcattg gaggcatcaa gctgcggcat cagcagtgga gcctggtcat |
| 901 | ggaaagcgtg gtgccctcgg accgcggcaa ctacacctgc gtcgtggaga acaagtttgg |
| 961 | cagcatccgg cagacgtaca cgctggacgt gctggagcgc tccccgcacc ggcccatcct |
| 1021 | gcaggcgggg ctgccggcca accagacggc ggtgctgggc agcgacgtgg agttccactg |
| 1081 | caaggtgtac agtgacgcac agccccacat ccagtggctc aagcacgtgg aggtgaatgg |
| 1141 | cagcaaggtg ggcccggacg gcacacccta cgttaccgtg ctcaagacgg cgggcgctaa |
| 1201 | caccaccgac aaggagctag aggttctctc cttgcacaac gtcacctttg aggacgccgg |
| 1261 | ggagtacacc tgcctggcgg gcaattctat tgggttttct catcactctg cgtggctggt |
| 1321 | ggtgctgcca gccgaggagg agctggtgga ggctgacgag gcgggcagtg tgtatgcagg |
| 1381 | catcctcagc tacggggtgg gcttcttcct gttcatcctg gtggtggcgg ctgtgacgct |
| 1441 | ctgccgcctg cgcagccccc ccaagaaagg cctgggctcc cccaccgtgc acaagatctc |
| 1501 | ccgcttcccg ctcaagcgac aggtgtccct ggagtccaac gcgtccatga gctccaacac |
| 1561 | accactggtg cgcatcgcaa ggctgtcctc aggggagggc cccacgctgg ccaatgtctc |
| 1621 | cgagctcgag ctgcctgccg accccaaatg ggagctgtct cgggcccggc tgaccctggg |
| 1681 | caagcccctt ggggagggct gcttcggcca ggtggtcatg gcggaggcca tcggcattga |
| 1741 | caaggaccgg gccgccaagc ctgtcaccgt agccgtgaag atgctgaaag acgatgccac |
| 1801 | tgacaaggac ctgtcggacc tggtgtctga gatggagatg atgaagatga tcgggaaaca |
| 1861 | caaaaacatc atcaacctgc tgggcgcctg cacgcagggc gggcccctgt acgtgctggt |
| 1921 | ggagtacgcg gccaagggta acctgcggga gtttctgcgg gcgcggcggc ccccgggcct |
| 1981 | ggactactcc ttcgacacct gcaagccgcc cgaggagcag ctcaccttca aggacctggt |
| 2041 | gtcctgtgcc taccaggtgg cccggggcat ggagtacttg gcctcccaga agtgcatcca |
| 2101 | cagggacctg gctgcccgca atgtgctggt gaccgaggac aacgtgatga agatcgcaga |
| 2161 | cttcgggctg gcccgggacg tgcacaacct cgactactac aagaagacaa ccaacggccg |
| 2221 | gctgcccgtg aagtggatgg cgcctgaggc cttgtttgac cgagtctaca ctcaccagag |
| 2281 | tgacgtctgg tcctttgggg tcctgctctg ggagatcttc acgctggggg gctccccgta |
| 2341 | ccccggcatc cctgtggagg agctcttcaa gctgctgaag gagggccacc gcatggacaa |
| 2401 | gcccgccaac tgcacacacg acctgtacat gatcatgcgg gagtgctggc atgccgcgcc |
| 2461 | ctcccagagg cccaccttca agcagctggt ggaggacctg gaccgtgtcc ttaccgtgac |
| 2521 | gtccaccgac tttaaggagt cggccttgag gaagcagtcc ttatacctca agttcgaccc |
| 2581 | cctcctgagg gacagtcctg gtagaccagt gcccgtggcc accgagacca gcagcatgca |
| 2641 | cggtgcaaat gagactccct caggacgtcc gcgggaagcc aagcttgtgg agttcgattt |
| 2701 | cttgggagca ctggacattc ctgtgccagg cccaccccca ggtgttcccg cgcctggggg |
| 2761 | cccacccctg tccaccggac ctatagtgga cctgctccag tacagccaga aggacctgga |
| 2821 | tgcagtggta aaggcgacac aggaggagaa ccgggagctg aggagcaggt gtgaggagct |
| 2881 | ccacgggaag aacctggaac tggggaagat catggacagg ttcgaagagg ttgtgtacca |
| 2941 | ggccatggag gaagttcaga agcagaagga actttccaaa gctgaaatcc agaaagttct |
| 3001 | aaaagaaaaa gaccaactta ccacagatct gaactccatg gagaagtcct tctccgacct |
| 3061 | cttcaagcgt tttgagaaac agaaagaggt gatcgagggc taccgcaaga acgaagagtc |
| 3121 | actgaagaag tgcgtggagg attacctggc aaggatcacc caggagggcc agaggtacca |
| 3181 | agccctgaag gcccacgcgg aggagaagct gcagctggca aacgaggaga tcgcccaggt |
| 3241 | ccggagcaag gcccaggcgg aagcgttggc cctccaggcc agcctgagga aggagcagat |
| 3301 | gcgcatccag tcgctggaga agacagtgga gcagaagact aaagagaacg aggagctgac |
| 3361 | caggatctgc gacgacctca tctccaagat ggagaagatc tgacctccac ggagccgctg |
| 3421 | tccccgcccc cctgctcccg tctgtctgtc ctgtctgatt ctcttaggtg tcatgttctt |
| 3481 | ttttctgtct tgtcttcaac ttttttaaaa actagattgc tttgaaaaca tgactcaata |
| 3541 | aaagtttcct ttcaatttaa acactgaaaa aaaaaaaa |

SEQ ID NO: 530 is the nucleotide sequence (cDNA) of FGFR3ex17-TACC3ex11. The sequence corresponding to FGFR3 is underlined. The sequence corresponding to TACC3 is italics:

ATGGGCGCCCCTGCCTGCGCCCTCGCGCTCTGCGTGGCCGTGGCCATCGT
GGCCGGCGCCTCCTCGGAGTCCTTGGGGACGGAGCAGCGCGTCGTGGGGC
GAGCGGCAGAAGTCCCGGGCCCAGAGCCCGGCCAGCAGGAGCAGTTGGTC
TTCGGCAGCGGGGATGCTGTGGAGCTGAGCTGTCCCCCGCCCGGGGGTGG
TCCCATGGGGCCCACTGTCTGGGTCAAGGATGGCACAGGGCTGGTGCCCT
CGGAGCGTGTCCTGGTGGGGCCCCAGCGGCTGCAGGTGCTGAATGCCTCC
CACGAGGACTCCGGGGCCTACAGCTGCCGGCAGCGGCTCACGCAGCGCGT
ACTGTGCCACTTCAGTGTGCGGGTGACAGACGCTCCATCCTCGGGAGATG
ACGAAGACGGGGAGGACGAGGCTGAGGACACAGGTGTGGACACAGGGGCC
CCTTACTGGACACGGCCCGAGCGGATGGACAAGAAGCTGCTGGCCGTGCC
GGCCGCCAACACCGTCCGCTTCCGCTGCCCAGCCGCTGGCAACCCCACTC
CCTCCATCTCCTGGCTGAAGAACGGCAGGGAGTTCCGCGGCGAGCACCGC
ATTGGAGGCATCAAGCTGCGGCATCAGCAGTGGAGCCTGGTCATGGAAAG
CGTGGTGCCCTCGGACCGCGGCAACTACACCTGCGTCGTGGAGAACAAGT
TTGGCAGCATCCGGCAGACGTACACGCTGGACGTGCTGGAGCGCTCCCCG
CACCGGCCCATCCTGCAGGCGGGGCTGCCGGCCAACCAGACGGCGGTGCT
GGGCAGCGACGTGGAGTTCCACTGCAAGGTGTACAGTGACGCACAGCCCC
ACATCCAGTGGCTCAAGCACGTGGAGGTGAATGGCAGCAAGGTGGGCCCG
GACGGCACACCCTACGTTACCGTGCTCAAGACGGCGGGCGCTAACACCAC
CGACAAGGAGCTAGAGGTTCTCTCCTTGCACAACGTCACCTTTGAGGACG
CCGGGGAGTACACCTGCCTGGCGGGCAATTCTATTGGGTTTTCTCATCAC
TCTGCGTGGCTGGTGGTGCTGCCAGCCGAGGAGGAGCTGGTGGAGGCTGA
CGAGGCGGGCAGTGTGTATGCAGGCATCCTCAGCTACGGGGTGGGCTTCT
TCCTGTTCATCCTGGTGGTGGCGGCTGTGACGCTCTGCCGCCTGCGCAGC
CCCCCCAAGAAAGGCCTGGGCTCCCCCACCGTGCACAAGATCTCCCGCTT
CCCGCTCAAGCGACAGGTGTCCCTGGAGTCCAACGCGTCCATGAGCTCCA
ACACACCACTGGTGCGCATCGCAAGGCTGTCCTCAGGGGAGGGCCCCACG
CTGGCCAATGTCTCCGAGCTCGAGCTGCCTGCCGACCCCAAATGGGAGCT
GTCTCGGGCCCGGCTGACCCTGGGCAAGCCCCTTGGGGAGGGCTGCTTCG
GCCAGGTGGTCATGGCGGAGGCCATCGGCATTGACAAGGACCGGGCCGCC
AAGCCTGTCACCGTAGCCGTGAAGATGCTGAAAGACGATGCCACTGACAA
GGACCTGTCGGACCTGGTGTCTGAGATGGAGATGATGAAGATGATCGGGA
AACACAAAAACATCATCAACCTGCTGGGCGCCTGCACGCAGGGCGGGCCC
CTGTACGTGCTGGTGGAGTACGCGGCCAAGGGTAACCTGCGGGAGTTTCT
GCGGGCGCGGCGGCCCCCGGGCCTGGACTACTCCTTCGACACCTGCAAGC
CGCCCGAGGAGCAGCTCACCTTCAAGGACCTGGTGTCCTGTGCCTACCAG
GTGGCCCGGGGCATGGAGTACTTGGCCTCCCAGAAGTGCATCCACAGGGA
CCTGGCTGCCCGCAATGTGCTGGTGACCGAGGACAACGTGATGAAGATCG
CAGACTTCGGGCTGGCCCGGGACGTGCACAACCTCGACTACTACAAGAAG
ACGACCAACGGCCGGCTGCCCGTGAAGTGGATGGCGCCTGAGGCCTTGTT
TGACCGAGTCTACACTCACCAGAGTGACGTCTGGTCCTTTGGGGTCCTGC
TCTGGGAGATCTTCACGCTGGGGGGCTCCCCGTACCCCGGCATCCCTGTG
GAGGAGCTCTTCAAGCTGCTGAAGGAGGGCCACCGCATGGACAAGCCCGC
CAACTGCACACACGACCTGTACATGATCATGCGGGAGTGCTGGCATGCCG
CGCCCTCCCAGAGGCCCACCTTCAAGCAGCTGGTGGAGGACCTGGACCGT
GTCCTTACCGTGACGTCCACCGACGTAAAGGCGACACAGGAGGAGAACCG
GGAGCTGAGGAGCAGGTGTGAGGAGCTCCACGGGAAGAACCTGGAACTGG
GGAAGATCATGGACAGGTTCGAAGAGGTTGTGTACCAGGCCATGGAGGAA
GTTCAGAAGCAGAAGGAACTTTCCAAAGCTGAAATCCAGAAAGTTCTAAA
AGAAAAAGACCAACTTACCACAGATCTGAACTCCATGGAGAAGTCCTTCT
CCGACCTCTTCAAGCGTTTTGAGAAACAGAAAGAGGTGATCGAGGGCTAC
CGCAAGAACGAAGAGTCACTGAAGAAGTGCGTGGAGGATTACCTGGCAAG
GATCACCCAGGAGGGCCAGAGGTACCAAGCCCTGAAGGCCCACGCGGAGG
AGAAGCTGCAGCTGGCAAACGAGGAGATCGCCCAGGTCCGGAGCAAGGCC
CAGGCGGAAGCGTTGGCCCTCCAGGCCAGCCTGAGGAAGGAGCAGATGCG
CATCCAGTCGCTGGAGAAGACAGTGGAGCAGAAGACTAAAGAGAACGAGG
AGCTGACCAGGATCTGCGACGACCTCATCTCCAAGATGGAGAAGATCTGA

SEQ ID NO: 531 is the nucleotide sequence (cDNA) of FGFR3ex17-TACC3ex8. The sequence corresponding to FGFR3 is underlined. The sequence corresponding to TACC3 is italicized:

ATGGGCGCCCCTGCCTGCGCCCTCGCGCTCTGCGTGGCCGTGGCCATCGT
GGCCGGCGCCTCCTCGGAGTCCTTGGGGACGGAGCAGCGCGTCGTGGGGC
GAGCGGCAGAAGTCCCGGGCCCAGAGCCCGGCCAGCAGGAGCAGTTGGTC
TTCGGCAGCGGGGATGCTGTGGAGCTGAGCTGTCCCCCGCCCGGGGGTGG
TCCCATGGGGCCCACTGTCTGGGTCAAGGATGGCACAGGGCTGGTGCCCT
CGGAGCGTGTCCTGGTGGGGCCCCAGCGGCTGCAGGTGCTGAATGCCTCC
CACGAGGACTCCGGGGCCTACAGCTGCCGGCAGCGGCTCACGCAGCGCGT
ACTGTGCCACTTCAGTGTGCGGGTGACAGACGCTCCATCCTCGGGAGATG
ACGAAGACGGGGAGGACGAGGCTGAGGACACAGGTGTGGACACAGGGGCC
CCTTACTGGACACGGCCCGAGCGGATGGACAAGAAGCTGCTGGCCGTGCC
GGCCGCCAACACCGTCCGCTTCCGCTGCCCAGCCGCTGGCAACCCCACTC
CCTCCATCTCCTGGCTGAAGAACGGCAGGGAGTTCCGCGGCGAGCACCGC
ATTGGAGGCATCAAGCTGCGGCATCAGCAGTGGAGCCTGGTCATGGAAAG
CGTGGTGCCCTCGGACCGCGGCAACTACACCTGCGTCGTGGAGAACAAGT
TTGGCAGCATCCGGCAGACGTACACGCTGGACGTGCTGGAGCGCTCCCCG
CACCGGCCCATCCTGCAGGCGGGGCTGCCGGCCAACCAGACGGCGGTGCT
GGGCAGCGACGTGGAGTTCCACTGCAAGGTGTACAGTGACGCACAGCCCC
ACATCCAGTGGCTCAAGCACGTGGAGGTGAATGGCAGCAAGGTGGGCCCG
GACGGCACACCCTACGTTACCGTGCTCAAGACGGCGGGCGCTAACACCAC
CGACAAGGAGCTAGAGGTTCTCTCCTTGCACAACGTCACCTTTGAGGACG
CCGGGGAGTACACCTGCCTGGCGGGCAATTCTATTGGGTTTTCTCATCAC
TCTGCGTGGCTGGTGGTGCTGCCAGCCGAGGAGGAGCTGGTGGAGGCTGA
CGAGGCGGGCAGTGTGTATGCAGGCATCCTCAGCTACGGGGTGGGCTTCT
TCCTGTTCATCCTGGTGGTGGCGGCTGTGACGCTCTGCCGCCTGCGCAGC
CCCCCCAAGAAAGGCCTGGGCTCCCCCACCGTGCACAAGATCTCCCGCTT
CCCGCTCAAGCGACAGGTGTCCCTGGAGTCCAACGCGTCCATGAGCTCCA
ACACACCACTGGTGCGCATCGCAAGGCTGTCCTCAGGGGAGGGCCCCACG
CTGGCCAATGTCTCCGAGCTCGAGCTGCCTGCCGACCCCAAATGGGAGCT
GTCTCGGGCCCGGCTGACCCTGGGCAAGCCCCTTGGGGAGGGCTGCTTCG
GCCAGGTGGTCATGGCGGAGGCCATCGGCATTGACAAGGACCGGGCCGCC
AAGCCTGTCACCGTAGCCGTGAAGATGCTGAAAGACGATGCCACTGACAA
GGACCTGTCGGACCTGGTGTCTGAGATGGAGATGATGAAGATGATCGGGA
AACACAAAAACATCATCAACCTGCTGGGCGCCTGCACGCAGGGCGGGCCC
CTGTACGTGCTGGTGGAGTACGCGGCCAAGGGTAACCTGCGGGAGTTTCT
GCGGGCGCGGCGGCCCCCGGGCCTGGACTACTCCTTCGACACCTGCAAGC
CGCCCGAGGAGCAGCTCACCTTCAAGGACCTGGTGTCCTGTGCCTACCAG
GTGGCCCGGGGCATGGAGTACTTGGCCTCCCAGAAGTGCATCCACAGGGA
CCTGGCTGCCCGCAATGTGCTGGTGACCGAGGACAACGTGATGAAGATCG
CAGACTTCGGGCTGGCCCGGGACGTGCACAACCTCGACTACTACAAGAAG
ACGACCAACGGCCGGCTGCCCGTGAAGTGGATGGCGCCTGAGGCCTTGTT
TGACCGAGTCTACACTCACCAGAGTGACGTCTGGTCCTTTGGGGTCCTGC
TCTGGGAGATCTTCACGCTGGGGGGCTCCCCGTACCCCGGCATCCCTGTG
GAGGAGCTCTTCAAGCTGCTGAAGGAGGGCCACCGCATGGACAAGCCCGC
CAACTGCACACACGACCTGTACATGATCATGCGGGAGTGCTGGCATGCCG
CGCCCTCCCAGAGGCCCACCTTCAAGCAGCTGGTGGAGGACCTGGACCGT
GTCCTTACCGTGACGTCCACCGACTTTAAGGAGTCGGCCTTGAGGAAGCA
GTCCTTATACCTCAAGTTCGACCCCCTCCTGAGGGACAGTCCTGGTAGAC
CAGTGCCCGTGGCCACCGAGACCAGCAGCATGCACGGTGCAAATGAGACT
CCCTCAGGACGTCCGCGGGAAGCCAAGCTTGTGGAGTTCGATTTCTTGGG
AGCACTGGACATTCCTGTGCCAGGCCCACCCCCAGGTGTTCCCGCGCCTG
GGGGCCCACCCCTGTCCACCGGACCTATAGTGGACCTGCTCCAGTACAGC
CAGAAGGACCTGGATGCAGTGGTAAAGGCGACACAGGAGGAGAACCGGGA
GCTGAGGAGCAGGTGTGAGGAGCTCCACGGGAAGAACCTGGAACTGGGGA
AGATCATGGACAGGTTCGAAGAGGTTGTGTACCAGGCCATGGAGGAAGTT
CAGAAGCAGAAGGAACTTTCCAAAGCTGAAATCCAGAAAGTTCTAAAAGA
AAAAGACCAACTTACCACAGATCTGAACTCCATGGAGAAGTCCTTCTCCG
ACCTCTTCAAGCGTTTTGAGAAACAGAAAGAGGTGATCGAGGGCTACCGC
AAGAACGAAGAGTCACTGAAGAAGTGCGTGGAGGATTACCTGGCAAGGAT
CACCCAGGAGGGCCAGAGGTACCAAGCCCTGAAGGCCCACGCGGAGGAGA
AGCTGCAGCTGGCAAACGAGGAGATCGCCCAGGTCCGGAGCAAGGCCCAG
GCGGAAGCGTTGGCCCTCCAGGCCAGCCTGAGGAAGGAGCAGATGCGCAT
CCAGTCGCTGGAGAAGACAGTGGAGCAGAAGACTAAAGAGAACGAGGAGC
TGACCAGGATCTGCGACGACCTCATCTCCAAGATGGAGAAGATCTGA

SEQ ID NO: 532 is the nucleotide sequence (cDNA) of FGFR3ex17-TACC3ex10. The sequence corresponding to FGFR3 is underlined. The sequence corresponding to TACC3 is italicized:

ATGGGCGCCCCTGCCTGCGCCCTCGCGCTCTGCGTGGCCGTGGCCATCGT
GGCCGGCGCCTCCTCGGAGTCCTTGGGGACGGAGCAGCGCGTCGTGGGGC
GAGCGGCAGAAGTCCCGGGCCCAGAGCCCGGCCAGCAGGAGCAGTTGGTC
TTCGGCAGCGGGGATGCTGTGGAGCTGAGCTGTCCCCCGCCCGGGGGTGG
TCCCATGGGGCCCACTGTCTGGGTCAAGGATGGCACAGGGCTGGTGCCCT
CGGAGCGTGTCCTGGTGGGGCCCCAGCGGCTGCAGGTGCTGAATGCCTCC
CACGAGGACTCCGGGGCCTACAGCTGCCGGCAGCGGCTCACGCAGCGCGT
ACTGTGCCACTTCAGTGTGCGGGTGACAGACGCTCCATCCTCGGGAGATG
ACGAAGACGGGGAGGACGAGGCTGAGGACACAGGTGTGGACACAGGGGCC
CCTTACTGGACACGGCCCGAGCGGATGGACAAGAAGCTGCTGGCCGTGCC
GGCCGCCAACACCGTCCGCTTCCGCTGCCCAGCCGCTGGCAACCCCACTC
CCTCCATCTCCTGGCTGAAGAACGGCAGGGAGTTCCGCGGCGAGCACCGC
ATTGGAGGCATCAAGCTGCGGCATCAGCAGTGGAGCCTGGTCATGGAAAG
CGTGGTGCCCTCGGACCGCGGCAACTACACCTGCGTCGTGGAGAACAAGT
TTGGCAGCATCCGGCAGACGTACACGCTGGACGTGCTGGAGCGCTCCCCG
CACCGGCCCATCCTGCAGGCGGGGCTGCCGGCCAACCAGACGGCGGTGCT
GGGCAGCGACGTGGAGTTCCACTGCAAGGTGTACAGTGACGCACAGCCCC
ACATCCAGTGGCTCAAGCACGTGGAGGTGAATGGCAGCAAGGTGGGCCCG
GACGGCACACCCTACGTTACCGTGCTCAAGACGGCGGGCGCTAACACCAC
CGACAAGGAGCTAGAGGTTCTCTCCTTGCACAACGTCACCTTTGAGGACG
CCGGGGAGTACACCTGCCTGGCGGGCAATTCTATTGGGTTTTCTCATCAC
TCTGCGTGGCTGGTGGTGCTGCCAGCCGAGGAGGAGCTGGTGGAGGCTGA
CGAGGCGGGCAGTGTGTATGCAGGCATCCTCAGCTACGGGGTGGGCTTCT
TCCTGTTCATCCTGGTGGTGGCGGCTGTGACGCTCTGCCGCCTGCGCAGC
CCCCCCAAGAAAGGCCTGGGCTCCCCCACCGTGCACAAGATCTCCCGCTT
CCCGCTCAAGCGACAGGTGTCCCTGGAGTCCAACGCGTCCATGAGCTCCA
ACACACCACTGGTGCGCATCGCAAGGCTGTCCTCAGGGGAGGGCCCCACG
CTGGCCAATGTCTCCGAGCTCGAGCTGCCTGCCGACCCCAAATGGGAGCT
GTCTCGGGCCCGGCTGACCCTGGGCAAGCCCCTTGGGGAGGGCTGCTTCG
GCCAGGTGGTCATGGCGGAGGCCATCGGCATTGACAAGGACCGGGCCGCC
AAGCCTGTCACCGTAGCCGTGAAGATGCTGAAAGACGATGCCACTGACAA
GGACCTGTCGGACCTGGTGTCTGAGATGGAGATGATGAAGATGATCGGGA
AACACAAAAACATCATCAACCTGCTGGGCGCCTGCACGCAGGGCGGGCCC
CTGTACGTGCTGGTGGAGTACGCGGCCAAGGGTAACCTGCGGGAGTTTCT
GCGGGCGCGGCGGCCCCCGGGCCTGGACTACTCCTTCGACACCTGCAAGC
CGCCCGAGGAGCAGCTCACCTTCAAGGACCTGGTGTCCTGTGCCTACCAG
GTGGCCCGGGGCATGGAGTACTTGGCCTCCCAGAAGTGCATCCACAGGGA
CCTGGCTGCCCGCAATGTGCTGGTGACCGAGGACAACGTGATGAAGATCG
CAGACTTCGGGCTGGCCCGGGACGTGCACAACCTCGACTACTACAAGAAG
ACGACCAACGGCCGGCTGCCCGTGAAGTGGATGGCGCCTGAGGCCTTGTT
TGACCGAGTCTACACTCACCAGAGTGACGTCTGGTCCTTTGGGGTCCTGC
TCTGGGAGATCTTCACGCTGGGGGGCTCCCCGTACCCCGGCATCCCTGTG
GAGGAGCTCTTCAAGCTGCTGAAGGAGGGCCACCGCATGGACAAGCCCGC
CAACTGCACACACGACCTGTACATGATCATGCGGGAGTGCTGGCATGCCG
CGCCCTCCCAGAGGCCCACCTTCAAGCAGCTGGTGGAGGACCTGGACCGT
GTCCTTACCGTGACGTCCACCGACGTGCCAGGCCCACCCCCAGGTGTTCC
CGCGCCTGGGGGCCCACCCCTGTCCACCGGACCTATAGTGGACCTGCTCC
AGTACAGCCAGAAGGACCTGGATGCAGTGGTAAAGGCGACACAGGAGGAG
AACCGGGAGCTGAGGAGCAGGTGTGAGGAGCTCCACGGGAAGAACCTGGA
ACTGGGGAAGATCATGGACAGGTTCGAAGAGGTTGTGTACCAGGCCATGG
AGGAAGTTCAGAAGCAGAAGGAACTTTCCAAAGCTGAAATCCAGAAAGTT
CTAAAAGAAAAAGACCAACTTACCACAGATCTGAACTCCATGGAGAAGTC
CTTCTCCGACCTCTTCAAGCGTTTTGAGAAACAGAAAGAGGTGATCGAGG
GCTACCGCAAGAACGAAGAGTCACTGAAGAAGTGCGTGGAGGATTACCTG
GCAAGGATCACCCAGGAGGGCCAGAGGTACCAAGCCCTGAAGGCCCACGC
GGAGGAGAAGCTGCAGCTGGCAAACGAGGAGATCGCCCAGGTCCGGAGCA
AGGCCCAGGCGGAAGCGTTGGCCCTCCAGGCCAGCCTGAGGAAGGAGCAG
ATGCGCATCCAGTCGCTGGAGAAGACAGTGGAGCAGAAGACTAAAGAGAA
CGAGGAGCTGACCAGGATCTGCGACGACCTCATCTCCAAGATGGAGAAGA
TCTGA

SEQ ID NO: 533 is the nucleotide sequence (cDNA) of FGFR3ex17-TACC3ex6. The sequence corresponding to FGFR3 is underlined. The sequence corresponding to TACC3 is italicized:

ATGGGCGCCCCTGCCTGCGCCCTCGCGCTCTGCGTGGCCGTGGCCATCGT
GGCCGGCGCCTCCTCGGAGTCCTTGGGGACGGAGCAGCGCGTCGTGGGGC
GAGCGGCAGAAGTCCCGGGCCCAGAGCCCGGCCAGCAGGAGCAGTTGGTC
TTCGGCAGCGGGGATGCTGTGGAGCTGAGCTGTCCCCCGCCCGGGGGTGG
TCCCATGGGGCCCACTGTCTGGGTCAAGGATGGCACAGGGCTGGTGCCCT
CGGAGCGTGTCCTGGTGGGGCCCCAGCGGCTGCAGGTGCTGAATGCCTCC
CACGAGGACTCCGGGGCCTACAGCTGCCGGCAGCGGCTCACGCAGCGCGT
ACTGTGCCACTTCAGTGTGCGGGTGACAGACGCTCCATCCTCGGGAGATG
ACGAAGACGGGGAGGACGAGGCTGAGGACACAGGTGTGGACACAGGGGCC
CCTTACTGGACACGGCCCGAGCGGATGGACAAGAAGCTGCTGGCCGTGCC
GGCCGCCAACACCGTCCGCTTCCGCTGCCCAGCCGCTGGCAACCCCACTC
CCTCCATCTCCTGGCTGAAGAACGGCAGGGAGTTCCGCGGCGAGCACCGC
ATTGGAGGCATCAAGCTGCGGCATCAGCAGTGGAGCCTGGTCATGGAAAG
CGTGGTGCCCTCGGACCGCGGCAACTACACCTGCGTCGTGGAGAACAAGT
TTGGCAGCATCCGGCAGACGTACACGCTGGACGTGCTGGAGCGCTCCCCG
CACCGGCCCATCCTGCAGGCGGGGCTGCCGGCCAACCAGACGGCGGTGCT
GGGCAGCGACGTGGAGTTCCACTGCAAGGTGTACAGTGACGCACAGCCCC
ACATCCAGTGGCTCAAGCACGTGGAGGTGAATGGCAGCAAGGTGGGCCCG
GACGGCACACCCTACGTTACCGTGCTCAAGACGGCGGGCGCTAACACCAC
CGACAAGGAGCTAGAGGTTCTCTCCTTGCACAACGTCACCTTTGAGGACG
CCGGGGAGTACACCTGCCTGGCGGGCAATTCTATTGGGTTTTCTCATCAC
TCTGCGTGGCTGGTGGTGCTGCCAGCCGAGGAGGAGCTGGTGGAGGCTGA
CGAGGCGGGCAGTGTGTATGCAGGCATCCTCAGCTACGGGGTGGGCTTCT
TCCTGTTCATCCTGGTGGTGGCGGCTGTGACGCTCTGCCGCCTGCGCAGC
CCCCCCAAGAAAGGCCTGGGCTCCCCCACCGTGCACAAGATCTCCCGCTT
CCCGCTCAAGCGACAGGTGTCCCTGGAGTCCAACGCGTCCATGAGCTCCA
ACACACCACTGGTGCGCATCGCAAGGCTGTCCTCAGGGGAGGGCCCCACG
CTGGCCAATGTCTCCGAGCTCGAGCTGCCTGCCGACCCCAAATGGGAGCT
GTCTCGGGCCCGGCTGACCCTGGGCAAGCCCCTTGGGGAGGGCTGCTTCG
GCCAGGTGGTCATGGCGGAGGCCATCGGCATTGACAAGGACCGGGCCGCC
AAGCCTGTCACCGTAGCCGTGAAGATGCTGAAAGACGATGCCACTGACAA
GGACCTGTCGGACCTGGTGTCTGAGATGGAGATGATGAAGATGATCGGGA
AACACAAAAACATCATCAACCTGCTGGGCGCCTGCACGCAGGGCGGGCCC
CTGTACGTGCTGGTGGAGTACGCGGCCAAGGGTAACCTGCGGGAGTTTCT
GCGGGCGCGGCGGCCCCCGGGCCTGGACTACTCCTTCGACACCTGCAAGC
CGCCCGAGGAGCAGCTCACCTTCAAGGACCTGGTGTCCTGTGCCTACCAG
GTGGCCCGGGGCATGGAGTACTTGGCCTCCCAGAAGTGCATCCACAGGGA
CCTGGCTGCCCGCAATGTGCTGGTGACCGAGGACAACGTGATGAAGATCG
CAGACTTCGGGCTGGCCCGGGACGTGCACAACCTCGACTACTACAAGAAG
ACGACCAACGGCCGGCTGCCCGTGAAGTGGATGGCGCCTGAGGCCTTGTT
TGACCGAGTCTACACTCACCAGAGTGACGTCTGGTCCTTTGGGGTCCTGC
TCTGGGAGATCTTCACGCTGGGGGGCTCCCCGTACCCCGGCATCCCTGTG
GAGGAGCTCTTCAAGCTGCTGAAGGAGGGCCACCGCATGGACAAGCCCGC
CAACTGCACACACGACCTGTACATGATCATGCGGGAGTGCTGGCATGCCG
CGCCCTCCCAGAGGCCCACCTTCAAGCAGCTGGTGGAGGACCTGGACCGT
GTCCTTACCGTGACGTCCACCGACGAGAGAGCCTTGAACTCTGCCAGCAC
CTCGCTTCCCACAAGCTGTCCAGGCAGTGAGCCAGTGCCCACCCATCAGC
AGGGGCAGCCTGCCTTGGAGCTGAAAGAGGAGAGCTTCAGAGACCCCGCT
GAGGTTCTAGGCACGGGCGCGGAGGTGGATTACCTGGAGCAGTTTGGAAC
TTCCTCGTTTAAGGAGTCGGCCTTGAGGAAGCAGTCCTTATACCTCAAGT
TCGACCCCCTCCTGAGGGACAGTCCTGGTAGACCAGTGCCCGTGGCCACC
GAGACCAGCAGCATGCACGGTGCAAATGAGACTCCCTCAGGACGTCCGCG
GGAAGCCAAGCTTGTGGAGTTCGATTTCTTGGGAGCACTGGACATTCCTG
TGCCAGGCCCACCCCCAGGTGTTCCCGCGCCTGGGGGCCCACCCCTGTCC
ACCGGACCTATAGTGGACCTGCTCCAGTACAGCCAGAAGGACCTGGATGC
AGTGGTAAAGGCGACACAGGAGGAGAACCGGGAGCTGAGGAGCAGGTGTG
AGGAGCTCCACGGGAAGAACCTGGAACTGGGGAAGATCATGGACAGGTTC
GAAGAGGTTGTGTACCAGGCCATGGAGGAAGTTCAGAAGCAGAAGGAACT
TTCCAAAGCTGAAATCCAGAAAGTTCTAAAAGAAAAAGACCAACTTACCA
CAGATCTGAACTCCATGGAGAAGTCCTTCTCCGACCTCTTCAAGCGTTTT
GAGAAACAGAAAGAGGTGATCGAGGGCTACCGCAAGAACGAAGAGTCACT
GAAGAAGTGCGTGGAGGATTACCTGGCAAGGATCACCCAGGAGGGCCAGA
GGTACCAAGCCCTGAAGGCCCACGCGGAGGAGAAGCTGCAGCTGGCAAAC
GAGGAGATCGCCCAGGTCCGGAGCAAGGCCCAGGCGGAAGCGTTGGCCCT
CCAGGCCAGCCTGAGGAAGGAGCAGATGCGCATCCAGTCGCTGGAGAAGA
CAGTGGAGCAGAAGACTAAAGAGAACGAGGAGCTGACCAGGATCTGCGAC
GACCTCATCTCCAAGATGGAGAAGATCTGA

SEQ ID NO: 534 is the nucleotide sequence (cDNA) of FGFR3ex18-TACC3ex13. The sequence corresponding to FGFR3 is underlined. The sequence corresponding to TACC3 is italicized:

ATGGGCGCCCCTGCCTGCGCCCTCGCGCTCTGCGTGGCCGTGGCCATCGT
GGCCGGCGCCTCCTCGGAGTCCTTGGGGACGGAGCAGCGCGTCGTGGGGC
GAGCGGCAGAAGTCCCGGGCCCAGAGCCCGGCCAGCAGGAGCAGTTGGTC
TTCGGCAGCGGGGATGCTGTGGAGCTGAGCTGTCCCCCGCCCGGGGGTGG
TCCCATGGGGCCCACTGTCTGGGTCAAGGATGGCACAGGGCTGGTGCCCT
CGGAGCGTGTCCTGGTGGGGCCCCAGCGGCTGCAGGTGCTGAATGCCTCC
CACGAGGACTCCGGGGCCTACAGCTGCCGGCAGCGGCTCACGCAGCGCGT
ACTGTGCCACTTCAGTGTGCGGGTGACAGACGCTCCATCCTCGGGAGATG
ACGAAGACGGGGAGGACGAGGCTGAGGACACAGGTGTGGACACAGGGGCC
CCTTACTGGACACGGCCCGAGCGGATGGACAAGAAGCTGCTGGCCGTGCC
GGCCGCCAACACCGTCCGCTTCCGCTGCCCAGCCGCTGGCAACCCCACTC
CCTCCATCTCCTGGCTGAAGAACGGCAGGGAGTTCCGCGGCGAGCACCGC
ATTGGAGGCATCAAGCTGCGGCATCAGCAGTGGAGCCTGGTCATGGAAAG
CGTGGTGCCCTCGGACCGCGGCAACTACACCTGCGTCGTGGAGAACAAGT
TTGGCAGCATCCGGCAGACGTACACGCTGGACGTGCTGGAGCGCTCCCCG
CACCGGCCCATCCTGCAGGCGGGGCTGCCGGCCAACCAGACGGCGGTGCT
GGGCAGCGACGTGGAGTTCCACTGCAAGGTGTACAGTGACGCACAGCCCC
ACATCCAGTGGCTCAAGCACGTGGAGGTGAATGGCAGCAAGGTGGGCCCG
GACGGCACACCCTACGTTACCGTGCTCAAGACGGCGGGCGCTAACACCAC
CGACAAGGAGCTAGAGGTTCTCTCCTTGCACAACGTCACCTTTGAGGACG
CCGGGGAGTACACCTGCCTGGCGGGCAATTCTATTGGGTTTTCTCATCAC
TCTGCGTGGCTGGTGGTGCTGCCAGCCGAGGAGGAGCTGGTGGAGGCTGA
CGAGGCGGGCAGTGTGTATGCAGGCATCCTCAGCTACGGGGTGGGCTTCT
TCCTGTTCATCCTGGTGGTGGCGGCTGTGACGCTCTGCCGCCTGCGCAGC
CCCCCCAAGAAAGGCCTGGGCTCCCCCACCGTGCACAAGATCTCCCGCTT
CCCGCTCAAGCGACAGGTGTCCCTGGAGTCCAACGCGTCCATGAGCTCCA
ACACACCACTGGTGCGCATCGCAAGGCTGTCCTCAGGGGAGGGCCCCACG
CTGGCCAATGTCTCCGAGCTCGAGCTGCCTGCCGACCCCAAATGGGAGCT
GTCTCGGGCCCGGCTGACCCTGGGCAAGCCCCTTGGGGAGGGCTGCTTCG
GCCAGGTGGTCATGGCGGAGGCCATCGGCATTGACAAGGACCGGGCCGCC
AAGCCTGTCACCGTAGCCGTGAAGATGCTGAAAGACGATGCCACTGACAA
GGACCTGTCGGACCTGGTGTCTGAGATGGAGATGATGAAGATGATCGGGA
AACACAAAAACATCATCAACCTGCTGGGCGCCTGCACGCAGGGCGGGCCC
CTGTACGTGCTGGTGGAGTACGCGGCCAAGGGTAACCTGCGGGAGTTTCT
GCGGGCGCGGCGGCCCCCGGGCCTGGACTACTCCTTCGACACCTGCAAGC
CGCCCGAGGAGCAGCTCACCTTCAAGGACCTGGTGTCCTGTGCCTACCAG
GTGGCCCGGGGCATGGAGTACTTGGCCTCCCAGAAGTGCATCCACAGGGA
CCTGGCTGCCCGCAATGTGCTGGTGACCGAGGACAACGTGATGAAGATCG
CAGACTTCGGGCTGGCCCGGGACGTGCACAACCTCGACTACTACAAGAAG
ACGACCAACGGCCGGCTGCCCGTGAAGTGGATGGCGCCTGAGGCCTTGTT
TGACCGAGTCTACACTCACCAGAGTGACGTCTGGTCCTTTGGGGTCCTGC
TCTGGGAGATCTTCACGCTGGGGGGCTCCCCGTACCCCGGCATCCCTGTG
GAGGAGCTCTTCAAGCTGCTGAAGGAGGGCCACCGCATGGACAAGCCCGC
CAACTGCACACACGACCTGTACATGATCATGCGGGAGTGCTGGCATGCCG
CGCCCTCCCAGAGGCCCACCTTCAAGCAGCTGGTGGAGGACCTGGACCGT
GTCCTTACCGTGACGTCCACCGACGAGTACCTGGACCTGTCGGCGCCTTT
CGAGCAGTACTCCCCGGGTGGCCAGGACACCCCCAGAAGCAGAAGGAACT
TTCCAAAGCTGAAATCCAGAAAGTTCTAAAAGAAAAAGACCAACTTACCA
CAGATCTGAACTCCATGGAGAAGTCCTTCTCCGACCTCTTCAAGCGTTTT
GAGAAACAGAAAGAGGTGATCGAGGGCTACCGCAAGAACGAAGAGTCACT
GAAGAAGTGCGTGGAGGATTACCTGGCAAGGATCACCCAGGAGGGCCAGA
GGTACCAAGCCCTGAAGGCCCACGCGGAGGAGAAGCTGCAGCTGGCAAAC
GAGGAGATCGCCCAGGTCCGGAGCAAGGCCCAGGCGGAAGCGTTGGCCCT
CCAGGCCAGCCTGAGGAAGGAGCAGATGCGCATCCAGTCGCTGGAGAAGA
CAGTGGAGCAGAAGACTAAAGAGAACGAGGAGCTGACCAGGATCTGCGAC
GACCTCATCTCCAAGATGGAGAAGATCTGA

SEQ ID NO: 535 is the nucleotide sequence (cDNA) of FGFR3ex18-TACC3ex9_INS66BP. The sequence corresponding to FGFR3 is underlined. The sequence corresponding to TACC3 is italicized. The sequence corresponding the the 66 bp intronic insert is double underlined:

ATGGGCGCCCCTGCCTGCGCCCTCGCGCTCTGCGTGGCCGTGGCCATCGT
GGCCGGCGCCTCCTCGGAGTCCTTGGGGACGGAGCAGCGCGTCGTGGGGC
GAGCGGCAGAAGTCCCGGGCCCAGAGCCCGGCCAGCAGGAGCAGTTGGTC
TTCGGCAGCGGGGATGCTGTGGAGCTGAGCTGTCCCCCGCCCGGGGGTGG
TCCCATGGGGCCCACTGTCTGGGTCAAGGATGGCACAGGGCTGGTGCCCT
CGGAGCGTGTCCTGGTGGGGCCCCAGCGGCTGCAGGTGCTGAATGCCTCC
CACGAGGACTCCGGGGCCTACAGCTGCCGGCAGCGGCTCACGCAGCGCGT
ACTGTGCCACTTCAGTGTGCGGGTGACAGACGCTCCATCCTCGGGAGATG
ACGAAGACGGGGAGGACGAGGCTGAGGACACAGGTGTGGACACAGGGGCC
CCTTACTGGACACGGCCCGAGCGGATGGACAAGAAGCTGCTGGCCGTGCC
GGCCGCCAACACCGTCCGCTTCCGCTGCCCAGCCGCTGGCAACCCCACTC
CCTCCATCTCCTGGCTGAAGAACGGCAGGGAGTTCCGCGGCGAGCACCGC
ATTGGAGGCATCAAGCTGCGGCATCAGCAGTGGAGCCTGGTCATGGAAAG
CGTGGTGCCCTCGGACCGCGGCAACTACACCTGCGTCGTGGAGAACAAGT
TTGGCAGCATCCGGCAGACGTACACGCTGGACGTGCTGGAGCGCTCCCCG
CACCGGCCCATCCTGCAGGCGGGGCTGCCGGCCAACCAGACGGCGGTGCT
GGGCAGCGACGTGGAGTTCCACTGCAAGGTGTACAGTGACGCACAGCCCC
ACATCCAGTGGCTCAAGCACGTGGAGGTGAATGGCAGCAAGGTGGGCCCG
GACGGCACACCCTACGTTACCGTGCTCAAGACGGCGGGCGCTAACACCAC
CGACAAGGAGCTAGAGGTTCTCTCCTTGCACAACGTCACCTTTGAGGACG
CCGGGGAGTACACCTGCCTGGCGGGCAATTCTATTGGGTTTTCTCATCAC
TCTGCGTGGCTGGTGGTGCTGCCAGCCGAGGAGGAGCTGGTGGAGGCTGA
CGAGGCGGGCAGTGTGTATGCAGGCATCCTCAGCTACGGGGTGGGCTTCT
TCCTGTTCATCCTGGTGGTGGCGGCTGTGACGCTCTGCCGCCTGCGCAGC
CCCCCCAAGAAAGGCCTGGGCTCCCCCACCGTGCACAAGATCTCCCGCTT
CCCGCTCAAGCGACAGGTGTCCCTGGAGTCCAACGCGTCCATGAGCTCCA
ACACACCACTGGTGCGCATCGCAAGGCTGTCCTCAGGGGAGGGCCCCACG
CTGGCCAATGTCTCCGAGCTCGAGCTGCCTGCCGACCCCAAATGGGAGCT
GTCTCGGGCCCGGCTGACCCTGGGCAAGCCCCTTGGGGAGGGCTGCTTCG
GCCAGGTGGTCATGGCGGAGGCCATCGGCATTGACAAGGACCGGGCCGCC
AAGCCTGTCACCGTAGCCGTGAAGATGCTGAAAGACGATGCCACTGACAA
GGACCTGTCGGACCTGGTGTCTGAGATGGAGATGATGAAGATGATCGGGA
AACACAAAAACATCATCAACCTGCTGGGCGCCTGCACGCAGGGCGGGCCC
CTGTACGTGCTGGTGGAGTACGCGGCCAAGGGTAACCTGCGGGAGTTTCT
GCGGGCGCGGCGGCCCCCGGGCCTGGACTACTCCTTCGACACCTGCAAGC
CGCCCGAGGAGCAGCTCACCTTCAAGGACCTGGTGTCCTGTGCCTACCAG
GTGGCCCGGGGCATGGAGTACTTGGCCTCCCAGAAGTGCATCCACAGGGA
CCTGGCTGCCCGCAATGTGCTGGTGACCGAGGACAACGTGATGAAGATCG
CAGACTTCGGGCTGGCCCGGGACGTGCACAACCTCGACTACTACAAGAAG
ACGACCAACGGCCGGCTGCCCGTGAAGTGGATGGCGCCTGAGGCCTTGTT
TGACCGAGTCTACACTCACCAGAGTGACGTCTGGTCCTTTGGGGTCCTGC
TCTGGGAGATCTTCACGCTGGGGGGCTCCCCGTACCCCGGCATCCCTGTG
GAGGAGCTCTTCAAGCTGCTGAAGGAGGGCCACCGCATGGACAAGCCCGC
CAACTGCACACACGACCTGTACATGATCATGCGGGAGTGCTGGCATGCCG
CGCCCTCCCAGAGGCCCACCTTCAAGCAGCTGGTGGAGGACCTGGACCGT
GTCCTTACCGTGACGTCCACCGACGAGTACCTGGACCTGTCGGCGCAGGA
GCAACGGCAGCCTACCCTCCAGCCACAGGGCTGCTGCCTTGCTGGTTACA
GCCACCGTTTCTCTAGCATGCACGGTGCAAATGAGACTCCCTCAGGACGT
CCGCGGGAAGCCAAGCTTGTGGAGTTCGATTTCTTGGGAGCACTGGACAT
TCCTGTGCCAGGCCCACCCCCAGGTGTTCCCGCGCCTGGGGGCCCACCCC
TGTCCACCGGACCTATAGTGGACCTGCTCCAGTACAGCCAGAAGGACCTG
GATGCAGTGGTAAAGGCGACACAGGAGGAGAACCGGGAGCTGAGGAGCAG
GTGTGAGGAGCTCCACGGGAAGAACCTGGAACTGGGGAAGATCATGGACA
GGTTCGAAGAGGTTGTGTACCAGGCCATGGAGGAAGTTCAGAAGCAGAAG
GAACTTTCCAAAGCTGAAATCCAGAAAGTTCTAAAAGAAAAAGACCAACT
TACCACAGATCTGAACTCCATGGAGAAGTCCTTCTCCGACCTCTTCAAGC
GTTTTGAGAAACAGAAAGAGGTGATCGAGGGCTACCGCAAGAACGAAGAG
TCACTGAAGAAGTGCGTGGAGGATTACCTGGCAAGGATCACCCAGGAGGG
CCAGAGGTACCAAGCCCTGAAGGCCCACGCGGAGGAGAAGCTGCAGCTGG
CAAACGAGGAGATCGCCCAGGTCCGGAGCAAGGCCCAGGCGGAAGCGTTG
GCCCTCCAGGCCAGCCTGAGGAAGGAGCAGATGCGCATCCAGTCGCTGGA
GAAGACAGTGGAGCAGAAGACTAAAGAGAACGAGGAGCTGACCAGGATCT
GCGACGACCTCATCTCCAAGATGGAGAAGATCTGA

SEQ ID NO: 536 is the nucleotide sequence (cDNA) of FGFR3ex18-TACC3ex5. The sequence corresponding to FGFR3 is underlined. The sequence corresponding to TACC3 is italicized:

ATGGGCGCCCCTGCCTGCGCCCTCGCGCTCTGCGTGGCCGTGGCCATCGT
GGCCGGCGCCTCCTCGGAGTCCTTGGGGACGGAGCAGCGCGTCGTGGGGC
GAGCGGCAGAAGTCCCGGGCCCAGAGCCCGGCCAGCAGGAGCAGTTGGTC
TTCGGCAGCGGGGATGCTGTGGAGCTGAGCTGTCCCCCGCCCGGGGGTGG
TCCCATGGGGCCCACTGTCTGGGTCAAGGATGGCACAGGGCTGGTGCCCT
CGGAGCGTGTCCTGGTGGGGCCCCAGCGGCTGCAGGTGCTGAATGCCTCC
CACGAGGACTCCGGGGCCTACAGCTGCCGGCAGCGGCTCACGCAGCGCGT
ACTGTGCCACTTCAGTGTGCGGGTGACAGACGCTCCATCCTCGGGAGATG
ACGAAGACGGGGAGGACGAGGCTGAGGACACAGGTGTGGACACAGGGGCC
CCTTACTGGACACGGCCCGAGCGGATGGACAAGAAGCTGCTGGCCGTGCC
GGCCGCCAACACCGTCCGCTTCCGCTGCCCAGCCGCTGGCAACCCCACTC
CCTCCATCTCCTGGCTGAAGAACGGCAGGGAGTTCCGCGGCGAGCACCGC
ATTGGAGGCATCAAGCTGCGGCATCAGCAGTGGAGCCTGGTCATGGAAAG
CGTGGTGCCCTCGGACCGCGGCAACTACACCTGCGTCGTGGAGAACAAGT
TTGGCAGCATCCGGCAGACGTACACGCTGGACGTGCTGGAGCGCTCCCCG
CACCGGCCCATCCTGCAGGCGGGGCTGCCGGCCAACCAGACGGCGGTGCT
GGGCAGCGACGTGGAGTTCCACTGCAAGGTGTACAGTGACGCACAGCCCC
ACATCCAGTGGCTCAAGCACGTGGAGGTGAATGGCAGCAAGGTGGGCCCG
GACGGCACACCCTACGTTACCGTGCTCAAGACGGCGGGCGCTAACACCAC
CGACAAGGAGCTAGAGGTTCTCTCCTTGCACAACGTCACCTTTGAGGACG
CCGGGGAGTACACCTGCCTGGCGGGCAATTCTATTGGGTTTTCTCATCAC
TCTGCGTGGCTGGTGGTGCTGCCAGCCGAGGAGGAGCTGGTGGAGGCTGA
CGAGGCGGGCAGTGTGTATGCAGGCATCCTCAGCTACGGGGTGGGCTTCT
TCCTGTTCATCCTGGTGGTGGCGGCTGTGACGCTCTGCCGCCTGCGCAGC
CCCCCCAAGAAAGGCCTGGGCTCCCCCACCGTGCACAAGATCTCCCGCTT
CCCGCTCAAGCGACAGGTGTCCCTGGAGTCCAACGCGTCCATGAGCTCCA
ACACACCACTGGTGCGCATCGCAAGGCTGTCCTCAGGGGAGGGCCCCACG
CTGGCCAATGTCTCCGAGCTCGAGCTGCCTGCCGACCCCAAATGGGAGCT
GTCTCGGGCCCGGCTGACCCTGGGCAAGCCCCTTGGGGAGGGCTGCTTCG
GCCAGGTGGTCATGGCGGAGGCCATCGGCATTGACAAGGACCGGGCCGCC
AAGCCTGTCACCGTAGCCGTGAAGATGCTGAAAGACGATGCCACTGACAA
GGACCTGTCGGACCTGGTGTCTGAGATGGAGATGATGAAGATGATCGGGA
AACACAAAAACATCATCAACCTGCTGGGCGCCTGCACGCAGGGCGGGCCC
CTGTACGTGCTGGTGGAGTACGCGGCCAAGGGTAACCTGCGGGAGTTTCT
GCGGGCGCGGCGGCCCCCGGGCCTGGACTACTCCTTCGACACCTGCAAGC
CGCCCGAGGAGCAGCTCACCTTCAAGGACCTGGTGTCCTGTGCCTACCAG
GTGGCCCGGGGCATGGAGTACTTGGCCTCCCAGAAGTGCATCCACAGGGA
CCTGGCTGCCCGCAATGTGCTGGTGACCGAGGACAACGTGATGAAGATCG
CAGACTTCGGGCTGGCCCGGGACGTGCACAACCTCGACTACTACAAGAAG
ACGACCAACGGCCGGCTGCCCGTGAAGTGGATGGCGCCTGAGGCCTTGTT
TGACCGAGTCTACACTCACCAGAGTGACGTCTGGTCCTTTGGGGTCCTGC
TCTGGGAGATCTTCACGCTGGGGGGCTCCCCGTACCCCGGCATCCCTGTG
GAGGAGCTCTTCAAGCTGCTGAAGGAGGGCCACCGCATGGACAAGCCCGC
CAACTGCACACACGACCTGTACATGATCATGCGGGAGTGCTGGCATGCCG
CGCCCTCCCAGAGGCCCACCTTCAAGCAGCTGGTGGAGGACCTGGACCGT
GTCCTTACCGTGACGTCCACCGACGAGTACCTGGACCTGTCGGCGCCTGT
GGTGCAGTTGGCAGCCGAGACCCCAACAGCAGAGAGCAAGGAGAGAGCCT
TGAACTCTGCCAGCACCTCGCTTCCCACAAGCTGTCCAGGCAGTGAGCCA
GTGCCCACCCATCAGCAGGGGCAGCCTGCCTTGGAGCTGAAAGAGGAGAG
CTTCAGAGACCCCGCTGAGGTTCTAGGCACGGGCGCGGAGGTGGATTACC
TGGAGCAGTTTGGAACTTCCTCGTTTAAGGAGTCGGCCTTGAGGAAGCAG
TCCTTATACCTCAAGTTCGACCCCCTCCTGAGGGACAGTCCTGGTAGACC
AGTGCCCGTGGCCACCGAGACCAGCAGCATGCACGGTGCAAATGAGACTC
CCTCAGGACGTCCGCGGGAAGCCAAGCTTGTGGAGTTCGATTTCTTGGGA
GCACTGGACATTCCTGTGCCAGGCCCACCCCCAGGTGTTCCCGCGCCTGG
GGGCCCACCCCTGTCCACCGGACCTATAGTGGACCTGCTCCAGTACAGCC
AGAAGGACCTGGATGCAGTGGTAAAGGCGACACAGGAGGAGAACCGGGAG
CTGAGGAGCAGGTGTGAGGAGCTCCACGGGAAGAACCTGGAACTGGGGAA
GATCATGGACAGGTTCGAAGAGGTTGTGTACCAGGCCATGGAGGAAGTTC
AGAAGCAGAAGGAACTTTCCAAAGCTGAAATCCAGAAAGTTCTAAAAGAA
AAAGACCAACTTACCACAGATCTGAACTCCATGGAGAAGTCCTTCTCCGA
CCTCTTCAAGCGTTTTGAGAAACAGAAAGAGGTGATCGAGGGCTACCGCA
AGAACGAAGAGTCACTGAAGAAGTGCGTGGAGGATTACCTGGCAAGGATC
ACCCAGGAGGGCCAGAGGTACCAAGCCCTGAAGGCCCACGCGGAGGAGAA
GCTGCAGCTGGCAAACGAGGAGATCGCCCAGGTCCGGAGCAAGGCCCAGG
CGGAAGCGTTGGCCCTCCAGGCCAGCCTGAGGAAGGAGCAGATGCGCATC
CAGTCGCTGGAGAAGACAGTGGAGCAGAAGACTAAAGAGAACGAGGAGCT
GACCAGGATCTGCGACGACCTCATCTCCAAGATGGAGAAGATCTGA

SEQ ID NO: 537 is the nucleotide sequence (cDNA) of FGFR3ex18-TACC3ex5_INS33 bp. The sequence corresponding to FGFR3 is underlined. The sequence corresponding to TACC3 is italicized. The sequence corresponding the the 33 bp intronic insert is double underlined:

ATGGGCGCCCCTGCCTGCGCCCTCGCGCTCTGCGTGGCCGTGGCCATCGT
GGCCGGCGCCTCCTCGGAGTCCTTGGGGACGGAGCAGCGCGTCGTGGGGC
GAGCGGCAGAAGTCCCGGGCCCAGAGCCCGGCCAGCAGGAGCAGTTGGTC
TTCGGCAGCGGGGATGCTGTGGAGCTGAGCTGTCCCCCGCCCGGGGGTGG
TCCCATGGGGCCCACTGTCTGGGTCAAGGATGGCACAGGGCTGGTGCCCT
CGGAGCGTGTCCTGGTGGGGCCCCAGCGGCTGCAGGTGCTGAATGCCTCC
CACGAGGACTCCGGGGCCTACAGCTGCCGGCAGCGGCTCACGCAGCGCGT
ACTGTGCCACTTCAGTGTGCGGGTGACAGACGCTCCATCCTCGGGAGATG
ACGAAGACGGGGAGGACGAGGCTGAGGACACAGGTGTGGACACAGGGGCC
CCTTACTGGACACGGCCCGAGCGGATGGACAAGAAGCTGCTGGCCGTGCC
GGCCGCCAACACCGTCCGCTTCCGCTGCCCAGCCGCTGGCAACCCCACTC
CCTCCATCTCCTGGCTGAAGAACGGCAGGGAGTTCCGCGGCGAGCACCGC
ATTGGAGGCATCAAGCTGCGGCATCAGCAGTGGAGCCTGGTCATGGAAAG
CGTGGTGCCCTCGGACCGCGGCAACTACACCTGCGTCGTGGAGAACAAGT
TTGGCAGCATCCGGCAGACGTACACGCTGGACGTGCTGGAGCGCTCCCCG
CACCGGCCCATCCTGCAGGCGGGGCTGCCGGCCAACCAGACGGCGGTGCT
GGGCAGCGACGTGGAGTTCCACTGCAAGGTGTACAGTGACGCACAGCCCC
ACATCCAGTGGCTCAAGCACGTGGAGGTGAATGGCAGCAAGGTGGGCCCG
GACGGCACACCCTACGTTACCGTGCTCAAGACGGCGGGCGCTAACACCAC
CGACAAGGAGCTAGAGGTTCTCTCCTTGCACAACGTCACCTTTGAGGACG
CCGGGGAGTACACCTGCCTGGCGGGCAATTCTATTGGGTTTTCTCATCAC
TCTGCGTGGCTGGTGGTGCTGCCAGCCGAGGAGGAGCTGGTGGAGGCTGA
CGAGGCGGGCAGTGTGTATGCAGGCATCCTCAGCTACGGGGTGGGCTTCT
TCCTGTTCATCCTGGTGGTGGCGGCTGTGACGCTCTGCCGCCTGCGCAGC
CCCCCCAAGAAAGGCCTGGGCTCCCCCACCGTGCACAAGATCTCCCGCTT
CCCGCTCAAGCGACAGGTGTCCCTGGAGTCCAACGCGTCCATGAGCTCCA
ACACACCACTGGTGCGCATCGCAAGGCTGTCCTCAGGGGAGGGCCCCACG
CTGGCCAATGTCTCCGAGCTCGAGCTGCCTGCCGACCCCAAATGGGAGCT
GTCTCGGGCCCGGCTGACCCTGGGCAAGCCCCTTGGGGAGGGCTGCTTCG
GCCAGGTGGTCATGGCGGAGGCCATCGGCATTGACAAGGACCGGGCCGCC
AAGCCTGTCACCGTAGCCGTGAAGATGCTGAAAGACGATGCCACTGACAA
GGACCTGTCGGACCTGGTGTCTGAGATGGAGATGATGAAGATGATCGGGA
AACACAAAAACATCATCAACCTGCTGGGCGCCTGCACGCAGGGCGGGCCC
CTGTACGTGCTGGTGGAGTACGCGGCCAAGGGTAACCTGCGGGAGTTTCT
GCGGGCGCGGCGGCCCCCGGGCCTGGACTACTCCTTCGACACCTGCAAGC
CGCCCGAGGAGCAGCTCACCTTCAAGGACCTGGTGTCCTGTGCCTACCAG
GTGGCCCGGGGCATGGAGTACTTGGCCTCCCAGAAGTGCATCCACAGGGA
CCTGGCTGCCCGCAATGTGCTGGTGACCGAGGACAACGTGATGAAGATCG
CAGACTTCGGGCTGGCCCGGGACGTGCACAACCTCGACTACTACAAGAAG
ACGACCAACGGCCGGCTGCCCGTGAAGTGGATGGCGCCTGAGGCCTTGTT
TGACCGAGTCTACACTCACCAGAGTGACGTCTGGTCCTTTGGGGTCCTGC
TCTGGGAGATCTTCACGCTGGGGGGCTCCCCGTACCCCGGCATCCCTGTG
GAGGAGCTCTTCAAGCTGCTGAAGGAGGGCCACCGCATGGACAAGCCCGC
CAACTGCACACACGACCTGTACATGATCATGCGGGAGTGCTGGCATGCCG
CGCCCTCCCAGAGGCCCACCTTCAAGCAGCTGGTGGAGGACCTGGACCGT
GTCCTTACCGTGACGTCCACCGACGAGTACCTGGACCTGTCGGCGCCTTT
CGAGCAGTACTCCCCGGGTGGCCAGGACACCCCCAGCTCCAGCTCCTCAG
GGGACGTGCGTGAGCCACCGCACCCGGCGTTTCCTAGGTCAGCGGAGGAC
ACGCCTGTGGTGCAGTTGGCAGCCGAGACCCCAACAGCAGAGAGCAAGGA
GAGAGCCTTGAACTCTGCCAGCACCTCGCTTCCCACAAGCTGTCCAGGCA
GTGAGCCAGTGCCCACCCATCAGCAGGGGCAGCCTGCCTTGGAGCTGAAA
GAGGAGAGCTTCAGAGACCCCGCTGAGGTTCTAGGCACGGGCGCGGAGGT
GGATTACCTGGAGCAGTTTGGAACTTCCTCGTTTAAGGAGTCGGCCTTGA
GGAAGCAGTCCTTATACCTCAAGTTCGACCCCCTCCTGAGGGACAGTCCT
GGTAGACCAGTGCCCGTGGCCACCGAGACCAGCAGCATGCACGGTGCAAA
TGAGACTCCCTCAGGACGTCCGCGGGAAGCCAAGCTTGTGGAGTTCGATT
TCTTGGGAGCACTGGACATTCCTGTGCCAGGCCCACCCCCAGGTGTTCCC
GCGCCTGGGGGCCCACCCCTGTCCACCGGACCTATAGTGGACCTGCTCCA
GTACAGCCAGAAGGACCTGGATGCAGTGGTAAAGGCGACACAGGAGGAGA
ACCGGGAGCTGAGGAGCAGGTGTGAGGAGCTCCACGGGAAGAACCTGGAA
CTGGGGAAGATCATGGACAGGTTCGAAGAGGTTGTGTACCAGGCCATGGA
GGAAGTTCAGAAGCAGAAGGAACTTTCCAAAGCTGAAATCCAGAAAGTTC
TAAAAGAAAAAGACCAACTTACCACAGATCTGAACTCCATGGAGAAGTCC
TTCTCCGACCTCTTCAAGCGTTTTGAGAAACAGAAAGAGGTGATCGAGGG
CTACCGCAAGAACGAAGAGTCACTGAAGAAGTGCGTGGAGGATTACCTGG
CAAGGATCACCCAGGAGGGCCAGAGGTACCAAGCCCTGAAGGCCCACGCG
GAGGAGAAGCTGCAGCTGGCAAACGAGGAGATCGCCCAGGTCCGGAGCAA
GGCCCAGGCGGAAGCGTTGGCCCTCCAGGCCAGCCTGAGGAAGGAGCAGA
TGCGCATCCAGTCGCTGGAGAAGACAGTGGAGCAGAAGACTAAAGAGAAC
GAGGAGCTGACCAGGATCTGCGACGACCTCATCTCCAAGATGGAGAAGAT
CTGA

SEQ ID NO: 538 is the nucleotide sequence (cDNA) of FGFR3ex18-TACC3ex4. The sequence corresponding to FGFR3 is underlined. The sequence corresponding to TACC3 is italicized.

ATGGGCGCCCCTGCCTGCGCCCTCGCGCTCTGCGTGGCCGTGGCCATCGT
GGCCGGCGCCTCCTCGGAGTCCTTGGGGACGGAGCAGCGCGTCGTGGGGC
GAGCGGCAGAAGTCCCGGGCCCAGAGCCCGGCCAGCAGGAGCAGTTGGTC
TTCGGCAGCGGGGATGCTGTGGAGCTGAGCTGTCCCCCGCCCGGGGGTGG
TCCCATGGGGCCCACTGTCTGGGTCAAGGATGGCACAGGGCTGGTGCCCT
CGGAGCGTGTCCTGGTGGGGCCCCAGCGGCTGCAGGTGCTGAATGCCTCC
CACGAGGACTCCGGGGCCTACAGCTGCCGGCAGCGGCTCACGCAGCGCGT
ACTGTGCCACTTCAGTGTGCGGGTGACAGACGCTCCATCCTCGGGAGATG
ACGAAGACGGGGAGGACGAGGCTGAGGACACAGGTGTGGACACAGGGGCC
CCTTACTGGACACGGCCCGAGCGGATGGACAAGAAGCTGCTGGCCGTGCC
GGCCGCCAACACCGTCCGCTTCCGCTGCCCAGCCGCTGGCAACCCCACTC
CCTCCATCTCCTGGCTGAAGAACGGCAGGGAGTTCCGCGGCGAGCACCGC
ATTGGAGGCATCAAGCTGCGGCATCAGCAGTGGAGCCTGGTCATGGAAAG
CGTGGTGCCCTCGGACCGCGGCAACTACACCTGCGTCGTGGAGAACAAGT
TTGGCAGCATCCGGCAGACGTACACGCTGGACGTGCTGGAGCGCTCCCCG
CACCGGCCCATCCTGCAGGCGGGGCTGCCGGCCAACCAGACGGCGGTGCT
GGGCAGCGACGTGGAGTTCCACTGCAAGGTGTACAGTGACGCACAGCCCC
ACATCCAGTGGCTCAAGCACGTGGAGGTGAATGGCAGCAAGGTGGGCCCG
GACGGCACACCCTACGTTACCGTGCTCAAGACGGCGGGCGCTAACACCAC
CGACAAGGAGCTAGAGGTTCTCTCCTTGCACAACGTCACCTTTGAGGACG
CCGGGGAGTACACCTGCCTGGCGGGCAATTCTATTGGGTTTTCTCATCAC
TCTGCGTGGCTGGTGGTGCTGCCAGCCGAGGAGGAGCTGGTGGAGGCTGA
CGAGGCGGGCAGTGTGTATGCAGGCATCCTCAGCTACGGGGTGGGCTTCT
TCCTGTTCATCCTGGTGGTGGCGGCTGTGACGCTCTGCCGCCTGCGCAGC
CCCCCCAAGAAAGGCCTGGGCTCCCCCACCGTGCACAAGATCTCCCGCTT
CCCGCTCAAGCGACAGGTGTCCCTGGAGTCCAACGCGTCCATGAGCTCCA
ACACACCACTGGTGCGCATCGCAAGGCTGTCCTCAGGGGAGGGCCCCACG
CTGGCCAATGTCTCCGAGCTCGAGCTGCCTGCCGACCCCAAATGGGAGCT
GTCTCGGGCCCGGCTGACCCTGGGCAAGCCCCTTGGGGAGGGCTGCTTCG
GCCAGGTGGTCATGGCGGAGGCCATCGGCATTGACAAGGACCGGGCCGCC
AAGCCTGTCACCGTAGCCGTGAAGATGCTGAAAGACGATGCCACTGACAA
GGACCTGTCGGACCTGGTGTCTGAGATGGAGATGATGAAGATGATCGGGA
AACACAAAAACATCATCAACCTGCTGGGCGCCTGCACGCAGGGCGGGCCC
CTGTACGTGCTGGTGGAGTACGCGGCCAAGGGTAACCTGCGGGAGTTTCT
GCGGGCGCGGCGGCCCCCGGGCCTGGACTACTCCTTCGACACCTGCAAGC
CGCCCGAGGAGCAGCTCACCTTCAAGGACCTGGTGTCCTGTGCCTACCAG
GTGGCCCGGGGCATGGAGTACTTGGCCTCCCAGAAGTGCATCCACAGGGA
CCTGGCTGCCCGCAATGTGCTGGTGACCGAGGACAACGTGATGAAGATCG
CAGACTTCGGGCTGGCCCGGGACGTGCACAACCTCGACTACTACAAGAAG
ACGACCAACGGCCGGCTGCCCGTGAAGTGGATGGCGCCTGAGGCCTTGTT
TGACCGAGTCTACACTCACCAGAGTGACGTCTGGTCCTTTGGGGTCCTGC
TCTGGGAGATCTTCACGCTGGGGGGCTCCCCGTACCCCGGCATCCCTGTG
GAGGAGCTCTTCAAGCTGCTGAAGGAGGGCCACCGCATGGACAAGCCCGC
CAACTGCACACACGACCTGTACATGATCATGCGGGAGTGCTGGCATGCCG
CGCCCTCCCAGAGGCCCACCTTCAAGCAGCTGGTGGAGGACCTGGACCGT
GTCCTTACCGTGACGTCCACCGACGAGTACCTGGACCTGTCGGCGCCTTT
CGAGCAGTACTCCCCGGGTGGCCAGGACACCCCAGAAAGCCCTGAGACCA
GGCTGGGCCAGCCAGCGGCTGAACAGTTGCATGCTGGGCCTGCCACGGAG
GAGCCAGGTCCCTGTCTGAGCCAGCAGCTGCATTCAGCCTCAGCGGAGGA
CACGCCTGTGGTGCAGTTGGCAGCCGAGACCCCAACAGCAGAGAGCAAGG
AGAGAGCCTTGAACTCTGCCAGCACCTCGCTTCCCACAAGCTGTCCAGGC
GTGAGCCAGTGCCCACCCATCAGCAGGGGCAGCCTGCCTTGGAGCTGAA
AGAGGAGAGCTTCAGAGACCCCGCTGAGGTTCTAGGCACGGGCGCGGAGG
TGGATTACCTGGAGCAGTTTGGAACTTCCTCGTTTAAGGAGTCGGCCTTG
AGGAAGCAGTCCTTATACCTCAAGTTCGACCCCCTCCTGAGGGACAGTCC
TGGTAGACCAGTGCCCGTGGCCACCGAGACCAGCAGCATGCACGGTGCAA
ATGAGACTCCCTCAGGACGTCCGCGGGAAGCCAAGCTTGTGGAGTTCGAT
TTCTTGGGAGCACTGGACATTCCTGTGCCAGGCCCACCCCCAGGTGTTCC
CGCGCCTGGGGGCCCACCCCTGTCCACCGGACCTATAGTGGACCTGCTCC
AGTACAGCCAGAAGGACCTGGATGCAGTGGTAAAGGCGACACAGGAGGAG
AACCGGGAGCTGAGGAGCAGGTGTGAGGAGCTCCACGGGAAGAACCTGGA
ACTGGGGAAGATCATGGACAGGTTCGAAGAGGTTGTGTACCAGGCCATGG
AGGAAGTTCAGAAGCAGAAGGAACTTTCCAAAGCTGAAATCCAGAAAGTT
CTAAAAGAAAAAGACCAACTTACCACAGATCTGAACTCCATGGAGAAGTC
CTTCTCCGACCTCTTCAAGCGTTTTGAGAAACAGAAAGAGGTGATCGAGG
GCTACCGCAAGAACGAAGAGTCACTGAAGAAGTGCGTGGAGGATTACCTG
GCAAGGATCACCCAGGAGGGCCAGAGGTACCAAGCCCTGAAGGCCCACGC
GGAGGAGAAGCTGCAGCTGGCAAACGAGGAGATCGCCCAGGTCCGGAGCA
AGGCCCAGGCGGAAGCGTTGGCCCTCCAGGCCAGCCTGAGGAAGGAGCAG
ATGCGCATCCAGTCGCTGGAGAAGACAGTGGAGCAGAAGACTAAAGAGAA
CGAGGAGCTGACCAGGATCTGCGACGACCTCATCTCCAAGATGGAGAAGA
TCTGA

The Genbank ID for the FGFR1 gene is 2260. Eight isoforms are listed for FGFR1, e.g., having Genebank Accession Nos. NP_001167534 (corresponding nucleotide sequence NM_001174063); NP_001167535 (corresponding nucleotide sequence NM_001174064); NP_001167536 (corresponding nucleotide sequence NM_001174065); NP_001167537 (corresponding nucleotide sequence NM_001174066); NP_001167538 (corresponding nucleotide sequence NM_001174067); NP_056934 (corresponding nucleotide sequence NM_015850); NP_075593 (corresponding nucleotide sequence NM_023105); NP_075594 (corresponding nucleotide sequence NM_023106); NP_075598 (corresponding nucleotide sequence NM_023110).

SEQ ID NO: 146 is the FGFR1 Amino Acid Sequence for isoform 10, having Genebank Accession No. NP_001167534 (820 aa):

| | |
|---|---|
| 1 | MWSWKCLLFW AVLVTATLCT ARPSPTLPEQ AQPWGAPVEV ESFLVHPGDL LQLRCRLRDD |
| 61 | VQSINWLRDG VQLAESNRTR ITGEEVEVQD SVPADSGLYA CVTSSPSGSD TTYFSVNVSD |
| 121 | ALPSSEDDDD DDDSSSEEKE TDNTKPNRMP VAPYWTSPEK MEKKLHAVPA AKTVKFKCPS |
| 181 | SGTPNPTLRW LKNGKEFKPD HRIGGYKVRY ATWSIIMDSV VPSDKGNYTC IVENEYGSIN |
| 241 | HTYQLDVVER SPHRPILQAG LPANKTVALG SNVEFMCKVY SDPQPHIQWL KHIEVNGSKI |
| 301 | GPDNLPYVQI LKTAGVNTTD KEMEVLHLRN VSFEDAGEYT CLAGNSIGLS HHSAWLTVLE |
| 361 | ALEERPAVMT SPLYLEIIIY CTGAFLISCM VGSVIVYKMK SGTKKSDFHS QMAVHKLAKS |
| 421 | IPLRRQVSAD SSASMNSGVL LVRPSRLSSS GTPMLAGVSE YELPEDPRWE LPRDRLVLGK |
| 481 | PLGEGCFGQV VLAEAIGLDK DKPNRVTKVA VKMLKSDATE KDLSDLISEM EMMKMIGKHK |
| 541 | NIINLLGACT QDGPLYVIVE YASKGNLREY LQARRPPGLE YCYNPSHNPE EQLSSKDLVS |
| 601 | CAYQVARGME YLASKKCIHR DLAARNVLVT EDNVMKIADF GLARDIHHID YYKKTTNGRL |
| 661 | PVKWMAPEAL FDRIYTHQSD VWSFGVLLWE IFTLGGSPYP GVPVEELFKL LKEGHRMDKP |
| 721 | SNCTNELYMM MRDCWHAVPS QRPTFKQLVE DLDRIVALTS NQEYLDLSMP LDQYSPSFPD |
| 781 | TRSSTCSSGE DSVFSHEPLP EEPCLPRHPA QLANGGLKRR |

SEQ ID NO: 147 is the FGFR1 Nucleotide Sequence for isoform 10, having Genebank Accession No. NM_001174063 (5895 bp):

| | |
|---|---|
| 1 | agatgcaggg gcgcaaacgc caaaggagac caggctgtag gaagagaagg gcagagcgcc |
| 61 | ggacagctcg gcccgctccc cgtcctttgg ggccgcggct ggggaactac aaggcccagc |
| 121 | aggcagctgc agggggcgga ggcggaggag ggaccagcgc gggtgggagt gagagagcga |
| 181 | gccctcgcgc cccgccggcg catagcgctc ggagcgctct tgcggccaca ggcgcggcgt |
| 241 | cctcggcggc gggcggcagc tagcgggagc cgggacgccg gtgcagccgc agcgcgcgga |
| 301 | ggaacccggg tgtgccggga gctgggcggc cacgtccgga cgggaccgag acccctcgta |
| 361 | gcgcattgcg gcgacctcgc cttccccggc cgcgagcgcg ccgctgcttg aaaagccgcg |
| 421 | gaacccaagg acttttctcc ggtccgagct cggggcgccc cgcagggcgc acggtacccg |
| 481 | tgctgcagtc gggcacgccg cggcgccggg gcctccgcag ggcgatggag cccggtctgc |
| 541 | aaggaaagtg aggcgccgcc gctgcgttct ggaggagggg ggcacaaggt ctggagaccc |
| 601 | cgggtggcgg acgggagccc tccccccgcc ccgcctccgg ggcaccagct ccggctccat |
| 661 | tgttcccgcc cgggctggag gcgccgagca ccgagcgccg ccgggagtcg agcgccggcc |
| 721 | gcggagctct tgcgaccccg ccaggacccg aacagagccc gggggcggcg ggccggagcc |
| 781 | ggggacgcgg gcacacgccc gctcgcacaa gccacggcgg actctcccga ggcggaacct |
| 841 | ccacgccgag cgagggtcag tttgaaaagg aggatcgagc tcactgtgga gtatccatgg |
| 901 | agatgtggag ccttgtcacc aacctctaac tgcagaactg ggatgtggag ctggaagtgc |
| 961 | ctcctcttct gggctgtgct ggtcacagcc acactctgca ccgctaggcc gtccccgacc |
| 1021 | ttgcctgaac aagcccagcc ctggggagcc cctgtggaag tggagtcctt cctggtccac |
| 1081 | cccggtgacc tgctgcagct tcgctgtcgg ctgcgggacg atgtgcagag catcaactgg |
| 1141 | ctgcgggacg gggtgcagct ggcggaaagc aaccgcaccc gcatcacagg ggaggaggtg |
| 1201 | gaggtgcagg actccgtgcc cgcagactcc ggcctctatg cttgcgtaac cagcagcccc |
| 1261 | tcgggcagtg acaccaccta cttctccgtc aatgtttcag atgctctccc ctcctcggag |
| 1321 | gatgatgatg atgatgatga ctcctcttca gaggagaaag aaacagataa caccaaacca |
| 1381 | aaccgtatgc ccgtagctcc atattggaca tccccagaaa agatggaaaa gaaattgcat |
| 1441 | gcagtgccgg ctgccaagac agtgaagttc aaatgccctt ccagtgggac cccaaacccc |
| 1501 | acactgcgct ggttgaaaaa tggcaaagaa ttcaaacctg accacagaat tggaggctac |
| 1561 | aaggtccgtt atgccacctg gagcatcata atggactctg tggtgccctc tgacaagggc |
| 1621 | aactacacct gcattgtgga gaatgagtac ggcagcatca accacacata ccagctggat |
| 1681 | gtcgtggagc ggtcccctca ccggcccatc ctgcaagcag ggttgcccgc caacaaaaca |
| 1741 | gtggccctgg gtagcaacgt ggagttcatg tgtaaggtgt acagtgaccc gcagccgcac |
| 1801 | atccagtggc taaagcacat cgaggtgaat gggagcaaga ttggcccaga caacctgcct |
| 1861 | tatgtccaga tcttgaagac tgctggagtt aataccaccg acaaagagat ggaggtgctt |
| 1921 | cacttaagaa atgtctcctt tgaggacgca ggggagtata cgtgcttggc gggtaactct |
| 1981 | atcggactct cccatcactc tgcatggttg accgttctgg aagccctgga agagaggccg |
| 2041 | gcagtgatga cctcgcccct gtacctggag atcatcatct attgcacagg ggccttcctc |
| 2101 | atctcctgca tggtggggtc ggtcatcgtc tacaagatga agagtggtac caagaagagt |
| 2161 | gacttccaca gccagatggc tgtgcacaag ctggccaaga gcatccctct gcgcagacag |
| 2221 | gtgtctgctg actccagtgc atccatgaac tctggggttc ttctggttcg gccatcacgg |
| 2281 | ctctcctcca gtgggactcc catgctagca ggggtctctg agtatgagct tcccgaagac |
| 2341 | cctcgctggg agctgcctcg ggacagactg gtcttaggca aacccctggg agagggctgc |
| 2401 | tttgggcagg tggtgttggc agaggctatc gggctggaca aggacaaacc caaccgtgtg |
| 2461 | accaaagtgg ctgtgaagat gttgaagtcg gacgcaacag agaaagactt gtcagacctg |
| 2521 | atctcagaaa tggagatgat gaagatgatc gggaagcata agaatatcat caacctgctg |
| 2581 | ggggcctgca cgcaggatgg tcccttgtat gtcatcgtgg agtatgcctc caagggcaac |
| 2641 | ctgcgggagt acctgcaggc ccggaggccc ccagggctgg aatactgcta caaccccagc |
| 2701 | cacaacccag aggagcagct ctcctccaag gacctggtgt cctgcgccta ccaggtggcc |
| 2761 | cgaggcatgg agtatctggc ctccaagaag tgcatacacc gagacctggc agccaggaat |
| 2821 | gtcctggtga cagaggacaa tgtgatgaag atagcagact ttggcctcgc acgggacatt |
| 2881 | caccacatcg actactataa aaagacaacc aacggccgac tgcctgtgaa gtggatggca |
| 2941 | cccgaggcat tatttgaccg gatctacacc caccagagtg atgtgtggtc tttcggggtg |
| 3001 | ctcctgtggg agatcttcac tctgggcggc tccccatacc ccggtgtgcc tgtggaggaa |
| 3061 | cttttcaagc tgctgaagga gggtcaccgc atggacaagc ccagtaactg caccaacgag |
| 3121 | ctgtacatga tgatgcggga ctgctggcat gcagtgccct cacagagacc caccttcaag |
| 3181 | cagctggtgg aagacctgga ccgcatcgtg gccttgacct ccaaccagga gtacctggac |
| 3241 | ctgtccatgc ccctggacca gtactccccc agctttcccg acacccggag ctctacgtgc |
| 3301 | tcctcagggg aggattccgt cttctctcat gagccgctgc ccgaggagcc ctgcctgccc |
| 3361 | cgacacccag cccagcttgc caatggcgga ctcaaacgcc gctgactgcc acccacacgc |
| 3421 | cctccccaga ctccaccgtc agctgtaacc ctcacccaca gcccctgctg ggcccaccac |
| 3481 | ctgtccgtcc ctgtcccctt tcctgctggc aggagccggc tgcctaccag gggccttcct |
| 3541 | gtgtggcctg ccttcacccc actcagctca cctctccctc cacctcctct ccacctgctg |
| 3601 | gtgagaggtg caaagaggca gatctttgct gccagccact tcatcccctc ccagatgttg |
| 3661 | gaccaacacc cctccctgcc accaggcact gcctggaggg cagggagtgg gagccaatga |
| 3721 | acaggcatgc aagtgagagc ttcctgagct ttctcctgtc ggtttggtct gttttgcctt |
| 3781 | cacccataag cccctcgcac tctggtggca ggtgccttgt cctcagggct acagcagtag |
| 3841 | ggaggtcagt gcttcgtgcc tcgattgaag gtgacctctg ccccagatag gtggtgccag |
| 3901 | tggcttatta attccgatac tagtttgctt tgctgaccaa atgcctggta ccagaggatg |
| 3961 | gtgaggcgaa ggccaggttg ggggcagtgt tgtggccctg gggcccagcc ccaaactggg |
| 4021 | ggctctgtat atagctatga agaaaacaca aagtgtataa atctgagtat atatttacat |
| 4081 | gtctttttaa aagggtcgtt accagagatt tacccatcgg gtaagatgct cctggtggct |
| 4141 | gggaggcatc agttgctata tattaaaaac aaaaaagaaa aaaaaggaaa atgtttttaa |
| 4201 | aaaggtcata tattttttgc tacttttgct gttttatttt tttaaattat gttctaaacc |
| 4261 | tattttcagt ttaggtccct caataaaaat tgctgctgct tcatttatct atgggctgta |
| 4321 | tgaaaagggt gggaatgtcc actggaaaga agggacaccc acgggccctg gggctaggtc |
| 4381 | tgtcccgagg gcaccgcatg ctcccggcgc aggttccttg taacctcttc ttcctaggtc |
| 4441 | ctgcacccag acctcacgac gcacctcctg cctctccgct gcttttggaa agtcagaaaa |
| 4501 | agaagatgtc tgcttcgagg gcaggaaccc catccatgca gtagaggcgc tgggcagaga |
| 4561 | gtcaaggccc agcagccatc gaccatggat ggtttcctcc aaggaaaccg gtggggttgg |
| 4621 | gctggggagg gggcacctac ctaggaatag ccacggggta gagctacagt gattaagagg |
| 4681 | aaagcaaggg cgcggttgct cacgcctgta atcccagcac tttgggacac cgaggtgggc |
| 4741 | agatcacttc aggtcaggag tttgagacca gcctggccaa cttagtgaaa ccccatctct |
| 4801 | actaaaaatg caaaaattat ccaggcatgg tggcacacgc ctgtaatccc agctccacag |
| 4861 | gaggctgagg cagaatccct tgaagctggg aggcggaggt tgcagtgagc cgagattgcg |
| 4921 | ccattgcact ccagcctggg caacagagaa aacaaaaagg aaaacaaatg atgaaggtct |
| 4981 | gcagaaactg aaacccagac atgtgtctgc cccctctatg tgggcatggt tttgccagtg |
| 5041 | cttctaagtg caggagaaca tgtcacctga ggctagtttt gcattcaggt ccctggcttc |
| 5101 | gtttcttgtt ggtatgcctc cccagatcgt ccttcctgta tccatgtgac cagactgtat |
| 5161 | ttgttgggac tgtcgcagat cttggcttct tacagttctt cctgtccaaa ctccatcctg |
| 5221 | tccctcagga acggggggaa aattctccga atgtttttgg ttttttggct gcttggaatt |
| 5281 | tacttctgcc acctgctggt catcactgtc ctcactaagt ggattctggc tcccccgtac |
| 5341 | ctcatggctc aaactaccac tcctcagtcg ctatattaaa gcttatattt tgctggatta |
| 5401 | ctgctaaata caaaagaaag ttcaatatgt tttcatttct gtagggaaaa tgggattgct |
| 5461 | gctttaaatt tctgagctag ggattttttg gcagctgcag tgttggcgac tattgtaaaa |
| 5521 | ttctctttgt ttctctctgt aaatagcacc tgctaacatt acaatttgta tttatgttta |
| 5581 | aagaaggcat catttggtga acagaactag gaaatgaatt tttagctctt aaaagcattt |
| 5641 | gctttgagac cgcacaggag tgtctttcct tgtaaaacag tgatgataat ttctgccttg |
| 5701 | gccctacctt gaagcaatgt tgtgtgaagg gatgaagaat ctaaaagtct tcataagtcc |
| 5761 | ttgggagagg tgctagaaaa atataaggca ctatcataat tacagtgatg tccttgctgt |
| 5821 | tactactcaa atcacccaca aatttcccca aagactgcgc tagctgtcaa ataaaagaca |
| 5881 | gtgaaattga cctga |

SEQ ID NO: 185 is the FGFR1 Amino Acid Sequence for isoform 1, having Genebank Accession No. NP_075598 (822 aa):

| | |
|---|---|
| 1 | MWSWKCLLFW AVLVTATLCT ARPSPTLPEQ AQPWGAPVEV ESFLVHPGDL LQLRCRLRDD |
| 61 | VQSINWLRDG VQLAESNRTR ITGEEVEVQD SVPADSGLYA CVTSSPSGSD TTYFSVNVSD |
| 121 | ALPSSEDDDD DDDSSSEEKE TDNTKPNRMP VAPYWTSPEK MEKKLHAVPA AKTVKFKCPS |
| 181 | SGTPNPTLRW LKNGKEFKPD HRIGGYKVRY ATWSIIMDSV VPSDKGNYTC IVENEYGSIN |
| 241 | HTYQLDVVER SPHRPILQAG LPANKTVALG SNVEFMCKVY SDPQPHIQWL KHIEVNGSKI |
| 301 | GPDNLPYVQI LKTAGVNTTD KEMEVLHLRN VSFEDAGEYT CLAGNSIGLS HHSAWLTVLE |
| 361 | ALEERPAVMT SPLYLEIIIY CTGAFLISCM VGSVIVYKMK SGTKKSDFHS QMAVHKLAKS |
| 421 | IPLRRQVTVS ADSSASMNSG VLLVRPSRLS SSGTPMLAGV SEYELPEDPR WELPRDRLVL |
| 481 | GKPLGEGCFG QVVLAEAIGL DKDKPNRVTK VAVKMLKSDA TEKDLSDLIS EMEMMKMIGK |
| 541 | HKNIINLLGA CTQDGPLYVI VEYASKGNLR EYLQARRPPG LEYCYNPSHN PEEQLSSKDL |
| 601 | VSCAYQVARG MEYLASKKCI HRDLAARNVL VTEDNVMKIA DFGLARDIHH IDYYKKTTNG |
| 661 | RLPVKWMAPE ALFDRIYTHQ SDVWSFGVLL WEIFTLGGSP YPGVPVEELF KLLKEGHRMD |
| 721 | KPSNCTNELY MMMRDCWHAV PSQRPTFKQL VEDLDRIVAL TSNQEYLDLS MPLDQYSPSF |
| 781 | PDTRSSTCSS GEDSVFSHEP LPEEPCLPRH PAQLANGGLK RR |

SEQ ID NO: 186 is the FGFR1 Nucleotide Sequence for isoform 1, having Genebank Accession No. NM_023110 (5917 bp):

| | |
|---|---|
| 1 | agatgcaggg gcgcaaacgc caaaggagac caggctgtag gaagagaagg gcagagcgcc |
| 61 | ggacagctcg gcccgctccc cgtcctttgg ggccgcggct ggggaactac aaggcccagc |
| 121 | aggcagctgc agggggcgga ggcggaggag ggaccagcgc gggtgggagt gagagagcga |
| 181 | gccctcgcgc cccgccggcg catagcgctc ggagcgctct tgcggccaca ggcgcggcgt |
| 241 | cctcggcggc gggcggcagc tagcgggagc cgggacgccg gtgcagccgc agcgcgcgga |
| 301 | ggaacccggg tgtgccggga gctgggcggc cacgtccgga cgggaccgag acccctcgta |
| 361 | gcgcattgcg gcgacctcgc cttccccggc cgcgagcgcg ccgctgcttg aaaagccgcg |
| 421 | gaacccaagg acttttctcc ggtccgagct cggggcgccc cgcagggcgc acggtacccg |
| 481 | tgctgcagtc gggcacgccg cggcgccggg gcctccgcag ggcgatggag cccggtctgc |
| 541 | aaggaaagtg aggcgccgcc gctgcgttct ggaggagggg ggcacaaggt ctggagaccc |
| 601 | cgggtggcgg acgggagccc tccccccgcc ccgcctccgg ggcaccagct ccggctccat |
| 661 | tgttcccgcc cgggctggag gcgccgagca ccgagcgccg ccgggagtcg agcgccggcc |
| 721 | gcggagctct tgcgaccccg ccaggacccg aacagagccc gggggcggcg ggccggagcc |
| 781 | ggggacgcgg gcacacgccc gctcgcacaa gccacggcgg actctcccga ggcggaacct |
| 841 | ccacgccgag cgagggtcag tttgaaaagg aggatcgagc tcactgtgga gtatccatgg |
| 901 | agatgtggag ccttgtcacc aacctctaac tgcagaactg ggatgtggag ctggaagtgc |
| 961 | ctcctcttct gggctgtgct ggtcacagcc acactctgca ccgctaggcc gtccccgacc |
| 1021 | ttgcctgaac aagcccagcc ctggggagcc cctgtggaag tggagtcctt cctggtccac |
| 1081 | cccggtgacc tgctgcagct tcgctgtcgg ctgcgggacg atgtgcagag catcaactgg |
| 1141 | ctgcgggacg gggtgcagct ggcggaaagc aaccgcaccc gcatcacagg ggaggaggtg |
| 1201 | gaggtgcagg actccgtgcc cgcagactcc ggcctctatg cttgcgtaac cagcagcccc |
| 1261 | tcgggcagtg acaccaccta cttctccgtc aatgtttcag atgctctccc ctcctcggag |
| 1321 | gatgatgatg atgatgatga ctcctcttca gaggagaaag aaacagataa caccaaacca |
| 1381 | aaccgtatgc ccgtagctcc atattggaca tccccagaaa agatggaaaa gaaattgcat |
| 1441 | gcagtgccgg ctgccaagac agtgaagttc aaatgccctt ccagtgggac cccaaacccc |
| 1501 | acactgcgct ggttgaaaaa tggcaaagaa ttcaaacctg accacagaat tggaggctac |
| 1561 | aaggtccgtt atgccacctg gagcatcata atggactctg tggtgccctc tgacaagggc |
| 1621 | aactacacct gcattgtgga gaatgagtac ggcagcatca accacacata ccagctggat |
| 1681 | gtcgtggagc ggtcccctca ccggcccatc ctgcaagcag ggttgcccgc caacaaaaca |
| 1741 | gtggccctgg gtagcaacgt ggagttcatg tgtaaggtgt acagtgaccc gcagccgcac |
| 1801 | atccagtggc taaagcacat cgaggtgaat gggagcaaga ttggcccaga caacctgcct |
| 1861 | tatgtccaga tcttgaagac tgctggagtt aataccaccg acaaagagat ggaggtgctt |
| 1921 | cacttaagaa atgtctcctt tgaggacgca ggggagtata cgtgcttggc gggtaactct |
| 1981 | atcggactct cccatcactc tgcatggttg accgttctgg aagccctgga agagaggccg |
| 2041 | gcagtgatga cctcgcccct gtacctggag atcatcatct attgcacagg ggccttcctc |
| 2101 | atctcctgca tggtggggtc ggtcatcgtc tacaagatga agagtggtac caagaagagt |
| 2161 | gacttccaca gccagatggc tgtgcacaag ctggccaaga gcatccctct gcgcagacag |
| 2221 | gtaacagtgt ctgctgactc cagtgcatcc atgaactctg gggttcttct ggttcggcca |
| 2281 | tcacggctct cctccagtgg gactcccatg ctagcagggg tctctgagta tgagcttccc |
| 2341 | gaagaccctc gctgggagct gcctcgggac agactggtct taggcaaacc cctgggagag |
| 2401 | ggctgctttg ggcaggtggt gttggcagag gctatcgggc tggacaagga caaacccaac |
| 2461 | cgtgtgacca aagtggctgt gaagatgttg aagtcggacg caacagagaa agacttgtca |
| 2521 | gacctgatct cagaaatgga gatgatgaag atgatcggga agcataagaa tatcatcaac |
| 2581 | ctgctggggg cctgcacgca ggatggtccc ttgtatgtca tcgtggagta tgcctccaag |
| 2641 | ggcaacctgc gggagtacct gcaggcccgg aggcccccag ggctggaata ctgctacaac |
| 2701 | cccagccaca acccagagga gcagctctcc tccaaggacc tggtgtcctg cgcctaccag |
| 2761 | gtggcccgag gcatggagta tctggcctcc aagaagtgca tacaccgaga cctggcagcc |
| 2821 | aggaatgtcc tggtgacaga ggacaatgtg atgaagatag cagactttgg cctcgcacgg |
| 2881 | gacattcacc acatcgacta ctataaaaag acaaccaacg gccgactgcc tgtgaagtgg |
| 2941 | atggcacccg aggcattatt tgaccggatc tacacccacc agagtgatgt gtggtctttc |
| 3001 | ggggtgctcc tgtgggagat cttcactctg ggcggctccc cataccccgg tgtgcctgtg |
| 3061 | gaggaacttt tcaagctgct gaaggagggt caccgcatgg acaagcccag taactgcacc |
| 3121 | aacgagctgt acatgatgat gcgggactgc tggcatgcag tgccctcaca gagacccacc |
| 3181 | ttcaagcagc tggtggaaga cctggaccgc atcgtggcct tgacctccaa ccaggagtac |
| 3241 | ctggacctgt ccatgcccct ggaccagtac tcccccagct ttcccgacac ccggagctct |
| 3301 | acgtgctcct caggggagga ttccgtcttc tctcatgagc cgctgcccga ggagccctgc |
| 3361 | ctgccccgac acccagccca gcttgccaat ggcggactca aacgccgctg actgccaccc |
| 3421 | acacgccctc cccagactcc accgtcagct gtaaccctca cccacagccc ctgctgggcc |
| 3481 | caccacctgt ccgtccctgt cccctttcct gctggcagga gccggctgcc taccaggggc |
| 3541 | cttcctgtgt ggcctgcctt caccccactc agctcacctc tccctccacc tcctctccac |
| 3601 | ctgctggtga gaggtgcaaa gaggcagatc tttgctgcca gccacttcat cccctcccag |
| 3661 | atgttggacc aacacccctc cctgccacca ggcactgcct ggagggcagg gagtgggagc |
| 3721 | caatgaacag gcatgcaagt gagagcttcc tgagctttct cctgtcggtt tggtctgttt |
| 3781 | tgccttcacc cataagcccc tcgcactctg gtggcaggtg ccttgtcctc agggctacag |
| 3841 | cagtagggag gtcagtgctt cgtgcctcga ttgaaggtga cctctgcccc agataggtgg |
| 3901 | tgccagtggc ttattaattc cgatactagt ttgctttgct gaccaaatgc ctggtaccag |
| 3961 | aggatggtga ggcgaaggcc aggttggggg cagtgttgtg gccctggggc ccagccccaa |
| 4021 | actgggggct ctgtatatag ctatgaagaa aacacaaagt gtataaatct gagtatatat |
| 4081 | ttacatgtct ttttaaaagg gtcgttacca gagatttacc catcgggtaa gatgctcctg |
| 4141 | gtggctggga ggcatcagtt gctatatatt aaaaacaaaa aagaaaaaaa aggaaaatgt |
| 4201 | ttttaaaaag gtcatatatt ttttgctact tttgctgttt tattttttta aattatgttc |
| 4261 | taaacctatt ttcagtttag gtccctcaat aaaaattgct gctgcttcat ttatctatgg |
| 4321 | gctgtatgaa aagggtggga atgtccactg gaaagaaggg acacccacgg gccctggggc |
| 4381 | taggtctgtc ccgagggcac cgcatgctcc cggcgcaggt tccttgtaac ctcttcttcc |
| 4441 | taggtcctgc acccagacct cacgacgcac ctcctgcctc tccgctgctt ttggaaagtc |
| 4501 | agaaaaagaa gatgtctgct tcgagggcag gaaccccatc catgcagtag aggcgctggg |
| 4561 | cagagagtca aggcccagca gccatcgacc atggatggtt tcctccaagg aaaccggtgg |
| 4621 | ggttgggctg gggagggggc acctacctag gaatagccac ggggtagagc tacagtgatt |
| 4681 | aagaggaaag caagggcgcg gttgctcacg cctgtaatcc cagcactttg ggacaccgag |
| 4741 | gtgggcagat cacttcaggt caggagtttg agaccagcct ggccaactta gtgaaacccc |
| 4801 | atctctacta aaaatgcaaa aattatccag gcatggtggc acacgcctgt aatcccagct |
| 4861 | ccacaggagg ctgaggcaga atcccttgaa gctgggaggc ggaggttgca gtgagccgag |
| 4921 | attgcgccat tgcactccag cctgggcaac agagaaaaca aaaaggaaaa caaatgatga |
| 4981 | aggtctgcag aaactgaaac ccagacatgt gtctgccccc tctatgtggg catggttttg |
| 5041 | ccagtgcttc taagtgcagg agaacatgtc acctgaggct agttttgcat tcaggtccct |
| 5101 | ggcttcgttt cttgttggta tgcctcccca gatcgtcctt cctgtatcca tgtgaccaga |
| 5161 | ctgtatttgt tgggactgtc gcagatcttg gcttcttaca gttcttcctg tccaaactcc |
| 5221 | atcctgtccc tcaggaacgg ggggaaaatt ctccgaatgt ttttggtttt ttggctgctt |
| 5281 | ggaatttact tctgccacct gctggtcatc actgtcctca ctaagtggat tctggctccc |
| 5341 | ccgtacctca tggctcaaac taccactcct cagtcgctat attaaagctt atattttgct |
| 5401 | ggattactgc taaatacaaa agaaagttca atatgttttc atttctgtag ggaaaatggg |
| 5461 | attgctgctt taaatttctg agctagggat tttttggcag ctgcagtgtt ggcgactatt |
| 5521 | gtaaaattct ctttgtttct ctctgtaaat agcacctgct aacattacaa tttgtattta |
| 5581 | tgtttaaaga aggcatcatt tggtgaacag aactaggaaa tgaattttta gctcttaaaa |
| 5641 | gcatttgctt tgagaccgca caggagtgtc tttccttgta aaacagtgat gataatttct |
| 5701 | gccttggccc taccttgaag caatgttgtg tgaagggatg aagaatctaa aagtcttcat |
| 5761 | aagtccttgg gagaggtgct agaaaaatat aaggcactat cataattaca gtgatgtcct |
| 5821 | tgctgttact actcaaatca cccacaaatt tccccaaaga ctgcgctagc tgtcaaataa |
| 5881 | aagacagtga aattgacctg aaaaaaaaaa aaaaaaa |

The Genbank ID for the TACC1 gene is 6867. Three isoforms are listed for TACC1, e.g., having Genebank Accession Nos. NP_006274 (corresponding nucleotide sequence NM_001174063); NP_001167535 (corresponding nucleotide sequence NM_001174064); NP_001167536 (corresponding nucleotide sequence NM_001174065).

SEQ ID NO: 148 is the TACC1 Amino Acid Sequence for isoform 1, having Genebank Accession No. NP_006274 (805 aa):

| | |
|---|---|
| 1 | MAFSPWQILS PVQWAKWTWS AVRGGAAGED EAGGPEGDPE EEDSQAETKS LSFSSDSEGN |
| 61 | FETPEAETPI RSPFKESCDP SLGLAGPGAK SQESQEADEQ LVAEVVEKCS SKTCSKPSEN |
| 121 | EVPQQAIDSH SVKNFREEPE HDFSKISIVR PFSIETKDST DISAVLGTKA AHGCVTAVSG |
| 181 | KALPSSPPDA LQDEAMTEGS MGVTLEASAE ADLKAGNSCP ELVPSRRSKL RKPKPVPLRK |
| 241 | KAIGGEFSDT NAAVEGTPLP KASYHFSPEE LDENTSPLLG DARFQKSPPD LKETPGTLSS |
| 301 | DTNDSGVELG EESRSSPLKL EFDFTEDTGN IEARKALPRK LGRKLGSTLT PKIQKDGISK |
| 361 | SAGLEQPTDP VARDGPLSQT SSKPDPSQWE SPSFNPFGSH SVLQNSPPLS SEGSYHFDPD |
| 421 | NFDESMDPFK PTTTLTSSDF CSPTGNHVNE ILESPKKAKS RLITSGCKVK KHETQSLALD |
| 481 | ACSRDEGAVI SQISDISNRD GHATDEEKLA STSCGQKSAG AEVKGEPEED LEYFECSNVP |
| 541 | VSTINHAFSS SEAGIEKETC QKMEEDGSTV LGLLESSAEK APVSVSCGGE SPLDGICLSE |
| 601 | SDKTAVLTLI REEIITKEIE ANEWKKKYEE TRQEVLEMRK IVAEYEKTIA QMIEDEQRTS |
| 661 | MTSQKSFQQL TMEKEQALAD LNSVERSLSD LFRRYENLKG VLEGFKKNEE ALKKCAQDYL |
| 721 | ARVKQEEQRY QALKIHAEEK LDKANEEIAQ VRTKAKAESA ALHAGLRKEQ MKVESLERAL |
| 781 | QQKNQEIEEL TKICDELIAK LGKTD |

SEQ ID NO: 149 is the TACC1 Nucleotide Sequence for isoform 1, having Genebank Accession No. NM_006283 (7802 bp):

| | |
|---|---|
| 1 | agctgatgcg cgccccgccg gccgggaggc gggagtccgc gagccgggag cgggagcagc |
| 61 | agaggtctag cagccgggcg ccgcgggccg ggggcctgag gaggccacag gacgggcgtc |
| 121 | ttcccggcta gtggagcccg gcgcggggcc cgctgcggcc gcaccgtgag gggaggaggc |
| 181 | cgaggaggac gcagcgccgg ctgccggcgg gaggaagcgc tccaccaggg cccccgacgg |
| 241 | cactcgttta accacatccg cgcctctgct ggaaacgctt gctggcgcct gtcaccggtt |
| 301 | ccctccattt tgaaagggaa aaaggctctc cccacccatt cccctgcccc taggagctgg |
| 361 | agccggagga gccgcgctca tggcgttcag cccgtggcag atcctgtccc ccgtgcagtg |
| 421 | ggcgaaatgg acgtggtctg cggtacgcgg cggggccgcc ggcgaggacg aggctggcgg |
| 481 | gcccgagggc gaccccgagg aggaggattc gcaagccgag accaaatcct tgagtttcag |
| 541 | ctcggattct gaaggtaatt ttgagactcc tgaagctgaa accccgatcc gatcaccttt |
| 601 | caaggagtcc tgtgatccat cactcggatt ggcaggacct ggggccaaaa gccaagaatc |
| 661 | acaagaagct gatgaacagc ttgtagcaga agtggttgaa aaatgttcat ctaagacttg |
| 721 | ttctaaacct tcagaaaatg aagtgccaca gcaggccatt gactctcact cagtcaagaa |
| 781 | tttcagagaa gaacctgaac atgattttag caaaatttcc atcgtgaggc cattttcaat |
| 841 | agaaacgaag gattccacgg atatctcggc agtcctcgga acaaaagcag ctcatggctg |
| 901 | tgtaactgca gtctcaggca aggctctgcc ttccagcccg ccagacgccc tccaggacga |
| 961 | ggcgatgaca gaaggcagca tgggggtcac cctcgaggcc tccgcagaag ctgatctaaa |
| 1021 | agctggcaac tcctgtccag agcttgtgcc cagcagaaga agcaagctga gaaagcccaa |
| 1081 | gcctgtcccc ctgaggaaga aagcaattgg aggagagttc tcagacacca acgctgctgt |
| 1141 | ggagggcaca cctctcccca aggcatccta tcacttcagt cctgaagagt tggatgagaa |
| 1201 | cacaagtcct ttgctaggag atgccaggtt ccagaagtct ccccctgacc ttaaagaaac |
| 1261 | tcccggcact ctcagtagtg acaccaacga ctcaggggtt gagctggggg aggagtcgag |
| 1321 | gagctcacct ctcaagcttg agtttgattt cacagaagat acaggaaaca tagaggccag |
| 1381 | gaaagccctt ccaaggaagc ttggcaggaa actgggtagc acactgactc ccaagataca |
| 1441 | aaaagatggc atcagtaagt cagcaggttt agaacagcct acagacccag tggcacgaga |
| 1501 | cgggcctctc tcccaaacat cttccaagcc agatcctagt cagtgggaaa gccccagctt |
| 1561 | caaccccttt gggagccact ctgttctgca gaactcccca cccctctctt ctgagggctc |
| 1621 | ctaccacttt gacccagata actttgacga atccatggat ccctttaaac caactacgac |
| 1681 | cttaacaagc agtgactttt gttctcccac tggtaatcac gttaatgaaa tcttagaatc |
| 1741 | acccaagaag gcaaagtcgc gtttaataac gagtggctgt aaggtgaaga agcatgaaac |
| 1801 | tcagtctctc gccctggatg catgttctcg ggatgaaggg gcagtgatct cccagatttc |
| 1861 | agacatttct aatagggatg gccatgctac tgatgaggag aaactggcat ccacgtcatg |
| 1921 | tggtcagaaa tcagctggtg ccgaggtgaa aggtgagcca gaggaagacc tggagtactt |
| 1981 | tgaatgttcc aatgttcctg tgtctaccat aaatcatgcg ttttcatcct cagaagcagg |
| 2041 | catagagaag gagacgtgcc agaagatgga agaagacggg tccactgtgc ttgggctgct |
| 2101 | ggagtcctct gcagagaagg cccctgtgtc ggtgtcctgt ggaggtgaga gccccctgga |
| 2161 | tgggatctgc ctcagcgaat cagacaagac agccgtgctc accttaataa gagaagagat |
| 2221 | aattactaaa gagattgaag caaatgaatg gaagaagaaa tacgaagaga cccggcaaga |
| 2281 | agttttggag atgaggaaaa ttgtagctga atatgaaaag actattgctc aaatgattga |
| 2341 | agatgaacaa aggacaagta tgacctctca gaagagcttc cagcaactga ccatggagaa |
| 2401 | ggaacaggcc ctggctgacc ttaactctgt ggaaaggtcc ctttctgatc tcttcaggag |
| 2461 | atatgagaac ctgaaaggtg ttctggaagg gttcaagaag aatgaagaag ccttgaagaa |
| 2521 | atgtgctcag gattacttag ccagagttaa acaagaggag cagcgatacc aggccctgaa |
| 2581 | aatccacgca gaagagaaac tggacaaagc caatgaagag attgctcagg ttcgaacaaa |
| 2641 | agcaaaggct gagagtgcag ctctccatgc tggactccgc aaagagcaga tgaaggtgga |
| 2701 | gtccctggaa agggccctgc agcagaagaa ccaagaaatt gaagaactga caaaaatctg |
| 2761 | tgatgagctg attgcaaagc tgggaaagac tgactgagac actccccctg ttagctcaac |
| 2821 | agatctgcat ttggctgctt ctcttgtgac cacaattatc ttgccttatc caggaataat |
| 2881 | tgcccctttg cagagaaaaa aaaaaactta aaaaaagcac atgcctactg ctgcctgtcc |
| 2941 | cgctttgctg ccaatgcaac agccctggaa gaaaccctag agggttgcat agtctagaaa |
| 3001 | ggagtgtgac ctgacagtgc tggagcctcc tagtttcccc ctatgaaggt tcccttaggc |
| 3061 | tgctgagttt gggtttgtga tttatcttta gtttgtttta aagtcatctt tactttccca |
| 3121 | aatgtgttaa atttgtaact cctctttggg gtcttctcca ccacctgtct gatttttttg |
| 3181 | tgatctgttt aatcttttaa ttttttagta tcagtggttt tatttaagga gacagtttgg |
| 3241 | cctattgtta cttccaattt ataatcaaga aggggctctg gatccccttt taaattacac |
| 3301 | acactctcac acacatacat gtatgtttat agatgctgct gctcttttcc ctgaagcata |
| 3361 | gtcaagtaag aactgctcta cagaaggaca tatttccttg gatgtgagac cctattttga |
| 3421 | aatagagtcc tgactcagaa caccaactta agaatttggg ggattaaaga tgtgaagacc |
| 3481 | acagtcttgg gttttcatat ctggagaaga ctatttgcca tgacgttttg ttgccctggt |
| 3541 | atttggacac tcctcagctt taatgggtgt ggccccttta gggttagtcc tcagactaat |
| 3601 | gatagtgtct gctttctgca tgaacggcaa tatgggactc cctccaagct agggtttggc |
| 3661 | aagtctgccc tagagtcatt tactctcctc tgcctccatt tgttaataca gaatcaacat |
| 3721 | ttagtcttca ttatcttttt tttttttttt gagacagagt ttcgatctat tttaagtatg |
| 3781 | tgaagaaaat ctacttgtaa aaggctcaga tcttaattaa aaggtaattg tagcacatta |
| 3841 | ccaattataa ggtgaagaaa tgtttttttc ccaagtgtga tgcattgttc ttcagatgtt |
| 3901 | gaaaagaaag caaaaaatac cttctaactt aagacagaat ttttaacaaa atgagcagta |
| 3961 | aaagtcacat gaaccactcc aaaaatcagt gcattttgca tatttttaaa caaagacagc |
| 4021 | ttgttgaata ctgagaagag gagtgcaagg agaaggtctg tactaacaaa gccaaattcc |
| 4081 | tcaagctctt actggactca gttcagagtg gtgggccatt aaccccaaca tggaattttt |
| 4141 | ccatataaat ctcaatgaat tccctttcat ttgaataggc aaacccaaat ccatgcaagt |
| 4201 | gttttaaagc actgtcctgt cttaatctta catgctgaaa gtcttcatgg tgatatgcac |
| 4261 | tatattcagt atacgtatgt tttcctactt ctcttgtaaa actgttgcat gatccaactt |
| 4321 | cagcaatgaa ttgtgcctag tggagaacct ctatagatct taaaaaatga attattcttt |
| 4381 | agcagtgtat tactcacatg ggtgcaatct ttagccccag ggaggtcaat aatgtctttt |
| 4441 | aaagccagaa gtcacatttt accaatatgc atttatcata attggtgctt aggctgtata |
| 4501 | ttcaagcctg ttgtcttaac attttgtata aaaaagaaca acagaaatta tctgtcattt |
| 4561 | gagaagtggc ttgacaatca tttgagcttt gaaagcagtc actgtggtgt aatatgaatg |
| 4621 | ctgtcctagt ggtcatagta ccaagggcac gtgtctcccc ttggtataac tgatttcctt |
| 4681 | tttagtcctc tactgctaaa taagttaatt ttgcattttg cagaaagaaa cattgattgc |
| 4741 | taaatctttt tgctgctgtg ttttggtgtt ttcatgttta cttgttttat attgatctgt |
| 4801 | tttaagtatg agaggcttat agtgccctcc attgtaaatc catagtcatc tttttaagct |
| 4861 | tattgtgttt aagaaagtag ctatgtgtta aacagaggtg atggcagccc ttccctagca |
| 4921 | cactggtgga agagacccct taagaacctg accccagtga atgaagctga tgcacaggga |
| 4981 | gcaccaaagg accttcgtta agtgataatt gtcctggcct ctcagccatg accgttatga |
| 5041 | ggaaatatcc cccattcgaa cttaacagat gcctcctctc caaagagaat taaaatcgta |
| 5101 | gcttgtacag atcaagagaa tatactgggc agaatgaagt atgtttgttt atttttcttt |
| 5161 | aaaaataaag gattttggaa ctctggagag taagaatata gtatagagtt tgcctcaaca |
| 5221 | catgtgaggg ccaaataacc tgctagctag gcagtaataa actctgttac agaagagaaa |
| 5281 | aagggccggg cacagtggct tattcctgta atcccaacac tgtggaaggc cgaggcagga |
| 5341 | ggatcacttg agtccaggag tttgaaacct acctaggcaa catggtgaaa ccttgtctct |
| 5401 | accaaaataa aaattagctg ggcatggtgg cacgtgcctg tggtcccagc tacttgggag |
| 5461 | gctgaggtgg gagcctggga ggtcaaggct gcagtgagcc atgatcatgc cactgcactc |
| 5521 | catcctgggt gacagcaaga tcttgtctca aaaaaaaaaa aaaaaaaaaa aaaaccagga |
| 5581 | gtgaaaaagg aaagtagaag gcagctgctg gcctagatgt tggtttggga atattaggtg |
| 5641 | atcctgttga gattctggat ccagagcaat ttctttagct tttgactttg ccaaagtgta |
| 5701 | gatagccttt atccagcagt attttaagtg gggaatgcaa cgtgaggcca actgaacaat |
| 5761 | tccccccgtg gctgcccaga tagtcacagt caaggttgga gagtctcctt ccagccagtg |
| 5821 | acctacccaa accttttgtt ctgtaaaact gctctggaaa taccgggaag cccagttttc |
| 5881 | tcacgtggtt tctagcttct tcagactcag cccaaattag gaagtgcaga agcacatgat |
| 5941 | ggtgaaaaac ctaggatttg gcagccttcc agaatggtat ggaatctgag ggaagattta |
| 6001 | tgtttcgttt tggaggatag ctcaagttga attttctttc cagccagtta ccctttcaac |
| 6061 | ctacccatac tttgtacaac tcttacacaa atacttagat atttattaga tagccctgaa |
| 6121 | ttcactctaa ttataaacag ggagtgtaaa ctgcccccag atgttcctgg gctgggtaaa |
| 6181 | agcagctgga gtgaagcact cattttccat aaaggtaaca aagggcagct cagtggttac |
| 6241 | tcaagctcaa aagggttttt ttaagagcaa gcattggtta agtctgtgta tactgagttg |
| 6301 | gaagtgattt cagcacattc ttttttagtg gagtgaaagt tctgaagccc ccttttaact |
| 6361 | tcctcttggt ttttcattat aattggtagc catctcatga actgtctctg actgttgtct |
| 6421 | ctttgtggtc atgtgattgt gagcttgctt tctgacttgc atttctgact ttatcctgtt |
| 6481 | gttaggaaga tagaaactag gttttgaaag attacatgat tcaagcgagg gattttaaag |
| 6541 | taaagatgta tttattctga agaatctaaa agataacaga ttatttgctt atgaaagaac |
| 6601 | aatatagtct gggaatccca gaatgtcaag ccaaaggtct aagaagtcat ctccttcaaa |
| 6661 | tactttaata aagaagtatt tcgaggagat atctgtccaa aaaggtttga ctggcctcca |
| 6721 | gattccagtt atttttaaaa agcaacttac cactaaatcc ttgagtctcc atagagtaac |
| 6781 | agtaaagaaa ctgatgtaac agactctcct ctcaaaggat ctcctctgga agagactatc |
| 6841 | agcggcagca ttctccaggg aagacccatc ccctagtgcc agagcttgca tcctggagac |
| 6901 | taaagattgc acttttttgt agttttttgt ccaaatgcaa tcccatttct gtgcctctta |
| 6961 | gcatgcagtt agatttggac aaacaagatt cctaaggaat gactttatta actataatat |
| 7021 | ggttacagct attatataaa tatatattct ggttatagtt ctaatatgga gatgttgtgt |
| 7081 | gcaatgctgg cctgtggtgg tctgtgtaat gctttaactt gtatggagga ggccaggctc |
| 7141 | agagctgaga tgtggcctga accttccctg tatcgatcct ttaatttaga actgtcaaga |
| 7201 | tgtcactttc tccccctctg ccttttagtg gtatctgaca tatactcaaa acagtaattt |
| 7261 | cctggtcaca tcattaactg ctaattctgt atttataaag aattttcaga tggacatgta |
| 7321 | caaatttgaa ctcaaaccat ccccagtcca gatacagggc agcgtgtagg tgaccacacc |
| 7381 | agagcctcag cctcggtcct tctcagccgt cgggatagga tccaggcatt tcttttaaat |
| 7441 | ctcagaggta gcagtaaact tttcagtatt gctgttagca agtgtgtgtt tgccaataga |
| 7501 | tacccattat actaatgtgc caagtaaatg ttcattgcac atctgcttcc actgtgttcc |
| 7561 | cacgggtgcc atgaagtgtg tgaggagccc ctcatctgga gggatgagtg ctgcgttgac |
| 7621 | tactgctatc aggattgtgt tgtgtggaat attcatctac ataaatttta tatgcacagt |
| 7681 | aatttccctt tttatatgtc aagtaactat ttgtaaaagt tatactcaca aattattata |
| 7741 | atgattacta atatattttt tccatgtttc attgcctgaa taaaaactgt ttaccactgt |
| 7801 | ta |

SEQ ID NO: 150 is the amino acid sequence of the FGFR1-TACC1 fusion protein.

MWSWKCLLFWAVLVTATLCTARPSPTLPEQDALPSSEDDDDDDDSSSEEK
ETDNTKPNPVAPYWTSPEKMEKKLHAVPAAKTVKFKCPSSGTPNPTLRWL
KNGKEFKPDHRIGGYKVRYATWSIIMDSVVPSDKGNYTCIVENEYGSINH
TYQLDVVERSPHRPILQAGLPANKTVALGSNVEFMCKVYSDPQPHIQWLK
HIEVNGSKIGPDNLPYVQILKTAGVNTTDKEMEVLHLRNVSFEDAGEYTC
LAGNSIGLSHHSAWLTVLEALEERPAVMTSPLYLEIIIYCTGAFLISCMV
GSVIVYKMKSGTKKSDFHSQMAVHKLAKSIPLRRQVTVSADSSASMNSGV
LLVRPSRLSSSGTPMLAGVSEYELPEDPRWELPRDRLVLGKPLGEGCFGQ
VVLAEAIGLDKDKPNRVTKVAVKMLKSDATEKDLSDLISEMEMMKMIGKH
KNIINLLGACTQDGPLYVIVEYASKGNLREYLQARRPPGLEYCYNPSHNP
EEQLSSKDLVSCAYQVARGMEYLASKKCIHRDLAARNVLVTEDNVMKIAD
FGLARDIHHIDYYKKTTNGRLPVKWMAPEALFDRIYTHQSDVWSFGVLLW
EIFTLGGSPYPGVPVEELFKLLKEGHRMDKPSNCTNELYMMMRDCWHAVP
SQRPTFKQLVEDLDRIVALTSNQGLLESSAEKAPVSVSCGGESPLDGICL
SESDKTAVLTLIREEIITKEIEANEWKKKYEETRQEVLEMRKIVAEYEKT
IAQMIEDEQRTSMTSQKSFQQLTMEKEQALADLNSVERSLSDLFRRYENL
KGVLEGFKKNEEALKKCAQDYLARVKQEEQRYQALKIHAEEKLDKANEEI
AQVRTKAKAESAALHAGLRKEQMKVESLERALQQKNQEIEELTKICDELI
AKLGKTD

SEQ ID NO: 151 is the nucleotide sequence that encodes the FGFR1-TACC1 fusion protein.

atgtggagctggaagtgcctcctcttctgggctgtgctggtcacagccac
actctgcaccgctaggccgtccccgaccttgcctgaacaagcccagccct
ggggagcccctgtggaagtggagtccttcctggtccaccccggtgacctg
ctgcagcttcgctgtcggctgcgggacgatgtgcagagcatcaactggct
gcgggacggggtgcagctggcggaaagcaaccgcacccgcatcacagggg
aggaggtggaggtgcaggactccgtgcccgcagactccggcctctatgct
tgcgtaaccagcagcccctcgggcagtgacaccacctacttctccgtcaa
tgtttcagatgctctcccctcctcggaggatgatgatgatgatgatgact
cctcttcagaggagaaagaaacagataacaccaaaccaaaccgtatgccc
gtagctccatattggacatccccagaaaagatggaaaagaaattgcatgc
agtgccggctgccaagacagtgaagttcaaatgcccttccagtgggaccc
caaaccccacactgcgctggttgaaaaatggcaaagaattcaaacctgac
cacagaattggaggctacaaggtccgttatgccacctggagcatcataat
ggactctgtggtgccctctgacaagggcaactacacctgcattgtggaga
atgagtacggcagcatcaaccacacataccagctggatgtcgtggagcgg
tcccctcaccggcccatcctgcaagcagggttgcccgccaacaaaacagt
ggccctgggtagcaacgtggagttcatgtgtaaggtgtacagtgacccgc
agccgcacatccagtggctaaagcacatcgaggtgaatgggagcaagatt
ggcccagacaacctgccttatgtccagatcttgaagactgctggagttaa
taccaccgacaaagagatggaggtgcttcacttaagaaatgtctcctttg
aggacgcaggggagtatacgtgcttggcgggtaactctatcggactctcc
catcactctgcatggttgaccgttctggaagccctggaagagaggccggc
agtgatgacctcgcccctgtacctggagatcatcatctattgcacagggg
ccttcctcatctcctgcatggtggggtcggtcatcgtctacaagatgaag
agtggtaccaagaagagtgacttccacagccagatggctgtgcacaagct
ggccaagagcatccctctgcgcagacaggtgtctgctgactccagtgcat
ccatgaactctggggttcttctggttcggccatcacggctctcctccagt
gggactcccatgctagcaggggtctctgagtatgagcttcccgaagaccc
tcgctgggagctgcctcgggacagactggtcttaggcaaacccctgggag
agggctgctttgggcaggtggtgttggcagaggctatcgggctggacaag
gacaaacccaaccgtgtgaccaaagtggctgtgaagatgttgaagtcgga
cgcaacagagaaagacttgtcagacctgatctcagaaatggagatgatga
agatgatcgggaagcataagaatatcatcaacctgctgggggcctgcacg
caggatggtcccttgtatgtcatcgtggagtatgcctccaagggcaacct
gcgggagtacctgcaggcccggaggcccccagggctggaatactgctaca
accccagccacaacccagaggagcagctctcctccaaggacctggtgtcc
tgcgcctaccaggtggcccgaggcatggagtatctggcctccaagaagtg
catacaccgagacctggcagccaggaatgtcctggtgacagaggacaatg
tgatgaagatagcagactttggcctcgcacgggacattcaccacatcgac
tactataaaaagacaaccaacggccgactgcctgtgaagtggatggcacc
cgaggcattatttgaccggatctacacccaccagagtgatgtgtggtctt
tcggggtgctcctgtgggagatcttcactctgggcggctccccatacccc
ggtgtgcctgtggaggaacttttcaagctgctgaaggagggtcaccgcat
ggacaagcccagtaactgcaccaacgagctgtacatgatgatgcgggact
gctggcatgcagtgccctcacagagacccaccttcaagcagctggtggaa
gacctggaccgcatcgtggccttgacctccaaccagtgggctgctggagt
cctctgcagagaaggcccctgtgtcggtgtcctgtggaggtgagagcccc
ctggatgggatctgcctcagcgaatcagacaagacagccgtgctcacctt
aataagagaagagataattactaaagagattgaagcaaatgaatggaaga
agaaatacgaagagacccggcaagaagttttggagatgaggaaaattgta
gctgaatatgaaaagactattgctcaaatgattgaagatgaacaaaggac
aagtatgacctctcagaagagcttccagcaactgaccatggagaaggaac
aggccctggctgaccttaactctgtggaaaggtccctttctgatctcttc
aggagatatgagaacctgaaaggtgttctggaagggttcaagaagaatga
agaagccttgaagaaatgtgctcaggattacttagccagagttaaacaag
aggagcagcgataccaggccctgaaaatccacgcagaagagaaactggac
aaagccaatgaagagattgctcaggttcgaacaaaagcaaaggctgagag
tgcagctctccatgctggactccgcaaagagcagatgaaggtggagtccc
tggaaagggccctgcagcagaagaaccaagaaattgaagaactgacaaaa
atctgtgatgagctgattgcaaagctgggaaagactgac

The Genbank ID for the FGFR2 gene is 2263. Eight isoforms are listed for FGFR2, e.g., having Genebank Accession Nos. NP_000132 (corresponding nucleotide sequence NM_000141); NP_001138385 (corresponding nucleotide sequence NM_001144913); NP_001138386 (corresponding nucleotide sequence NM_001144914); NP_001138387 (corresponding nucleotide sequence NM_001144915); NP_001138388 (corresponding nucleotide sequence NM_001144916); NP_001138389 (corresponding nucleotide sequence NM_001144917); NP_001138390 (corresponding nucleotide sequence NM_001144918); NP_001138391 (corresponding nucleotide sequence NM_001144919); NP_075259 (corresponding nucleotide sequence NM_022970).

SEQ ID NO: 152 is the FGFR2 Amino Acid Sequence for isoform 1, having Genebank Accession No. NP_000132 (821 aa):

| | |
|---|---|
| 1 | MVSWGRFICL VVVTMATLSL ARPSFSLVED TTLEPEEPPT KYQISQPEVY VAAPGESLEV |
| 61 | RCLLKDAAVI SWTKDGVHLG PNNRTVLIGE YLQIKGATPR DSGLYACTAS RTVDSETWYF |
| 121 | MVNVTDAISS GDDEDDTDGA EDFVSENSNN KRAPYWTNTE KMEKRLHAVP AANTVKFRCP |
| 181 | AGGNPMPTMR WLKNGKEFKQ EHRIGGYKVR NQHWSLIMES VVPSDKGNYT CVVENEYGSI |
| 241 | NHTYHLDVVE RSPHRPILQA GLPANASTVV GGDVEFVCKV YSDAQPHIQW IKHVEKNGSK |
| 301 | YGPDGLPYLK VLKAAGVNTT DKEIEVLYIR NVTFEDAGEY TCLAGNSIGI SFHSAWLTVL |
| 361 | PAPGREKEIT ASPDYLEIAI YCIGVFLIAC MVVTVILCRM KNTTKKPDFS SQPAVHKLTK |
| 421 | RIPLRRQVTV SAESSSSMNS NTPLVRITTR LSSTADTPML AGVSEYELPE DPKWEFPRDK |
| 481 | LTLGKPLGEG CFGQVVMAEA VGIDKDKPKE AVTVAVKMLK DDATEKDLSD LVSEMEMMKM |
| 541 | IGKHKNIINL LGACTQDGPL YVIVEYASKG NLREYLRARR PPGMEYSYDI NRVPEEQMTF |
| 601 | KDLVSCTYQL ARGMEYLASQ KCIHRDLAAR NVLVTENNVM KIADFGLARD INNIDYYKKT |
| 661 | TNGRLPVKWM APEALFDRVY THQSDVWSFG VLMWEIFTLG GSPYPGIPVE ELFKLLKEGH |
| 721 | RMDKPANCTN ELYMMMRDCW HAVPSQRPTF KQLVEDLDRI LTLTTNEEYL DLSQPLEQYS |
| 781 | PSYPDTRSSC SSGDDSVFSP DPMPYEPCLP QYPHINGSVK T |

SEQ ID NO: 153 is the FGFR2 Nucleotide Sequence for isoform 1, having Genebank Accession No. NM_000141 (4654 bp):

| | |
|---|---|
| 1 | ggcggcggct ggaggagagc gcggtggaga gccgagcggg cgggcggcgg gtgcggagcg |
| 61 | ggcgagggag cgcgcgcggc cgccacaaag ctcgggcgcc gcggggctgc atgcggcgta |
| 121 | cctggcccgg cgcggcgact gctctccggg ctggcggggg ccggccgcga gccccggggg |
| 181 | ccccgaggcc gcagcttgcc tgcgcgctct gagccttcgc aactcgcgag caaagtttgg |
| 241 | tggaggcaac gccaagcctg agtcctttct tcctctcgtt ccccaaatcc gagggcagcc |
| 301 | cgcgggcgtc atgcccgcgc tcctccgcag cctggggtac gcgtgaagcc cgggaggctt |
| 361 | ggcgccggcg aagacccaag gaccactctt ctgcgtttgg agttgctccc cgcaaccccg |
| 421 | ggctcgtcgc tttctccatc ccgacccacg cggggcgcgg ggacaacaca ggtcgcggag |
| 481 | gagcgttgcc attcaagtga ctgcagcagc agcggcagcg cctcggttcc tgagcccacc |
| 541 | gcaggctgaa ggcattgcgc gtagtccatg cccgtagagg aagtgtgcag atgggattaa |
| 601 | cgtccacatg gagatatgga agaggaccgg ggattggtac cgtaaccatg gtcagctggg |
| 661 | gtcgtttcat ctgcctggtc gtggtcacca tggcaacctt gtccctggcc cggccctcct |
| 721 | tcagtttagt tgaggatacc acattagagc cagaagagcc accaaccaaa taccaaatct |
| 781 | ctcaaccaga agtgtacgtg gctgcgccag gggagtcgct agaggtgcgc tgcctgttga |
| 841 | aagatgccgc cgtgatcagt tggactaagg atggggtgca cttggggccc aacaatagga |
| 901 | cagtgcttat tggggagtac ttgcagataa agggcgccac gcctagagac tccggcctct |
| 961 | atgcttgtac tgccagtagg actgtagaca gtgaaacttg gtacttcatg gtgaatgtca |
| 1021 | cagatgccat ctcatccgga gatgatgagg atgacaccga tggtgcggaa gattttgtca |
| 1081 | gtgagaacag taacaacaag agagcaccat actggaccaa cacagaaaag atggaaaagc |
| 1141 | ggctccatgc tgtgcctgcg gccaacactg tcaagtttcg ctgcccagcc ggggggaacc |
| 1201 | caatgccaac catgcggtgg ctgaaaaacg ggaaggagtt taagcaggag catcgcattg |
| 1261 | gaggctacaa ggtacgaaac cagcactgga gcctcattat ggaaagtgtg gtcccatctg |
| 1321 | acaagggaaa ttatacctgt gtagtggaga atgaatacgg gtccatcaat cacacgtacc |
| 1381 | acctggatgt tgtggagcga tcgcctcacc ggcccatcct ccaagccgga ctgccggcaa |
| 1441 | atgcctccac agtggtcgga ggagacgtag agtttgtctg caaggtttac agtgatgccc |
| 1501 | agccccacat ccagtggatc aagcacgtgg aaaagaacgg cagtaaatac gggcccgacg |
| 1561 | ggctgcccta cctcaaggtt ctcaaggccg ccggtgttaa caccacggac aaagagattg |
| 1621 | aggttctcta tattcggaat gtaacttttg aggacgctgg ggaatatacg tgcttggcgg |
| 1681 | gtaattctat tgggatatcc tttcactctg catggttgac agttctgcca gcgcctggaa |
| 1741 | gagaaaagga gattacagct tccccagact acctggagat agccatttac tgcatagggg |
| 1801 | tcttcttaat cgcctgtatg gtggtaacag tcatcctgtg ccgaatgaag aacacgacca |
| 1861 | agaagccaga cttcagcagc cagccggctg tgcacaagct gaccaaacgt atccccctgc |
| 1921 | ggagacaggt aacagtttcg gctgagtcca gctcctccat gaactccaac accccgctgg |
| 1981 | tgaggataac aacacgcctc tcttcaacgg cagacacccc catgctggca ggggtctccg |
| 2041 | agtatgaact tccagaggac ccaaaatggg agtttccaag agataagctg acactgggca |
| 2101 | agcccctggg agaaggttgc tttgggcaag tggtcatggc ggaagcagtg ggaattgaca |
| 2161 | aagacaagcc caaggaggcg gtcaccgtgg ccgtgaagat gttgaaagat gatgccacag |
| 2221 | agaaagacct ttctgatctg gtgtcagaga tggagatgat gaagatgatt gggaaacaca |
| 2281 | agaatatcat aaatcttctt ggagcctgca cacaggatgg gcctctctat gtcatagttg |
| 2341 | agtatgcctc taaaggcaac ctccgagaat acctccgagc ccggaggcca cccgggatgg |
| 2401 | agtactccta tgacattaac cgtgttcctg aggagcagat gaccttcaag gacttggtgt |
| 2461 | catgcaccta ccagctggcc agaggcatgg agtacttggc ttcccaaaaa tgtattcatc |
| 2521 | gagatttagc agccagaaat gttttggtaa cagaaaacaa tgtgatgaaa atagcagact |
| 2581 | ttggactcgc cagagatatc aacaatatag actattacaa aaagaccacc aatgggcggc |
| 2641 | ttccagtcaa gtggatggct ccagaagccc tgtttgatag agtatacact catcagagtg |
| 2701 | atgtctggtc cttcggggtg ttaatgtggg agatcttcac tttagggggc tcgccctacc |
| 2761 | cagggattcc cgtggaggaa ctttttaagc tgctgaagga aggacacaga atggataagc |
| 2821 | cagccaactg caccaacgaa ctgtacatga tgatgaggga ctgttggcat gcagtgccct |
| 2881 | cccagagacc aacgttcaag cagttggtag aagacttgga tcgaattctc actctcacaa |
| 2941 | ccaatgagga atacttggac ctcagccaac ctctcgaaca gtattcacct agttaccctg |
| 3001 | acacaagaag ttcttgttct tcaggagatg attctgtttt ttctccagac cccatgcctt |
| 3061 | acgaaccatg ccttcctcag tatccacaca taaacggcag tgttaaaaca tgaatgactg |
| 3121 | tgtctgcctg tccccaaaca ggacagcact gggaacctag ctacactgag cagggagacc |
| 3181 | atgcctccca gagcttgttg tctccacttg tatatatgga tcagaggagt aaataattgg |
| 3241 | aaaagtaatc agcatatgtg taaagattta tacagttgaa aacttgtaat cttccccagg |
| 3301 | aggagaagaa ggtttctgga gcagtggact gccacaagcc accatgtaac ccctctcacc |
| 3361 | tgccgtgcgt actggctgtg gaccagtagg actcaaggtg gacgtgcgtt ctgccttcct |
| 3421 | tgttaatttt gtaataattg gagaagattt atgtcagcac acacttacag agcacaaatg |
| 3481 | cagtatatag gtgctggatg tatgtaaata tattcaaatt atgtataaat atatattata |
| 3541 | tatttacaag gagttatttt ttgtattgat tttaaatgga tgtcccaatg cacctagaaa |
| 3601 | attggtctct ctttttttaa tagctatttg ctaaatgctg ttcttacaca taatttctta |
| 3661 | attttcaccg agcagaggtg gaaaaatact tttgctttca gggaaaatgg tataacgtta |
| 3721 | atttattaat aaattggtaa tatacaaaac aattaatcat ttatagtttt ttttgtaatt |
| 3781 | taagtggcat ttctatgcag gcagcacagc agactagtta atctattgct tggacttaac |
| 3841 | tagttatcag atcctttgaa aagagaatat ttacaatata tgactaattt ggggaaaatg |
| 3901 | aagttttgat ttatttgtgt ttaaatgctg ctgtcagacg attgttctta gacctcctaa |
| 3961 | atgccccata ttaaaagaac tcattcatag gaaggtgttt cattttggtg tgcaaccctg |
| 4021 | tcattacgtc aacgcaacgt ctaactggac ttcccaagat aaatggtacc agcgtcctct |
| 4081 | taaaagatgc cttaatccat tccttgagga cagaccttag ttgaaatgat agcagaatgt |
| 4141 | gcttctctct ggcagctggc cttctgcttc tgagttgcac attaatcaga ttagcctgta |
| 4201 | ttctcttcag tgaattttga taatggcttc cagactcttt ggcgttggag acgcctgtta |
| 4261 | ggatcttcaa gtcccatcat agaaaattga aacacagagt tgttctgctg atagttttgg |
| 4321 | ggatacgtcc atctttttaa gggattgctt tcatctaatt ctggcaggac ctcaccaaaa |
| 4381 | gatccagcct catacctaca tcagacaaaa tatcgccgtt gttccttctg tactaaagta |
| 4441 | ttgtgttttg ctttggaaac acccactcac tttgcaatag ccgtgcaaga tgaatgcaga |
| 4501 | ttacactgat cttatgtgtt acaaaattgg agaaagtatt taataaaacc tgttaatttt |
| 4561 | tatactgaca ataaaaatgt ttctacagat attaatgtta acaagacaaa ataaatgtca |
| 4621 | cgcaacttat ttttttaata aaaaaaaaaa aaaa |

The Genbank ID for the TACC2 gene is 10579. Four isoforms are listed for TACC2, e.g., having Genebank Accession Nos. NP_996744 (corresponding nucleotide sequence NM_206862); NP_996743 (corresponding nucleotide sequence NM_206861); NP_996742 (corresponding nucleotide sequence NM_206860); NP_008928 (corresponding nucleotide sequence NM_006997).

SEQ ID NO: 154 is the TACC2 Amino Acid Sequence for isoform a, having Genebank Accession No. NP_996744 (2948 aa):

| | |
|---|---|
| 1 | MGNENSTSDN QRTLSAQTPR SAQPPGNSQN IKRKQQDTPG SPDHRDASSI GSVGLGGFCT |
| 61 | ASESSASLDP CLVSPEVTEP RKDPQGARGP EGSLLPSPPP SQEREHPSSS MPFAECPPEG |
| 121 | CLASPAAAPE DGPQTQSPRR EPAPNAPGDI AAAFPAERDS STPYQEIAAV PSAGRERQPK |
| 181 | EEGQKSSFSF SSGIDQSPGM SPVPLREPMK APLCGEGDQP GGFESQEKEA AGGFPPAESR |
| 241 | QGVASVQVTP EAPAAAQQGT ESSAVLEKSP LKPMAPIPQD PAPRASDRER GQGEAPPQYL |
| 301 | TDDLEFLRAC HLPRSNSGAA PEAEVNAASQ ESCQQPVGAY LPHAELPWGL PSPALVPEAG |
| 361 | GSGKEALDTI DVQGHPQTGM RGTKPNQVVC VAAGGQPEGG LPVSPEPSLL TPTEEAHPAS |
| 421 | SLASFPAAQI PIAVEEPGSS SRESVSKAGM PVSADAAKEV VDAGLVGLER QVSDLGSKGE |
| 481 | HPEGDPGEVP APSPQERGEH LNTEQSHEVQ PGVPPPPLPK EQSHEVQPGA PPPPLPKAPS |
| 541 | ESARGPPGPT DGAKVHEDST SPAVAKEGSR SPGDSPGGKE EAPEPPDGGD PGNLQGEDSQ |
| 601 | AFSSKRDPEV GKDELSKPSS DAESRDHPSS HSAQPPRKGG AGHTDGPHSQ TAEADASGLP |
| 661 | HKLGEEDPVL PPVPDGAGEP TVPEGAIWEG SGLQPKCPDT LQSREGLGRM ESFLTLESEK |
| 721 | SDFPPTPVAE VAPKAQEGES TLEIRKMGSC DGEGLLTSPD QPRGPACDAS RQEFHAGVPH |
| 781 | PPQGENLAAD LGLTALILDQ DQQGIPSCPG EGWIRGAASE WPLLSSEKHL QPSQAQPETS |
| 841 | IFDVLKEQAQ PPENGKETSP SHPGFKDQGA DSSQIHVPVE PQEDNNLPTH GGQEQALGSE |
| 901 | LQSQLPKGTL SDTPTSSPTD MVWESSLTEE SELSAPTRQK LPALGEKRPE GACGDGQSSR |
| 961 | VSPPAADVLK DFSLAGNFSR KETCCTGQGP NKSQQALADA LEEGSQHEEA CQRHPGASEA |
| 1021 | ADGCSPLWGL SKREMASGNT GEAPPCQPDS VALLDAVPCL PALAPASPGV TPTQDAPETE |
| 1081 | ACDETQEGRQ QPVPAPQQKM ECWATSDAES PKLLASFPSA GEQGGEAGAA ETGGSAGAGD |
| 1141 | PGKQQAPEKP GEATLSCGLL QTEHCLTSGE EASTSALRES CQAEHPMASC QDALLPAREL |
| 1201 | GGIPRSTMDF STHQAVPDPK ELLLSGPPEV AAPDTPYLHV DSAAQRGAED SGVKAVSSAD |
| 1261 | PRAPGESPCP VGEPPLALEN AASLKLFAGS LAPLLQPGAA GGEIPAVQAS SGSPKARTTE |
| 1321 | GPVDSMPCLD RMPLLAKGKQ ATGEEKAATA PGAGAKASGE GMAGDAAGET EGSMERMGEP |
| 1381 | SQDPKQGTSG GVDTSSEQIA TLTGFPDFRE HIAKIFEKPV LGALATPGEK AGAGRSAVGK |
| 1441 | DLTRPLGPEK LLDGPPGVDV TLLPAPPARL QVEKKQQLAG EAEISHLALQ DPASDKLLGP |
| 1501 | AGLTWERNLP GAGVGKEMAG VPPTLREDER PEGPGAAWPG LEGQAYSQLE RSRQELASGL |
| 1561 | PSPAATQELP VERAAAFQVA PHSHGEEAVA QDRIPSGKQH QETSACDSPH GEDGPGDFAH |
| 1621 | TGVPGHVPRS TCAPSPQREV LTVPEANSEP WTLDTLGGER RPGVTAGILE MRNALGNQST |
| 1681 | PAPPTGEVAD TPLEPGKVAG AAGEAEGDIT LSTAETQACA SGDLPEAGTT RTFSVVAGDL |
| 1741 | VLPGSCQDPA CSDKAPGMEG TAALHGDSPA RPQQAKEQPG PERPIPAGDG KVCVSSPPEP |
| 1801 | DETHDPKLQH LAPEELHTDR ESPRPGPSML PSVPKKDAPR VMDKVTSDET RGAEGTESSP |
| 1861 | VADDIIQPAA PADLESPTLA ASSYHGDVVG QVSTDLIAQS ISPAAAHAGL PPSAAEHIVS |
| 1921 | PSAPAGDRVE ASTPSCPDPA KDLSRSSDSE EAFETPESTT PVKAPPAPPP PPPEVIPEPE |
| 1981 | VSTQPPPEEP GCGSETVPVP DGPRSDSVEG SPFRPPSHSF SAVFDEDKPI ASSGTYNLDF |
| 2041 | DNIELVDTFQ TLEPRASDAK NQEGKVNTRR KSTDSVPISK STLSRSLSLQ ASDFDGASSS |
| 2101 | GNPEAVALAP DAYSTGSSSA SSTLKRTKKP RPPSLKKKQT TKKPTETPPV KETQQEPDEE |
| 2161 | SLVPSGENLA SETKTESAKT EGPSPALLEE TPLEPAVGPK AACPLDSESA EGVVPPASGG |
| 2221 | GRVQNSPPVG RKTLPLTTAP EAGEVTPSDS GGQEDSPAKG LSVRLEFDYS EDKSSWDNQQ |
| 2281 | ENPPPTKKIG KKPVAKMPLR RPKMKKTPEK LDNTPASPPR SPAEPNDIPI AKGTYTFDID |
| 2341 | KWDDPNFNPF SSTSKMQESP KLPQQSYNFD PDTCDESVDP FKTSSKTPSS PSKSPASFEI |
| 2401 | PASAMEANGV DGDGLNKPAK KKKTPLKTDT FRVKKSPKRS PLSDPPSQDP TPAATPETPP |
| 2461 | VISAVVHATD EEKLAVTNQK WTCMTVDLEA DKQDYPQPSD LSTFVNETKF SSPTEELDYR |
| 2521 | NSYEIEYMEK IGSSLPQDDD APKKQALYLM FDTSQESPVK SSPVRMSESP TPCSGSSFEE |
| 2581 | TEALVNTAAK NQHPVPRGLA PNQESHLQVP EKSSQKELEA MGLGTPSEAI EITAPEGSFA |
| 2641 | SADALLSRLA HPVSLCGALD YLEPDLAEKN PPLFAQKLQE ELEFAIMRIE ALKLARQIAL |
| 2701 | ASRSHQDAKR EAAHPTDVSI SKTALYSRIG TAEVEKPAGL LFQQPDLDSA LQIARAEIIT |
| 2761 | KEREVSEWKD KYEESRREVM EMRKIVAEYE KTIAQMIEDE QREKSVSHQT VQQLVLEKEQ |
| 2821 | ALADLNSVEK SLADLFRRYE KMKEVLEGFR KNEEVLKRCA QEYLSRVKKE EQRYQALKVH |
| 2881 | AEEKLDRANA EIAQVRGKAQ QEQAAHQASL RKEQLRVDAL ERTLEQKNKE IEELTKICDE |
| 2941 | LIAKMGKS |

SEQ ID NO: 155 is the TACC2 Nucleotide Sequence for isoform a, having Genebank Accession No. NM_206862 (9706 bp)

| | |
|---|---|
| 1 | gcctgctcca agggaaggat caggagagaa gaaacgcaaa tcccagaacc gtgccaacat |
| 61 | ataaaacccc acattaaggg ttgtacagtg cactgggatt tctcaagtca cccgcttggt |
| 121 | cctcttccaa gtatacttta cttcctttca ttcctctcta aaactttttt aaaaactttc |
| 181 | actcctgctc taaaagttat cttggtttct tactctacct tatgcccctt gggcgaattt |
| 241 | tttcctctga ggagggaaga atagagttgc tgctgcagac acatcagatt ccctactggt |
| 301 | aacagctgga gtgcgtcacc tctgacaaaa ttctggggac gctgggaaca ctgaatcaac |
| 361 | atgggcaatg agaacagcac ctcggacaac cagaggactt tatcagctca gactccaagg |
| 421 | tccgcgcagc cacccgggaa cagtcagaat ataaaaagga agcagcagga cacgcccgga |
| 481 | agccctgacc acagagacgc gtccagcatt ggcagcgttg ggcttggagg cttctgcacc |
| 541 | gcttctgaga gttctgccag cctggatcca tgccttgtgt ccccagaggt gactgagcca |
| 601 | aggaaggacc cacagggagc cagggggcca gaaggttctt tgctgcccag cccaccaccg |
| 661 | tcccaggagc gagagcaccc ctcgtcctcc atgccctttg ccgagtgtcc cccggaaggt |
| 721 | tgcttggcaa gtccagcagc ggcacctgaa gatggtcctc agactcagtc tcccaggagg |
| 781 | gaacctgccc caaatgcccc aggagacatc gcggcggcat ttcccgctga gagggacagc |
| 841 | tctactccat accaagagat tgctgccgtc cccagtgctg gaagagagag acagccgaag |
| 901 | gaagaaggac agaagtcctc cttctccttc tccagtggca tcgaccagtc acctggaatg |
| 961 | tcgccagtac ccctcagaga gccaatgaag gcaccgctgt gtggagaggg ggaccagcct |
| 1021 | ggtggttttg agtcccaaga gaaagaggct gcaggtggct ttccccctgc agagtccagg |
| 1081 | cagggggtgg cttctgtgca agtgacccct gaggcccctg ctgcagccca gcagggcaca |
| 1141 | gaaagctcag cggtcttgga gaagtccccc ctaaaaccca tggccccgat cccacaagat |
| 1201 | ccagccccaa gagcctcaga cagagaaaga ggccaagggg aggcgccgcc tcagtattta |
| 1261 | acagatgact tggaattcct cagggcctgc catctcccta ggagcaattc aggggctgcc |
| 1321 | ccagaagcag aagtgaatgc cgcttcccag gagagctgcc agcagccagt gggagcatat |
| 1381 | ctgccgcacg cagagctgcc ctggggcttg ccaagtcctg ccctggtgcc agaggctggg |
| 1441 | ggctctggga aggaggctct ggacaccatt gatgttcagg gtcacccaca gacagggatg |
| 1501 | cgaggaacca agcccaatca agttgtctgt gtggcagcag gcggccagcc cgaagggggt |
| 1561 | ttgcctgtga gccctgaacc ttccctgctc actccgactg aggaagcaca tccagcttca |
| 1621 | agcctcgctt cattcccagc tgctcagatt cctattgctg tagaagaacc tggatcatca |
| 1681 | tccagggaat cagtttccaa ggctgggatg ccagtttctg cagatgcagc caaagaggtg |
| 1741 | gtggatgcag ggttggtggg actggagagg caggtgtcag atcttggaag caagggagag |
| 1801 | catccagaag gggaccctgg agaggttcct gccccatcac cccaggagag gggagagcac |
| 1861 | ttgaacacgg agcaaagcca tgaggtccaa ccaggagtac caccccctcc tcttcccaag |
| 1921 | gagcaaagcc atgaggtcca accaggagca ccaccccctc ctcttcccaa ggcaccaagt |
| 1981 | gaaagtgcca gagggccacc ggggccaacg gatggagcca aggtccatga agattccaca |
| 2041 | agcccagccg tggctaaaga aggaagcaga tcacctggtg acagccctgg aggaaaggag |
| 2101 | gaagccccag agccacctga tggtggagac ccagggaacc tgcaaggaga ggactctcag |
| 2161 | gctttcagca gcaagcgtga tccagaagta ggcaaagatg agctttcaaa gccaagcagt |
| 2221 | gatgcagaga gcagagacca tcccagctca cactcagcac agccacccag aaaggggggt |
| 2281 | gctgggcaca cggacgggcc ccactctcag acagcagagg ctgatgcatc tggcctacca |
| 2341 | cacaagctgg gtgaggagga ccccgtcctg ccccctgtgc cagatggagc tggtgagccc |
| 2401 | actgttcccg aaggagccat ctgggagggg tcaggattgc agcccaaatg tcctgacacc |
| 2461 | cttcagagca gggaaggatt gggaagaatg gagtctttcc tgactttaga atcagagaaa |
| 2521 | tcagattttc caccaactcc tgttgcagag gttgcaccca aagcccagga aggtgagagc |
| 2581 | acattggaaa taaggaagat gggcagctgt gatggggagg gcttgctgac gtccccagat |
| 2641 | caaccccgcg ggccggcgtg tgatgcgtcg agacaggaat ttcatgctgg ggtgccacat |
| 2701 | cccccccagg gggagaactt ggcagcagac ctggggctca cggcactcat cctggaccaa |
| 2761 | gatcagcagg gaatcccatc ctgcccaggg gaaggctgga taagaggagc tgcatccgag |
| 2821 | tggcccctac tatcttctga gaagcatctc cagccatccc aggcacaacc agagacatcc |
| 2881 | atctttgacg tgctcaagga gcaggcccag ccacctgaaa atgggaaaga gacttctcca |
| 2941 | agccatccag gttttaagga ccagggagca gattcttccc aaatccatgt acctgtggaa |
| 3001 | cctcaggaag ataacaactt gcccactcat ggaggacagg agcaggcttt gggatcagaa |
| 3061 | cttcaaagtc agctccccaa aggcaccctg tctgatactc caacttcatc tcccactgac |
| 3121 | atggtttggg agagttctct gacagaagag tcagaattgt cagcaccaac gagacagaag |
| 3181 | ttgcctgcac taggggagaa gcggccagag ggagcatgcg gtgatggtca gtcctcgagg |
| 3241 | gtctcgcctc cagcagcaga tgtcttaaaa gacttttctc ttgcagggaa cttcagcaga |
| 3301 | aaggaaactt gctgcactgg gcaggggcca aacaagtctc aacaggcatt ggctgatgcc |
| 3361 | ttggaagaag gcagccagca tgaagaagca tgtcaaaggc atccaggagc ttctgaagca |
| 3421 | gctgatggtt gttccccact ctggggcttg agtaagaggg agatggcaag tggaaacaca |
| 3481 | ggggaggccc caccttgtca gcctgactca gtagctctcc tggatgcagt tccctgcctg |
| 3541 | ccagccctgg cgcccgccag ccccggagtc acacccaccc aggatgcccc agagacagag |
| 3601 | gcatgtgatg aaacccagga aggcaggcag caaccagtgc cggccccgca gcagaaaatg |
| 3661 | gagtgctggg ccacttcgga tgcagagtcc ccaaagcttc ttgcaagttt cccatcagct |
| 3721 | ggggagcaag gtggtgaagc cggggctgct gagactggtg gcagcgctgg tgcaggagac |
| 3781 | ccaggaaagc agcaggctcc ggagaaacct ggagaagcta ctttgagttg tggcctcctt |
| 3841 | cagactgagc actgccttac ctccggggag gaagcttcta cctctgccct acgtgagtcc |
| 3901 | tgccaagctg agcaccccat ggccagctgc caggatgcct tgctgccagc cagagagctg |
| 3961 | ggtgggattc ccaggagcac catggatttt tctacacacc aggctgtccc agacccaaag |
| 4021 | gagctcctgc tgtctgggcc accagaagtg gctgctcctg acacccctta cctgcatgtc |
| 4081 | gacagtgctg cccagagagg agcagaagac agtggagtga aagctgtttc ctctgcagac |
| 4141 | cccagagctc ctggcgaaag cccctgtcct gtaggggagc ccccacttgc cttggaaaat |
| 4201 | gctgcctcct tgaagctgtt tgctggctcc ctcgcccccc tgttgcaacc aggagctgca |
| 4261 | ggtggggaaa tccctgcagt gcaagccagc agtggtagtc ccaaagccag aaccactgag |
| 4321 | ggaccagtgg actccatgcc atgcctggac cggatgccac ttctggccaa gggcaagcag |
| 4381 | gcaacagggg aagagaaagc agcaacagct ccaggtgcag gtgccaaggc cagtggggag |
| 4441 | ggcatggcag gtgatgcagc aggagagaca gagggcagca tggagaggat gggagagcct |
| 4501 | tcccaggacc caaagcaggg cacatcaggt ggtgtggaca caagctctga gcaaatcgcc |
| 4561 | accctcactg gcttcccaga cttcagggag cacatcgcca agatcttcga gaagcctgtg |
| 4621 | ctcggagccc tggccacacc tggagaaaag gcaggagctg ggaggagtgc agtgggtaaa |
| 4681 | gacctcacca ggccattggg cccagagaag cttctagatg ggcctccagg agtggatgtc |
| 4741 | acccttctcc ctgcacctcc tgctcgactc caggtggaga agaagcaaca gttggctgga |
| 4801 | gaggctgaga tttcccatct ggctctgcaa gatccagctt cagacaagct tctgggtcca |
| 4861 | gcagggctga cctgggagcg gaacttgcca ggtgccggtg tggggaagga gatggcaggt |
| 4921 | gtcccaccca cactgaggga agacgagagg ccagaggggc ctggggcagc ctggccaggc |
| 4981 | ctggaaggcc aggcttactc acagctggag aggagcaggc aggaattagc ttcaggtctt |
| 5041 | ccttcaccag cagctactca ggagctccct gtggagagag ctgctgcctt ccaggtggct |
| 5101 | ccccatagcc atggagaaga ggccgtggcc caagacagaa ttccttctgg aaagcagcac |
| 5161 | caggaaacat ctgcctgcga cagtccacat ggagaagatg gtcccgggga ctttgctcac |
| 5221 | acaggggttc caggacatgt gccaaggtcc acgtgtgccc cttctcctca gagggaggtt |
| 5281 | ttgactgtgc ctgaggccaa cagtgagccc tggacccttg acacgcttgg gggtgaaagg |
| 5341 | agacccggag tcactgctgg catcttggaa atgcgaaatg ccctgggcaa ccagagcacc |
| 5401 | cctgcaccac caactggaga agtggcagac actcccctgg agcctggcaa ggtggcaggc |
| 5461 | gctgctgggg aagcagaggg tgacatcacc ctgagcacag ctgagacaca ggcatgtgcg |
| 5521 | tccggtgatc tgcctgaagc aggtactacg aggacattct ccgttgtggc aggtgacttg |
| 5581 | gtgctgccag gaagctgtca ggacccagcc tgctctgaca aggctccggg gatggagggt |
| 5641 | acagctgccc ttcatgggga cagcccagcc aggccccagc aggctaagga gcagccaggg |
| 5701 | cctgagcgcc ccattccagc tggggatggg aaggtgtgcg tctcctcacc tccagagcct |
| 5761 | gacgaaactc acgacccgaa gctgcaacat ttggctccag aagagctcca cactgacaga |
| 5821 | gagagcccca ggcctggccc atccatgtta ccttcggttc ctaagaagga tgctccaaga |
| 5881 | gtcatggata aagtcacttc agatgagacc agaggtgcgg aaggaacaga aagttcacct |
| 5941 | gtggcagatg atatcatcca gcccgctgcc cccgcagacc tggaaagccc aaccttagct |
| 6001 | gcctcttcct accacggtga tgttgttggc caggtctcta cggatctgat agcccagagc |
| 6061 | atctccccag ctgctgccca tgcgggtctt cctccctcgg ctgcagaaca catagtttcg |
| 6121 | ccatctgccc cagctggtga cagagtagaa gcttccactc cctcctgccc agatccggcc |
| 6181 | aaggacctca gcaggagttc cgattctgaa gaggcatttg agaccccgga gtcaacgacc |
| 6241 | cctgtcaaag ctccgccagc tccaccccca ccaccccccg aagtcatccc agaacccgag |
| 6301 | gtcagcacac agccaccccc ggaagaacca ggatgtggtt ctgagacagt ccctgtccct |
| 6361 | gatggcccac ggagcgactc ggtggaagga agtcccttcc gtcccccgtc acactccttc |
| 6421 | tctgccgtct tcgatgaaga caagccgata gccagcagtg ggacttacaa cttggacttt |
| 6481 | gacaacattg agcttgtgga tacctttcag accttggagc ctcgtgcctc agacgctaag |
| 6541 | aatcaggagg gcaaagtgaa cacacggagg aagtccacgg attccgtccc catctctaag |
| 6601 | tctacactgt cccggtcgct cagcctgcaa gccagtgact ttgatggtgc ttcttcctca |
| 6661 | ggcaatcccg aggccgtggc ccttgcccca gatgcatata gcacgggttc cagcagtgct |
| 6721 | tctagtaccc ttaagcgaac taaaaaaccg aggccgcctt ccttaaaaaa gaaacagacc |
| 6781 | accaagaaac ccacagagac ccccccagtg aaggagacgc aacaggagcc agatgaagag |
| 6841 | agccttgtcc ccagtgggga gaatctagca tctgagacga aaacggaatc tgccaagacg |
| 6901 | gaaggtccta gcccagcctt attggaggag acgccccttg agcccgctgt ggggcccaaa |
| 6961 | gctgcctgcc ctctggactc agagagtgca gaaggggttg tccccccggc ttctggaggt |
| 7021 | ggcagagtgc agaactcacc ccctgtcggg aggaaaacgc tgcctcttac cacggccccg |
| 7081 | gaggcagggg aggtaacccc atcggatagc ggggggcaag aggactctcc agccaaaggg |
| 7141 | ctctccgtaa ggctggagtt tgactattct gaggacaaga gtagttggga caaccagcag |
| 7201 | gaaaaccccc ctcctaccaa aaagataggc aaaaagccag ttgccaaaat gcccctgagg |
| 7261 | aggccaaaga tgaaaaagac acccgagaaa cttgacaaca ctcctgcctc acctcccaga |
| 7321 | tcccctgctg aacccaatga catccccatt gctaaaggta cttacacctt tgatattgac |
| 7381 | aagtgggatg accccaattt taaccctttt tcttccacct caaaaatgca ggagtctccc |
| 7441 | aaactgcccc aacaatcata caactttgac ccagacacct gtgatgagtc cgttgacccc |
| 7501 | tttaagacat cctctaagac ccccagctca ccttctaaat ccccagcctc ctttgagatc |
| 7561 | ccagccagtg ctatggaagc caatggagtg gacggggatg ggctaaacaa gcccgccaag |
| 7621 | aagaagaaga cgcccctaaa gactgacaca tttagggtga aaaagtcgcc aaaacggtct |
| 7681 | cctctctctg atccaccttc ccaggacccc accccagctg ctacaccaga aacaccacca |
| 7741 | gtgatctctg cggtggtcca cgccacagat gaggaaaagc tggcggtcac caaccagaag |
| 7801 | tggacgtgca tgacagtgga cctagaggct gacaaacagg actacccgca gccctcggac |
| 7861 | ctgtccacct ttgtaaacga gaccaaattc agttcaccca ctgaggagtt ggattacaga |
| 7921 | aactcctatg aaattgaata tatggagaaa attggctcct ccttacctca ggacgacgat |
| 7981 | gccccgaaga agcaggcctt gtaccttatg tttgacactt ctcaggagag ccctgtcaag |
| 8041 | tcatctcccg tccgcatgtc agagtccccg acgccgtgtt cagggtcaag ttttgaagag |
| 8101 | actgaagccc ttgtgaacac tgctgcgaaa aaccagcatc ctgtcccacg aggactggcc |
| 8161 | cctaaccaag agtcacactt gcaggtgcca gagaaatcct cccagaagga gctggaggcc |
| 8221 | atgggcttgg gcaccccttc agaagcgatt gaaattacag ctcccgaggg ctcctttgcc |
| 8281 | tctgctgacg ccctcctcag caggctagct caccccgtct ctctctgtgg tgcacttgac |
| 8341 | tatctggagc ccgacttagc agaaaagaac cccccactat tcgctcagaa actccaggag |
| 8401 | gagttagagt ttgccatcat gcggatagaa gccctgaagc tggccaggca gattgctttg |
| 8461 | gcttcccgca gccaccagga tgccaagaga gaggctgctc acccaacaga cgtctccatc |
| 8521 | tccaaaacag ccttgtactc ccgcatcggg accgctgagg tggagaaacc tgcaggcctt |
| 8581 | ctgttccagc agcccgacct ggactctgcc ctccagatcg ccagagcaga gatcataacc |
| 8641 | aaggagagag aggtctcaga atggaaagat aaatatgaag aaagcaggcg ggaagtgatg |
| 8701 | gaaatgagga aaatagtggc cgagtatgag aagaccatcg ctcagatgat agaggacgaa |
| 8761 | cagagagaga agtcagtctc ccaccagacg gtgcagcagc tggttctgga gaaggagcaa |
| 8821 | gccctggccg acctgaactc cgtggagaag tctctggccg acctcttcag aagatatgag |
| 8881 | aagatgaagg aggtcctaga aggcttccgc aagaatgaag aggtgttgaa gagatgtgcg |
| 8941 | caggagtacc tgtcccgggt gaagaaggag gagcagaggt accaggccct gaaggtgcac |
| 9001 | gcggaggaga aactggacag ggccaatgct gagattgctc aggttcgagg caaggcccag |
| 9061 | caggagcaag ccgcccacca ggccagcctg cggaaggagc agctgcgagt ggacgccctg |
| 9121 | gaaaggacgc tggagcagaa gaataaagaa atagaagaac tcaccaagat ttgtgacgaa |
| 9181 | ctgattgcca aaatggggaa aagctaactc tgaaccgaat gttttggact taactgttgc |
| 9241 | gtgcaatatg accgtcggca cactgctgtt cctccagttc catggacagg ttctgttttc |
| 9301 | actttttcgt atgcactact gtatttcctt tctaaataaa attgatttga ttgtatgcag |
| 9361 | tactaaggag actatcagaa tttcttgcta ttggtttgca ttttcctagt ataattcata |
| 9421 | gcaagttgac ctcagagttc ctgtatcagg gagattgtct gattctctaa taaaagacac |
| 9481 | attgctgacc ttggccttgc cctttgtaca caagttccca gggtgagcag cttttggatt |
| 9541 | taatatgaac atgtacagcg tgcataggga ctcttgcctt aaggagtgta aacttgatct |
| 9601 | gcatttgctg atttgttttt aaaaaaacaa gaaatgcatg tttcaaataa aattctctat |
| 9661 | tgtaaataaa attttttctt tggatcttgg caaaaaaaaa aaaaaa |

SEQ ID NO: 158 is the amino acid sequence of the FGFR3-TACC3-1 fusion protein. The bolded text corresponds to the FGFR3 protein:

MGAPACALALCVAVAIVAGASSESLGTEQRVVGRAAEVPGPEPGQQEQLV
FGSGDAVELSCPPPGGGPMGPTVWVKDGTGLVPSERVLVGPQRLQVLNAS
HEDSGAYSCRQRLTQRVLCHFSVRVTDAPSSGDDEDGEDEAEDTGVDTGA
PYWTRPERMDKKLLAVPAANTVRFRCPAAGNPTPSISWLKNGREFRGEHR
IGGIKLRHQQWSLVMESVVPSDRGNYTCVVENKFGSIRQTYTLDVLERSP
HRPILQAGLPANQTAVLGSDVEFHCKVYSDAQPHIQWLKHVEVNGSKVGP
DGTPYVTVLKTAGANTTDKELEVLSLHNVTFEDAGEYTCLAGNSIGFSHH
SAWLVVLPAEEELVEADEAGSVYAGILSYGVGFFLFILVVAAVTLCRLRS
PPKKGLGSPTVHKISRFPLKRQVSLESNASMSSNTPLVRIARLSSGEGPT
LANVSELELPADPKWELSRARLTLGKPLGEGCFGQVVMAEAIGIDKDRAA
KPVTVAVKMLKDDATDKDLSDLVSEMEMMKMIGKHKNIINLLGACTQGGP
LYVLVEYAAKGNLREFLRARRPPGLDYSFDTCKPPEEQLTFKDLVSCAYQ
VARGMEYLASQKCIHRDLAARNVLVTEDNVMKIADFGLARDVHNLDYYKK
TTNGRLPVKWMAPEALFDRVYTHQSDVWSFGVLLWEIFTLGGSPYPGIPV
EELFKLLKEGHRMDKPANCTHDLYMIMRECWHAAPSQRPTFKQLVEDLDR
VLTVTSTDFKESALRKQSLYLKFDPLLRDSPGRPVPVATETSSMHGANET
PSGRPREAKLVEFDFLGALDIPVPGPPPGVPAPGGPPLSTGPIVDLLQYS
QKDLDAVVKATQEENRELRSRCEELHGKNLELGKIMDRFEEVVYQAMEEV
QKQKELSKAEIQKVLKEKDQLTTDLNSMEKSFSDLFKRFEKQKEVIEGYR
KNEESLKKCVEDYLARITQEGQRYQALKAHAEEKLQLANEEIAQVRSKAQ
AEALALQASLRKEQMRIQSLEKTVEQKTKENEELTRICDDLISKMEKI

SEQ ID NO: 159 is the amino acid sequence of the FGFR3-TACC3-2 fusion protein. The bolded text corresponds to the FGFR3 protein:

MGAPACALALCVAVAIVAGASSESLGTEQRVVGRAAEVPGPEPGQQEQLV
FGSGDAVELSCPPPGGGPMGPTVWVKDGTGLVPSERVLVGPQRLQVLNAS
HEDSGAYSCRQRLTQRVLCHFSVRVTDAPSSGDDEDGEDEAEDTGVDTGA
PYWTRPERMDKKLLAVPAANTVRFRCPAAGNPTPSISWLKNGREFRGEHR
IGGIKLRHQQWSLVMESVVPSDRGNYTCVVENKFGSIRQTYTLDVLERSP
HRPILQAGLPANQTAVLGSDVEFHCKVYSDAQPHIQWLKHVEVNGSKVGP
DGTPYVTVLKTAGANTTDKELEVLSLHNVTFEDAGEYTCLAGNSIGFSHH
SAWLVVLPAEEELVEADEAGSVYAGILSYGVGFFLFILVVAAVTLCRLRS
PPKKGLGSPTVHKISRFPLKRQVSLESNASMSSNTPLVRIARLSSGEGPT
LANVSELELPADPKWELSRARLTLGKPLGEGCFGQVVMAEAIGIDKDRAA
KPVTVAVKMLKDDATDKDLSDLVSEMEMMKMIGKHKNIINLLGACTQGGP
LYVLVEYAAKGNLREFLRARRPPGLDYSFDTCKPPEEQLTFKDLVSCAYQ
VARGMEYLASQKCIHRDLAARNVLVTEDNVMKIADFGLARDVHNLDYYKK
TTNGRLPVKWMAPEALFDRVYTHQSDVWSFGVLLWEIFTLGGSPYPGIPV
EELFKLLKEGHRMDKPANCTHDLYMIMRECWHAAPSQRPTFKQLVEDLDR
VLTVTSTDVSAGSGLVPPAYAPPPAVPGHPSGRPREAKLVEFDFLGALDI
PVPGPPPGVPAPGGPPLSTGPIVDLLQYSQKDLDAVVKATQEENRELRSR
CEELHGKNLELGKIMDRFEEVVYQAMEEVQKQKELSKAEIQKVLKEKDQL
TTDLNSMEKSFSDLFKRFEKQKEVIEGYRKNEESLKKCVEDYLARITQEG
QRYQALKAHAEEKLQLANEEIAQVRSKAQAEALALQASLRKEQMRIQSLE
KTVEQKTKENEELTRICDDLISKMEKI

SEQ ID NO: 160 is the amino acid sequence of the FGFR3-TACC3-3 fusion protein. The bolded text corresponds to the FGFR3 protein:

MGAPACALALCVAVAIVAGASSESLGTEQRVVGRAAEVPGPEPGQQEQLV
FGSGDAVELSCPPPGGGPMGPTVWVKDGTGLVPSERVLVGPQRLQVLNAS
HEDSGAYSCRQRLTQRVLCHFSVRVTDAPSSGDDEDGEDEAEDTGVDTGA
PYWTRPERMDKKLLAVPAANTVRFRCPAAGNPTPSISWLKNGREFRGEHR
IGGIKLRHQQWSLVMESVVPSDRGNYTCVVENKFGSIRQTYTLDVLERSP
HRPILQAGLPANQTAVLGSDVEFHCKVYSDAQPHIQWLKHVEVNGSKVGP
DGTPYVTVLKTAGANTTDKELEVLSLHNVTFEDAGEYTCLAGNSIGFSHH
SAWLVVLPAEEELVEADEAGSVYAGILSYGVGFFLFILVVAAVTLCRLRS
PPKKGLGSPTVHKISRFPLKRQVSLESNASMSSNTPLVRIARLSSGEGPT
LANVSELELPADPKWELSRARLTLGKPLGEGCFGQVVMAEAIGIDKDRAA
KPVTVAVKMLKDDATDKDLSDLVSEMEMMKMIGKHKNIINLLGACTQGGP
LYVLVEYAAKGNLREFLRARRPPGLDYSFDTCKPPEEQLTFKDLVSCAYQ
VARGMEYLASQKCIHRDLAARNVLVTEDNVMKIADFGLARDVHNLDYYKK
TTNGRLPVKWMAPEALFDRVYTHQSDVWSFGVLLWEIFTLGGSPYPGIPV
EELFKLLKEGHRMDKPANCTHDLYMIMRECWHAAPSQRPTFKQLVEDLDR
VLTVTSTDVPGPPPGVPAPGGPPLSTGPIVDLLQYSQKDLDAVVKATQEE
NRELRSRCEELHGKNLELGKIMDRFEEVVYQAMEEVQKQKELSKAEIQKV
KEKDQLTTDLNSMEKSFSDLFKRFEKQKEVIEGYRKNEESLKKCVEDYL
ARITQEGQRYQALKAHAEEKLQLANEEIAQVRSKAQAEALALQASLRKEQ
MRIQSLEKTVEQKTKENEELTRICDDLISKMEKI

SEQ ID NO: 161 is the amino acid sequence of the FGFR3-TACC3-4 fusion protein. The bolded text corresponds to the FGFR3 protein:

MGAPACALALCVAVAIVAGASSESLGTEQRVVGRAAEVPGPEPGQQEQLV
FGSGDAVELSCPPPGGGPMGPTVWVKDGTGLVPSERVLVGPQRLQVLNAS
HEDSGAYSCRQRLTQRVLCHFSVRVTDAPSSGDDEDGEDEAEDTGVDTGA
PYWTRPERMDKKLLAVPAANTVRFRCPAAGNPTPSISWLKNGREFRGEHR
IGGIKLRHQQWSLVMESVVPSDRGNYTCVVENKFGSIRQTYTLDVLERSP
HRPILQAGLPANQTAVLGSDVEFHCKVYSDAQPHIQWLKHVEVNGSKVGP
DGTPYVTVLKTAGANTTDKELEVLSLHNVTFEDAGEYTCLAGNSIGFSHH
SAWLVVLPAEEELVEADEAGSVYAGILSYGVGFFLFILVVAAVTLCRLRS
PPKKGLGSPTVHKISRFPLKRQVSLESNASMSSNTPLVRIARLSSGEGPT
LANVSELELPADPKWELSRARLTLGKPLGEGCFGQVVMAEAIGIDKDRAA
KPVTVAVKMLKDDATDKDLSDLVSEMEMMKMIGKHKNIINLLGACTQGGP
LYVLVEYAAKGNLREFLRARRPPGLDYSFDTCKPPEEQLTFKDLVSCAYQ
VARGMEYLASQKCIHRDLAARNVLVTEDNVMKIADFGLARDVHNLDYYKK
TTNGRLPVKWMAPEALFDRVYTHQSDVWSFGVLLWEIFTLGGSPYPGIPV
EELFKLLKEGHRMDKPANCTHDLYMIMRECWHAAPSQRPTFKQLVEDLDR
VLTVTSTDVKATQEENRELRSRCEELHGKNLELGKIMDRFEEVVYQAMEE
VQKQKELSKAEIQKVLKEKDQLTTDLNSMEKSFSDLFKRFEKQKEVIEGY
RKNEESLKKCVEDYLARITQEGQRYQALKAHAEEKLQLANEEIAQVRSKA
QAEALALQASLRKEQMRIQSLEKTVEQKTKENEELTRICDDLISKMEKI

SEQ ID NO: 539 is the amino acid sequence of FGFR3ex17-TACC3ex11. The sequence corresponding to FGFR3 is underlined. The sequence corresponding to TACC3 is italicized:

MGAPACALALCVAVAIVAGASSESLGTEQRVVGRAAEVPGPEPGQQEQLV
FGSGDAVELSCPPPGGGPMGPTVWVKDGTGLVPSERVLVGPQRLQVLNAS
HEDSGAYSCRQRLTQRVLCHFSVRVTDAPSSGDDEDGEDEAEDTGVDTGA
PYWTRPERMDKKLLAVPAANTVRFRCPAAGNPTPSISWLKNGREFRGEHR
IGGIKLRHQQWSLVMESVVPSDRGNYTCVVENKFGSIRQTYTLDVLERSP
HRPILQAGLPANQTAVLGSDVEFHCKVYSDAQPHIQWLKHVEVNGSKVGP
DGTPYVTVLKTAGANTTDKELEVLSLHNVTFEDAGEYTCLAGNSIGFSHH
SAWLVVLPAEEELVEADEAGSVYAGILSYGVGFFLFILVVAAVTLCRLRS
PPKKGLGSPTVHKISRFPLKRQVSLESNASMSSNTPLVRIARLSSGEGPT
LANVSELELPADPKWELSRARLTLGKPLGEGCFGQVVMAEAIGIDKDRAA
KPVTVAVKMLKDDATDKDLSDLVSEMEMMKMIGKHKNIINLLGACTQGGP
LYVLVEYAAKGNLREFLRARRPPGLDYSFDTCKPPEEQLTFKDLVSCAYQ
VARGMEYLASQKCIHRDLAARNVLVTEDNVMKIADFGLARDVHNLDYYKK
TTNGRLPVKWMAPEALFDRVYTHQSDVWSFGVLLWEIFTLGGSPYPGIPV
EELFKLLKEGHRMDKPANCTHDLYMIMRECWHAAPSQRPTFKQLVEDLDR
VLTVTSTDVKATQEENRELRSRCEELHGKNLELGKIMDRFEEVVYQAMEE
VQKQKELSKAEIQKVLKEKDQLTTDLNSMEKSFSDLFKRFEKQKEVIEGY
RKNEESLKKCVEDYLARITQEGQRYQALKAHAEEKLQLANEEIAQVRSKA
QAEALALQASLRKEQMRIQSLEKTVEQKTKENEELTRICDDLISKMEKI

SEQ ID NO: 540 is the amino acid sequence of FGFR3ex17-TACC3ex8. The sequence corresponding to FGFR3 is underlined. The sequence corresponding to TACC3 is italicized:

MGAPACALALCVAVAIVAGASSESLGTEQRVVGRAAEVPGPEPGQQEQLV
FGSGDAVELSCPPPGGGPMGPTVWVKDGTGLVPSERVLVGPQRLQVLNAS
HEDSGAYSCRQRLTQRVLCHFSVRVTDAPSSGDDEDGEDEAEDTGVDTGA
PYWTRPERMDKKLLAVPAANTVRFRCPAAGNPTPSISWLKNGREFRGEHR
IGGIKLRHQQWSLVMESVVPSDRGNYTCVVENKFGSIRQTYTLDVLERSP
HRPILQAGLPANQTAVLGSDVEFHCKVYSDAQPHIQWLKHVEVNGSKVGP
DGTPYVTVLKTAGANTTDKELEVLSLHNVTFEDAGEYTCLAGNSIGFSHH
SAWLVVLPAEEELVEADEAGSVYAGILSYGVGFFLFILVVAAVTLCRLRS
PPKKGLGSPTVHKISRFPLKRQVSLESNASMSSNTPLVRIARLSSGEGPT
LANVSELELPADPKWELSRARLTLGKPLGEGCFGQVVMAEAIGIDKDRAA
KPVTVAVKMLKDDATDKDLSDLVSEMEMMKMIGKHKNIINLLGACTQGGP
LYVLVEYAAKGNLREFLRARRPPGLDYSFDTCKPPEEQLTFKDLVSCAYQ
VARGMEYLASQKCIHRDLAARNVLVTEDNVMKIADFGLARDVHNLDYYKK
TTNGRLPVKWMAPEALFDRVYTHQSDVWSFGVLLWEIFTLGGSPYPGIPV
EELFKLLKEGHRMDKPANCTHDLYMIMRECWHAAPSQRPTFKQLVEDLDR
VLTVTSTDFKESALRKQSLYLKFDPLLRDSPGRPVPVATETSSMHGANET
PSGRPREAKLVEFDFLGALDIPVPGPPPGVPAPGGPPLSTGPIVDLLQYS
QKDLDAVVKATQEENRELRSRCEELHGKNLELGKIMDRFEEVVYQAMEEV
QKQKELSKAEIQKVLKEKDQLTTDLNSMEKSFSDLFKRFEKQKEVIEGYR
KNEESLKKCVEDYLARITQEGQRYQALKAHAEEKLQLANEEIAQVRSKAQ
AEALALQASLRKEQMRIQSLEKTVEQKTKENEELTRICDDLISKMEKI

SEQ ID NO: 541 is the amino acid sequence of FGFR3ex17-TACC3ex10. The sequence corresponding to FGFR3 is underlined. The sequence corresponding to TACC3 is italicized:

MGAPACALALCVAVAIVAGASSESLGTEQRVVGRAAEVPGPEPGQQEQLV
FGSGDAVELSCPPPGGGPMGPTVWVKDGTGLVPSERVLVGPQRLQVLNAS
HEDSGAYSCRQRLTQRVLCHFSVRVTDAPSSGDDEDGEDEAEDTGVDTGA
PYWTRPERMDKKLLAVPAANTVRFRCPAAGNPTPSISWLKNGREFRGEHR
IGGIKLRHQQWSLVMESVVPSDRGNYTCVVENKFGSIRQTYTLDVLERSP
HRPILQAGLPANQTAVLGSDVEFHCKVYSDAQPHIQWLKHVEVNGSKVGP
DGTPYVTVLKTAGANTTDKELEVLSLHNVTFEDAGEYTCLAGNSIGFSHH
SAWLVVLPAEEELVEADEAGSVYAGILSYGVGFFLFILVVAAVTLCRLRS
PPKKGLGSPTVHKISRFPLKRQVSLESNASMSSNTPLVRIARLSSGEGPT
LANVSELELPADPKWELSRARLTLGKPLGEGCFGQVVMAEAIGIDKDRAA
KPVTVAVKMLKDDATDKDLSDLVSEMEMMKMIGKHKNIINLLGACTQGGP
LYVLVEYAAKGNLREFLRARRPPGLDYSFDTCKPPEEQLTFKDLVSCAYQ
VARGMEYLASQKCIHRDLAARNVLVTEDNVMKIADFGLARDVHNLDYYKK
TTNGRLPVKWMAPEALFDRVYTHQSDVWSFGVLLWEIFTLGGSPYPGIPV
EELFKLLKEGHRMDKPANCTHDLYMIMRECWHAAPSQRPTFKQLVEDLDR
VLTVTSTDVPGPPPGVPAPGGPPLSTGPIVDLLQYSQKDLDAVVKATQEE
NRELRSRCEELHGKNLELGKIMDRFEEVVYQAMEEVQKQKELSKAEIQKV
LKEKDQLTTDLNSMEKSFSDLFKRFEKQKEVIEGYRKNEESLKKCVEDYL
ARITQEGQRYQALKAHAEEKLQLANEEIAQVRSKAQAEALALQASLRKEQ
MRIQSLEKTVEQKTKENEELTRICDDLISKMEKI

SEQ ID NO: 542 is the amino acid sequence of FGFR3ex17-TACC3ex6. The sequence corresponding to FGFR3 is underlined. The sequence corresponding to TACC3 is italicized:

MGAPACALALCVAVAIVAGASSESLGTEQRVVGRAAEVPGPEPGQQEQLV
FGSGDAVELSCPPPGGGPMGPTVWVKDGTGLVPSERVLVGPQRLQVLNAS
HEDSGAYSCRQRLTQRVLCHFSVRVTDAPSSGDDEDGEDEAEDTGVDTGA
PYWTRPERMDKKLLAVPAANTVRFRCPAAGNPTPSISWLKNGREFRGEHR
IGGIKLRHQQWSLVMESVVPSDRGNYTCVVENKFGSIRQTYTLDVLERSP
HRPILQAGLPANQTAVLGSDVEFHCKVYSDAQPHIQWLKHVEVNGSKVGP
DGTPYVTVLKTAGANTTDKELEVLSLHNVTFEDAGEYTCLAGNSIGFSHH
SAWLVVLPAEEELVEADEAGSVYAGILSYGVGFFLFILVVAAVTLCRLRS
PPKKGLGSPTVHKISRFPLKRQVSLESNASMSSNTPLVRIARLSSGEGPT
LANVSELELPADPKWELSRARLTLGKPLGEGCFGQVVMAEAIGIDKDRAA
KPVTVAVKMLKDDATDKDLSDLVSEMEMMKMIGKHKNIINLLGACTQGGP
LYVLVEYAAKGNLREFLRARRPPGLDYSFDTCKPPEEQLTFKDLVSCAYQ
VARGMEYLASQKCIHRDLAARNVLVTEDNVMKIADFGLARDVHNLDYYKK
TTNGRLPVKWMAPEALFDRVYTHQSDVWSFGVLLWEIFTLGGSPYPGIPV
EELFKLLKEGHRMDKPANCTHDLYMIMRECWHAAPSQRPTFKQLVEDLDR
VLTVTSTDERALNSASTSLPTSCPGSEPVPTHQQGQPALELKEESFRDPA
EVLGTGAEVDYLEQFGTSSFKESALRKQSLYLKFDPLLRDSPGRPVPVAT
ETSSMHGANETPSGRPREAKLVEFDFLGALDIPVPGPPPGVPAPGGPPLS
TGPIVDLLQYSQKDLDAVVKATQEENRELRSRCEELHGKNLELGKIMDRF
EEVVYQAMEEVQKQKELSKAEIQKVLKEKDQLTTDLNSMEKSFSDLFKRF
EKQKEVIEGYRKNEESLKKCVEDYLARITQEGQRYQALKAHAEEKLQLA

SEQ ID NO: 543 is the amino acid sequence of FGFR3ex18-TACC3ex13. The sequence corresponding to FGFR3 is underlined. The sequence corresponding to TACC3 is italicized:

MGAPACALALCVAVAIVAGASSESLGTEQRVVGRAAEVPGPEPGQQEQLV
FGSGDAVELSCPPPGGGPMGPTVWVKDGTGLVPSERVLVGPQRLQVLNAS
HEDSGAYSCRQRLTQRVLCHFSVRVTDAPSSGDDEDGEDEAEDTGVDTGA
PYWTRPERMDKKLLAVPAANTVRFRCPAAGNPTPSISWLKNGREFRGEHR
IGGIKLRHQQWSLVMESVVPSDRGNYTCVVENKFGSIRQTYTLDVLERSP
HRPILQAGLPANQTAVLGSDVEFHCKVYSDAQPHIQWLKHVEVNGSKVGP
DGTPYVTVLKTAGANTTDKELEVLSLHNVTFEDAGEYTCLAGNSIGFSHH
SAWLVVLPAEEELVEADEAGSVYAGILSYGVGFFLFILVVAAVTLCRLRS
PPKKGLGSPTVHKISRFPLKRQVSLESNASMSSNTPLVRIARLSSGEGPT
LANVSELELPADPKWELSRARLTLGKPLGEGCFGQVVMAEAIGIDKDRAA
KPVTVAVKMLKDDATDKDLSDLVSEMEMMKMIGKHKNIINLLGACTQGGP
LYVLVEYAAKGNLREFLRARRPPGLDYSFDTCKPPEEQLTFKDLVSCAYQ
VARGMEYLASQKCIHRDLAARNVLVTEDNVMKIADFGLARDVHNLDYYKK
TTNGRLPVKWMAPEALFDRVYTHQSDVWSFGVLLWEIFTLGGSPYPGIPV
EELFKLLKEGHRMDKPANCTHDLYMIMRECWHAAPSQRPTFKQLVEDLDR
VLTVTSTDEYLDLSAPFEQYSPGGQDTPSKQKELSKAEIQKVLKEKDQLT
TDLNSMEKSFSDLFKRFEKQKEVIEGYRKNEESLKKCVEDYLARITQEGQ
RYQALKAHAEEKLQLANEEIAQVRSKAQAEALALQASLRKEQMRIQSLEK
TVEQKTKENEELTRICDDLISKMEKI

SEQ ID NO: 544 is the amino acid sequence of FGFR3ex18-TACC3ex9_INS66BP. The sequence corresponding to FGFR3 is underlined. The sequence corresponding to TACC3 is italicized. The sequence corresponding the the 66 bp intronic insert is double underlined:

MGAPACALALCVAVAIVAGASSESLGTEQRVVGRAAEVPGPEPGQQEQLV
FGSGDAVELSCPPPGGGPMGPTVWVKDGTGLVPSERVLVGPQRLQVLNAS
HEDSGAYSCRQRLTQRVLCHFSVRVTDAPSSGDDEDGEDEAEDTGVDTGA
PYWTRPERMDKKLLAVPAANTVRFRCPAAGNPTPSISWLKNGREFRGEHR
IGGIKLRHQQWSLVMESVVPSDRGNYTCVVENKFGSIRQTYTLDVLERSP
HRPILQAGLPANQTAVLGSDVEFHCKVYSDAQPHIQWLKHVEVNGSKVGP
DGTPYVTVLKTAGANTTDKELEVLSLHNVTFEDAGEYTCLAGNSIGFSHH
SAWLVVLPAEEELVEADEAGSVYAGILSYGVGFFLFILVVAAVTLCRLRS
PPKKGLGSPTVHKISRFPLKRQVSLESNASMSSNTPLVRIARLSSGEGPT
LANVSELELPADPKWELSRARLTLGKPLGEGCFGQVVMAEAIGIDKDRAA
KPVTVAVKMLKDDATDKDLSDLVSEMEMMKMIGKHKNIINLLGACTQGGP
LYVLVEYAAKGNLREFLRARRPPGLDYSFDTCKPPEEQLTFKDLVSCAYQ
VARGMEYLASQKCIHRDLAARNVLVTEDNVMKIADFGLARDVHNLDYYKK
TTNGRLPVKWMAPEALFDRVYTHQSDVWSFGVLLWEIFTLGGSPYPGIPV
EELFKLLKEGHRMDKPANCTHDLYMIMRECWHAAPSQRPTFKQLVEDLDR
VLTVTSTDEYLDLSAQEQRQPTLQPQGCCLAGYSHRSSMHGANETPSGRP
REAKLVEFDFLGALDIPVPGPPPGVPAPGGPPLSTGPIVDLLQYSQKDLD
AVVKATQEENRELRSRCEELHGKNLELGKIMDRFEEVVYQAMEEVQKQKE
LSKAEIQKVLKEKDQLTTDLNSMEKSFSDLFKRFEKQKEVIEGYRKNEES
LKKCVEDYLARITQEGQRYQALKAHAEEKLQLANEEIAQVRSKAQAEALA
LQASLRKEQMRIQSLEKTVEQKTKENEELTRICDDLISKMEKI

SEQ ID NO: 545 is the amino acid sequence of FGFR3ex18-TACC3ex5. The sequence corresponding to FGFR3 is underlined. The sequence corresponding to TACC3 is italicized:

MGAPACALALCVAVAIVAGASSESLGTEQRVVGRAAEVPGPEPGQQEQLV
FGSGDAVELSCPPPGGGPMGPTVWVKDGTGLVPSERVLVGPQRLQVLNAS
HEDSGAYSCRQRLTQRVLCHFSVRVTDAPSSGDDEDGEDEAEDTGVDTGA
PYWTRPERMDKKLLAVPAANTVRFRCPAAGNPTPSISWLKNGREFRGEHR
IGGIKLRHQQWSLVMESVVPSDRGNYTCVVENKFGSIRQTYTLDVLERSP
HRPILQAGLPANQTAVLGSDVEFHCKVYSDAQPHIQWLKHVEVNGSKVGP
DGTPYVTVLKTAGANTTDKELEVLSLHNVTFEDAGEYTCLAGNSIGFSHH
SAWLVVLPAEEELVEADEAGSVYAGILSYGVGFFLFILVVAAVTLCRLRS
PPKKGLGSPTVHKISRFPLKRQVSLESNASMSSNTPLVRIARLSSGEGPT
LANVSELELPADPKWELSRARLTLGKPLGEGCFGQVVMAEAIGIDKDRAA
KPVTVAVKMLKDDATDKDLSDLVSEMEMMKMIGKHKNIINLLGACTQGGP
LYVLVEYAAKGNLREFLRARRPPGLDYSFDTCKPPEEQLTFKDLVSCAYQ
VARGMEYLASQKCIHRDLAARNVLVTEDNVMKIADFGLARDVHNLDYYKK
TTNGRLPVKWMAPEALFDRVYTHQSDVWSFGVLLWEIFTLGGSPYPGIPV
EELFKLLKEGHRMDKPANCTHDLYMIMRECWHAAPSQRPTFKQLVEDLDR
VLTVTSTDEYLDLSAPVVQLAAETPTAESKERALNSASTSLPTSCPGSEP
VPTHQQGQPALELKEESFRDPAEVLGTGAEVDYLEQFGTSSFKESALRKQ
SLYLKFDPLLRDSPGRPVPVATETSSMHGANETPSGRPREAKLVEFDFLG
ALDIPVPGPPPGVPAPGGPPLSTGPIVDLLQYSQKDLDAVVKATQEENRE
LRSRCEELHGKNLELGKIMDRFEEVVYQAMEEVQKQKELSKAEIQKVLKE
KDQLTTDLNSMEKSFSDLFKRFEKQKEVIEGYRKNEESLKKCVEDYLARI
TQEGQRYQALKAHAEEKLQLANEEIAQVRSKAQAEALALQASLRKEQMRI
QSLEKTVEQKTKENEELTRICDDLISKMEKI

SEQ ID NO: 546 is the amino acid sequence of FGFR3ex18-TACC3ex5_INS33 bp. The sequence corresponding to FGFR3 is underlined. The sequence corresponding to TACC3 is italicized. The sequence corresponding the the 33 bp intronic insert is double underlined:

MGAPACALALCVAVAIVAGASSESLGTEQRVVGRAAEVPGPEPGQQEQLV
FGSGDAVELSCPPPGGGPMGPTVWVKDGTGLVPSERVLVGPQRLQVLNAS
HEDSGAYSCRQRLTQRVLCHFSVRVTDAPSSGDDEDGEDEAEDTGVDTGA
PYWTRPERMDKKLLAVPAANTVRFRCPAAGNPTPSISWLKNGREFRGEHR
IGGIKLRHQQWSLVMESVVPSDRGNYTCVVENKFGSIRQTYTLDVLERSP
HRPILQAGLPANQTAVLGSDVEFHCKVYSDAQPHIQWLKHVEVNGSKVGP
DGTPYVTVLKTAGANTTDKELEVLSLHNVTFEDAGEYTCLAGNSIGFSHH
SAWLVVLPAEEELVEADEAGSVYAGILSYGVGFFLFILVVAAVTLCRLRS
PPKKGLGSPTVHKISRFPLKRQVSLESNASMSSNTPLVRIARLSSGEGPT
LANVSELELPADPKWELSRARLTLGKPLGEGCFGQVVMAEAIGIDKDRAA
KPVTVAVKMLKDDATDKDLSDLVSEMEMMKMIGKHKNIINLLGACTQGGP
LYVLVEYAAKGNLREFLRARRPPGLDYSFDTCKPPEEQLTFKDLVSCAYQ
VARGMEYLASQKCIHRDLAARNVLVTEDNVMKIADFGLARDVHNLDYYKK
TTNGRLPVKWMAPEALFDRVYTHQSDVWSFGVLLWEIFTLGGSPYPGIPV
EELFKLLKEGHRMDKPANCTHDLYMIMRECWHAAPSQRPTFKQLVEDLDR
VLTVTSTDEYLDLSAPFEQYSPGGQDTPSSSSSGVREPPHPAFPXSAEDT
PVVQLAAETPTAESKERALNSASTSLPTSCPGSEPVPTHQQGQPALELKE
ESFRDPAEVLGTGAEVDYLEQFGTSSFKESALRKQSLYLKFDPLLRDSPG
RPVPVATETSSMHGANETPSGRPREAKLVEFDFLGALDIPVPGPPPGVPA
PGGPPLSTGPIVDLLQYSQKDLDAVVKATQEENRELRSRCEELHGKNLEL
GKIMDRFEEVVYQAMEEVQKQKELSKAEIQKVLKEKDQLTTDLNSMEKSF
SDLFKRFEKQKEVIEGYRKNEESLKKCVEDYLARITQEGQRYQALKAHAE
EKLQLANEEIAQVRSKAQAEALALQASLRKEQMRIQSLEKTVEQKTKENE
ELTRICDDLISKMEKI

SEQ ID NO: 547 is the amino acid sequence of FGFR3ex18-TACC3ex4. The sequence corresponding to FGFR3 is underlined. The sequence corresponding to TACC3 is italicized.

MGAPACALALCVAVAIVAGASSESLGTEQRVVGRAAEVPGPEPGQQEQLV
FGSGDAVELSCPPPGGGPMGPTVWVKDGTGLVPSERVLVGPQRLQVLNAS
HEDSGAYSCRQRLTQRVLCHFSVRVTDAPSSGDDEDGEDEAEDTGVDTGA
PYWTRPERMDKKLLAVPAANTVRFRCPAAGNPTPSISWLKNGREFRGEHR
IGGIKLRHQQWSLVMESVVPSDRGNYTCVVENKFGSIRQTYTLDVLERSP
HRPILQAGLPANQTAVLGSDVEFHCKVYSDAQPHIQWLKHVEVNGSKVGP
DGTPYVTVLKTAGANTTDKELEVLSLHNVTFEDAGEYTCLAGNSIGFSHH
SAWLVVLPAEEELVEADEAGSVYAGILSYGVGFFLFILVVAAVTLCRLRS
PPKKGLGSPTVHKISRFPLKRQVSLESNASMSSNTPLVRIARLSSGEGPT
LANVSELELPADPKWELSRARLTLGKPLGEGCFGQVVMAEAIGIDKDRAA
KPVTVAVKMLKDDATDKDLSDLVSEMEMMKMIGKHKNIINLLGACTQGGP
LYVLVEYAAKGNLREFLRARRPPGLDYSFDTCKPPEEQLTFKDLVSCAYQ
VARGMEYLASQKCIHRDLAARNVLVTEDNVMKIADFGLARDVHNLDYYKK
TTNGRLPVKWMAPEALFDRVYTHQSDVWSFGVLLWEIFTLGGSPYPGIPV
EELFKLLKEGHRMDKPANCTHDLYMIMRECWHAAPSQRPTFKQLVEDLDR
VLTVTSTDEYLDLSAPFEQYSPGGQDTPESPETRLGQPAAEQLHAGPATE
EPGPCLSQQLHSASAEDTPVVQLAAETPTAESKERALNSASTSLPTSCPG
SEPVPTHQQGQPALELKEESFRDPAEVLGTGAEVDYLEQFGTSSFKESAL
RKQSLYLKFDPLLRDSPGRPVPVATETSSMHGANETPSGRPREAKLVEFD
FLGALDIPVPGPPPGVPAPGGPPLSTGPIVDLLQYSQKDLDAVVKATQEE
NRELRSRCEELHGKNLELGKIMDRFEEVVYQAMEEVQKQKELSKAEIQKV
LKEKDQLTTDLNSMEKSFSDLFKRFEKQKEVIEGYRKNEESLKKCVEDYL
ARITQEGQRYQALKAHAEEKLQLANEEIAQVRSKAQAEALALQASLRKEQ
MRIQSLEKTVEQKTKENEELTRICDDLISKMEKI

The Genbank ID for the FGFR4 gene is 2264. Three isoforms are listed for FGFR4, e.g., having Genebank Accession Nos. NP_002002 (corresponding nucleotide sequence NM_002011); NP_075252 (corresponding nucleotide sequence NM_022963); NP_998812 (corresponding nucleotide sequence NM_213647).

As used herein, a “FGFR fusion molecule” can be a nucleic acid (e.g., synthetic, purified, and/or recombinant) which encodes a polypeptide corresponding to a fusion protein comprising a tyrosine kinase domain of an FGFR protein fused to a polypeptide that constitutively activates the tyrosine kinase domain of the FGFR protein, or a nucleic acid encoding a fusion protein comprising a transforming acidic coiled-coil (TACC) domain fused to a polypeptide with a tyrosine kinase domain, wherein the TACC domain constitutively activates the tyrosine kinase domain. It can also be a fusion protein comprising a tyrosine kinase domain of an FGFR protein fused to a polypeptide that constitutively activates the tyrosine kinase domain of the FGFR protein, or a fusion protein comprising a transforming acidic coiled-coil (TACC) domain fused to a polypeptide with a tyrosine kinase domain, wherein the TACC domain constitutively activates the tyrosine kinase domain. For example, a FGFR fusion molecule can include a FGFR1-TACC1 (e.g., comprising the amino acid sequence shown in SEQ ID NO: 150, or comprising the nucleic acid sequence shown in SEQ ID NO: 88), FGFR2-TACC2, FGFR3-TACC3 (e.g., comprising the amino acid sequence shown in SEQ ID NOS: 79, 158-161, or 539-547 or comprising the nucleic acid sequence shown in SEQ ID NOS: 80-82, 84, 94-145, 515, 517, 519-527, or 530-538), or other FGFR-TACC fusion proteins (e.g., an N-terminal fragment of FGFR4 containing its tyrosine kinase domain fused to a fragment of TACC1, TACC2, or TACC3). For example, a FGFR fusion molecule can include a FGFR1-containing fusion comprising the amino acid sequence corresponding to Genebank Accession no. NP_001167534, NP_001167535, NP_001167536, NP_001167537, NP_001167538, NP_056934, NP_075593, NP_075594, or NP_075598; or a FGFR1-containing fusion comprising the nucleotide sequence corresponding to Genebank Accession no. NM_001174063, NM_001174064, NM_001174065, NM_001174066, NM_001174067, NM_015850, NM_023105, NM_023106, or NM_023110. For example, a FGFR fusion molecule can include a FGFR2-containing fusion comprising the amino acid sequence corresponding to Genebank Accession no. NP_000132, NP_001138385, NP_001138386, NP_001138387, NP_001138388, NP_001138389, NP_001138390, NP_001138391, or NP_075259; or a FGFR2-containing fusion comprising the nucleotide sequence corresponding to Genebank Accession no. NM_000141, NM_001144913, NM_001144914, NM_001144915, NM_001144916, NM_001144917, NM_001144918, NM_001144919, or NM_022970. For example, a FGFR fusion molecule can include a FGFR3-containing fusion comprising the amino acid sequence corresponding to Genebank Accession no. NP_000133, NP_001156685, or NP_075254; or a FGFR3-containing fusion comprising the nucleotide sequence corresponding to Genebank Accession no. NM_000142, NM_001163213, or NM_022965. For example, a FGFR fusion molecule can include a FGFR4-containing fusion comprising the amino acid sequence corresponding to Genebank Accession no. NP_002002, NP_075252, or NP_998812; or a FGFR4-containing fusion comprising the nucleotide sequence corresponding to Genebank Accession no. NM_002011, NM_022963, or NM_213647. A FGFR fusion molecule can also include a tyrosine kinase domain of an FGFR protein fused to a protein encoded by any one of the genes listed in Table 1A. A FGFR fusion molecule can include a variant of the above described examples, such as a fragment thereof.

TABLE 1A
Fusion Partners

| gene | gene | gene | gene |
|---|---|---|---|
| ABCA13 | C21orf29 | CAMKK1 | DNAJC6 |
| ABCC1 | CACNA1C | CAMSAP1 | DYRK3 |
| ABCC12 | CACNA1G | CAMTA1 | EIF2C2 |
| ABCC6 | CNTNAP4 | CAP2 | FAM184B |
| ABL1 | CUL3 | CCDC147 | FREM2 |
| ADAM12 | DMD | CCDC158 | GDPD2 |
| ADCY10 | DUSP27 | CELF2 | GLI3 |
| ADCY2 | ECE1 | CILP | IL1RN |
| ADCY8 | EYS | CMYA5 | ISX |
| AGBL4 | FAM172A | COL14A1 | KIDINS220 |
| AHNAK | FAM184B | CORO7 | LRBA |
| ANXA7 | FGFR4 | CSMD2 | LY75 |
| AP4S1 | ITGAV | CUL3 | MDH2 |
| AQP2 | LRP1 | DDI2 | MMP12 |
| ARMC6 | LY75 | DEPDC5 | N4BP2L2 |
| ATP5B | MAPKAP1 | DEPDC7 | NCF2 |
| ATP6AP1L | MYT1 | DI10L | NCOR1 |
| ATP6V0D2 | NCF2 | DMD | NCRNA00157 |
| ATXN1 | NCOR1 | EDA | NRXN3 |
| BAHD1 | NHSL2 | EFHC1 | PARP16 |
| BBX | NKAIN2 | EFS | PLA2G2F |
| BCA10 | NR3C1 | EIF2C2 | PLEK2 |
| C15orf23 | NUP188 | ENTPD2 | PRKCH |
| C15orf33 | OSBPL10 | EYS | PTPRS |
| C21orf29 | PACSIN1 | FAM160A1 | ROBO1 |
| C2CD3 | PARP16 | MUSK | SASH3 |
| C6orf170 | PDZRN4 | NEUROG1 | SH3BP5 |
| C7orf44 | POLM | NHSL2 | SLC44A2 |
| CACNA1C | PPP1R3A | NR3C1 | SLC5A4 |
| CACNA1G | PSEN1 | ODZ1 | SNX5 |
| FAM168A | PTPRD | PCDH12 | SORCS2 |
| FAM172A | PTPRS | PLCL1 | SRRM1 |
| FAM192A | RALYL | PLEKHM3 | SSX3 |
| FAM19A2 | RERE | PLOD3 | STAG2 |
| FBXL4 | RIMBP2 | PRKCH | STK24 |
| FH | RNF216 | PSEN1 | SURF6 |
| FREM2 | SDAD1 | SEPT5 | SYNPO2 |
| GAPVD1 | SEC14L3 | SLC44A2 | TAF1 |
| GLI3 | SH3RF3 | SNTA1 | TMEM80 |
| GPR182 | SLC9A1 | USP48 | TNFRSF10B |
| GSTA3 | SMOC2 | VSNL1 | TTYH1 |
| IGFBP3 | SNX5 | WDFY1 | UNC93B1 |
| ITGA9 | TACC2 | WISP2 | VSNL1 |
| ITGB2 | SRGAP1 | XRRA1 | XRCC4 |
| JOSD2 | SSX3 | LRRC4B | ZNF410 |
| KIDINS220 | SUMF1 | LRRK2 | TRIOBP |
| LAMA2 | SYNPO2 | MAPKAP1 | TTYH1 |
| LCLAT1 | TNFRSF10B | MST1R | LRBA |
| LIN9 | | | |

For example, a FGFR fusion molecule can include a FGFR3-containing fusion comprising the amino acid sequence corresponding to residues 1-760 of FGFR3 (e.g. SEQ ID NO: 90) fused to the amino acid sequence corresponding residues 648-838 of TACC3 (e.g. SEQ ID NO: 92). A FGFR fusion molecule can also include a FGFR3-containing fusion comprising the amino acid sequence corresponding to residues 1-760 of FGFR3 (e.g. SEQ ID NO: 90) fused to the amino acid sequence corresponding residues 549-838 of TACC3 (e.g. SEQ ID NO: 92). A FGFR fusion molecule can include a FGFR3-containing fusion comprising the amino acid sequence corresponding to residues 1-760 of FGFR3 (e.g. SEQ ID NO: 90) fused to the amino acid sequence corresponding residues 613-838 of TACC3 (e.g. SEQ ID NO: 92). A FGFR fusion molecule can include a FGFR3-containing fusion comprising the amino acid sequence corresponding to residues 1-760 of FGFR3 (e.g. SEQ ID NO: 90) fused to the amino acid sequence corresponding residues 488-838 of TACC3 (e.g. SEQ ID NO: 92). A FGFR fusion molecule can include a FGFR3-containing fusion comprising the amino acid sequence corresponding to residues 1-781 of FGFR3 (e.g. SEQ ID NO: 90) fused to the amino acid sequence corresponding residues 689-838 of TACC3 (e.g. SEQ ID NO: 92). A FGFR fusion molecule can include a FGFR3-containing fusion comprising the amino acid sequence corresponding to residues 1-765 of FGFR3 (e.g. SEQ ID NO: 90) fused to the amino acid sequence corresponding residues 583-838 of TACC3 (e.g. SEQ ID NO: 92). A FGFR fusion molecule can include a FGFR3-containing fusion comprising the amino acid sequence corresponding to residues 1-767 of FGFR3 (e.g. SEQ ID NO: 90) fused to the amino acid sequence corresponding residues 462-838 of TACC3 (e.g. SEQ ID NO: 92). A FGFR fusion molecule can include a FGFR3-containing fusion comprising the amino acid sequence corresponding to residues 1-767 of FGFR3 (e.g. SEQ ID NO: 90) fused to the amino acid sequence corresponding residues 472-838 of TACC3 (e.g. SEQ ID NO: 92). A FGFR fusion molecule can include a FGFR3-containing fusion comprising the amino acid sequence corresponding to residues 1-780 of FGFR3 (e.g. SEQ ID NO: 90) fused to the amino acid sequence corresponding residues 432-838 of TACC3 (e.g. SEQ ID NO: 92). A FGFR fusion molecule can include a FGFR1-containing fusion comprising the amino acid sequence corresponding to residues 1-762 of FGFR1 (e.g. SEQ ID NOS: 146, 185) fused to the amino acid sequence corresponding residues 571-805 of TACC1 (e.g. SEQ ID NO: 148). For example, a FGFR fusion molecule can include SEQ ID NOs: 539-543, 545, and 547.

The alteration in a chromosome region occupied by a FGFR fusion molecule, e.g., a FGFR1-TACC1, FGFR2-TACC2, FGFR3-TACC3 or other FGFR-TACC nucleic acid, can result in amino acid substitutions, RNA splicing or processing, product instability, the creation of stop codons, production of oncogenic fusion proteins, frame-shift mutations, and/or truncated polypeptide production. A FGFR fusion molecule can include FGFR and TACC exons joined in the fused mRNA or cDNA. A FGFR fusion molecule can also include FGFR and TACC exons joined in the fused mRNA or cDNA along with the presence of FGFR or TACC introns that are spliced in the fusion cDNA. FGFR or TACC introns can encode amino acids of the FGFR fusion molecule. For example, a FGFR fusion molecule can include a FGFR3-containing fusion comprising the amino acid sequence corresponding to residues 1-765 of FGFR3 (e.g. SEQ ID NO: 90) fused to a 22 amino acid sequence encoded by a TACC3 intron fused to the amino acid sequence corresponding to residues 583-838 of TACC3 (e.g. SEQ ID NO: 92). For example, a FGFR fusion molecule can include a FGFR3-containing fusion comprising the amino acid sequence corresponding to residues 1-767 of FGFR3 (e.g. SEQ ID NO: 90) fused to a 11 amino acid sequence encoded by a TACC3 intron fused to the amino acid sequence corresponding to residues 472-838 of TACC3 (e.g. SEQ ID NO: 92). For example, a FGFR fusion molecule can include SEQ ID NOs: 544 and 546.

A FGFR fusion protein can also include a fusion protein encoded by an FGFR3-TACC3 nucleic acid, wherein FGFR3-TACC3 comprises a combination of introns and exons 1-18 of FGFR3 located on human chromosome 4p16 spliced 5′ to a combination of introns and exons 4-16 of TACC3 located on human chromosome 4p16, wherein a genomic breakpoint occurs in any one of introns or exons 1-18 of FGFR3 and any one of introns or exons 3-16 of TACC3. For example, a FGFR fusion protein can also include a fusion protein encoded by an FGFR3-TACC3 nucleic acid, wherein the nucleic acid comprises exons 1-17 of FGFR3 located on human chromosome 4p16 spliced 5′ to exons 11-16 of TACC3 located on human chromosome 4p16. In one embodiment, a genomic breakpoint occurs in intron 17 of FGFR3 and in intron 10 of TACC3. For example, a FGFR fusion protein can also include a fusion protein encoded by an FGFR3-TACC3 nucleic acid, wherein the nucleic acid comprises exons 1-17 of FGFR3 located on human chromosome 4p16 spliced 5′ to exons 8-16 of TACC3 located on human chromosome 4p16. In one embodiment, a genomic breakpoint occurs in intron 17 of FGFR3 and in intron 7 of TACC3. For example, a FGFR fusion protein can also include a fusion protein encoded by an FGFR3-TACC3 nucleic acid, wherein the nucleic acid comprises exons 1-17 of FGFR3 located on human chromosome 4p16 spliced 5′ to exons 10-16 of TACC3 located on human chromosome 4p16. In one embodiment, a genomic breakpoint occurs in exon 18 of FGFR3 and in intron 9 of TACC3. For example, a FGFR fusion protein can also include a fusion protein encoded by an FGFR3-TACC3 nucleic acid, wherein the nucleic acid comprises exons 1-17 of FGFR3 located on human chromosome 4p16 spliced 5′ to exons 6-16 of TACC3 located on human chromosome 4p16. In one embodiment, a genomic breakpoint occurs in intron 17 of FGFR3 and in intron 5 of TACC3. In one embodiment, a genomic breakpoint occurs in intron 17 of FGFR3 and in exon 5 of TACC3. For example, a FGFR fusion protein can also include a fusion protein encoded by an FGFR3-TACC3 nucleic acid, wherein the nucleic acid comprises exons 1-18 of FGFR3 located on human chromosome 4p16 spliced 5′ to exons 13-16 of TACC3 located on human chromosome 4p16. In one embodiment, a genomic breakpoint occurs in exon 18 of FGFR3 and in intron 12 or exon 13 of TACC3. For example, a FGFR fusion protein can also include a fusion protein encoded by an FGFR3-TACC3 nucleic acid, wherein the nucleic acid comprises exons 1-18 of FGFR3 located on human chromosome 4p16 spliced 5′ to a portion of intron 8 of TACC3 and exons 9-16 of TACC3 located on human chromosome 4p16. In one embodiment, a genomic breakpoint occurs in exon 18 of FGFR3 and in intron 8 of TACC3. For example, a FGFR fusion protein can also include a fusion protein encoded by an FGFR3-TACC3 nucleic acid, wherein the nucleic acid comprises exons 1-18 of FGFR3 located on human chromosome 4p16 spliced 5′ to exons 5-16 of TACC3 located on human chromosome 4p16. In one embodiment, a genomic breakpoint occurs in exon 18 of FGFR3 and in intron 4 or exon 5 of TACC3. For example, a FGFR fusion protein can also include a fusion protein encoded by an FGFR3-TACC3 nucleic acid, wherein the nucleic acid comprises exons 1-18 of FGFR3 located on human chromosome 4p16 spliced 5′ to a portion of intron 4 of TACC 3 and exons 5-16 of TACC3 located on human chromosome 4p16. In one embodiment, a genomic breakpoint occurs in exon 18 of FGFR3 and in intron 4 or exon 5 of TACC3. For example, a FGFR fusion protein can also include a fusion protein encoded by an FGFR3-TACC3 nucleic acid, wherein the nucleic acid comprises exons 1-18 of FGFR3 located on human chromosome 4p16 spliced 5′ to exons 4-16 of TACC3 located on human chromosome 4p16. In one embodiment, a genomic breakpoint occurs in exon 18 of FGFR3 and in intron 3 or exon 4 of TACC3. For example, a FGFR fusion protein can also include a fusion protein encoded by an FGFR3-TACC3 nucleic acid, wherein the nucleic acid comprises exons 1-17 of FGFR3 and a portion of intron 17 of FGFR3 located on human chromosome 4p16 spliced 5′ to exons 9-16 of TACC3 located on human chromosome 4p16. In one embodiment, a genomic breakpoint occurs in intron 17 of FGFR3 and in exon 9 of TACC3. For example, a FGFR fusion protein can also include a fusion protein encoded by an FGFR1-TACC1 nucleic acid, wherein the nucleic acid comprises exons 1-17 of FGFR1 located on human chromosome 8p11 spliced 5′ to exons 7-13 of TACC1 located on human chromosome 8p11. In one embodiment, a genomic breakpoint occurs in intron 17 or exon 17 of FGFR1 and in intron 6 or exon 7 of TACC1.

In one embodiment, the coordinates comprising FGFR3 translocations comprise chr4:1,795,039-1,810,599. In a further embodiment, the genomic breakpoint coordinate according to the genome build GRCh37/hg19 for FGFR3 is chr4:1,808,808, chr4:1,808,843, chr4:1,809,083, chr4:1,808,785, chr4:1,808,700, chr4:1,808,864, chr4:1,808,678, chr4:1, 808,798, or chr4:1,808,723. In a further embodiment, the coordinates comprising TACC3 translocations comprise chr4:1,723,217-1,746,905. In a further embodiment, the genomic breakpoint coordinate according to the genome build GRCh37/hg19 for TACC3 is chr4:1,732,648, chr4:1,732,757, chr4:1,739,187, chr4:1,737,091, chr4:1,737,062, chr4:1,737741, chr4:1,739,662, chr4:1,739,600, or chr4:1,738,989.

The nucleic acid can be any type of nucleic acid, including genomic DNA, complementary DNA (cDNA), recombinant DNA, synthetic or semi-synthetic DNA, as well as any form of corresponding RNA. A cDNA is a form of DNA artificially synthesized from a messenger RNA template and is used to produce gene clones. A synthetic DNA is free of modifications that can be found in cellular nucleic acids, including, but not limited to, histones and methylation. For example, a nucleic acid encoding a FGFR fusion molecule can comprise a recombinant nucleic acid encoding such a protein. The nucleic acid can be a non-naturally occurring nucleic acid created artificially (such as by assembling, cutting, ligating or amplifying sequences). It can be double-stranded or single-stranded.

The invention further provides for nucleic acids that are complementary to a FGFR fusion molecule. Complementary nucleic acids can hybridize to the nucleic acid sequence described above under stringent hybridization conditions. Non-limiting examples of stringent hybridization conditions include temperatures above 30° C., above 35° C., in excess of 42° C., and/or salinity of less than about 500 mM, or less than 200 mM. Hybridization conditions can be adjusted by the skilled artisan via modifying the temperature, salinity and/or the concentration of other reagents such as SDS or SSC.

According to the invention, protein variants can include amino acid sequence modifications. For example, amino acid sequence modifications fall into one or more of three classes: substitutional, insertional or deletional variants. Insertions can include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. These variants ordinarily are prepared by site-specific mutagenesis of nucleotides in the DNA encoding the protein, thereby producing DNA encoding the variant, and thereafter expressing the DNA in recombinant cell culture.

In one embodiment, a FGFR fusion molecule comprises a protein or polypeptide encoded by a nucleic acid sequence encoding a FGFR fusion molecule, such as the sequences shown in SEQ ID NOS: 80-82, 84, 94-145, 515, 517, 519-527, or 530-538. In some embodiments, the nucleic acid sequence encoding a FGFR fusion molecule is about 70%, about 75%, about 80%, about 85%, about 90%, about 93%, about 95%, about 97%, about 98%, or about 99% identical to SEQ ID NOS: 80-82, 84, 94-145, 515, 517, 519-527, or 530-538. In another embodiment, the polypeptide can be modified, such as by glycosylations and/or acetylations and/or chemical reaction or coupling, and can contain one or several non-natural or synthetic amino acids. An example of a FGFR fusion molecule is the polypeptide having the amino acid sequence shown in SEQ ID NOS: 79, 88, 150, 158-161, or 539-547. In some embodiments, the FGFR fusion molecule that is a polypeptide is about 70%, about 75%, about 80%, about 85%, about 90%, about 93%, about 95%, about 97%, about 98%, or about 99% identical to SEQ ID NOS: 79, 88, 150, 158-161, or 539-547. In another embodiment, a FGFR fusion molecule can be a fragment of a FGFR fusion protein. For example, the FGFR fusion molecule can encompass any portion of at least about 8 consecutive amino acids of SEQ ID NOS: 79, 88, 150, 158-161, or 539-547. The fragment can comprise at least about 10 amino acids, a least about 20 amino acids, at least about 30 amino acids, at least about 40 amino acids, a least about 50 amino acids, at least about 60 amino acids, or at least about 75 amino acids of SEQ ID NOS: 79, 88, 150, 158-161, or 539-547. Fragments include all possible amino acid lengths between about 8 and 100 about amino acids, for example, lengths between about 10 and 100 amino acids, between about 15 and 100 amino acids, between about 20 and 100 amino acids, between about 35 and 100 amino acids, between about 40 and 100 amino acids, between about 50 and 100 amino acids, between about 70 and 100 amino acids, between about 75 and 100 amino acids, or between about 80 and 100 amino acids. Fragments include all possible amino acid lengths between about 100 and 800 amino acids, for example, lengths between about 125 and 800 amino acids, between about 150 and 800 amino acids, between about 175 and 800 amino acids, between about 200 and 800 amino acids, between about 225 and 800 amino acids, between about 250 and 800 amino acids, between about 275 and 800 amino acids, between about 300 and 800 amino acids, between about 325 and 800 amino acids, between about 350 and 800 amino acids, between about 375 and 800 amino acids, between about 400 and 800 amino acids, between about 425 and 800 amino acids, between about 450 and 800 amino acids, between about 475 and 800 amino acids, between about 500 and 800 amino acids, between about 525 and 800 amino acids, between about 550 and 800 amino acids, between about 575 and 800 amino acids, between about 600 and 800 amino acids, between about 625 and 800 amino acids, between about 650 and 800 amino acids, between about 675 and 800 amino acids, between about 700 and 800 amino acids, between about 725 and 800 amino acids, between about 750 and 800 amino acids, or between about 775 and 800 amino acids.

Chemical Synthesis. Nucleic acid sequences encoding a FGFR fusion molecule can be synthesized, in whole or in part, using chemical methods known in the art. Alternatively, a polypeptide can be produced using chemical methods to synthesize its amino acid sequence, such as by direct peptide synthesis using solid-phase techniques. Protein synthesis can either be performed using manual techniques or by automation. Automated synthesis can be achieved, for example, using Applied Biosystems 431A Peptide Synthesizer (Perkin Elmer).

Optionally, polypeptides fragments can be separately synthesized and combined using chemical methods to produce a full-length molecule. For example, these methods can be utilized to synthesize a fusion protein of the invention. In one embodiment, the fusion protein comprises a tyrosine kinase domain of an FGFR protein fused to a polypeptide that constitutively activates the tyrosine kinase domain of the FGFR protein. In another embodiment, a fusion protein comprises a transforming acidic coiled-coil (TACC) domain fused to a polypeptide with a tyrosine kinase domain, wherein the TACC domain constitutively activates the tyrosine kinase domain. An example of a fusion protein is the FGFR1-TACC1 polypeptide, which comprises the amino acid sequence shown in SEQ ID NO: 150. An example of a fusion protein is the FGFR3-TACC3 polypeptide, which has the amino acid sequence comprising SEQ ID NO: 79, 158, 159, 160, 161,539, 540, 541, 542, 543, 544, 545, 546, or 547.

Obtaining, Purifying and Detecting FGFR fusion molecules. A polypeptide encoded by a nucleic acid, such as a nucleic acid encoding a FGFR fusion molecule, or a variant thereof, can be obtained by purification from human cells expressing a protein or polypeptide encoded by such a nucleic acid. Non-limiting purification methods include size exclusion chromatography, ammonium sulfate fractionation, ion exchange chromatography, affinity chromatography, and preparative gel electrophoresis.

A synthetic polypeptide can be substantially purified via high performance liquid chromatography (HPLC), such as ion exchange chromatography (IEX-HPLC). The composition of a synthetic polypeptide, such as a FGFR fusion molecule, can be confirmed by amino acid analysis or sequencing.

Other constructions can also be used to join a nucleic acid sequence encoding a polypeptide/protein of the claimed invention to a nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). Including cleavable linker sequences (i.e., those specific for Factor Xa or enterokinase (Invitrogen, San Diego, Calif.)) between the purification domain and a polypeptide encoded by a nucleic acid of the invention also can be used to facilitate purification. For example, the skilled artisan can use an expression vector encoding 6 histidine residues that precede a thioredoxin or an enterokinase cleavage site in conjunction with a nucleic acid of interest. The histidine residues facilitate purification by immobilized metal ion affinity chromatography, while the enterokinase cleavage site provides a means for purifying the polypeptide encoded by, for example, an FGFR1-TACC1, FGFR2-TACC2, FGFR3-TACC3, other FGFR-TACC, FGFR-containing, or TACC containing nucleic acid.

Host cells which contain a nucleic acid encoding a FGFR fusion molecule, and which subsequently express the same, can be identified by various procedures known to those of skill in the art. These procedures include, but are not limited to, DNA-DNA or DNA-RNA hybridizations and protein bioassay or immunoassay techniques which include membrane, solution, or chip-based technologies for the detection and/or quantification of nucleic acid or protein. For example, the presence of a nucleic acid encoding a FGFR fusion molecule can be detected by DNA-DNA or DNA-RNA hybridization or amplification using probes or fragments of nucleic acids encoding the same. In one embodiment, a nucleic acid fragment of a FGFR fusion molecule can encompass any portion of at least about 8 consecutive nucleotides of SEQ ID NOS: 80-82, 84, 94-145, 515, 517, 519-527, or 530-538. In another embodiment, the fragment can comprise at least about 10 consecutive nucleotides, at least about 15 consecutive nucleotides, at least about 20 consecutive nucleotides, or at least about 30 consecutive nucleotides of SEQ ID NOS: 80-82, 84, 94-145, 515, 517, 519-527, 530-538. Fragments can include all possible nucleotide lengths between about 8 and about 100 nucleotides, for example, lengths between about 15 and about 100 nucleotides, or between about 20 and about 100 nucleotides. Nucleic acid amplification-based assays involve the use of oligonucleotides selected from sequences encoding a FGFR fusion molecule nucleic acid, or FGFR fusion molecule nucleic acid to detect transformants which contain a nucleic acid encoding a protein or polypeptide of the same.

Protocols are known in the art for detecting and measuring the expression of a polypeptide encoded by a nucleic acid, such as a nucleic acid encoding a FGFR fusion molecule, using either polyclonal or monoclonal antibodies specific for the polypeptide. Non-limiting examples include enzyme-linked immunosorbent assay (ELISA), radioimmunoassay (RIA), immunostaining, and fluorescence activated cell sorting (FACS). A two-site, monoclonal-based immunoassay using monoclonal antibodies reactive to two non-interfering epitopes on a polypeptide encoded by a nucleic acid, such as a nucleic acid encoding a FGFR fusion molecule, can be used, or a competitive binding assay can be employed.

Labeling and conjugation techniques are known by those skilled in the art and can be used in various nucleic acid and amino acid assays. Methods for producing labeled hybridization or PCR probes for detecting sequences related to nucleic acid sequences encoding a protein, such as FGFR fusion molecule, include, but are not limited to, oligolabeling, nick translation, end-labeling, or PCR amplification using a labeled nucleotide. Alternatively, nucleic acid sequences, such as nucleic acids encoding a FGFR fusion molecule, can be cloned into a vector for the production of an mRNA probe. Such vectors are known in the art, are commercially available, and can be used to synthesize RNA probes in vitro by addition of labeled nucleotides and an appropriate RNA polymerase such as T7, T3, or SP6. These procedures can be conducted using a variety of commercially available kits (Amersham Pharmacia Biotech, Promega, and US Biochemical). Suitable reporter molecules or labels which can be used for ease of detection include radionuclides, enzymes, and fluorescent, chemiluminescent, or chromogenic agents, as well as substrates, cofactors, inhibitors, and/or magnetic particles.

A fragment can be a fragment of a protein, such as a FGFR fusion protein. For example, a fragment of a FGFR fusion molecule can encompass any portion of at least about 8 consecutive amino acids of SEQ ID NOS: 79, 88, 150, 158-161, or 539-547. The fragment can comprise at least about 10 consecutive amino acids, at least about 20 consecutive amino acids, at least about 30 consecutive amino acids, at least about 40 consecutive amino acids, a least about 50 consecutive amino acids, at least about 60 consecutive amino acids, at least about 70 consecutive amino acids, at least about 75 consecutive amino acids, at least about 80 consecutive amino acids, at least about 85 consecutive amino acids, at least about 90 consecutive amino acids, at least about 95 consecutive amino acids, at least about 100 consecutive amino acids, at least about 200 consecutive amino acids, at least about 300 consecutive amino acids, at least about 400 consecutive amino acids, at least about 500 consecutive amino acids, at least about 600 consecutive amino acids, at least about 700 consecutive amino acids, or at least about 800 consecutive amino acids of SEQ ID NOS: 79, 88, 150, 158-161, or 539-547. Fragments include all possible amino acid lengths between about 8 and 100 about amino acids, for example, lengths between about 10 and about 100 amino acids, between about 15 and about 100 amino acids, between about 20 and about 100 amino acids, between about 35 and about 100 amino acids, between about 40 and about 100 amino acids, between about 50 and about 100 amino acids, between about 70 and about 100 amino acids, between about 75 and about 100 amino acids, or between about 80 and about 100 amino acids.

### Cell Transfection

Host cells transformed with a nucleic acid sequence of interest can be cultured under conditions suitable for the expression and recovery of the protein from cell culture. The polypeptide produced by a transformed cell can be secreted or contained intracellularly depending on the sequence and/or the vector used. Expression vectors containing a nucleic acid sequence, such as a nucleic acid encoding a FGFR fusion molecule, can be designed to contain signal sequences which direct secretion of soluble polypeptide molecules encoded by the nucleic acid. Cell transfection and culturing methods are described in more detail below.

A eukaryotic expression vector can be used to transfect cells in order to produce proteins encoded by nucleotide sequences of the vector, e.g. those encoding a FGFR fusion molecule. Mammalian cells can contain an expression vector (for example, one that contains a nucleic acid encoding a fusion protein comprising a tyrosine kinase domain of an FGFR protein fused to a polypeptide that constitutively activates the tyrosine kinase domain of the FGFR protein, or a nucleic acid encoding a fusion protein comprises a transforming acidic coiled-coil (TACC) domain fused to a polypeptide with a tyrosine kinase domain, wherein the TACC domain constitutively activates the tyrosine kinase domain) via introducing the expression vector into an appropriate host cell via methods known in the art.

A host cell strain can be chosen for its ability to modulate the expression of the inserted sequences or to process the expressed polypeptide encoded by a nucleic acid, in the desired fashion. Such modifications of the polypeptide include, but are not limited to, acetylation, carboxylation, glycosylation, phosphorylation, lipidation, and acylation. Post-translational processing which cleaves a “prepro” form of the polypeptide also can be used to facilitate correct insertion, folding and/or function. Different host cells which have specific cellular machinery and characteristic mechanisms for post-translational activities (e.g., CHO, HeLa, MDCK, HEK293, and WI38), are available from the American Type Culture Collection (ATCC; 10801 University Boulevard, Manassas, Va. 20110-2209) and can be chosen to ensure the correct modification and processing of the foreign protein.

An exogenous nucleic acid can be introduced into a cell via a variety of techniques known in the art, such as lipofection, microinjection, calcium phosphate or calcium chloride precipitation, DEAE-dextran-mediated transfection, or electroporation. Electroporation is carried out at approximate voltage and capacitance to result in entry of the DNA construct(s) into cells of interest (such as glioma cells (cell line SF188), neuroblastoma cells (cell lines IMR-32, SK-N-SH, SH-F and SH-N), astrocytes and the like). Other transfection methods also include modified calcium phosphate precipitation, polybrene precipitation, liposome fusion, and receptor-mediated gene delivery.

Cells that will be genetically engineered can be primary and secondary cells obtained from various tissues, and include cell types which can be maintained and propagated in culture. Non-limiting examples of primary and secondary cells include epithelial cells, neural cells, endothelial cells, glial cells, fibroblasts, muscle cells (such as myoblasts) keratinocytes, formed elements of the blood (e.g., lymphocytes, bone marrow cells), and precursors of these somatic cell types.

Vertebrate tissue can be obtained by methods known to one skilled in the art, such a punch biopsy or other surgical methods of obtaining a tissue source of the primary cell type of interest. In one embodiment, a punch biopsy or removal (e.g., by aspiration) can be used to obtain a source of cancer cells (for example, glioma cells, neuroblastoma cells, and the like). A mixture of primary cells can be obtained from the tissue, using methods readily practiced in the art, such as explanting or enzymatic digestion (for examples using enzymes such as pronase, trypsin, collagenase, elastase dispase, and chymotrypsin). Biopsy methods have also been described in U.S. Pat. No. 7,419,661 and PCT application publication WO 2001/32840, and each are hereby incorporated by reference.

Primary cells can be acquired from the individual to whom the genetically engineered primary or secondary cells are administered. However, primary cells can also be obtained from a donor, other than the recipient, of the same species. The cells can also be obtained from another species (for example, rabbit, cat, mouse, rat, sheep, goat, dog, horse, cow, bird, or pig). Primary cells can also include cells from a purified vertebrate tissue source grown attached to a tissue culture substrate (for example, flask or dish) or grown in a suspension; cells present in an explant derived from tissue; both of the aforementioned cell types plated for the first time; and cell culture suspensions derived from these plated cells. Secondary cells can be plated primary cells that are removed from the culture substrate and replated, or passaged, in addition to cells from the subsequent passages. Secondary cells can be passaged one or more times. These primary or secondary cells can contain expression vectors having a gene that encodes a FGFR fusion molecule.

### Cell Culturing

Various culturing parameters can be used with respect to the host cell being cultured. Appropriate culture conditions for mammalian cells are well known in the art (Cleveland W L, et al., J Immunol Methods, 1983, 56(2): 221-234) or can be determined by the skilled artisan (see, for example, Animal Cell Culture: A Practical Approach 2nd Ed., Rickwood, D. and Hames, B. D., eds. (Oxford University Press: New York, 1992)). Cell culturing conditions can vary according to the type of host cell selected. Commercially available medium can be utilized. Non-limiting examples of medium include, for example, Minimal Essential Medium (MEM, Sigma, St. Louis, Mo.); Dulbecco's Modified Eagles Medium (DMEM, Sigma); Ham's F10 Medium (Sigma); HyClone cell culture medium (HyClone, Logan, Utah); RPMI-1640 Medium (Sigma); and chemically-defined (CD) media, which are formulated for various cell types, e.g., CD-CHO Medium (Invitrogen, Carlsbad, Calif.).

The cell culture media can be supplemented as necessary with supplementary components or ingredients, including optional components, in appropriate concentrations or amounts, as necessary or desired. Cell culture medium solutions provide at least one component from one or more of the following categories: (1) an energy source, usually in the form of a carbohydrate such as glucose; (2) all essential amino acids, and usually the basic set of twenty amino acids plus cysteine; (3) vitamins and/or other organic compounds required at low concentrations; (4) free fatty acids or lipids, for example linoleic acid; and (5) trace elements, where trace elements are defined as inorganic compounds or naturally occurring elements that can be required at very low concentrations, usually in the micromolar range.

The medium also can be supplemented electively with one or more components from any of the following categories: (1) salts, for example, magnesium, calcium, and phosphate; (2) hormones and other growth factors such as, serum, insulin, transferrin, and epidermal growth factor; (3) protein and tissue hydrolysates, for example peptone or peptone mixtures which can be obtained from purified gelatin, plant material, or animal byproducts; (4) nucleosides and bases such as, adenosine, thymidine, and hypoxanthine; (5) buffers, such as HEPES; (6) antibiotics, such as gentamycin or ampicillin; (7) cell protective agents, for example pluronic polyol; and (8) galactose. In one embodiment, soluble factors can be added to the culturing medium.

The mammalian cell culture that can be used with the present invention is prepared in a medium suitable for the type of cell being cultured. In one embodiment, the cell culture medium can be any one of those previously discussed (for example, MEM) that is supplemented with serum from a mammalian source (for example, fetal bovine serum (FBS)). In another embodiment, the medium can be a conditioned medium to sustain the growth of host cells.

Three-dimensional cultures can be formed from agar (such as Gey's Agar), hydrogels (such as matrigel, agarose, and the like; Lee et al., (2004) Biomaterials 25: 2461-2466) or polymers that are cross-linked. These polymers can comprise natural polymers and their derivatives, synthetic polymers and their derivatives, or a combination thereof. Natural polymers can be anionic polymers, cationic polymers, amphipathic polymers, or neutral polymers. Non-limiting examples of anionic polymers can include hyaluronic acid, alginic acid (alginate), carageenan, chondroitin sulfate, dextran sulfate, and pectin. Some examples of cationic polymers, include but are not limited to, chitosan or polylysine. (Peppas et al., (2006) Adv Mater. 18: 1345-60; Hoffman, A. S., (2002) Adv Drug Deliv Rev. 43: 3-12; Hoffman, A. S., (2001) Ann NY Acad Sci 944: 62-73). Examples of amphipathic polymers can include, but are not limited to collagen, gelatin, fibrin, and carboxymethyl chitin. Non-limiting examples of neutral polymers can include dextran, agarose, or pullulan. (Peppas et al., (2006) Adv Mater. 18: 1345-60; Hoffman, A. S., (2002) Adv Drug Deliv Rev. 43: 3-12; Hoffman, A. S., (2001) Ann NY Acad Sci 944: 62-73).

Cells to be cultured can harbor introduced expression vectors, such as plasmids. The expression vector constructs can be introduced via transformation, microinjection, transfection, lipofection, electroporation, or infection. The expression vectors can contain coding sequences, or portions thereof, encoding the proteins for expression and production. Expression vectors containing sequences encoding the produced proteins and polypeptides, as well as the appropriate transcriptional and translational control elements, can be generated using methods well known to and practiced by those skilled in the art. These methods include synthetic techniques, in vitro recombinant DNA techniques, and in vivo genetic recombination which are described in J. Sambrook et al., 2001, Molecular Cloning, A Laboratory Manual, Cold Spring Harbor Press, Plainview, N.Y. and in F. M. Ausubel et al., 1989, Current Protocols in Molecular Biology, John Wiley & Sons, New York, N.Y.

### FGFR Fusion Molecule Inhibitors

The invention provides methods for use of compounds that decrease the expression level or activity of a FGFR fusion molecule in a subject. In addition, the invention provides methods for using compounds for the treatment of a gene-fusion associated cancer. In one embodiment, the gene-fusion associated cancer is an epithelial cancer. In one embodiment, the gene-fusion associated cancer comprises glioblastoma multiforme, breast cancer, lung cancer, prostate cancer, or colorectal carcinoma.

As used herein, a “FGFR fusion molecule inhibitor” refers to a compound that interacts with a FGFR fusion molecule of the invention and modulates its activity and/or its expression. For example, the compound can decrease the activity or expression of a FGFR fusion molecule. The compound can be an antagonist of a FGFR fusion molecule (e.g., a FGFR fusion molecule inhibitor). Some non-limiting examples of FGFR fusion molecule inhibitors include peptides (such as peptide fragments comprising a FGFR fusion molecule, or antibodies or fragments thereof), small molecules, and nucleic acids (such as siRNA or antisense RNA specific for a nucleic acid comprising a FGFR fusion molecule). Antagonists of a FGFR fusion molecule decrease the amount or the duration of the activity of an FGFR fusion protein. In one embodiment, the fusion protein comprises a tyrosine kinase domain of an FGFR protein fused to a polypeptide that constitutively activates the tyrosine kinase domain of the FGFR protein (e.g., FGFR1-TACC1, FGFR2-TACC2, FGFR3-TACC3 or other FGFR-TACC), or a fusion protein comprises a transforming acidic coiled-coil (TACC) domain fused to a polypeptide with a tyrosine kinase domain, wherein the TACC domain constitutively activates the tyrosine kinase domain. Antagonists include proteins, nucleic acids, antibodies, small molecules, or any other molecule which decrease the activity of a FGFR fusion molecule.

The term “modulate,” as it appears herein, refers to a change in the activity or expression of a FGFR fusion molecule. For example, modulation can cause a decrease in protein activity, binding characteristics, or any other biological, functional, or immunological properties of a FGFR fusion molecule, such as an FGFR fusion protein.

In one embodiment, a FGFR fusion molecule inhibitor can be a peptide fragment of a FGFR fusion protein that binds to the protein itself.

For example, the FGFR fusion polypeptide can encompass any portion of at least about 8 consecutive amino acids of SEQ ID NOS: 79, 88, 150, 158-161, or 539-547. The fragment can comprise at least about 10 consecutive amino acids, at least about 20 consecutive amino acids, at least about 30 consecutive amino acids, at least about 40 consecutive amino acids, a least about 50 consecutive amino acids, at least about 60 consecutive amino acids, at least about 70 consecutive amino acids, at least about 75 consecutive amino acids, at least about 80 consecutive amino acids, at least about 85 consecutive amino acids, at least about 90 consecutive amino acids, at least about 95 consecutive amino acids, at least about 100 consecutive amino acids, at least about 200 consecutive amino acids, at least about 300 consecutive amino acids, at least about 400 consecutive amino acids, at least about 500 consecutive amino acids, at least about 600 consecutive amino acids, at least about 700 consecutive amino acids, or at least about 800 consecutive amino acids of SEQ ID NOS: 79, 88, 150, 158-161, or 539-547. Fragments include all possible amino acid lengths between about 8 and 100 about amino acids, for example, lengths between about 10 and about 100 amino acids, between about 15 and about 100 amino acids, between about 20 and about 100 amino acids, between about 35 and about 100 amino acids, between about 40 and about 100 amino acids, between about 50 and about 100 amino acids, between about 70 and about 100 amino acids, between about 75 and about 100 amino acids, or between about 80 and about 100 amino acids. These peptide fragments can be obtained commercially or synthesized via liquid phase or solid phase synthesis methods (Atherton et al., (1989) Solid Phase Peptide Synthesis: a Practical Approach. IRL Press, Oxford, England). The FGFR fusion peptide fragments can be isolated from a natural source, genetically engineered, or chemically prepared. These methods are well known in the art.

A FGFR fusion molecule inhibitor can be a protein, such as an antibody (monoclonal, polyclonal, humanized, chimeric, or fully human), or a binding fragment thereof, directed against a FGFR fusion molecule of the invention. An antibody fragment can be a form of an antibody other than the full-length form and includes portions or components that exist within full-length antibodies, in addition to antibody fragments that have been engineered. Antibody fragments can include, but are not limited to, single chain Fv (scFv), diabodies, Fv, and (Fab′)₂, triabodies, Fc, Fab, CDR1, CDR2, CDR3, combinations of CDR's, variable regions, tetrabodies, bifunctional hybrid antibodies, framework regions, constant regions, and the like (see, Maynard et al., (2000) Ann. Rev. Biomed. Eng. 2:339-76; Hudson (1998) Curr. Opin. Biotechnol. 9:395-402). Antibodies can be obtained commercially, custom generated, or synthesized against an antigen of interest according to methods established in the art (see U.S. Pat. Nos. 6,914,128, 5,780,597, 5,811,523; Roland E. Kontermann and Stefan Diibel (editors), Antibody Engineering, Vol. I & II, (2010) 2^{nd} ed., Springer; Antony S. Dimitrov (editor), Therapeutic Antibodies: Methods and Protocols (Methods in Molecular Biology), (2009), Humana Press; Benny Lo (editor) Antibody Engineering: Methods and Protocols (Methods in Molecular Biology), (2004) Humana Press, each of which are hereby incorporated by reference in their entireties). For example, antibodies directed to a FGFR fusion molecule can be obtained commercially from Abcam, Santa Cruz Biotechnology, Abgent, R&D Systems, Novus Biologicals, etc. Human antibodies directed to a FGFR fusion molecule (such as monoclonal, humanized, fully human, or chimeric antibodies) can be useful antibody therapeutics for use in humans. In one embodiment, an antibody or binding fragment thereof is directed against SEQ ID NOS: 79, 88, 150, 158-161, or 539-547.

Inhibition of RNA encoding a FGFR fusion molecule can effectively modulate the expression of a FGFR fusion molecule. Inhibitors are selected from the group comprising: siRNA; interfering RNA or RNAi; dsRNA; RNA Polymerase III transcribed DNAs; ribozymes; and antisense nucleic acids, which can be RNA, DNA, or an artificial nucleic acid.

Antisense oligonucleotides, including antisense DNA, RNA, and DNA/RNA molecules, act to directly block the translation of mRNA by binding to targeted mRNA and preventing protein translation. For example, antisense oligonucleotides of at least about 15 bases and complementary to unique regions of the DNA sequence encoding a FGFR fusion molecule can be synthesized, e.g., by conventional phosphodiester techniques (Dallas et al., (2006) Med. Sci. Monit. 12(4):RA67-74; Kalota et al., (2006) Handb. Exp. Pharmacol. 173:173-96; Lutzelburger et al., (2006) Handb. Exp. Pharmacol. 173:243-59). Antisense nucleotide sequences include, but are not limited to: morpholinos, 2′-O-methyl polynucleotides, DNA, RNA and the like.

siRNA comprises a double stranded structure containing from about 15 to about 50 base pairs, for example from about 21 to about 25 base pairs, and having a nucleotide sequence identical or nearly identical to an expressed target gene or RNA within the cell. The siRNA comprise a sense RNA strand and a complementary antisense RNA strand annealed together by standard Watson-Crick base-pairing interactions. The sense strand comprises a nucleic acid sequence which is substantially identical to a nucleic acid sequence contained within the target miRNA molecule. “Substantially identical” to a target sequence contained within the target mRNA refers to a nucleic acid sequence that differs from the target sequence by about 3% or less. The sense and antisense strands of the siRNA can comprise two complementary, single-stranded RNA molecules, or can comprise a single molecule in which two complementary portions are base-paired and are covalently linked by a single-stranded “hairpin” area. See also, McMnaus and Sharp (2002) Nat Rev Genetics, 3:737-47, and Sen and Blau (2006) FASEB J., 20:1293-99, the entire disclosures of which are herein incorporated by reference.

The siRNA can be altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the siRNA, or modifications that make the siRNA resistant to nuclease digestion, or the substitution of one or more nucleotides in the siRNA with deoxyribo-nucleotides. One or both strands of the siRNA can also comprise a 3′ overhang. As used herein, a 3′ overhang refers to at least one unpaired nucleotide extending from the 3′-end of a duplexed RNA strand. For example, the siRNA can comprise at least one 3′ overhang of from 1 to about 6 nucleotides (which includes ribonucleotides or deoxyribonucleotides) in length, or from 1 to about 5 nucleotides in length, or from 1 to about 4 nucleotides in length, or from about 2 to about 4 nucleotides in length. For example, each strand of the siRNA can comprise 3′ overhangs of dithymidylic acid (“TT”) or diuridylic acid (“uu”).

siRNA can be produced chemically or biologically, or can be expressed from a recombinant plasmid or viral vector (for example, see U.S. Pat. Nos. 7,294,504 and 7,422,896, the entire disclosures of which are herein incorporated by reference). Exemplary methods for producing and testing dsRNA or siRNA molecules are described in U.S. Patent Application Publication No. 2002/0173478 to Gewirtz, U.S. Pat. No. 8,071,559 to Hannon et al., and in U.S. Pat. No. 7,148,342 to Tolentino et al., the entire disclosures of which are herein incorporated by reference.

In one embodiment, an siRNA directed to a human nucleic acid sequence comprising a FGFR fusion molecule can be generated against any one of SEQ ID NOS: 80-82, 84, 94-145, 515, 517, 519-527 or 530-538. In another embodiment, an siRNA directed to a human nucleic acid sequence comprising a breakpoint of an FGFR fusion molecule can be generated against any one of SEQ ID NOS: 1-77, 80-82, 84-145, 515, 517, 519-527 or 530-538. In one embodiment, the hairpin sequences targeting the FGFR3 gene comprise SEQ ID NOS: 182, 183, or 184.

RNA polymerase III transcribed DNAs contain promoters, such as the U6 promoter. These DNAs can be transcribed to produce small hairpin RNAs in the cell that can function as siRNA or linear RNAs, which can function as antisense RNA. The FGFR fusion molecule inhibitor can comprise ribonucleotides, deoxyribonucleotides, synthetic nucleotides, or any suitable combination such that the target RNA and/or gene is inhibited. In addition, these forms of nucleic acid can be single, double, triple, or quadruple stranded. (see for example Bass (2001) Nature, 411:428-429; Elbashir et al., (2001) Nature, 411:494 498; U.S. Pat. No. 6,509,154; U.S. Patent Application Publication No. 2003/0027783; and PCT Publication Nos. WO 00/044895, WO 99/032619, WO 00/01846, WO 01/029058, WO 00/044914).

FGFR fusion molecule inhibitor can be a small molecule that binds to a FGFR fusion protein described herein and disrupts its function. Small molecules are a diverse group of synthetic and natural substances generally having low molecular weights. They can be isolated from natural sources (for example, plants, fungi, microbes and the like), are obtained commercially and/or available as libraries or collections, or synthesized. Candidate small molecules that inhibit a FGFR fusion protein can be identified via in silico screening or high-through-put (HTP) screening of combinatorial libraries according to methods established in the art (e.g., see Potyrailo et al., (2011) ACS Comb Sci. 13(6):579-633; Mensch et al., (2009) J Pharm Sci. 98(12):4429-68; Schnur (2008) Curr Opin Drug Discov Devel. 11(3):375-80; and Jhoti (2007) Ernst Schering Found Symp Proc. (3):169-85, each of which are hereby incorporated by reference in their entireties.) Most conventional pharmaceuticals, such as aspirin, penicillin, and many chemotherapeutics, are small molecules, can be obtained commercially, can be chemically synthesized, or can be obtained from random or combinatorial libraries as described below (see, e.g., Werner et al., (2006) Brief Funct. Genomic Proteomic 5(1):32-6).

Non-limiting examples of FGFR fusion molecule inhibitors include the FGFR inhibitors AZD4547 (see Gavine et al., (2012) Cancer Res, 72(8); 2045-56; see also PCT Application Publication No. WO 2008/075068, each of which are hereby incorporated by reference in their entireties); NVP-BGJ398 (see Guagnano et al., (2011) J. Med. Chem., 54:7066-7083; see also U.S. Patent Application Publication No. 2008-0312248 A1, each of which are hereby incorporated by reference in their entireties); PD173074 (see Guagnano et al., (2011) J. Med. Chem., 54:7066-7083; see also Mohammadi et al., (1998) EMBO J., 17:5896-5904, each of which are hereby incorporated by reference in their entireties); NF449 (EMD Millipore (Billerica, MA) Cat. No. 480420; see also Krejci, (2010) the Journal of Biological Chemistry, 285(27):20644-20653, which is hereby incorporated by reference in its entirety); LY2874455 (Active Biochem; see Zhao et al. (2011) Mol Cancer Ther. (11):2200-10; see also PCT Application Publication No. WO 2010129509, each of which are hereby incorporated by reference in their entireties); TK1258 (Dovitinib); BIBF-1120 (Intedanib-Vargatef); BMS-582664 (Brivanib alaninate); AZD-2171 (Cediranib); TSU-68 (Orantinib); AB-1010 (Masitinib); AP-24534 (Ponatinib); and E-7080 (by Eisai). A non-limiting example of an FGFR fusion molecule inhibitor includes the TACC inhibitor KHS101 (Wurdak et al., (2010) PNAS, 107(38): 16542-47, which is hereby incorporated by reference in its entirety).

Structures of FGFR fusion molecule inhibitors useful for the invention include, but are not limited to: the FGFR inhibitor AZD4547,

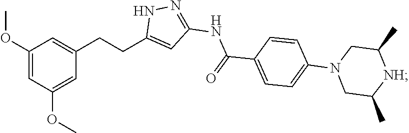 [SMILES: COc1cc(CCc2cc(NC(=O)c3ccc(N4C[C@@H](C)N[C@@H](C)C4)cc3)n[nH]2)cc(OC)c1]

the FGFR inhibitor NVP-BGJ398,

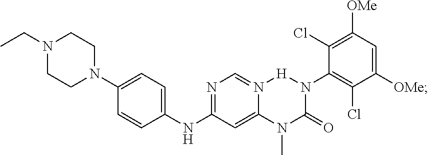

the FGFR inhibitor PD173074,

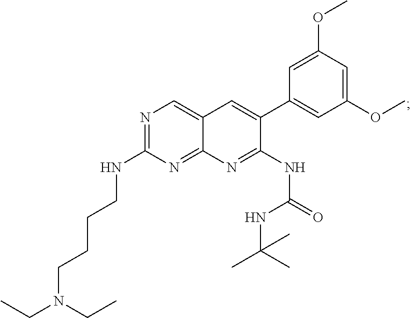 [SMILES: CCN(CC)CCCCNc1ncc2cc(-c3cc(OC)cc(OC)c3)c(NC(=O)NC(C)(C)C)nc2n1]

the FGFR inhibitor LY2874455

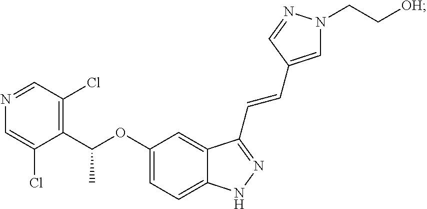 [SMILES: C[C@@H](Oc1ccc2[nH]nc(/C=C/c3cnn(CCO)c3)c2c1)c1c(Cl)cncc1Cl]

and the FGFR inhibitor NF449 (EMD Millipore (Billerica, MA) Cat. No. 480420),

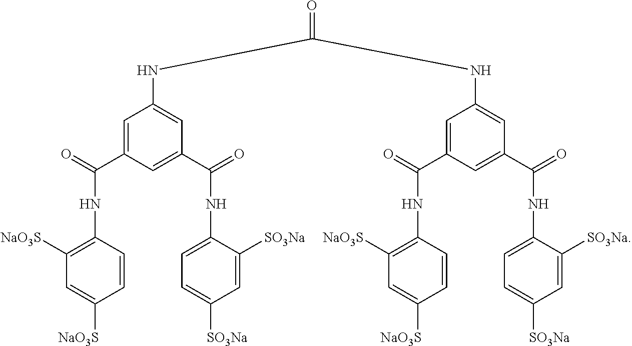

Other FGFR inhibitors include, but are not limited to:

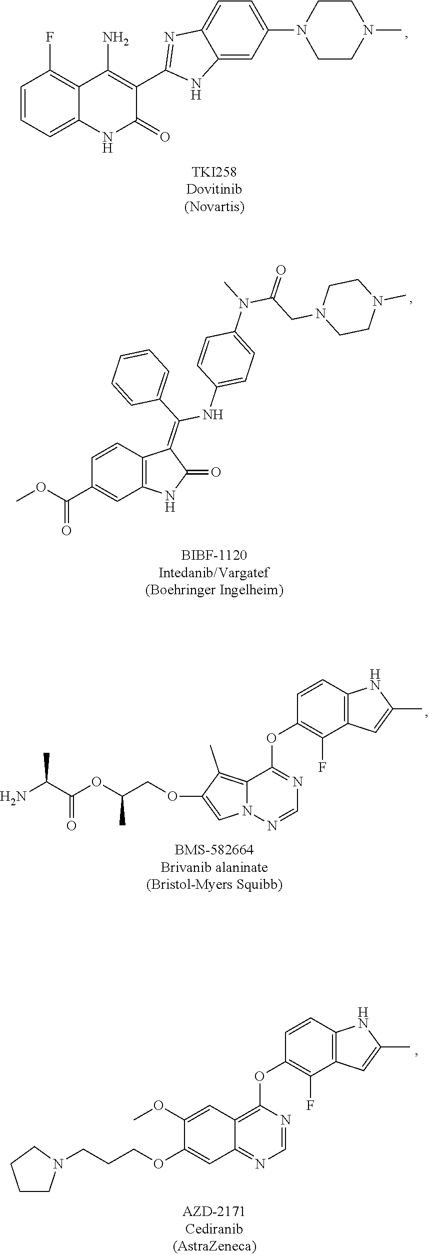

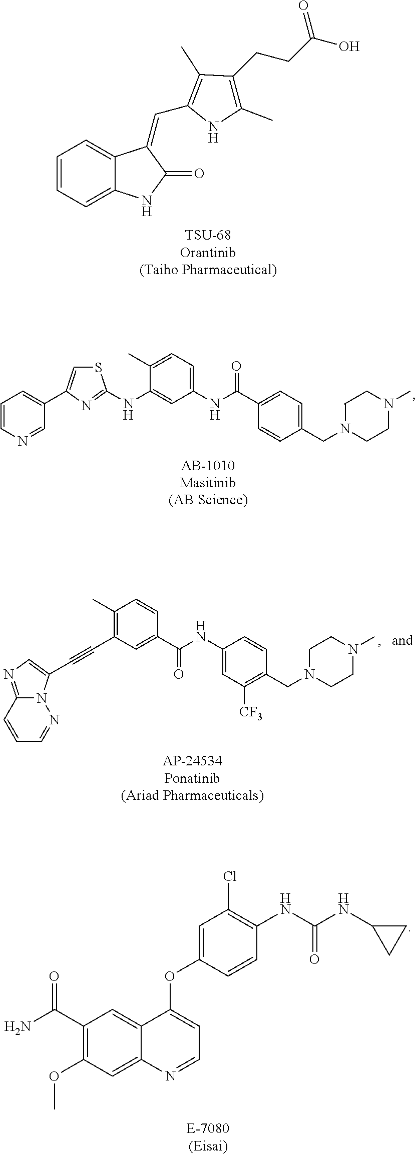 [SMILES: Cc1[nH]c(/C=C2\C(=O)Nc3ccccc32)c(C)c1CCC(=O)O ; Cc1ccc(NC(=O)c2ccc(CN3CCN(C)CC3)cc2)cc1Nc1nc(-c2cccnc2)cs1 ; Cc1ccc(C(=O)Nc2ccc(CN3CCN(C)CC3)c(C(F)(F)F)c2)cc1C#Cc1cnc2cccnn12 ; COc1cc2nccc(Oc3ccc(NC(=O)NC4CC4)c(Cl)c3)c2cc1C(N)=O]

In other embodiments, the FGFR fusion molecule inhibitor comprises an oral pan-FGFR tyrosine kinase inhibitor. In other embodiments, the FGFR fusion molecule inhibitor comprises JNJ-42756493. Structures of FGFR fusion molecule inhibitors useful for the invention include, but are not limited to: the FGFR inhibitor JNJ-42′756493.

A structure of an FGFR fusion molecule inhibitor useful for the invention include, but is not limited to the TACC inhibitor KHS101,

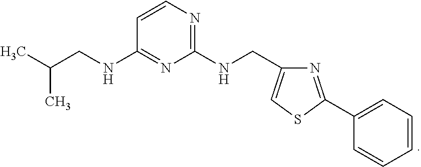 [SMILES: CC(C)CNc1ccnc(NCc2csc(-c3ccccc3)n2)n1]

### Assessment and Therapeutic Treatment

The invention provides a method of decreasing the growth of a solid tumor in a subject. The tumor is associated with, but not limited to, glioblastoma multiforme, breast cancer, lung cancer, prostate cancer, or colorectal carcinoma. In another embodiment, the tumor is associated with, but not limited to, bladder carcinoma, squamous lung carcinoma and head and neck carcinoma. In one embodiment, the tumor is associated with, but not limited to, glioma. In one embodiment, the tumor is associated with, but not limited to, grade II or III glioma. In one embodiment, the tumor is associated with, but not limited to, IDH wild-type grade II or III glioma. In one embodiment, the method comprises detecting the presence of a FGFR fusion molecule in a sample obtained from a subject. In some embodiments, the sample is incubated with an agent that binds to an FGFR fusion molecule, such as an antibody, a probe, a nucleic acid primer, and the like. In further embodiments, the method comprises administering to the subject an effective amount of a FGFR fusion molecule inhibitor, wherein the inhibitor decreases the size of the solid tumor. In further embodiments, the method comprises further detecting the presence of IDH1 mutations, EGFR amplification, CDK4 amplification, or MDM2 amplification. In further embodiments, a FGFR fusion molecule inhibitor can be administered in combination with CDK4 inhibitors, MDM2 inhibitors, or a combination thereof.

The invention also provides a method for treating or preventing a gene-fusion associated cancer in a subject. In one embodiment, the gene-fusion associated cancer comprises an epithelial cancer. In one embodiment, the gene-fusion associated cancer comprises glioblastoma multiforme, breast cancer, lung cancer, prostate cancer, or colorectal carcinoma. In some embodiments, the epithelial cancer comprises bladder urothelial carcinoma, breast carcinoma, colorectal cancer, prostate carcinoma, lung squamous cell carcinoma, head and neck squamous cell carcinoma, or a combination of the epithelial cancers described. In one embodiment, the gene-fusion associated cancer comprises glioma. In one embodiment, the gene-fusion associated cancer comprises grade II or III glioma. In one embodiment, the gene-fusion associated cancer comprises IDH wild-type grade II or III glioma. In one embodiment, the method comprises detecting the presence of a FGFR fusion molecule in a sample obtained from a subject, the presence of the fusion being indicative of a gene-fusion associated cancer, and, administering to the subject in need a therapeutic treatment against a gene-fusion associated cancer. In some embodiments, the sample is incubated with an agent that binds to an FGFR fusion molecule, such as an antibody, a probe, a nucleic acid primer, and the like. In further embodiments, the method comprises further detecting the presence of IDH1 mutations, EGFR amplification, CDK4 amplification, or MDM2 amplification. In further embodiments, an agent that binds to an FGFR fusion molecule can be administered in combination with CDK4 inhibitors, MDM2 inhibitors, or a combination thereof.

The invention also provides a method for decreasing in a subject in need thereof the expression level or activity of a fusion protein comprising the tyrosine kinase domain of an FGFR protein fused to a polypeptide that constitutively activates the tyrosine kinase domain of the FGFR protein. In some embodiments, the method comprises obtaining a biological sample from the subject. In some embodiments, the sample is incubated with an agent that binds to an FGFR fusion molecule, such as an antibody, a probe, a nucleic acid primer, and the like. In some embodiments, the method comprises administering to the subject a therapeutic amount of a composition comprising an admixture of a pharmaceutically acceptable carrier an inhibitor of the fusion protein of the invention. In one embodiment, the inhibitor is JNJ-42756493. In another embodiment, the method further comprises determining the fusion protein expression level or activity. In another embodiment, the method further comprises detecting whether the fusion protein expression level or activity is decreased as compared to the fusion protein expression level or activity prior to administration of the composition, thereby decreasing the expression level or activity of the fusion protein. In some embodiments, the fusion protein is an FGFR-TACC fusion protein. In further embodiments, the method comprises further detecting the presence of IDH1 mutations, EGFR amplification, CDK4 amplification, or MDM2 amplification.

The administering step in each of the claimed methods can comprise a drug administration, such as FGFR fusion molecule inhibitor (for example, a pharmaceutical composition comprising an antibody that specifically binds to a FGFR fusion molecule or a fragment thereof; a small molecule that specifically binds to a FGFR protein; a small molecule that specifically binds to a TACC protein; an antisense RNA or antisense DNA that decreases expression of a FGFR fusion molecule; a siRNA that specifically targets a gene encoding a FGFR fusion molecule; a small molecule such as JNJ-42756493; or a combination thereof). In one embodiment, the therapeutic molecule to be administered comprises a polypeptide of a FGFR fusion molecule, comprising at least about 75%, at least about 80%, at least about 85%, at least about 90%, at least about 93%, at least about 95%, at least about 97%, at least about 98%, at least about 99%, or 100% of the amino acid sequence of SEQ ID NOS: 79, 88, 150, 158-161, or 539-547 and exhibits the function of decreasing expression of such a protein, thus treating a gene fusion-associated cancer. In another embodiment, administration of the therapeutic molecule decreases the size of the solid tumor associated with glioblastoma multiforme, breast cancer, lung cancer, prostate cancer, colorectal carcinoma, bladder carcinoma, squamous lung carcinoma and head and neck carcinoma, glioma, grade II or III glioma, or IDH wild-type grade II or III glioma. In further embodiments, the therapeutic molecule can be administered in combination with CDK4 inhibitors, MDM2 inhibitors, or a combination thereof.

In another embodiment, the therapeutic molecule to be administered comprises an siRNA directed to a human nucleic acid sequence comprising a FGFR fusion molecule. In one embodiment, the siRNA is directed to any one of SEQ ID NOS: 80-82, 84, 94-145, 515, 517, 519-527, or 530-538. In another embodiment, the siRNA is directed to any one of SEQ ID NOS: 1-77, 80-82, 84-145, 515, 517, 519-527, or 530-538. In a further embodiment, the therapeutic molecule to be administered comprises an antibody or binding fragment thereof, which is directed against SEQ ID NOS: 79, 88, 150, 158-161, or 539-547. In some embodiments, the therapeutic molecule to be administered comprises a small molecule that specifically binds to a FGFR protein, such as AZD4547, NVP-BGJ398, PD173074, NF449, TK1258, BIBF-1120, BMS-582664, AZD-2171, TSU68, AB1010, AP24534, E-7080, or LY2874455. In some embodiments, the therapeutic molecule to be administered is JNJ-42756493. In other embodiments, the therapeutic molecule to be administered comprises a small molecule that specifically binds to a TACC protein, such as KHS101.

In one embodiment, the invention provides for the detection of a chromosomal rearrangement at given chromosomal coordinates. In another embodiment, the detection or determination comprises nucleic acid sequencing, selective hybridization, selective amplification, gene expression analysis, or a combination thereof. In another embodiment, the detection or determination comprises protein expression analysis, for example by western blot analysis, immunostaining, ELISA, or other antibody detection methods.

In one embodiment, the biological sample comprises neuronal cells, serum, bone marrow, blood, peripheral blood, lymph nodes, cerebro-spinal fluid, urine, a saliva sample, a buccal swab, a serum sample, a sputum sample, a lacrimal secretion sample, a semen sample, a vaginal secretion sample, a fetal tissue sample, or a combination thereof. In some embodiments the sample is a tissue sample. In some embodiments, the sample is a paraffin embedded tissue section. In some embodiments, the tissue sample is a tumor sample.

A FGFR fusion molecule, for example, a fusion between FGFR1, FGFR2, FGFR3, or any other FGFR, and TACC1, TACC2, TACC3 or any other TACC, can be determined at the level of the DNA, RNA, or polypeptide. Optionally, detection can be determined by performing an oligonucleotide ligation assay, a confirmation based assay, a hybridization assay, a sequencing assay, an allele-specific amplification assay, a microsequencing assay, a melting curve analysis, a denaturing high performance liquid chromatography (DHPLC) assay (for example, see Jones et al, (2000) Hum Genet., 106(6):663-8), or a combination thereof. In one embodiment, the detection is performed by sequencing all or part of a FGFR fusion molecule (e.g., a FGFR1-TACC1, FGFR2-TACC2, FGFR3-TACC3 or other FGFR-TACC nucleic acid, or a FGFR1, TACC1, FGFR2, TACC2, FGFR3, TACC3 or other FGFR or TACC nucleic acid), or by selective hybridization or amplification of all or part of a FGFR fusion molecule (e.g., a FGFR1-TACC1, FGFR2-TACC2, FGFR3-TACC3 or other FGFR-TACC nucleic acid, or a FGFR1, TACC1, FGFR2, TACC2, FGFR3, TACC3 or other FGFR or TACC nucleic acid). A FGFR fusion molecule specific amplification (e.g., a FGFR1-TACC1, FGFR2-TACC2, FGFR3-TACC3 or other FGFR-TACC nucleic acid specific amplification) can be carried out before the fusion identification step.

The invention provides for a method of detecting a chromosomal alteration in a subject afflicted with a gene-fusion associated cancer. In one embodiment, the chromosomal alteration is an in-frame fused transcript described herein, for example an FGFR fusion molecule. In some embodiments, the chromosomal alteration is a chromosomal translocation, for example an FGFR fusion molecule. An alteration in a chromosome region occupied by a FGFR fusion molecule, such as a FGFR1-TACC1, FGFR2-TACC2, FGFR3-TACC3 or other FGFR-TACC nucleic acid, can be any form of mutation(s), deletion(s), rearrangement(s) and/or insertions in the coding and/or non-coding region of the locus, alone or in various combination(s). Mutations can include point mutations. Insertions can encompass the addition of one or several residues in a coding or non-coding portion of the gene locus. Insertions can comprise an addition of between 1 and 50 base pairs in the gene locus. Deletions can encompass any region of one, two or more residues in a coding or non-coding portion of the gene locus, such as from two residues up to the entire gene or locus. Deletions can affect smaller regions, such as domains (introns) or repeated sequences or fragments of less than about 50 consecutive base pairs, although larger deletions can occur as well. Rearrangement includes inversion of sequences. The alteration in a chromosome region occupied by a FGFR fusion molecule, e.g., a FGFR1-TACC1, FGFR2-TACC2, FGFR3-TACC3 or other FGFR-TACC nucleic acid, can result in amino acid substitutions, RNA splicing or processing, product instability, the creation of stop codons, production of oncogenic fusion proteins, frame-shift mutations, and/or truncated polypeptide production. The alteration can result in the production of a FGFR fusion molecule, for example, one encoded by a FGFR1-TACC1, FGFR2-TACC2, FGFR3-TACC3 or other FGFR-TACC nucleic acid, with altered function, stability, targeting or structure. The alteration can also cause a reduction, or even an increase in protein expression. In one embodiment, the alteration in the chromosome region occupied by a FGFR fusion molecule can comprise a chromosomal rearrangement resulting in the production of a FGFR fusion molecule, such as a FGFR1-TACC1, FGFR2-TACC2, FGFR3-TACC3 or other FGFR-TACC fusion. This alteration can be determined at the level of the DNA, RNA, or polypeptide. In another embodiment, the detection or determination comprises nucleic acid sequencing, selective hybridization, selective amplification, gene expression analysis, or a combination thereof. In another embodiment, the detection or determination comprises protein expression analysis, for example by western blot analysis, ELISA, immunostaining or other antibody detection methods. In one embodiment, the coordinates comprising FGFR1 translocations comprise chr8:38,268,656-38,325,363. In another embodiment, the coordinates comprising FGFR2 translocations comprise chr10:123,237,844-123,357,972. In a further embodiment, the coordinates comprising FGFR3 translocations comprise chr4:1,795,039-1,810,599. In yet another embodiment, the coordinates comprising FGFR4 translocations comprise chr5:176,513,921-176,525,126. In one embodiment, the coordinates comprising TACC1 translocations comprise chr8:38,644,722-38,710,546. In another embodiment, the coordinates comprising TACC2 translocations comprise chr10:123,748,689-124,014,057. In a further embodiment, the coordinates comprising TACC3 translocations comprise chr4:1,723,217-1,746,905.

The present invention provides a method for treating a gene-fusion associated cancer in a subject in need thereof. In one embodiment, the method comprises obtaining a sample from the subject to determine the level of expression of an FGFR fusion molecule in the subject. In some embodiments, the sample is incubated with an agent that binds to an FGFR fusion molecule, such as an antibody, a probe, a nucleic acid primer, and the like. In another embodiment, the detection or determination comprises nucleic acid sequencing, selective hybridization, selective amplification, gene expression analysis, or a combination thereof. In another embodiment, the detection or determination comprises protein expression analysis, for example by western blot analysis, immunostaining, ELISA, or other antibody detection methods. In some embodiments, the method further comprises assessing whether to administer a FGFR fusion molecule inhibitor based on the expression pattern of the subject. In further embodiments, the method comprises administering a FGFR fusion molecule inhibitor to the subject. In one embodiment, the FGFR fusion molecule inhibitor is JNJ-42756493. In one embodiment, the gene-fusion associated cancer comprises an epithelial cancer. In one embodiment, the gene-fusion associated cancer comprises glioblastoma multiforme, breast cancer, lung cancer, prostate cancer, or colorectal carcinoma. In some embodiments, the epithelial cancer comprises bladder urothelial carcinoma, breast carcinoma, colorectal cancer, prostate carcinoma, lung squamous cell carcinoma, head and neck squamous cell carcinoma, or a combination of the epithelial cancers described. In one embodiment, the gene-fusion associated cancer comprises glioma, grade II or III glioma, or IDH wild-type grade II or III glioma. In further embodiments, the method comprises further detecting the presence of IDH1 mutations, EGFR amplification, CDK4 amplification, or MDM2 amplification. In further embodiments, a FGFR fusion molecule inhibitor can be administered in combination with CDK4 inhibitors, MDM2 inhibitors, or a combination thereof.

In one embodiment, the invention provides for a method of detecting the presence of altered RNA expression of an FGFR fusion molecule in a subject, for example one afflicted with a gene-fusion associated cancer. In another embodiment, the invention provides for a method of detecting the presence of an FGFR fusion molecule in a subject. In some embodiments, the method comprises obtaining a sample from the subject to determine whether the subject expresses an FGFR fusion molecule. In some embodiments, the sample is incubated with an agent that binds to an FGFR fusion molecule, such as an antibody, a probe, a nucleic acid primer, and the like. In other embodiments, the detection or determination comprises nucleic acid sequencing, selective hybridization, selective amplification, gene expression analysis, or a combination thereof. In another embodiment, the detection or determination comprises protein expression analysis, for example by western blot analysis, ELISA, or other antibody detection methods. In some embodiments, the method further comprises assessing whether to administer a FGFR fusion molecule inhibitor based on the expression pattern of the subject. In further embodiments, the method comprises administering a FGFR fusion molecule inhibitor to the subject. In one embodiment, the FGFR fusion molecule inhibitor is JNJ-42756493. Altered RNA expression includes the presence of an altered RNA sequence, the presence of an altered RNA splicing or processing, or the presence of an altered quantity of RNA. These can be detected by various techniques known in the art, including sequencing all or part of the RNA or by selective hybridization or selective amplification of all or part of the RNA. In a further embodiment, the method can comprise detecting the presence or expression of a FGFR fusion molecule, such as one encoded by a FGFR1-TACC1, FGFR2-TACC2, FGFR3-TACC3 or other FGFR-TACC nucleic acid. Altered polypeptide expression includes the presence of an altered polypeptide sequence, the presence of an altered quantity of polypeptide, or the presence of an altered tissue distribution. These can be detected by various techniques known in the art, including by sequencing and/or binding to specific ligands (such as antibodies). In one embodiment, the detecting comprises using a northern blot; real time PCR and primers directed to SEQ ID NOS: 80-82, 84, 94-145, 515, 517, 519-527, or 530-538; a ribonuclease protection assay; a hybridization, amplification, or sequencing technique to detect an FGFR fusion molecule, such as one comprising SEQ ID NOS: 80-82, 84, 94-145, 515, 517, 519-527, or 530-538; or a combination thereof. In another embodiment, the PCR primers comprise SEQ ID NOS: 162, 163, 164, 165 166, 167, 168, 169, 495, 496, 497, 498, 507, 508, 509, 510, 511, 512, 513, or 514. In a further embodiment, primers used for the screening of FGFR fusion molecules, such as FGFR-TACC fusions, comprise SEQ ID NOS: 166, 167, 168, 169, 495, 496, 497, 498, 507, 508, 509, or 510. In some embodiments, primers used for genomic detection of an FGFR3-TACC3 fusion comprise SEQ ID NOS: 170 171, 499, 500, 501, 502, 503, 504, 505, or 506.

In some aspects of the invention, the method comprises further detecting the presence of IDH1 mutations, EGFR amplification, CDK4 amplification, or MDM2 amplification. MDM2 encodes a nuclear-localized E3 ubiquitin ligase. Alternative splicing results in a multitude of transcript variants, many of which may be expressed only in tumor cells. EGFR (epidermal growth factor receptor) is a transmembrane glycoprotein that is a member of the protein kinase superfamily. This protein is a receptor for members of the epidermal growth factor family. EGFR is a cell surface protein that binds to epidermal growth factor. Multiple alternatively spliced transcript variants that encode different protein isoforms have been found for this gene. CDK4 (cyclin dependent kinase 4) is a member of the Ser/Thr protein kinase family. It is a catalytic subunit of the protein kinase complex that is important for cell cycle G1 phase progression. The activity of this kinase is restricted to the G1-S phase, which is controlled by the regulatory subunits D-type cyclins and CDK inhibitor p16(INK4a). Multiple polyadenylation sites of this gene have been reported. IDH1 (isocitrate dehydrogenase 1 (NADP+), soluble) catalyzes the oxidative decarboxylation of isocitrate to 2-oxoglutarate. Alternatively spliced transcript variants encoding the same protein have been found for this gene. IDH1 mutations, EGFR amplification, CDK4 amplification, or MDM2 amplification can be detected using various techniques know in the art, including, but not limited to sequencing and qPCR.

Various techniques known in the art can be used to detect or quantify altered gene or RNA expression or nucleic acid sequences, which include, but are not limited to, hybridization, sequencing, amplification, and/or binding to specific ligands (such as antibodies). Other suitable methods include allele-specific oligonucleotide (ASO), oligonucleotide ligation, allele-specific amplification, Southern blot (for DNAs), Northern blot (for RNAs), single-stranded conformation analysis (SSCA), PFGE, fluorescent in situ hybridization (FISH), gel migration, clamped denaturing gel electrophoresis, denaturing HLPC, melting curve analysis, heteroduplex analysis, RNase protection, chemical or enzymatic mismatch cleavage, ELISA, radio-immunoassays (RIA) and immuno-enzymatic assays (IEMA).

Some of these approaches (such as SSCA and constant gradient gel electrophoresis (CGGE)) are based on a change in electrophoretic mobility of the nucleic acids, as a result of the presence of an altered sequence. According to these techniques, the altered sequence is visualized by a shift in mobility on gels. The fragments can then be sequenced to confirm the alteration. Some other approaches are based on specific hybridization between nucleic acids from the subject and a probe specific for wild type or altered gene or RNA. The probe can be in suspension or immobilized on a substrate. The probe can be labeled to facilitate detection of hybrids. Some of these approaches are suited for assessing a polypeptide sequence or expression level, such as Northern blot, ELISA and RIA. These latter require the use of a ligand specific for the polypeptide, for example, the use of a specific antibody.

Hybridization. Hybridization detection methods are based on the formation of specific hybrids between complementary nucleic acid sequences that serve to detect nucleic acid sequence alteration(s). A detection technique involves the use of a nucleic acid probe specific for a wild type or altered gene or RNA, followed by the detection of the presence of a hybrid. The probe can be in suspension or immobilized on a substrate or support (for example, as in nucleic acid array or chips technologies). The probe can be labeled to facilitate detection of hybrids. In one embodiment, the probe according to the invention can comprise a nucleic acid directed to SEQ ID NOS: 80-82, 84, 94-145, 515, 517, 519-527, or 530-538. For example, a sample from the subject can be contacted with a nucleic acid probe specific for a gene encoding a FGFR fusion molecule, and the formation of a hybrid can be subsequently assessed. In one embodiment, the method comprises contacting simultaneously the sample with a set of probes that are specific for an FGFR fusion molecule. Also, various samples from various subjects can be investigated in parallel.

According to the invention, a probe can be a polynucleotide sequence which is complementary to and specifically hybridizes with a, or a target portion of a, gene or RNA corresponding to a FGFR fusion molecule. Useful probes are those that are complementary to the gene, RNA, or target portion thereof. Probes can comprise single-stranded nucleic acids of between 8 to 1000 nucleotides in length, for instance between 10 and 800, between 15 and 700, or between 20 and 500. Longer probes can be used as well. A useful probe of the invention is a single stranded nucleic acid molecule of between 8 to 500 nucleotides in length, which can specifically hybridize to a region of a gene or RNA that corresponds to a FGFR fusion molecule.

The sequence of the probes can be derived from the sequences of the FGFR fusion genes provided herein. Nucleotide substitutions can be performed, as well as chemical modifications of the probe. Such chemical modifications can be accomplished to increase the stability of hybrids (e.g., intercalating groups) or to label the probe. Some examples of labels include, without limitation, radioactivity, fluorescence, luminescence, and enzymatic labeling.

A guide to the hybridization of nucleic acids is found in e.g., Sambrook, ed., Molecular Cloning: A Laboratory Manual (3^{rd} Ed.), Vols. 1-3, Cold Spring Harbor Laboratory, 1989; Current Protocols In Molecular Biology, Ausubel, ed. John Wiley & Sons, Inc., New York, 2001; Laboratory Techniques In Biochemistry And Molecular Biology: Hybridization With Nucleic Acid Probes, Part I. Theory and Nucleic Acid Preparation, Tijssen, ed. Elsevier, N.Y., 1993.

Sequencing. Sequencing can be carried out using techniques well known in the art, using automatic sequencers. The sequencing can be performed on the complete FGFR fusion molecule or on specific domains thereof.

Amplification. Amplification is based on the formation of specific hybrids between complementary nucleic acid sequences that serve to initiate nucleic acid reproduction. Amplification can be performed according to various techniques known in the art, such as by polymerase chain reaction (PCR), ligase chain reaction (LCR), strand displacement amplification (SDA) and nucleic acid sequence based amplification (NASBA). These techniques can be performed using commercially available reagents and protocols. Useful techniques in the art encompass real-time PCR, allele-specific PCR, or PCR based single-strand conformational polymorphism (SSCP). Amplification usually requires the use of specific nucleic acid primers, to initiate the reaction. For example, nucleic acid primers useful for amplifying sequences corresponding to a FGFR fusion molecule are able to specifically hybridize with a portion of the gene locus that flanks a target region of the locus. In one embodiment, amplification comprises using forward and reverse PCR primers directed to SEQ ID NOS: 80-82, 84, 94-145, 515, 517, 519-527, or 530-538. Nucleic acid primers useful for amplifying sequences from a FGFR fusion molecule (e.g., a FGFR1-TACC1, FGFR2-TACC2, FGFR3-TACC3 or other FGFR-TACC nucleic acid); the primers specifically hybridize with a portion of an FGFR fusion molecule. In certain subjects, the presence of an FGFR fusion molecule corresponds to a subject with a gene fusion-associated cancer. In one embodiment, amplification can comprise using forward and reverse PCR primers comprising nucleotide sequences of SEQ ID NOS: 80-82, 84, 94-145, 515, 517, 519-527, or 530-538. In one embodiment, amplification can comprise using forward and reverse PCR primers comprising nucleotide sequences of SEQ ID NOS: 162-169, or 495-514.

Non-limiting amplification methods include, e.g., polymerase chain reaction, PCR (PCR Protocols, A Guide To Methods And Applications, ed. Innis, Academic Press, N.Y., 1990 and PCR Strategies, 1995, ed. Innis, Academic Press, Inc., N.Y.); ligase chain reaction (LCR) (Wu (1989) Genomics 4:560; Landegren (1988) Science 241:1077; Barringer (1990) Gene 89:117); transcription amplification (Kwoh (1989) PNAS 86:1173); and, self-sustained sequence replication (Guatelli (1990) PNAS 87:1874); Q Beta replicase amplification (Smith (1997) J. Clin. Microbiol. 35:1477-1491), automated Q-beta replicase amplification assay (Burg (1996) Mol. Cell. Probes 10:257-271) and other RNA polymerase mediated techniques (e.g., NASBA, Cangene, Mississauga, Ontario; see also Berger (1987) Methods Enzymol. 152:307-316; U.S. Pat. Nos. 4,683,195 and 4,683,202; and Sooknanan (1995) Biotechnology 13:563-564). All the references stated above are incorporated by reference in their entireties.

The invention provides for a nucleic acid primer, wherein the primer can be complementary to and hybridize specifically to a portion of a FGFR fusion molecule, such as a FGFR1-TACC1, FGFR2-TACC2, FGFR3-TACC3 or other FGFR-TACC nucleic acid (e.g., DNA or RNA) in certain subjects having a gene fusion-associated cancer. In one embodiment, the gene-fusion associated cancer comprises glioblastoma multiforme, breast cancer, lung cancer, prostate cancer, or colorectal carcinoma. Primers of the invention can be specific for fusion sequences in a FGFR1-TACC1, FGFR2-TACC2, FGFR3-TACC3 or other FGFR-TACC nucleic acid (DNA or RNA). By using such primers, the detection of an amplification product indicates the presence of a fusion of a FGFR1 and TACC1, FGFR2 and TACC2, FGFR3 and TACC3 or other FGFR and TACC nucleic acid. Examples of primers of this invention can be single-stranded nucleic acid molecules of about 5 to 60 nucleotides in length, or about 8 to about 25 nucleotides in length. The sequence can be derived directly from the sequence of a FGFR fusion molecule, e.g. FGFR1-TACC1, FGFR2-TACC2, FGFR3-TACC3 or other FGFR-TACC nucleic acid. Perfect complementarity is useful to ensure high specificity; however, certain mismatch can be tolerated. For example, a nucleic acid primer or a pair of nucleic acid primers as described above can be used in a method for detecting the presence of a gene fusion-associated cancer in a subject. In one embodiment, primers can be used to detect an FGFR fusion molecule, such as a primer comprising SEQ ID NOS: 80-82, 84, 94-145, 515, 517, 519-527, or 530-538; or a combination thereof. In another embodiment, the PCR primers comprise SEQ ID NOS: 162, 163, 164, 165, 166, 167, 168, 169, 495, 496, 497, 498, 507, 508, 509, 510, 511, 512, 513, or 514. In a further embodiment, primers used for the screening of FGFR fusion molecules, such as FGFR-TACC fusions, comprise SEQ ID NOS: 166, 167, 168, 169, 495, 496, 497, 498, 507, 508, 509, or 510. In some embodiments, primers used for genomic detection of an FGFR3-TACC3 fusion comprise SEQ ID NOS: 170, 171, 499, 500, 501, 502, 503, 504, 505, or 506. In one embodiment, the method can comprise contacting a sample from the subject with primers specific for a FGFR fusion molecule, and determining the presence of an PCR product. In another embodiment, the method can comprise contacting a sample from the subject with primer specific for a FGFR molecule, or a TACC molecule, and determining the presence of a PCR product. In another embodiment, the primers can recognize the nucleic acids encoding a FGFR3 C-terminal region, or nucleic acids encoding a TACC3 N-terminal region, or a combination thereof. In another embodiment, the method can comprise contacting a sample from the subject with primers specific for a FGFR molecule, or a TACC molecule, or a FGFR fusion molecule, and determining the amount of PCR product formed compared to the amount of PCR product formed in non-tumor cells or tissue, wherein an increased amount of PCR product indicates the presence of an FGFR fusion. In one embodiment, primers and/or the PCR product are labeled to enable detection of the PCR product. For example, nucleic acid primers useful for amplifying sequences corresponding to a FGFR fusion molecules can be labeled with fluorescent molecules, radioactive molecules, chemiluminescent molecules, or affinity molecules (e.g. biotin) which can then be detected by methods known in the art (e.g. fluorescently labeled streptavidin). PCR products can also be detected by using dyes that can be incorporated into newly formed PCR products, such as, but not limited to, SYBR Green.

Specific Ligand Binding. As discussed herein, a nucleic acid encoding a FGFR fusion molecule or expression of a FGFR fusion molecule, can also be detected by screening for alteration(s) in a sequence or expression level of a polypeptide encoded by the same. Different types of ligands can be used, such as specific antibodies. In one embodiment, the sample is contacted with an antibody specific for a polypeptide encoded by a FGFR fusion molecule and the formation of an immune complex is subsequently determined. Various methods for detecting an immune complex can be used, such as ELISA, immunostaining, radioimmunoassays (RIA) and immuno-enzymatic assays (IEMA).

For example, an antibody can be a polyclonal antibody, a monoclonal antibody, as well as fragments or derivatives thereof having substantially the same antigen specificity. Fragments include Fab, Fab′2, or CDR regions. Derivatives include single-chain antibodies, humanized antibodies, or poly-functional antibodies. An antibody specific for a polypeptide encoded by a FGFR fusion molecule can be an antibody that selectively binds such a polypeptide. In one embodiment, the antibody is raised against a polypeptide encoded by a FGFR fusion molecule (such as FGFR1-TACC1, FGFR2-TACC2, FGFR3-TACC3 or other FGFR-TACC fusion) or an epitope-containing fragment thereof. Although non-specific binding towards other antigens can occur, binding to the target polypeptide occurs with a higher affinity and can be reliably discriminated from non-specific binding. In one embodiment, the method can comprise contacting a sample from the subject with an antibody specific for a FGFR fusion molecule, and determining the presence of an immune complex. Optionally, the sample can be contacted to a support coated with antibody specific for a FGFR fusion molecule. In one embodiment, the sample can be contacted simultaneously, or in parallel, or sequentially, with various antibodies specific for different forms of a FGFR fusion molecule, e.g., FGFR1-TACC1, FGFR2-TACC2, FGFR3-TACC3 or other FGFR-TACC fusion.

In one embodiment, the method can comprise contacting a sample from the subject with an antibody specific for a FGFR fusion molecule, and determining the presence of an immune complex. In another embodiment, the method can comprise contacting a sample from the subject with an antibody specific for a FGFR molecule, or a TACC molecule, and determining the presence of an immune complex. In another embodiment, the antibody can recognize the FGFR3 C-terminal region, or the TACC3 N-terminal region, or a combination thereof. In another embodiment, the antibody can recognize the FGFR3 C-terminal region, or the TACC3 N-terminal region, or a combination thereof. In another embodiment, the method can comprise contacting a sample from the subject with an antibody specific for a FGFR molecule, or a TACC molecule, or a FGFR fusion molecule, and determining the amount of an immune complex formed compared to the amount of immune complex formed in non-tumor cells or tissue, wherein an increased amount of an immune complex indicates the presence of an FGFR fusion.

Detection the formation of a complex between an antibody and a protein can be performed by a variety of method known in the art. For example, an antibody-protein complex can be detected by using antibodies or secondary antibodies labeled with fluorescent molecules, chromogenic molecules, chemiluminescent molecules, radioactive isotopes, or affinity molecules (e.g. biotin) which can then be detected by methods known in the art (e.g. fluorescently labeled streptavidin).

The invention also provides for a diagnostic kit comprising products and reagents for detecting in a sample from a subject the presence of a FGFR fusion molecule. The kit can be useful for determining whether a sample from a subject exhibits increased or reduced expression of a FGFR fusion molecule. For example, the diagnostic kit according to the present invention comprises any primer, any pair of primers, any nucleic acid probe and/or any ligand, or any antibody directed specifically to a FGFR fusion molecule. The diagnostic kit according to the present invention can further comprise reagents and/or protocols for performing a hybridization, amplification, or antigen-antibody immune reaction. In one embodiment, the kit can comprise nucleic acid primers that specifically hybridize to and can prime a polymerase reaction from a FGFR fusion molecule comprising SEQ ID NOS: 80-82, 84, 94-145, 515, 517, 519-527, or 530-538, or a combination thereof. In one embodiment, primers can be used to detect a FGFR fusion molecule, such as a primer directed to SEQ ID NOS: 80-82, 84, 94-145, 515, 517, 519-527, or 530-538; or a combination thereof. In another embodiment, the PCR primer comprises SEQ ID NOS: 162, 163, 164, 165, 166, 167, 168, 169, 495, 496, 497, 498, 507, 508, 509, 510, 511, 512, 513, or 514. In a further embodiment, primers used for the screening of FGFR fusion molecules, such as FGFR-TACC fusions, comprise SEQ ID NOS: 166, 167, 168, 169, 495, 496, 497, 498, 507, 508, 509, or 510. In some embodiments, primers used for genomic detection of an FGFR3-TACC3 fusion comprise SEQ ID NOS: 170, 171, 499, 500, 501, 502, 503, 504, 505, or 506. In some embodiments, the kit comprises an antibody that specifically binds to a FGFR fusion molecule comprising SEQ ID NOS: 79, 85-89, 150, 158-161, or 539-547, wherein the antibody will recognize the protein only when a FGFR fusion molecule is present. The diagnosis methods can be performed in vitro, ex vivo, or in vivo. These methods utilize a sample from the subject in order to assess the status of a FGFR fusion molecule. The sample can be any biological sample derived from a subject, which contains nucleic acids or polypeptides. Examples of such samples include, but are not limited to, fluids, tissues, cell samples, organs, and tissue biopsies. Non-limiting examples of samples include blood, liver, plasma, serum, saliva, urine, or seminal fluid. In some embodiments the sample is a tissue sample. In some embodiments, the sample is a paraffin embedded tissue section. In some embodiments, the tissue sample is a tumor sample. The sample can be collected according to conventional techniques and used directly for diagnosis or stored. The sample can be treated prior to performing the method, in order to render or improve availability of nucleic acids or polypeptides for testing. Treatments include, for instance, lysis (e.g., mechanical, physical, or chemical), centrifugation. The nucleic acids and/or polypeptides can be pre-purified or enriched by conventional techniques, and/or reduced in complexity. Nucleic acids and polypeptides can also be treated with enzymes or other chemical or physical treatments to produce fragments thereof. In one embodiment, the sample is contacted with reagents, such as probes, primers, or ligands, in order to assess the presence of a FGFR fusion molecule. Contacting can be performed in any suitable device, such as a plate, tube, well, or glass. In some embodiments, the contacting is performed on a substrate coated with the reagent, such as a nucleic acid array or a specific ligand array. The substrate can be a solid or semi-solid substrate such as any support comprising glass, plastic, nylon, paper, metal, or polymers. The substrate can be of various forms and sizes, such as a slide, a membrane, a bead, a column, or a gel. The contacting can be made under any condition suitable for a complex to be formed between the reagent and the nucleic acids or polypeptides of the sample.

### Nucleic Acid Delivery Methods

Delivery of nucleic acids into viable cells can be effected ex vivo, in situ, or in vivo by use of vectors, such as viral vectors (e.g., lentivirus, adenovirus, adeno-associated virus, or a retrovirus), or ex vivo by use of physical DNA transfer methods (e.g., liposomes or chemical treatments). Non-limiting techniques suitable for the transfer of nucleic acid into mammalian cells in vitro include the use of liposomes, electroporation, microinjection, cell fusion, DEAE-dextran, and the calcium phosphate precipitation method (See, for example, Anderson, Nature, 1998) supplement to 392(6679):25( ). Introduction of a nucleic acid or a gene encoding a polypeptide of the invention can also be accomplished with extrachromosomal substrates (transient expression) or artificial chromosomes (stable expression). Cells can also be cultured ex vivo in the presence of therapeutic compositions of the present invention in order to proliferate or to produce a desired effect on or activity in such cells. Treated cells can then be introduced in vivo for therapeutic purposes.

Nucleic acids can be inserted into vectors and used as gene therapy vectors. A number of viruses have been used as gene transfer vectors, including papovaviruses, e.g., SV40 (Madzak et al., (1992) J Gen Virol. 73(Pt 6):1533-6), adenovirus (Berkner (1992) Curr Top Microbiol Immunol. 158:39-66; Berkner (1988) Biotechniques, 6(7):616-29; Gorziglia and Kapikian (1992) J Virol. 66(7):4407-12; Quantin et al., (1992) Proc Natl Acad Sci USA. 89(7):2581-4; Rosenfeld et al., (1992) Cell. 68(1):143-55; Wilkinson et al., (1992) Nucleic Acids Res. 20(9):2233-9; Stratford-Perricaudet et al., (1990) Hum Gene Ther. 1(3):241-56), vaccinia virus (Moss (1992) Curr Opin Biotechnol. 3(5):518-22), adeno-associated virus (Muzyczka, (1992) Curr Top Microbiol Immunol. 158:97-129; Ohi et al., (1990) Gene. 89(2):279-82), herpesviruses including HSV and EBV (Margolskee (1992) Curr Top Microbiol Immunol. 158:67-95; Johnson et al., (1992) Brain Res Mol Brain Res. 12(1-3):95-102; Fink et al., (1992) Hum Gene Ther. 3(1):11-9; Breakefield and Geller (1987) Mol Neurobiol. 1(4):339-71; Freese et al., (1990) Biochem Pharmacol. 40(10):2189-99), and retroviruses of avian (Bandyopadhyay and Temin (1984) Mol Cell Biol. 4(4):749-54; Petropoulos et al., (1992) J Virol. 66(6):3391-7), murine (Miller et al. (1992) Mol Cell Biol. 12(7):3262-72; Miller et al., (1985) J Virol. 55(3):521-6; Sorge et al., (1984) Mol Cell Biol. 4(9):1730-7; Mann and Baltimore (1985) J Virol. 54(2):401-7; Miller et al., (1988) J Virol. 62(11):4337-45), and human origin (Shimada et al., (1991) J Clin Invest. 88(3):1043-7; Helseth et al., (1990) J Virol. 64(12):6314-8; Page et al., (1990) J Virol. 64(11):5270-6; Buchschacher and Panganiban (1992) J Virol. 66(5):2731-9).

Non-limiting examples of in vivo gene transfer techniques include transfection with viral (e.g., retroviral) vectors (see U.S. Pat. No. 5,252,479, which is incorporated by reference in its entirety) and viral coat protein-liposome mediated transfection (Dzau et al., (1993) Trends in Biotechnology 11:205-210), incorporated entirely by reference). For example, naked DNA vaccines are generally known in the art; see Brower, (1998) Nature Biotechnology, 16:1304-1305, which is incorporated by reference in its entirety. Gene therapy vectors can be delivered to a subject by, for example, intravenous injection, local administration (see, e.g., U.S. Pat. No. 5,328,470) or by stereotactic injection (see, e.g., Chen, et al., (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

For reviews of nucleic acid delivery protocols and methods see Anderson et al. (1992) Science 256:808-813; U.S. Pat. Nos. 5,252,479, 5,747,469, 6,017,524, 6,143,290, 6,410,010 6,511,847; and U.S. Application Publication No. 2002/0077313, which are all hereby incorporated by reference in their entireties. For additional reviews, see Friedmann (1989) Science, 244:1275-1281; Verma, Scientific American: 68-84 (1990); Miller (1992) Nature, 357: 455-460; Kikuchi et al. (2008) J Dermatol Sci. 50(2):87-98; Isaka et al. (2007) Expert Opin Drug Deliv. 4(5):561-71; Jager et al. (2007) Curr Gene Ther. 7(4):272-83; Waehler et al. (2007) Nat Rev Genet. 8(8):573-87; Jensen et al. (2007) Ann Med. 39(2):108-15; Herweijer et al. (2007) Gene Ther. 14(2):99-107; Eliyahu et al. (2005) Molecules 10(1):34-64; and Altaras et al. (2005) Adv Biochem Eng Biotechnol. 99:193-260, all of which are hereby incorporated by reference in their entireties.

A FGFR fusion nucleic acid can also be delivered in a controlled release system. For example, the FGFR fusion molecule can be administered using intravenous infusion, an implantable osmotic pump, a transdermal patch, liposomes, or other modes of administration. In one embodiment, a pump can be used (see Sefton (1987) Biomed. Eng. 14:201; Buchwald et al. (1980) Surgery 88:507; Saudek et al. (1989) N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, (1983) J. Macromol. Sci. Rev. Macromol. Chem. 23:61; see also Levy et al. (1985) Science 228:190; During et al. (1989) Ann. Neurol. 25:351; Howard et al. (1989) J. Neurosurg. 71:105). In yet another embodiment, a controlled release system can be placed in proximity of the therapeutic target thus requiring only a fraction of the systemic dose (see, e.g., Goodson, in Medical Applications of Controlled Release, supra, vol. 2, pp. 115-138 (1984)). Other controlled release systems are discussed in the review by Langer (Science (1990) 249:1527-1533).

### Pharmaceutical Compositions and Administration for Therapy

An inhibitor of the invention can be incorporated into pharmaceutical compositions suitable for administration, for example the inhibitor and a pharmaceutically acceptable carrier

A FGFR fusion molecule or inhibitor of the invention (e.g. JNJ-42756493) can be administered to the subject once (e.g., as a single injection or deposition). Alternatively, a FGFR fusion molecule or inhibitor can be administered once or twice daily to a subject in need thereof for a period of from about two to about twenty-eight days, or from about seven to about ten days. A FGFR fusion molecule or inhibitor can also be administered once or twice daily to a subject for a period of 1, 2, 3, 4, 5, 6, 7, 8, 9, 10, 11, 12 times per year, or a combination thereof. A FGFR fusion molecule or inhibitor can also be administered in seven to ten day repeating cycles (i.e. administration of a FGFR fusion molecule or inhibitor for seven to ten days, followed by no administration of a FGFR fusion molecule or inhibitor for seven to ten days). Furthermore, a FGFR fusion molecule or inhibitor of the invention can be co-administrated with another therapeutic. Where a dosage regimen comprises multiple administrations, the effective amount of the FGFR fusion molecule or inhibitor administered to the subject can comprise the total amount of gene product administered over the entire dosage regimen.

A FGFR fusion molecule or inhibitor can be administered to a subject by any means suitable for delivering the FGFR fusion molecule or inhibitor to cells of the subject, such as cancer cells, e.g., glioblastoma multiforme, breast cancer, lung cancer, prostate cancer, colorectal carcinoma, bladder carcinoma, squamous lung carcinoma, head and neck carcinoma, glioma, grade II or III glioma, or IDH wild-type grade II or III glioma. For example, a FGFR fusion molecule or inhibitor can be administered by methods suitable to transfect cells. Transfection methods for eukaryotic cells are well known in the art, and include direct injection of the nucleic acid into the nucleus or pronucleus of a cell; electroporation; liposome transfer or transfer mediated by lipophilic materials; receptor mediated nucleic acid delivery, bioballistic or particle acceleration; calcium phosphate precipitation, and transfection mediated by viral vectors.

The compositions of this invention can be formulated and administered to reduce the symptoms associated with a gene fusion-associated cancer, e.g., glioblastoma multiforme, breast cancer, lung cancer, prostate cancer, colorectal carcinoma, bladder carcinoma, squamous lung carcinoma, head and neck carcinoma, glioma, grade II or III glioma, or IDH wild-type grade II or III glioma, by any means that produces contact of the active ingredient with the agent's site of action in the body of a subject, such as a human or animal (e.g., a dog, cat, or horse). They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

A therapeutically effective dose of FGFR fusion molecule or inhibitor (e.g. JNJ-42756493) can depend upon a number of factors known to those or ordinary skill in the art. The dose(s) of the FGFR fusion molecule inhibitor can vary, for example, depending upon the identity, size, and condition of the subject or sample being treated, further depending upon the route by which the composition is to be administered, if applicable, and the effect which the practitioner desires the a FGFR fusion molecule inhibitor to have upon the nucleic acid or polypeptide of the invention. For example, 12 mg of JNJ-42756493 can be orally administered daily. JNJ-42756493 can be administered in seven to ten day repeating cycles (i.e. administration of JNJ-42756493 for seven to ten days, followed by no administration of JNJ-42756493 for seven to ten days). These amounts can be readily determined by a skilled artisan. Any of the therapeutic applications described herein can be applied to any subject in need of such therapy, including, for example, a mammal such as a dog, a cat, a cow, a horse, a rabbit, a monkey, a pig, a sheep, a goat, or a human.

Pharmaceutical compositions for use in accordance with the invention can be formulated in conventional manner using one or more physiologically acceptable carriers or excipients. The therapeutic compositions of the invention can be formulated for a variety of routes of administration, including systemic and topical or localized administration. Techniques and formulations generally can be found in Remington's Pharmaceutical Sciences, Meade Publishing Co., Easton, Pa (20^{th} Ed., 2000), the entire disclosure of which is herein incorporated by reference. For systemic administration, an injection is useful, including intramuscular, intravenous, intraperitoneal, and subcutaneous. For injection, the therapeutic compositions of the invention can be formulated in liquid solutions, for example in physiologically compatible buffers such as Hank's solution or Ringer's solution. In addition, the therapeutic compositions can be formulated in solid form and redissolved or suspended immediately prior to use. Lyophilized forms are also included. Pharmaceutical compositions of the present invention are characterized as being at least sterile and pyrogen-free. These pharmaceutical formulations include formulations for human and veterinary use.

According to the invention, a pharmaceutically acceptable carrier can comprise any and all solvents, dispersion media, coatings, antibacterial and antifungal agents, isotonic and absorption delaying agents, and the like, compatible with pharmaceutical administration. The use of such media and agents for pharmaceutically active substances is well known in the art. Any conventional media or agent that is compatible with the active compound can be used. Supplementary active compounds can also be incorporated into the compositions.

A pharmaceutical composition containing FGFR fusion molecule inhibitor can be administered in conjunction with a pharmaceutically acceptable carrier, for any of the therapeutic effects discussed herein. Such pharmaceutical compositions can comprise, for example antibodies directed to a FGFR fusion molecule, or a variant thereof, or antagonists of a FGFR fusion molecule, or JNJ-42756493. The compositions can be administered alone or in combination with at least one other agent, such as a stabilizing compound, which can be administered in any sterile, biocompatible pharmaceutical carrier including, but not limited to, saline, buffered saline, dextrose, and water. The compositions can be administered to a patient alone, or in combination with other agents, drugs or hormones.

Sterile injectable solutions can be prepared by incorporating the FGFR fusion molecule inhibitor (e.g., a polypeptide or antibody) in the required amount in an appropriate solvent with one or a combination of ingredients enumerated herein, as required, followed by filtered sterilization. Generally, dispersions are prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional desired ingredient from a previously sterile-filtered solution thereof.

In some embodiments, the FGFR fusion molecule inhibitor can be applied via transdermal delivery systems, which slowly releases the active compound for percutaneous absorption. Permeation enhancers can be used to facilitate transdermal penetration of the active factors in the conditioned media. Transdermal patches are described in for example, U.S. Pat. Nos. 5,407,713; 5,352,456; 5,332,213; 5,336,168; 5,290,561; 5,254,346; 5,164,189; 5,163,899; 5,088,977; 5,087,240; 5,008,110; and 4,921,475.

“Subcutaneous” administration can refer to administration just beneath the skin (i.e., beneath the dermis). Generally, the subcutaneous tissue is a layer of fat and connective tissue that houses larger blood vessels and nerves. The size of this layer varies throughout the body and from person to person. The interface between the subcutaneous and muscle layers can be encompassed by subcutaneous administration. This mode of administration can be feasible where the subcutaneous layer is sufficiently thin so that the factors present in the compositions can migrate or diffuse from the locus of administration. Thus, where intradermal administration is utilized, the bolus of composition administered is localized proximate to the subcutaneous layer.

Administration of the cell aggregates (such as DP or DS aggregates) is not restricted to a single route, but can encompass administration by multiple routes. For instance, exemplary administrations by multiple routes include, among others, a combination of intradermal and intramuscular administration, or intradermal and subcutaneous administration. Multiple administrations can be sequential or concurrent. Other modes of application by multiple routes will be apparent to the skilled artisan.

In other embodiments, this implantation method will be a one-time treatment for some subjects. In further embodiments of the invention, multiple cell therapy implantations will be required. In some embodiments, the cells used for implantation will generally be subject-specific genetically engineered cells. In another embodiment, cells obtained from a different species or another individual of the same species can be used. Thus, using such cells can require administering an immunosuppressant to prevent rejection of the implanted cells. Such methods have also been described in U.S. Pat. No. 7,419,661 and PCT application publication WO 2001/32840, and are hereby incorporated by reference.

A pharmaceutical composition of the invention is formulated to be compatible with its intended route of administration. Examples of routes of administration include parenteral, e.g., intravenous, intradermal, subcutaneous, oral (e.g., inhalation or ingestion), transdermal (topical), transmucosal, and rectal administration. For example, JNJ-42756493 can be orally administered. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it can be useful to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the inhibitor (e.g., a polypeptide or antibody or small molecule) of the invention in the required amount in an appropriate solvent with one or a combination of ingredients enumerated herein, as required, followed by filtered sterilization. Generally, dispersions are prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional desired ingredient from a previously sterile-filtered solution thereof.

Oral compositions (e.g. of JNJ-42756493) generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier and subsequently swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In some embodiments, the effective amount of the administered FGFR fusion molecule inhibitor (e.g. JNJ-42756493) is at least about 0.0001 μg/kg body weight, at least about 0.00025 μg/kg body weight, at least about 0.0005 μg/kg body weight, at least about 0.00075 g/kg body weight, at least about 0.001 μg/kg body weight, at least about 0.0025 μg/kg body weight, at least about 0.005 μg/kg body weight, at least about 0.0075 μg/kg body weight, at least about 0.01 μg/kg body weight, at least about 0.025 μg/kg body weight, at least about 0.05 μg/kg body weight, at least about 0.075 μg/kg body weight, at least about 0.1 μg/kg body weight, at least about 0.25 μg/kg body weight, at least about 0.5 μg/kg body weight, at least about 0.75 g/kg body weight, at least about 1 μg/kg body weight, at least about 5 μg/kg body weight, at least about 10 μg/kg body weight, at least about 25 μg/kg body weight, at least about 50 μg/kg body weight, at least about 75 μg/kg body weight, at least about 100 μg/kg body weight, at least about 150 μg/kg body weight, at least about 200 μg/kg body weight, at least about 250 μg/kg body weight, at least about 300 μg/kg body weight, at least about 350 μg/kg body weight, at least about 400 μg/kg body weight, at least about 450 μg/kg body weight, at least about 500 μg/kg body weight, at least about 550 μg/kg body weight, at least about 600 μg/kg body weight, at least about 650 μg/kg body weight, at least about 700 μg/kg body weight, at least about 750 μg/kg body weight, at least about 800 μg/kg body weight, at least about 850 μg/kg body weight, at least about 900 μg/kg body weight, at least about 950 μg/kg body weight, at least about 1,000 μg/kg body weight, at least about 2,000 μg/kg body weight, at least about 3,000 μg/kg body weight, at least about 4,000 μg/kg body weight, at least about 5,000 μg/kg body weight, at least about 6,000 g/kg body weight, at least about 7,000 μg/kg body weight, at least about 8,000 μg/kg body weight, at least about 9,500 μg/kg body weight, or at least about 10,000 μg/kg body weight.

In some embodiments, the effective amount of the administered FGFR fusion molecule inhibitor (e.g. JNJ-42756493) is at least about 1 mg, 2 mg, 3 mg, 4 mg, 5 mg, 6 mg, 7 mg, 8 mg, 9 mg, 10 mg, 11 mg, 12 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg.

Unless otherwise defined, all technical and scientific terms used herein have the same meaning as commonly understood by one of ordinary skill in the art to which this invention belongs. Exemplary methods and materials are described below, although methods and materials similar or equivalent to those described herein can also be used in the practice or testing of the present invention.

All publications and other references mentioned herein are incorporated by reference in their entirety, as if each individual publication or reference were specifically and individually indicated to be incorporated by reference. Publications and references cited herein are not admitted to be prior art.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

The invention is further illustrated in Singh et al., Science (2012), 337(6099):1231-5 (including the accompanying Supplementary Information). The entire contents of Singh et al., Science (2012), 337(6099):1231-5, including the accompanying “Supplementary Information,” is expressly incorporated by reference. The invention is also further illustrated in Di Stefano et al., “Detection, characterization and inhibition of FGFR-TACC fusions in IDH wild type glioma” Clin. Cancer Res. (2015), the entire contents of which are expressly incorporated by reference.

### Example 1: Transforming and Recurrent Fusions of FGFR and TACC Gene in Glioblastoma

The history of successful targeted therapy of cancer largely coincides with the inactivation of recurrent, oncogenic and addicting gene fusions in hematological malignancies and recently in some types of epithelial cancer. Glioblastoma multiforme (GBM) is among the most lethal forms of human cancer. Here, an integrated gene fusion discovery pipeline was developed for the detection of in-frame fused transcripts from RNA-seq and genomic fusions from whole exome sequences. The application of the pipeline to human GBM unraveled recurrent chromosomal translocations, which fuse in-frame the tyrosine kinase domain of FGFR genes (FGFR1 or FGFR3) to the TACC domain of TACC1 or TACC3, respectively. The frequency of FGFR-TACC fusions is 3 of 97 GBM (3.1%). The FGFR-TACC fusion protein displays strong oncogenic activity when introduced into astrocytes or transduced by lentivirus-mediated stereotactic delivery to the adult mouse brain. The FGFR-TACC fusion protein mis-localizes over the mitotic spindle pole, has constitutive tyrosine kinase activity and dysregulates the mitotic cycle with delayed mitotic progression. The impaired mitotic fidelity triggers chromatid cohesion defects, defective spindle checkpoint activation, chromosomal mis-segregation, and rampant aneuploidy. Inhibition of FGFR kinase corrects the aneuploidy and oral administration of a specific FGFR tyrosine kinase inhibitor under clinical investigation arrests tumor growth and prolongs survival of mice harboring intracranial FGFR3-TACC3-initiated glioma. FGFR-TACC fusions identify a subset of GBM patients who may benefit from targeted inhibition of the tyrosine kinase activity of FGFR.

Glioblastoma multiforme (GBM) is among the most difficult forms of cancer to treat in humans (Ohgaki and Kleihues, 2005). So far, the targeted therapeutic approaches that have been tested against potentially important oncogenic drivers in GBM have met limited success (Lo, 2010; Reardon et al., 2010; van den Bent et al., 2009). Recurrent chromosomal translocations leading to production of oncogenic fusion proteins are viewed as initiating and addicting events in the pathogenesis of human cancer, thus providing the most desirable molecular targets for cancer therapy (Ablain et al., 2011; Mitelman et al., 2007). Chromosomal rearrangements resulting in recurrent and oncogenic gene fusions are hallmarks of hematological malignancies and recently they have also been uncovered in subsets of solid tumors (breast, prostate, lung and colorectal carcinoma), but they have not been found in GBM (Bass et al., 2011; Prensner and Chinnaiyan, 2009). Important and successful targeted therapeutic interventions for patients whose tumors carry these rearrangements have stemmed from the discovery of functional gene fusions, especially when the translocations involve kinase-coding genes (BCR-ABL, EML4-ALK) (Druker, 2009; Gerber and Minna, 2010).

A hallmark of GBM is rampant chromosomal instability (CIN), which leads to aneuploidy (Furnari et al., 2007). CIN and aneuploidy are early events in the pathogenesis of cancer (Cahill et al., 1999). It has been suggested that genetic alterations targeting mitotic fidelity might be responsible for mis-segregation of chromosomes during mitosis, resulting in aneuploidy (Gordon et al., 2012; Solomon et al., 2011). Here, the first cases of recurrent and oncogenic gene fusions in human GBM are described. The resulting fusion protein localizes to mitotic cells, disrupts the normal control of chromosome segregation and induces aneuploidy. A therapeutic strategy with FGFR tyrosine kinase inhibitors is also reported for the targeted therapy of GBM patients harboring these chromosomal rearrangements.

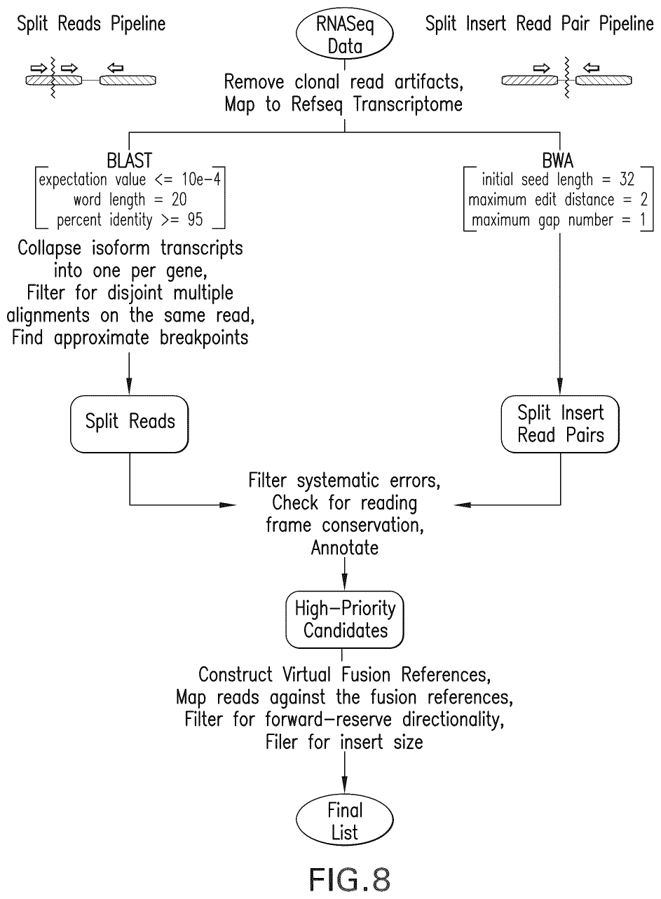

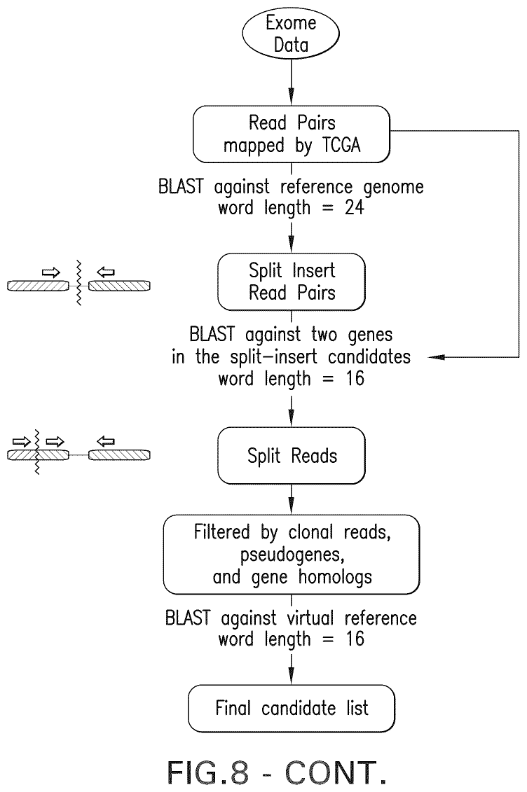

Identification of recurrent fusions of FGFR and TACC genes. To identify genomic rearrangements in GBM that generate functional fusion proteins and are recurrent, gene pairs discovered as in-frame fused transcripts from the analysis of massively parallel, paired-end sequencing of expressed transcripts (RNA-seq) would also emerge as fused gene pairs from the genomic analysis of human GBM. Towards this aim, two complementary gene fusion discovery methods were devised and were applied to two GBM cohorts. The first, TX-Fuse, is an algorithm for the discovery of candidate fusion transcripts from RNA-seq (FIG. 8). The second, Exome-Fuse, detects fusion genes from whole exome DNA sequences (FIG. 8). As first step for the detection of fused transcripts, RNA-seq data was generated from short-term cultures of glioma stem-like cells (GSCs) freshly isolated from nine patients carrying primary GBM. The culture of primary GBM tumors under serum-free conditions selects cells that retain phenotypes and genotypes closely mirroring primary tumor profiles as compared to serum-cultured glioma cell lines that have largely lost their developmental identities (Lee et al., 2006). Therefore, without being bound by theory, if glioma cells carry gene fusions causally responsible for the most aggressive hallmarks of GBM, they should be selected in GSCs. RNA-seq generated an average of 60.3 million paired reads for each GSC culture, of which over 80% were mapped to the reference transcriptome and genome. TX-Fuse detects two main sources of evidence: split reads and split inserts (see Experimental Procedures). The application of TX-Fuse to the RNA-seq dataset from nine GSCs led to the discovery of five candidate rearrangements (all of which were intrachromosomal) that give rise to in-frame fusion transcripts (Table 1B).

TABLE 1B
Predicted in-frame fusion proteins from RNA-Seq of nine GSCs

| # Split | # Split | | | | | | | |
|---|---|---|---|---|---|---|---|---|
| Inserts | Reads | Sample | Gene1 | Gene2 | Ref Seq1 | Ref Seq2 | Tx Pos1 | Tx Pos2 |
| 294 | 76 | GSC-1123 | FGFR3 | TACC3 | NM_000142 | NM_006342 | 2530 | 1751 |
| 37 | 54 | GSC-0114 | POLR2A | WRAP53 | NM_000937 | NM_001143990 | 479 | 798 |
| 7 | 48 | GSC-0114 | CAPZB | UBR4 | NM_001206540 | NM_020765 | 228 | 12111 |
| 8 | 29 | GSC-0517 | ST8SIA4 | PAM | NM_005668 | NM_000919 | 1125 | 730 |
| 6 | 17 | GSC-0308 | PIGU | NCOA6 | NM_080476 | NM_014071 | 729 | 6471 |
| 1 | 6 | GSC-0127 | IFNAR2 | IL10RB | NM_000874 | NM_000628 | 1083 | 149 |

| #Split | #Split | | | | | | | |
|---|---|---|---|---|---|---|---|---|
| Inserts | Reads | Sample | Chr 1 | Strand 1 | hg19_GenPos1 | Chr 2 | Strand 2 | hg19_GenPos2 |
| 294 | 76 | GSC-1123 | 4 | + | 1808842 | 4 | + | 1737004 |
| 37 | 54 | GSC-0114 | 17 | + | 7399259 | 17 | + | 7604059 |
| 7 | 48 | GSC-0114 | 1 | − | 19712098 | 1 | − | 19433440 |
| 8 | 29 | GSC-0517 | 5 | − | 100147809 | 5 | + | 102260661 |
| 6 | 17 | GSC-0308 | 20 | − | 33203914 | 20 | − | 33303130 |
| 1 | 6 | GSC-0127 | 21 | + | 34632901 | 21 | + | 34640699 |

Next, genomic rearrangements leading to gene fusions were identified in GBM by applying Exome-Fuse to a dataset of paired-end exome DNA sequences from 84 GBM samples from TCGA (Table 2).

This analysis detected 147 paired gene fusions, thus producing an average of 1.75 gene fusion events per tumor (Table 3).

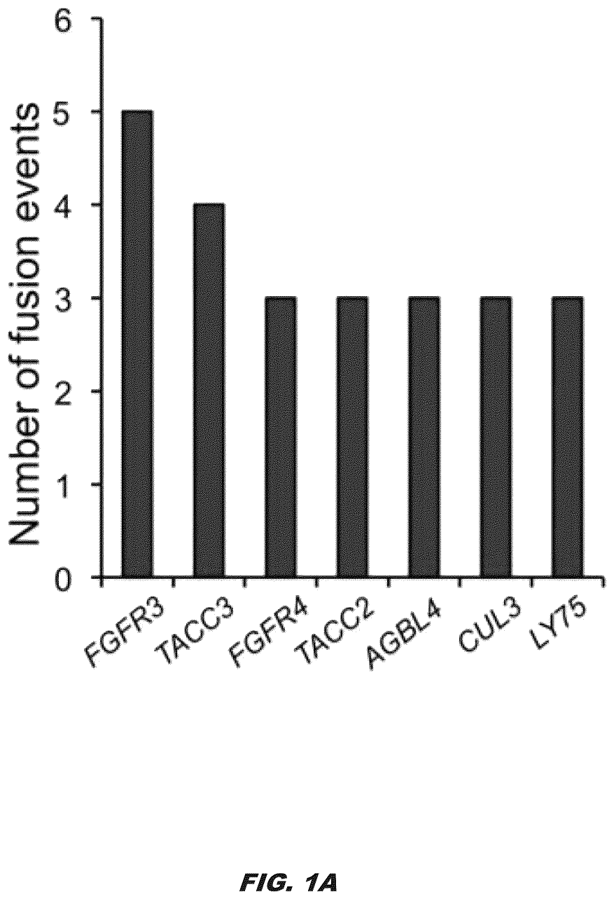

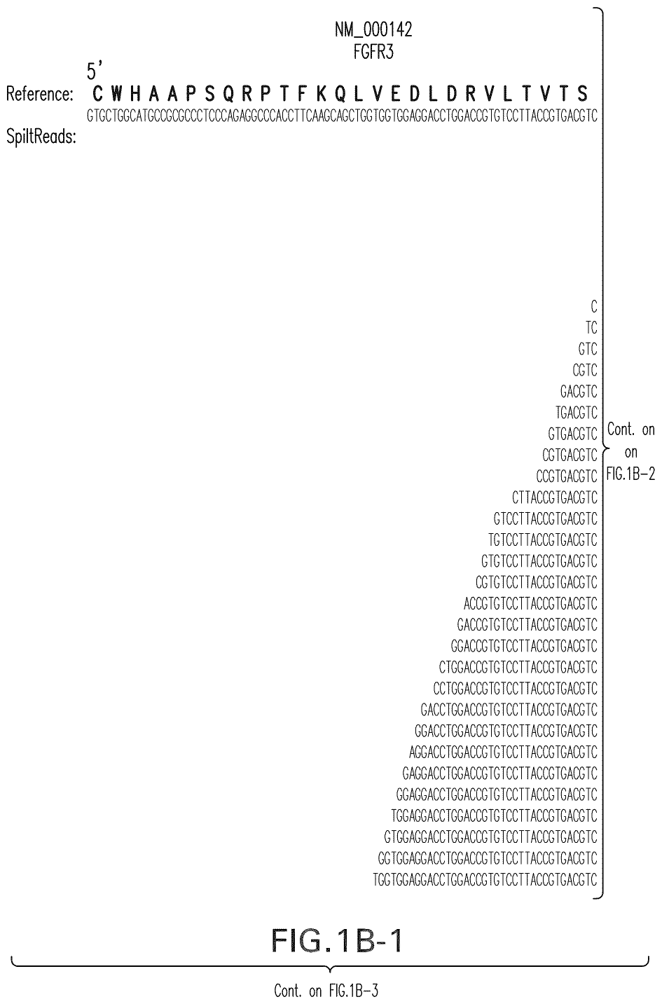

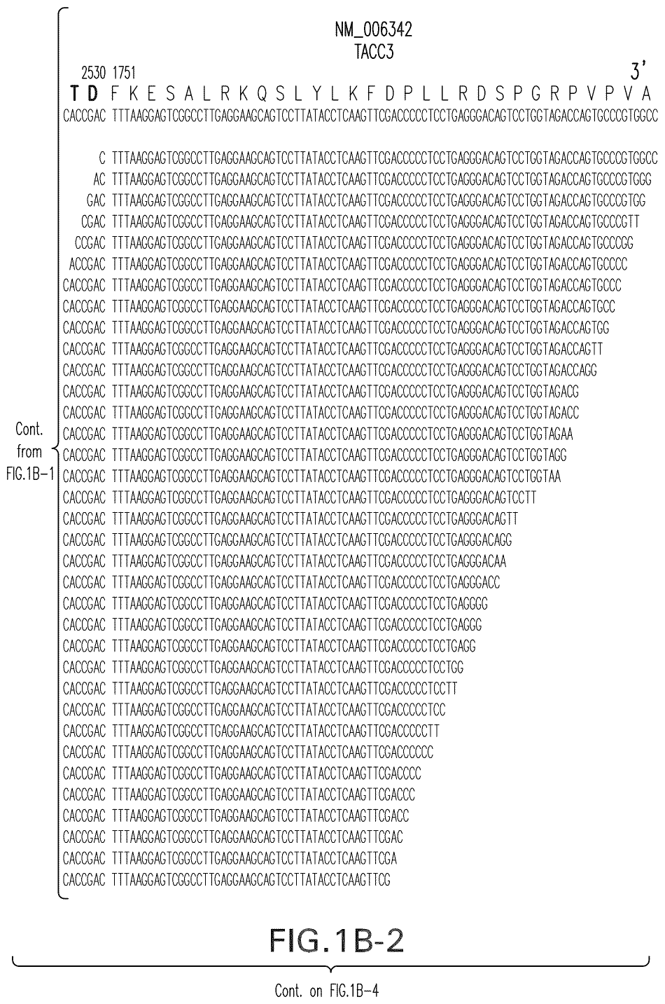

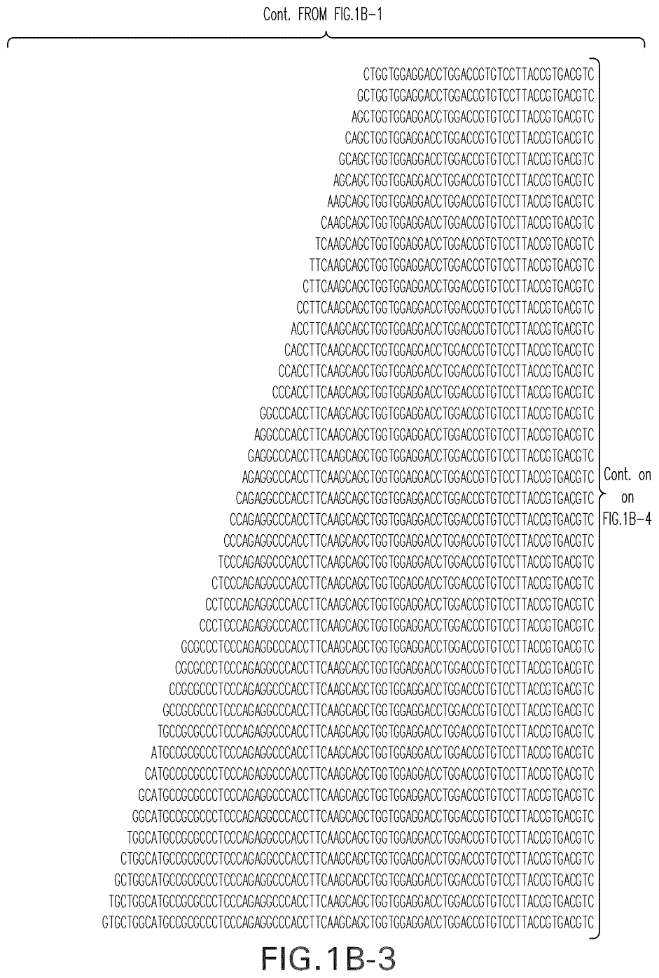

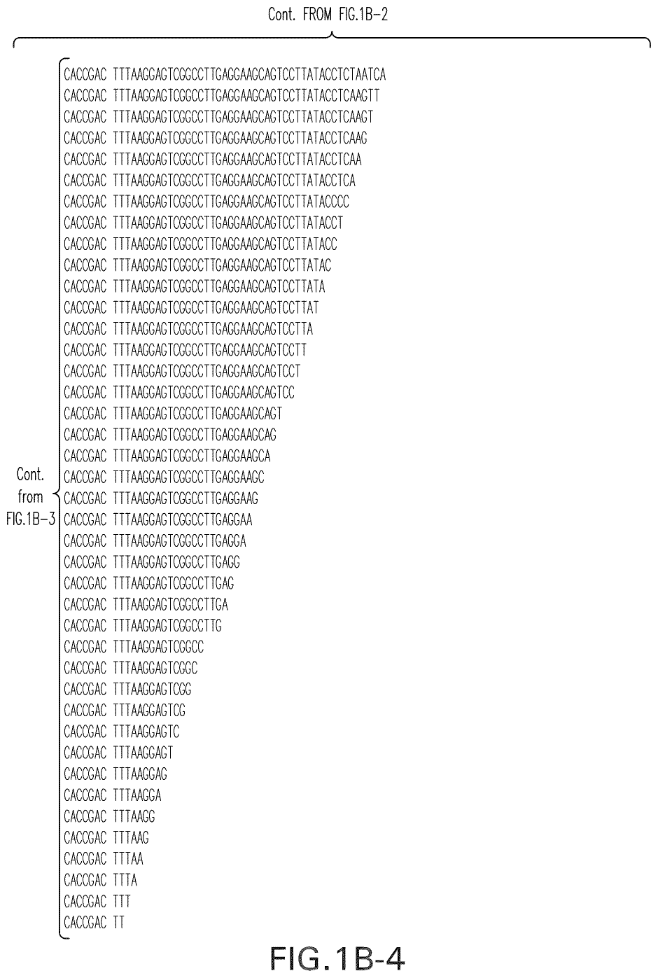

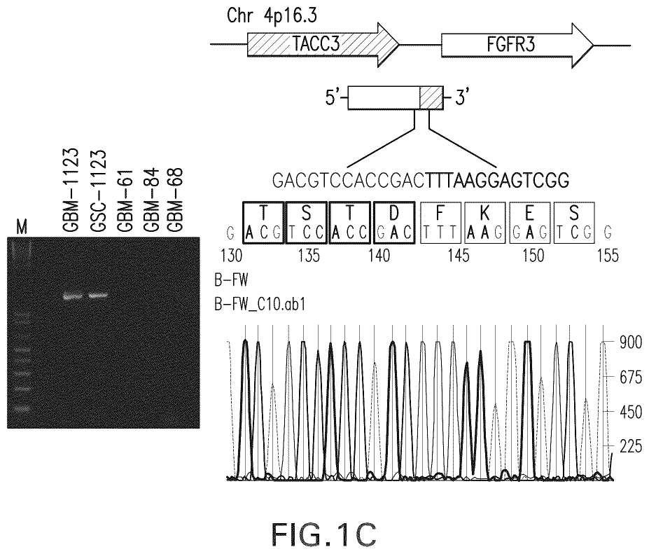

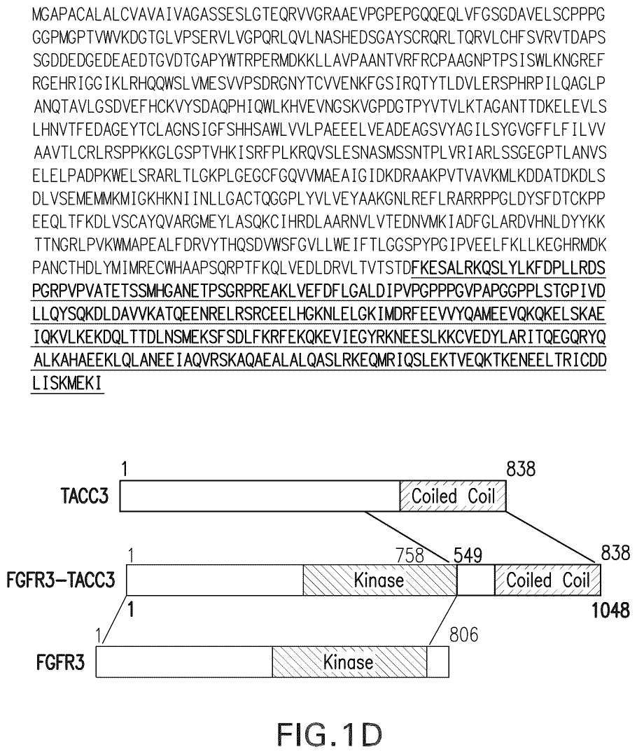

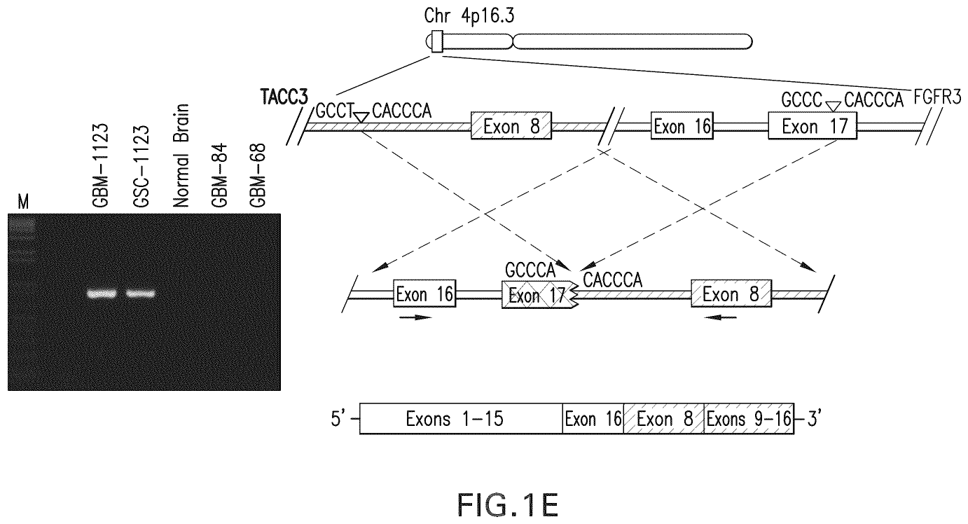

The FGFR and TACC families of genes were markedly enriched among those recurrently involved in genomic fusions, with eight tumors harboring FGFR rearrangements and seven tumors harboring fusions that implicate TACC genes (FIG. 1A). The comparative analysis of the TX-Fuse and Exon-Fuse outputs revealed that FGFR3-TACC3 was the only fusion pair identified as either an in-frame transcript by TX-Fuse and genomic fusions by Exome-Fuse (Tables 1B, 2 and 3).

Table 2 shows fusion breakpoint information of recurrent gene fusions identified by Exome-fuse analysis of 84 GBM from TCGA. As multiple junctions may exist in each fusion candidate, information for all breakpoints is displayed. Column definitions include: sample=TCGA sample ID, virtForSplitReads/virtRevSplitReads/virtTotSplitReads=#forward/reverse/total split reads, splitInserts=#split inserts, dirA/dirB=forward (1) or reverse (0) direction of split read portion mapping to gene A/B, dirAB_matepair=direction of mate pair of split read, cosmicA+B=#recorded mutations of gene A+B in COSMIC.

TABLE 2
Fusion breakpoint information of recurrent gene fusions identified by Exome-fuse analysis of 84 GBM from TCGA.

| | | | | | | | | | | | | | | | | cosmic |
|---|---|---|---|---|---|---|---|---|---|---|---|---|---|---|---|---|
| Sample | vlrtForSplitReads | vlrtRevSplitReads | vlrtForSplitReads | splitinserts | geneA | chrA | senseA | posA | geneB | chrB | senseB | posB | chrA | chrB | dirAB_matepair | A + B |
| TCGA-06-6390 | 10 | 9 | 19 | 8 | FGFR3 | chr4 | + | 1778521 | TACC3 | chr4 | + | 1708787 | 1 | 1 | 0 | 2803 |
| TCGA-12-0826 | 5 | 6 | 11 | 5 | FGFR3 | chr4 | + | 1778502 | TACC3 | chr4 | + | 1707185 | 0 | 0 | 1 | 2803 |
| TCGA-19-5958 | 3 | 0 | 3 | 2 | FGFR3 | chr4 | + | 1778539 | TACC3 | chr4 | + | 1707203 | 0 | 1 | 1 | 2803 |
| TCGA-27-1835 | 11 | 1 | 12 | 4 | FGFR3 | chr4 | + | 1778595 | TACC3 | chr4 | + | 1709397 | 0 | 0 | 1 | 2803 |
| TCGA-12-0820 | 7 | 2 | 9 | 4 | FGFR3 | chr4 | + | 1779184 | PRKG2 | chr4 | − | 82338347 | 1 | 1 | 0 | 2805 |
| TCGA-12-1088 | 3 | 1 | 4 | 4 | ABL1 | chr9 | + | 132597569 | TNFRSF10B | chr8 | − | 22936252 | 0 | 0 | 1 | 892 |
| TCGA-06-1802 | 7 | 1 | 8 | 8 | ADAM12 | chr10 | − | 127698245 | PTPRD | chr9 | − | 8596127 | 0 | 0 | 1 | 54 |
| TCGA-06-1801 | 7 | 0 | 7 | 5 | HIP1 | chr7 | − | 75010010 | PTPRD | chr9 | − | 9387093 | 1 | 0 | 0 | 52 |
| TCGA-12-1088 | 3 | 0 | 3 | 3 | KIDINS220 | chr2 | − | 8886300 | PPP1R3A | chr7 | − | 113305567 | 0 | 0 | 1 | 45 |
| TCGA-12-1088 | 37 | 1 | 38 | 10 | KIDINS220 | chr2 | − | 8887075 | PPP1R3A | chr7 | − | 113305191 | 1 | 1 | 0 | 45 |
| TCGA-32-2491 | 2 | 17 | 19 | 6 | ODZ1 | chrX | − | 123342503 | STAG2 | chrX | + | 123019118 | 0 | 1 | 0 | 36 |
| TCGA-32-2491 | 11 | 1 | 12 | 10 | ODZ1 | chrX | − | 123526882 | SASH3 | chrX | + | 128749198 | 1 | 0 | 0 | 34 |
| TCGA-12-0829 | 24 | 0 | 24 | 13 | LRRK2 | chr12 | + | 39032542 | VSNL1 | chr2 | + | 17630556 | 1 | 1 | 0 | 32 |
| TCGA-12-0829 | 25 | 1 | 26 | 13 | LRRK2 | chr12 | + | 38975444 | VSNL1 | chr2 | + | 17639377 | 1 | 0 | 0 | 32 |
| TCGA-12-0829 | 87 | 16 | 103 | 58 | LRRK2 | chr12 | + | 38975652 | VSNL1 | chr2 | + | 17639552 | 0 | 1 | 1 | 32 |
| TCGA-19-0957 | 3 | 2 | 5 | 6 | NUDT19 | chr19 | + | 37891921 | ODZ1 | chrX | − | 123925223 | 1 | 0 | 0 | 32 |
| TCGA-12-1088 | 12 | 1 | 13 | 5 | GLI3 | chr7 | − | 42031380 | RIMBP2 | chr12 | − | 129517282 | 1 | 0 | 0 | 31 |
| TCGA-12-1088 | 5 | 0 | 5 | 1 | GLI3 | chr7 | − | 42031574 | RIMBP2 | chr12 | − | 129517455 | 0 | 1 | 1 | 31 |
| TCGA-12-1089 | 10 | 0 | 10 | 5 | AHNAK | chr11 | − | 62056459 | C21orf29 | chr21 | − | 44923276 | 0 | 1 | 1 | 30 |
| TCGA-06-1801 | 27 | 1 | 28 | 12 | CROCC | chr1 | + | 17171362 | CSMD2 | chr1 | − | 34381139 | 1 | 0 | 0 | 29 |
| TCGA-12-1089 | 12 | 1 | 13 | 6 | CLK3 | chr15 | + | 72705401 | LRP1 | chr12 | + | 55880002 | 0 | 1 | 1 | 28 |
| TCGA-12-1089 | 14 | 2 | 16 | 8 | CLK3 | chr15 | + | 72705248 | LRP1 | chr12 | + | 55879646 | 1 | 0 | 0 | 28 |
| TCGA-12-1089 | 42 | 5 | 47 | 24 | LAMA2 | chr6 | + | 129836071 | PDE10A | chr6 | − | 165858426 | 1 | 0 | 0 | 28 |
| TCGA-06-1802 | 48 | 9 | 57 | 27 | LAMA2 | chr6 | + | 129483265 | SEC14L3 | chr22 | − | 29193005 | 1 | 1 | 0 | 27 |
| TCGA-19-0957 | 4 | 16 | 20 | 6 | CSMD2 | chr1 | − | 34115076 | MDH2 | chr7 | + | 75525221 | 0 | 0 | 1 | 27 |
| TCGA-06-1801 | 21 | 1 | 22 | 4 | FAM192A | chr16 | − | 55757701 | LRP1 | chr12 | + | 55858598 | 0 | 0 | 1 | 26 |
| TCGA-12-1089 | 27 | 0 | 27 | 2 | FGFR4 | chr5 | + | 176447670 | LILRB1 | chr19 | + | 59840807 | 1 | 0 | 0 | 25 |
| TCGA-19-0957 | 0 | 1 | 1 | 4 | EML1 | chr14 | + | 99349006 | NRXN3 | chr14 | + | 79233969 | 1 | 0 | 0 | 24 |
| TCGA-06-1801 | 19 | 133 | 152 | 51 | NHSL2 | chrX | + | 71082676 | TAF1 | chrX | + | 70520522 | 1 | 0 | 0 | 22 |
| TCGA-06-1801 | 51 | 3 | 54 | 8 | NHSL2 | chrX | + | 71083319 | TAF1 | chrX | + | 70521607 | 1 | 0 | 1 | 22 |
| TCGA-12-1089 | 9 | 0 | 9 | 4 | CACNA1C | chr12 | + | 2325330 | ITGAV | chr2 | + | 187195411 | 0 | 0 | 1 | 22 |
| TCGA-19-0957 | 8 | 1 | 9 | 6 | CDH11 | chr16 | − | 63579650 | RERE | chr1 | − | 8588774 | 0 | 0 | 1 | 22 |
| TCGA-12-0829 | 12 | 3 | 15 | 4 | ENTPD2 | chr9 | − | 139062591 | FREM2 | chr13 | + | 38318644 | 1 | 1 | 0 | 21 |
| TCGA-12-0829 | 2 | 3 | 5 | 1 | EFS | chr14 | − | 22896776 | NRXN3 | ch14 | + | 78678529 | 1 | 0 | 1 | 21 |
| TCGA-12-0829 | 56 | 6 | 62 | 14 | DIS3L | chr15 | + | 64377566 | GLI3 | chr7 | − | 42032535 | 1 | 0 | | |
| TCGA-12-0829 | 8 | 2 | 10 | 3 | EFS | chr14 | − | 22896431 | NRXN3 | chr14 | + | 78678139 | 1 | 0 | | |
| TCGA-12-0829 | 9 | 65 | 74 | 37 | DIS3L | chr15 | + | 64377398 | GLI3 | chr7 | − | 42032341 | 1 | 0 | | |
| TCGA-27-1835 | 14 | 0 | 14 | 4 | FAM19A2 | chr12 | − | 60707200 | GLI1 | chr12 | + | 56146523 | 0 | 0 | | |
| TCGA-06-1801 | 20 | 0 | 20 | 2 | FREM2 | chr13 | + | 38163882 | RALYL | chr8 | + | 85785432 | 1 | 1 | | |
| TCGA-12-0827 | 2 | 0 | 2 | 2 | ABCC12 | chr16 | − | 46722685 | FGFR4 | chr5 | + | 176457194 | 1 | 1 | | |
| TCGA-12-0829 | 35 | 0 | 35 | 7 | ANXA7 | chr10 | − | 74808655 | CACNA1C | chr12 | + | 2458351 | 1 | 1 | | |
| TCGA-06-2559 | 60 | 37 | 97 | 1 | PLEKHM3 | chr2 | − | 208426920 | PTPRS | chr19 | − | 5222592 | 0 | 0 | | |
| TCGA-12-1088 | 2 | 0 | 2 | 2 | PLCL1 | chr2 | + | 198630224 | TACC2 | chr10 | + | 123987513 | 1 | 1 | | |
| TCGA-06-1801 | 10 | 0 | 10 | 4 | FGFR4 | chr5 | + | 176450528 | WISP2 | chr20 | + | 42782576 | 0 | 1 | | |
| TCGA-06-1802 | 15 | 0 | 15 | 2 | PDHA2 | chr4 | + | 96980717 | PDZRN4 | chr12 | + | 39959553 | 0 | 1 | | |
| TCGA-06-1802 | 4 | 0 | 4 | 2 | PDHA2 | chr4 | + | 96980509 | PDZRN4 | chr12 | + | 39959384 | 1 | 0 | | |
| TCGA-06-6390 | 53 | 0 | 53 | 18 | GPR182 | chr12 | + | 55675639 | PDZRN4 | chr12 | + | 39957003 | 1 | 0 | | |
| TCGA-12-0829 | 1121 | 252 | 1373 | 602 | ADCY8 | chr8 | − | 131886108 | SSX3 | chrX | − | 48091929 | 0 | 0 | | |
| TCGA-12-0829 | 14 | 8 | 22 | 3 | ADCY8 | chr8 | − | 131886506 | SSX3 | chrX | − | 48091719 | 1 | 1 | | |
| TCGA-12-0829 | 9 | 42 | 51 | 18 | ADAM12 | chr10 | − | 127733231 | DAPK1 | chr9 | + | 89454764 | 0 | 1 | | |
| TCGA-12-3653 | 22 | 0 | 22 | 10 | JOSD2 | chr19 | − | 55705579 | PTPRS | chr19 | − | 5245999 | 0 | 1 | | |
| TCGA-12-0829 | 100 | 0 | 100 | 20 | COL14A1 | chr8 | + | 121370990 | MMP12 | chr11 | − | 102242881 | 1 | 0 | | |
| TCGA-12-0829 | 152 | 0 | 152 | 24 | COL14A1 | chr8 | + | 121371195 | MMP12 | chr11 | − | 102242953 | 0 | 1 | | |
| TCGA-06-1802 | 11 | 47 | 58 | 19 | MUSK | chr9 | + | 112509906 | SYNPO2 | chr4 | + | 120172123 | 0 | 0 | | |
| TCGA-06-1805 | 6 | 4 | 10 | 6 | COL14A1 | chr8 | + | 121332080 | NCRNA0015 | chr21 | − | 18174873 | 1 | 1 | | |
| TCGA-12-0822 | 37 | 0 | 37 | 3 | C7orf44 | chr7 | − | 43683128 | TACC2 | chr10 | + | 123835337 | 1 | 0 | | |
| TCGA-12-0829 | 0 | 2 | 2 | 365 | GSTA3 | chr6 | − | 52878492 | TACC2 | chr10 | + | 123884543 | 0 | 1 | | |
| TCGA-12-0829 | 124 | 16 | 140 | 51 | GSTA3 | chr6 | − | 52878680 | TACC2 | chr10 | + | 123884705 | 0 | 1 | | |
| TCGA-12-0829 | 21 | 7 | 28 | 10 | HIP1 | chr7 | − | 75022909 | MASP1 | chr3 | − | 188452372 | 0 | 0 | | |
| TCGA-12-0829 | 268 | 123 | 391 | 242 | HIP1 | chr7 | − | 75022741 | MASP1 | chr3 | − | 188452581 | 1 | 1 | | |
| TCGA-12-0829 | 36 | 641 | 677 | 365 | GSTA3 | chr6 | − | 52878496 | TACC2 | chr10 | + | 123884531 | 0 | 1 | | |
| TCGA-12-1088 | 10 | 1 | 11 | 3 | CAMTA1 | chr1 | + | 7710762 | TMPRSS3 | chr21 | − | 42665918 | 0 | 1 | | |
| TCGA-12-1088 | 65 | 0 | 65 | 6 | ADCY10 | chr1 | − | 166139873 | DUSP27 | chr1 | + | 165351555 | 0 | 0 | | |
| TCGA-12-1088 | 8 | 1 | 9 | 4 | CAMTA1 | chr1 | + | 7714539 | TMPRSS3 | chr21 | − | 42666044 | 1 | 0 | | |
| TCGA-27-1835 | 83 | 1 | 84 | 22 | CMYA5 | chr5 | + | 79120729 | SRRM1 | chr1 | + | 24870899 | 0 | 0 | | |
| TCGA-06-1801 | 0 | 43 | 43 | 31 | CAMTA1 | chr1 | + | 7264935 | GDPD2 | chrX | + | 69563759 | 0 | 1 | | |
| TCGA-06-1801 | 13 | 41 | 54 | 31 | CAMTA1 | chr1 | + | 7265429 | GDPD2 | chrX | + | 69563431 | 1 | 0 | | |
| TCGA-06-1801 | 24 | 66 | 90 | 61 | CAMTA1 | chr1 | + | 7265556 | GDPD2 | chrX | + | 69563762 | 0 | 1 | | |
| TCGA-12-0829 | 2 | 0 | 2 | 3 | CCDC147 | chr10 | + | 106165013 | ISX | chr22 | + | 33795708 | 0 | 1 | | |
| TCGA-12-1088 | 7 | 1 | 8 | 5 | CMYA5 | chr5 | + | 79045621 | STK24 | chr13 | − | 97969547 | 1 | 0 | | |
| TCGA-06-1801 | 7 | 1 | 8 | 4 | DEPDC5 | chr22 | + | 30619774 | ROBO1 | chr3 | − | 79802538 | 0 | 1 | | |
| TCGA-12-0820 | 110 | 20 | 130 | 23 | ABCA13 | chr7 | + | 48597322 | NHSL2 | chrX | + | 71077547 | 1 | 0 | | |
| TCGA-12-0820 | 29 | 4 | 33 | 3 | ABCA13 | chr7 | + | 48597477 | NHSL2 | chrX | + | 71077690 | 0 | 1 | | |
| TCGA-12-0829 | 46 | 2 | 48 | 4 | LIN9 | chr1 | − | 224536835 | NCOR1 | chr17 | − | 15883585 | 0 | 0 | | |
| TCGA-12-3644 | 3 | 0 | 3 | 1 | EFHC1 | chr6 | + | 52432073 | LRBA | chr4 | − | 151418615 | 1 | 0 | | |
| TCGA-12-3644 | 3 | 10 | 13 | 3 | EFHC1 | chr6 | + | 52431890 | LRBA | chr4 | − | 151418438 | 1 | 0 | | |
| TCGA-19-5958 | 6 | 6 | 12 | 7 | DEPDC5 | chr22 | + | 30504095 | SLC5A4 | chr22 | − | 30974671 | 0 | 1 | | |
| TCGA-06-1801 | 4 | 4 | 8 | 5 | KCND3 | chr1 | − | 112227957 | LY75 | chr2 | − | 160443238 | 1 | 1 | | |
| TCGA-12-0820 | 26 | 1 | 27 | 2 | BBX | chr3 | + | 108997451 | CUL3 | chr2 | − | 225108623 | 0 | 0 | | |
| TCGA-12-0828 | 8 | 67 | 75 | 31 | ADCY2 | chr5 | + | 7558840 | SDAD1 | chr4 | − | 77096208 | 1 | 1 | | |
| TCGA-12-0829 | 13 | 21 | 34 | 16 | AGBL4 | chr1 | − | 48902776 | NUP188 | chr9 | + | 130808425 | 0 | 0 | | |
| TCGA-12-0829 | 64 | 308 | 372 | 197 | EYS | chr6 | − | 64513356 | IL1RN | chr2 | + | 113603712 | 0 | 1 | | |
| TCGA-12-0829 | 7 | 25 | 32 | 11 | AGBL4 | chr1 | − | 48902600 | NUP188 | chr9 | + | 130808628 | 1 | 1 | | |
| TCGA-12-0829 | 9 | 0 | 9 | 1 | LRBA | chr4 | − | 151790893 | PSEN1 | chr14 | + | 72707609 | 1 | 0 | | |
| TCGA-12-1093 | 65 | 4 | 69 | 21 | OSBPL10 | chr3 | − | 31687272 | TRAPPC9 | chr8 | − | 140828099 | 1 | 1 | | |
| TCGA-12-1600 | 9 | 0 | 9 | 5 | 5-Sep | chr22 | + | 18088018 | NCOR1 | chr17 | − | 15915170 | 0 | 1 | | |
| TCGA-19-0957 | 18 | 1 | 19 | 7 | ADCY10 | chr1 | − | 166060645 | AKT3 | chr1 | − | 241743142 | 0 | 1 | | |
| TCGA-19-0957 | 34 | 2 | 36 | 11 | ADCY10 | chr1 | − | 166060502 | AKT3 | chr1 | − | 241742588 | 1 | 0 | | |
| TCGA-12-0822 | 0 | 1 | 1 | 16 | ITGB2 | chr21 | − | 45147805 | SH3RF3 | chr2 | + | 109430489 | 0 | 1 | | |
| TCGA-12-0822 | 6 | 2 | 8 | 1 | ITGB2 | chr21 | − | 45147994 | SH3RF3 | chr2 | + | 109430669 | 0 | 1 | | |
| TCGA-12-0827 | 25 | 3 | 28 | 8 | CUL3 | chr2 | − | 225126210 | LY75 | chr2 | − | 160455052 | 1 | 0 | | |
| TCGA-12-0828 | 7 | 2 | 9 | 4 | FH | chr1 | − | 239743589 | SRGAP1 | chr12 | + | 62723692 | 0 | 1 | | |
| TCGA-12-0829 | 24 | 0 | 24 | 9 | ITGA9 | chr3 | + | 37712050 | SNX5 | chr20 | − | 17885523 | 0 | 1 | | |
| TCGA-12-1089 | 17 | 2 | 19 | 5 | ABCC1 | chr16 | + | 16077635 | RNF216 | chr7 | − | 5692038 | 1 | 1 | | |
| TCGA-12-1089 | 6 | 0 | 6 | 8 | CAMSAP1 | chr9 | − | 137867066 | NCF2 | chr1 | − | 181799323 | 1 | 0 | | |
| TCGA-19-0957 | 16 | 0 | 16 | 4 | CCDC147 | chr10 | + | 106114657 | STK4 | chr20 | + | 43111359 | 0 | 0 | | |
| TCGA-06-1801 | 5 | 33 | 38 | 18 | AP4S1 | chr14 | + | 30611930 | EYS | chr6 | − | 64770011 | 1 | 0 | | |
| TCGA-06-1805 | 3 | 14 | 17 | 9 | CUL3 | chr2 | − | 225064315 | SLC44A2 | chr19 | + | 10608393 | 1 | 0 | | |
| TCGA-12-0829 | 14 | 27 | 41 | 23 | ADCY2 | chr5 | + | 7798046 | C14orf174 | chr14 | + | 76914809 | 1 | 0 | | |
| TCGA-12-0829 | 59 | 7 | 66 | 18 | NR3C1 | chr5 | − | 142760085 | SORCS2 | chr4 | + | 7354165 | 0 | 0 | | |
| TCGA-12-0829 | 9 | 40 | 49 | 28 | ADCY2 | chr5 | + | 7798641 | C14orf174 | chr14 | + | 76915034 | 0 | 1 | | |
| TCGA-12-1093 | 20 | 0 | 20 | 4 | GAPVD1 | chr9 | + | 127104266 | MAPKAP1 | chr9 | − | 127490362 | 1 | 0 | | |
| TCGA-12-1600 | 7 | 0 | 7 | 4 | CILP | chr15 | − | 63283865 | PARP16 | chr15 | − | 63350048 | 0 | 1 | 1 | 10 |
| TCGA-19-0957 | 13 | 3 | 16 | 6 | AQP2 | chr12 | + | 48635567 | CDH4 | chr20 | + | 59413648 | 0 | 1 | 1 | 10 |
| TCGA-19-0957 | 6 | 0 | 6 | 1 | AQP2 | chr12 | + | 48635406 | CDH4 | chr20 | + | 59413468 | 1 | 0 | 0 | 10 |
| TCGA-06-0166 | 2 | 0 | 2 | 3 | CCDC158 | chr4 | − | 77541796 | SNX5 | chr20 | − | 17885346 | 0 | 1 | 1 | 9 |
| TCGA-06-1802 | 30 | 0 | 30 | 9 | RANBP2 | chr2 | + | 108758804 | SATB2 | chr2 | − | 199895572 | 0 | 1 | 1 | 9 |
| TCGA-06-1805 | 4 | 0 | 4 | 3 | C2CD3 | chr11 | − | 73430819 | XRRA1 | chr11 | − | 74309669 | 1 | 1 | 0 | 9 |
| TCGA-06-1805 | 6 | 1 | 7 | 5 | NEUROG1 | chr5 | − | 134898853 | PRKCH | chr14 | + | 51027580 | 1 | 1 | 0 | 9 |
| TCGA-12-0820 | 27 | 0 | 27 | 2 | RANBP2 | chr2 | + | 108749908 | TTC27 | chr2 | + | 32839367 | 0 | 1 | 1 | 9 |
| TCGA-12-0820 | 58 | 7 | 65 | 15 | RANBP2 | chr2 | + | 108749412 | TTC27 | chr2 | + | 32837790 | 1 | 0 | 0 | 9 |
| TCGA-12-0829 | 6 | 128 | 134 | 35 | C2CD3 | chr11 | − | 73529639 | CAPZB | chr1 | − | 19556435 | 0 | 0 | 1 | 9 |
| TCGA-12-0829 | 84 | 443 | 527 | 227 | C2CD3 | chr11 | − | 73529293 | CAPZB | chr1 | − | 19556627 | 1 | 1 | 0 | 9 |
| TCGA-12-1088 | 10 | 0 | 10 | 2 | PACSIN1 | chr6 | + | 34589431 | TNC | chr9 | − | 116884742 | 0 | 1 | 0 | 9 |
| TCGA-12-1088 | 12 | 0 | 12 | 2 | PACSIN1 | chr6 | + | 34589619 | TNC | chr9 | − | 116884958 | 1 | 0 | 1 | 9 |
| TCGA-19-0957 | 34 | 19 | 53 | 17 | PRKCH | chr14 | + | 61032978 | ZFAND3 | chr6 | + | 38228111 | 1 | 0 | 1 | 9 |
| TCGA-19-0957 | 7 | 1 | 8 | 7 | MAPKAP1 | chr9 | − | 127348507 | SLC9A1 | chr1 | − | 27302334 | 0 | 1 | 0 | 9 |
| TCGA-19-0957 | 8 | 39 | 47 | 21 | PRKCH | chr14 | + | 61032774 | ZFAND3 | chr6 | + | 38227949 | 1 | 0 | 0 | 9 |
| TCGA-06-1801 | 5 | 11 | 16 | 4 | MAOA | chrX | + | 43486192 | SH3RF3 | chr2 | + | 109237058 | 1 | 0 | 1 | 8 |
| TCGA-06-1802 | 10 | 12 | 22 | 12 | DNM1L | chr12 | + | 32736794 | SYNPO2 | chr4 | + | 120172271 | 0 | 1 | 1 | 8 |
| TCGA-06-1802 | 18 | 42 | 60 | 24 | MUC4 | chr3 | − | 196982875 | SMOC2 | chr6 | + | 168676813 | 1 | 0 | 1 | 8 |
| TCGA-12-0829 | 6 | 0 | 6 | 6 | ATXN1 | chr6 | − | 16669201 | CACNA1G | chr17 | + | 46004995 | 0 | 0 | 1 | 8 |
| TCGA-12-0829 | 7 | 0 | 7 | 3 | ATP6V0D2 | chr8 | + | 87186716 | RERE | chr1 | − | 8336574 | 1 | 1 | 0 | 8 |
| TCGA-12-1088 | 11 | 1 | 12 | 6 | BCAS3 | chr17 | + | 56321892 | CACNA1G | chr17 | + | 46010698 | 1 | 1 | 0 | 8 |
| TCGA-12-1088 | 15 | 2 | 17 | 5 | ABCC1 | chr16 | + | 16135771 | AGBL4 | chr1 | − | 49315120 | 1 | 0 | 0 | 8 |
| TCGA-12-1088 | 17 | 3 | 20 | 4 | MST1R | chr3 | − | 49910627 | WDFY1 | chr2 | − | 224512774 | 1 | 0 | 0 | 8 |
| TCGA-12-1088 | 4 | 0 | 4 | 2 | FBXL4 | chr6 | − | 99431443 | SYNPO2 | chr4 | + | 120172560 | 0 | 0 | 1 | 8 |
| TCGA-12-1092 | 39 | 4 | 43 | 12 | CNTN2 | chr1 | + | 203302926 | DNAJC6 | chr1 | + | 65591195 | 0 | 0 | 1 | 8 |
| TCGA-12-1598 | 4 | 0 | 4 | 5 | MPP1 | chrX | − | 153673715 | SRGAP1 | chr12 | + | 62777947 | 0 | 0 | 1 | 8 |
| TCGA-19-1786 | 5 | 19 | 24 | 7 | ATP5B | chr12 | − | 55320148 | USP48 | chr1 | − | 21920103 | 1 | 0 | 1 | 8 |
| TCGA-19-2621 | 21 | 0 | 21 | 3 | BCAS3 | chr17 | + | 56731673 | TTYH1 | chr19 | + | 59638801 | 1 | 1 | 0 | 8 |
| TCGA-06-1801 | 15 | 0 | 15 | 9 | C15orf23 | chr15 | + | 38469150 | DMD | chrX | − | 32092185 | 0 | 1 | 1 | 7 |
| TCGA-06-1805 | 6 | 3 | 9 | 5 | FAM19A2 | chr12 | − | 60547321 | POLM | chr7 | − | 44082653 | 0 | 1 | 0 | 7 |
| TCGA-12-0829 | 13 | 84 | 97 | 44 | ATP5B | chr12 | − | 55318484 | PRC1 | chr15 | − | 89330475 | 1 | 0 | 1 | 7 |
| TCGA-12-0829 | 158 | 207 | 365 | 44 | ATP5B | chr12 | − | 55320850 | PRC1 | chr15 | − | 89334458 | 1 | 1 | 0 | 7 |
| TCGA-12-0829 | 2 | 1 | 3 | 2 | DDI2 | chr1 | + | 15825507 | KIDINS220 | chr2 | − | 8805399 | 0 | 1 | 0 | 7 |
| TCGA-12-0829 | 25 | 6 | 31 | 44 | ATP5B | chr12 | − | 55321832 | PRC1 | chr15 | − | 89335627 | 1 | 0 | 1 | 7 |
| TCGA-12-0829 | 34 | 21 | 55 | 44 | ATP5B | chr12 | − | 55321200 | PRC1 | chr15 | − | 89335044 | 0 | 1 | 0 | 7 |
| TCGA-12-0829 | 44 | 6 | 50 | 4 | ABCC6 | chr16 | − | 16204784 | SUMF1 | chr3 | − | 4470138 | 1 | 0 | 0 | 7 |
| TCGA-12-0829 | 53 | 28 | 81 | 35 | DDI2 | chr1 | + | 15825941 | KIDINS220 | chr2 | − | 8805580 | 1 | 0 | 1 | 7 |
| TCGA-12-0829 | 9 | 0 | 9 | 5 | DMD | chrX | − | 32013100 | N4BP2L2 | chr13 | − | 32008512 | 0 | 0 | 1 | 7 |
| TCGA-12-1092 | 9 | 0 | 9 | 4 | LRRC48 | chr19 | − | 55754780 | NR3C1 | chr5 | − | 142660156 | 0 | 1 | 0 | 7 |
| TCGA-19-2621 | 3 | 22 | 25 | 11 | PCDH12 | chr5 | − | 141309153 | SLC36A2 | chr5 | − | 150679274 | 1 | 1 | 0 | 7 |
| TCGA-06-1802 | 8 | 4 | 12 | 6 | BAHD1 | chr15 | + | 38539023 | OSBPL10 | chr3 | − | 31729622 | 0 | 1 | 1 | 6 |
| TCGA-12-0828 | 11 | 0 | 11 | 1 | PLOD3 | chr7 | − | 100646340 | VSNL1 | chr2 | + | 17638618 | 1 | 1 | 0 | 6 |
| TCGA-12-0828 | 40 | 9 | 49 | 20 | PLOD3 | chr7 | − | 100646511 | VSNL1 | chr2 | + | 17637955 | 0 | 0 | 1 | 6 |
| TCGA-12-0829 | 16 | 2 | 18 | 9 | C21orf29 | chr21 | − | 44922864 | MYT1 | chr20 | + | 62300829 | 0 | 0 | 1 | 6 |
| TCGA-12-0829 | 196 | 0 | 196 | 37 | IGFBP3 | chr7 | − | 45922866 | SMOC2 | chr6 | + | 168722450 | 0 | 0 | 1 | 6 |
| TCGA-12-0829 | 5 | 1 | 6 | 1 | FAM168A | chr11 | − | 72839771 | NCF2 | chr1 | − | 181826115 | 1 | 0 | 1 | 6 |
| TCGA-12-0829 | 5 | 18 | 23 | 9 | FAM168A | chr11 | − | 72839534 | NCF2 | chr1 | − | 181825930 | 1 | 0 | 0 | 6 |
| TCGA-12-1089 | 20 | 0 | 20 | 2 | SLC44A2 | chr19 | + | 10602997 | XRCC4 | chr5 | + | 82430803 | 1 | 0 | 0 | 6 |
| TCGA-19-0957 | 17 | 1 | 18 | 4 | PAX3 | chr2 | − | 222778052 | WDFY1 | chr2 | − | 224453159 | 1 | 0 | 1 | 6 |
| TCGA-06-1801 | 5 | 0 | 5 | 1 | CAP2 | chr6 | + | 17571234 | DNAJC6 | chr1 | + | 65602700 | 1 | 0 | 0 | 5 |
| TCGA-06-1801 | 6 | 32 | 38 | 15 | CAP2 | chr6 | + | 17571666 | DNAJC6 | chr1 | + | 65603089 | 0 | 1 | 1 | 5 |
| TCGA-06-1805 | 3 | 0 | 3 | 8 | PLCL1 | chr2 | + | 198578552 | SURF6 | chr9 | − | 135188818 | 0 | 1 | 0 | 5 |
| TCGA-06-1805 | 7 | 2 | 9 | 4 | PLCL1 | chr2 | + | 198578671 | SURF6 | chr9 | − | 135189294 | 1 | 0 | 1 | 5 |
| TCGA-12-0822 | 17 | 4 | 21 | 4 | TAAR6 | chr6 | + | 132933266 | TTYH1 | chr19 | + | 59629451 | 0 | 1 | 1 | 5 |
| TCGA-12-0828 | 17 | 0 | 17 | 8 | AQP2 | chr12 | + | 48634800 | ECE1 | chr1 | − | 21515240 | 0 | 1 | 1 | 5 |
| TCGA-12-0828 | 7 | 0 | 7 | 1 | AQP2 | chr12 | + | 48634610 | ECE1 | chr1 | − | 21515033 | 1 | 0 | 0 | 5 |
| TCGA-12-0829 | 12 | 0 | 12 | 7 | CACNA1G | chr17 | + | 46039372 | CNTNAP4 | chr16 | + | 74873868 | 0 | 1 | 1 | 5 |
| TCGA-19-0957 | 4 | 0 | 4 | 3 | PCDH12 | chr5 | − | 141316624 | SH3BP5 | chr3 | − | 15315567 | 0 | 0 | 1 | 5 |
| TCGA-19-0957 | 8 | 2 | 10 | 2 | PCDH12 | chr5 | − | 141316405 | SH3BP5 | chr3 | − | 15315731 | 1 | 1 | 0 | 5 |
| TCGA-06-1801 | 13 | 1 | 14 | 1 | ABCC6 | chr16 | − | 16205051 | CMTM7 | chr3 | + | 32443880 | 0 | 1 | 1 | 4 |
| TCGA-06-1801 | 33 | 7 | 40 | 4 | ABCC6 | chr16 | − | 16204860 | CMTM7 | chr3 | + | 32443722 | 1 | 0 | 0 | 4 |
| TCGA-06-1805 | 10 | 3 | 13 | 6 | AGBL4 | chr1 | − | 49449813 | NOX4 | chr11 | − | 88714996 | 0 | 0 | 1 | 4 |
| TCGA-12-0829 | 11 | 0 | 11 | 4 | FAM160A1 | chr4 | + | 152595916 | LY75 | chr2 | − | 160440194 | 0 | 1 | 0 | 4 |
| TCGA-12-0829 | 17 | 1 | 18 | 5 | FAM160A1 | chr4 | + | 152596097 | LY75 | chr2 | − | 160440376 | 1 | 0 | 1 | 4 |
| TCGA-12-0829 | 487 | 83 | 570 | 249 | CORO7 | chr16 | − | 4375428 | DYRK3 | chr1 | + | 204876154 | 0 | 0 | 1 | 4 |
| TCGA-12-1088 | 2 | 12 | 14 | 4 | FAM172A | chr5 | − | 93052315 | TRIOBP | chr22 | + | 36427382 | 0 | 1 | 1 | 4 |
| TCGA-06-1801 | 30 | 7 | 37 | 18 | DEPDC7 | chr11 | + | 33003811 | EIF2C2 | chr8 | − | 141618836 | 1 | 0 | 0 | 3 |
| TCGA-06-1801 | 40 | 28 | 68 | 33 | MAP7 | chr6 | − | 136728609 | SH3RF3 | chr2 | + | 109392677 | 0 | 0 | | |
| TCGA-12-1093 | 6 | 15 | 21 | 8 | CORO7 | chr16 | − | 4398302 | PLEK2 | chr14 | − | 66934201 | 0 | 1 | | |
| TCGA-12-3644 | 33 | 0 | 33 | 4 | EDA | chrX | + | 69073054 | SSX3 | chrX | − | 48094443 | 1 | 1 | | |
| TCGA-12-3644 | 37 | 15 | 52 | 17 | C15orf33 | chr15 | − | 47424122 | PARP16 | chr15 | − | 63350289 | 0 | 0 | | |
| TCGA-19-1791 | 14 | 3 | 17 | 8 | PSEN1 | chr14 | + | 72748293 | ZNF410 | chr14 | + | 73431112 | 0 | 0 | | |
| TCGA-06-1802 | 35 | 2 | 37 | 17 | CELF2 | chr10 | + | 11352537 | PLA2G2F | chr1 | + | 20348173 | 1 | 1 | | |
| TCGA-06-1802 | 63 | 26 | 89 | 25 | CELF2 | chr10 | + | 11352765 | PLA2G2F | chr1 | + | 20347997 | 0 | 0 | | |
| TCGA-06-1802 | 8 | 2 | 10 | 8 | LCLAT1 | chr2 | + | 30535977 | PACSIN1 | chr6 | + | 34576195 | 1 | 0 | | |
| TCGA-06-2562 | 6 | 0 | 6 | 4 | SNTA1 | chr20 | − | 31473415 | TMEM80 | chr11 | + | 689744 | 0 | 0 | | |
| TCGA-12-0829 | 16 | 1 | 17 | 4 | LASS6 | chr2 | + | 169045211 | NKAIN2 | chr6 | + | 125021252 | 0 | 0 | | |
| TCGA-12-0829 | 7 | 0 | 7 | 2 | LASS6 | chr2 | + | 169045333 | NKAIN2 | chr6 | + | 125021072 | 1 | 1 | | |
| TCGA-14-0813 | 339 | 39 | 378 | 5 | SNTA1 | chr20 | − | 31481069 | TMEM80 | chr11 | + | 686739 | 0 | 0 | | |
| TCGA-12-0820 | 49 | 3 | 52 | 11 | CAMKK1 | chr17 | − | 3712344 | FAM184B | chr4 | − | 17271273 | 0 | 1 | | |
| TCGA-12-0826 | 8 | 18 | 26 | 13 | CELF2 | chr10 | + | 11406463 | NME4 | chr16 | + | 389427 | 1 | 1 | | |
| TCGA-12-1089 | 17 | 4 | 21 | 10 | C6orf170 | chr6 | − | 121478035 | NKAIN2 | chr6 | + | 125083380 | 1 | 0 | | |
| TCGA-12-1600 | 19 | 0 | 19 | 3 | ATP6AP1L | chr5 | + | 81649744 | FAM172A | chr5 | − | 93336459 | 1 | 0 | | |
| TCGA-12-1600 | 5 | 35 | 40 | 6 | ATP6AP1L | chr5 | + | 81649902 | FAM172A | chr5 | − | 93336676 | 1 | 0 | | |
| TCGA-19-1790 | 4 | 0 | 4 | 4 | ARMC6 | chr19 | + | 19026932 | FAM184B | chr4 | − | 17391210 | 1 | 0 | | |
| TCGA-06-1802 | 12 | 0 | 12 | 8 | EIF2C2 | chr8 | − | 141648334 | TNFRSF10B | chr8 | − | 22940680 | 0 | 0 | | |
| TCGA-14-0781 | 22 | 2 | 24 | 8 | FAM160A1 | chr4 | + | 152584637 | UNC93B1 | chr11 | − | 67523253 | 1 | 0 | | |

TABLE 3
Recurrent gene fusion pairs from Exome-
fuse analysis of 84 GBM from TCGA.

| Sample | gene A | gene B |
|---|---|---|
| TCGA-12-0820 | ABCA13 | NHSL2 |
| TCGA-12-1089 | ABCC1 | RNF216 |
| TCGA-12-1088 | ABCC1 | AGBL4 |
| TCGA-12-0827 | ABCC12 | FGFR4 |
| TCGA-12-0829 | ABCC6 | SUMF1 |
| TCGA-06-1801 | ABCC6 | CMTM7 |
| TCGA-12-1088 | ABL1 | TNFRSF10B |
| TCGA-06-1802 | ADAM12 | PTPRD |
| TCGA-12-0829 | ADAM12 | DAPK1 |
| TCGA-12-1088 | ADCY10 | DUSP27 |
| TCGA-19-0957 | ADCY10 | AKT3 |
| TCGA-12-0828 | ADCY2 | SDAD1 |
| TCGA-12-0829 | ADCY2 | C14orf174 |
| TCGA-12-0829 | ADCY8 | SSX3 |
| TCGA-12-0829 | AGBL4 | NUP188 |
| TCGA-06-1805 | AGBL4 | NOX4 |
| TCGA-12-1089 | AHNAK | C21orf29 |
| TCGA-12-0829 | ANXA7 | CACNA1C |
| TCGA-06-1801 | AP4S1 | EYS |
| TCGA-12-0828 | AQP2 | ECE1 |
| TCGA-19-0957 | AQP2 | CDH4 |
| TCGA-19-1790 | ARMC6 | FAM184B |
| TCGA-19-1786 | ATP5B | USP48 |
| TCGA-12-0829 | ATP5B | PRC1 |
| TCGA-12-1600 | ATP6AP1L | FAM172A |
| TCGA-12-0829 | ATP6V0D2 | RERE |
| TCGA-12-0829 | ATXN1 | CACNA1G |
| TCGA-06-1802 | BAHD1 | OSBPL10 |
| TCGA-12-0820 | BBX | CUL3 |
| TCGA-19-2621 | BCAS3 | TTYH1 |
| TCGA-12-1088 | BCAS3 | CACNA1G |
| TCGA-06-1801 | C15orf23 | DMD |
| TCGA-12-3644 | C15orf33 | PARP16 |
| TCGA-12-0829 | C21orf29 | MYT1 |
| TCGA-06-1805 | C2CD3 | XRRA1 |
| TCGA-12-0829 | C2CD3 | CAPZ8 |
| TCGA-12-1089 | C6orf170 | NKAIN2 |
| TCGA-12-0822 | C7orf44 | TACC2 |
| TCGA-12-1089 | CACNA1C | ITGAV |
| TCGA-12-0829 | CACNA1G | CNTNAP4 |
| TCGA-12-0820 | CAMKK1 | FAM184B |
| TCGA-12-1089 | CAMSAP1 | NCF2 |
| TCGA-12-1088 | CAMTA1 | TMPRSS3 |
| TCGA-06-1801 | CAMTA1 | GDPD2 |
| TCGA-06-1801 | CAP2 | DNAJC6 |
| TCGA-19-0957 | CCDC147 | STK4 |
| TCGA-12-0829 | CCDC147 | ISX |
| TCGA-06-0166 | CCDC158 | SNX5 |
| TCGA-19-0957 | CDH11 | RERE |
| TCGA-06-1802 | CELF2 | PLA2G2F |
| TCGA-12-0826 | CELF2 | NME4 |
| TCGA-12-1600 | CILP | PARP16 |
| TCGA-12-1089 | CLK3 | LRP1 |
| TCGA-12-1088 | CMYA5 | STK24 |
| TCGA-27-1835 | CMYA5 | SRRM1 |
| TCGA-12-1092 | CNTN2 | DNAJC6 |
| TCGA-06-1805 | COL14A1 | NCRNA00157 |
| TCGA-12-0829 | COL14A1 | MMP12 |
| TCGA-12-1093 | CORO7 | PLEK2 |
| TCGA-12-0829 | CORO7 | DYRK3 |
| TCGA-06-1801 | CROCC | CSMD2 |
| TCGA-19-0957 | CSMD2 | MDH2 |
| TCGA-06-1805 | CUL3 | SLC44A2 |
| TCGA-12-0827 | CUL3 | LY75 |
| TCGA-12-0829 | DDI2 | KIDINS220 |
| TCGA-19-5958 | DEPDC5 | SLC5A4 |
| TCGA-06-1801 | DEPDC5 | ROBO1 |
| TCGA-06-1801 | DEPDC7 | EIF2C2 |
| TCGA-12-0829 | DIS3L | GLI3 |
| TCGA-12-0829 | DMD | N4BP2L2 |
| TCGA-06-1802 | DNM1L | SYNPO2 |
| TCGA-12-3644 | EDA | SSX3 |
| TCGA-12-3644 | EFHC1 | LRBA |
| TCGA-12-0829 | EFS | NRXN3 |
| TCGA-06-1802 | EIF2C2 | TNFRSF10B |
| TCGA-19-0957 | EML1 | NRXN3 |
| TCGA-12-0829 | ENTPD2 | FREM2 |
| TCGA-12-0829 | EYS | IL1RN |
| TCGA-14-0781 | FAM160A1 | UNC93B1 |
| TCGA-12-0829 | FAM160A1 | LY75 |

Table 3 above shows recurrent gene fusion pairs from Exome-fuse analysis of 84 GBM from TCGA. Fusion candidates have been nominated if they have at least two split inserts and at least two split reads. To further filter the list on recurrence, any fusion candidate was kept in which one of the genes is involved in at least two fusions across different samples.

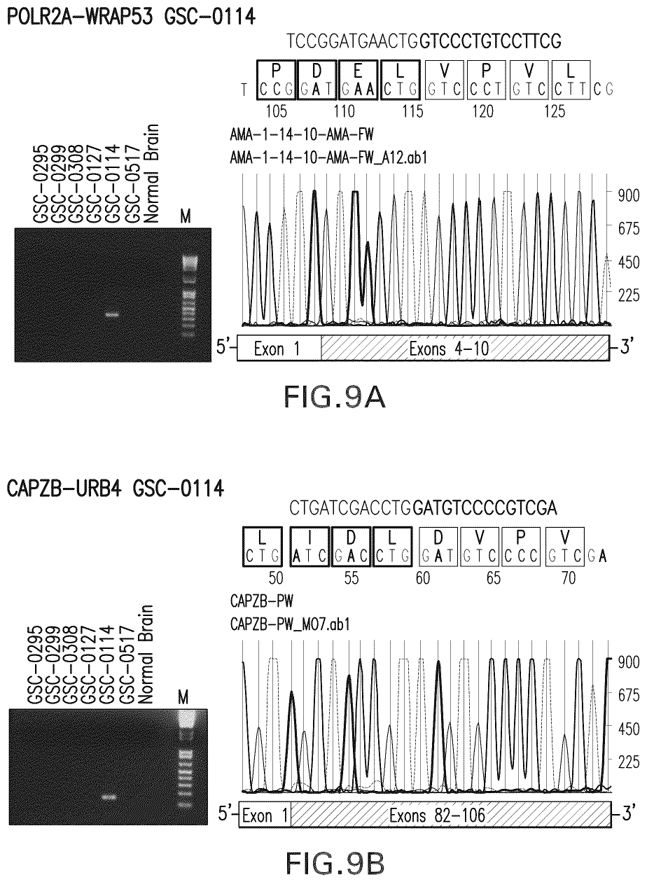

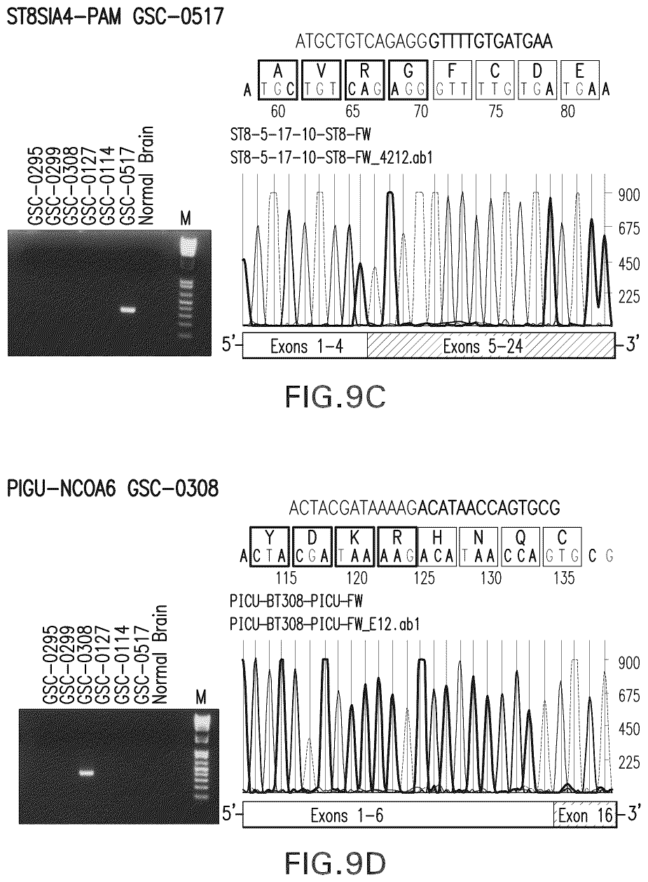

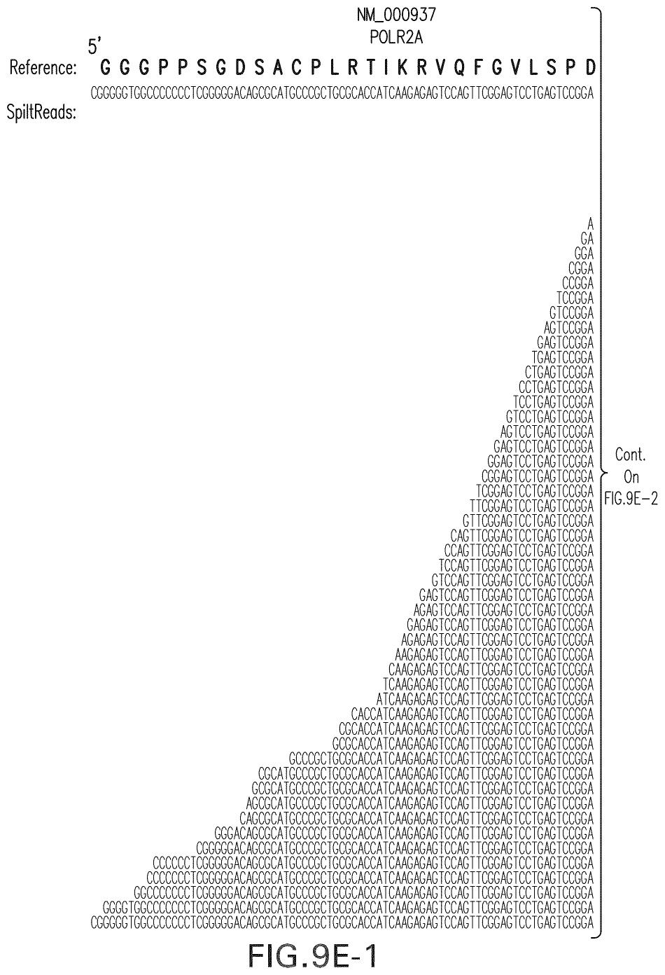

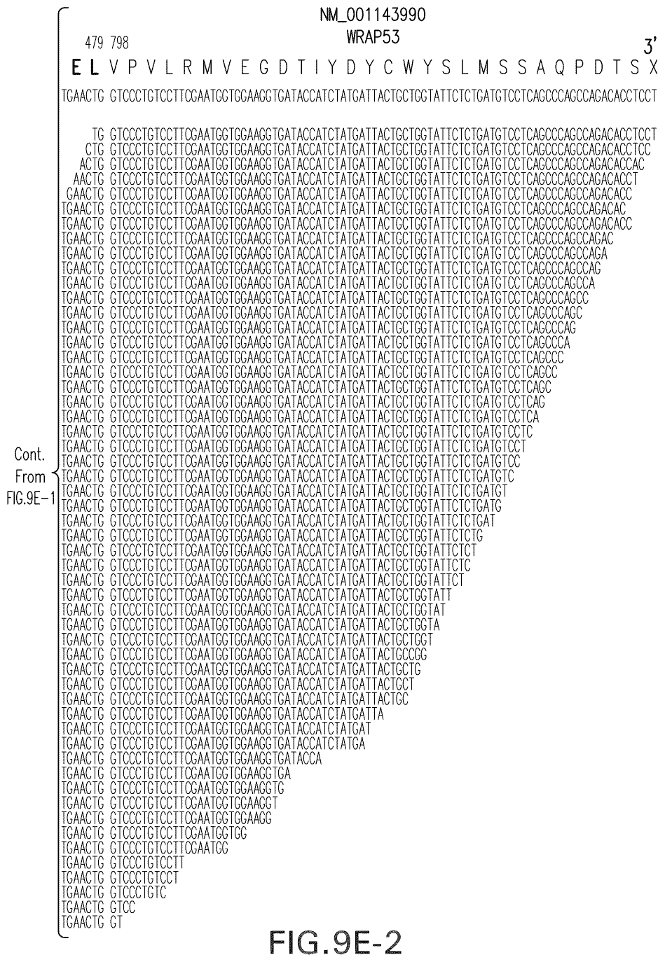

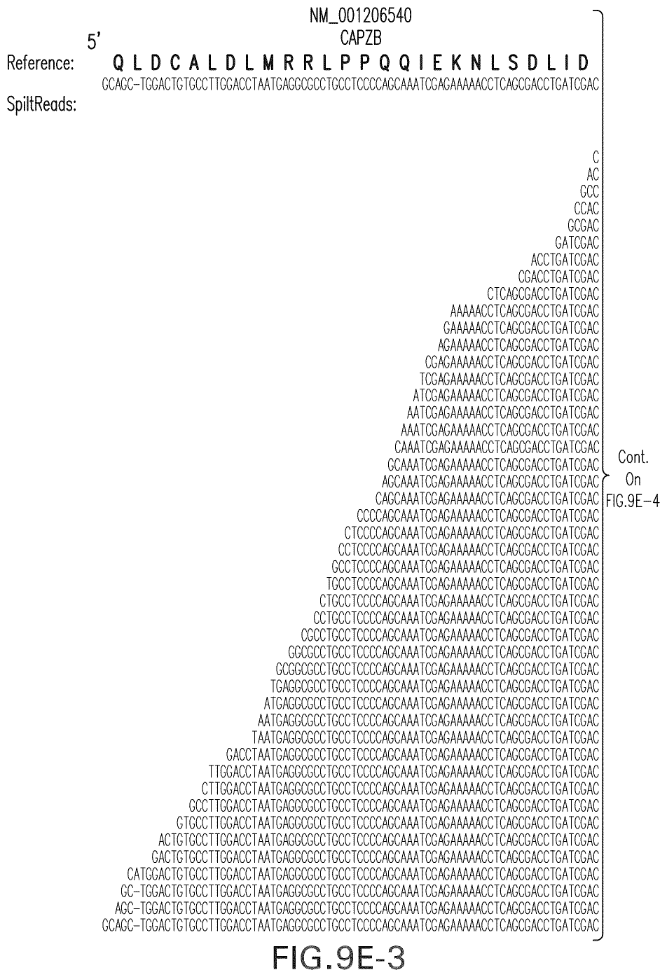

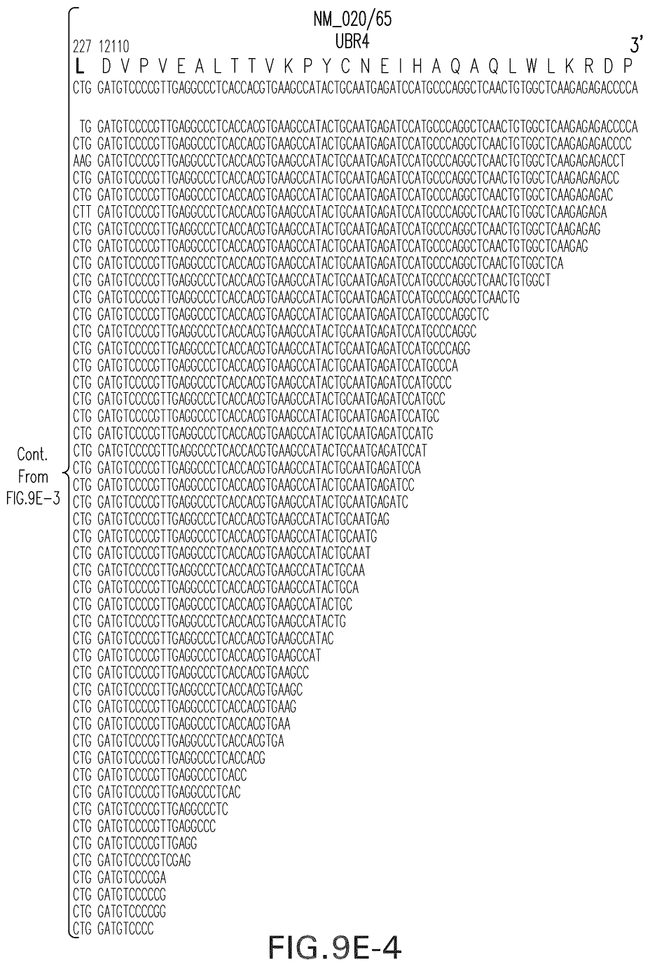

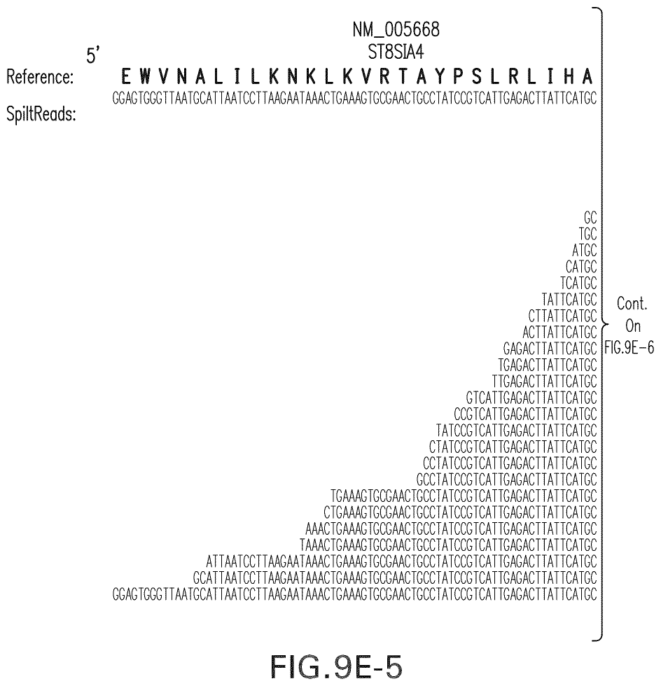

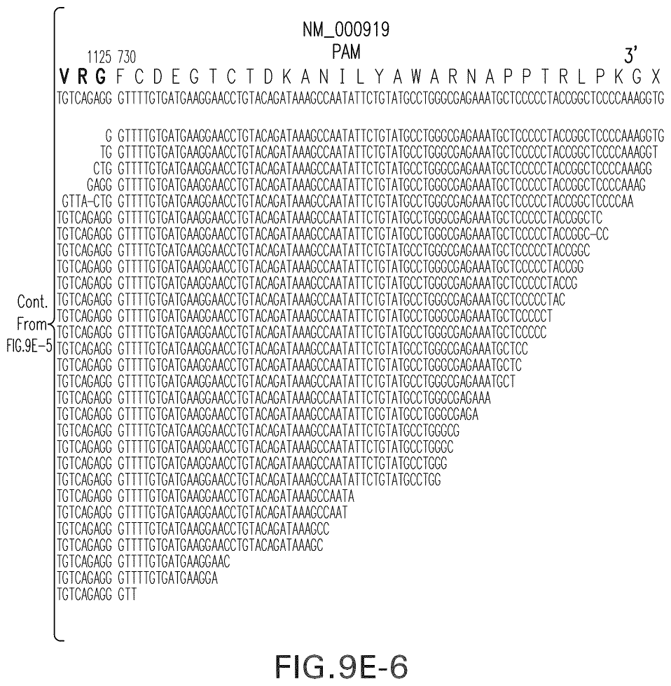

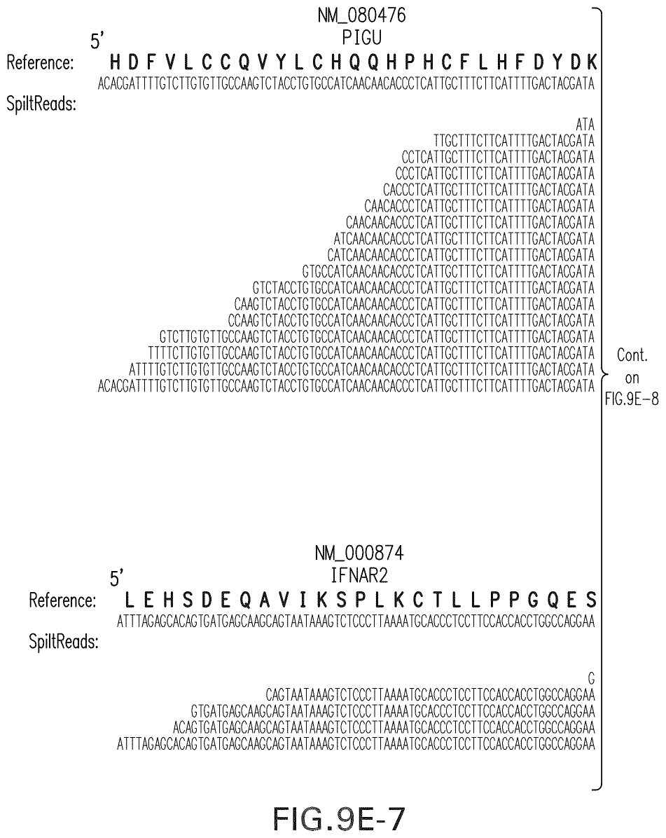

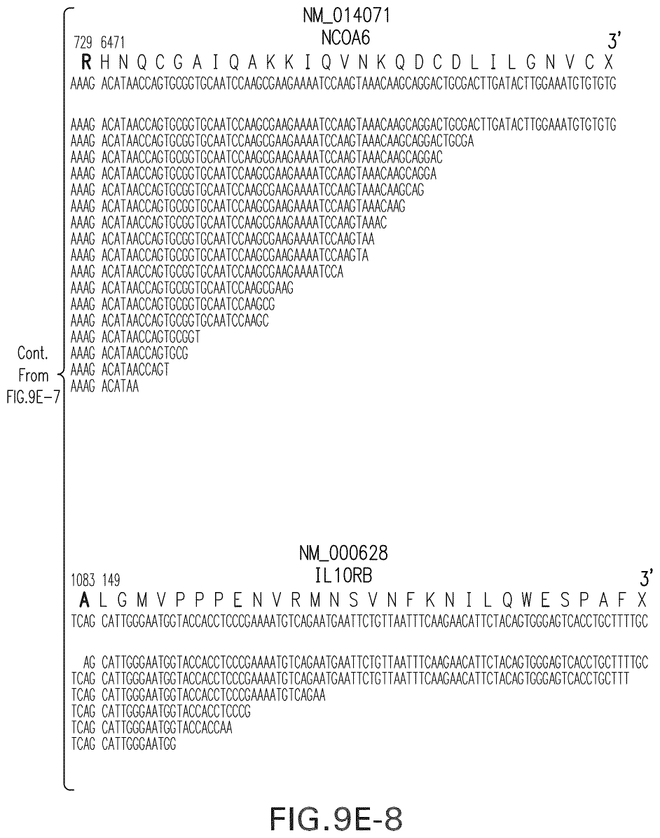

To experimentally validate the computational predictions that emerged from TX-Fuse, the PCR products spanning the fusion breakpoint were sequenced and validated each of the five in-frame fusion predictions (FIGS. 1 and 9). In FIG. 1B, the prediction is shown and in FIG. 1C, the cDNA sequence validation for the fusion with the highest read support involving FGFR3 fused in-frame with TACC3 in GSC-1123 is shown. The same FGFR3-TACC3 fusion transcript was also detected in the primary GBM-1123 tumor specimen from which the GSC-1123 culture was established (FIG. 1C). The amplified cDNA contained an open reading frame for a protein of 1,048 amino acids resulting from the fusion of a FGFR3 amino-terminal portion of residues 1-758 with a TACC3 carboxy-terminal portion of residues 549-838 (FIG. 1D). FGFR3 is a member of the FGFR receptor tyrosine kinase (TK) family that transduces intracellular signals after binding to FGF ligands (Turner and Grose, 2010). TACC3 belongs to the evolutionarily conserved TACC gene family, which also includes TACC1 and TACC2. The distinctive feature of TACC proteins is the presence of a coiled-coil domain at the C-terminus, known as the TACC domain. Through the TACC domain, TACC proteins localize to the mitotic spindle during metaphase and stabilize the microtubule spindle network (Hood and Royle, 2011; Peset and Vernos, 2008). In the predicted fusion protein the intracellular TK domain of FGFR3 is fused upstream of the TACC domain of TACC3 (FIG. 1D).

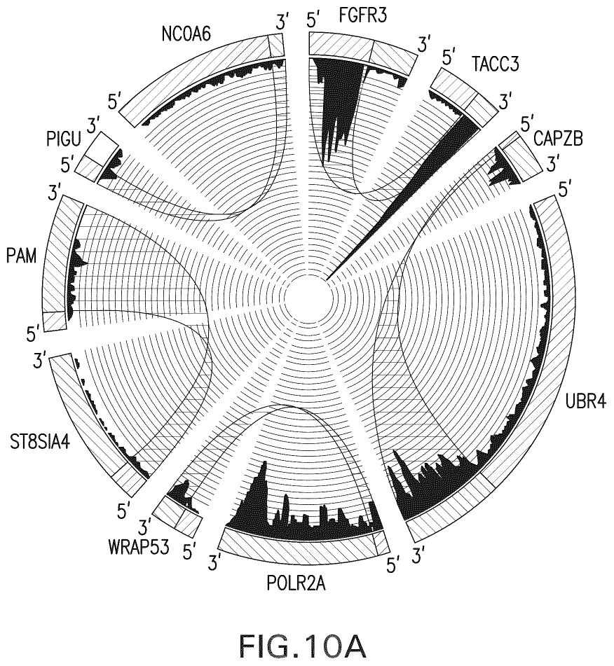

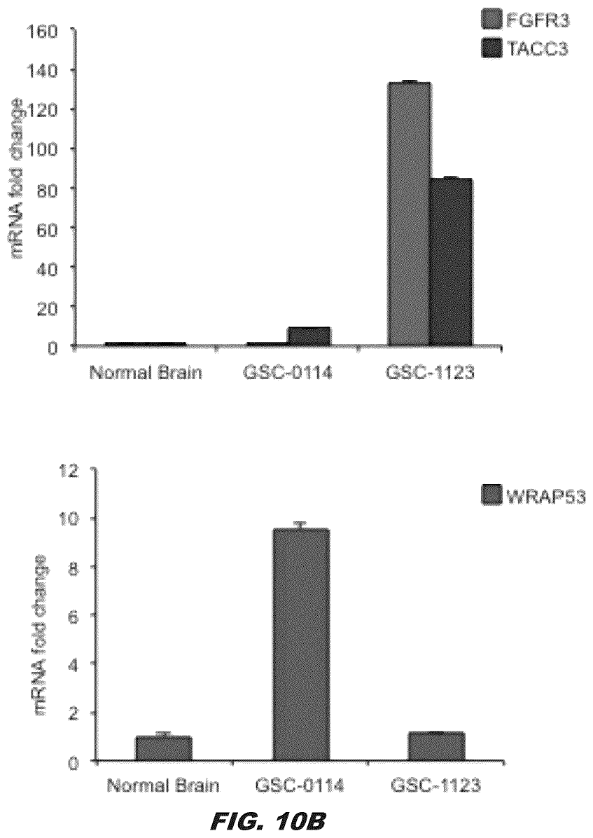

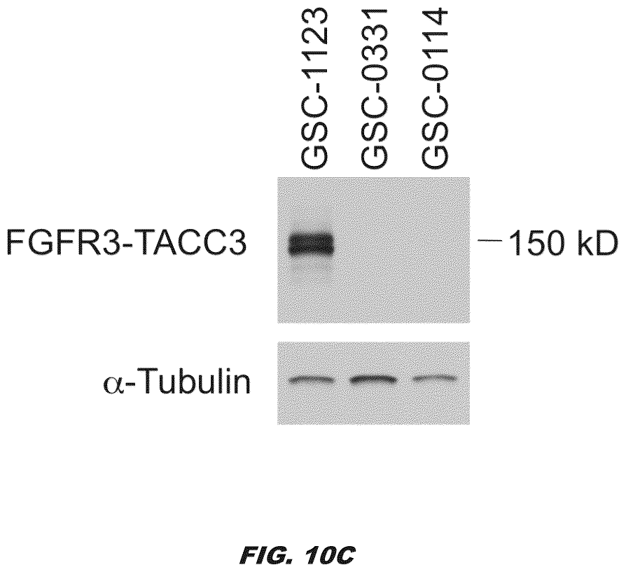

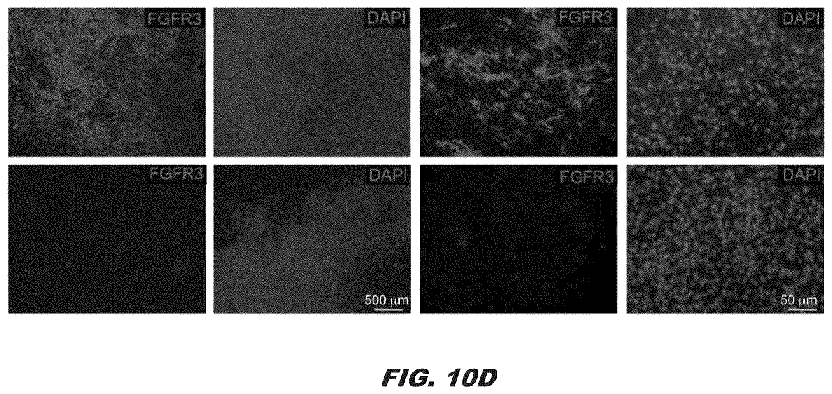

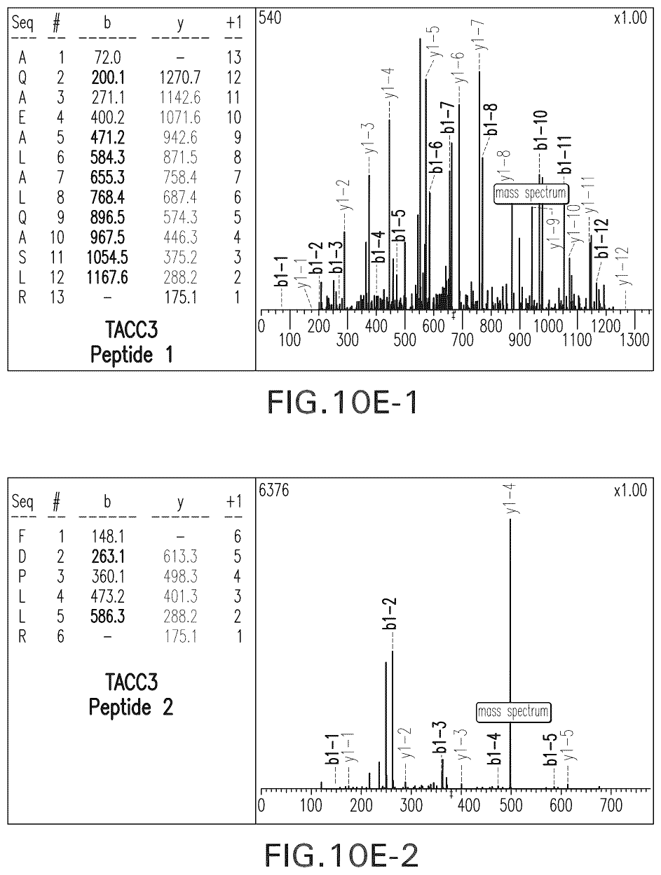

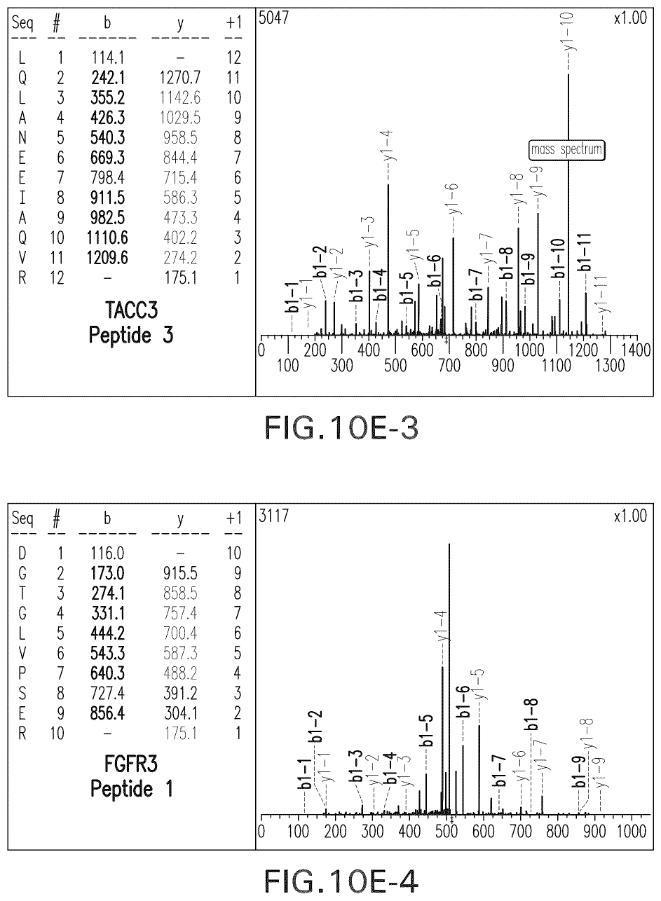

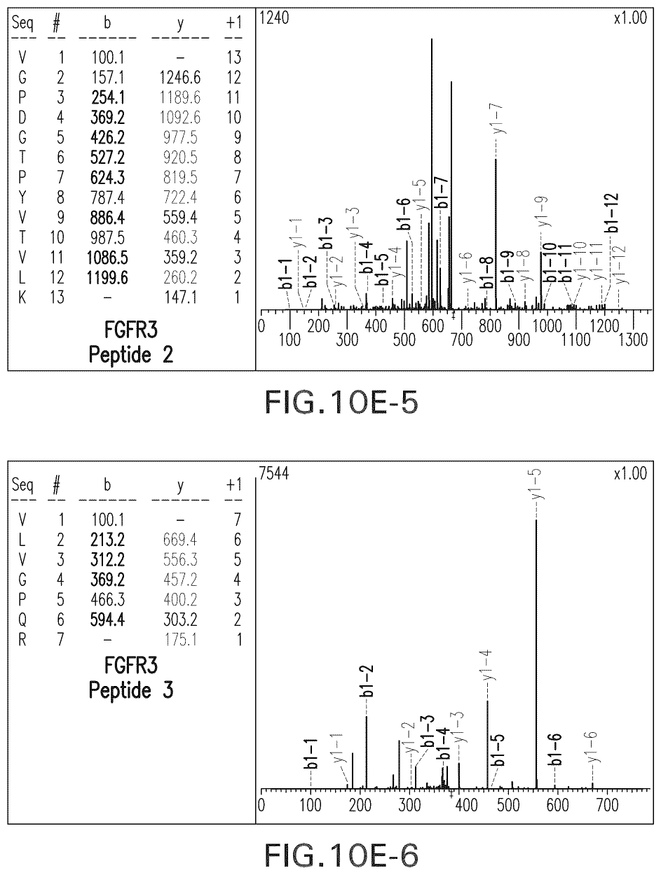

Exon-specific gene expression analysis from the RNA-seq coverage in GSC-1123 demonstrated that the FGFR3 and TACC3 exons implicated in the fusion are highly overexpressed compared with the mRNA sequences not included in the fusion event (FIG. 10A). Quantitative RT-PCR showed that the expression of the fused FGFR3-TACC3 exons is significantly higher in GSC-1123 than other GSCs and the normal brain (80 to 130-fold, FIG. 10B). Without being bound by theory, functionally significant genetic rearrangements may result in marked overexpression (outlier) of the genes implicated in the fusion events (Tomlins et al., 2007; Tomlins et al., 2005). The FGFR3-TACC3 fusion protein was also abundantly expressed in GSC-1123 and in the primary tumor GBM-1123, as shown by Western blot and immunohistochemistry (FIGS. 10C and 10D). On a Western Blot, the FGFR3-TACC3 fusion protein migrated at a size of ˜150 kD and immunoprecipitation followed by mass spectrometry revealed the presence of FGFR3 and TACC3 peptides consistent with the cDNA translation prediction (FIG. 10E). Using PCR, the genomic breakpoint coordinates were mapped to chromosome 4 (#1,808,966 for FGFR3 and #1,737,080 for TACC3, genome build GRCh37/hg19) falling within FGFR3 exon 17 and TACC3 intron 7, which gives rise to a transcript in which the 5′ FGFR3 exon 16 is spliced to the 3′ TACC3 exon 8. The DNA junctions of FGFR3 and TACC3 show microhomology within a 10-base region, an observation consistent with results previously reported for other chromosomal rearrangements in human cancer (Bass et al., 2011; Stephens et al., 2009) (FIG. 1E).

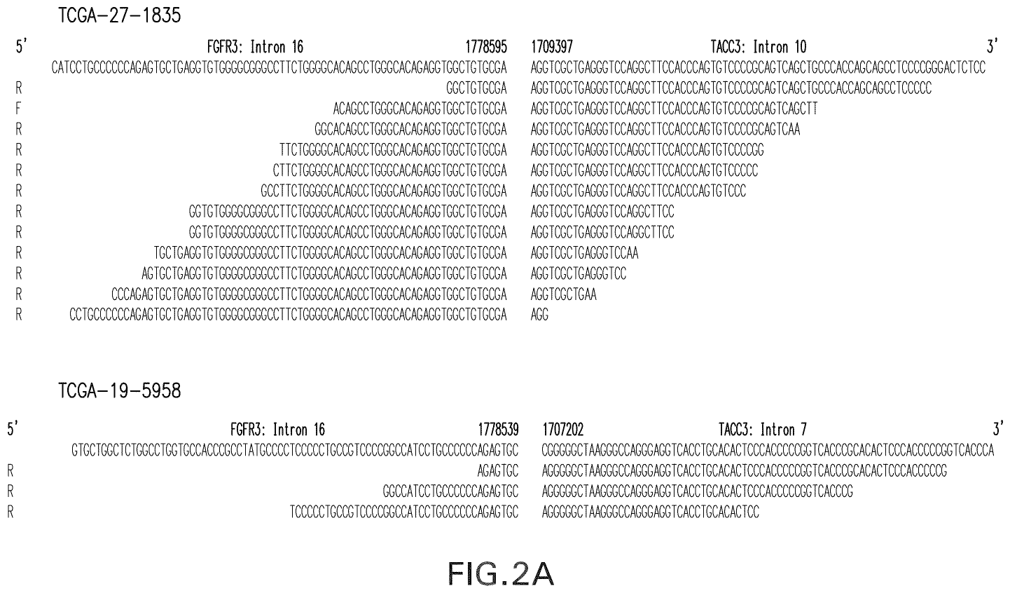

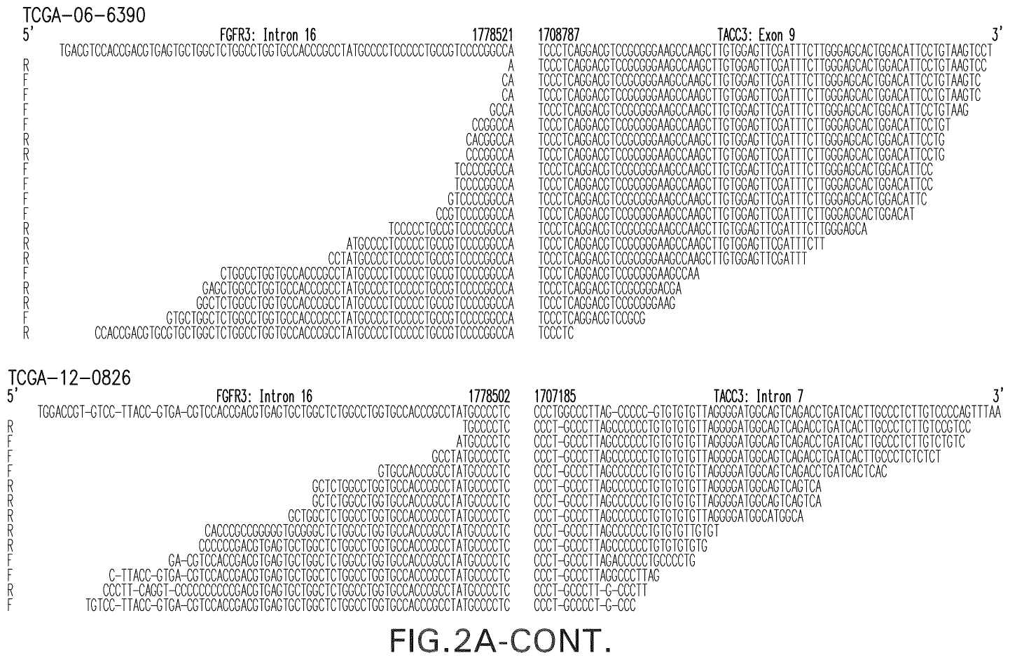

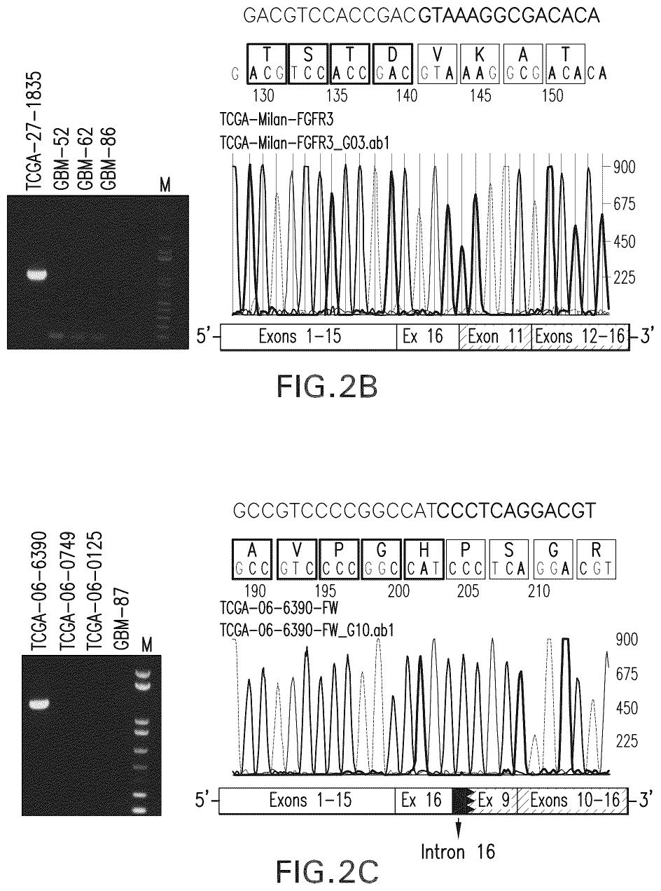

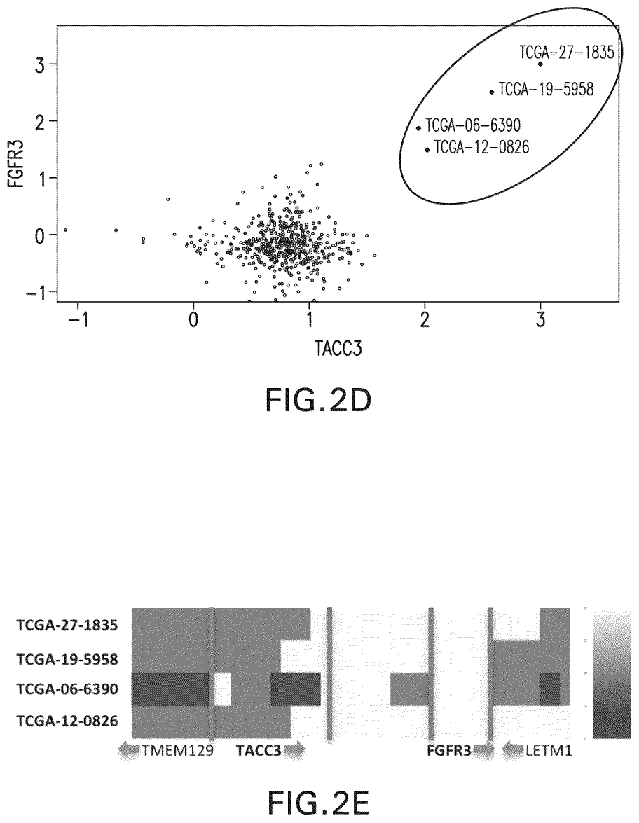

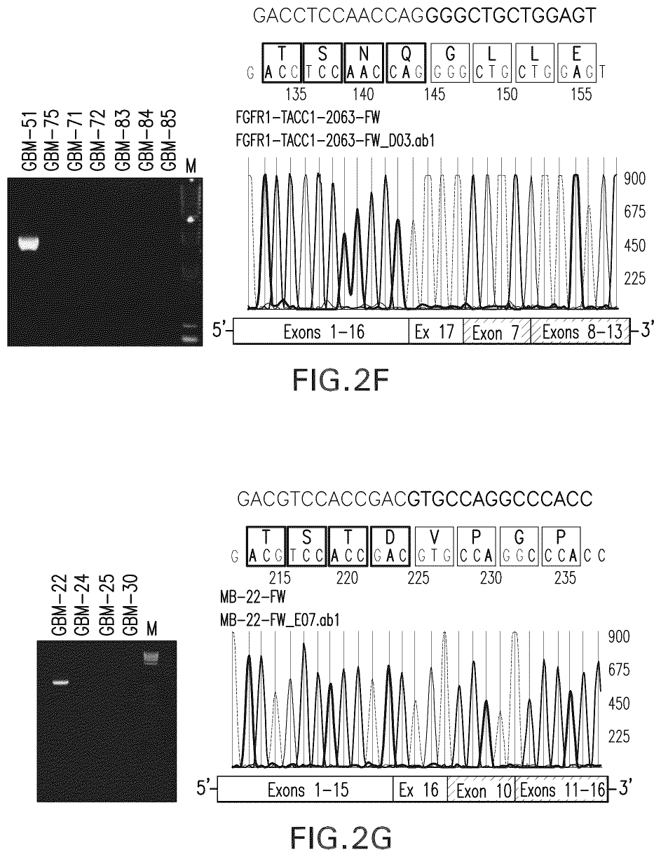

The experimental validation of the inferred genomic fusions was focused on FGFR3-TACC3. Exome-Fuse identified FGFR3-TACC3 gene fusions in four GBM samples with breakpoints spanning invariably within intron 16 of FGFR3 (which is downstream to the coding region for the TK domain) and intron 7-10 of TACC3 (which is upstream to the TACC domain) (FIG. 2A, Tables 4 and 5). Among the four positive TCGA GBM specimens, two were available from TCGA centers for molecular analysis (TCGA-27-1835 and TCGA-06-6390) and, by Sanger sequencing, each of them were confirmed to carry an in-frame fusion transcript that is consistent with the predicted genomic breakpoints (FIGS. 2B and 2C). Thus, the frames of the FGFR3-TACC3 fusion proteins invariably result in juxtaposing the TK domain of FGFR3 upstream of the TACC domain of TACC3. Consistent with the abundant expression of FGFR3-TACC3 in GSC-1123 and GBM-1123, the mRNA expression analysis of the TCGA tumors revealed that the four FGFR3-TACC3-positive GBM display marked co-outlier expression of FGFR3 and TACC3 (FIG. 2D). Recurrent gene fusions can be associated with local copy number variations (CNV) of the breakpoint regions (Wang et al., 2009). Accordingly, the analysis of SNP arrays in the TCGA dataset revealed the presence of microamplification events of the FGFR3 and TACC3 genes in all four FGFR3-TACC3-positive GBM (FIG. 2E).

TABLE 4
List of split inserts supporting the identification of FGFR3-TACC3 fusion genes in four GBM samples
from the ATLAS-TCGA exome collection (SEQ ID NOS 187-224, respectively, in order of appearance)

| | | gene1 | | % | | | | read | read | hg18 | hg18 | | bit | |
|---|---|---|---|---|---|---|---|---|---|---|---|---|---|---|
| TCGAsampleID | gene1 | length | readID | identity | length | mismatch | gap | start | end | genome | genome | e-value | score | read2 fasta |
| TCGA-06-6390 | FGFR3 | 76 | C01PRACXX110828:1:1301:1934:116558 | 100 | 76 | 0 | 0 | 1 | 76 | 1778372 | 1778447 | 8E-40 | 151 | GTGCTGGCATGCCGCGCC |
| | | | | | | | | | | | | | | CTCCCAGAGGCCCACCTT |
| | | | | | | | | | | | | | | CAAGCAGCTGGTGGAGGA |
| | | | | | | | | | | | | | | CCTGGACCGTGTCCTTAC |
| | | | | | | | | | | | | | | CGTG |
| TCGA-06-6390 | FGFR3 | 76 | C01RDACXX110828:3:2305:4872:47002 | 98.68 | 76 | 1 | 0 | 1 | 76 | 1778364 | 1778439 | 2E-37 | 143 | ATGCGGGAGTGCTGGCAT |
| | | | | | | | | | | | | | | GACGCGCCCTCCCAGAGG |
| | | | | | | | | | | | | | | CCCACCTTCAAGCAGCTG |
| | | | | | | | | | | | | | | GTGGAGGACCTGGACCGT |
| | | | | | | | | | | | | | | GTCC |
| TCGA-06-6390 | FGFR3 | 76 | D03U9ACXX110625:6:1203:16178:138219 | 100 | 76 | 0 | 0 | 1 | 76 | 1778413 | 1778488 | 8E-40 | 151 | AGCTGGTGGAGGACCTGG |
| | | | | | | | | | | | | | | ACCGTGTCCTTACCGTGA |
| | | | | | | | | | | | | | | CGTCCACCGACGTGAGTG |
| | | | | | | | | | | | | | | CTGGCTCTGGCCTGGTGC |
| | | | | | | | | | | | | | | CACC |
| TCGA-06-6390 | TACC3 | 76 | C01PRACXX110829:2:1102:13552:120312 | 100 | 76 | 0 | 0 | 1 | 76 | 1708918 | 1708843 | 8E-40 | 151 | CCCTTAAAACAACTCGTT |
| | | | | | | | | | | | | | | CCCTCAGACCACACACAA |
| | | | | | | | | | | | | | | GACAGTTCAAGAGGGACT |
| | | | | | | | | | | | | | | CAAGGACTTACAGGAATG |
| | | | | | | | | | | | | | | TCCA |
| TCGA-06-6390 | TACC3 | 76 | C01PRACXX11D628:8:2308:6515:60354 | 100 | 76 | 0 | 0 | 1 | 76 | 1709956 | 1708881 | 8E-40 | 151 | AACCAAAGGCTCAGACCC |
| | | | | | | | | | | | | | | CCAGGAATAGAAAATATA |
| | | | | | | | | | | | | | | GGCCCTTAAAACAACTCG |
| | | | | | | | | | | | | | | TTCCCTCAGACCACACAC |
| | | | | | | | | | | | | | | AAGA |
| TCGA-06-6390 | TACC3 | 76 | C01RDACXX110628:6:1305:16843:57213 | 98.68 | 76 | 1 | 0 | 1 | 76 | 1708865 | 1708780 | 2E-37 | 143 | TCAAGGACTTACAGGAAT |
| | | | | | | | | | | | | | | GTCCAGTGCTCCCAAGAA |
| | | | | | | | | | | | | | | ATCGAACTCCACAAGCTT |
| | | | | | | | | | | | | | | GGCTTCCCGCGCACGTCC |
| | | | | | | | | | | | | | | TGAG |
| TCGA-06-6390 | TACC3 | 75 | D03U9ACXX110625:2:1202:19578:90281 | 100 | 75 | 0 | 0 | 1 | 75 | 1708861 | 1708787 | 3E-39 | 149 | GGACTTACAGGAATGTCC |
| | | | | | | | | | | | | | | AGTGCTCCCAAGAAATCG |
| | | | | | | | | | | | | | | AACTCCACAAGCTTGGCT |
| | | | | | | | | | | | | | | TCCCGCGGACGTCCTGAG |
| | | | | | | | | | | | | | | GGAT |
| TCGA-06-6390 | TACC3 | 76 | D03U9ACXX110625:4:2306:2024:174970 | 100 | 76 | 0 | 0 | 1 | 76 | 1708869 | 1708821 | 8E-40 | 151 | CAGACCACACACAAGACA |
| | | | | | | | | | | | | | | GTTCAAGAGGGACTCAAG |
| | | | | | | | | | | | | | | GACTTACAGGAATGTCCA |
| | | | | | | | | | | | | | | GTGCTCCCAAGAAATCGA |
| | | | | | | | | | | | | | | ACTC |
| TCGA-12-0826 | FGFR3 | 72 | 61C59AAXX100217:4:21:17613:20556 | 98.61 | 72 | 0 | 1 | 1 | 71 | 1778439 | 1778510 | 2E-34 | 133 | CTTACCGTGACGTCCACC |
| | | | | | | | | | | | | | | GACGTGAGTGCTGGCTCT |
| | | | | | | | | | | | | | | GGCCTGGTGCCACCCGCC |
| | | | | | | | | | | | | | | TATGCCCCTCCCCTGCCC |
| | | | | | | | | | | | | | | TTAG |
| TCGA-12-0826 | TACC3 | 75 | 42MJNAAXX090813:5:30:1412:128060 | 100 | 75 | 0 | 0 | 2 | 76 | 1707299 | 1707225 | 3E-39 | 149 | AAACTTGAGGTATAAGGA |
| | | | | | | | | | | | | | | CTGCTTCCTCAAGGCCGA |
| | | | | | | | | | | | | | | CTCCTTAAACTGGGGACA |
| | | | | | | | | | | | | | | AGAGGGCAAGTGATCAGG |
| | | | | | | | | | | | | | | TCTG |
| TCGA-12-0826 | TACC3 | 76 | 61C59AAXX1002:17:4:2:4279:6948 | 100 | 76 | 0 | 0 | 1 | 76 | 1707209 | 1707224 | 8E-40 | 151 | AACTTGAGGTATAAGGAC |
| | | | | | | | | | | | | | | TGCTTGGTGAAGGCCGAC |
| | | | | | | | | | | | | | | TCCTTAAACTGGGGACAA |
| | | | | | | | | | | | | | | GAGGGCAAGTGATCAGGT |
| | | | | | | | | | | | | | | CTGA |
| TCGA-12-0826 | FGFR3 | 76 | 42MJNAAXX090813:5:37:435:1250#0 | 100 | 76 | 0 | 0 | 1 | 76 | 1778340 | 1778421 | 8E-40 | 151 | GCCCGCAGGTACATGATC |
| | | | | | | | | | | | | | | ATGCGGGAGTGCTGGCAT |
| | | | | | | | | | | | | | | GCCGCGCCCTCCCAGAGG |
| | | | | | | | | | | | | | | CCCACCTTCAAGCAGCTG |
| | | | | | | | | | | | | | | GTGG |
| TCGA-12-0826 | FGFR3 | 51 | 61C59AAXX100217:5:52:7727:2557 | 98.04 | 51 | 1 | 0 | 1 | 51 | 1778443 | 1778426 | 4E-24 | 93.7 | ACCGTGACGTCCACCGAC |
| | | | | | | | | | | | | | | GTGAGTGCTGGCTCTGGC |
| | | | | | | | | | | | | | | CTGGTGCGACCCGCCGAT |
| | | | | | | | | | | | | | | CTCTCTCCCCTGTCCTTT |
| | | | | | | | | | | | | | | TCCT |
| TCGA-19-5958 | TACC3 | 62 | D03U9ACXX110825:4:2208:9451:114169 | 90.32 | 62 | 6 | 0 | 1 | 62 | 1707141 | 1707202 | 4E-17 | 75.6 | TGGGAGGGTGCGGGGGGC |
| | | | | | | | | | | | | | | CGGGGGGGGGAGTGTGCA |
| | | | | | | | | | | | | | | GGTGAGCTCCCTGGCCCT |
| | | | | | | | | | | | | | | TGGCCCCCTGCCCTCTGG |
| | | | | | | | | | | | | | | GGGG |
| TCGA-19-5958 | TACC3 | 74 | D03U9ACXX110825:1:2204:20084:21192 | 98.95 | 74 | 3 | 0 | 1 | 74 | 1707097 | 1707170 | 5E-33 | 123 | CTGGGAATGGTGGTGTCT |
| | | | | | | | | | | | | | | CGGGCAGGGTTGTGGGTG |
| | | | | | | | | | | | | | | ACCGGGGGTGGGAGGGTG |
| | | | | | | | | | | | | | | CGGGGGACCGGGGGGGGG |
| | | | | | | | | | | | | | | AGGG |
| TCGA-27-1835 | FGFR3 | 76 | C00HWAEXX110325:7:2202:17660:110656 | 100 | 76 | 0 | 0 | 1 | 76 | 1778338 | 1778413 | 8E-40 | 151 | AGCGCCCTGCCCGCAGGT |
| | | | | | | | | | | | | | | ACATGATCATGCGGGAGT |
| | | | | | | | | | | | | | | GCTGGCATGCCGCGCCCT |
| | | | | | | | | | | | | | | CCCAGAGGCCCACCTTCA |
| | | | | | | | | | | | | | | AGCA |
| TCGA-27-1835 | TACC3 | 76 | C00HWAEXX110325:7:1104:10731:5183 | 100 | 76 | 0 | 0 | 1 | 76 | 1709417 | 1709417 | 8E-40 | 151 | GCCAACGCCATGCCCAGG |
| | | | | | | | | | | | | | | CCGGAGAGTCCCGGGGAG |
| | | | | | | | | | | | | | | GCTGCTGGTGGGCAGCTG |
| | | | | | | | | | | | | | | ACTGCGGGGACACTGGGT |
| | | | | | | | | | | | | | | GGAA |
| TCGA-27-1835 | TACC3 | 60 | B0972ABXX11D408:2:2201:5911:24541 | 100 | 60 | 0 | 0 | 1 | 60 | 1709445 | 1709445 | 3E-30 | 119 | AGGCCACCAGAGGCCAAC |
| | | | | | | | | | | | | | | GCCATGCCCAGGCCGGAG |
| | | | | | | | | | | | | | | AGTCCCGGGGAGGCTGCT |
| | | | | | | | | | | | | | | GGTGGGGAGGCGAACGCG |
| | | | | | | | | | | | | | | GGGA |
| TCGA-27-1835 | TACC3 | 61 | B097UABXX110405:4:2102:15742:63594 | 91.8 | 61 | 5 | 0 | 1 | 61 | 1709422 | 1709422 | 6E-19 | 91.9 | TGCCCAGGCCGGAGAGTC |
| | | | | | | | | | | | | | | CCGGGGCGGCTGCTGGGG |
| | | | | | | | | | | | | | | GGGAGCTGACTGGGGGGG |
| | | | | | | | | | | | | | | CACTGGGGGGGAGACCCG |
| | | | | | | | | | | | | | | GGCC |
| | | | | | | | | | | hg18 | hg18 | | | |
| | | gene2 | | % | | | | read | read | genome | genome | | bit | |
| TCGAsampleID | gene2 | length | readID | identity | length | mismatch | gap | start | end | start | end | e-value | score | read2 fasta |
| TCGA-06-6390 | TACC3 | 76 | C01FRACXX110628:1:1301:1934:116558 | 100 | 76 | 0 | 0 | 1 | 76 | 1708922 | 1708847 | 8E-40 | 151 | TAGGCCCTTAAAACAACT |
| | | | | | | | | | | | | | | CGTTCCCTCAGACCACAC |
| | | | | | | | | | | | | | | ACAAGACAGTTCAAGAGG |
| | | | | | | | | | | | | | | GACTCAAGGACTTACAGG |
| | | | | | | | | | | | | | | AATG |
| TCGA-06-6390 | TACC3 | 76 | C01RDACXX110629:3:2305:4872:47008 | 100 | 76 | 0 | 0 | 1 | 76 | 1708867 | 1708792 | 8E-40 | 151 | ACTCAAGGACTTACAGGA |
| | | | | | | | | | | | | | | ATGTCCAGTGCTCCCAAG |
| | | | | | | | | | | | | | | AAATCGAACTCCACAAGC |
| | | | | | | | | | | | | | | TTGGCTTCCCGCGGACGT |
| | | | | | | | | | | | | | | CCTG |
| TCGA-06-6390 | TACC3 | 76 | D03U9ACXX110625:6:1203:16178:138219 | 100 | 76 | 0 | 0 | 1 | 76 | 1708921 | 1708846 | 8E-40 | 151 | AGGCCCTTAAAACAACTC |
| | | | | | | | | | | | | | | GTTCCCTCAGACCACACA |
| | | | | | | | | | | | | | | CAAGACAGTTCAAGAGGG |
| | | | | | | | | | | | | | | ACTCAAGGACTTACAGGA |
| | | | | | | | | | | | | | | ATGT |
| TCGA-06-6390 | FGFR3 | 76 | C01PRACXX110828:2:1102:13552:120312 | 100 | 76 | 0 | 0 | 1 | 76 | 1778387 | 1778462 | 8E-40 | 151 | GCCCTCCCAGAGGCCCAC |
| | | | | | | | | | | | | | | CTTCAAGCAGCTGGTGGA |
| | | | | | | | | | | | | | | GGACCTGGACCGTGTCCT |
| | | | | | | | | | | | | | | TACCGTGACGTCCACCGA |
| | | | | | | | | | | | | | | CGTG |
| TCGA-06-6390 | FGFR3 | 76 | C01PRACXX110528:8:2308:6515:60354 | 100 | 76 | 0 | 0 | 1 | 76 | 1778382 | 1778457 | 8E-40 | 151 | GCCGCGCCCTCCCAGAGG |
| | | | | | | | | | | | | | | CCCACCTTCAAGCAGCTG |
| | | | | | | | | | | | | | | GTGGAGGACCTGGACCGT |
| | | | | | | | | | | | | | | GTCCTTACCGTGACGTCC |
| | | | | | | | | | | | | | | ACCG |
| TCGA-06-6390 | FGFR3 | 76 | C01RDACXX110628:6:1305:16843:57213 | 96.05 | 76 | 3 | 0 | 1 | 76 | 1778417 | 1778492 | 1E-32 | 127 | GGTGGAGGACCTGGACCG |
| | | | | | | | | | | | | | | TGACCTTACCGGGACGTC |
| | | | | | | | | | | | | | | CACCGACGGGAGTGCTGG |
| | | | | | | | | | | | | | | CTCTGGCCTGGTGCCACC |
| | | | | | | | | | | | | | | CGCC |
| TCGA-06-6390 | FGFR3 | 76 | D03U9ACXX110525:2:1202:19578:90281 | 100 | 76 | 0 | 0 | 1 | 76 | 1778447 | 1776522 | 8E-40 | 151 | GACGTCCACCGACGTGAG |
| | | | | | | | | | | | | | | TGCTGGCTCTGGCCTGGT |
| | | | | | | | | | | | | | | GCCACCCGCCTATGCCCC |
| | | | | | | | | | | | | | | TCCCCCTGCCGTCCCCGG |
| | | | | | | | | | | | | | | CCAT |
| TCGA-06-6390 | FGFR3 | 76 | D03U9ACXX110525:4:2306:2694:174970 | 100 | 76 | 0 | 0 | 1 | 76 | 1778435 | 1778510 | 8E-40 | 151 | TGTCCTTACCGTGACGTC |
| | | | | | | | | | | | | | | CACCGACGTGAGTGCTGG |
| | | | | | | | | | | | | | | CTCTGGCCTGGTGCCACC |
| | | | | | | | | | | | | | | CGCCTATGCCCCTCCCCC |
| | | | | | | | | | | | | | | TGCC |
| TCGA-12-0826 | TACC3 | 72 | 61C59AAXX100217:4:21:17613:20886 | 95.83 | 72 | 3 | 0 | 1 | 72 | 1707352 | 1707221 | 3E-30 | 119 | TACCTGCTGGTCTCGGTG |
| | | | | | | | | | | | | | | GCCACGGGCACTGGTCTA |
| | | | | | | | | | | | | | | CCAGGGCTGTCCCTCCGG |
| | | | | | | | | | | | | | | AGGGGGTCAAACTTGAGG |
| | | | | | | | | | | | | | | GATA |
| TCGA-12-0826 | FGFR3 | 76 | 42MJNAAXX090813:5:30:1412:1280#0 | 98.68 | 76 | 1 | 0 | 1 | 76 | 1778427 | 1778502 | 2E-37 | 143 | CTGGACCGTGTCCTTACC |
| | | | | | | | | | | | | | | GTGACGTCCACCGACGTG |
| | | | | | | | | | | | | | | AGTGCTGGCTCTGGCCTG |
| | | | | | | | | | | | | | | GTGCCACCCGCCCATGCC |
| | | | | | | | | | | | | | | CCTC |
| TCGA-12-0826 | FGFR3 | 76 | 61C59AAXX100217:4:2:4279:6949 | 98.68 | 75 | 0 | 1 | 1 | 75 | 1778435 | 1776510 | 8E-37 | 141 | TGTCCTTACCGTGACGTC |
| | | | | | | | | | | | | | | CACCGACGTGAGTGCTGG |
| | | | | | | | | | | | | | | CTCTGGCCTGGTGCCACC |
| | | | | | | | | | | | | | | CGCCTATGCCCCTCCCCT |
| | | | | | | | | | | | | | | GCCC |
| TCGA-12-0826 | TACC3 | 67 | 42MJNAAXX090813:5:37:435:1250#0 | 98.51 | 67 | 1 | 0 | 1 | 67 | 1707635 | 1707569 | 5E-32 | 125 | AAAAGATTTAAGTTTAGA |
| | | | | | | | | | | | | | | TCTTTAATATACCTAGAA |
| | | | | | | | | | | | | | | CGGTGGCTGTAACCAGCA |
| | | | | | | | | | | | | | | AGGCAGGAGCCCTTTGTG |
| | | | | | | | | | | | | | | TTGG |
| TCGA-12-0826 | TACC3 | 75 | 61C59AAXX100217:5:69:7727:2557 | 97.33 | 76 | 2 | 0 | 2 | 76 | 1707306 | 1707232 | 5E-36 | 133 | TGGGTCAAACTTGAGGTA |
| | | | | | | | | | | | | | | TAAGGACTGCTTCCTCAA |
| | | | | | | | | | | | | | | GGCCGACTCCTTATACTG |
| | | | | | | | | | | | | | | GGGACAAGAGGGCAAGTG |
| | | | | | | | | | | | | | | ATCA |
| TCGA-19-5958 | FGFR3 | 76 | D03U9ACXX110625:4:2206:9451:114168 | 98.68 | 76 | 1 | 0 | 1 | 76 | 1778462 | 1776537 | 2E-37 | 143 | GAGTGCTGGCTCTGGCCT |
| | | | | | | | | | | | | | | GGTGCCACCCGCCTATGC |
| | | | | | | | | | | | | | | CCCTCCCCCTGGCGTCCC |
| | | | | | | | | | | | | | | CGGCCATCCTGCCCCCCA |
| | | | | | | | | | | | | | | GAGT |
| TCGA-19-5958 | FGFR3 | 76 | D03U9ACXX110625:1:2204:20064:21192 | 100 | 76 | 0 | 0 | 1 | 76 | 1778462 | 1776537 | 2E-41 | 151 | GAGTGCTGGCTCTGGCCT |
| | | | | | | | | | | | | | | GGTGCCACCCGCCTATGC |
| | | | | | | | | | | | | | | CCCTCCCCCTGCCGTCCC |
| | | | | | | | | | | | | | | CGGCCATCCTGCCCCCCA |
| | | | | | | | | | | | | | | GAGT |
| TCGA-27-1835 | TACC3 | 76 | C00HWABXX110325:7:2202:17680:110566 | 96.05 | 76 | 3 | 0 | 1 | 76 | 1709492 | 1709417 | 1E-32 | 127 | GCCAACGCCATGCCCAGG |
| | | | | | | | | | | | | | | CCGGAGAGTCCCGGGGAG |
| | | | | | | | | | | | | | | GCTGCTGGTGGGGAGCTG |
| | | | | | | | | | | | | | | ACTTCGGGGACACTGGGG |
| | | | | | | | | | | | | | | GGAA |
| TCGA-27-1835 | FGFR3 | 76 | C00HWABXX110325:7:1104:10731:5183 | 96.05 | 76 | 3 | 0 | 1 | 76 | 1778363 | 1776438 | 1E-32 | 127 | CATGCGGGAGTGCTGGCA |
| | | | | | | | | | | | | | | TGGCGCGCCCTCCCAGCG |
| | | | | | | | | | | | | | | GCCCACCTTCAAGCAGCT |
| | | | | | | | | | | | | | | GGTGGGGGACCTGGACCG |
| | | | | | | | | | | | | | | TGTC |
| TCGA-27-1835 | FGFR3 | 76 | B09V2ABXX110408:2:2291:5811:24541 | 100 | 76 | 0 | 0 | 1 | 76 | 1778458 | 1778533 | 8E-40 | 151 | ACGTGAGTGCTGGCTCTG |
| | | | | | | | | | | | | | | GCCTGGTGCCACCCGCCT |
| | | | | | | | | | | | | | | ATGCCCCTCCCCCTGCCG |
| | | | | | | | | | | | | | | TCCCCGGCCATCCTGCCC |
| | | | | | | | | | | | | | | CCCA |
| TCGA-27-1835 | FGFR3 | 76 | B097UABXX110405:4:2102:15742:63594 | 100 | 76 | 0 | 0 | 1 | 76 | 1778388 | 1776453 | 8E-40 | 151 | CCCTCCCAGAGGCCCACC |
| | | | | | | | | | | | | | | TTCAAGCAGCTGGTGGAG |
| | | | | | | | | | | | | | | GACCTGGACCGTGTCCTT |
| | | | | | | | | | | | | | | ACCGTGACGTCCACCGAC |
| | | | | | | | | | | | | | | GTGA |

TABLE 5
List of split reads supporting the identification of FGFR3-TACC3 fusion genes in four GBM samples from the
ATLAS-TCGA exome collection (SEQ ID NOS 225-318, respectively, in order of appearance)

| sample | genesplit1 | readID | directionsplit | hg18startsplit1 | hg18stopsplit1 | length1 | mismatch1 | gap1 | seqsplit |
|---|---|---|---|---|---|---|---|---|---|
| TCGA-06-6390 | TACC3 | D03U9ACXX110025:2:1202:19578:90281 | R | 1778521 | 1778521 | 1 | 0 | 0 | GGACTTACAGGAATGTCCAG |
| | | | | | | | | | TGCTCCCAAGAAATCGAACT |
| | | | | | | | | | CCACAAGCTTGGCTTCCCGC |
| | | | | | | | | | GGACGTCCTGAGGGA***T |
| TCGA-06-6390 | FGFR3 | C01PRACXX11025:3:1104:10052:66371 | F | 1778520 | 1778521 | 2 | 0 | 0 | CA***TCCCTCAGGACGTCC |
| | | | | | | | | | GCGGGAAGCCAAGCTTGTGG |
| | | | | | | | | | AGTTCGATTTCTTGGGAGCA |
| | | | | | | | | | CTGGACATTCCTGTAAGTC |
| TCGA-06-6390 | FGFR3 | C01PRACXX11028:5:1108:3119:22892 | F | 1778520 | 1778521 | 2 | 0 | 0 | CA***TCCCTCAGGACGTCC |
| | | | | | | | | | GCGGGAAGCCAAGCTTGTGG |
| | | | | | | | | | AGTTCGATTTCTTGGGAGCA |
| | | | | | | | | | CTGGACATTCCTGTAAGTC |
| TCGA-06-6390 | FGFR3 | D03I9ACXX110025:8:2304:13007:108832 | F | 1778520 | 1778521 | 2 | 0 | 0 | CA***TCCCTCAGGACGTCC |
| | | | | | | | | | GCGGGAAGCCAAGCTTGTGG |
| | | | | | | | | | AGTTCGATTTCTTGGGAGCA |
| | | | | | | | | | CTGGACATTCCTGTAAGTC |
| TCGA-06-6390 | FGFR3 | C01PRACXX11028:5:2108:1999:91559 | F | 1778518 | 1778521 | 4 | 0 | 0 | GCCA***TCCCTCAGGACGT |
| | | | | | | | | | CCGCGGGAAGCCAAGCTTGT |
| | | | | | | | | | GGAGTTCGATTTCTTGGGAG |
| | | | | | | | | | CACTGGACATTCCTGTAAG |
| TCGA-06-6390 | FGFR3 | C01RDACXX110529:3:1336:1446:68211 | F | 1778515 | 1778521 | 7 | 0 | 0 | CCGGCCA***TCCCTGAGGA |
| | | | | | | | | | CGTCCGCGGGAAGCCAAGCT |
| | | | | | | | | | TGTGGAGTTCGATTTCTTGG |
| | | | | | | | | | GAGCACTGGACATTCCTGT |
| TCGA-06-6390 | TACC3 | D03U9ACXX110605:5:2205:12523:196352 | R | 1778514 | 1778521 | 8 | 1 | 0 | CAGGAATGTCCAGTGCTACC |
| | | | | | | | | | AAGAAATCGAACTCCACAAG |
| | | | | | | | | | CTTGGGTTCCCGCGGACGTC |
| | | | | | | | | | CTCCGGGA***TGGCCGTG |
| TCGA-06-6390 | TACC3 | C01PRACXX110628:5:2103:6315:17943 | R | 1778514 | 1778521 | 6 | 0 | 0 | CAGGAATGTCCAGTGCTCCC |
| | | | | | | | | | AAGAAATCGAACTCCACAAG |
| | | | | | | | | | CTTGGCTTCCCGCGGACGTC |
| | | | | | | | | | CTGAGGGA***TGGCCGGG |
| TCGA-06-6390 | FGFR3 | C01PRACXX110629:3:1204:10831:2928 | F | 1778512 | 1778521 | 10 | 0 | 0 | TCCCCGGCCA***TCCCTCA |
| | | | | | | | | | GGACGTCCGCGGGAAGCCAA |
| | | | | | | | | | GCTTGTGGAGTTCGATTTCT |
| | | | | | | | | | TGGGAGCACTGGACATTCC |
| TCGA-06-6390 | FGFR3 | C01PRACXX110828:5:2209:6732:191360 | F | 1778512 | 1778521 | 10 | 0 | 0 | TCCCCGGCCA***TCCCTCA |
| | | | | | | | | | GGAAGTCCGCGGGAAGCCAA |
| | | | | | | | | | GCTTGTGGAGTTCGATTTCT |
| | | | | | | | | | TGGGAGCACTGGACATTCC |
| TCGA-06-6390 | FGFR3 | C01PRACXX110628:8:1308:2911:20590 | F | 1778511 | 1778521 | 11 | 0 | 0 | GTCCCCGGCCA***TCCCTC |
| | | | | | | | | | AGGACGTCCGCGGGAAGCCA |
| | | | | | | | | | AGCTTGTGGAGTTCGATTTC |
| | | | | | | | | | TTGGGAGCACTGGACATTC |
| TCGA-06-6390 | FGFR3 | C01PRACXX110628:8:2207:4588:64017 | F | 1778509 | 1778521 | 13 | 0 | 0 | CCGTCCCCGGCCA***TCCC |
| | | | | | | | | | TCAGGACGTCCGCGGGAAGC |
| | | | | | | | | | CAAGCTTGTGGAGTTCGATT |
| | | | | | | | | | TCTTGGGAGCACTGGACAT |
| TCGA-06-6390 | TACC3 | C01PRACXX110628::2205:11925:39734 | R | 1778501 | 1778521 | 21 | 0 | 0 | TGCTCCCAAGAAATCGAACT |
| | | | | | | | | | CCACAAGCTTGGCTTCCCGC |
| | | | | | | | | | GGACGTCCTGAGGGA***TG |
| | | | | | | | | | GCCGGGGACGGCAGGGGGA |
| TCGA-06-6390 | TACC3 | C01PRACXX110528:6:1105:12159:179489 | R | 1778494 | 1778521 | 28 | 0 | 0 | AAGAAATCGAACTCCACAAG |
| | | | | | | | | | CTTGGCTTCCCGCGGACGTC |
| | | | | | | | | | CTGAGGGA***TGGCCGGGG |
| | | | | | | | | | ACGGCAGGGGGAGGGGCAT |
| TCGA-06-6390 | TACC3 | D03U9ACXX110625:4:2209:12501:40382 | R | 1778491 | 1778521 | 31 | 1 | 0 | AAATCGAACTCCACAAGCTT |
| | | | | | | | | | GGCTTCCCGCGGACGTCCTG |
| | | | | | | | | | AGGGA***TGGCCGGGGGCG |
| | | | | | | | | | GCAGGGGGAGGGGCATAGG |
| TCGA-06-6390 | FGFR3 | C01PRACXX110628:3:1305:3044:13239 | F | 1778473 | 1778521 | 49 | 0 | 0 | CTGGCCTGGTGCCACCCGCC |
| | | | | | | | | | TATGCCCCTCCCCCTGCCGT |
| | | | | | | | | | CCCCGGCCA***TCCCTCAG |
| | | | | | | | | | GACGTCCGCGGGAAGCCAA |
| TCGA-06-6390 | TACC3 | D03U9ACXX110625:5:2205:12523:195352 | R | 1778470 | 1778521 | 52 | 4 | 0 | TCGTCCCGCGGACTTCCTGA |
| | | | | | | | | | TGGA***TCGCCGGGGACGG |
| | | | | | | | | | CAGGGGGAGGGGCATAGGCG |
| | | | | | | | | | TGTGGCACCAGGCCAGCTC |
| TCGA-06-6390 | TACC3 | C01PRACXX110528:7:2205:11825:39734 | R | 1778469 | 1778521 | 53 | 1 | 0 | CTTCCCGCGGACGTCCTGAG |
| | | | | | | | | | GGA***TGGCCGGGGACGGA |
| | | | | | | | | | AGGGGGAGGGGCATAGGCGG |
| | | | | | | | | | GTGGCACCAGGCCAGAGCC |
| TCGA-06-6390 | FGFR3 | D03U9ACXX110525:7:2100:4492:193350 | F | 1778464 | 1778521 | 58 | 0 | 0 | GTGCTGGCTCTGGCCTGGTG |
| | | | | | | | | | CCACCCGCCTATGCCCCTCC |
| | | | | | | | | | CCCTGCCGTCCCCGGCCA** |
| | | | | | | | | | *TCCCTCAGGACGTCCGCG |
| TCGA-06-6390 | TACC3 | C01PRACXX110628:5:2103:6815:17943 | R | 1778452 | 1778521 | 70 | 0 | 0 | GAGGGA***TGGCCGGGGAC |
| | | | | | | | | | GGCAGGGGGAGGGGCATAGG |
| | | | | | | | | | CGGGTGGCACCAGGCCAGAG |
| | | | | | | | | | CCAGCACTCACGTCGGTGG |
| TCGA-12-0826 | TACC3 | B1C5RAAXX1D0217:4:93:15133:6133 | R | 1778495 | 1778502 | 6 | 0 | 0 | GGACAAGAGGGCAAGTGATC |
| | | | | | | | | | AGGTCTGACTGCCATCCCCT |
| | | | | | | | | | AACACACACAGGGGGGCTAA |
| | | | | | | | | | GGGCAGGG***GAGGGGCA |
| TCGA-12-0826 | TACC3 | B1C59AAXX100217:5:107:10875:15040 | R | 1778495 | 1778502 | 8 | 0 | 0 | GGACAAGAGGGCAAGTGATC |
| | | | | | | | | | AGGTCTGACTGCCATCCCCT |
| | | | | | | | | | AACACACACAGGGGGGCTAA |
| | | | | | | | | | GGGCAGGG***GAGGGGCA |
| TCGA-12-0826 | FGFR3 | B1C59AAXX100217:5:108:1909:11295 | F | 1778494 | 1778502 | 9 | 0 | 0 | ATGCCCCTC***CCCTGCCC |
| | | | | | | | | | TTAGCCCCCCTGTGTGTGTT |
| | | | | | | | | | AGGGGATGGCAGTCAGACCT |
| | | | | | | | | | GATCACTTGCCCTCTTGTC |
| TCGA-12-0826 | FGFR3 | B1C59AAXX100217:5:82:13129:10637 | F | 1778490 | 1778502 | 13 | 0 | 0 | GCCTATGCCCCTC***CCCT |
| | | | | | | | | | GCCCTTAGCCCCCCTGTGTG |
| | | | | | | | | | TGTTAGGGGATGGCAGTCAG |
| | | | | | | | | | ACCTGATCACTTGCCCTCT |
| TCGA-12-0826 | FGFR3 | 42MJNAAXX090813:5:80:891:1877#0 | F | 1778481 | 1778502 | 22 | 0 | 0 | GTGCCACCCGCCTATGCCCC |
| | | | | | | | | | TC***CCCTGCCCTTAGCCC |
| | | | | | | | | | CCCTGTGTGTGTTAGGGGAT |
| | | | | | | | | | GGCAGTCAGACCTGATCAC |
| TCGA-12-0826 | TACC3 | B1C59AAXX100217:3:75:30598:12001 | R | 1778470 | 1778502 | 33 | 1 | 0 | TGACTGCCATCCCCTAACAC |
| | | | | | | | | | ACACAGGGGGGCTAAGGGCA |
| | | | | | | | | | GGG***GAGGGGCATAGGCG |
| | | | | | | | | | GGGGGCACCAGGCCAGAGC |
| TCGA-12-0826 | TACC3 | B1C59AAXX100217:4:114:5844:3101 | R | 1778470 | 1778502 | 33 | 1 | 0 | TGACTGCCATCCCCTAACAC |
| | | | | | | | | | ACACAGGGGGGCTAAGGGCA |
| | | | | | | | | | GGG***GAGGGGCATAGGCG |
| | | | | | | | | | GGGGGCACCAGGCCAGAGC |
| TCGA-12-0826 | TACC3 | 42MJNAAXX0908136:70:652:108#0 | R | 1778458 | 1778502 | 37 | 3 | 0 | TGCCATCCCCTAACACACAC |
| | | | | | | | | | AGGGGGGCTAAGGGCAGGG* |
| | | | | | | | | | **GAGGGGCATAGGCGGGGG |
| | | | | | | | | | GCACCAGGACAGAGGCAGC |
| TCGA-12-0826 | TACC3 | B1C59AAXX100217:3:55:4955:15075 | R | 1778451 | 1778502 | 52 | 5 | 0 | CACACAGGGGGGCTAAGGGC |
| | | | | | | | | | AGGG***GAGGGGCATAGGC |
| | | | | | | | | | GGGGGGGACCAGGCCCGAGC |
| | | | | | | | | | CAGCACTCACGTCGGGGGG |
| TCGA-12-0826 | FGFR3 | 42MJNAAXX090813:5:23:158:1180#0 | F | 1778447 | 1778502 | 56 | 0 | 0 | GACGTCCACCGACGTGAGTG |
| | | | | | | | | | CTGGCTCTGGCCTGGTGCCA |
| | | | | | | | | | CCCGCCTATGCCCCTCC*** |
| | | | | | | | | | CCCTGCCCTTAGACCCCCTG |
| TCGA-12-0826 | FGFR3 | B1C59AAXX100217:4:21:17013:20886 | F | 1778439 | 1778502 | 64 | 0 | 0 | CTTACCGTGACGTCCACCGA |
| | | | | | | | | | CGTGAGTGCTGGCTCTGGCC |
| | | | | | | | | | TGGTGCCACCCGCCTATGCC |
| | | | | | | | | | CCTC***CCCTGCCCTTAG |
| TCGA-12-0826 | FGFR3 | B1C59AAXX100217:4:2:4279:5949 | F | 1778438 | 1778502 | 68 | 0 | 0 | TGTCCTTACCGTGACGTCCA |
| | | | | | | | | | CCGACGTGAGTGCTGGCTCT |
| | | | | | | | | | GGCCTGGTGCCACCCGCCTA |
| | | | | | | | | | TGCCCCTC***CCCTGCCC |
| TCGA-19-5958 | TACC3 | C01RDACXX110528:6:1102:11157:101952 | R | 1778533 | 1778539 | 7 | 0 | 0 | CGGGGGTGGGAGTGTGCGGG |
| | | | | | | | | | TGACCGGGGGTGGGAGTGTG |
| | | | | | | | | | CAGGTGACCTCCCTGGCCCT |
| | | | | | | | | | TAGCCCCCT***GCACTCT |
| TCGA-19-5958 | TACC3 | C01REACXX110629:2:2104:6009:99392 | R | 1778517 | 1778539 | 23 | 0 | 0 | CGGGTGACCGGGGGAGGGAG |
| | | | | | | | | | TGTGCAGGGGACCTCCCTGA |
| | | | | | | | | | GGGTTAGCCCCCT***GCAC |
| | | | | | | | | | TCTGGGGGGCAGGATGGCC |
| TCGA-19-5958 | TACC3 | C01PRACXX110022:7:2103:12434:91988 | R | 1778501 | 1778539 | 39 | 0 | 0 | GGAGTGTGCAGGTGACCTCC |
| | | | | | | | | | CTGGCCCTTAGCCCCCT*** |
| | | | | | | | | | GCACTCTGGGGGGCAGGATG |
| | | | | | | | | | GCCGGGGACGGCAGGGGGA |
| TCGA-27-1835 | TACC3 | B05UCABXX110322:5:1103:9252:48754 | R | 1778558 | 1778598 | 10 | 1 | 0 | GGGGAGGCTGCTGGTGGGCA |
| | | | | | | | | | GCTGACTGCGGGGACACTGG |
| | | | | | | | | | GAGGAAGCCTGGACCCTCAG |
| | | | | | | | | | CGAACT***TCGCCCAGCC |
| TCGA-27-1835 | FGFR3 | C00HWABXX110325:4:1201:20830:90877 | F | 1778557 | 1778598 | 20 | 0 | 0 | ACAGCCTGGGCACAGAGGTG |
| | | | | | | | | | GCTGTGCGA***AGGTCGCT |
| | | | | | | | | | GAGGGTCCAGGCTTCCACCC |
| | | | | | | | | | AGTGTCCCCGCAGTCAGCT |
| TCGA-27-1835 | TACC3 | B05UCABXX110322:5:1108:14043:83287 | R | 1778554 | 1778598 | 32 | 0 | 0 | TGACTGCGGGGACACTGGGT |
| | | | | | | | | | GGAAGCCTGGACCCTCAGCG |
| | | | | | | | | | ACCT***TCGCACAGCCACC |
| | | | | | | | | | TCTGTGCCCAGGCTGTGCC |
| TCGA-27-1835 | TACC3 | B097UABXX110405:4:2204:19443:99453 | R | 1778558 | 1778598 | 38 | 0 | 0 | CGGGGACACTGGGTGGAAGC |
| | | | | | | | | | CTGGACCCTCAGCGACCT** |
| | | | | | | | | | *TCGCACAGCCACCTCTGTG |
| | | | | | | | | | CCCAGGCTGTGCCCCAGAA |
| TCGA-27-1835 | TACC3 | B097UABXX110405:4:2201:20648:44401 | R | 1778557 | 1778598 | 39 | 2 | 0 | GGGGACACTGGGTGGAAGCC |
| | | | | | | | | | TGGACCCTCAGCGACCT*** |
| | | | | | | | | | TCGCACAGCCACCTCTGTGG |
| | | | | | | | | | CCAGGCTGTGCCACAGAAG |
| TCGA-27-1835 | TACC3 | B097UABXX110405:2:2104:15688:71022 | R | 1778555 | 1778598 | 41 | 0 | 0 | GGACACTGGGTGGAAGCCTG |
| | | | | | | | | | GACCCTCAGCGACCT***TC |
| | | | | | | | | | GCACAGCCACCTCTGTGCCC |
| | | | | | | | | | AGGCTGTGCCCCAGAAGGC |
| TCGA-27-1835 | TACC3 | C00HWABXX110325:5:2102:20394:42427 | R | 1778543 | 1778598 | 53 | 3 | 0 | GAAGCCTGGACCCTCAGCGA |
| | | | | | | | | | CCT***TCGCACAGCCACCT |
| | | | | | | | | | CTGTGCCCCGGCTGTGCCCC |
| | | | | | | | | | AGCCGGCCCGCCCCACACC |
| TCGA-27-1835 | TACC3 | B09V2ABXX110408:6:1203:19187:141962 | R | 1778543 | 1778598 | 53 | 0 | 0 | GAAGCCTGGACCCTCAGCGA |
| | | | | | | | | | CCT***TCGCACAGCCACCT |
| | | | | | | | | | CTGTGCCCAGGCTGTGCCCC |
| | | | | | | | | | AGAAGGCCCGCCCCACACC |
| TCGA-27-1835 | TACC3 | B09V2ABXX110408:8:1205:4774:91604 | R | 1778537 | 1778598 | 59 | 0 | 0 | TGGACCCTCAGCGACCT*** |
| | | | | | | | | | TCGCACAGCCACCTCTGTGC |
| | | | | | | | | | CCAGGCTGTGCCCCAGAAGG |
| | | | | | | | | | CCCGCCCCACACCTCAGCA |
| TCGA-27-1835 | TACC3 | C00HWABXX110325:2:1107:16165:23614 | R | 1778535 | 1778598 | 61 | 0 | 0 | GACCCTCAGCGACCT***TC |
| | | | | | | | | | GCACAGCCACCTCTGTGCCC |
| | | | | | | | | | AGGCTGTGCCCCAGAAGGCC |
| | | | | | | | | | CGCCCCACACCTCAGCACT |
| TCGA-27-1835 | TACC3 | C00HWABXX110325:7:2107:1225:187363 | R | 1778530 | 1778598 | 66 | 0 | 0 | TCAGCGACCT***TCGCACA |
| | | | | | | | | | GCCACCTCTGTGCCCAGGCT |
| | | | | | | | | | GTGCCCCAGAAGGCCCGCCC |
| | | | | | | | | | CACACCTCAGCACTCTGGG |
| TCGA-27-1835 | TACC3 | B097UABXX110405:2:214:15656:71022 | R | 1778523 | 1778598 | 73 | 0 | 0 | CCT***TCGCACAGCCACCT |
| | | | | | | | | | CTGTGCCCAGGCTGTGCCCC |
| | | | | | | | | | AGAAGGCCCGCCCCACACCT |
| | | | | | | | | | CAGCACTCTGGGGGGCAGG |
| sample | genesplit2 | readID | directionsplit | hg18startsplit2 | hg18stopsplit2 | length2 | mismatch2 | gap2 | seqmate |
| TCGA-06-6390 | FGFR3 | D03U9ACXX110025:2:1202:19578:90281 | R | 1708787 | 1708851 | 75 | 0 | 0 | GACGTCCACCGACGTGAGTG |
| | | | | | | | | | CTGGCTCTGGCCTGGTGCCA |
| | | | | | | | | | CCCGCCTATGCCCCTCCCCC |
| | | | | | | | | | TGCCGTCCCCGGCCAT |
| TCGA-06-6390 | TACC3 | C01PRACXX11025:3:1104:10052:66371 | F | 1708787 | 1708850 | 74 | 0 | 0 | CAAGAGGGACTCAAGGACTT |
| | | | | | | | | | ACAGGAATGTCCAGTGCTCC |
| | | | | | | | | | CAAGAAATCGAACTCCACAA |
| | | | | | | | | | GCTTGGCTTCCCGCGG |
| TCGA-06-6390 | TACC3 | C01PRACXX11028:5:1108:3119:22892 | F | 1708787 | 1708850 | 74 | 0 | 0 | CAAGAGGGACTCAAGGACTT |
| | | | | | | | | | ACAGGAATGTCCAGTGCTCC |
| | | | | | | | | | CAAGAAATCGAACTCCACAA |
| | | | | | | | | | GCTTGGCTTCCCGCGG |
| TCGA-06-6390 | TACC3 | D03I9ACXX110025:8:2304:13007:108832 | F | 1708787 | 1708850 | 74 | 0 | 0 | ATAGGCCCTTAAAACAACTC |
| | | | | | | | | | GTTCCCTCAGACCACACACA |
| | | | | | | | | | AGACAGTTCAAGAGGGACTC |
| | | | | | | | | | AAGGACTTACAGGAAT |
| TCGA-06-6390 | TACC3 | C01PRACXX11028:5:2108:1999:91559 | F | 1708787 | 1708858 | 72 | 0 | 0 | TCAAGAGGGACTCAAGGACT |
| | | | | | | | | | TACAGGAATGTCCAGTGCTC |
| | | | | | | | | | CCAAGAAATCGAACTCCACA |
| | | | | | | | | | AGCTTGGCTTCCCGCG |
| TCGA-06-6390 | TACC3 | C01RDACXX110529:3:1336:1446:68211 | F | 1708787 | 1705855 | 69 | 0 | 0 | ACCACACACAAGACAGTTCA |
| | | | | | | | | | AGAGGGACTCAAGGACTTAC |
| | | | | | | | | | AGGAATGTCCAGTGCTCCCA |
| | | | | | | | | | AGAAATCGAACTCCAC |
| TCGA-06-6390 | FGFR3 | D03U9ACXX110605:5:2205:12523:196352 | R | 1708787 | 1705854 | 68 | 4 | 0 | GAGCTGGCCTGGTGCCACAC |
| | | | | | | | | | GCCTATGCCCCTCCCCCTGC |
| | | | | | | | | | CGTCCCCGGCGATCCATCAG |
| | | | | | | | | | GAAGTCCGCGGGACGA |
| TCGA-06-6390 | FGFR3 | C01PRACXX110628:5:2103:6315:17943 | R | 1708787 | 1705854 | 68 | 0 | 0 | CCACCGACGTGAGTGCTGGC |
| | | | | | | | | | TCTGGCCTGGTGCCACCCGC |
| | | | | | | | | | CTATGCCCCTCCCCCTGCCG |
| | | | | | | | | | TCCCCGGCCATCCCTC |
| TCGA-06-6390 | TACC3 | C01PRACXX110629:3:1204:10831:2928 | F | 1708787 | 1703852 | 66 | 0 | 0 | CAAGAGCCTCAGACAGTGCA |
| | | | | | | | | | TGAGGGACCCGAGACAGTGC |
| | | | | | | | | | GGCGAGGGAACAGCACAGCG |
| | | | | | | | | | GCCCCATGCCCCCAAC |
| TCGA-06-6390 | TACC3 | C01PRACXX110828:5:2209:6732:191360 | F | 1708787 | 1703852 | 66 | 1 | 0 | CAAGAGCCTCAGACAGTGCA |
| | | | | | | | | | TGAGGGACCCGAGACAGTGC |
| | | | | | | | | | GGCGAGGGAACAGCACAGGG |
| | | | | | | | | | GCCCCATGCCCCCAAC |
| TCGA-06-6390 | TACC3 | C01PRACXX110628:8:1308:2911:20590 | F | 1708787 | 1708851 | 65 | 0 | 0 | CGTTCCCTCAGACCACACAC |
| | | | | | | | | | AAGACAGTTCAAGAGGGACT |
| | | | | | | | | | CAAGGACTTACAGGAATGTC |
| | | | | | | | | | CAGTGCTCCCAAGAGA |
| TCGA-06-6390 | TACC3 | C01PRACXX110628:8:2207:4588:64017 | F | 1708787 | 1708840 | 63 | 0 | 0 | CCAGGAATAGAAAATATAGG |
| | | | | | | | | | CCCTTAAAACAACTCGTTCC |
| | | | | | | | | | CTCAGACCACACACAAGACA |
| | | | | | | | | | GTTCAAGAGGGACTCA |
| TCGA-06-6390 | FGFR3 | C01PRACXX110628::2205:11925:39734 | R | 1708787 | 1708841 | 55 | 0 | 0 | GGCTCTGGCCTGGTGCCACC |
| | | | | | | | | | CGCCTATGCCCCTCCCCCTT |
| | | | | | | | | | CCGTCCCCGGCCATCCCTCA |
| | | | | | | | | | GGACGTCCGCGGGAAG |
| TCGA-06-6390 | FGFR3 | C01PRACXX110528:6:1105:12159:179489 | R | 1708787 | 1708834 | 48 | 0 | 0 | GCCCTGCCCGCAGGTACATG |
| | | | | | | | | | ATCATGCGGGAGTGCTGGCA |
| | | | | | | | | | TGCCGCGCCCTCCCAGAGGC |
| | | | | | | | | | CCACCTTCAAGCAGCT |
| TCGA-06-6390 | FGFR3 | D03U9ACXX110625:4:2209:12501:40382 | R | 1708787 | 1708831 | 45 | 0 | 0 | CTGGCATGCCGCGCCCTCCC |
| | | | | | | | | | AGAGGCCCACCTTTAAGCAG |
| | | | | | | | | | CTGGTAGAGGGCCTGGACCG |
| | | | | | | | | | TGTCCTTACCGTGACG |
| TCGA-06-6390 | TACC3 | C01PRACXX110628:3:1305:3044:13239 | F | 1708787 | 1708813 | 27 | 0 | 0 | TAAAACAACTCGTTCCCTCA |
| | | | | | | | | | GACCACACACAAGACAGTTC |
| | | | | | | | | | AAGAGGGACTCAAGGACTTA |
| | | | | | | | | | CAGGAATGTCCAGTGC |
| TCGA-06-6390 | FGFR3 | D03U9ACXX110625:5:2205:12523:195352 | R | 1708787 | 1708810 | 24 | 4 | 0 | CACGGCCATCCCGGAGGACG |
| | | | | | | | | | TCCGCGGGAACCCAAGCTTG |
| | | | | | | | | | TGGAGTTCGATTTCTTGGTA |
| | | | | | | | | | GCACTGGACATTCCTG |
| TCGA-06-6390 | FGFR3 | C01PRACXX110528:7:2205:11825:39734 | R | 1708787 | 1708809 | 23 | 0 | 0 | TCCCCCTGCCGTCCCCGGCC |
| | | | | | | | | | ATCCCTCAGGACGTCCGCGG |
| | | | | | | | | | GAAGCCAAGCTTGTGGAGTT |
| | | | | | | | | | CGATTTCTTGGGAGCA |
| TCGA-06-6390 | TACC3 | D03U9ACXX110525:7:2100:4492:193350 | F | 1708787 | 1705804 | 18 | 0 | 0 | AGACCACACACAAGACAGTT |
| | | | | | | | | | CAAGAGGGACTCAAGGACTT |
| | | | | | | | | | ACAGGAATGTCCAGTGCTCC |
| | | | | | | | | | CAAGAAATCGAACTCC |
| TCGA-06-6390 | FGFR3 | C01PRACXX110628:5:2103:6815:17943 | R | 1708787 | 1708702 | 6 | 0 | 0 | CCCGGCCATCCCTCAGGACG |
| | | | | | | | | | TCCGCGGGAAGCCAAGCTTG |
| | | | | | | | | | TGGAGTTCGATTTCTTGGGA |
| | | | | | | | | | GCACTGGACATTCCTG |
| TCGA-12-0826 | FGFR3 | B1C5RAAXX1D0217:4:93:15133:6133 | R | 1707185 | 1707253 | 69 | 2 | 1 | GGCATGCCGCGCCCTCCCAG |
| | | | | | | | | | AGGCCCACCTTCAAGCAGCT |
| | | | | | | | | | GGTGGAGGACCTGGACCGTG |
| | | | | | | | | | TCCTTACCGTGACGTC |
| TCGA-12-0826 | FGFR3 | B1C59AAXX100217:5:107:10875:15040 | R | 1707185 | 1707253 | 69 | 2 | 1 | GGCATGCCGCGCCCTCCCAG |
| | | | | | | | | | AGGCCCACCTTCAAGCAGCT |
| | | | | | | | | | GGTGGAGGACCTGGACCGTG |
| | | | | | | | | | TCCTTACCGTGACGTC |
| TCGA-12-0826 | TACC3 | B1C59AAXX100217:5:108:1909:11295 | F | 1707185 | 1707252 | 68 | 2 | 1 | CGGCGCACATACCTGCTGGT |
| | | | | | | | | | CTCGGTGGCCACGGGCACTG |
| | | | | | | | | | GTCTACCAGGACTGTCCCTC |
| | | | | | | | | | AGGAGGGGGTCAAACT |
| TCGA-12-0826 | TACC3 | B1C59AAXX100217:5:82:13129:10637 | F | 1707185 | 1707248 | 64 | 2 | 1 | ATACCTGCTGGTCTCGGTGG |
| | | | | | | | | | CCACGGGCACTGGTCTACCA |
| | | | | | | | | | GGACTGTCCCTCAGGAGGGG |
| | | | | | | | | | GTCAAACTTGAGGTAT |
| TCGA-12-0826 | TACC3 | 42MJNAAXX090813:5:80:891:1877#0 | F | 1707185 | 1707239 | 55 | 2 | 1 | AGGTATAAGGACTGCTTCCT |
| | | | | | | | | | CAAGGCCGACTCCTTAAACT |
| | | | | | | | | | GGGGACAAGAGGGCAAGTGA |
| | | | | | | | | | TCAGGTCTGACTGCCA |
| TCGA-12-0826 | FGFR3 | B1C59AAXX100217:3:75:30598:12001 | R | 1707185 | 1707228 | 44 | 2 | 1 | GGAGGACCTGGACTGTGTCC |
| | | | | | | | | | TTACCGTGACGTCCACCGAC |
| | | | | | | | | | GTGAGTGCTGGCTCTGGCCT |
| | | | | | | | | | GGTGCCACCCGCCTAT |
| TCGA-12-0826 | FGFR3 | B1C59AAXX100217:4:114:5844:3101 | R | 1707185 | 1707228 | 44 | 2 | 1 | GGAGGACCTGGACCGTGTCC |
| | | | | | | | | | TTACCGTGACGTCCACCGAC |
| | | | | | | | | | GTGAGTGCTGGCTCTGGCCT |
| | | | | | | | | | GGTGCCACCCGCCTAT |
| TCGA-12-0826 | FGFR3 | 42MJNAAXX0908136:70:652:108#0 | R | 1707185 | 1707224 | 40 | 2 | 1 | CAAGCAGCTGGTGGAGGACC |
| | | | | | | | | | TGGACCGTGTCCTTACCGTG |
| | | | | | | | | | ACGTCCACCGACGTGAGTGC |
| | | | | | | | | | TGGCTCTGGCCTGGTG |
| TCGA-12-0826 | FGFR3 | B1C59AAXX100217:3:55:4955:15075 | R | 1707185 | 1707209 | 25 | 2 | 1 | ACCTTCAAGCAGCTGGTGGA |
| | | | | | | | | | GGACCTGGACCGTGTCCTTA |
| | | | | | | | | | CCGTGACGTCCACCGACGTG |
| | | | | | | | | | AGTGCTGGCTCTGGCC |
| TCGA-12-0826 | TACC3 | 42MJNAAXX090813:5:23:158:1180#0 | F | 1707185 | 1707205 | 21 | 2 | 1 | CAAACTTGAGGTATAAGGAC |
| | | | | | | | | | TGCTTCCTCAAGGCCGACTC |
| | | | | | | | | | CTTAAACTGGGGACAAGAGG |
| | | | | | | | | | GCAAGTGATCAGGTCT |
| TCGA-12-0826 | TACC3 | B1C59AAXX100217:4:21:17013:20886 | F | 1707185 | 1707197 | 13 | 0 | 1 | TACCTGCTGGTCTCGGTGGC |
| | | | | | | | | | CACGGGCACTGGTCTACCAG |
| | | | | | | | | | GGCTGTCCCTCCGGAGGGGG |
| | | | | | | | | | TCAAACTTGAGGGATA |
| TCGA-12-0826 | TACC3 | B1C59AAXX100217:4:2:4279:5949 | F | 1707185 | 1707193 | 8 | 0 | 1 | AACTTGAGGTATAAGGACTG |
| | | | | | | | | | CTTCCTCAAGGCCGACTCCT |
| | | | | | | | | | TAAACTGGGGACAAGAGGGC |
| | | | | | | | | | AAGTGATCAGGTCTGA |
| TCGA-19-5958 | FGFR3 | C01RDACXX110528:6:1102:11157:101952 | R | 1707202 | 1707270 | 69 | 1 | 0 | AGCTGGTGGAGGACCTGGAC |
| | | | | | | | | | CGTGTCCTTACCGTGACGTC |
| | | | | | | | | | CACCGACGTGAGTGCTGGCT |
| | | | | | | | | | CTGGCCTGGTGCCACC |
| TCGA-19-5958 | FGFR3 | C01REACXX110629:2:2104:6009:99392 | R | 1707202 | 1707254 | 53 | 3 | 0 | GCGCCCTCCCAGAGGCCCAC |
| | | | | | | | | | CTTCAAGCAGCTGGTGGAGG |
| | | | | | | | | | ACCTGGACCGTGTCCTTACC |
| | | | | | | | | | GTGACGTCCACCGACG |
| TCGA-19-5958 | FGFR3 | C01PRACXX110022:7:2103:12434:91988 | R | 1707202 | 1707238 | 37 | 1 | 0 | GCGGGAGTGCTGGCATGCCG |
| | | | | | | | | | CGCCCTCCCAGAGGCCCACC |
| | | | | | | | | | TTCAAGCAGCTGGTGGAGGA |
| | | | | | | | | | CCTGGACCGTGTCCTT |
| TCGA-27-1835 | FGFR3 | B05UCABXX110322:5:1103:9252:48754 | R | 1709397 | 1709452 | 66 | 2 | 0 | CCTCCACTGGGTCCTCAGGG |
| | | | | | | | | | GTGGGGGTCCCTCCGGGGCT |
| | | | | | | | | | GGGCGGGGGAGGGACTGGCA |
| | | | | | | | | | GGCCTGCAGGGGGGTT |
| TCGA-27-1835 | TACC3 | C00HWABXX110325:4:1201:20830:90877 | F | 1709397 | 1709443 | 47 | 0 | 0 | TCACGGCAGCAAGAACCACA |
| | | | | | | | | | CTCACTGCTGCAAGGCCACC |
| | | | | | | | | | AGAGGCCAACGCCATGCCCA |
| | | | | | | | | | GGCCGGAGAGTCCCGG |
| TCGA-27-1835 | FGFR3 | B05UCABXX110322:5:1108:14043:83287 | R | 1709397 | 1709440 | 44 | 0 | 0 | TACATGATCATGCGGGAGGG |
| | | | | | | | | | CTGGCATGCCGCGCCCTCCC |
| | | | | | | | | | AGAGGCCCACCTTCAAGCAG |
| | | | | | | | | | CTGGTGGAGGGCCGGG |
| TCGA-27-1835 | FGFR3 | B097UABXX110405:4:2204:19443:99453 | R | 1709397 | 1709434 | 38 | 0 | 0 | GGTGGGAAGCGGCGGGGCTC |
| | | | | | | | | | ACTCCTGAGCGCCCTGCCCG |
| | | | | | | | | | CAGGGACATGATCATGCGGG |
| | | | | | | | | | GGTGCTGGCCTTGCGG |
| TCGA-27-1835 | FGFR3 | B097UABXX110405:4:2201:20648:44401 | R | 1709397 | 1709433 | 37 | 0 | 0 | GCGCCCTCCCAGAGGCCCAC |
| | | | | | | | | | CTTCAAGCAGCTGGTGGAGG |
| | | | | | | | | | ACCTGGACCGTGTCCTTACC |
| | | | | | | | | | GTGACGTCCACCGACG |
| TCGA-27-1835 | FGFR3 | B097UABXX110405:2:2104:15688:71022 | R | 1709397 | 1709431 | 35 | 0 | 0 | CCTGCCCCCCAGAGTGCTGA |
| | | | | | | | | | GGTGTGGGGCGGGCCTTCTG |
| | | | | | | | | | GGGCACAGCCTGGGCACAGA |
| | | | | | | | | | GGTGGCTGTGCGAAGG |
| TCGA-27-1835 | FGFR3 | C00HWABXX110325:5:2102:20394:42427 | R | 1709397 | 1709419 | 23 | 0 | 0 | GCAGGTACATGATCATGCGG |
| | | | | | | | | | GAGTGCCGGCATTTCGGGAC |
| | | | | | | | | | CTTCCCTCGGGCCACCCTCT |
| | | | | | | | | | TCCGGTTGTTGTGGGC |
| TCGA-27-1835 | FGFR3 | B09V2ABXX110408:6:1203:19187:141962 | R | 1709397 | 1709419 | 23 | 0 | 0 | GCAGGTACATGATCATGCGG |
| | | | | | | | | | GAGTGCTGGCATGCCGCGCC |
| | | | | | | | | | CTCCCCGAGGACCACCTTCC |
| | | | | | | | | | AGCAGCCGGGGGAGGG |
| TCGA-27-1835 | FGFR3 | B09V2ABXX110408:8:1205:4774:91604 | R | 1709397 | 1709413 | 17 | 0 | 0 | CCCGAATAAGGTGGGAAGCG |
| | | | | | | | | | GCGGGGCTCACTCCTGAGCG |
| | | | | | | | | | CCCTGACCGCAGGTACATGA |
| | | | | | | | | | GCATGCGGGAGTGGCG |
| TCGA-27-1835 | FGFR3 | C00HWABXX110325:2:1107:16165:23614 | R | 1709397 | 1709411 | 16 | 0 | 0 | CGTGTCCTTACCGTGACGTC |
| | | | | | | | | | CACCGACGTGAGTGCTGGCT |
| | | | | | | | | | CTGGCCTGGTGCCACCCGCC |
| | | | | | | | | | TATGCCCCTCCCCCTG |
| TCGA-27-1835 | FGFR3 | C00HWABXX110325:7:2107:1225:187363 | R | 1709397 | 1709400 | 10 | 0 | 0 | ACATGATCATGCGGGAGTGC |
| | | | | | | | | | TGGCATGCCGCGCCCCCCCA |
| | | | | | | | | | GAGGCCCACCTTCAAGCAGC |
| | | | | | | | | | TGGTGGAGGACCTGGA |
| TCGA-27-1835 | FGFR3 | B097UABXX110405:2:214:15656:71022 | R | 1709397 | 1709390 | 3 | 0 | 0 | GCCTTCTGGGGCACAGCCTG |
| | | | | | | | | | GGCACAGAGGTGGCTGTGCG |
| | | | | | | | | | AAGGTCGCTGAGGGTCCAGG |
| | | | | | | | | | CTTCCACCCAGTGTCC |

The FGFR3 and TACC3 genes are located 48-Kb apart on human chromosome 4p16. The other members of the FGFR and TACC families retain the close physical association of FGFR3 and TACC3, with FGFR1 and TACC1 paired on chromosome 8p11 and FGFR2 and TACC2 paired on chromosome 10q26. Without being bound by theory, the ancestral FGFR and TACC genes were physically linked and that this tandem gene cluster was duplicated at least twice to generate the FGFR1-TACC1, FGFR2-TACC2 and FGFR3-TACC3 pairs that mark mammalian evolution (Still et al., 1999). The highly conserved TK domains among FGFR genes and TACC domains among TACC genes together with their invariable fusion in the FGFR3-TACC3 rearrangements prompted to ask whether other intra-chromosomal FGFR-TACC fusion combinations exist in human GBM.

cDNA from a panel of 88 primary GBM were screened using pairs of upstream PCR primers that bind the amino-terminal coding region of the TK domains of FGFR1, FGFR2 and FGFR3 and downstream primers that bind to the carboxy-terminal coding region of the TACC domains of TACC1, TACC2 and TACC3 genes, respectively. The screening resulted in the identification of intrachromosomal FGFR-TACC fusions in two additional cases (one harboring FGFR1-TACC1 and one FGFR3-TACC3), corresponding to three of 97 total GBM (3.1%), including the GBM-1123 case. The FGFR1-TACC1 fusion breakpoint in GBM-51 joined in-frame exon 17 of FGFR1 to exon 7 of TACC1, resulting in a novel protein in which the TK domain of FGFR1 is fused upstream of the TACC domain of TACC1 (FIG. 2F). The same structure was conserved again in GBM-22 in which exon 16 of FGFR3 is joined in-frame to exon 10 of TACC3 (FIG. 2G). None of the tumors harboring FGFR-TACC fusions had mutations in IDH1 or IDH2 genes, thus indicating that FGFR-TACC-positive GBM mark an independent subgroup of patients from those carrying IDH mutations (Table 6) (Yan et al., 2009). The constant linkage of the FGFR-TK to the TACC domain created in each of the seven GBM harboring FGFR-TACC rearrangements suggests that FGFR-TACC fusion proteins may generate important functional consequences for oncogenesis in the brain.

TABLE 6

| | | | | Age at initial | IDH1-2 status | IDH1-2 status |
|---|---|---|---|---|---|---|
| Samples | Type | Time | Status | pathologic diagnosis | (Sanger) | (exome) |
| TCGA-12-0826 | FGFR3-TACC3 | 845 | DECEASED | 38 | WT | WT |
| TCGA-27-1635 | FGFR3-TACC3 | 646 | DECEASED | 53 | NA | WT |
| TCGA-19-5958 | FGFR3-TACC3 | 164 | LIVING | 56 | NA | WT |
| TCGA-06-6390 | FGFR3-TACC3 | 163 | DECEASED | 58 | WT | WT |
| GBM-22 | FGFR3-TACC3 | 390 | DECEASED | 60 | WT | NA |
| GBM-1123 | FGFR3-TACC3 | NA | DECEASED | 62 | WT | NA |
| GBM-51 | FGFR1-TACC1 | NA | NA | NA | WT | NA |

Time = Survival (days after diagnosis)
Sanger = analysis done by Sanger sequencing of genomic DNA
Exome = ainalysis done by the SAVI (Statistical Algorithm for Variant Identification), an algorithm developed to detect point mutation in cancer (BRAF Mutations in Hairy-Cell Leukemia, Tiacci E et al. The New England Journal of Medicine 2011 Jun 16; 364(24): 2305-15)
NA = Not Available
WT = Wild type sequence for R132 and R172 of IDH1 and IDH2, respectively

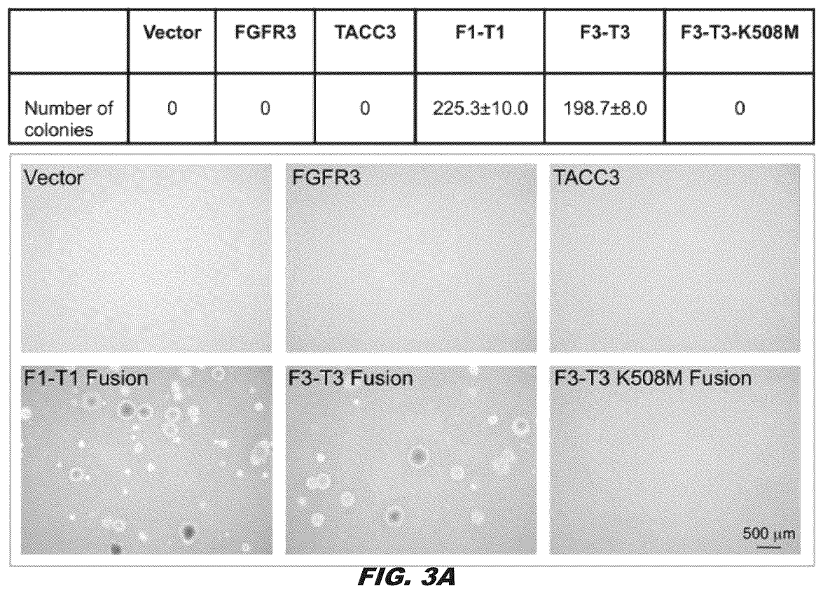

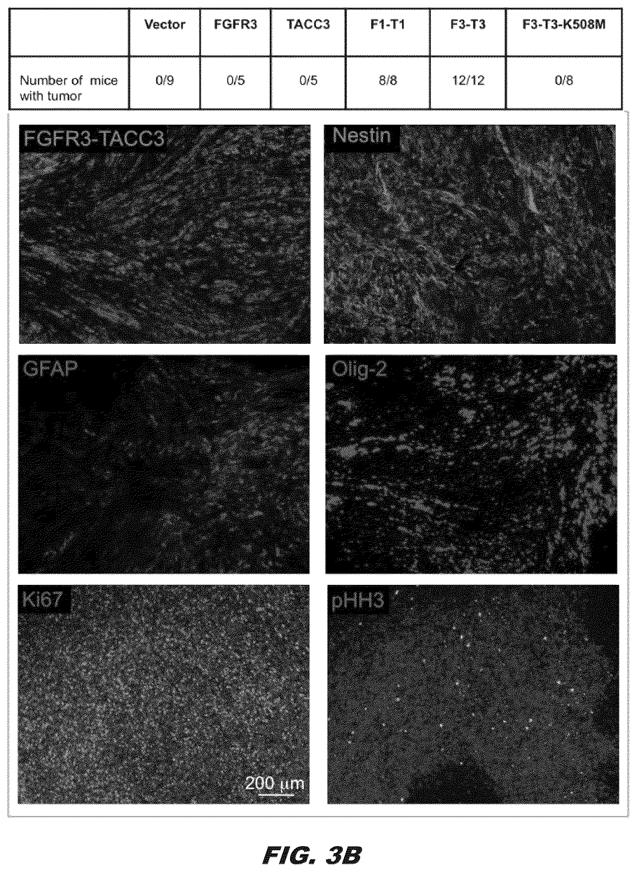

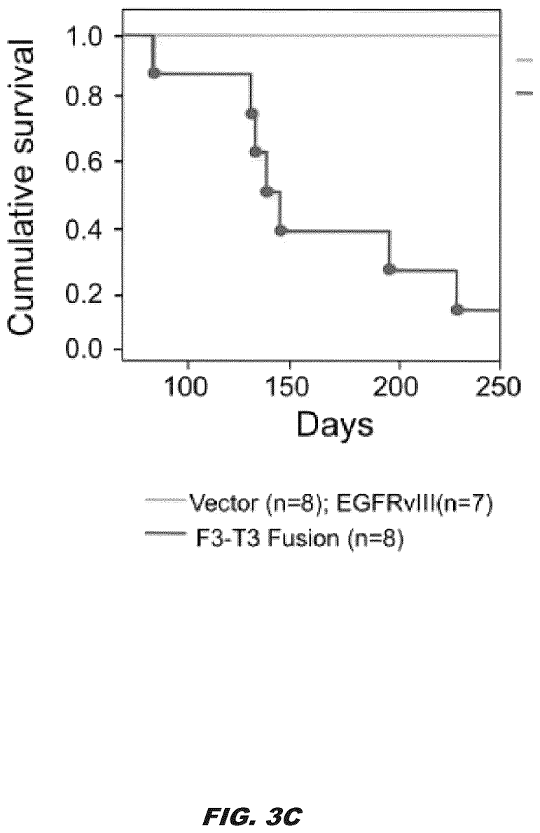

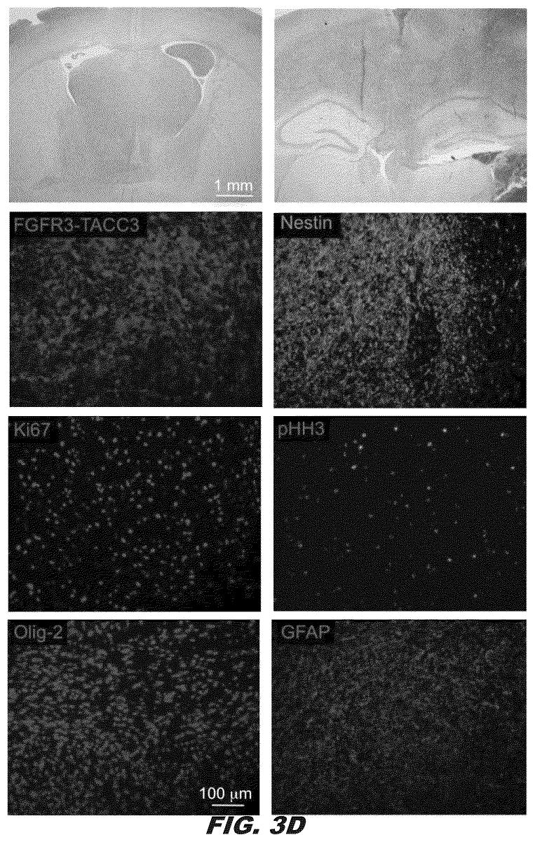

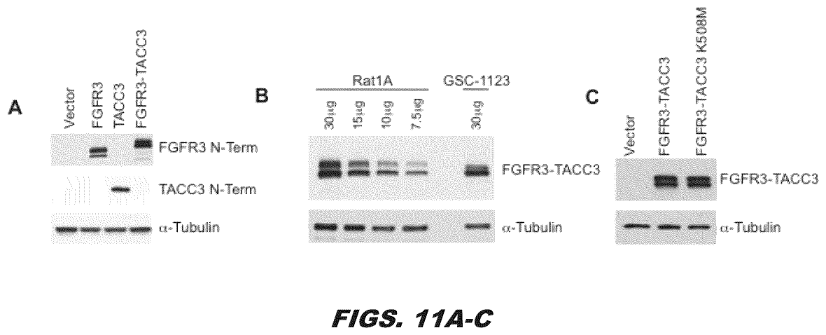

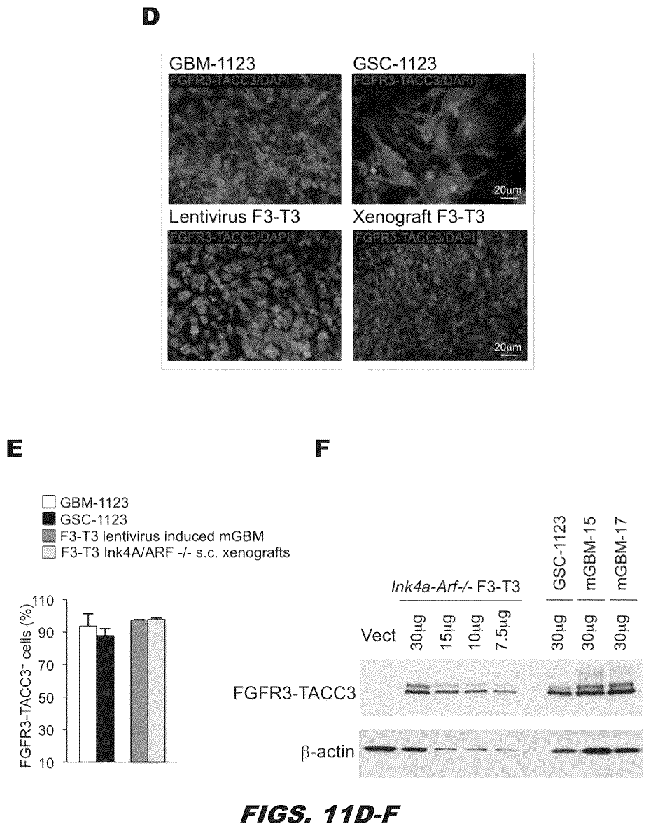

Transforming activity of FGFR-TACC fusions. To test the functional importance of the FGFR-TACC fusions in GBM, the FGFR3-TACC3 cDNA was cloned from GSC-1123 and recombinant lentiviruses were prepared expressing FGFR3-TACC3, FGFR1-TACC1, a kinase-dead FGFR3-TACC3 protein (FGFR3-TACC3-K508M), wild type FGFR3 and wild type TACC3. Transduction of Rat1A fibroblasts and Ink4A;Arf−/− astrocytes with the FGFR3-TACC3 lentivirus resulted in the expression of the fusion protein at levels comparable to those present in GSC-1123 (FIG. 11). Having reconstituted in non-transformed cells the endogenous level of the FGFR-TACC protein that accumulates in GBM cells, it was determined whether it was sufficient to initiate oncogenic transformation in vitro and in vivo. Rat1A cells expressing FGFR3-TACC3 and FGFR1-TACC1 but not those expressing FGFR3-TACC3-K508M, FGFR3, TACC3 or the empty lentivirus acquired the ability to grow in anchorage-independent conditions in soft agar (FIG. 3A). Transduction of the same lentiviruses in primary Ink4A;Arf−/− astrocytes followed by subcutaneous injection into immunodeficient mice revealed that only astrocytes expressing FGFR3-TACC3 and FGFR1-TACC1 formed tumors. The tumors emerged in 100% of the mice injected with astrocytes expressing the fusion proteins and were glioma-like lesions with strong positivity for Ki67, phospho-histone H3, nestin, GFAP and Olig2 (FIG. 3B).

Next, it was determined whether the FGFR3-TACC3 fusion protein is oncogenic when transduced to a small number of cells directly into the brain of immunocompetent animals. A recently described mouse glioma model was used in which brain tumors are initiated by lentiviral transduction of oncogenes and inactivation of p53 in the mouse brain (Marumoto et al., 2009). To target adult NSCs, the adult mouse hippocampus was stereotactically transduced with purified lentivirus expressing the FGFR3-TACC3 protein and shRNA against p53 (pTomo-FGFR3-TACC3-shp53). Seven of eight mice (87.5%) transduced with FGFR3-TACC3 succumbed from malignant brain tumors within 240 days (FIG. 3C). None of the mice transduced with a lentivirus expressing the most frequent gain-of-function mutation in GBM (the constitutively active EGFRvII, pTomo-EGFRvIII-shp53) or the pTomo-shp53 control lentivirus died or developed clinical signs of brain tumors (FIG. 3C). The FGFR3-TACC3 tumors were high-grade glioma with strong propensity to invade the normal brain and stained positive for the glioma stem cell markers nestin and Olig2 and the glial marker GFAP. They were also highly positive for Ki67 and phospho-histone H3, thus displaying rapid tumor growth (FIG. 3D). The expression of FGFR3-TACC3 in the xenograft and intracranial tumor models was comparable to the expression of the endogenous protein in the human GSCs and tumor (FIGS. 11D, 11E and 11F).

These data show that FGFR-TACC fusion proteins possess transforming activity in two independent cellular models and this activity is not the result of the overexpression of individual FGFR and TACC genes. They also show that direct transduction of the FGFR3-TACC3 protein to the adult mouse brain leads to efficient development of malignant glioma.

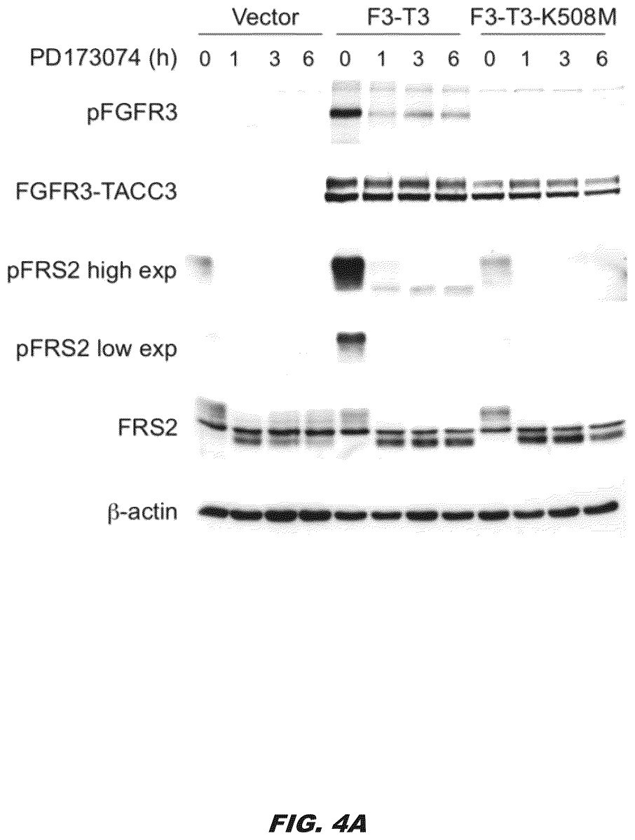

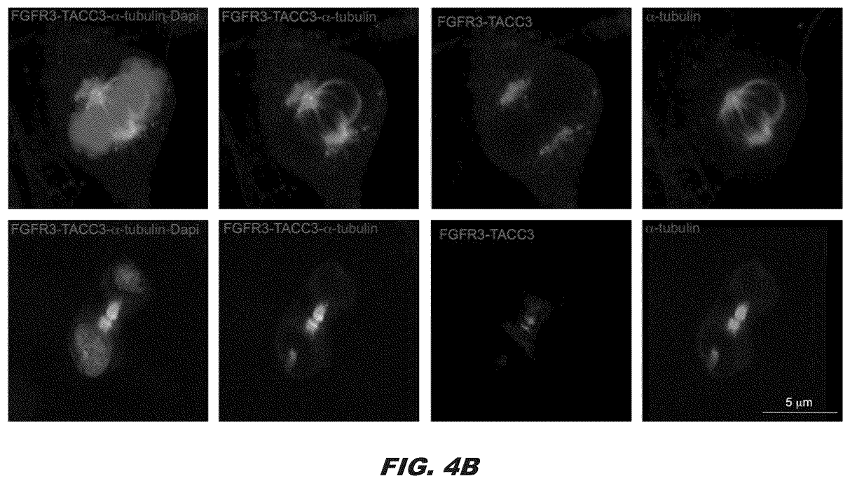

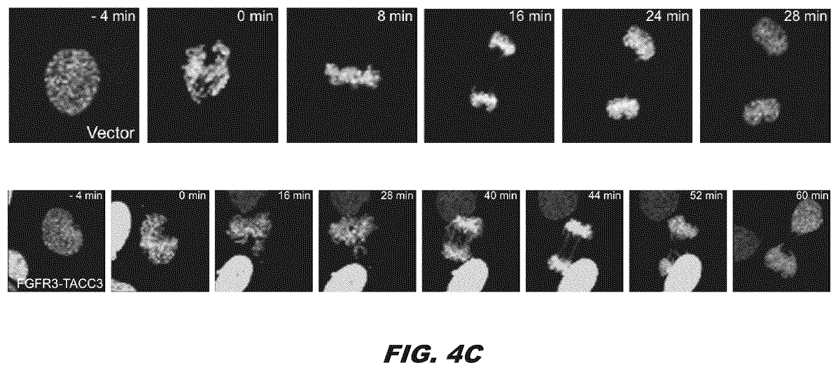

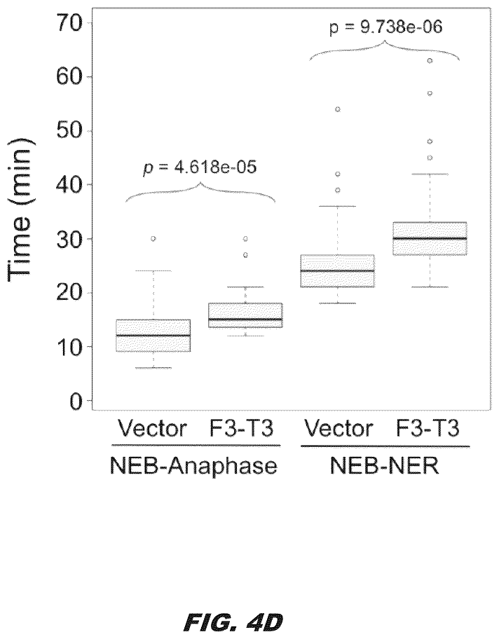

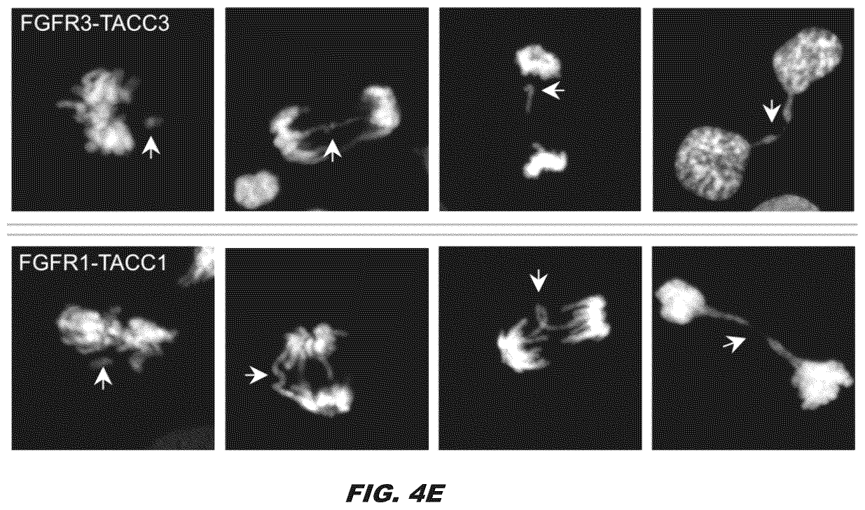

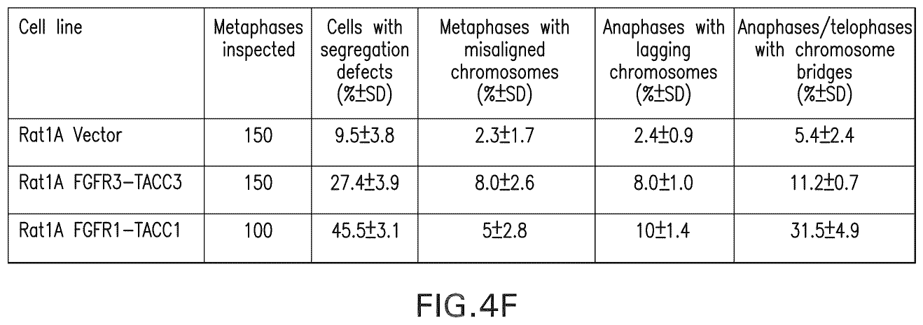

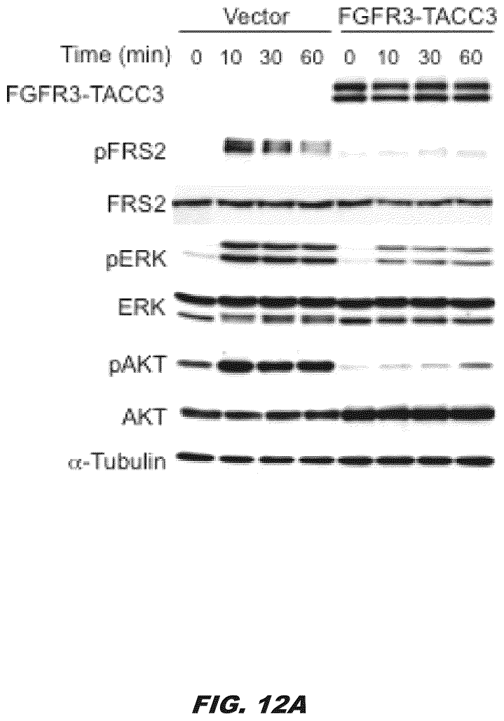

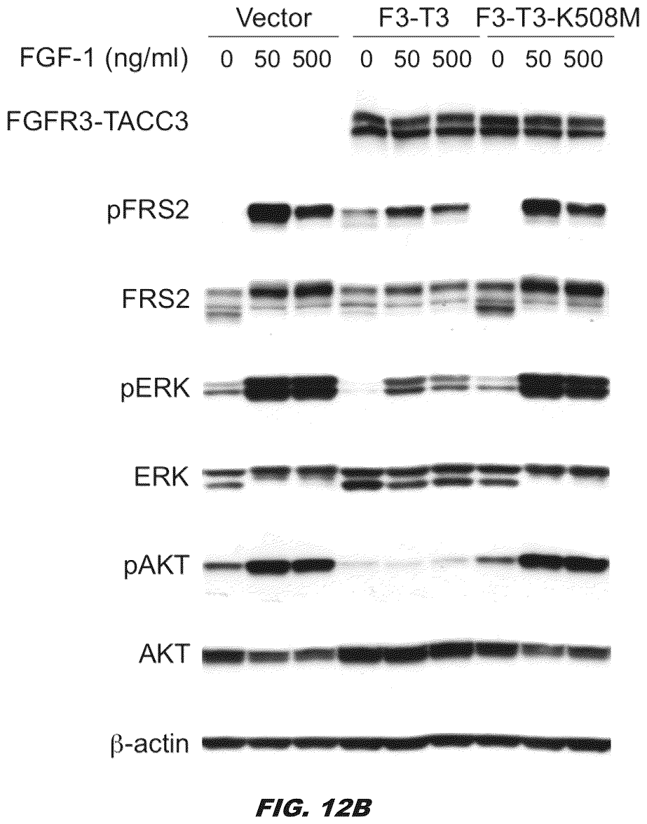

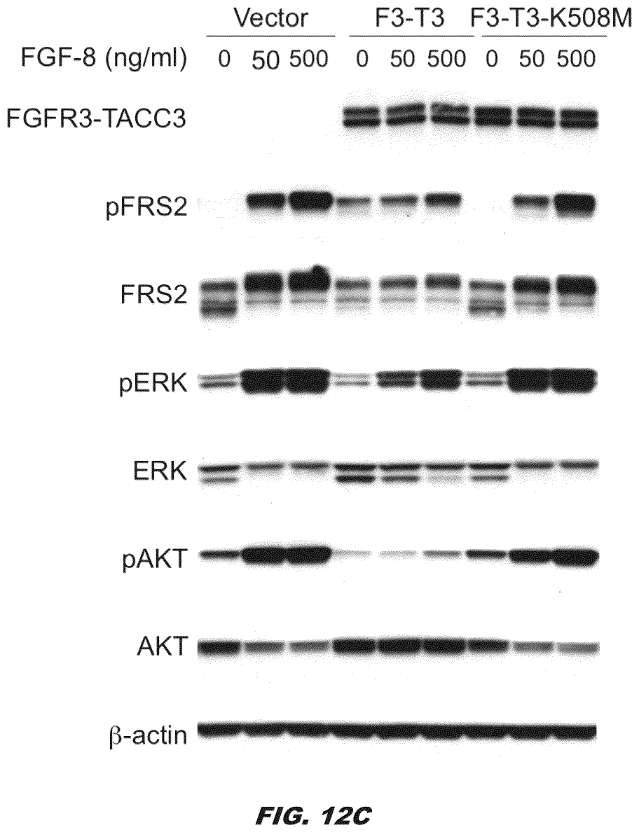

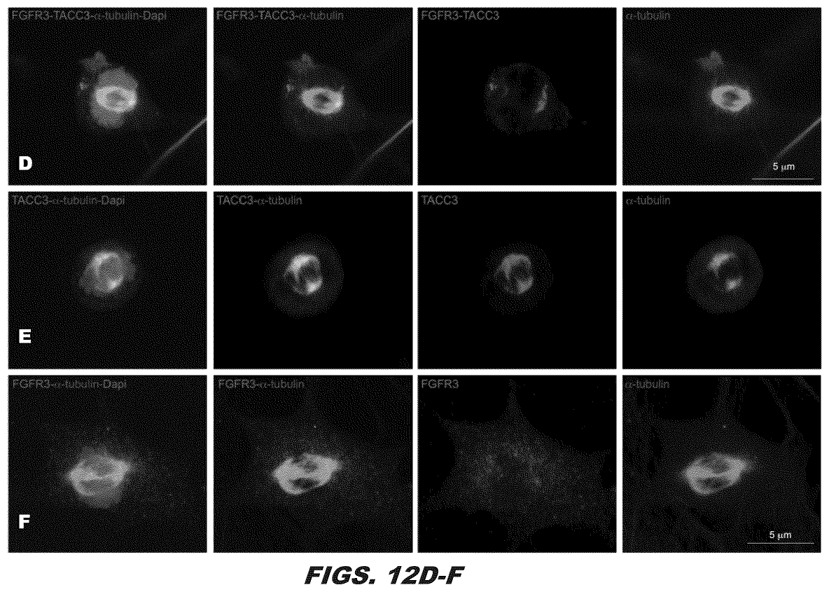

The FGFR-TACCfusions interfere with mitotic progression and induce chromosome missegregation and aneuploidy. To elucidate the mechanism by which the FGFR-TACC fusion drives oncogenesis, it was explored whether it activates downstream FGFR signaling. FGFR3-TACC3 failed to hyperactivate the canonical signaling events downstream of FGFR (pERK and pAKT) in the presence or absence of the ligands FGF-1, FGF-2 or FGF-8 (Wesche et al., 2011) (FIGS. 12A, 12B and 12C). However, FGFR3-TACC3 displayed constitutive phosphorylation of its TK domain and the adaptor protein FRS2, both of which were abolished by the specific inhibitor of FGFR-associated TK activity PD173074 (Mohammadi et al., 1998) or the K508M mutation (FIG. 4A). Thus, FGFR3-TACC3 gains constitutive kinase activity that is essential for oncogenic transformation but the downstream signaling of this aberrant activity is distinct from the canonical signaling events downstream to FGFR. By driving the localization of the fusion protein, the TACC domain can create entirely novel TK-dependent functions. The TACC domain is essential for the localization of TACC proteins to the mitotic spindle (Hood and Royle, 2011; Peset and Vernos, 2008). Confocal imaging showed that FGFR3-TACC3 designed an arc-shaped structure bending over and encasing the metaphase spindle poles, frequently displaying asymmetry towards one of the two poles and relocated to the midbody as cells progressed into the late stages of mitosis (telophase and cytokinesis) (FIGS. 4B and 12D). Conversely, the localization of TACC3 was restricted to spindle microtubules and did not relocalize to the midbody (FIG. 12E). Wild type FGFR3 lacked discrete localization patterns in mitosis (FIG. 12F).

The mitotic localization of FGFR3-TACC3 indicates that it may impact the fidelity of mitosis and perturb the accurate delivery of the diploid chromosomal content to daughter cells, thus generating aneuploidy. Mitotic progression of individual cells was examined in vector-transduced and FGFR3-TACC3 expressing cells co-expressing histone H2B-GFP by time-lapse microscopy. The average time from nuclear envelope breakdown to anaphase onset was increased in cells expressing FGFR3-TACC3 in comparison with control cells. The mitotic delay was further exacerbated by difficulties in completing cytokinesis (FIGS. 4C and 4D).

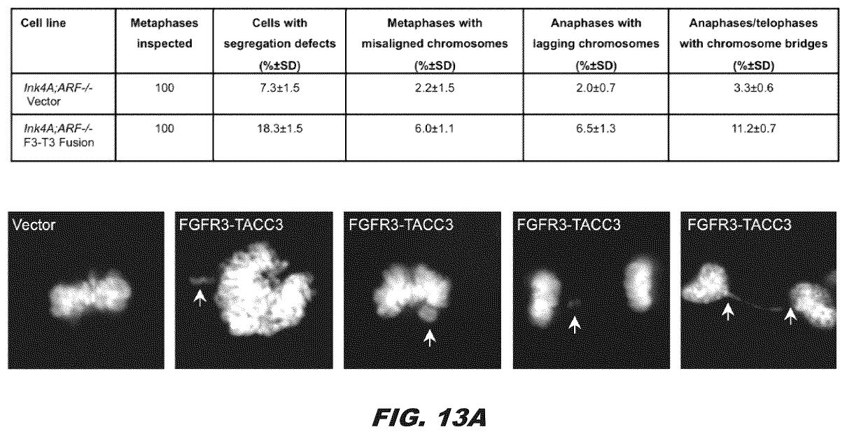

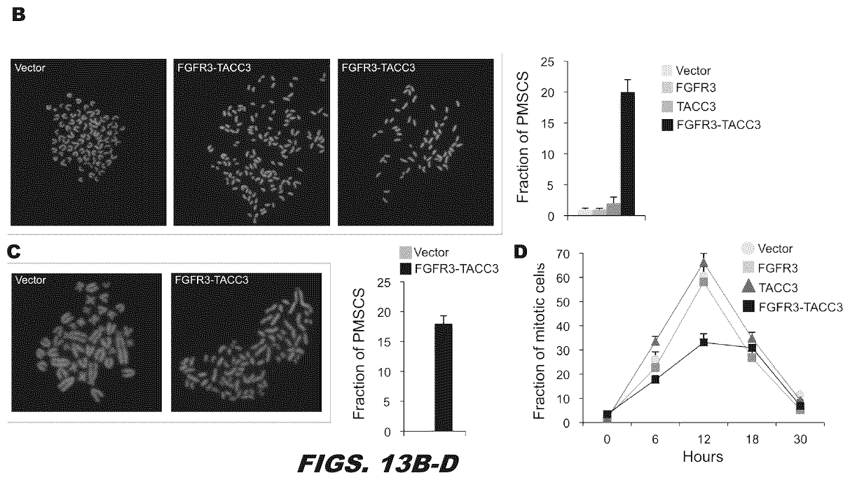

Next, it was determined whether the expressions of the FGFR-TACC fusion proteins induce defects of chromosomal segregation. Quantitative analyses of mitoses revealed that cells expressing FGFR3-TACC3 or FGFR1-TACC1 exhibit a three to five fold increase of chromosomal segregation errors than control cells. The most frequent mitotic aberrations triggered by the fusion proteins were misaligned chromosomes during metaphase, lagging chromosomes at anaphase and chromosome bridges that impaired cytokinesis and generated micronuclei in the daughter cells (FIGS. 4E, 4F and 13A). Aberrations at the metaphase-anaphase transition frequently lead to the inability of mitotic cells to maintain a metaphase arrest after treatment with a spindle poison. Over 18% of cells expressing FGFR3-TACC3 displayed prematurely separated sister chromatids in contrast with less than 3% in control, FGFR3 or TACC3-expressing cells (FIGS. 13B and 13C). Accordingly, cells expressing the fusion protein were unable to efficiently arrest in metaphase after nocodazole treatment (FIG. 13D).

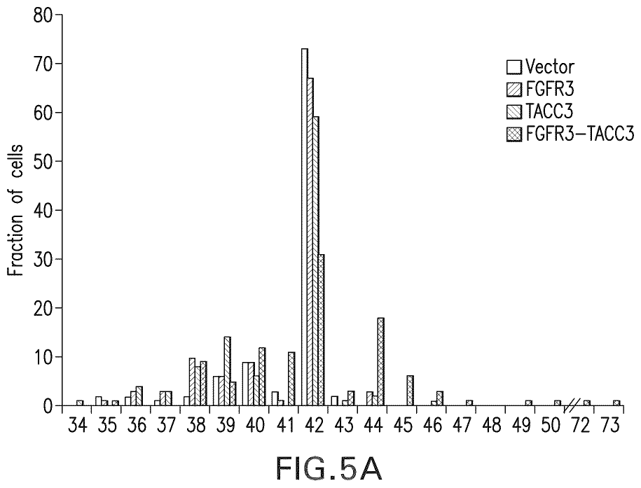

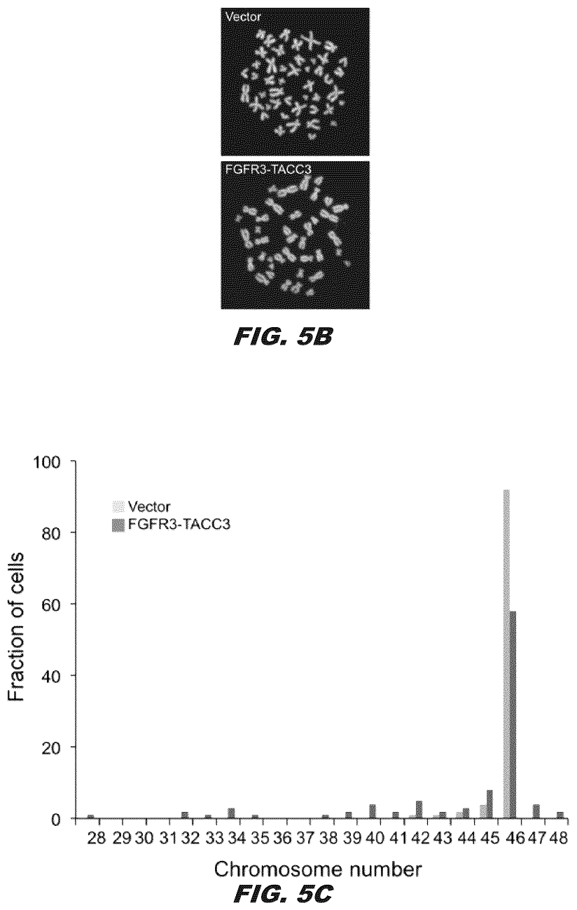

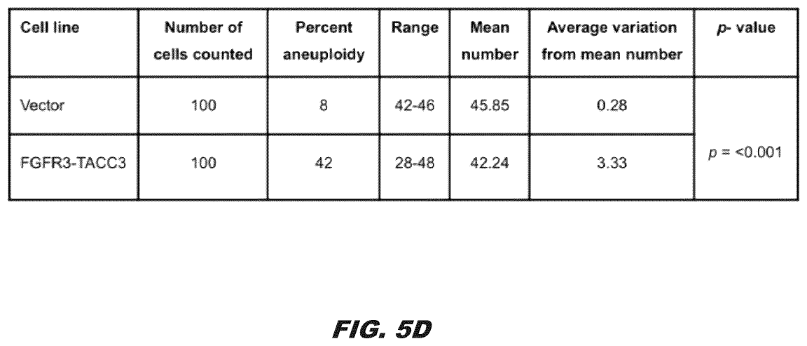

The above findings indicate that expression of the FGFR3-TACC3 fusion protein may spark aneuploidy. Karyotype analysis revealed that FGFR3-TACC3 increased over 2.5 fold the percent of aneuploidy and led to the accumulation of cells with broad distribution of chromosome counts in comparison with cells transduced with empty vector, FGFR3 or TACC3 (FIG. 5A). Accordingly, GSC-1123 contained aneuploid modal number of chromosomes (49) and manifested a broad distribution of chromosome counts characterized by 60% of metaphase spreads that deviate from the mode (Table 7)

TABLE 7
Chromosome analysis by SKY of 20 cells from the GSC-1123 culture

| Cell # | Chr # | +1 | +2 | +3 | t(3:14) | +4 | (−4) | del(4) | +5 | +6 | +7 | del(7) | +8 | −9 | +9 | −10 | +10 | +11 |
|---|---|---|---|---|---|---|---|---|---|---|---|---|---|---|---|---|---|---|
| 1 | 97 | 2 | 2 | 2 | | 2 | | 1 | 2 | 2 | 4 | | 2 | | 2 | | | 2 |
| 2 | 51 | | | | | | | | | | 1 | | | | | 1 | | |
| 3 | 49 | | | | | | | | | | 1 | | | | | 1 | | |
| 4 | 50 | | | | | | | | | | 1 | | | | | 1 | | |
| 5 | 49 | | | | | | | | | | 1 | | | | | 1 | | |
| 6 | 86 | 2 | 2 | 2 | | 2 | | | 2 | | 3 | | 1 | | 1 | | | 2 |
| 7 | 95 | 2 | 2 | 2 | | 2 | | | 1 | 2 | 4 | | 2 | | 2 | | | 2 |
| 8 | 98 | 2 | 3 | 2 | 1 | 2 | | | 1 | 2 | 4 | | 3 | | 1 | | | 2 |
| 9 | 86 | 2 | 1 | 1 | 1 | 1 | | | 1 | 1 | 6 | | 2 | | 3 | | 1 | 1 |
| 10 | 44 | | | 1 | 1 | | 1 | | | | 1 | 1 | | 1 | | 1 | | |
| 11 | 49 | | | | | | | | | | 1 | 1 | | | | 1 | | |
| 12 | 49 | | | | | | | | | | 1 | | | | | 1 | | |
| 13 | 98 | 2 | 2 | 2 | 2 | | | | 2 | 2 | 4 | | 2 | | 2 | | | 2 |
| 14 | 49 | | | | | | | | | | 1 | | | | | 1 | | |
| 15 | 48 | | | | | | | | | | 1 | | | | | 1 | | |
| 16 | 51 | | | | | | | | | | 1 | | | | | 1 | | |
| 17 | 49 | | | | | | | | | | 1 | | | | | 1 | | |
| 18 | 50 | | | | | | | | | | 1 | 1 | | | | 1 | | |
| 19 | 49 | | | | | | | | | | 1 | | | | | 1 | | |
| 20 | 49 | | | | | | | | | | 1 | | | | | 1 | | |

| Cell # | Chr # | +12 | +13 | del(13) | −14 | +14 | +15 | −16 | +16 | +17 | +18 | +19 | +20 | −21 | +21 | −22 | +22 | +X |
|---|---|---|---|---|---|---|---|---|---|---|---|---|---|---|---|---|---|---|
| 1 | 97 | 2 | 2 | 2 | | 2 | 2 | | 2 | 2 | 3 | 4 | 4 | | 2 | | 2 | 2 |
| 2 | 51 | | 2 | 2 | | | | | | | 1 | 1 | 1 | | | | | |
| 3 | 49 | | | 1 | | | | | | | 1 | 1 | 1 | | | | | |
| 4 | 50 | | | 1 | | | | | | | 1 | 1 | 1 | | | | | 1 |
| 5 | 49 | | | 1 | | | | | | | 1 | 1 | 1 | | | | | |
| 6 | 86 | 2 | 2 | 2 | | 2 | 2 | | | 2 | 4 | 3 | 3 | | 1 | | | 2 |
| 7 | 95 | 2 | 2 | 2 | | 2 | 2 | | 2 | 2 | 4 | 2 | 4 | | 2 | | 2 | 2 |
| 8 | 98 | 2 | 2 | 2 | | 2 | 2 | | 2 | 2 | 3 | 4 | 4 | | 3 | | 2 | 2 |
| 9 | 86 | 2 | 2 | 2 | | | | | 2 | 1 | 4 | 3 | 1 | | 2 | | 1 | 2 |
| 10 | 44 | | | | 1 | | | 1 | | | 1 | | 1 | | | 1 | | |
| 11 | 49 | | | | | | | | | | 1 | 1 | 1 | | | | | |
| 12 | 49 | | | 1 | | | | | | | 1 | 1 | 1 | | | | | |
| 13 | 98 | 2 | 2 | | | 2 | 2 | | 2 | 2 | 4 | 4 | 4 | | 2 | 2 | | 2 |
| 14 | 49 | | | 1 | | | | | | | 1 | 1 | 1 | | | | | |
| 15 | 48 | | | | | | | | | | 1 | 1 | 1 | 1 | | | | |
| 16 | 51 | | | 1 | | 1 | | | | | 1 | | | | | | 2 | 1 |
| 17 | 49 | | 1 | | | | | | | | 1 | 1 | 1 | | | | | |
| 18 | 50 | | 1 | 1 | | | | | | | 1 | 1 | 1 | | | | | |
| 19 | 49 | | | | | | | | | | 1 | 1 | 1 | | | | | |
| 20 | 49 | | | 1 | | | | | | | 1 | 1 | 1 | | | | | |

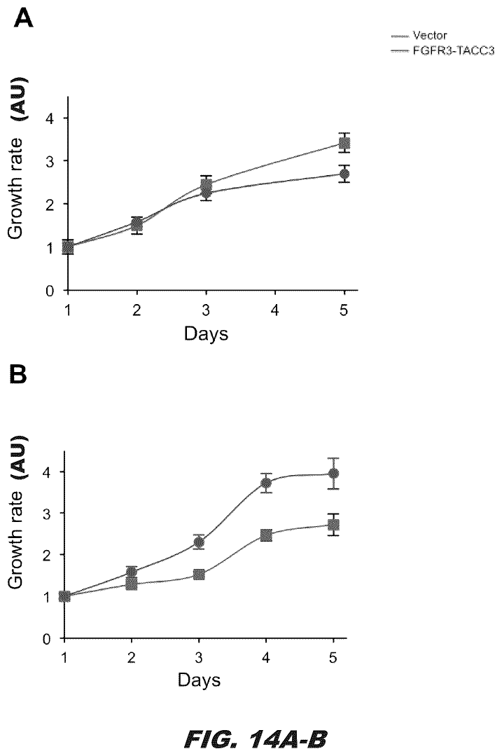

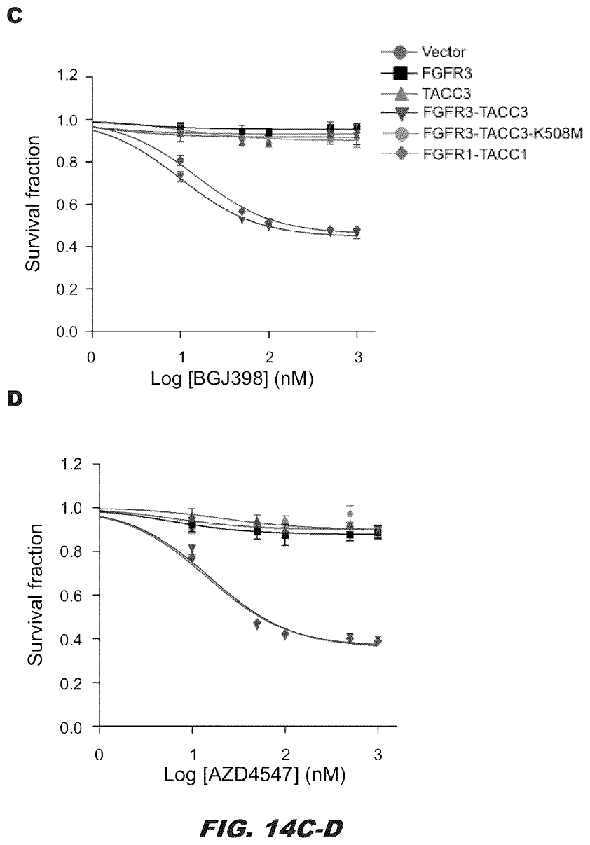

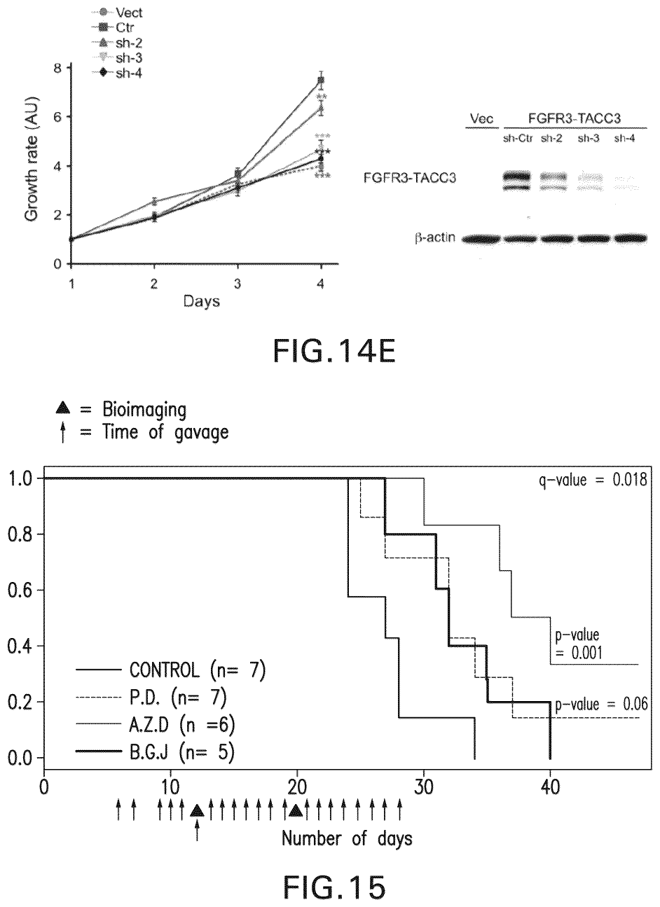

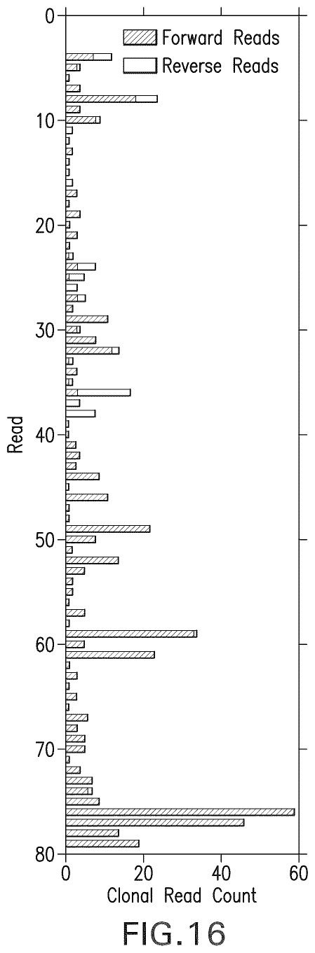

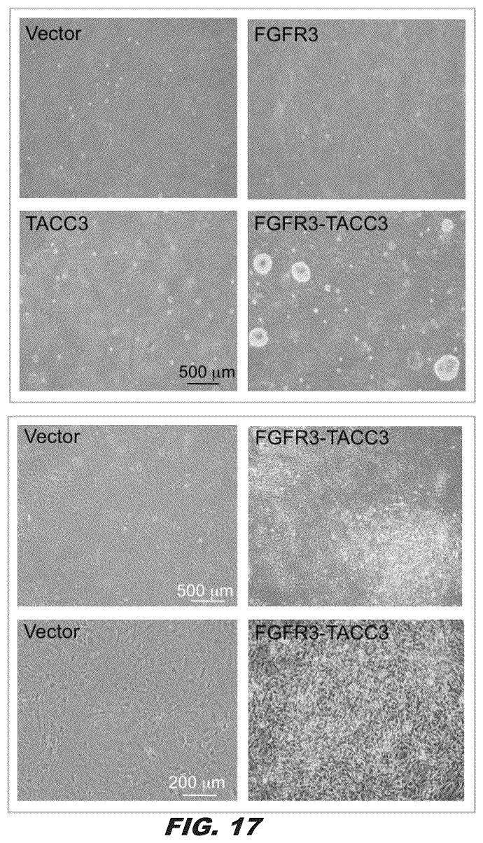

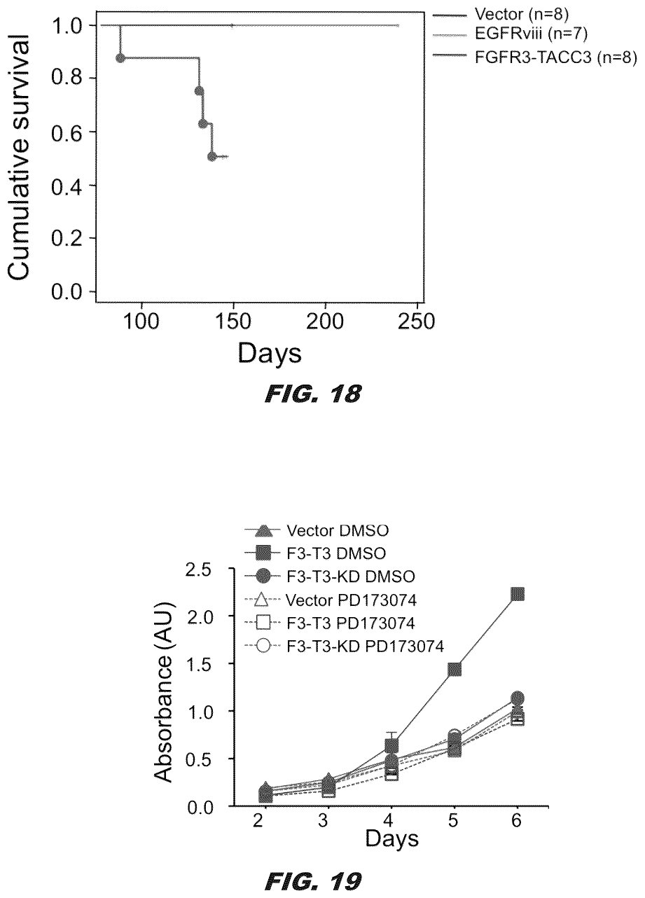

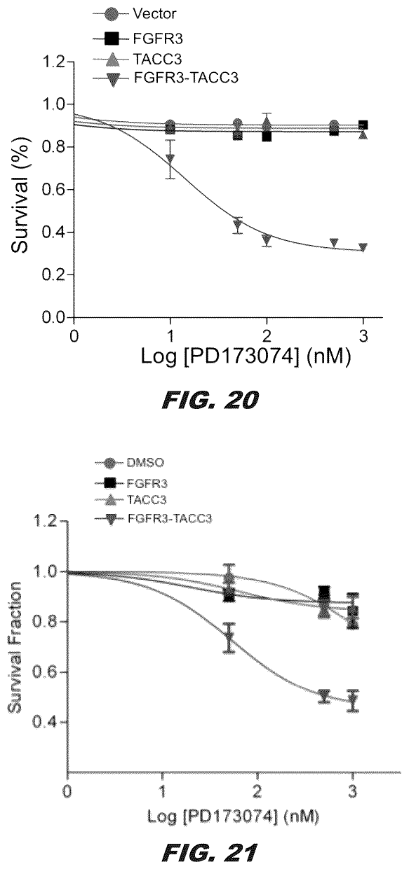

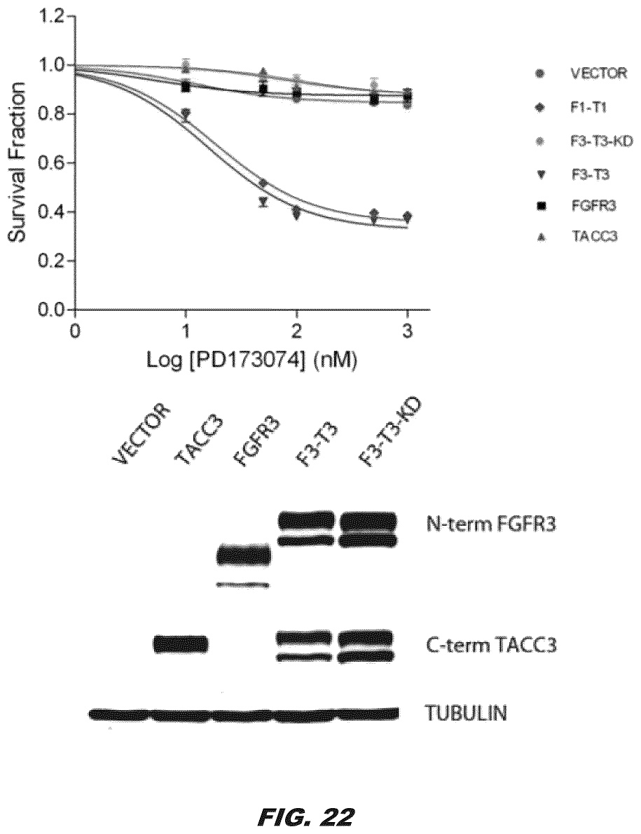

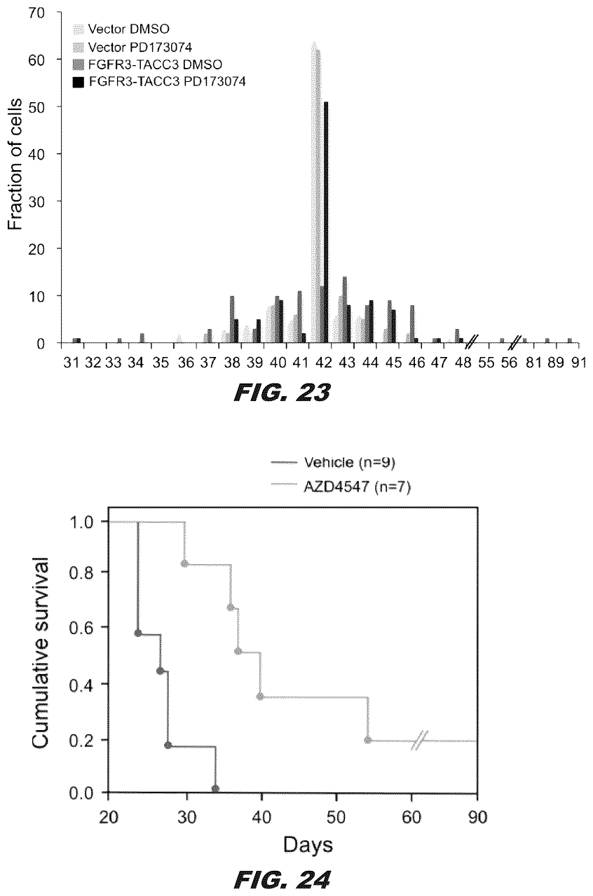

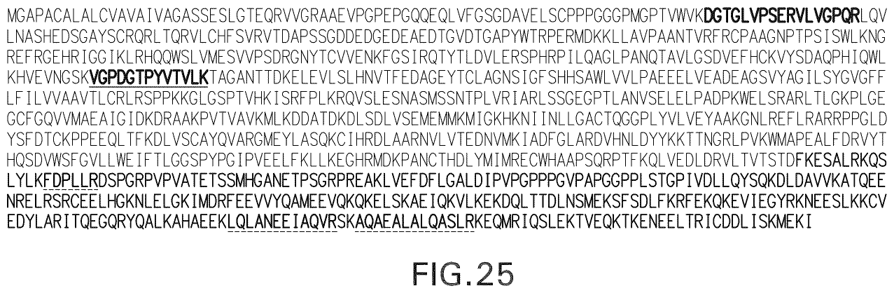

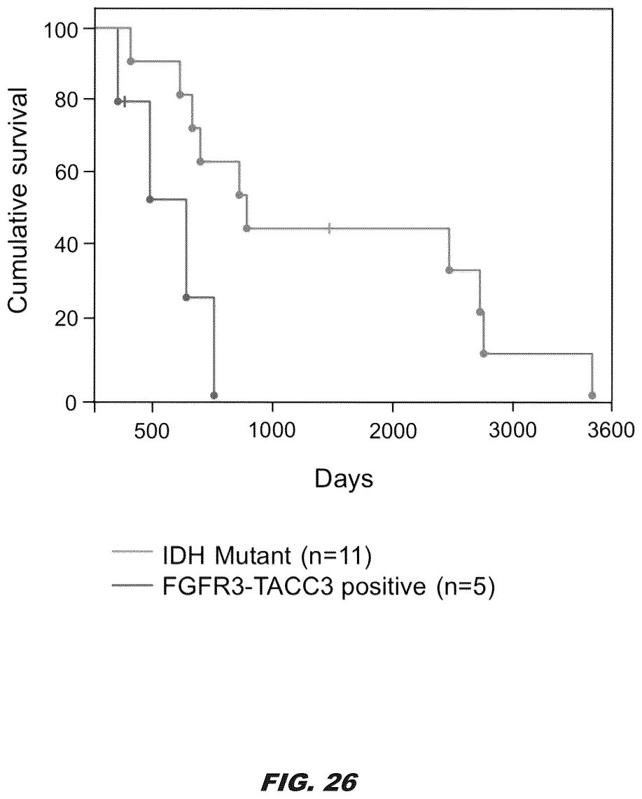

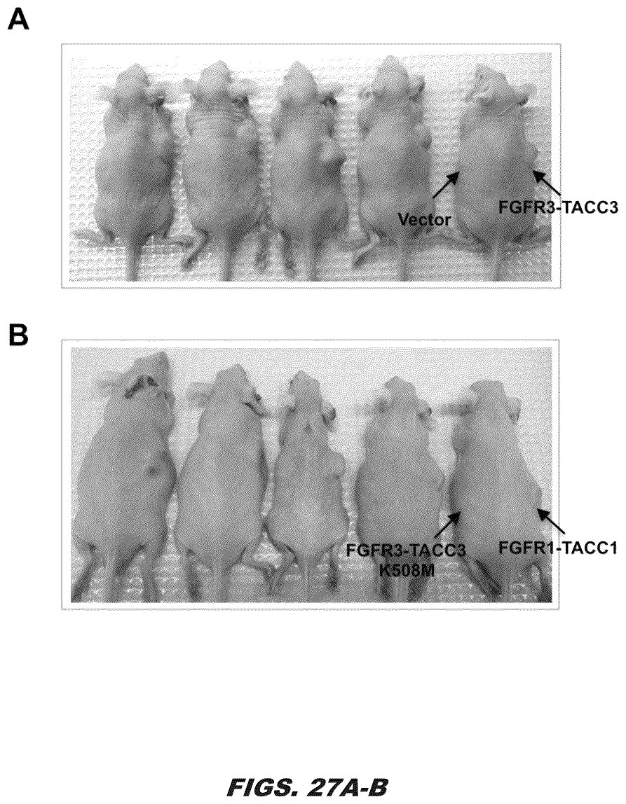

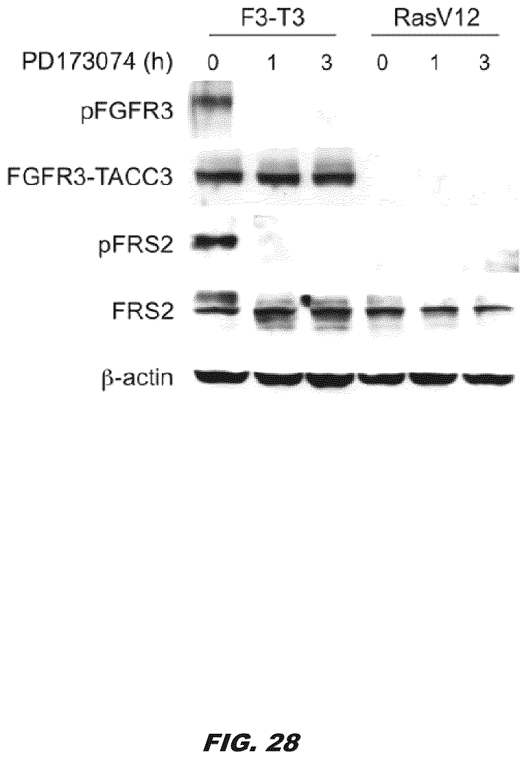

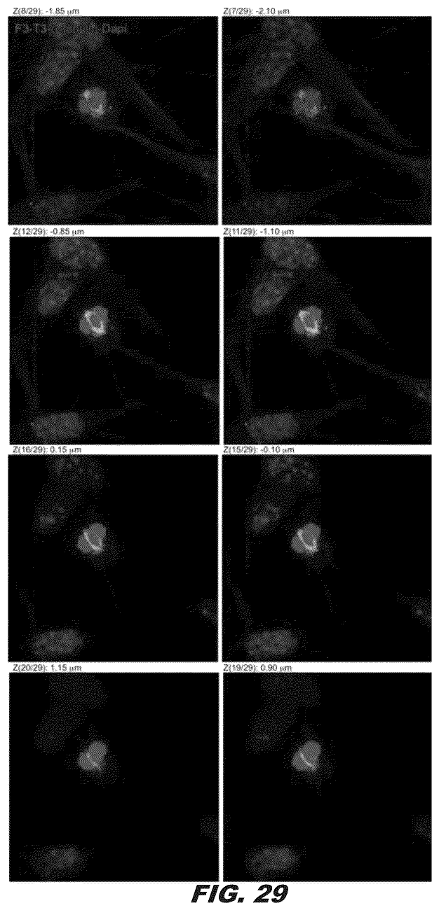

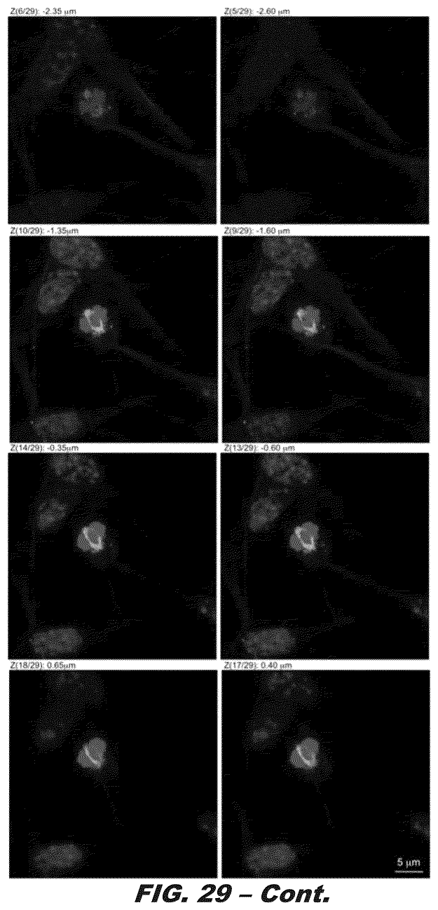

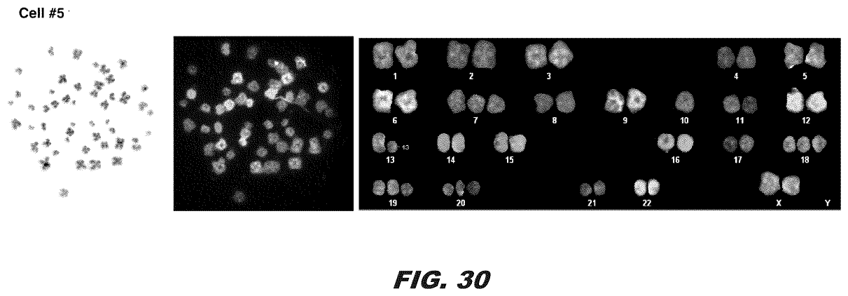

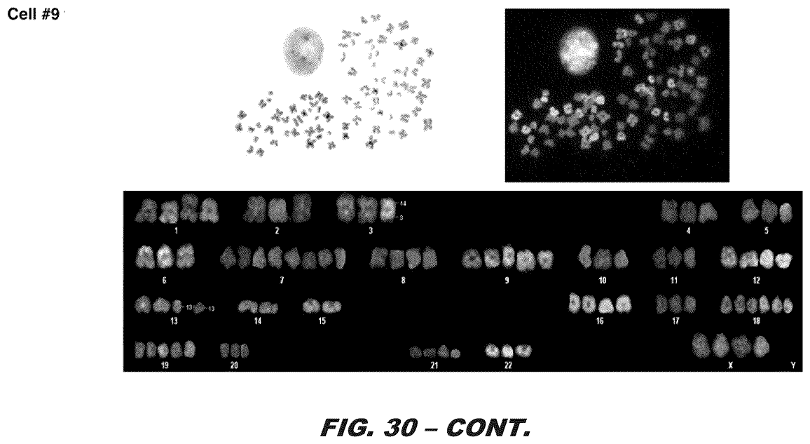

Next, it was determined whether aneuploidy is a direct consequence of FGFR3-TACC3 expression and is induced in human diploid neural cells. Primary human astrocytes analyzed six days after transduction with the FGFR3-TACC3 lentivirus exhibited a 5-fold increase of the rate of aneuploidy and a significantly wider distribution of chromosome counts (FIGS. 5B, 5C and SD)). Consistent that aneuploidy is detrimental to cellular fitness, acute expression of FGFR3-TACC3 compromised the proliferation capacity of human astrocytes. However, continuous culture of FGFR3-TACC3-expressing human astrocytes led to progressive gain of proliferative capacity that overrode that of control cells (FIG. 14A, 14B). Thus, the acute expression of FGFR3-TACC3 in primary normal human cells from the central nervous system causes CIN and aneuploidy with an acute fitness cost manifested by slower proliferation.

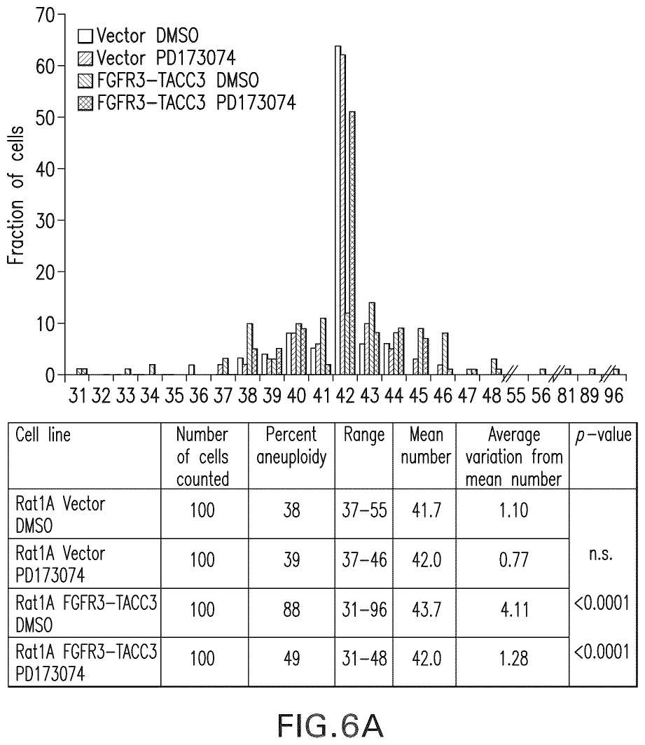

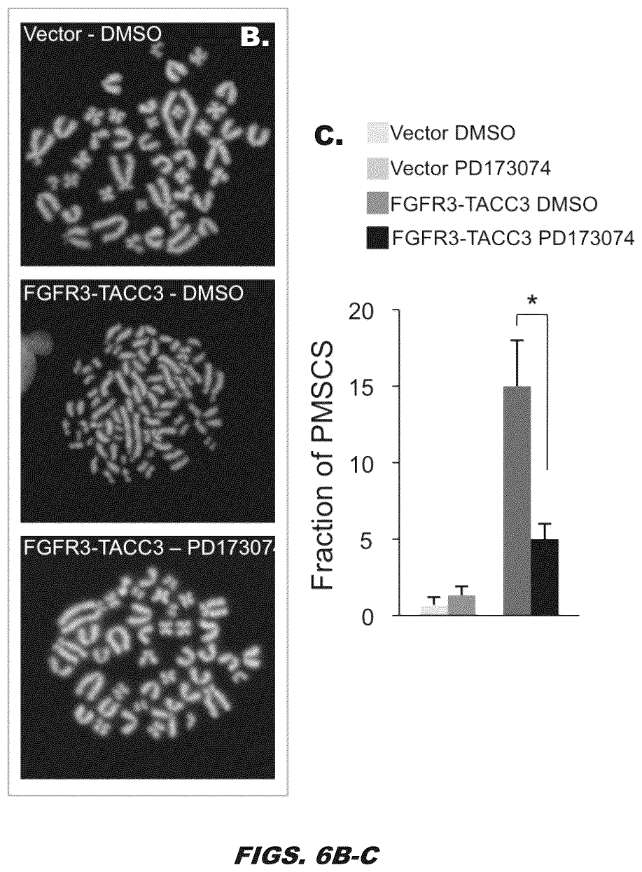

It was also determined whether the CIN and aneuploidy caused by FGFR3-TACC3 requires the TK activity of FGFR3 and can be corrected. Treatment with PD173074 rescued the aneuploidy caused by FGFR3-TACC3 by over 80%, restored the narrow distribution of chromosome counts typical of control cells and largely corrected the cohesion defect (FIGS. 6A, 6B and 6C). Together, these findings indicate that the CIN and aneuploidy caused by rearrangements of FGFR and TACC genes are reversible and suggest that specific FGFR kinase inhibition may be a valuable therapeutic strategy in tumor cells expressing FGFR-TACC fusion proteins.

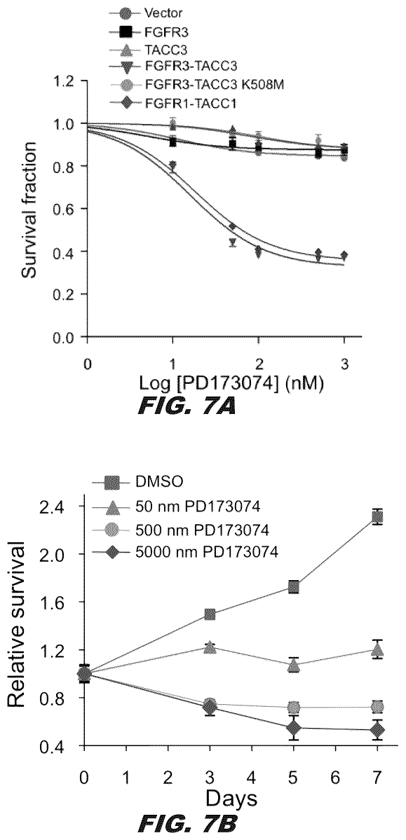

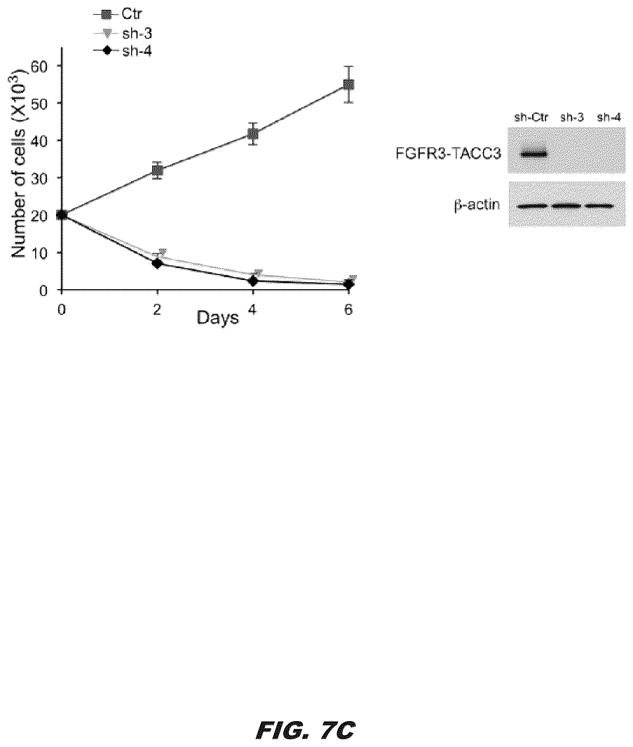

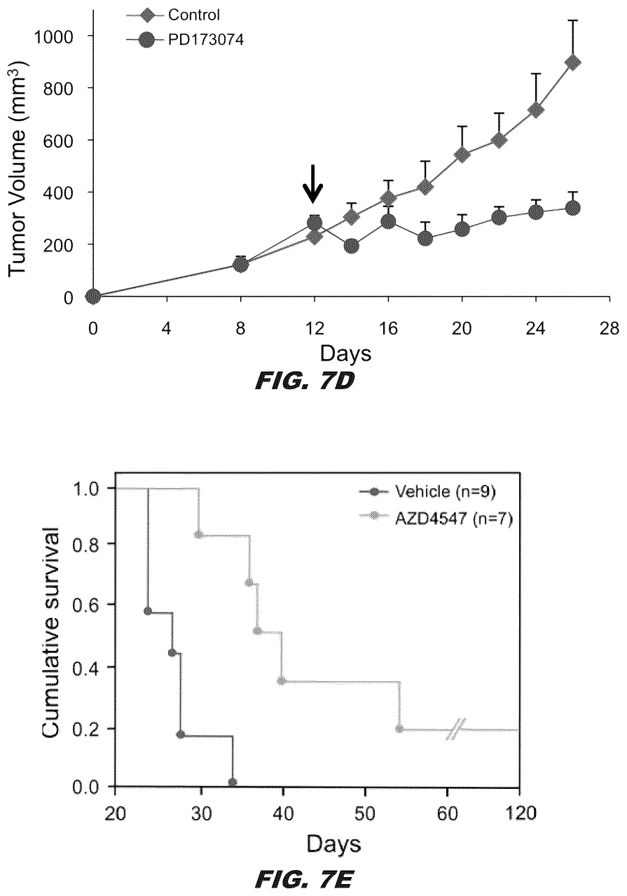

FGFR-TACC fusion proteins are new therapeutic targets in GBM. Driver genetic alterations trigger a state of oncogene addiction in the cancer cells harboring them that can be exploited therapeutically. To ask whether FGFR-TACC fusions confer addiction to FGFR-TK activity, cell growth was analyzed in the presence of PD173074, AZD4547 or BGJ398, the latter being two highly specific inhibitors of FGFR-TK under clinical investigation (Gavine et al., 2012; Guagnano et al., 2011). Each of the three drugs inhibited growth of cells expressing FGFR3-TACC3 and FGFR1-TACC1 at concentrations <10 nM whereas they were ineffective at concentrations as high as 1 μM in cells transduced with vector, FGFR3, TACC3 and the FGFR3-TACC3-K508M mutant (FIGS. 7A, 14C and 14D). These findings underscore the elevated degree of specificity for FGFR kinase inhibition towards cells carrying the fusion protein. The growth of GSC-1123 cells, which naturally harbor the FGFR3-TACC3 translocation, was also abolished by nanomolar concentrations of FGFR-TK inhibitors (FIG. 7B). Targeting of the fusion gene by FGFR3 shRNA inhibited the growth of cells ectopically expressing FGFR3-TACC3 and GSC-1123 proportionally to the silencing efficiency of FGFR3-TACC3 (FIGS. 7C and 14E).

Finally, it was determined whether treatment with PD173074 of mice bearing glioma xenografts of FGFR3-TACC3 transformed astrocytes inhibits tumor growth. Twelve days after injection of tumor cells, subcutaneous tumors were present in all animals. The mice were randomized in two cohorts and treated with PD173074 or vehicle. PD173074 elicited a potent growth inhibition of FGFR3-TACC3 glioma (FIG. 7D). To confirm the efficacy of a clinically meaningful FGFR-TK inhibitor using a more anatomically relevant model, the AZD4547 FGFR inhibitor, a compound under clinical investigation (Gavine et al., 2012), was used against intracranial luciferase-expressing FGFR3-TACC3-driven glioma xenografts. After an engraftment period, tumor-bearing animals were treated with either AZD4547 or vehicle. Oral administration of AZD4547 markedly prolonged survival (FIG. 7E). Taken together, the data provide a strong rationale for a clinical trial based on FGFR inhibitors in GBM harboring FGFR-TACC rearrangements.

### Discussion

This work has established that recurrent, oncogenic and addicting gene fusions identify a subset of GBM patients. The functional characterization of FGFR-TACC fusions indicates that the constitutively active FGFR-TK and the TACC domain of the fusion protein are both essential for oncogenesis. The TACC-dependent mis-localization to mitotic cells of the FGFR kinase results in aberrant compartmentalization of a constitutively active TK to the mitotic spindle pole, thus providing a mechanistic explanation for the impaired mitotic fidelity, chromosome mis-segregation and aneuploidy instigated by the fusion protein.

Without being bound by theory, mutation of the genes that control chromosome segregation during mitosis can explain the high rate of CIN and aneuploidy, which is typical of most solid tumors including GBM (Gordon et al., 2012). A few examples of mutational inactivation of candidate genes have been reported in human cancer (Solomon et al., 2011; Thompson et al., 2010). However, gain-of-function mutations causally implicated in the control of mitotic fidelity have not been described. This clashes with the classical observation from cell fusion experiments that the underlying mechanisms that cause CIN behave as dominant traits, indicating that the CIN phenotype results from gain-of-function events rather than gene inactivation (Lengauer et al., 1997, 1998). The FGFR-TACC gene fusion is a novel mechanism for the initiation of CIN and provides a clue to the nature of dominant mutations responsible for aneuploidy in human cancer.

The rapid emergence of mitotic defects and aneuploid cell populations triggered by the fusion protein in normal human astrocytes, combined with the correction of aneuploidy after short inhibition of FGFR-TK activity indicate that aneuploidy is a key event in tumor induction by the FGFR-TACC gene fusions. Induction of aneuploidy per se is detrimental to cellular fitness (Sheltzer and Amon, 2011). Full-blown tumorigenesis requires cooperation between aneuploidy and genetic lesions that confer growth advantage and protect cells against the detrimental effects of aneuploidy (Coschi and Dick, 2012; Holland and Cleveland, 2009; Weaver and Cleveland, 2009). Therefore, the potent tumor-initiating activity of FGFR-TACC fusions shows that the novel oncoproteins have growth-promoting signaling functions that complement the loss of mitotic fidelity with ensuing karyotypic alterations (Sheltzer and Amon, 2011).

Targeted therapies against common genetic alterations in GBM have not changed the dismal clinical outcome of the disease, most likely because they have systematically failed to eradicate the truly addicting oncoprotein activities of GBM. The highly specific anti-tumor effects and the correction of aneuploidy precipitated by FGFR-TK inhibition of FGFR-TACC-driven GBM provide a strong rationale for clinical trials based on FGFR inhibitors in patients harboring FGFR-TACC rearrangements. The computational gene fusion discovery pipeline reported here detected other GBM cases in which FGFR family genes are implicated in additional gene fusions beyond the FGFR-TACC rearrangements. Therefore, the frequency of 3.1% is likely to be an underestimate of the target GBM patient population that may benefit from FGFR-TK inhibition.

### EXPERIMENTAL PROCEDURES

Cell culture and isolation and maintenance of GSCs. Rat1A, mouse astrocytes Ink4A;Arf−/−, and human astrocytes were cultured in DMEM supplemented with 10% FBS. Isolation and culture of GSCs was performed as described (Carro et al., 2010). For treatment in vitro with PD173074, AZD4547 or BJG398, cells infected with vector control, FGFR3, TACC3, FGFR-TACC fusions or FGFR3-TACC3-K508M were seeded in 96-well plates and treated with increasing concentrations of FGFR inhibitors. After 72-120 h, growth rate was measured using the 3-(4,5-dimethythiazol-2-yl)-2,5-diphenyl tetrazolium bromide (MTT) assay. Data were expressed as mean±SD. Proliferation rate in GSC-1123 infected with FGFR3 shRNA lentivirus was determined by plating dissociated gliomaspheres at 2×10⁴ cells/well in twelve-well plates 5 days after infection. The number of viable cells was determined by trypan blue exclusion in triplicate cultures obtained from triplicate independent infections. Cell number was scored every other day.

DNA, RNA Preparation, Genomic and Real-Time Quantitative PCR (qRT-PCR).

The validation of fusion transcripts was performed using both genomic and RT-PCR with forward and reverse primer combinations designed within the margins of the paired-end read sequences detected by RNA-seq. DNA, RNA preparation and qRT-PCR were performed as described (Carro et al., 2010; Zhao et al., 2008). To identify novel fusion transcripts within the GBM cohort, PCR primers pairs were designed to bind upstream to the TK domain of the FGFR genes and inside or downstream the Coiled Coil domain of the TACC genes. Expressed fusion transcript variants were subjected to direct sequencing to confirm sequence and frame. Primer sequences are included below.

Subcutaneous xenografts and drug treatment. Rat1A or Ink4A;Arf−/− astrocytes (5×10⁵) transduced with different lentiviral constructs were suspended in 150 μl of PBS, together with 30 μl of Matrigel (BD Biosciences), and injected subcutaneously in the flank of athymic nude (Nu/Nu) mice (Charles River Laboratories, Wilmington, MA). For experiments with FGFR inhibitors, mice carrying ˜200-300 mm³ subcutaneous tumors derived from Ink4A;Arf−/− astrocytes were randomized to receive 50 mg/kg PD173074 in 0.05 M lactate buffer (pH 5) or an equal volume of lactate buffer by oral gavage. Treatment was administered for three cycles consisting of four consecutive days followed by two days of rest. Tumor diameters were measured with caliper, and tumor volumes estimated using the formula: 0.5×length×width². Data are expressed as mean±SE. Mice were sacrificed when tumors in the control group reached the maximal size allowed.

Orthotopic transplantation and drug treatment. Ink4A;Arf−/− astrocytes carrying a luciferase expressing vector were transduced with FGFR3-TACC3 lentivitus. 1×10³ cells in 2 pl of saline were injected in the caudate-putamen of 4-6 week old male athymic nude (Nu/Nu) mice using a stereotaxic frame (coordinates relative to bregma: 0.5 mm anterior; 1.1 mm lateral; 3.0 mm ventral) and a 26 gauge Hamilton syringe. Six days after injection, mice underwent bioluminescence imaging using a Xenogen CCD apparatus and were randomized to receive 50 mg/kg AZD4547 in 1% Tween 80 (treatment group) or DMSO in an equal volume of vehicle by oral gavage (control group). AZD4547 was administered daily for two cycles of 10 days with a two day interval. Mice were monitored daily and sacrificed when neurological symptoms appeared. Kaplan-Meier survival curve was generated using the DNA Statview software package (AbacusConcepts, Berkeley, CA). Log-rank analysis was performed on the Kaplan-Meier survival curve to determine statistical significance.

Intracranial injections of lentiviruses. Intracranial injection of FGFR3-TACC3-shp53, EGFRvIII-shp53 or shp53 pTomo lentiviruses was performed in 4 week-old C57/BL/6J mice in accordance with guidelines of IACUC Committee. Briefly, 1.8 μl of purified lentiviral particles in PBS (1×10⁹/ml) were injected into the dentate gyrus using a stereotaxic frame (coordinates relative to bregma: 1.45 mm posterior; 1.65 mm lateral; 2.4 mm ventral) and a 26 gauge Hamilton syringe. Mice were monitored daily and sacrificed when neurological symptoms appeared. Mouse brain was analyzed histopathologically and by immunofluorescence staining.

Histology and immunostaining. Tissue preparation and immunohistochemistry on brain tumors and immunofluorescence staining were performed as previously described (Carro et al., 2010; Zhao et al., 2009; Zhao et al., 2008). Antibodies used in immunostaining and immunoblotting are listed below.

Cloning and Lentiviralproduction. Lentivirus preparation and infections were performed as described (Carro et al., 2010) and are detailed in Extended Experimental Procedures.

Karyotype analysis. Cultured cells were colcemid (20 ng/ml) treated for 90 minutes before harvesting for karyotopic analysis as detailed in Extended Experimental procedures. At least one hundred cells in metaphase were examined for chromosome count. PMSCS was scored in cells where a majority of the sister chromosomes were no longer associated. Two-tailed unpaired t-tests with Welch's correction were performed for comparison of means analysis.

Immunofluorescence and live-cell microscopy. Immunofluorescence microscopy was performed on cells fixed with 4% PFA in PHEM (60 mM Pipes, 27 mM Hepes, 10 mM EGTA, 4 mM MgSO₄, pH 7.0). Cells were permeabilized using 1% Triton X-100. Mitotic spindles were visualized by anti-α-tubulin antibody (Sigma). Secondary antibodies conjugated to Alexa Fluor-488/-594 (Molecular Probes) were used. All staining with multiple antibodies were performed in a sequential manner. DNA was stained by DAPI (Sigma). Fluorescence microscopy was performed on a Nikon AIR MP microscope.

Identification of gene fusions from whole transcriptome (RNA-seq) and exome sequencing. RNA-Sequencing was performed from total RNA extracted from GSC cultures isolated from nine GBM patients using Illumina HiSeq 2000, producing roughly 60.3 million paired reads per sample. Using the global alignment software Burrows-Wheeler Aligner (BWA) (Li and Durbin, 2009) with modified Mott's trimming, an initial seed length of 32, maximum edit distance of 2 and a maximum gap number of 1, on average 43.1 million reads were mapped properly to the RefSeq transcriptome and, of the remaining, 8.6 million were mapped to the hg19 genome per sample. The remaining 14.3% of paired reads including those that failed to map to either transcriptome or genome with proper forward-reverse (F-R) orientation, within expected insert size, and with minimal soft clipping (unmapped portions at the ends of a read)—were considered to be appropriate for gene fusion analysis.

A novel computational pipeline was constructed called TX-Fuse that identifies two sources of evidence for the presence of a gene fusion: 1. Split inserts, in which each read of a mate pair maps entirely to one side of a breakpoint, and 2. Individual split reads that span a breakpoint. Split inserts are readily detected from BWA mapping. On the other hand, split reads demand precision alignment of smaller nucleotide stretches. To that end, the pipeline employs the local alignment package BLAST with word size of 20, identity cutoff of 95%, expectation cutoff of 104, and soft filtering to map raw paired reads against the RefSeq transcriptome. From this procedure, a list of potential split reads were obtained that were filtered to ensure maintenance of coding frame in the predicted fusion transcript given the proper F-R orientation in the read pair. False positive candidates produced from paralogous gene pairs were also screened out using the Duplicated Genes Database and the EnsemblCompara GeneTrees (Vilella et al., 2009). Pseudogenes in the candidate list were annotated using the list from HUGO Gene Nomenclature Committee (HGNC) database (Seal et al., 2011) and given lower priority. For each remaining gene fusion candidate, a virtual reference was created based on the predicted fusion transcript and re-mapped all unmapped reads using BLAST with word size of 16, identity cutoff of 85%, query coverage greater than 85%, and expectation cutoff of 10′ to obtain a final count of split reads and inserts. Moreover, sequencing depth per base of the virtual reference was calculated to corroborate that components of each gene participating in the gene fusion were highly expressed.

To establish the recurrence of the initial panel of gene fusion candidates, the gene fusion discovery pipeline was modified to produce EXome-Fuse, which probes for fusions within the available dataset of paired-read exome DNA sequencing of 84 matched GBM samples from TCGA. To increase sensitivity for gene fusion identification, reads unmapped by BWA were aligned to the gene pair participating in each fusion candidate using a BLAST word size of 24 for split inserts and 16 for split read and split insert discovery. Given that the breakpoint detected in DNA cannot directly indicate the resulting breakpoint in the transcribed RNA, no restriction was made on split insert orientation. For split reads, it was only required that the component of the split read mapped to the same gene as its mate maintained F-R directionality.

Co-outlier expression and CNV analysis from TCGA GBM samples. Tomlins et al. (Tomlins et al., 2005) reported that outlier gene expression from microarray datasets identifies candidate oncogenic gene fusions. Wang et al. (Wang et al., 2009) suggested a “breakpoint principle” for intragenic copy number aberrations in fusion partners. The two principles (outlier expression and intragenic CNV) were combined to identify candidate gene fusions in GBM samples from Atlas-TCGA. Genomic and expression data sets were downloaded from TCGA public data portal as available on Dec. 1, 2011, where a description of TCGA data types, platforms, and analyses is also available (2008). Specific data sources were (according to Data Levels and Data Types) as follows: Expression data, “Level 2” normalized signals per probe set (Affymetrix HT_HG-U133A) of 84 samples; Copy number data, “Level 1” raw signals per probe (Affymetrix Genome-Wide Human SNP Array 6.0) of the 4 FGFR3-TACC3 gene fusion positive samples (tumor and matched normal control).

The gene expression analysis was performed first using R³. The median absolute deviation (MAD) was calculated and then a gene was labeled as an outlier according to the following formula: Z_{i,j}=0.6745(x_{i,j}−mean(xᵢ))/MADᵢ>3.5 (Iglewicz and Hoaglin, 1993). Samples were identified as ECFS (expression candidate fusion sample) if both genes of interest (e. g. FGFR3 and TACC3) displayed outlier behavior (co-outliers). Next, ECFS were analyzed for CNV using pennCNV (Wang et al., 2007). Tumors samples were paired to their normal controls to obtain the log ratio values and the VEGA algorithm was used to obtain a more accurate segmentation (Morganella et al., 2010).

Karyotypic Analysis. The colcemid treated cells were trypsinized, centrifuged for 7 minutes at 200×g, and the cell pellet re-suspended in warmed hypotonic solution and incubated at 37° C. for 13 minutes. The swollen cells were then centrifuged and the pellet re-suspended in 8 ml of Carnoy's fixative (3:1 methanol: glacial acetic acid). The cell suspension was centrifuged and washed twice in Carnoy's fixative. After the last centrifugation, the cells were resuspended in 0.5 to 1 ml of freshly prepared fixative to produce an opalescent cell suspension. Drops of the final cell suspension were placed on clean slides and air-dried. Slides were stained with DAPI and metaphases were analyzed under a fluorescent microscope.

Cloning and Lentiviral production. Lentiviral expression vectors, pLOC-GFP (Open Biosystems) and pTomo-shp53, were used to clone FGFR3, TACC3, FGFR3-TACC3, FGFR3-TACC3-K508M, and FGFR1-TACC1. pTomo-shp53 was a gift of Inder Verma and Dinorah Friedman-Morvinski (Salk Institute, San Diego). The FGFR3-TACC3-K508M mutant was generated using the Phusion Site Direct Mutagenesis kit (NEB, USA). MISSION shRNAs clones (pLKO.1 lentiviral expression vectors) against FGFR3 were purchased from Sigma. The hairpin sequences targeting the FGFR3 gene are—

(#TRCN0000000372; Sh#2)
(SEQ ID NO: 182)
5′-TGCGTCGTGGAGAACAAGTTT-3′;
(#TRCN0000430673; Sh#3)
(SEQ ID NO: 183)
5′-GTTCCACTGCAAGGTGTACAG-3′;
(#TRCN0000000374; Sh#4)
(SEQ ID NO: 184)
5′-GCACAACCTCGACTACTACAA-3′.

Genomic and mRNA RT-PCR. Total RNA was extracted from cells by using RNeasy Mini Kit (QIAGEN), following the manufacturer instructions. 500 ng of total RNA was retro-transcribed by using the Superscript III kit (Invitrogen), following the manufacturer instructions. The cDNAs obtained after the retro-transcription was used as templates for qPCR. The reaction was performed with a Roche480 thermal cycler, by using the Absolute Blue QPCR SYBR Green Mix from Thermo Scientific. The relative amount of specific mRNA was normalized to 18S. Results are presented as the mean±SD of triplicate amplifications.

Primers used are:

hFGFR3-RT-FW1:
(SEQ ID NO: 162)
5′-GTAACCTGCGGGAGTTTCTG-3′;
hFGFR3-RT-REV1:
(SEQ ID NO: 163)
5′-ACACCAGGTCCTTGAAGGTG-3′;
hTACC3-RT-FW2:
(SEQ ID NO: 164)
5′-CCTGAGGGACAGTCCTGGTA-3′;
hTACC3-RT-REV2:
(SEQ ID NO: 165)
5′-AGTGCTCCCAAGAAATCGAA-3′;
hWRAP53-RT-FW1:
(SEQ ID NO: 180)
5′-AGAGGTGACCACCAATCAGC-3′;
hWRAP53-RT-REV1:
(SEQ ID NO: 181)
5′-CGTGTCCCACACAGAGACAG-3′.

Primers used for the screening of FGFR-TACC fusions are:

FGFR3-FW1:
(SEQ ID NO: 166)
5′-CGTGAAGATGCTGAAAGACGATG-3′;
TACC3-REV1:
(SEQ ID NO: 167)
5′-AAACGCTTGAAGAGGTCGGAG-3′;
FGFR1-FW1:
(SEQ ID NO: 168)
5′-ATGCTAGCAGGGGTCTCTGA-3′;
TACC1-REV1:
(SEQ ID NO: 169)
5′-CCCTTCCAGAACACCTTTCA-3′.

Primers used for genomic detection of FGFR3-TACC3 fusion in GBM-1123 and GSC-1123 are:

Genomic FGFR3-FW1:
(SEQ ID NO: 170)
5′-ATGATCATGCGGGAGTGC-3′;
genomicTACC3-REV1:
(SEQ ID NO: 171)
5′-GGGGGTCGAACTTGAGGTAT-3′.

Primers used to validate fusions detected by RNA-seq are:

POLR2A-FW1:
(SEQ ID NO: 172)
5′-CGCAGGCTTTTTGTAGTGAG-3′;
WRAP53-REV1:
(SEQ ID NO: 173)
5′-TGTAGGCGCGAAAGGAAG-3′;
PIGU-FW1:
(SEQ ID NO: 174)
5′-GAACTCATCCGGACCCCTAT-3′;
NCOA6-REV1:
(SEQ ID NO: 175)
5′-GCTTTCCCCATTGCACTTTA-3′;
ST8SIA4-FW1:
(SEQ ID NO: 176)
5′-GAGGAGAGAAGCACGTGGAG-3′;
PAM-REV1:
(SEQ ID NO: 177)
5′-GGCAGACGTGTGAGGTGTAA-3′;
CAPZB-FW:
(SEQ ID NO: 178)
5′-GTGATCAGCAGCTGGACTGT-3′;
UBR4-REV1:
(SEQ ID NO: 179)
5′-GAGCCTGGGCATGGATCT-3′.

Confocal microscopy imaging. For immunofluorescence of fixed cells, images were recorded with a Z-optical spacing of 0.25 m using a Nikon A1R MP and a 60×1.3 oil objective and analyzed using ImageJ software (National Institute of Health). For live-cell analyses, Rat1A cells infected with pLNCX-H2B retrovirus and transduced with lentiviral vector or FGFR3-TACC3 fusion were seeded in glass bottom dishes in phenol red free DMEM and followed by time-lapse microscopy using the Nikon AIR MP biostation at 37° C. and 5% CO₂/95% air. Images with a Z-optical spacing of 1 m were recorded every 4 min for 8 h. Images of unchallenged mitosis from early prophase until cytokinesis were processed using ImageJ software (National Institute of Health). The time-point of nuclear envelope breakdown (NEB) was defined as the first frame showing loss of smooth appearance of chromatin and anaphase was the first frame when chromosome movement towards the poles became apparent. Nuclear envelope reconstitution (NER) was defined as the first frame showing nuclei decondensation.

Box and whisker plots were calculated from image sequences from at least 50 recorded cells. Two-tailed unpaired t-tests with Welch's correction were performed for comparison of means analysis using StatView software (AbacusConcepts, Berkeley, CA).

Immunofluorescence. Antibodies and concentrations used in immunofluorescence staining are:

| | | | |
|---|---|---|---|
| Anti-Ki67 | Rabbit | 1:1000 | Vector Labs |
| Anti-pHH3 | Rabbit | 1:500 | Millipore |
| Anti-FGFR3 | Mouse | 1:1000 | Santa Cruz |
| Anti-Tacc3 | Goat | 1:1000 | USBiological |
| Anti-a-tubulin | Mouse | 1:1000 | Sigma |
| Anti-Nestin | Mouse | 1:1000 | BD Pharmingen |
| Anti-Olig2 | Rabbit | 1:200 | IBL |
| Anti-GFAP | Rabbit | 1:200 | Dako |
| Anti-ERK | Rabbit | 1:1000 | Cell Signaling |
| Anti-pERK | Rabbit | 1:1000 | Cell Signaling |
| AntiFRS | Rabbit | 1:250 | Santa Cruz |
| Anti-pFRS | Rabbit | 1:1000 | Cell Signaling |
| Anti-AKT | Rabbit | 1:1000 | Cell Signaling |
| Anti-pAKT473 | Rabbit | 1:1000 | Cell Signaling |

### REFERENCES

Ablain, J., Nasr, R., Bazarbachi, A., and de The, H. (2011). The Drug-Induced Degradation of Oncoproteins: An Unexpected Achilles' Heel of Cancer Cells? Cancer Discov. 1, 117-127.
Bass, A. J., Lawrence, M. S., Brace, L. E., Ramos, A. H., Drier, Y., Cibulskis, K., Sougnez, C., Voet, D., Saksena, G., Sivachenko, A., et al. (2011). Genomic sequencing of colorectal adenocarcinomas identifies a recurrent VTI1A-TCF7L2 fusion. Nat. Genet. 43, 964-968.
Cahill, D. P., Kinzler, K. W., Vogelstein, B., and Lengauer, C. (1999). Genetic instability and darwinian selection in tumours. Trends Cell. Biol. 9, M57-60.
Carro, M. S., Lim, W. K., Alvarez, M. J., Bollo, R. J., Zhao, X., Snyder, E. Y., Sulman, E. P., Anne, S. L., Doetsch, F., Colman, H., et al. (2010). The transcriptional network for mesenchymal transformation of brain tumours. Nature 463, 318-325.
Coschi, C. H., and Dick, F. A. (2012). Chromosome instability and deregulated proliferation: an unavoidable duo. Cell. Mol. Life Sci. 69, 2009-2024 Druker, B. J. (2009). Perspectives on the development of imatinib and the future of cancer research. Nat. Med. 15, 1149-1152.
Furnari, F. B., Fenton, T., Bachoo, R. M., Mukasa, A., Stommel, J. M., Stegh, A., Hahn, W. C., Ligon, K. L., Louis, D. N., Brennan, C., et al. (2007). Malignant astrocytic glioma: genetics, biology, and paths to treatment. Genes Dev. 21, 2683-2710.
Gavine, P. R., Mooney, L., Kilgour, E., Thomas, A. P., Al-Kadhimi, K., Beck, S., Rooney, C., Coleman, T., Baker, D., Mellor, M. J., et al. (2012). AZD4547: An Orally Bioavailable, Potent, and Selective Inhibitor of the Fibroblast Growth Factor Receptor Tyrosine Kinase Family. Cancer Res. 72, 2045-2056.
Gerber, D. E., and Minna, J. D. (2010). ALK inhibition for non-small cell lung cancer: from discovery to therapy in record time. Cancer Cell 18, 548-551.
Gordon, D. J., Resio, B., and Pellman, D. (2012). Causes and consequences of aneuploidy in cancer. Nature reviews Genet. 13, 189-203.
Guagnano, V., Furet, P., Spanka, C., Bordas, V., Le Douget, M., Stamm, C., Brueggen, J., Jensen, M. R., Schnell, C., Schmid, H., et al. (2011). Discovery of 3-(2,6-dichloro-3,5-dimethoxy-phenyl)-1-{6-[4-(4-ethyl-piperazin-1-yl)-phenylamino]-pyrimidin-4-yl}-1-methyl-urea (NVP-BGJ398), a potent and selective inhibitor of the fibroblast growth factor receptor family of receptor tyrosine kinase. J. Med. Chem. 54, 7066-7083.
Holland, A. J., and Cleveland, D. W. (2009). Boveri revisited: chromosomal instability, aneuploidy and tumorigenesis. Nat. Rev. Mol. Cell. Biol. 10, 478-487.
Hood, F. E., and Royle, S. J. (2011). Pulling it together: The mitotic function of TACC3. Bioarchitecture 1, 105-109.
Lee, J., Kotliarova, S., Kotliarov, Y., Li, A., Su, Q., Donin, N. M., Pastorino, S., Purow, B. W., Christopher, N., Zhang, W., et al. (2006). Tumor stem cells derived from glioblastomas cultured in bFGF and EGF more closely mirror the phenotype and genotype of primary tumors than do serum-cultured cell lines. Cancer Cell 9, 391-403.
Lengauer, C., Kinzler, K. W., and Vogelstein, B. (1997). Genetic instability in colorectal cancers. Nature 386, 623-627.
Lengauer, C., Kinzler, K. W., and Vogelstein, B. (1998). Genetic instabilities in human cancers. Nature 396, 643-649.
Lo, H. W. (2010). EGFR-targeted therapy in malignant glioma: novel aspects and mechanisms of drug resistance. Curr. Mol. Pharmacol. 3, 37-52.
Marumoto, T., Tashiro, A., Friedmann-Morvinski, D., Scadeng, M., Soda, Y., Gage, F. H., and Verma, I. M. (2009). Development of a novel mouse glioma model using lentiviral vectors. Nat. Med. 15, 110-116.
Mitelman, F., Johansson, B., and Mertens, F. (2007). The impact of translocations and gene fusions on cancer causation. Nat. Rev. Cancer 7, 233-245.
Mohammadi, M., Froum, S., Hamby, J. M., Schroeder, M. C., Panek, R. L., Lu, G. H., Eliseenkova, A. V., Green, D., Schlessinger, J., and Hubbard, S. R. (1998). Crystal structure of an angiogenesis inhibitor bound to the FGF receptor tyrosine kinase domain. EMBO J. 17, 5896-5904.
Ohgaki, H., and Kleihues, P. (2005). Population-based studies on incidence, survival rates, and genetic alterations in astrocytic and oligodendroglial gliomas. J. Neuropathol. Exp. Neurol. 64, 479-489.
Peset, I., and Vernos, I. (2008). The TACC proteins: TACC-ling microtubule dynamics and centrosome function. Trends Cell. Biol. 18, 379-388.
Prensner, J. R., and Chinnaiyan, A. M. (2009). Oncogenic gene fusions in epithelial carcinomas. Curr Opin Genet. Dev. 19, 82-91.
Reardon, D. A., Desjardins, A., Vredenburgh, J. J., Gururangan, S., Friedman, A. H., Herndon, J. E., 2nd, Marcello, J., Norfleet, J. A., McLendon, R. E., Sampson, J. H., et al. (2010). Phase 2 trial of erlotinib plus sirolimus in adults with recurrent glioblastoma. J. Neurooncol. 96, 219-230.
Sheltzer, J. M., and Amon, A. (2011). The aneuploidy paradox: costs and benefits of an incorrect karyotype. Trends Genet. 27, 446-453.
Solomon, D. A., Kim, T., Diaz-Martinez, L. A., Fair, J., Elkahloun, A. G., Harris, B. T., Toretsky, J. A., Rosenberg, S. A., Shukla, N., Ladanyi, M., et al. (2011). Mutational inactivation of STAG2 causes aneuploidy in human cancer. Science 333, 1039-1043.
Stephens, P. J., McBride, D. J., Lin, M. L., Varela, I., Pleasance, E. D., Simpson, J. T., Stebbings, L. A., Leroy, C., Edkins, S., Mudie, L. J., et al. (2009). Complex landscapes of somatic rearrangement in human breast cancer genomes. Nature 462, 1005-1010.
Still, I. H., Vince, P., and Cowell, J. K. (1999). The third member of the transforming acidic coiled coil-containing gene family, TACC3, maps in 4p16, close to translocation breakpoints in multiple myeloma, and is upregulated in various cancer cell lines. Genomics 58, 165-170.
Thompson, S. L., Bakhoum, S. F., and Compton, D. A. (2010). Mechanisms of chromosomal instability. Curr. Biol. 20, R285-295.
Tomlins, S. A., Laxman, B., Dhanasekaran, S. M., Helgeson, B. E., Cao, X., Morris, D. S., Menon, A., Jing, X., Cao, Q., Han, B., et al. (2007). Distinct classes of chromosomal rearrangements create oncogenic ETS gene fusions in prostate cancer. Nature 448, 595-599.
Tomlins, S. A., Rhodes, D. R., Perner, S., Dhanasekaran, S. M., Mehra, R., Sun, X. W., Varambally, S., Cao, X., Tchinda, J., Kuefer, R., et al. (2005). Recurrent fusion of TMPRSS2 and ETS transcription factor genes in prostate cancer. Science 310, 644-648.
Turner, N., and Grose, R. (2010). Fibroblast growth factor signalling: from development to cancer. Nat. Rev. Cancer 10, 116-129.
van den Bent, M. J., Brandes, A. A., Rampling, R., Kouwenhoven, M. C., Kros, J. M., Carpentier, A. F., Clement, P. M., Frenay, M., Campone, M., Baurain, J. F., et al. (2009). Randomized phase II trial of erlotinib versus temozolomide or carmustine in recurrent glioblastoma: EORTC brain tumor group study 26034. J. Clin. Oncol. 27, 1268-1274.
Wang, X. S., Prensner, J. R., Chen, G., Cao, Q., Han, B., Dhanasekaran, S. M., Ponnala, R., Cao, X., Varambally, S., Thomas, D. G., et al. (2009). An integrative approach to reveal driver gene fusions from paired-end sequencing data in cancer. Nat. Biotechnol. 27, 1005-1011.
Weaver, B. A., and Cleveland, D. W. (2009). The role of aneuploidy in promoting and suppressing tumors. J. Cell. Biol. 185, 935-937.
Wesche, J., Haglund, K., and Haugsten, E. M. (2011). Fibroblast growth factors and their receptors in cancer. Biochem. J. 437, 199-213.
Yan, H., Parsons, D. W., Jin, G., McLendon, R., Rasheed, B. A., Yuan, W., Kos, I., Batinic-Haberle, I., Jones, S., Riggins, G. J., et al. (2009). IDH1 and IDH2 mutations in gliomas. New Engl. J. Med. 360, 765-773.
Zhao, X., D, D. A., Lim, W. K., Brahmachary, M., Carro, M. S., Ludwig, T., Cardo, C. C., Guillemot, F., Aldape, K., Califano, A., et al. (2009). The N-Myc-DLL3 cascade is suppressed by the ubiquitin ligase Huwel to inhibit proliferation and promote neurogenesis in the developing brain. Dev. Cell 17, 210-221.
Zhao, X., Heng, J. I., Guardavaccaro, D., Jiang, R., Pagano, M., Guillemot, F., Iavarone, A., and Lasorella, A. (2008). The HECT-domain ubiquitin ligase Huwel controls neural differentiation and proliferation by destabilizing the N-Myc oncoprotein. Nat Cell Biol 10, 643-653.
(2008). Comprehensive genomic characterization defines human glioblastoma genes and core pathways. Nature 455, 1061-1068.
Iglewicz, B., and Hoaglin, D. C. (1993). How to detect and handle outliers (Milwaukee, Wis.: ASQC).
Li, H., and Durbin, R. (2009). Fast and accurate short read alignment with Burrows-Wheeler transform. Bioinformatics 25, 1754-1760.
Morganella, S., Cerulo, L., Viglietto, G., and Ceccarelli, M. (2010). VEGA: variational segmentation for copy number detection. Bioinformatics 26, 3020-3027.
Seal, R. L., Gordon, S. M., Lush, M. J., Wright, M. W., and Bruford, E. A. (2011). genenames.org: the HGNC resources in 2011. Nucleic Acids Res 39, D514-519.
Tomlins, S. A., Rhodes, D. R., Perner, S., Dhanasekaran, S. M., Mehra, R., Sun, X. W., Varambally, S., Cao, X., Tchinda, J., Kuefer, R., et al. (2005). Recurrent fusion of TMPRSS2 and ETS transcription factor genes in prostate cancer. Science 310, 644-648.
Vilella, A. J., Severin, J., Ureta-Vidal, A., Heng, L., Durbin, R., and Birney, E. (2009). EnsemblCompara GeneTrees: Complete, duplication-aware phylogenetic trees in vertebrates. Genome Res. 19, 327-335.
Wang, K., Li, M., Hadley, D., Liu, R., Glessner, J., Grant, S. F., Hakonarson, H., and Bucan, M. (2007). PennCNV: an integrated hidden Markov model designed for high-resolution copy number variation detection in whole-genome SNP genotyping data. Genome Res. 17, 1665-1674.
Wang, X. S., Prensner, J. R., Chen, G., Cao, Q., Han, B., Dhanasekaran, S. M., Ponnala, R., Cao, X., Varambally, S., Thomas, D. G., et al. (2009). An integrative approach to reveal driver gene fusions from paired-end sequencing data in cancer. Nat. Biotechnol. 27, 1005-1011.

### Example 2—Fusions in GBM

TABLE 8
Soft agar colony assay

| Cell line | Vector | FGFR3 | TACC3 | F1-T1 Fusion | F3-T3 Fusion | F3-T3-K508M Fusion |
|---|---|---|---|---|---|---|
| Rat1 | 0 | 0 | 0 | 225.3 ± 10.0 | 198.7 ± 8.0 | 0 |
| Balb 3T3 | 0 | 0 | 0 | n.d. | 45.5 ± 8.9 | n.d. |

n.d.: not done

TABLE 9
Subcutaneous tumor xenografts

| Cell line | Vector | FGFR3 | TACC3 | F1-T1 Fusion | F3-T3 Fusion | F3-T3-K508M Fusion |
|---|---|---|---|---|---|---|
| Rat1 | 0/5 | 0/5 | 0/5 | n.d. | 5/5 | n.d. |
| Ink4A/Arf−/− | 0/9 | 0/5 | 0/5 | 8/8 | 12/12 | 0/8 |
| Astrocytes | | | | | | |

n.d.: not done

TABLE 10
Analysis of chromosomal number in Rat1 cells

| | Number of cells | Percent | | Mean | Average variation | |
|---|---|---|---|---|---|---|
| Cell line | counted | aneuploidy | Range | number | from mean number | p- value |
| Rat1A Vector | 100 | 27 | 35-43 | 41.2 | 1.2 | |
| Rat1A FGFR3 | 100 | 33 | 35-44 | 42.1 | 1.3 | n.s. |
| Rat1A TACC3 | 100 | 41 | 34-46 | 40.7 | 1.1 | n.s. |
| Rat1A FGFR3-TACC3 | 100 | 69 | 35-73 | 43.8 | 3.1 | <0.0001 |

TABLE 11
Analysis of chromosomal number in human astrocytes

| | Number of | Percent | | Mean | Average variation | |
|---|---|---|---|---|---|---|
| Cell line | cells counted | aneuploidy | Range | number | from mean number | p- value |
| Human Astrocytes Vector | 100 | 8 | 42-46 | 45.85 | 0.28 | p = <0.001 |
| Human Astrocytes FGFR3-TACC3 | 100 | 42 | 28-48 | 42.24 | 3.33 | |

### Example 3—Fusions in Other Cancers

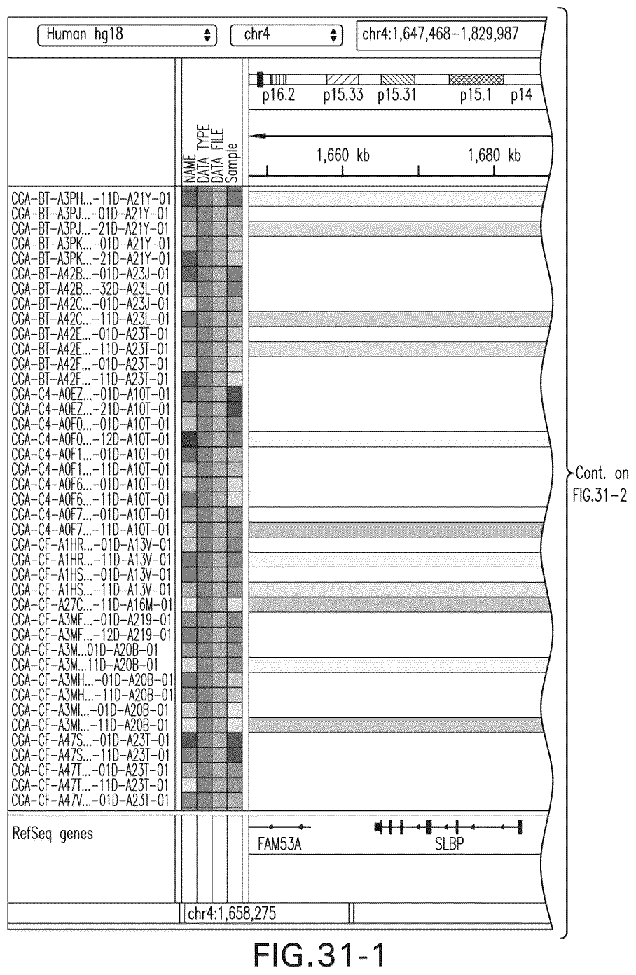

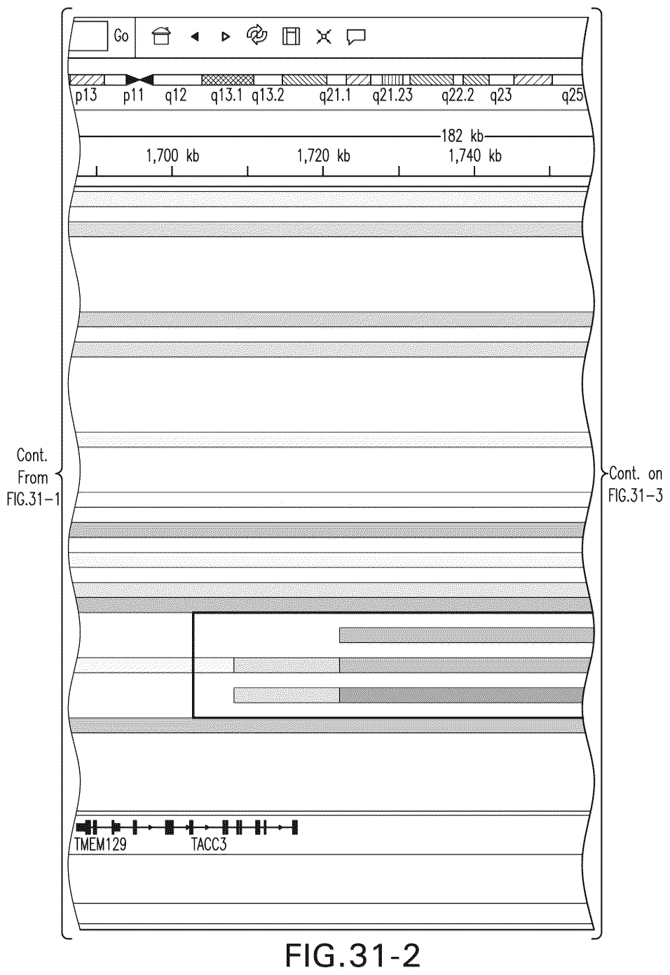

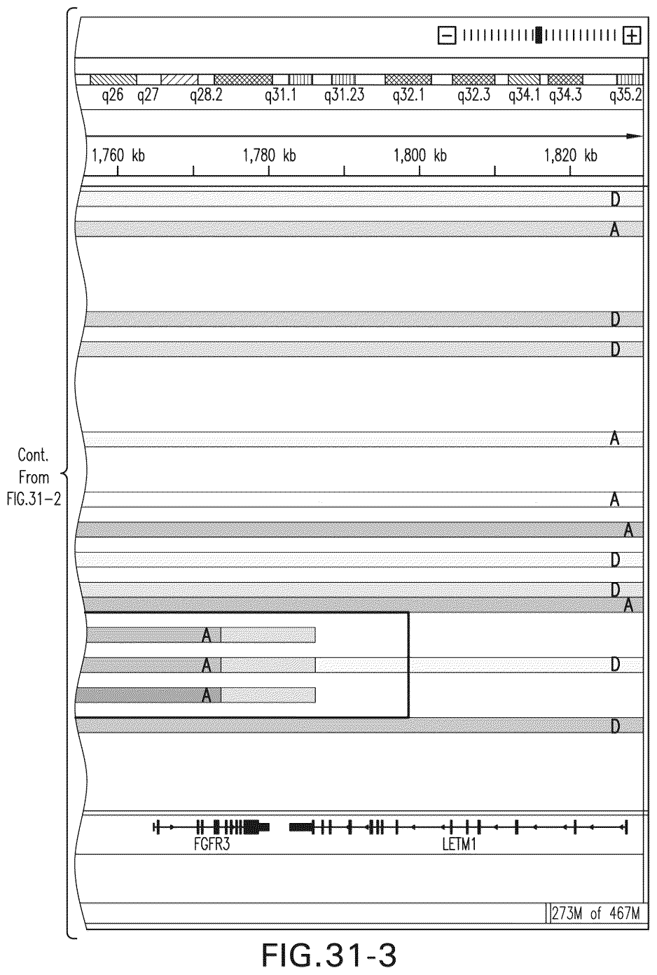

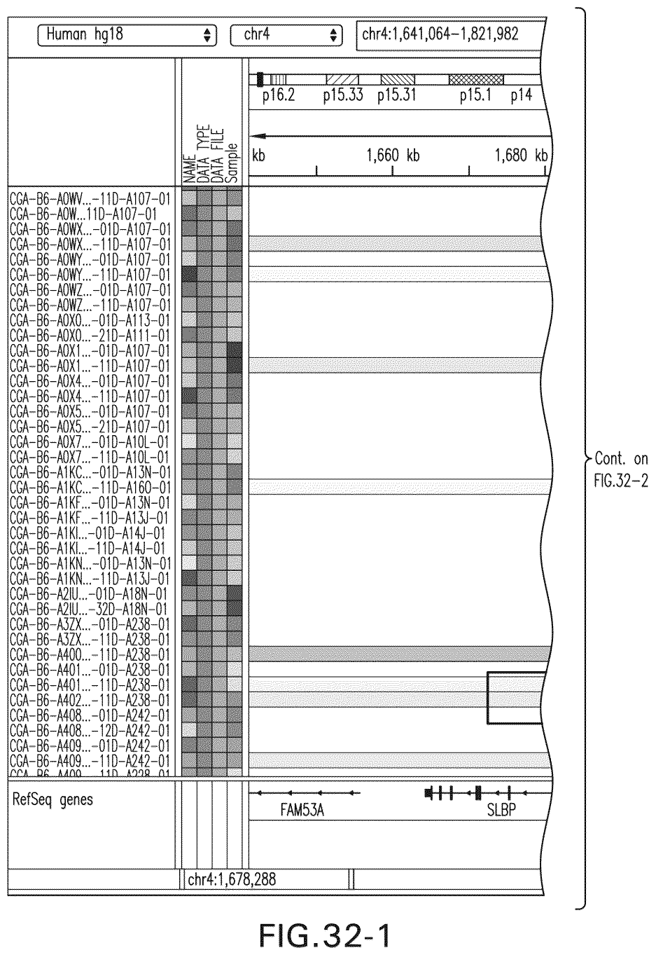

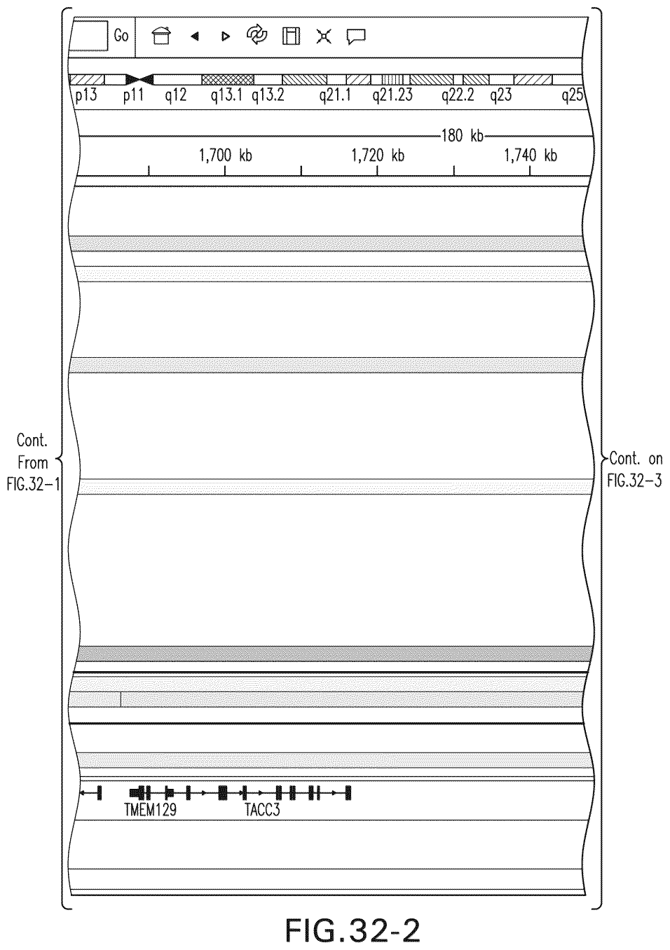

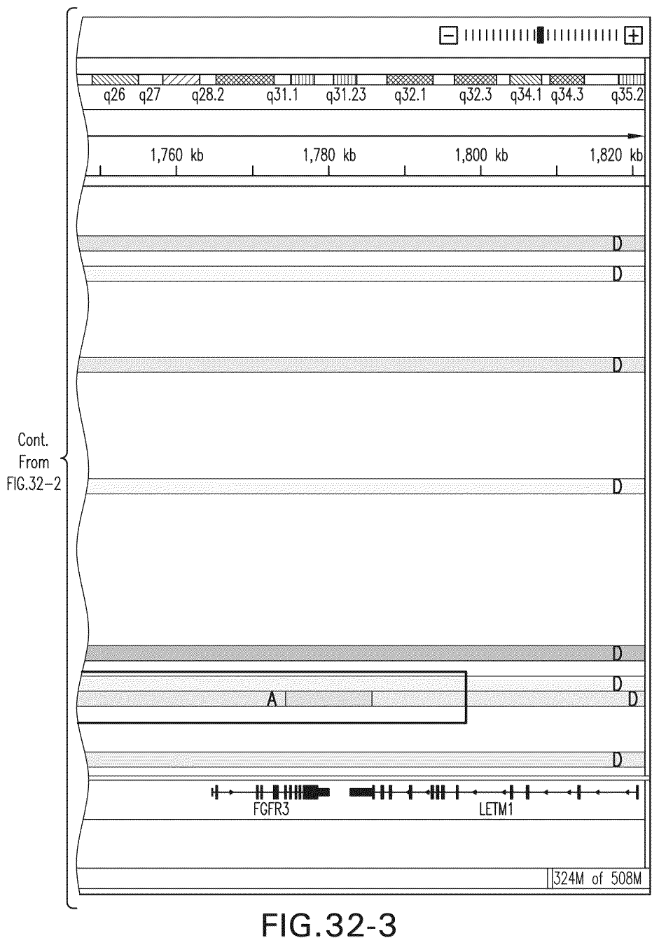

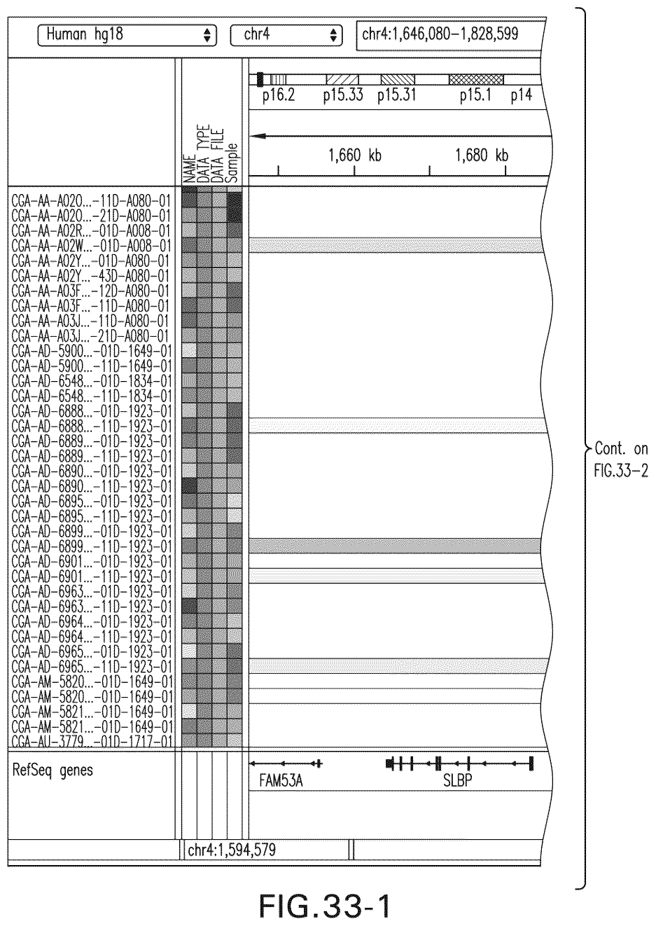

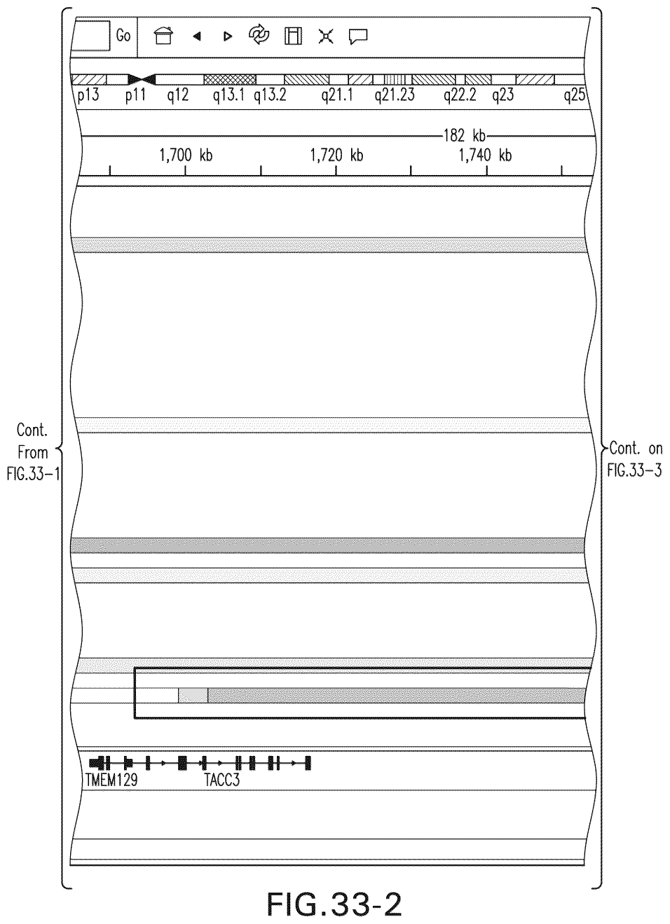

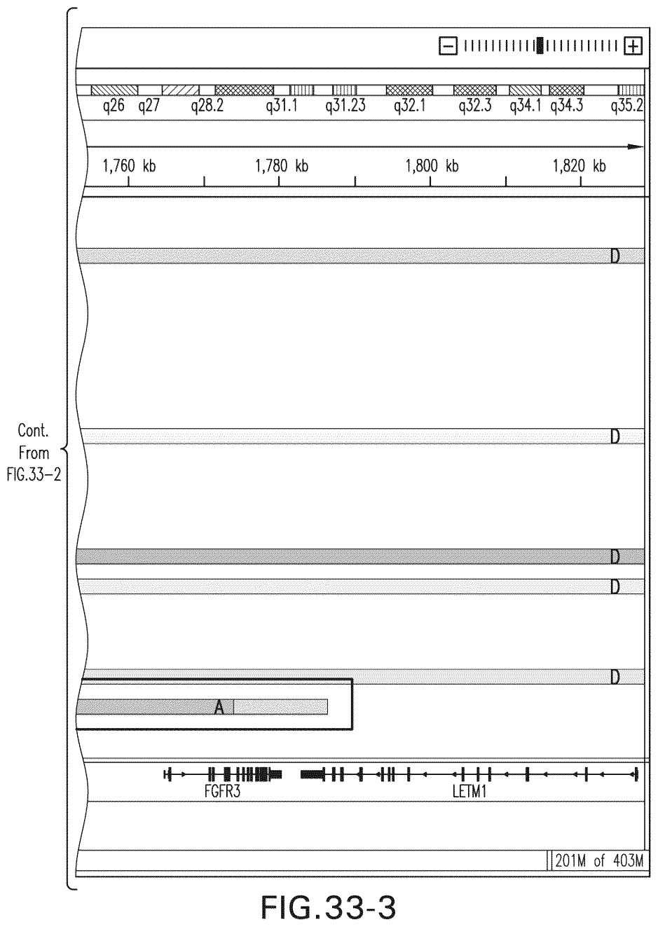

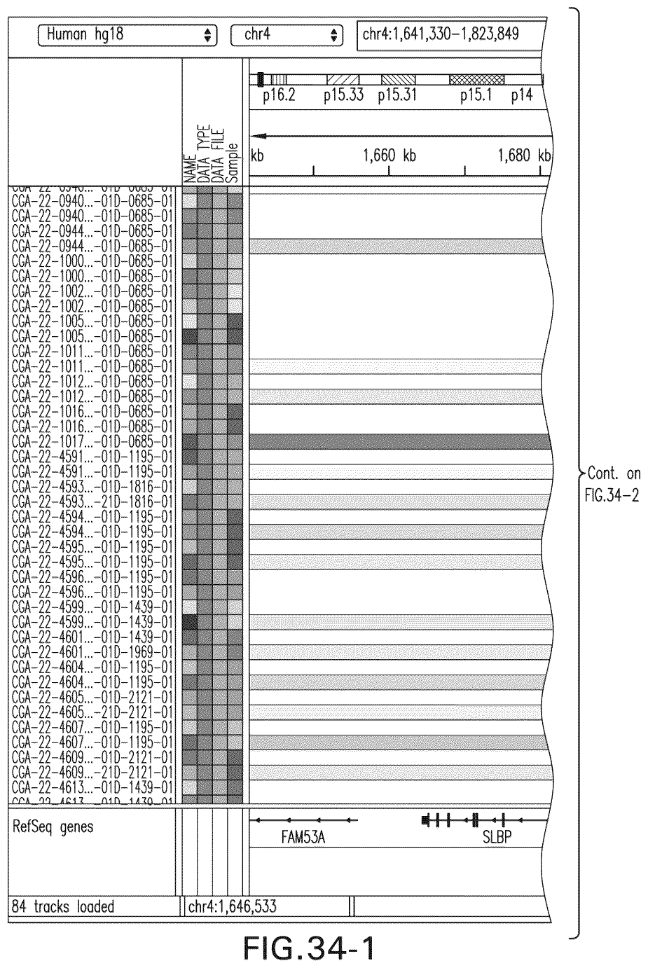

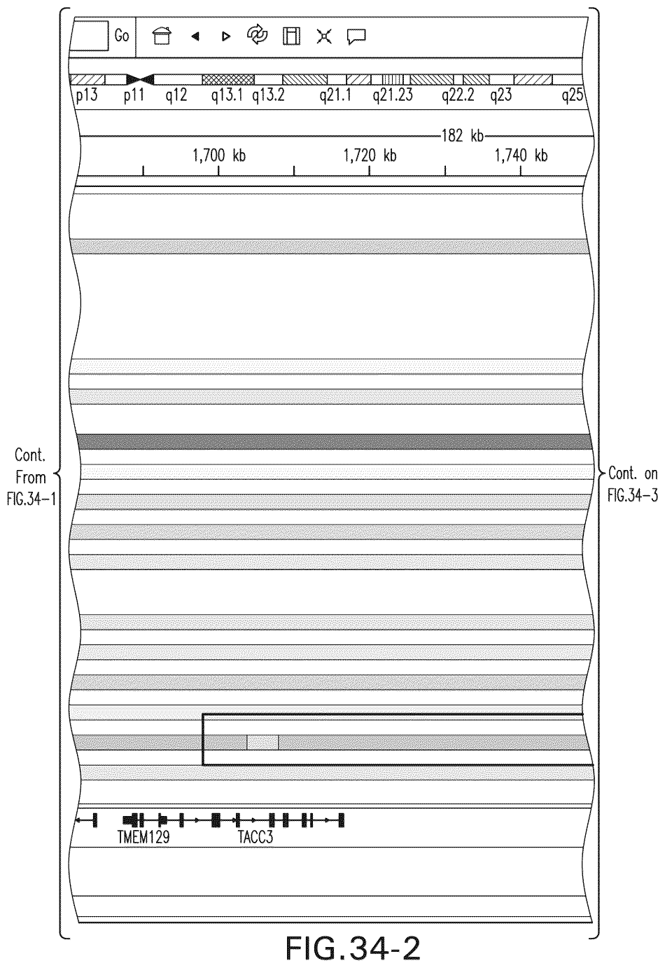

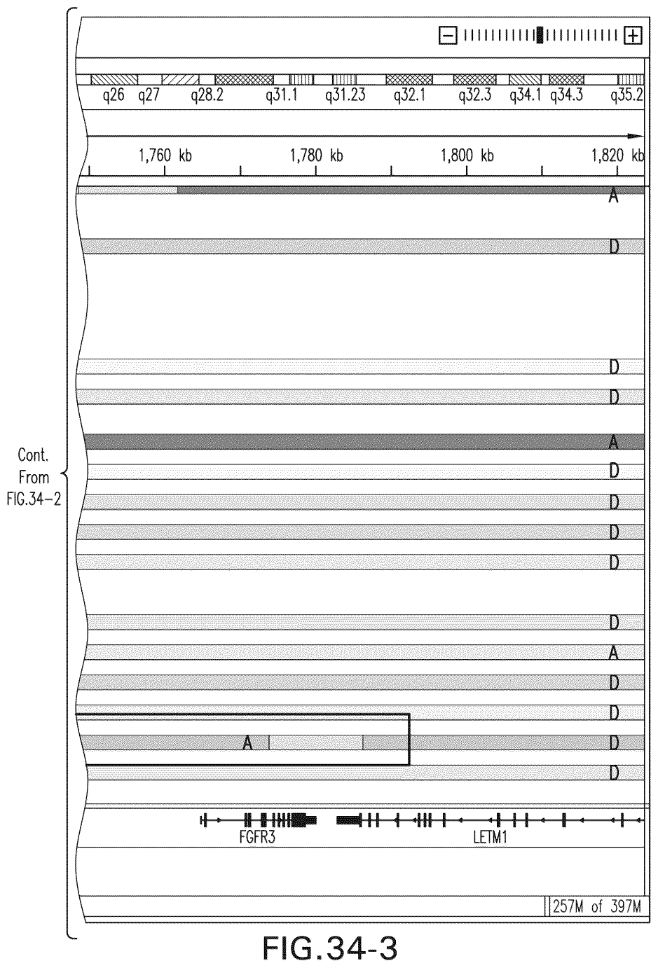

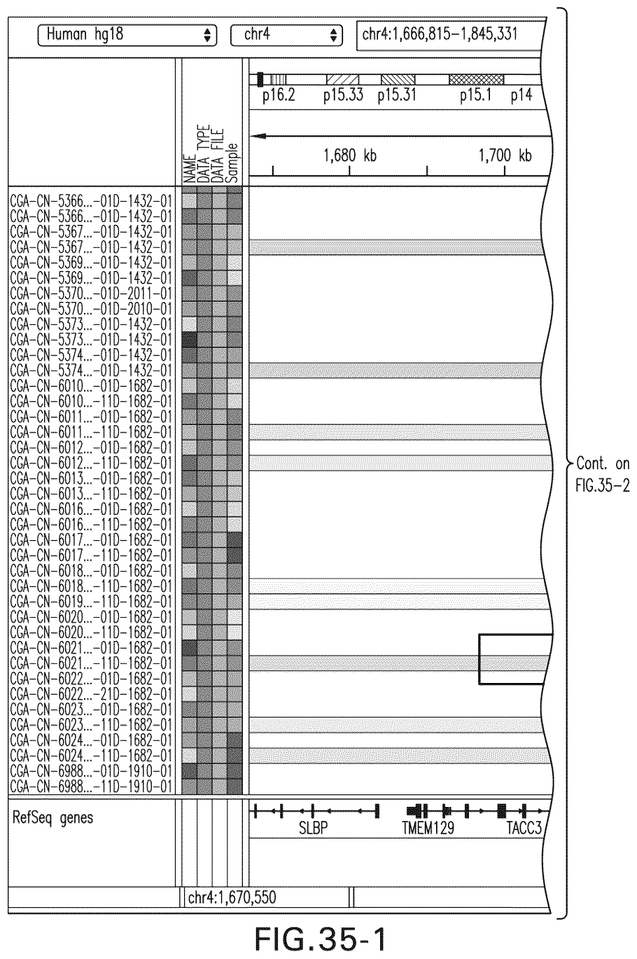

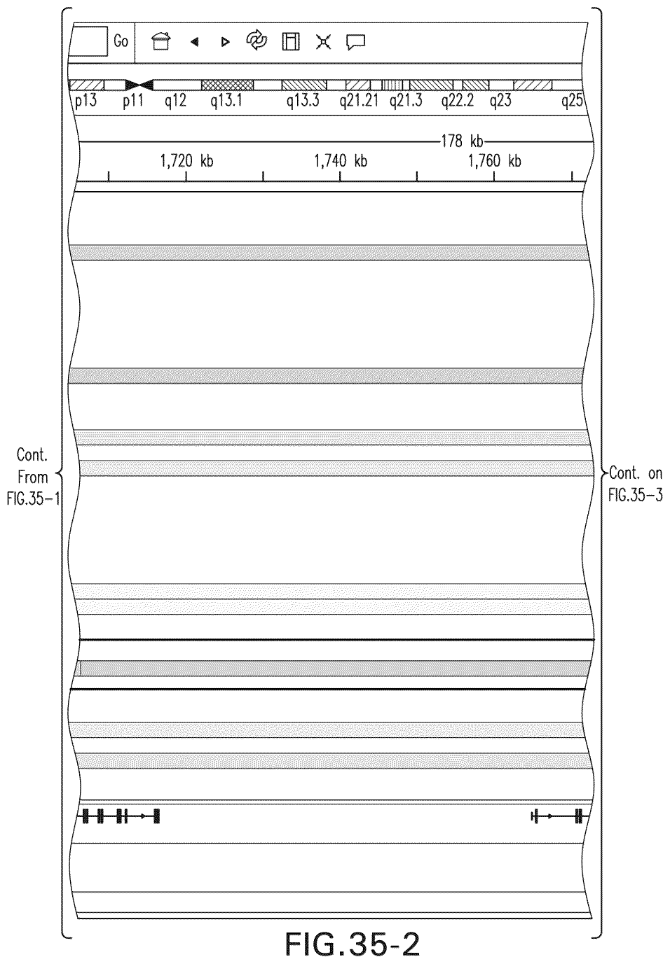

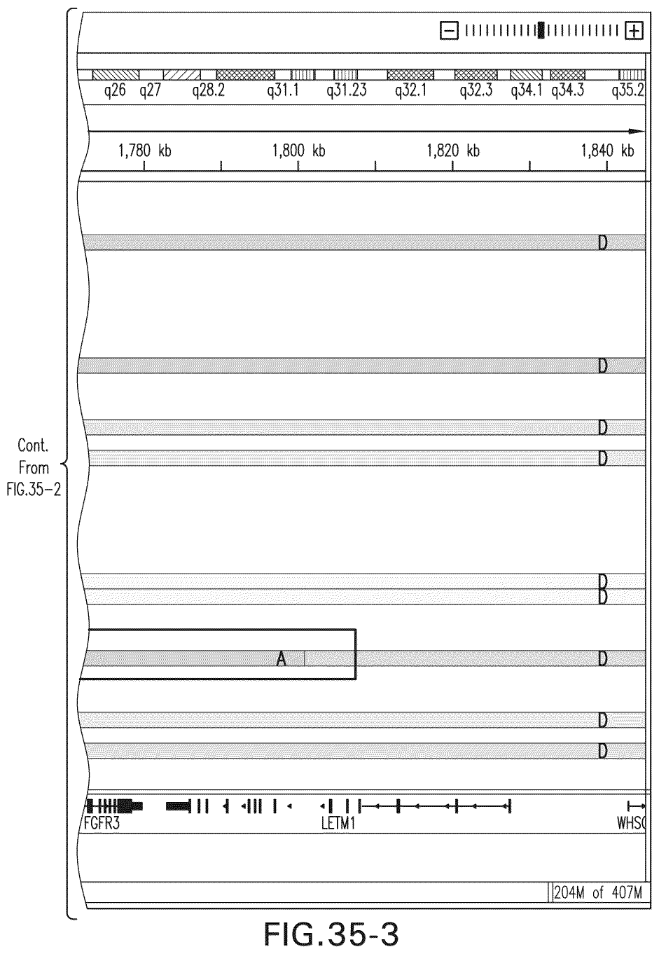

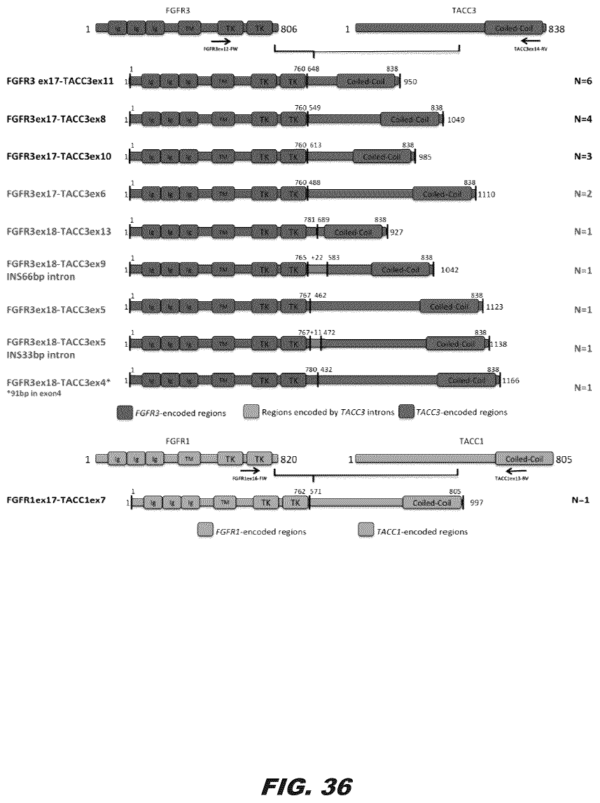

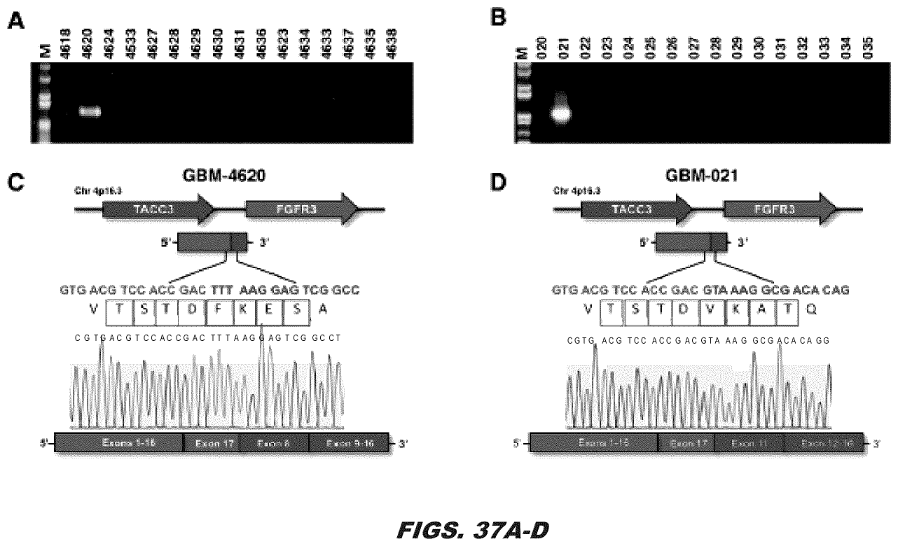

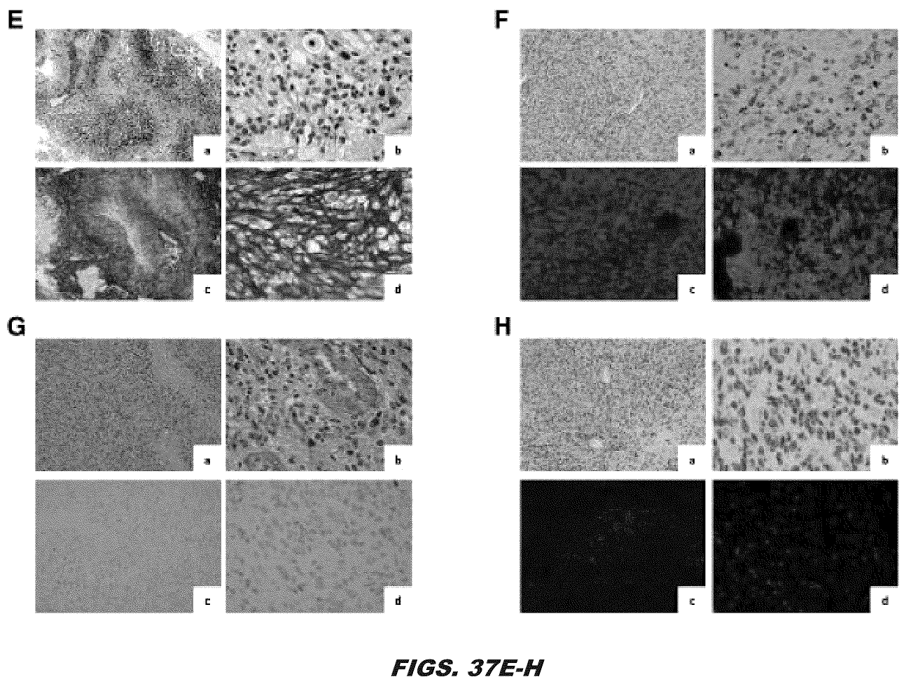

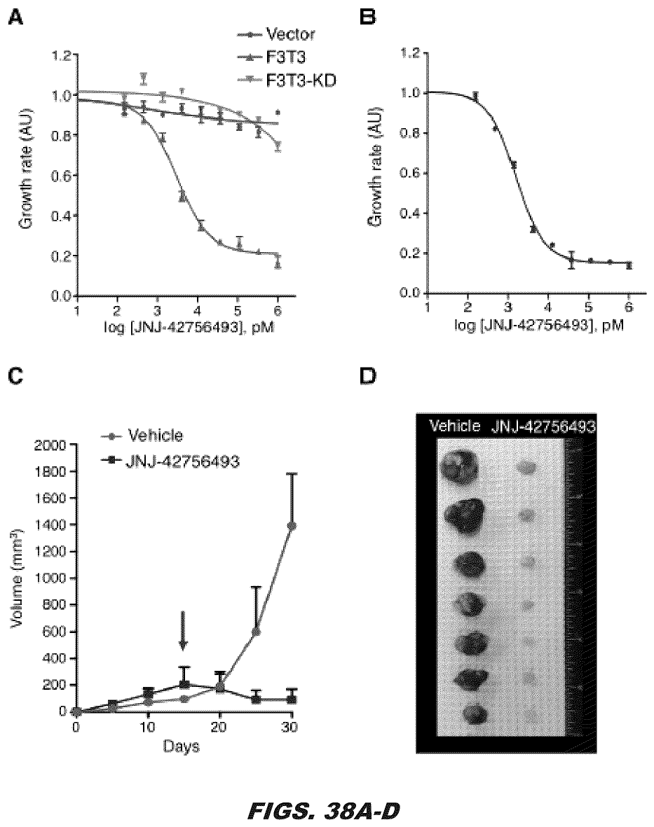

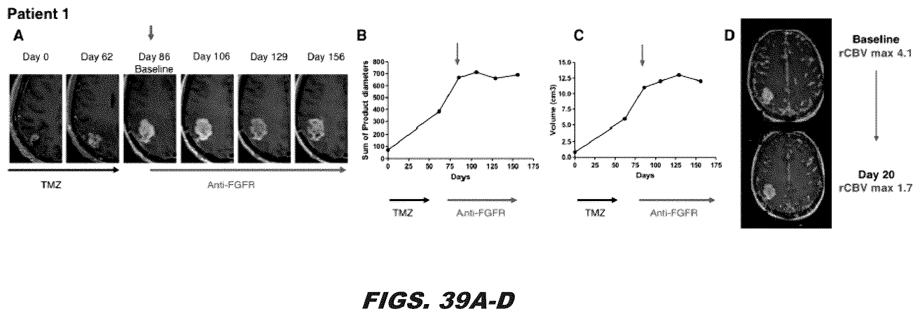

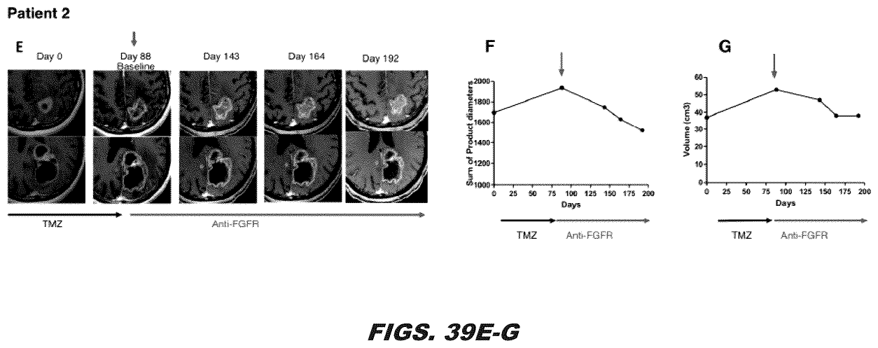

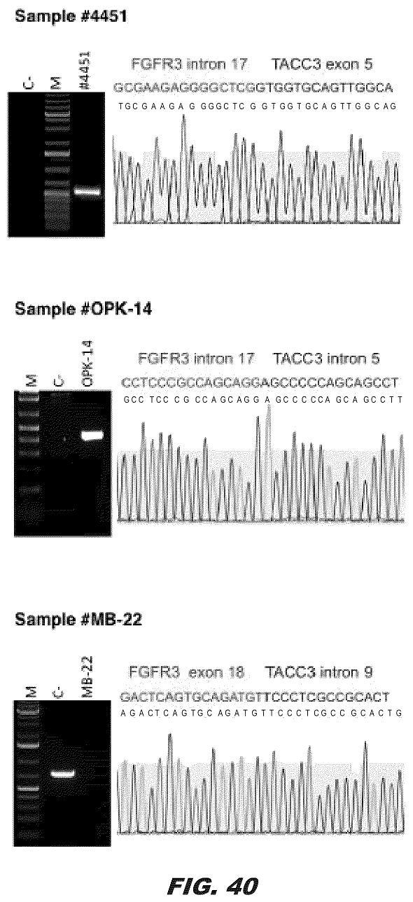

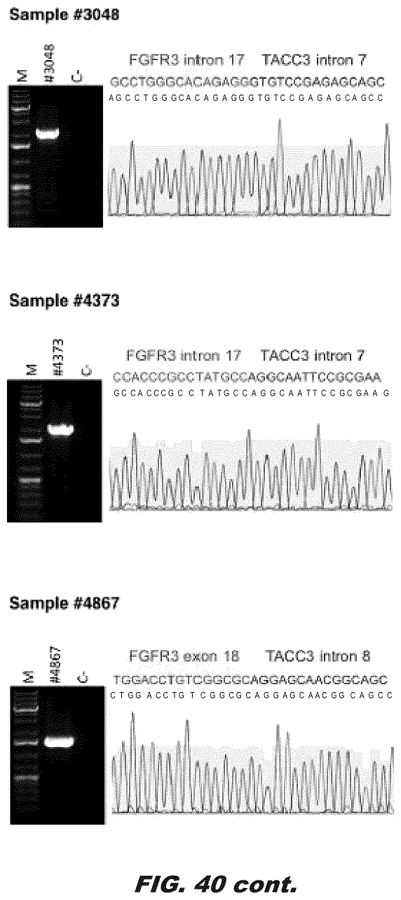

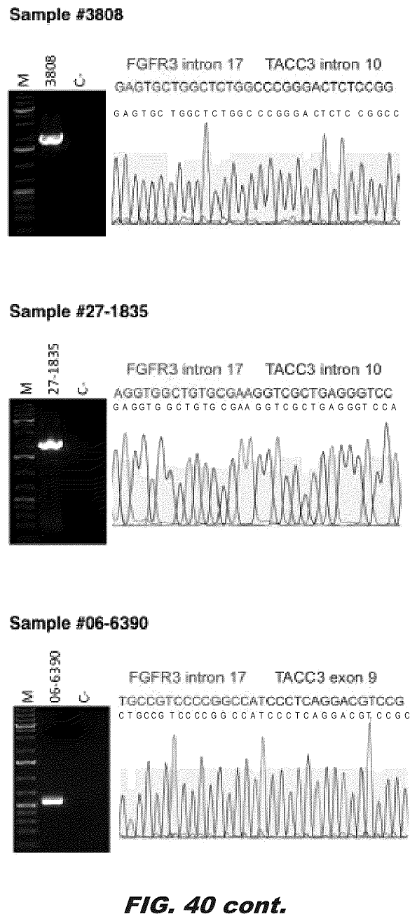

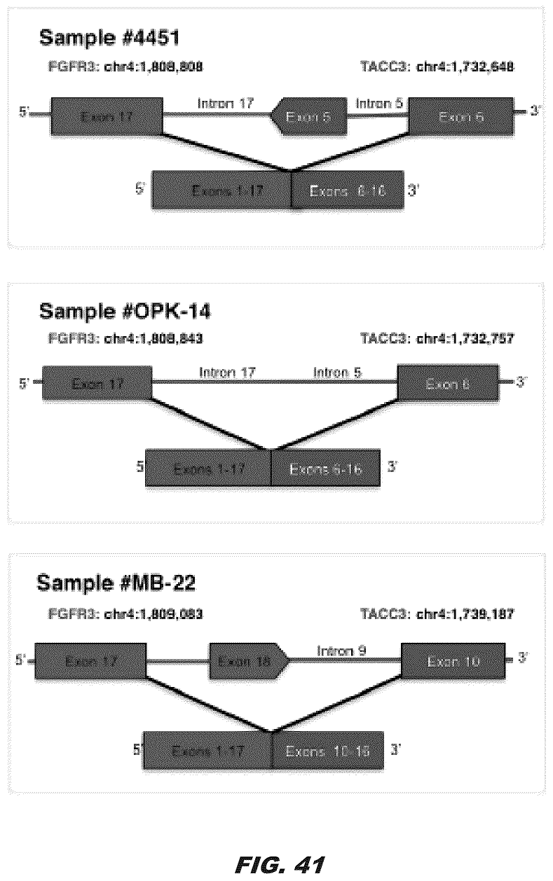

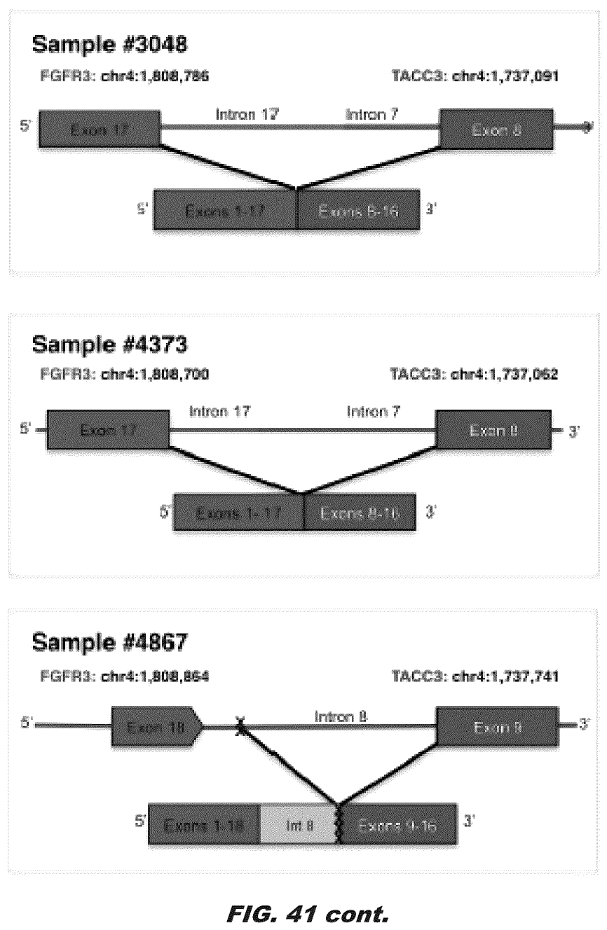

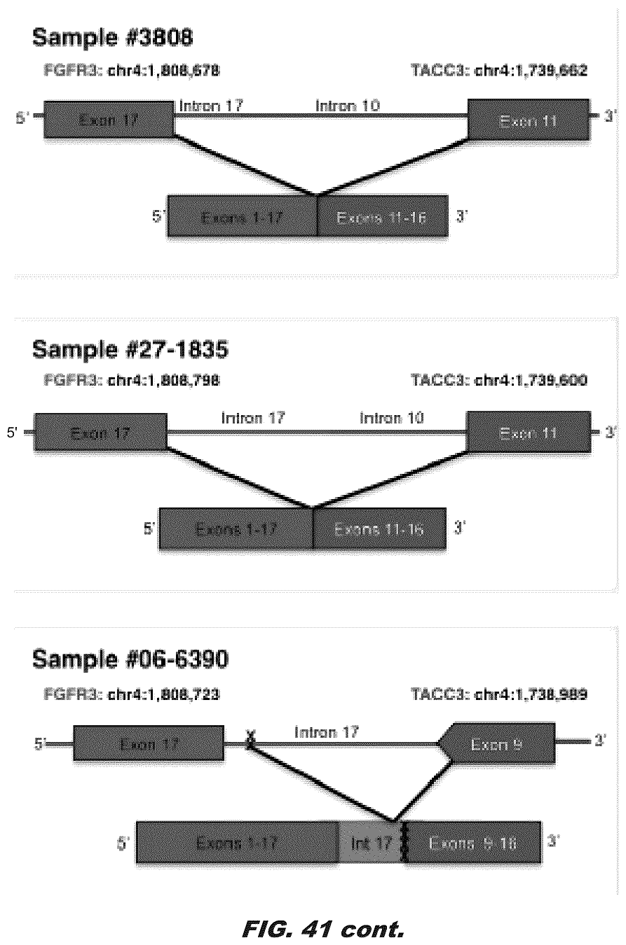

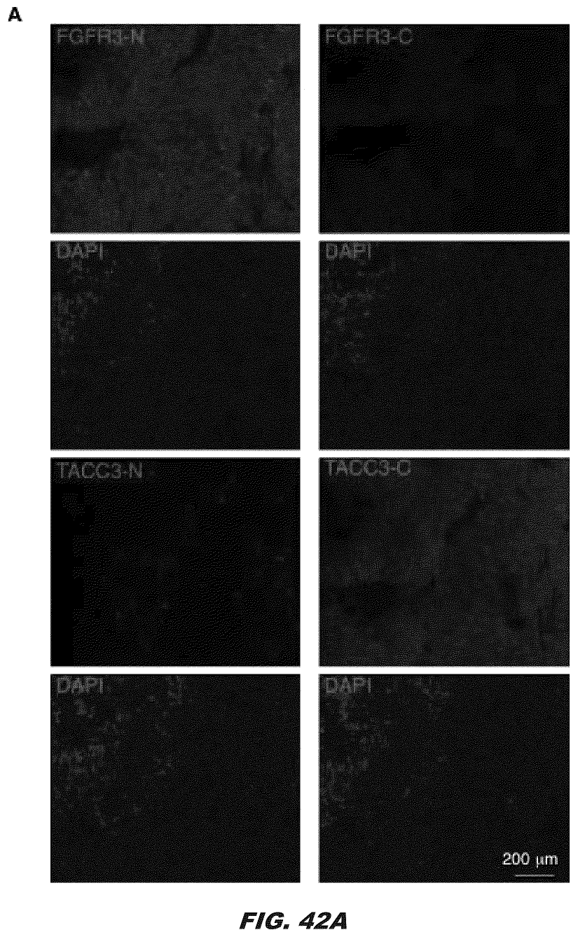

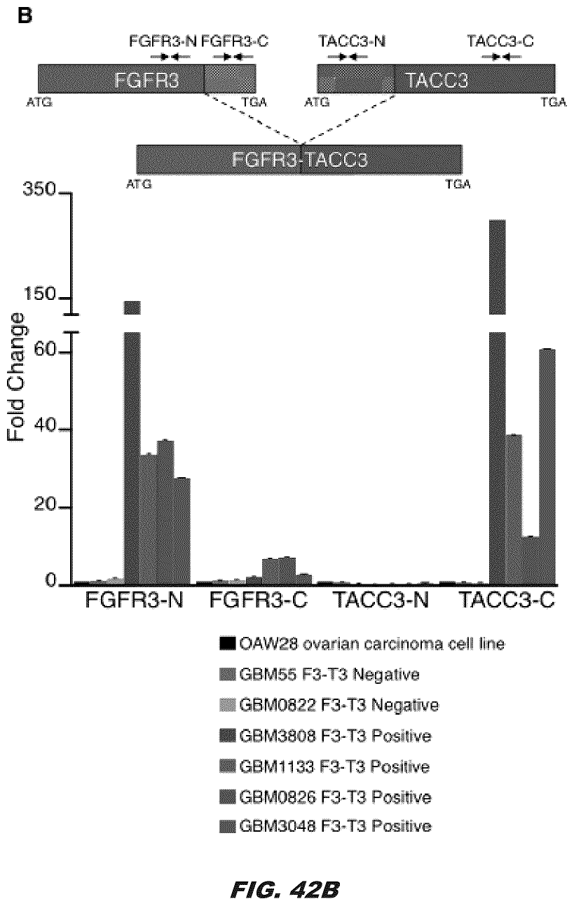

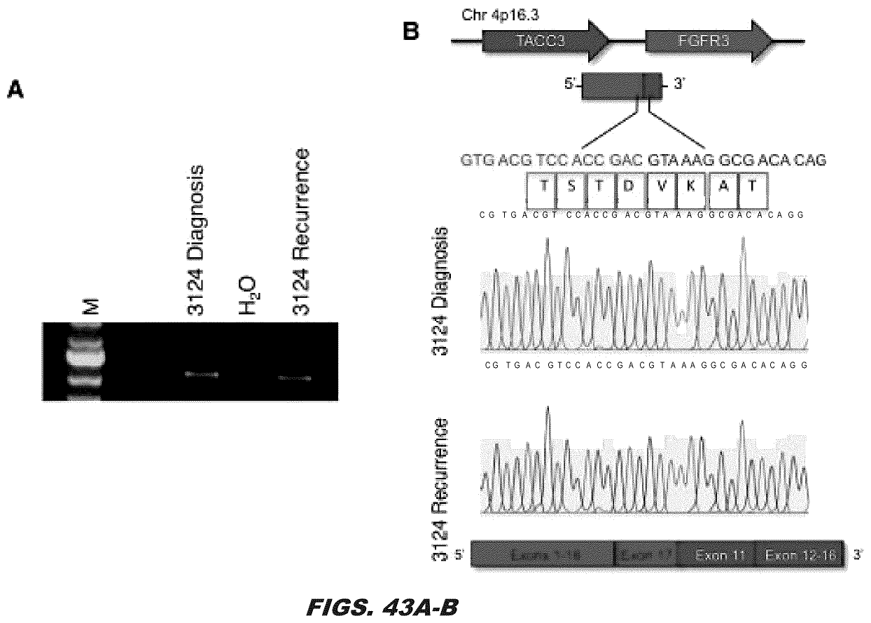

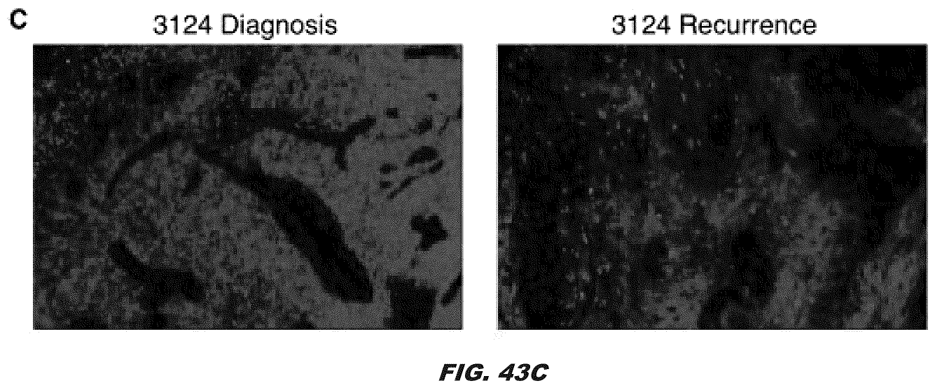

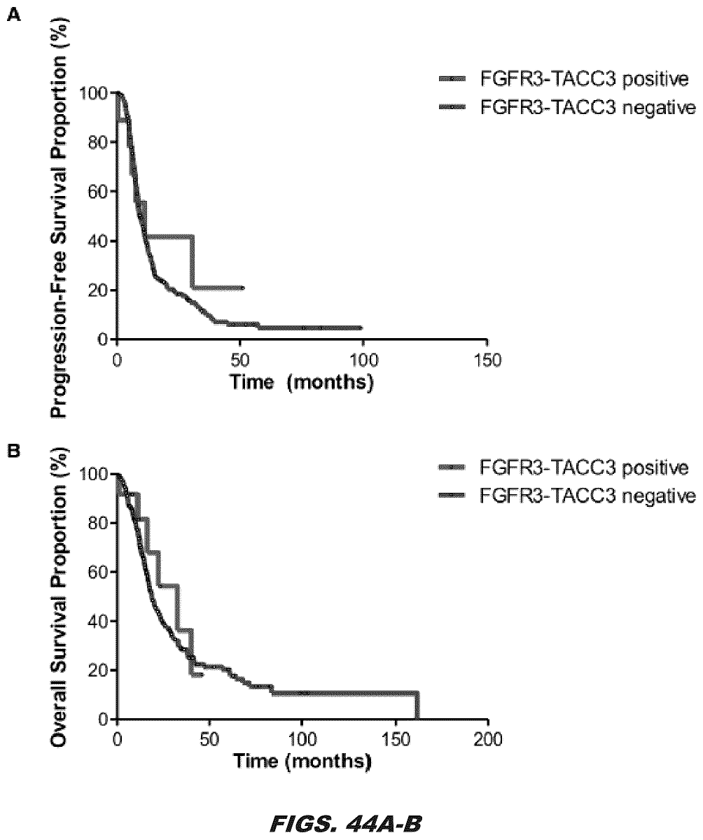

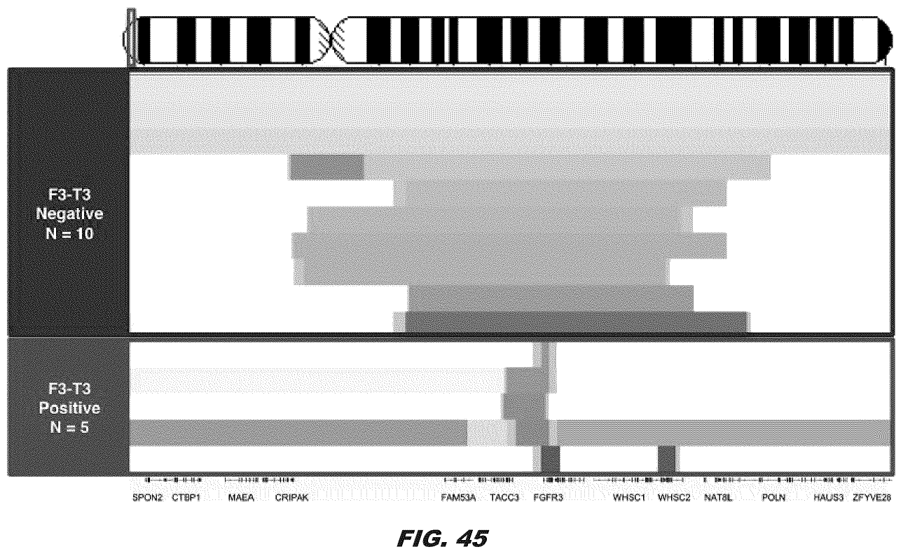

The inventors previously reported in Example 1 that 3.1% of human glioblastoma harbor FGFR3-TACC3 and FGFR-TACC1 gene fusions. Tumors harboring FGFR3-TACC3 gene fusions are identified by the presence of highly specific focal micro-amplification events of the rearranged portions of the FGFR3 and TACC3 genes (See FIG. 2E). Therefore, these micro-amplification events can be used as distinctive marks for the presence of FGFR3-TACC3 gene fusions. It was asked whether other types of human tumors also harbor FGFR3-TACC3 gene fusions from the analysis of Copy Number Variations (CNVs) of SNP arrays generated from the Atlas-TCGA project. This analysis was performed using segmented CNVs data visualized using the Integrated Genomic Viewers software. The analysis revealed that the following tumors, shown in the FIGS. 31-35, display focal micro-amplification events of FGFR3 and TACC3 that indicate the presence of FGFR3-TACC3 gene fusions (in FIGS. 31-35, red indicates amplification (A), blue indicates deletion (D); FIG. 31: Bladder Urothelial Carcinoma; FIG. 32: Breast Carcinoma; FIG. 33: Colorectal Carcinoma; FIG. 34: Lung Squamous Cell Carcinoma; FIG. 35: Head and Neck Squamous Cell Carcinoma).

Taken together, these data indicate that the same FGFR3-TACC3 gene fusions reported for the first time in Glioblastoma also occur in several other types of human tumors. Therefore, as for Glioblastoma and other epithelial cancers (such as the human tumors discussed herein), the identification of FGFR-TACC gene fusions also provides a new diagnostic and therapeutic target for treatment with drugs that inhibit FGFR-TACC gene fusions.

### Example 4—Detection, Characterization and Inhibition of FGFR-TACC Fusions in IDH Wild Type Glioma

1.-23. (canceled)

24. A kit for amplifying a cDNA encoding a FGFR1-TACC1 fusion protein or a complement thereof, or a FGFR3-TACC3 fusion protein or a complement thereof, the kit comprising:
a synthetic polynucleotide probe comprising a polynucleotide sequence complementary to a portion of the cDNA encoding the FGFR1-TACC1 fusion protein or the complement thereof, or a portion of the cDNA encoding the FGFR3-TACC3 fusion protein or the complement thereof,
wherein the synthetic polynucleotide probe further comprises a chemical modification,
wherein the chemical modification comprises a radioactive molecule, a fluorescent molecule, a chemiluminescent molecule, an enzyme, or an affinity molecule, and
wherein the kit is configured to allow for amplification of the cDNA encoding the FGFR1-TACC1 fusion protein or the complement thereof or the FGFR3-TACC3 fusion protein or the complement thereof.

25. The kit of claim 24, wherein the synthetic polynucleotide probe comprises the polynucleotide sequence complementary to the portion of the cDNA encoding the FGFR3-TACC3 fusion protein or the complement thereof.

26. The kit of claim 25, wherein the cDNA encoding the FGFR3-TACC3 fusion protein comprises a tyrosine kinase domain of the FGFR3 portion fused to a TACC domain of the TACC3 portion.

27. The kit of claim 25, wherein the synthetic polynucleotide probe comprises a polynucleotide sequence complementary to a portion of the cDNA encoding a breakpoint between a FGFR3 portion and a TACC3 portion of a FGFR3-TACC3 fusion protein or the complement thereof.

28. The kit of claim 27, wherein the breakpoint between the FGFR3 portion and the TACC3 portion of the FGFR3-TACC3 fusion protein comprises SEQ ID NO: 85, 86, 87, or 89.

29. The kit of claim 27, wherein the synthetic polynucleotide probe sequence comprises SEQ ID NO: 80, 81, 82, or 84, or a complement thereof.

30. The kit of claim 24, wherein the synthetic polynucleotide probe comprises the polynucleotide sequence complementary to the portion of the cDNA encoding the FGFR1-TACC1 fusion protein or the complement thereof.

31. The kit of claim 30, wherein the synthetic probe comprises a polynucleotide sequence complementary to a portion of the cDNA encoding a breakpoint between a FGFR1 portion and a TACC1 portion of the FGFR1-TACC1 fusion protein or the complement thereof.

32. The kit of claim 31, wherein the breakpoint between the FGFR1 portion and the TACC1 portion of the FGFR1-TACC1 fusion protein comprises SEQ ID NO: 88.

33. The kit of claim 31, wherein the synthetic polynucleotide probe sequence comprises SEQ ID NO: 83, or a complement thereof.

34. The kit of claim 24, wherein the synthetic probe is between 10 and 30 nucleotides long.

35. The kit of claim 24, wherein the chemical modification comprises a fluorescent molecule.

36. The kit of claim 35, wherein the fluorescent molecule is capable of being detected during an amplification reaction.

37. The kit of claim 36, wherein the amplification reaction is RT-PCR.
